(19) United States
(12) Patent Application Publication
(10) Pub. No.: US 2023/0340073 A1
(43) Pub. Date: Oct. 26, 2023

(54) RECOMBINANT FACTOR VIII VARIANTS

(71) Applicant: Codexis, Inc., Redwood City, CA (US)

(72) Inventors: Jeremiah Dallas Heredia, Sunnyvale, CA (US); Nikki D. Kruse, San Carlos, CA (US); Aksiniya Lyubenova Petkova, Sunnyvale, CA (US); Marcus Rohovie, Redwood City, CA (US); Adam P. Silverman, San Carlos, CA (US)

(21) Appl. No.: 18/150,761

(22) Filed: Jan. 5, 2023

Related U.S. Application Data
(60) Provisional application No. 63/297,690, filed on Jan. 7, 2022.
(60) Provisional application No. 63/478,454, filed on Jan. 4, 2023.

(51) Int. Cl. C07K 14/755; C12N 15/63; A61P 7/04

(52) U.S. Cl. CPC C07K 14/755; C12N 15/63; A61P 7/04

(57) ABSTRACT

The present disclosure provides recombinant factor VIII polypeptides, polynucleotides encoding the recombinant factor VIII polypeptides, and compositions thereof, wherein the recombinant factor VIII polypeptides have been engineered to provide improved expression, activity/potency, clotting activity/potency, serum/plasma stability, and/or reduced immunogenicity. The present disclosure also relates to the use of the compositions comprising the recombinant factor VIII polypeptides or polynucleotides encoding the recombinant factor VIII polypeptides for therapeutic purposes.

### CROSS-REFERENCE TO RELATED APPLICATIONS

This application claims the benefit under 35 U.S.C. § 119(e) of U.S. Provisional Application No. 63/297,690, filed Jan. 7, 2022, and U.S. Provisional Application No. 63/478,454, filed Jan. 4, 2023, the contents of which are incorporated by reference herein in their entireties.

### REFERENCE TO SEQUENCE LISTING, TABLE OR COMPUTER PROGRAM

The Sequence Listing submitted concurrently herewith with a file name of “CX7-224US2_ST26.xml,” a creation date of Jan. 5, 2023, and a size of 11.4 megabytes, is part of the specification and is incorporated by reference herein.

### TECHNICAL FIELD

The present disclosure provides recombinant factor VIII polypeptide variants and compositions thereof. The recombinant factor VIII polypeptides have been engineered to have improved properties, such as improved expression, thermostability, serum stability, plasma stability, stability under basic conditions, reduced immunogenicity, improved cofactor potency, and improved blood clotting. The present disclosure also relates to the use of the compositions comprising the recombinant factor VIII polypeptides for therapeutic purposes.

### BACKGROUND

Human factor VIII (“FVIII”), also known as clotting factor VIII or anti-hemophilic factor, is a protein cofactor that functions as a necessary part of the blood coagulation pathway which in response to injury initiates a chain of enzymatic and chemical reactions that results in a blood clot. Factor VIII directly supports Factor IX as a cofactor, improving its enzymatic activity by 10⁵-fold (see e.g., Mazurkiewicz-Pisarek et al., Acta Biochimica Polonica, 2016, 63.1:11-16). Hemophilia A, also referred to as haemophilia A, is an X-linked genetic deficiency in factor VIII that results in increased bleeding and lack of blood clots. There are three generally recognized forms of hemophilia A. Patients who have mild hemophilia A have excessive bleeding after trauma or surgery but are otherwise healthy. Patients who have moderate hemophilia A also suffer from joint and muscle bleeding after mild trauma. Patients who have severe hemophilia A suffer from spontaneous joint and muscle bleeding even without trauma. All patients with hemophilia A require treatment, but patients with the severe form and some patients with the moderate form require regular and frequent infusions of factor VIII to prevent excessive blood loss and death (see, e.g., Peters et al., Nature Reviews Drug Discovery, 2018, 17(7):493-508). This disease is estimated to affect 1 in 5,000 males, but also occurs in females. Treatment is generally supportive and there is no cure, thus there remains a need for a safe and effective treatment.

### SUMMARY

The present disclosure provides recombinant factor VIII polypeptide variants and compositions thereof. The recombinant factor VIII polypeptide variants have been engineered to have improved properties, including increased activity, thermostability, serum stability, plasma stability, reduced immunogenicity, and/or improved blood clotting. The disclosure also relates to the use of the recombinant factor VIII polypeptides for therapeutic purposes.

In one aspect, the present disclosure provides a recombinant factor VIII polypeptide, or abiologically active fragment thereof, comprising an amino acid sequence having at least 70%, 75%, 80%, 81%, 82%, 83%, 84%, 85%, 86%, 87%, 88%, 89%, 90%, 91%, 92%, 93%, 94%, 95%, 96%, 97%, 98%, or 99% or more sequence identity to a reference sequence corresponding to residues 20-1457 of SEQ ID NO: 2, 252, 504, 706, 840, 1200, 1514, or 2072, or to a reference sequence corresponding to SEQ ID NO: 2, 252, 504, 706, 840, 1200, 1514, or 2072, wherein the amino acid sequence comprises one or more substitutions relative to the reference sequence corresponding to residues 20-1457 of SEQ ID NO: 2, 252, 504, 706, 840, 1200, 1514, or 2072, or relative to the reference sequence corresponding to SEQ ID NO: 2, 252, 504, 706, 840, 1200, 1514, or 2072.

In some embodiments, the recombinant factor VIII polypeptide comprises an amino acid sequence having at least 70%, 75%, 80%, 81%, 82%, 83%, 84%, 85%, 86%, 87%, 88%, 89%, 90%, 91%, 92%, 93%, 94%, 95%, 96%, 97%, 98%, or 99% or more sequence identity to the reference sequence corresponding to residues 20-1457 of SEQ ID NO: 2 or to the reference sequence corresponding to SEQ ID NO: 2, wherein the amino acid sequence comprises one or more substitutions relative to the reference sequence corresponding to residues 20-1457 of SEQ ID NO: 2, or relative to the reference sequence corresponding to SEQ ID NO: 2.

In some embodiments, the recombinant factor VIII polypeptide comprises an amino acid sequence having at least 70%, 75%, 80%, 81%, 82%, 83%, 84%, 85%, 86%, 87%, 88%, 89%, 90%, 91%, 92%, 93%, 94%, 95%, 96%, 97%, 98%, or 99% or more sequence identity to the reference sequence corresponding to residues 20-1457 of SEQ ID NO: 252, 504, 706, 840, 1200, 1514, or 2072, or to the reference sequence corresponding to SEQ ID NO: 252, 504, 706, 840, 1200, 1514, or 2072, wherein the amino acid sequence comprises one or more substitutions relative to the reference sequence corresponding to residues 20-1457 of SEQ ID NO: 2, or relative to the reference sequence corresponding to SEQ ID NO: 2.

In some embodiments, the amino acid sequence of the recombinant factor VIII polypeptide comprises at least a substitution at amino acid position 13, 14, 15, 16, 17, 18, 19, 20, 21, 24, 37, 56, 57, 58, 59, 61, 63, 64, 65, 85, 96, 104, 110, 119, 127, 132, 158, 160, 164, 167, 168, 171, 175, 178, 186, 187, 205, 210, 211, 212, 215, 225, 246, 257, 260, 265, 281, 292, 294, 301, 303, 304, 306, 308, 310, 311, 312, 318, 320, 321, 325, 328, 332, 334, 335, 344, 346, 347, 349, 351, 353, 354, 355, 356, 358, 359, 362, 370, 387, 388, 390, 392, 393, 397, 407, 428, 429, 433, 435, 436, 481, 487, 488, 489, 491, 498, 499, 503, 514, 527, 533, 534, 535, 536, 538, 543, 550, 551, 555, 558, 569, 580, 581, 584, 610, 611, 612, 613, 614, 615, 616, 617, 618, 619, 620, 621, 622, 643, 646, 650, 665, 667, 676, 680, 681, 682, 683, 684, 686, 693, 696, 698, 700, 706, 707, 708, 712, 716, 724, 726, 729, 731, 732, 734, 735, 736, 737, 738, 739, 740, 744, 746, 747, 748, 749, 750, 751, 752, 753, 754, 755, 756, 757, 759, 770, 773, 775, 785, 786, 787, 790, 791, 792, 793, 794, 795, 797, 798, 799, 800, 801, 802, 805, 806, 807, 808, 809, 810, 811, 812, 815, 816, 817, 818, 819, 821, 822, 824, 825, 826, 828, 830, 831, 836, 837, 838, 839, 840, 841, 844, 866, 873, 879, 892, 896, 897, 903, 907, 914, 916, 917, 918, 919, 921, 922, 923, 924, 926, 929, 938, 939, 945, 948, 951, 968, 999, 1037, 1040, 1041, 1042, 1043, 1049, 1050, 1051, 1053, 1054, 1076, 1078, 1080, 1084, 1088, 1089, 1092, 1093, 1094, 1095, 1098, 1099, 1100, 1101, 1104, 1108, 1109, 1113, 1114, 1116, 1127, 1129, 1138, 1145, 1148, 1151, 1156, 1158, 1160, 1186, 1192, 1193, 1194, 1205, 1210, 1218, 1220, 1221, 1222, 1224, 1229, 1245, 1248, 1250, 1259, 1264, 1269, 1270, 1274, 1281, 1283, 1292, 1297, 1339, 1352, 1373, 1377, 1378, 1380, 1389, 1414, 1422, 1427, 1428, 1439, 1440, 1442, or 1452, or combinations thereof, wherein the amino acid positions are relative to the reference sequence corresponding to SEQ ID NO: 2.

In some embodiments, the amino acid sequence of the recombinant factor VIII polypeptide comprises at least a substitution or amino acid residue 13G/Y, 14G/M/V, 15G/S, 16A/D/G/P/V, 17S/V, 18L/R, 19G, 20L, 21M, 24V, 37H, 56E/P, 57A/L/S/T/V, 58K, 59D/G/L/M/Q/R/S/V/W, 61Q/S, 63C/E/F/G/H/I/K/L/M/N/Q/R/S/T/W/Y, 64D/G, 65A, 85Q, 96I/L/R/V/W, 104S, 110H, 119G/V, 127V, 132P/R, 158G, 160E/T, 164E, 167G/H/R, 168G/R, 171A/D/E/F/H/K/N/M/P/Q/R/S/T/V/W/Y, 175A/G/H, 178C/M/Q/V/W, 186A, 187C/G, 205R, 210P, 211G/K/P/Q/R, 212A/F/G/K/L/M/N/P/R/S/V/Y, 215E/P/S, 225R, 246D, 257I, 260E/K/T, 265R/S/W, 281A/S, 292D, 294G, 301K/M, 303G/T, 304A/E/G, 306A/D/G/P/S/T, 308P, 310G/V, 311E/G, 312L, 318C/G/K/M/Q/R/T/V, 320G/I/L/P/Q/R/S/T/V, 321Y, 325Y, 328G/Q/S, 332A/F/G/H/N/P/Q/R/T/V/W/Y, 334G, 335E/P, 344A/E/L, 346R/V, 347N, 349G, 351S, 353S, 354R, 355C, 356L/N, 358K/S, 359E/G, 362E/G/N/P/T/V/W, 370A, 387A/G/I/L/Q/R/S/T/V/W, 388A/P, 390A/E/G/L/M/P/S/T/V, 392A, 393A/F/G/H/M/Q/R/W/Y, 397G/N, 407V, 428F, 429L/S, 433A/G, 435Q, 436D/G, 481V, 487N, 488G, 489E/F/Q/R, 491E, 498A, 499C/L, 503S, 514L, 527A, 533R/V/Y, 534F/I/L/M, 535C/G/N, 536A/G/K/L/M/P/R/W, 538V, 543G/L/P/W, 550G, 551W, 555E/G/R/T, 558A/E/G/L/P/Q/R/S/T/V/W, 569T, 580S/Y, 581K/V, 584A, 610H/K/L/R/S/T, 611D/E, 612G/S/T, 613S, 614A/F/H/I/N/T, 615D/N, 616D/E/G/Q/W, 617D/E/G/N, 618C/E/G/P/Y, 619E, 620N/P, 621K/M/R/T/W, 622D, 643N, 646I, 650M, 665G, 667I, 676Q, 680L/R, 681A/C/E/F/G/L/N/R/Q/S/T/W, 682R, 683W, 684G/K, 686W, 693T, 696E, 698D/S/W/Y, 700A/E/Q/T/V, 706A/G/M/Y, 707H, 708A/K/T/V, 712G/L/M/Q/S/T/Y, 716K/L/W, 724I, 726A/L, 729T, 731I/L/R, 732C/D/E/G/L/R/W/K/M/Q/S/T/V/Y, 734I/R/S, 735D/S/V, 736F/G, 737F/G/K/L/M/N/S/V, 738A/D/E/F/H/K/N/P/R/S/T, 739G/P/R, 740G, 744A/R, 746P, 747D, 748F/G/K/N/S/V, 749A/C/D/F/G/I/K/L/M/P/Q/R/S/T/V/Y, 750A/D/G/K/M/N/P/Q/R/S/T/V/W, 751A/D/E/Y, 752D/E/F/G/L/M/Q/R/S/V, 753D, 754W, 755G/L/P/Q/S/Y, 756G/P/R/T, 757A/G/K/T, 759S, 770W, 773H, 775E/G/K/N/P/S/V, 785V, 786C/D/L, 787W, 790A/G/I/M/P/W, 791M/T, 792H/W/Y, 793G, 794R, 795C/N/S/T, 797C/G/P/Q/T/W/Y, 798C/R/T, 799A/E/G/M/P/Q/R/S/V, 800C/G/I/L/M/N/P/Q/R/T/V/W/Y, 801A/G/I/R, 802S/V, 805C/G/H/K/L/M/P/Q/R/S/V/W, 806E/G/R/S, 807C/L/P/R, 808K/S, 809A/C/G/I/K/L/N/Q/R/S/T/V/W, 810V, 811F, 812G/I/V, 815I, 816V/W, 817A/D, 818A/E/G/P/S/V/W, 819V, 821Q, 822S/T, 824W, 825L, 826G, 828K, 830M/V, 831W, 836G/R/V/W, 837G, 838G/T, 839A/G/K/P, 840G, 841W, 844H, 866A, 873L/Q/T, 879Q, 892T, 896L/V, 897G/K/R, 903E, 907D, 914V, 916C/E/I/K/L/M/Q/T, 917G, 918M/S, 919G, 921R/W, 922W, 923M, 924I/M/P/Q/W, 926T/V, 929A/Q/V, 938R/V, 939E/S, 945K, 948S, 951V, 968A/P, 999R, 1037H/M/N/P, 1040E, 1041G/H/N, 1042A/E/T, 1043E/R, 1049G/W, 1050E/P/Q/S/T, 1051E/H/K/R/S/W, 1053A/R, 1054C, 1076G/S, 1078S, 1080G/V, 1084L/N, 1088G/S, 1089F/S, 1092G, 1093A/E/G/M/N/P/Q/R/S/T/V/W/Y, 1094S, 1095G, 1098G, 1099G, 1100I/S, 1101G, 1104C/E, 1108M/S/V/W, 1109G, 1113F, 1114A/M, 1116A/G/I/L/R, 1127L/V, 1129P, 1138Q, 1145R, 1148A/C/E/L/M/N/Q/R/S, 1151S, 1156D/G/N/R, 1158N, 1160R, 1186E, 1192D, 1193A/E/G/I/L, 1194P, 1205L, 1210M/T/V/W, 1218A/G/L/M/P, 1220D/E/L/T, 1221G/S/T/V, 1222F, 1224P, 1229D, 1245R, 1248A/C/D/E/F/G/H/I/K/M/P/Q/R/S/T/W/Y, 1250A/P/S/T, 1259T, 1264L/R/W, 1269F, 1270V, 1274N/V, 1281H/K/P/Q/R/S, 1283E/H/L/S/T, 1292Y, 1297A, 1339C, 1352E, 1373S, 1377A/C/D/E/G/H/I/M/P/R/S/V/T/Y, 1378A/C/D/E, 1380A/G/W, 1389W, 1414A/C/D/E/G/H/N/P/T/W, 1422I, 1427Y, 1428G, 1439A/E/H/L/S/T, 1440A/C/E/G/S/T, 1442T, or 1452L, or combinations thereof, wherein the amino acid positions are relative to the reference sequence corresponding to SEQ ID NO: 2.

In some embodiments, the amino acid sequence of the recombinant factor VIII polypeptide comprises at least a substitution at amino acid position 63, 171, 212, 260, 306, 317, 320, 332, 387, 390, 393, 534, 558, 681, 732, 738, 749, 750, 775, 1148, 1248, 1377, or 1439, or combinations thereof, wherein the amino acid positions are relative to the reference sequence corresponding to SEQ ID NO: 2.

In some embodiments, the amino acid sequence of the recombinant factor VIII polypeptide comprises at least a substitution or amino acid residue 63C/E/F/G/H/I/K/L/M/N/Q/R/S/T/W/Y, 171A/D/E/F/H/K/N/M/P/Q/R/S/T/V/W/Y, 212A/F/G/L/K/M/N/P/R/S/V/Y, 260E/K/T, 306A/D/G/P/S/T, 318C/G/K/L/M/Q/R/T/V, 320G/I/L/P/Q/R/S/T/V, 332A/F/G/H/N/P/Q/R/T/V/W/Y, 387A/G/I/L/Q/R/S/T/V/W, 390A/E/G/L/M/P/S/T/V, 393A/F/G/H/M/Q/R/W/Y, 499C/L, 534I/F/L/M, 558A/E/G/L/P/Q/R/S/T/V/W, 681A/C/E/F/G/L/N/R/Q/S/T/W, 700A/E/Q/T/V, 732C/D/E/G/L/R/W/K/M/Q/S/T/V/Y, 738A/D/E/F/H/K/N/P/R/S/T/Y, 749A/C/D/F/G/I/K/L/M/P/Q/R/S/T/V/Y, 750A/D/G/K/M/N/P/Q/R/S/T/V/W, 752D/E/F/G/L/M/Q/R/S/V, 775E/G/K/N/P/S/V, 800C/G/I/L/M/N/P/Q/R/T/V/W/Y, 805C/G/H/K/L/M/P/Q/R/S/V/W, 809A/C/G/I/K/L/N/Q/R/S/T/V/W, 1093A/E/G/M/N/P/Q/R/S/T/V/W/Y, 1148A/C/E/L/M/N/Q/R/S, 1248A/C/D/E/F/G/H/I/K/M/P/Q/R/S/T/W/Y, 1377A/C/D/E/G/H/I/M/P/R/S/V/T/Y, 1414A/C/D/E/G/H/N/P/T/W, or 1439A/E/H/L/S/T, or combinations thereof, wherein the amino acid positions are relative to the reference sequence corresponding to SEQ ID NO: 2.

In some embodiments, the amino acid sequence of the recombinant factor VIII polypeptide comprises at least a substitution or substitution set at amino acid position(s) 1148, 85/708/1148, 798, 706, 171, 335, 63, 344, 428, 879, 59, 429, 205, 1264, 797, 1248, 873, 792, 708, 57, 96, 362, 1292, 346, 433, 65, 795, 684/798, 353, 332, 1259, 356, 436, 1245, 1145, 716, 56, 773, 734, 358, 729, 665, 359, 61, 1250, 726, 37/96/171/429/708/799/1148, 171/429/732/1148, 96/171, 171/919/1151, 171/792/797/799/1148, 171/569/1148, 96/171/429/792/797/799, 171/429/708/1422, 171/429, 321/362/732/797/799, 171/1100, 708/1148/1186, 429/724/732/1148, 171/362/795/797/799, 171/429/732, 96/171/643/797/799, 171/792/797/799, 503, 171/732, 1148/1193, 96/171/362/429/732/795/797, 334, 732, 351, 1377, 999, 306, 349, 257, 646, 435, or 58, wherein the positions are relative to the reference sequence corresponding to SEQ ID NO: 2.

In some embodiments, the amino acid sequence of the recombinant factor VIII polypeptide comprises at least a substitution or substitution set forth in Tables 4.1, 4.2, 5.1, 5.2, 6.1, 6.2, 7.1, 7.2, 8.1, 8.2, 9.1, 9.2, 10.1, 10.2, 11.1, and 15.1, wherein the amino acid positions are relative to the reference sequence corresponding to SEQ ID NO: 2.

In some embodiments, the recombinant factor VIII polypeptide comprises an amino acid sequence having at least 70%, 75%, 80%, 81%, 82%, 83%, 84%, 85%, 86%, 87%, 88%, 89%, 90%, 91%, 92%, 93%, 94%, 95%, 96%, 97%, 98%, or 99% or more sequence identity to the reference sequence corresponding to residues 20-1457 of SEQ ID NO: 252, 504, 706, 840, 1200, 1514, or 2072, or to the reference sequence corresponding to SEQ ID NO: 252, 504, 706, 840, 1200, 1514, or 2072.

In some embodiments, the recombinant factor VIII polypeptide comprises an amino acid sequence having at least 70%, 75%, 80%, 81%, 82%, 83%, 84%, 85%, 86%, 87%, 88%, 89%, 90%, 91%, 92%, 93%, 94%, 95%, 96%, 97%, 98%, or 99% or more sequence identity to a reference sequence corresponding to residues 20-1457 of an even-numbered SEQ ID NO. of SEQ ID NOs. 4-2346 and 2360-2870, or to a reference sequence corresponding to an even-numbered SEQ ID NO. of SEQ ID NOs. 4-2346 and 2350-2870.

In some embodiments, the recombinant factor VIII polypeptide comprises an amino acid sequence having at least 70%, 75%, 80%, 81%, 82%, 83%, 84%, 85%, 86%, 87%, 88%, 89%, 90%, 91%, 92%, 93%, 94%, 95%, 96%, 97%, 98%, or 99% or more sequence identity to the reference sequence corresponding to residues 20-1457 of SEQ ID NO: 252, 504, 706, 840, 1200, 1514, or 2072, or to the reference sequence corresponding to SEQ ID NO: 252, 504, 706, 840, 1200, 1514, or 2072, wherein the amino acid sequence comprises one or more substitutions relative to the reference sequence corresponding to residues 20-1457 of SEQ ID NO: 252, 504, 706, 840, 1200, 1514, or 2072, or relative to the reference sequence corresponding to SEQ ID NO: 252, 504, 706, 840, 1200, 1514, or 2072.

In some embodiments of the foregoing, the amino acid sequence of the recombinant factor VIII polypeptide comprises at least a substitution at amino acid position 13, 14, 15, 16, 17, 18, 19, 20, 21, 24, 37, 56, 57, 58, 59, 61, 63, 64, 65, 85, 96, 104, 110, 119, 127, 132, 158, 160, 164, 167, 168, 171, 175, 178, 186, 187, 205, 210, 211, 212, 215, 225, 246, 257, 260, 265, 281, 292, 294, 301, 303, 304, 306, 308, 310, 311, 312, 318, 320, 321, 325, 328, 332, 334, 335, 344, 346, 347, 349, 351, 353, 354, 355, 356, 358, 359, 362, 370, 387, 388, 390, 392, 393, 397, 407, 428, 429, 433, 435, 436, 481, 487, 488, 489, 491, 498, 499, 503, 514, 527, 533, 534, 535, 536, 538, 543, 550, 551, 555, 558, 569, 580, 581, 584, 610, 611, 612, 613, 614, 615, 616, 617, 618, 619, 620, 621, 622, 643, 646, 650, 665, 667, 676, 680, 681, 682, 683, 684, 686, 693, 696, 698, 700, 706, 707, 708, 712, 716, 724, 726, 729, 731, 732, 734, 735, 736, 737, 738, 739, 740, 744, 746, 747, 748, 749, 750, 751, 752, 753, 754, 755, 756, 757, 759, 770, 773, 775, 785, 786, 787, 790, 791, 792, 793, 794, 795, 797, 798, 799, 800, 801, 802, 805, 806, 807, 808, 809, 810, 811, 812, 815, 816, 817, 818, 819, 821, 822, 824, 825, 826, 828, 830, 831, 836, 837, 838, 839, 840, 841, 844, 866, 873, 879, 892, 896, 897, 903, 907, 914, 916, 917, 918, 919, 921, 922, 923, 924, 926, 929, 938, 939, 945, 948, 951, 968, 999, 1037, 1040, 1041, 1042, 1043, 1049, 1050, 1051, 1053, 1054, 1076, 1078, 1080, 1084, 1088, 1089, 1092, 1093, 1094, 1095, 1098, 1099, 1100, 1101, 1104, 1108, 1109, 1113, 1114, 1116, 1127, 1129, 1138, 1145, 1148, 1151, 1156, 1158, 1160, 1186, 1192, 1193, 1194, 1205, 1210, 1218, 1220, 1221, 1222, 1224, 1229, 1245, 1248, 1250, 1259, 1264, 1269, 1270, 1274, 1281, 1283, 1292, 1297, 1339, 1352, 1373, 1377, 1378, 1380, 1389, 1414, 1422, 1427, 1428, 1439, 1440, 1442, or 1452, or combinations thereof, wherein the amino acid positions are relative to the reference sequence corresponding to SEQ ID NO: 252, 504, 706, 840, 1200, 1514, or 2072.

In some embodiments, the amino acid sequence of the recombinant factor VIII polypeptide comprises at least a substitution or an amino acid residue 13G/Y, 14G/M/V, 15G/S, 16A/D/G/P/V, 17S/V, 18L/R, 19G, 20L, 21M, 24V, 37H, 56E/P, 57A/L/S/T/V, 58K, 59D/G/L/M/Q/R/S/V/W, 61Q/S, 63C/E/F/G/H/I/K/L/M/N/Q/R/S/T/W/Y, 64D/G, 65A, 85Q, 96I/L/R/V/W, 104S, 110H, 119G/V, 127V, 132P/R, 158G, 160E/T, 164E, 167G/H/R, 168G/R, 171A/D/E/F/H/K/N/M/P/Q/R/S/T/V/W/Y, 175A/G/H, 178C/M/Q/V/W, 186A, 187C/G, 205R, 210P, 211G/K/P/Q/R, 212A/F/G/L/K/M/N/P/R/S/V/Y, 215E/P/S, 225R, 246D, 257I, 260E/K/T, 265R/S/W, 281A/S, 292D, 294G, 301K/M, 303G/T, 304A/E/G, 306A/D/G/P/S/T, 308P, 310G/V, 311E/G, 312L, 318C/G/K/L/M/Q/R/T/V, 320G/I/L/P/Q/R/S/T/V, 321Y, 325Y, 328G/Q/S, 332A/F/G/H/N/P/Q/R/T/V/W/Y, 334G, 335E/P, 344A/E/L, 346R/V, 347N, 349G, 351S, 353S, 354R, 355C, 356L/N, 358K/S, 359E/G, 362E/G/N/P/T/V/W, 370A, 387A/G/I/L/Q/R/S/T/V/W, 388A/P, 390A/E/G/L/M/P/S/T/V, 392A, 393A/F/G/H/M/Q/R/W/Y, 397G/N, 407V, 428F, 429L/S, 433A/G, 435Q, 436D/G, 481V, 487N, 488G, 489E/F/Q/R, 491E, 498A, 499C/L, 503S, 514L, 527A, 533R/V/Y, 534I/F/L/M, 535C/G/N, 536A/G/K/L/M/P/R/W, 538V, 543G/L/P/W, 550G, 551W, 555E/G/R/T, 558A/E/G/L/P/Q/R/S/T/V/W, 569T, 580S/Y, 581K/V, 584A, 610H/K/L/R/S/T, 611D/E, 612G/S/T, 613S, 614A/F/H/I/N/T, 615D/N, 616D/E/G/Q/W, 617D/E/G/N, 618C/E/G/P/Y, 619E, 620N/P, 621K/M/R/T/W, 622D, 643N, 646I, 650M, 665G, 667I, 676Q, 680L/R, 681A/C/E/F/G/L/N/R/Q/S/T/W, 682R, 683W, 684G/K, 686W, 693T, 696E, 698D/S/W/Y, 700A/E/Q/T/V, 706A/G/M/Y, 707H, 708A/K/T/V, 712G/L/M/Q/S/T/Y, 716K/L/W, 724I, 726A/L, 729T, 731I/L/R, 732C/D/E/G/L/R/W/K/M/Q/S/T/V/Y, 734I/R/S, 735D/S/V, 736F/G, 737F/G/K/L/M/N/S/V, 738A/D/E/F/H/K/N/P/R/S/T/Y, 739G/P/R, 740G, 744A/R, 746P, 747D, 748F/G/K/N/S/V, 749A/C/D/F/G/I/K/L/M/P/Q/R/S/T/V/Y, 750A/D/G/K/M/N/P/Q/R/S/T/V/W, 751A/D/E/Y, 752D/E/F/G/L/M/Q/R/S/V, 753D, 754W, 755G/L/P/Q/S/Y, 756G/P/R/T, 757A/G/K/T, 759S, 770W, 773H, 775E/G/K/N/P/S/V, 785V, 786C/D/L, 787W, 790A/G/I/M/P/W, 791M/T, 792H/W/Y, 793G, 794R, 795C/N/S/T, 797C/G/P/Q/T/W/Y, 798C/R/T, 799A/E/G/M/P/Q/R/S/V, 800C/G/I/L/M/N/P/Q/R/T/V/W/Y, 801A/G/I/R, 802S/V, 805C/G/H/K/L/M/P/Q/R/S/V/W, 806E/G/R/S, 807C/L/P/R, 808K/S, 809A/C/G/I/K/L/N/Q/R/S/T/V/W, 810V, 811F, 812G/I/V, 815I, 816V/W, 817A/D, 818A/E/G/P/S/V/W, 819V, 821Q, 822S/T, 824W, 825L, 826G, 828K, 830M/V, 831W, 836G/R/V/W, 837G, 838G/T, 839A/G/K/P, 840G, 841W, 844H, 866A, 873L/Q/T, 879Q, 892T, 896L/V, 897G/K/R, 903E, 907D, 914V, 916C/E/I/K/L/M/Q/T, 917G, 918M/S, 919G, 921R/W, 922W, 923M, 924I/M/P/Q/W, 926T/V, 929A/Q/V, 938R/V, 939E/S, 945K, 948S, 951V, 968A/P, 999R, 1037H/M/N/P, 1040E, 1041G/H/N, 1042A/E/T, 1043E/R, 1049G/W, 1050E/P/Q/S/T, 1051E/H/K/R/S/W, 1053A/R, 1054C, 1076G/S, 1078S, 1080G/V, 1084L/N, 1088G/S, 1089F/S, 1092G, 1093A/E/G/M/N/P/Q/R/S/T/V/W/Y, 1094S, 1095G, 1098G, 1099G, 1100I/S, 1101G, 1104C/E, 1108M/S/V/W, 1109G, 1113F, 1114A/M, 1116A/G/I/L/R, 1127L/V, 1129P, 1138Q, 1145R, 1148A/C/E/L/M/N/Q/R/S, 1151S, 1156D/G/N/R, 1158N, 1160R, 1186E, 1192D, 1193A/E/G/I/L, 1194P, 1205L, 1210M/T/V/W, 1218A/G/L/M/P, 1220D/E/L/T, 1221G/S/T/V, 1222F, 1224P, 1229D, 1245R, 1248A/C/D/E/F/G/H/I/K/M/P/Q/R/S/T/W/Y, 1250A/P/S/T, 1259T, 1264L/R/W, 1269F, 1270V, 1274N/V, 1281H/K/P/Q/R/S, 1283E/H/L/S/T, 1292Y, 1297A, 1339C, 1352E, 1373S, 1377A/C/D/E/G/H/I/M/P/R/S/V/T/Y, 1378A/C/D/E, 1380A/G/W, 1389W, 1414A/C/D/E/G/H/N/P/T/W, 1422I, 1427Y, 1428G, 1439A/E/H/L/S/T, 1440A/C/E/G/S/T, 1442T, or 1452L, or combinations thereof, wherein the amino acid positions are relative to the reference sequence corresponding to SEQ ID NO: 252, 504, 706, 840, 1200, 1514, or 2072.

In some embodiments, the amino acid sequence of the recombinant factor VIII polypeptide comprises at least a substitution at amino acid position 63, 171, 212, 260, 306, 317, 320, 332, 387, 390, 393, 534, 558, 681, 732, 738, 749, 750, 775, 1148, 1248, 1377, or 1439, or combinations thereof, wherein the amino acid positions are relative to the reference sequence corresponding to SEQ ID NO: 252, 504, 706, 840, 1200, 1514, or 2072.

In some embodiments, the amino acid sequence of the recombinant factor VIII polypeptide comprises at least a substitution or amino acid residue 63C/E/F/G/H/I/K/L/M/N/Q/R/S/T/W/Y, 171A/D/E/F/H/K/N/M/P/Q/R/S/T/V/W/Y, 212A/F/G/L/K/M/N/P/R/S/V/Y, 260E/K/T, 306A/D/G/P/S/T, 318C/G/K/L/M/Q/R/T/V, 320G/I/L/P/Q/R/S/T/V, 332A/F/G/H/N/P/Q/R/T/V/W/Y, 387A/G/I/L/Q/R/S/T/V/W, 390A/E/G/L/M/P/S/T/V, 393A/F/G/H/M/Q/R/W/Y, 499C/L, 534I/F/L/M, 558A/E/G/L/P/Q/R/S/T/V/W, 681A/C/E/F/G/L/N/R/Q/S/T/W, 700A/E/Q/T/V, 732C/D/E/G/L/R/W/K/M/Q/S/T/V/Y, 738A/D/E/F/H/K/N/P/R/S/T/Y, 749A/C/D/F/G/I/K/L/M/P/Q/R/S/T/V/Y, 750A/D/G/K/M/N/P/Q/R/S/T/V/W, 752D/E/F/G/L/M/Q/R/S/V, 775E/G/K/N/P/S/V, 800C/G/I/L/M/N/P/Q/R/T/V/W/Y, 805C/G/H/K/L/M/P/Q/R/S/V/W, 809A/C/G/I/K/L/N/Q/R/S/T/V/W, 1093A/E/G/M/N/P/Q/R/S/T/V/W/Y, 1148A/C/E/L/M/N/Q/R/S, 1248A/C/D/E/F/G/H/I/K/M/P/Q/R/S/T/W/Y, 1377A/C/D/E/G/H/I/M/P/R/S/V/T/Y, 1414A/C/D/E/G/H/N/P/T/W, or 1439A/E/H/L/S/T, or combinations thereof, wherein the amino acid positions are relative to the reference sequence corresponding to SEQ ID NO: 252, 504, 706, 840, 1200, 1514, or 2072.

In some embodiments, the recombinant factor VIII comprises an amino acid sequence having at least 70%, 75%, 80%, 81%, 82%, 83%, 84%, 85%, 86%, 87%, 88%, 89%, 90%, 91%, 92%, 93%, 94%, 95%, 96%, 97%, 98%, or 99% or more sequence identity to the reference sequence corresponding to residues 20-1457 of SEQ ID NO: 252, or to the reference sequence corresponding to SEQ ID NO: 252, wherein the amino acid sequence comprises one or more substitutions relative to the reference sequence corresponding to residues 20-1457 of SEQ ID NO: 252, or relative to the reference sequence corresponding to SEQ ID NO: 252.

In some embodiments, the amino acid sequence of the recombinant factor VIII polypeptide comprises at least a substitution or substitution set at amino acid position(s) 1380, 388, 1043, 610, 1274, 1220, 1442, 1193, 621, 1054, 755, 1041, 748, 1049, 1221, 1051, 612, 1037, 616, 387, 489, 750, 1224, 618, 1050, 1053, 896, 897, 749, 260, 481, 613, 756, 63, 265, 752, 1218, 1378, 614, 1440, 1439, 1205, 1222, 1389, 1269, 390, 397/1218, 757, 1373, 346, 63/1248, 349, 306/355/1422, 1422, 306/346/708, 1148, 171/346/1248, 63/797/799/1248/1422, 171/799, 362, 1248, 171/306/346/999/1248, 171/1148, 734/799/1248/1422, 63/732, 63/171/306/346/732, 171, 171/1422, 63/362/732/799/1248, 999/1148/1422, 346/349, 306/349, 63/171/1148/1151, 306, 344/349/354/1250, 1250, 63/306/344/349/354/503/1248, 59/63/332/349/503/706/708, 63/306/344/349/354/706/1248, 59/63, 332/706/708/1248/1250, 503, 59/63/349/354, 59/349/708, 59/306/344/1248, 344/1248/1250, 708, 63/344/349, 59, 63/344/349/354, 706, 59/306/770, 63/347/354/1248, or 354/503, wherein the amino acid positions are relative to the reference sequence corresponding to SEQ ID NO: 252.

In some embodiments, the recombinant factor VIII polypeptide comprises an amino acid sequence having at least 70%, 75%, 80%, 81%, 82%, 83%, 84%, 85%, 86%, 87%, 88%, 89%, 90%, 91%, 92%, 93%, 94%, 95%, 96%, 97%, 98%, or 99% or more sequence identity to the reference sequence corresponding to residues 20-1457 of SEQ ID NO: 504, or to the reference sequence corresponding to SEQ ID NO: 504, wherein the amino acid sequence comprises one or more substitutions relative to the reference sequence corresponding to residues 20-1457 of SEQ ID NO: 504, or relative to the reference sequence corresponding to SEQ ID NO: 504.

In some embodiments, the amino acid sequence of the recombinant factor VIII polypeptide comprises at least a substitution or substitution set at amino acid position(s) 750, 1051, 260, 756, 1193, 332, 897, 387, 1037, 306/749, 1270, 489, 487, 488, 903, 171/1248/1440, 260/1148/1378, 171/260/387/750/1248, 171/260, 171/260/387/750/1051/1148, 171, 750/1051/1148, 171/260/756, 171/1248, 171/1148, 59, 1440, 332/750/1248, 756/1248, 1248, 171/387/1248/1440, 750/1440, 59/171/750, 171/1193/1248, 750/1148/1193/1440, 1148, 171/387/1051, 748, 306, 306/387/390/749/1220, 390, 306/390/1037, 306/612/749, 387/390/749, 63/390/503/749, 1220, 63/306, 749, 306/390/749/1037, 306/749/1220, 306/387/390/612/749/1037, or 328, wherein the amino acid positions are relative to the reference sequence corresponding to SEQ ID NO: 504.

In some embodiments, the recombinant factor VIII polypeptide comprises an amino acid sequence having at least 70%, 75%, 80%, 81%, 82%, 83%, 84%, 85%, 86%, 87%, 88%, 89%, 90%, 91%, 92%, 93%, 94%, 95%, 96%, 97%, 98%, or 99% or more sequence identity to the reference sequence corresponding to residues 20-1457 of SEQ ID NO: 706, or to the reference sequence corresponding to SEQ ID NO: 706, wherein the amino acid sequence comprises one or more substitutions relative to the reference sequence corresponding to residues 20-1457 of SEQ ID NO: 706, or relative to the reference sequence corresponding to SEQ ID NO: 706.

In some embodiments, the amino acid sequence of the recombinant factor VIII polypeptide comprises at least a substitution or substitution set at amino acid position(s) 1051, 1221, 1218, 387, 749/1218, 332, 897, 696, 816, 824, 826, 393, 825, 698, 332/387/390/1148, 328/387/749, 749, 306/328/332/390/1051/1193/1218, 63/306/332/1051/1148, 63/306/332/387/1051, 387/390/1051, 186/306/328/332/390/749/1051/1148/1193, 387/390/756/1051, 306/390/1051, 306/332/390/749, 306/387/390/1148, 306/328/332/387/390/749/1051/1148/1193, 332/387/390/749/1148, 387/390/749, 306/387/749/1051/1148, 387/749, 306/945/1051/1193/1221, 306/390/749/1051, 63/306/332/390, 306/387/390/749/1148/1193/1221, 306/332/387/390/749/1218, 63/306/332/390/1051/1148/1193/1218, 924, 755, 752, 751, 923, 797, 754, 793, 753, 922, 896, 938, 775, 732, 810, 747, 393/407, 819, 787, 802, 811, 392, or 397, wherein the amino acid positions are relative to the reference sequence corresponding to SEQ ID NO: 706.

In some embodiments, the recombinant factor VIII polypeptide comprises an amino acid sequence having at least 70%, 75%, 80%, 81%, 82%, 83%, 84%, 85%, 86%, 87%, 88%, 89%, 90%, 91%, 92%, 93%, 94%, 95%, 96%, 97%, 98%, or 99% or more sequence identity to the reference sequence corresponding to residues 20-1457 of SEQ ID NO: 840, or to the reference sequence corresponding to SEQ ID NO: 840, wherein the amino acid sequence comprises one or more substitutions relative to the reference sequence corresponding to residues 20-1457 of SEQ ID NOs: 840, or relative to the reference sequence corresponding to SEQ ID NO: 840.

In some embodiments, the amino acid sequence of the recombinant factor VIII polypeptide comprises at least a substitution or substitution set at amino acid position(s) 620, 491, 1229, 615, 1283, 822, 328, 212, 818, 555/684, 828, 550, 830, 839, 13, 14, 17, 836, 1210, 1281, 64, 682, 318, 20, 21, 325, 533, 19, 817, 831, 225, 1156, 393/747, 753, 393/732/751, 63/797, 622, 617, 907, 1040, 611, 488, 1192, 1194, 619, 16, 802, 211, 555, 536, 527, 841, 320, 837, 558, 914, 312, 681, 838, 15, 1427, 18, 683, 24, 534, 840, 1160, 551, 393, 393/747/752/811/819/923/1148, 393/751/775, 63/393/732/775/1148, 393/732/1148, 775/1148, 393/752/753/775/793/797, 393/923, 393/923/1148, 393/747/751, 393/747/753/775/1148, 752/923/1051, 63/775, 63/393/752/775/793/1051/1053, 752/753/775/793/819, 63/1051, 63/747/752/753/775/811/819/923/1051, 393/752/787, 751, 63/1148, 63/747/752/753/819/923/1148, 1148, 747/752/753/793/811, 63, 797/1148, 751/752/753/1051, 63/732/1051, 752/797/815/923/1051/1148, or 747/753, wherein the amino acid positions are relative to the reference sequence corresponding to SEQ ID NO: 840.

In some embodiments, the recombinant factor VIII polypeptide of comprises an amino acid sequence having at least 70%, 75%, 80%, 81%, 82%, 83%, 84%, 85%, 86%, 87%, 88%, 89%, 90%, 91%, 92%, 93%, 94%, 95%, 96%, 97%, 98%, or 99% or more sequence identity to the reference sequence corresponding to residues 20-1457 of SEQ ID NO: 1200, or to the reference sequence corresponding to SEQ ID NO: 1200, wherein the amino acid sequence comprises one or more substitutions relative to the reference sequence corresponding to residues 20-1457 of SEQ ID NOs: 1200, or relative to the reference sequence corresponding to SEQ ID NO: 1200.

In some embodiments, the amino acid sequence of the recombinant factor VIII polypeptide comprises at least a substitution or substitution set at amino acid position(s) 681, 622, 617, 558, 536, 211, 320, 839, 797, 533, 751, 303, 951, 164, 818, 807, 799, 735, 794, 926, 755, 921, 801, 1094, 968, 929, 924, 698, 168, 581, 916, 1428, 805, 1114, 311, 737, 693, 809, 757, 1099, 676, 740, 791, 211/536/622/681, 536/558, 211/536/555/617/732, 211/681/732, 211/536/555, 555, 211/536, 211/212, 211/536/617/681/732, 211/536/558/617/681, 555/732, 536/555/558/617/732, 536/617/681/732, 536/555, 536/622/681, 732, 212/320/533/617/681, 211/312/533/534/617/619/681/839, 212, 211/212/320/619/681/839, 797/839, 212/312/320/533/534/617/681, 558/619/681, 320/558/681, 212/320/534/619/681/797/839, 320/558/619/732/839, 211/212/312/534/558/619/681/732/839, 320/534/558/619/732/797, 558/617/619/681, 211/534/558/611/681, 211/320/617/619/681, 558/617/681/797, 320/534/619/732, 211/619/681, 211/212/534/558/619/732/839, 212/320/619/681/797/839, 212/320/611/839, 320/558/619/681, 312/533/617/681/797/839, 558/611/681, 312, 211/320/558/619/681/732/797/839, 320/558/732/797, 533/558/611/619/716/732/839, 312/681, 320/534/619/681, 211/320/533/536/619, 558/617/681/839, 212/320/534/558/681, 533/534/681/839, 212/534/619/681, 617/619/681, 211/212/320/558/617/681, 212/534/558/681/797, 212/320/534, 211/312/619, 212/312/619/839, 210/212/320/558/681, 212/312/534/681/797/839, 211/320/558/839, 294, 759, 538, or 1109, wherein the amino acid positions are relative to the reference sequence corresponding to SEQ ID NO: 1200.

In some embodiments, the recombinant factor VIII polypeptide comprises an amino acid sequence having at least 70%, 75%, 80%, 81%, 82%, 83%, 84%, 85%, 86%, 87%, 88%, 89%, 90%, 91%, 92%, 93%, 94%, 95%, 96%, 97%, 98%, or 99% or more sequence identity to the reference sequence corresponding to residues 20-1457 of SEQ ID NO: 1514, or to the reference sequence corresponding to SEQ ID NO: 1514, wherein the amino acid sequence comprises one or more substitutions relative to the reference sequence corresponding to residues 20-1457 of SEQ ID NOs: 1514, or to the reference sequence corresponding to SEQ ID NO: 1514.

In some embodiments, the amino acid sequence of the recombinant factor VIII polypeptide comprises at least a substitution or substitution set at amino acid position(s) 1042, 1041, 1051, 1283, 1281, 916, 187, 215, 178, 1050, 1210, 104, 615, 610, 536, 618, 621, 614, 110, 301, 1078, 812, 786, 127, 175, 1093, 1088, 650, 535, 806, 738, 700, 132, 1076, 1084, 498, 756, 1108, 746, 160, 543, 310, 584, 918, 1452, 790, 292, 800, 167, 892, 304, 1116, 752, 281, 917, 712, 1080, 806/1352, 1095, 744, 1098, 948, 119, 785, 1113, 667, 158, 580, 707, 731, 1100, 1089, 1092, 939, 1104, 736, 1297, 1127, 686, 1129, 1138, 786/821, 680, 1101, 808, 739, 308, 536/555/619/732/799/805, 555/619/801, 555/619/799, 619/799/801/805, 536/555/619/732/799, 799/805, 801, 246/619/799/801, 536/555/619, 619/839, 619/799/805, 536/801/805, 555/619/799/805, 619/801/805, 555/619, 536/619/801, 801/805, 536/619/799/805, 555/619/732/799/801/805, 749, 616, 611, 370/614, 1156, 1158, 555/681/737/799, 536/619/809/1109, 536/555/619/737/799/809/839/916/921, 916/1109, 536/619/681/799/916/1109, 555/619/681/807/1109, 555/619/681/737/799, 619/681/799/916/1109, 619/681, 555/921, 619/681/916, 555/619/681/799, 555, 732, 619/799, 619, 681, 555/916, 839, or 799, wherein the amino acid positions are relative to the reference sequence corresponding to SEQ ID NO: 1514.

In some embodiments, the recombinant factor VIII polypeptide comprises an amino acid sequence having at least 70%, 75%, 80%, 81%, 82%, 83%, 84%, 85%, 86%, 87%, 88%, 89%, 90%, 91%, 92%, 93%, 94%, 95%, 96%, 97%, 98%, or 99% or more sequence identity to the reference sequence corresponding to residues 20-1457 of SEQ ID NO: 2072, or to the reference sequence corresponding to SEQ ID NO: 2072, wherein the amino acid sequence comprises one or more substitutions relative to the reference sequence corresponding to residues 20-1457 of SEQ ID NO: 2072, or relative to the reference sequence corresponding to SEQ ID NO: 2072.

In some embodiments, the amino acid sequence of the recombinant factor VIII polypeptide comprises at least a substitution or substitution set at amino acid position(s) 167, 167/738/1076/1093, 167/738/752/800/805/808/809/1116, 167/800/809/1076, 167/318, 167/318/800/805/808/809, 167/318/681, 167/318/681/700/805/808/809/1076/1116, 167/318/700, 167, 167/738/1076/1093, 167/738/752/800/805/808/809/1116, 167/800/809/1076, 167/318, 167/318/800/805/808/809, 167/318/681, 167/318/681/700/805/808/809/1076/1116, 167/318/700, 167/318/700/1076, 167/318/700/738/739/1076, 167/681/738/800/808/809, 167/178/700/752/1093, 167/700, 167/700/738/739/1093, 167/700/738/752/1093, 167/700/738/752/805/808/809/1093/1116, 808/809, 1076, 738, 738/808/809, 738/1076, 738/739, 738/739/809, 738/739/809/1093/1116, 738/739/1093, 738/739/752/1093, 738/739/799/800/1093, 738/739/1116, 738/800, 738/1093, 738/752, 738/752/1093, 738/752/799, 738/799/809/1093, 738/799/1093, 739, 739/800/809, 800, 800/805/809, 809, 809/1093, 809/1093/1116, 809/1116, 318, 318/1076, 318/1076/1116, 318/739/805/808/809/1076, 318/499/700/738/809, 318/1093, 318/752/1093, 318/1116, 318/543/739, 318/543/681/752/1116, 318/543/1093, 318/700/738/1093/1414, 681, 681/738, 681/738/739/1093, 681/738/739/752/799/800/809/1116/1339, 681/738/739/1116, 681/738/800, 681/800/805/809, 681/809/1093, 681/752/1093, 681/752/1093, 681/700, 681/700/738/739/1093/1116, 681/700/809, 681/700/1093, 681/700/752/808/809/1093/1116, 681/700/752/1093, 681/700/844, 681/700/805/809, 1093, 1093/1116, 752, 752/1076, 752/800/809, 752/800/1093, 752/809, 752/866, 752/1093, 752/1116, 178/318/739/752/809/1076/1116, 178/318/800/809, 178/318/681/800/808/809/1076, 178/681, 178/681/738/1116, 178/752/809, 178/700/800/805/808/809, 178/805/809/866, 1116, 543, 543/738/739/809, 543/738/809/1093, 543/739/1093, 543/800/1093/1116, 543/809/1093, 543/809/1093/1116, 543/681/738/739/752/809/1093, 543/681/738/739/752/1093, 543/681/739/752/800/805/809/1093, 543/681/700/738/739/800/1093/1116, 543/681/700/738/739/805/808/809, 543/681/700/752, 543/681/700/752/809/1093, 543/1093, 543/700, 543/700/738/739/752/800/1093/1116, 543/700/809, 543/700/1093, 543/700/752, 543/700/752/800/1093, 543/700/752/809/1116, 543/700/752/1093, 700, 700/738/739/800/1093, 700/738/739/799/1093, 700/738/800/1093, 700/738/1116, 700/809, 700/809/1093, 700/1093, 700/1093/1116, 700/752, 700/752/808/809/1093/1116, 700/752/809/1093, 700/752/799/1116, 700/752/805/809/1093, 700/799/800, 700/1116, 700/805/808/809/1076, 514/543/681/805/808/809, 805/808/809, or 805/809/1116, wherein the amino acid positions are relative to the reference sequence corresponding to SEQ ID NO: 2072.

In some embodiments, the recombinant factor VIII polypeptide comprises an amino acid sequence having at least one substitution set forth in Tables 4.1, 4.2, 5.1, 5.2, 6.1, 6.2, 7.1, 7.2, 8.1, 8.2, 9.1, 9.2, 10.1, 10.2, 11.1, and 15.1, wherein the amino acid positions are relative to the reference sequence corresponding to SEQ ID NO: 2, 252, 504, 706, 840, 1200, 1514, or 2072.

In some embodiments, the recombinant factor VIII polypeptide comprises an amino acid sequence having at least a substitution or substitution set of a variant set forth in Tables 4.1, 4.2, 5.1, 5.2, 6.1, 6.2, 7.1, 7.2, 8.1, 8.2, 9.1, 9.2, 10.1, 10.2, 11.1, and 15.1, wherein the amino acid positions are relative to the reference sequence corresponding to SEQ ID NO: 2, 252, 504, 706, 840, 1200, 1514, or 2072.

In some embodiments, the recombinant factor VIII polypeptide comprises an amino acid sequence comprising residues 20-1457 of the sequence of an even numbered SEQ ID NO. set forth in Tables 4.1, 4.2, 5.1, 5.2, 6.1, 6.2, 7.1, 7.2, 8.1, 8.2, 9.1, 9.2, 10.1, 10.2, 11.1, and 15.1, or comprising the sequence of an even numbered SEQ ID NO. set forth in Tables 4.1, 4.2, 5.1, 5.2, 6.1, 6.2, 7.1, 7.2, 8.1, 8.2, 9.1, 9.2, 10.1, 10.2, 11.1, and 15.1.

In some embodiments, the recombinant factor VIII polypeptide comprises an amino acid sequence comprising residues 20-1457 of an even-numbered SEQ ID NO. of SEQ ID NOs: 4-2346 and 2360-2870, or comprising an even-numbered SEQ ID NO. of SEQ ID NOs: 4-2346 and 2350-2870. In some embodiments, the amino acid sequence of the recombinant factor VIII polypeptide optionally includes 1, 2, 3, 4, 5, 6, 7, 8, 9, or up to 10 amino acid substitutions. In some embodiments, the amino acid sequence of the recombinant factor VIII polypeptide optionally includes 1, 2, 3, 4, or 5 amino acid substitutions.

In some embodiments, the recombinant factor VIII polypeptide comprises an amino acid sequence comprising residues 20-1457 of SEQ ID NO: 252, 504, 706, 840, 1200, 1514, or 2072, or comprising SEQ ID NO: 252, 504, 706, 840, 1200, 1514, or 2072. In some embodiments, the amino acid sequence of the recombinant factor VIII polypeptide optionally includes 1, 2, 3, 4, 5, 6, 7, 8, 9, or up to 10 amino acid substitutions. In some embodiments, the amino acid sequence of the recombinant factor VIII polypeptide optionally includes 1, 2, 3, 4, or 5 amino acid substitutions.

In the embodiments herein, the recombinant factor VIII polypeptide has clotting activity. In some embodiments, the recombinant factor VIII polypeptide displays an improved property as compared to a reference factor VIII polypeptide. In some embodiments, the reference factor VIII polypeptide has a sequence corresponding to residues 20-1457 of SEQ ID NO: 2, 252, 504, 706, 840, 1200, 1514, or 2072, or a sequence corresponding to SEQ ID NO: 2, 252, 504, 706, 840, 1200, 1514, or 2072. In some embodiments, the reference factor VIII polypeptide has the sequence corresponding to residues 20-1457 of SEQ ID NO: 2, or the sequence corresponding to SEQ ID NO: 2.

In some embodiments, the recombinant factor VIII polypeptide described herein exhibits at least one improved property selected from: i) increased activity/potency; ii) increased clotting activity/potency; iii) increased stability to plasma or serum; iv) increased thermostability; v) reduced immunogenicity; vi) increased expression and/or secretion from cells that are transfected with an expression vector; vii) increased transduction and/or secretion from cells that are transduced with a gene therapy vector (e.g., AAV, mRNA, or lipid nanoparticle), or a combination of any of i), ii), iii), iv), v), vi), and vii), as compared to reference factor VIII polypeptide having a sequence corresponding to residues 20-1457 of SEQ ID NO: 2, 252, 504, 706, 840, 1200, 1514, or 2072, or having a sequence corresponding to SEQ ID NO: 2, 252, 504, 706, 840, 1200, 1514, or 2072. In some embodiments, the reference factor VIII polypeptide has the sequence corresponding to residues 20-1457 of SEQ ID NO: 2, or has the sequence corresponding to SEQ ID NO: 2.

In some embodiments, the recombinant factor VIII polypeptide comprises a “deimmunized” recombinant factor VIII polypeptide, such as provided in Tables 12-1 to 12-8 and 15-2.

In some further embodiments, the recombinant factor VIII is purified. In a further aspect, the present disclosure also provides functional or biologically active fragments of the recombinant factor VIII polypeptides disclosed herein.

In another aspect, the present disclosure further provides a recombinant polynucleotide encoding at least one recombinant factor VIII polypeptide, or a biologically active fragment thereof, described herein. In some embodiments, the polynucleotide is DNA, RNA, or mRNA.

In some embodiments, the recombinant polynucleotide comprises a polynucleotide sequence having at least 70%, 75%, 80%, 81%, 82%, 83%, 84%, 85%, 86%, 87%, 88%, 89%, 90%, 91%, 92%, 93%, 94%, 95%, 96%, 97%, 98%, or 99% or more sequence identity to a reference polynucleotide sequence corresponding to nucleotide residues 58-4371 of an odd-numbered SEQ ID NO. of SEQ ID NOs: 1-2345 and 2359-2869, or to a reference polynucleotide sequence corresponding to an odd-numbered SEQ ID NO. of SEQ ID NOs: 1-2345 and 2349-2869, wherein the recombinant polynucleotide encodes a recombinant factor VIII polypeptide.

In some embodiments, the recombinant polynucleotide comprises a polynucleotide sequence codon-optimized for expression in a host cell. In some embodiments, the recombinant polynucleotide comprises a polynucleotide sequence codon-optimized for expression in a mammalian cell, particularly a human cell.

In some embodiments, the recombinant polynucleotide comprises a polynucleotide sequence comprising nucleotide residues 58-4371 of an odd-numbered SEQ ID NO. of SEQ ID NOs: 1-2345 and 2359-2869, or comprising an odd-numbered SEQ ID NO. of SEQ ID NOs: 1-2345 and 2349-2869.

In some embodiments, the recombinant polynucleotide comprises a polynucleotide sequence comprising nucleotide residues 58-4371 of SEQ ID NO: 1, 251, 503, 705, 839, 1199, 1513, or 2071, or comprising SEQ ID NO: 1, 251, 503, 705, 839, 1199, 1513, or 2071.

In a further aspect, the present disclosure provides expression vectors comprising the recombinant polynucleotide encoding a recombinant factor VIII polypeptide as described herein. In some embodiments, the recombinant polynucleotide is operably linked to one or more control sequences. In some embodiments, the control sequence comprises a promoter. In some further embodiments, the promoter is a heterologous promoter.

In another aspect, the present disclosure provides a host cell comprising a recombinant polynucleotide encoding a recombinant factor VIII polypeptide. In some embodiments, the host cell is an eukaryotic cell or prokaryotic cell. In some embodiments, the host cell is a mammalian cell, particularly a human cell, such as a cultured human cell. In some embodiments, the host cell is a human stem cell.

In a further aspect, the present disclosure provides methods of producing a recombinant factor VIII polypeptide described herein, comprising culturing a host cell comprising a recombinant polynucleotide encoding a recombinant factor VIII polypeptide disclosed herein, under conditions such that the recombinant factor VIII polypeptide is produced. In some embodiments, the method further comprises the step of recovering the expressed recombinant factor VIII polypeptide, such as from the media or cells. In some embodiments, the method further comprises the step of purifying the expressed recombinant factor VIII polypeptide.

In another aspect, the present disclosure provides compositions comprising at least one recombinant factor VIII polypeptide described herein. In some embodiments, the recombinant factor VIII polypeptides are provided as a pharmaceutical composition. In some embodiments, the pharmaceutical compositions comprises a recombinant factor VIII polypeptide, and a pharmaceutically acceptable carrier and/or excipient. In some embodiments, the pharmaceutical composition is suitable for parenteral injection or infusion, particularly for administration to a human subject.

In some embodiments, the pharmaceutical compositions comprise at least one recombinant polynucleotide encoding a recombinant factor VIII polypeptide, particularly for use in gene therapy. In some embodiments, a composition for gene therapy comprises a recombinant polynucleotide encoding at least one of the recombinant factor VIII polypeptide described herein. In some embodiments, the gene therapy composition comprises a viral vector comprising a recombinant polynucleotide encoding the recombinant factor VIII polypeptide described herein.

In some embodiments, a gene therapy composition comprises a recombinant polynucleotide encoding at least one recombinant factor VIII polypeptides described herein, wherein the polynucleotide is formulated in liposomes, cationic polymers, or with dendrimers or attached to a cell penetrating peptide. In some embodiments, the recombinant polynucleotide of the gene therapy composition is provided as an expression vector for expression in mammalian cells.

In another aspect, the recombinant factor VIII polypeptides and the corresponding recombinant polynucleotides encoding the recombinant factor VIII polypeptides are useful in therapeutic applications for treating conditions or diseases characterized by a deficiency in factor VIII. In some embodiments, a method for treating and/or preventing symptoms of a deficiency in factor VIII in a subject comprises administering to a subject having a deficiency in factor VIII an effective amount of a recombinant factor VIII polypeptide or a pharmaceutical composition comprising the recombinant factor VIII polypeptide. In some embodiments, a method for treating and/or preventing symptoms of deficiency in factor VIII in a subject comprises providing to a subject having a deficiency in factor VIII a gene therapy composition comprising a recombinant polynucleotide encoding a recombinant factor VIII polypeptide described herein.

In a further aspect, the present disclosure also provides use of a recombinant factor VIII polypeptide, or a pharmaceutical thereof, or a gene therapy composition comprising a recombinant polynucleotide encoding the recombinant factor VIII polypeptide, for the treatment of a deficiency in factor VIII in a subject. In some embodiments, use of the recombinant factor VIII polypeptide, or a gene therapy composition comprising a recombinant polynucleotide encoding the recombinant factor VIII polypeptide, is for the preparation of a medicament for treating a deficiency in factor VIII in a subject. In some embodiments, the method or use is for treating hemophilia A in a subject.

### BRIEF DESCRIPTION OF THE DRAWINGS

FIG. 1 shows percentage of 50 donors whose peripheral blood mononuclear cells (PBMCs) generated a CD4 T-cell response when incubated with peptides of recombinant factor VIII in a T-cell assay for examining immuogenicity.

FIG. 2 shows the results of a study in which recombinant factor VIII polypeptides were expressed using either Expi293F or HepG2 cultures and assessed for activity using a chromogenic assay (BIOPHEN™ FVIII:C, Aniara), before or after incubation with Hemophilia A patient plasma (George King Bio-Medical, Inc.). Activities are shown relative to SEQ ID NO: 1514.

FIG. 3 shows the results of a study in which truncated recombinant factor VIII polypeptides were expressed using either Expi293F cultures and assessed for activity using a chromogenic assay (BIOPHEN™ FVIII:C, Aniara). Activities are shown relative to SEQ ID NO: 2 on the left axis. Numbers of amino acids removed relative to SEQ ID NO: 2 are shown on the right axis.

FIG. 4 shows expression of recombinant factor VIII polypeptide variants in Expi293F cells as measured by ELISA.

FIG. 5 shows expression of recombinant factor VIII polypeptide variants in HepG2 cells as measured by ELISA.

FIG. 6 shows activity of recombinant factor VIII polypeptide variants expressed in Expi293F cells as measured by chromogenic assay of the conditioned media, before normalization with protein concentration determined by ELISA.

FIG. 7 shows activity of recombinant factor VIII polypeptide variants expressed in HepG2 cells as measured by chromogenic assay of the conditioned media, before normalization with protein concentration determined by ELISA.

FIG. 8 shows activity of recombinant factor VIII polypeptide variants expressed in Expi293F cells as measured by chromogenic assay of the conditioned media, after normalization with protein concentration determined by ELISA.

FIG. 9 shows activity of recombinant factor VIII polypeptide variants expressed in HepG2 cells as measured by chromogenic assay of the conditioned media, after normalization with protein concentration determined by ELISA.

FIG. 10 shows clotting activity measured using activated partial thromboplastin time assay of condition media of Expi293F cells expressing recombinant factor VIII polypeptide variants.

FIG. 11 shows residual activity as measured using a chromogenic assay of recombinant factor VIII polypeptide variants following exposure of the recombinant factor VIII polypeptide variants to plasma obtained from a Hemophilia A patient for different time periods.

### DESCRIPTION OF THE INVENTION

The present disclosure relates to recombinant factor VIII polypeptide (FVIII) variants and compositions thereof. The recombinant factor VIII polypeptide variants have been engineered to exhibit improved properties, such as improved activity, including blood clotting activity, improved cofactor potency, improved expression, increased thermostability, increased serum stability, increased plasma stability, and/or reduced immunogenicity. The present disclosure also provides use of the compositions comprising the recombinant factor VIII polypeptide variants or the polynucleotides encoding the recombinant factor VIII polypeptide variants for therapeutic applications.

It is to be understood that both the foregoing general description, including the drawings, and the following detailed description are exemplary and explanatory only and are not restrictive of this disclosure.

### Abbreviations and Definitions

Unless defined otherwise, all technical and scientific terms used herein generally have the same meaning as commonly understood by one of ordinary skill in the art to which this invention pertains. Generally, the nomenclature used herein and the laboratory procedures of cell culture, molecular genetics, microbiology, biochemistry, organic chemistry, analytical chemistry and nucleic acid chemistry described below are those well-known and commonly employed in the art. Such techniques are well-known and described in numerous texts and reference works well known to those of skill in the art. Standard techniques, or modifications thereof, are used for chemical syntheses and chemical analyses.

Although any suitable methods and materials similar or equivalent to those described herein find use in the practice of the present invention, some methods and materials are described herein. It is to be understood that this invention is not limited to the particular methodology, protocols, and reagents described, as these may vary, depending upon the context they are used by those of skill in the art. Accordingly, the terms defined immediately below are more fully described by reference to the application as a whole.

Also, as used herein, the singular “a”, “an,” and “the” include the plural references, unless the context clearly indicates otherwise.

Numeric ranges are inclusive of the numbers defining the range. Thus, every numerical range disclosed herein is intended to encompass every narrower numerical range that falls within such broader numerical range, as if such narrower numerical ranges were all expressly written herein. It is also intended that every maximum (or minimum) numerical limitation disclosed herein includes every lower (or higher) numerical limitation, as if such lower (or higher) numerical limitations were expressly written herein.

The term “about” means an acceptable error for a particular value. In some instances “about” means within 0.05%, 0.5%, 1.0%, or 2.0%, of a given value range. In some instances, “about” means within 1, 2, 3, or 4 standard deviations of a given value.

As used herein, the term “comprising” and its cognates are used in their inclusive sense (i.e., equivalent to the term “including” and its corresponding cognates).

It is also to be understood that where descriptions of various embodiments use the term “comprising,” those skilled in the art would understand that in some specific instances, an embodiment can be alternatively described using language “consisting essentially of” or “consisting of.”

Furthermore, the headings provided herein are not limitations of the various aspects or embodiments of the invention which can be had by reference to the application as a whole. Accordingly, the terms defined immediately below are more fully defined by reference to the application as a whole. Nonetheless, in order to facilitate understanding of the invention, a number of terms are defined below.

“ATCC” refers to the American Type Culture Collection whose biorepository collection includes genes and strains.

“NCBI” refers to National Center for Biological Information and the sequence databases provided therein.

1 “Protein,” “polypeptide,” and “peptide” are used interchangeably herein to denote a polymer of at least two amino acids covalently linked by an amide bond, regardless of length or post-translational modification (e.g., glycosylation or phosphorylation).

“Mature protein” or “mature polypeptide” refers to the final processed biological protein or polypeptide or product.

“Pro-protein,” “pro-polypeptide,” or “pro-peptide” refers to a precursor protein, polypeptide, or peptide that is processed by post-translational modification, to form a biologically active protein, polypeptide, or peptide. In some embodiments, the post translational modification is a cleavage reaction to form the protein, polypeptide, or peptide.

“Pre-pro-protein,” “pre-pro-polypeptide,” or “pre-pro-peptide” refers to a precursor protein, polypeptide, or peptide that includes a signal sequence or signal peptide, and which can be processed by posttranslational modification, in particular a cleavage reaction, to generate a pro-protein, pro-polypeptide, or pro-peptide. Generally, a cleavage reaction removes a signal sequence or signal peptide to generate a pro-protein, pro-polypeptide, or pro-peptide.

“Full-length” in context of a protein or polypeptide refers to the protein or polypeptide which is not processed to alter the amino acid sequence of the entire protein or polypeptide. For example, a full-length protein is the entire protein encoded in the corresponding mRNA.

“Amino acids” are referred to herein by either their commonly known three-letter symbols or by the one-letter symbols recommended by IUPAC-IUB Biochemical Nomenclature Commission. The abbreviations used for the genetically encoded amino acids are conventional and are as follows: alanine (Ala or A), arginine (Arg or R), asparagine (Asn or N), aspartate (Asp or D), cysteine (Cys or C), glutamate (Glu or E), glutamine (Gln or Q), glycine (Gly or G), histidine (His or H), isoleucine (Ile or I), leucine (Leu or L), lysine (Lys or K), methionine (Met or M), phenylalanine (Phe or F), proline (Pro or P), serine (Ser or S), threonine (Thr or T), tryptophan (Trp or W), tyrosine (Tyr or Y), and valine (Val or V). When the three-letter abbreviations are used, unless specifically preceded by an “L” or a “D” or clear from the context in which the abbreviation is used, the amino acid may be in either the L- or D-configuration about α-carbon (C_{α}). For example, whereas “Ala” designates alanine without specifying the configuration about the α-carbon, “D-Ala” and “L-Ala” designate D-alanine and L-alanine, respectively. When polypeptide sequences are presented as a string of one-letter or three-letter abbreviations (or mixtures thereof), the sequences are presented in the amino (N) to carboxy (C) direction in accordance with common convention.

“Polynucleotide” is used herein to denote a polymer comprising at least two nucleotides where the nucleotides are either deoxyribonucleotides or ribonucleotides, or mixtures of deoxyribonucleotides and ribonucleotides. In some embodiments, the abbreviations used for the genetically encoding nucleosides are conventional and are as follow: adenosine (A); guanosine (G); cytidine (C); thymidine (T); and uridine (U). Unless specifically delineated, the abbreviated nucleosides may be either ribonucleosides or 2′-deoxyribonucleosides. The nucleosides may be specified as being either ribonucleosides or 2′-deoxyribonucleosides on an individual basis or on an aggregate basis. When nucleic acid sequences are presented as a string of one-letter abbreviations, the sequences are presented in the 5′ to 3′ direction in accordance with common convention, and the phosphates are not indicated. The term “DNA” refers to deoxyribonucleic acid. The term “RNA” refers to ribonucleic acid.

“Engineered,” “recombinant,” “non-naturally occurring,” and “variant,” when used with reference to a cell, a polynucleotide or a polypeptide refer to a material or a material corresponding to the natural or native form of the material that has been modified in a manner that would not otherwise exist in nature or is identical thereto but produced or derived from synthetic materials and/or by manipulation using recombinant techniques.

“Wild-type” and “naturally-occurring” refer to the form found in nature. For example, a wild-type polypeptide or polynucleotide sequence is a sequence present in an organism that can be isolated from a source in nature and which has not been intentionally modified by human manipulation.

“Coding sequence” refers to that part of a nucleic acid (e.g., a gene) that encodes an amino acid sequence of a protein or polypeptide.

1 “Percent (%) sequence identity” is used herein to refer to comparisons among polynucleotides and polypeptides, and are determined by comparing two optimally aligned sequences over a comparison window, wherein the portion of the polynucleotide or polypeptide sequence in the comparison window may comprise additions or deletions (i.e., gaps) as compared to the reference sequence for optimal alignment of the two sequences. The percentage may be calculated by determining the number of positions at which the identical nucleic acid base or amino acid residue occurs in both sequences to yield the number of matched positions, dividing the number of matched positions by the total number of positions in the window of comparison and multiplying the result by 100 to yield the percentage of sequence identity. Alternatively, the percentage may be calculated by determining the number of positions at which either the identical nucleic acid base or amino acid residue occurs in both sequences or a nucleic acid base or amino acid residue is aligned with a gap to yield the number of matched positions, dividing the number of matched positions by the total number of positions in the window of comparison and multiplying the result by 100 to yield the percentage of sequence identity. Those of skill in the art appreciate that there are many established algorithms available to align two sequences. Optimal alignment of sequences for comparison can be conducted, e.g., by the local homology algorithm of Smith and Waterman (Smith and Waterman, Adv. Appl. Math., 1981, 2:482), by the homology alignment algorithm of Needleman and Wunsch (Needleman and Wunsch, J. Mol. Biol., 1970, 48:443), by the search for similarity method of Pearson and Lipman (Pearson and Lipman, Proc. Natl. Acad. Sci. USA, 1988, 85:2444), by computerized implementations of these algorithms (e.g., GAP, BESTFIT, FASTA, and TFASTA in the GCG Wisconsin Software Package), or by visual inspection, as known in the art. Examples of algorithms that are suitable for determining percent sequence identity and sequence similarity include, but are not limited to the BLAST and BLAST 2.0 algorithms, which are described by Altschul et al. (See, Altschul et al., J. Mol. Biol., 1990, 215:403-410; and Altschul et al., 1977, Nucleic Acids Res., 1977, 3389-3402, respectively). Software for performing BLAST analyses is publicly available through the National Center for Biotechnology Information website. This algorithm involves first identifying high scoring sequence pairs (HSPs) by identifying short words of length W in the query sequence, which either match or satisfy some positive-valued threshold score T when aligned with a word of the same length in a database sequence. T is referred to as, the neighborhood word score threshold (See, Altschul et al., supra). These initial neighborhood word hits act as seeds for initiating searches to find longer HSPs containing them. The word hits are then extended in both directions along each sequence for as far as the cumulative alignment score can be increased. Cumulative scores are calculated using, for nucleotide sequences, the parameters M (reward score for a pair of matching residues; always >0) and N (penalty score for mismatching residues; always <0). For amino acid sequences, a scoring matrix is used to calculate the cumulative score. Extension of the word hits in each direction are halted when: the cumulative alignment score falls off by the quantity X from its maximum achieved value; the cumulative score goes to zero or below, due to the accumulation of one or more negative-scoring residue alignments; or the end of either sequence is reached. The BLAST algorithm parameters W, T, and X determine the sensitivity and speed of the alignment. The BLASTN program (for nucleotide sequences) uses as defaults a wordlength (W) of 11, an expectation (E) of 10, M=5, N=−4, and a comparison of both strands. For amino acid sequences, the BLASTP program uses as defaults a wordlength (W) of 3, an expectation (E) of 10, and the BLOSUM62 scoring matrix (See, Henikoff and Henikoff, Proc. Natl. Acad. Sci. USA, 1989, 89:10915). Exemplary determination of sequence alignment and % sequence identity can employ the BESTFIT or GAP programs in the GCG Wisconsin Software package (Accelrys, Madison WI), using default parameters provided.

“Reference sequence” refers to a defined sequence used as a basis for a sequence comparison. A reference sequence may be a subset of a larger sequence, for example, a segment of a full-length gene or polypeptide sequence. Generally, a reference sequence is at least 20 nucleotide or amino acid residues in length, at least 25 residues in length, at least 50 residues in length, at least 100 residues in length or the full length of the nucleic acid or polypeptide. Since two polynucleotides or polypeptides may each (1) comprise a sequence (i.e., a portion of the complete sequence) that is similar between the two sequences, and (2) may further comprise a sequence that is divergent between the two sequences, sequence comparisons between two (or more) polynucleotides or polypeptide are typically performed by comparing sequences of the two polynucleotides or polypeptides over a “comparison window” to identify and compare local regions of sequence similarity. In some embodiments, a “reference sequence” can be based on a primary amino acid sequence, where the reference sequence is a sequence that can have one or more changes in the primary sequence. “Comparison window” refers to a conceptual segment of at least about 20 contiguous nucleotide positions or amino acids residues wherein a sequence may be compared to a reference sequence of at least 20 contiguous nucleotides or amino acids and wherein the portion of the sequence in the comparison window may comprise additions or deletions (i.e., gaps) of 20 percent or less as compared to the reference sequence (which does not comprise additions or deletions) for optimal alignment of the two sequences. The comparison window can be longer than 20 contiguous residues, and includes, optionally 30, 40, 50, 100, or longer windows.

“Corresponding to”, “reference to” or “relative to” when used in the context of the numbering of a given amino acid or polynucleotide sequence refers to the numbering of the residues of a specified reference sequence when the given amino acid or polynucleotide sequence is compared to the reference sequence. In other words, the residue number or residue position of a given polymer is designated with respect to the reference sequence rather than by the actual numerical position of the residue within the given amino acid or polynucleotide sequence. For example, a given amino acid sequence, such as that of a recombinant factor VIII polypeptide, can be aligned to a reference sequence by introducing gaps to optimize residue matches between the two sequences. In these cases, although the gaps are present, the numbering of the residue in the given amino acid or polynucleotide sequence is made with respect to the reference sequence to which it has been aligned.

“Mutation” refers to the alteration of a nucleic acid sequence. In some embodiments, mutations result in changes to the encoded polypeptide sequence (i.e., as compared to the original sequence without the mutation). In some embodiments, the mutation comprises a substitution, such that a different amino acid is produced. In some alternative embodiments, the mutation comprises an addition, such that an amino acid is added (e.g., insertion) to the original polypeptide sequence. In some further embodiments, the mutation comprises a deletion, such that an amino acid is deleted from the original polypeptide sequence. Any number of mutations may be present in a given sequence.

“Amino acid difference” or “residue difference” refers to a difference in the amino acid residue at a position of a polypeptide sequence relative to the amino acid residue at a corresponding position in a reference sequence. The positions of amino acid differences generally are referred to herein as “Xn,” where n refers to the corresponding position in the reference sequence upon which the residue difference is based. For example, a “residue difference at position X59 as compared to SEQ ID NO: 2” refers to a difference of the amino acid residue at the polypeptide position corresponding to position 59 of SEQ ID NO: 2. Thus, if the reference polypeptide of SEQ ID NO: 2 has a phenylalanine at position 59, then a “residue difference at position X59 as compared to SEQ ID NO: 2” an amino acid substitution of any residue other than phenylalanine at the position of the polypeptide corresponding to position 59 of SEQ ID NO: 2. In some instances herein, the specific amino acid residue difference at a position is indicated as “XnY” where “Xn” specified the corresponding position as described above, and “Y” is the single letter identifier of the amino acid found in the engineered polypeptide (i.e., the different residue than in the reference polypeptide). In some instances (e.g., as shown in in Tables 4.1, 4.2, 5.1, 5.2, 6.1, 6.2, 7.1, 7.2, 8.1, 8.2, 9.1, 9.2, 10.1, 10.2, 11.1, and 15.1), the present disclosure also provides specific amino acid differences denoted by the conventional notation “AnB”, where A is the single letter identifier of the residue in the reference sequence, “n” is the number of the residue position in the reference sequence, and B is the single letter identifier of the residue substitution in the sequence of the engineered polypeptide. In some embodiments, the amino acid difference, e.g., a substitution, is denoted by the abbreviation “nB,” without the identifier for the residue in the reference sequence. In some embodiments, the phrase “an amino acid residue nB” denotes the presence of the amino residue in the engineered polypeptide, which may or may not be a substitution in context of a reference polypeptide.

In some instances, a polypeptide of the present disclosure can include one or more amino acid residue differences relative to a reference sequence, which is indicated by a list of the specified positions where residue differences are present relative to the reference sequence, as described below. In some embodiments, where more than one amino acid can be used in a specific residue position of a polypeptide, the various amino acid residues that can be used are separated by a “/” (e.g., X61Q/X61S or X61Q/S or 61Q/S).

“Amino acid substitution set” and “substitution set” refer to a group of amino acid substitutions within a polypeptide sequence. In some embodiments, substitution sets comprise 2, 3, 4, 5, 6, 7, 8, 9, 10, 11, 12, 13, 14, 15, or more amino acid substitutions. In some embodiments, a substitution set refers to the set of amino acid substitutions that is present in any of the variant polypeptides listed in any of the Tables in the Examples. In substitution sets, the individual substitutions are separated by a semicolon (“;”; e.g., Q96R;L171P or abbreviated 96R;171P) or slash (“/”; e.g., Q96R/L171P or abbreviated 96R/171P). In some embodiments, the “substitution” comprises the deletion of an amino acid, and can be denoted by “−” symbol.

“Conservative amino acid substitution” refers to a substitution of a residue with a different residue having a similar side chain, and thus typically involves substitution of the amino acid in the polypeptide with amino acids within the same or similar defined class of amino acids. By way of example and not limitation, an amino acid with an aliphatic side chain may be substituted with another aliphatic amino acid (e.g., alanine, valine, leucine, and isoleucine); an amino acid with hydroxyl side chain is substituted with another amino acid with a hydroxyl side chain (e.g., serine and threonine); an amino acids having aromatic side chains is substituted with another amino acid having an aromatic side chain (e.g., phenylalanine, tyrosine, tryptophan, and histidine); an amino acid with a basic side chain is substituted with another amino acid with a basis side chain (e.g., lysine and arginine); an amino acid with an acidic side chain is substituted with another amino acid with an acidic side chain (e.g., aspartic acid or glutamic acid); and/or a hydrophobic or hydrophilic amino acid is replaced with another hydrophobic or hydrophilic amino acid, respectively.

“Non-conservative substitution” refers to substitution of an amino acid in the polypeptide with an amino acid with significantly differing side chain properties. Non-conservative substitutions may use amino acids between, rather than within, the defined groups and affects (a) the structure of the peptide backbone in the area of the substitution (e.g., proline for glycine) (b) the charge or hydrophobicity, or (c) the bulk of the side chain. By way of example and not limitation, an exemplary non-conservative substitution can be an acidic amino acid substituted with a basic or aliphatic amino acid; an aromatic amino acid substituted with a small amino acid; and a hydrophilic amino acid substituted with a hydrophobic amino acid.

“Deletion” refers to modification to the polypeptide by removal of one or more amino acids from the reference polypeptide. Deletions can comprise removal of 1 or more amino acids, 2 or more amino acids, 5 or more amino acids, 10 or more amino acids, 15 or more amino acids, or 20 or more amino acids, up to 10% of the total number of amino acids, or up to 20% of the total number of amino acids making up the reference polypeptide while retaining activity and/or retaining the improved properties of an engineered polypeptide. Deletions can be directed to the internal portions and/or terminal portions of the polypeptide. In various embodiments, the deletion can comprise a continuous segment or can be discontinuous.

“Insertion” refers to modification to the polypeptide by addition of one or more amino acids from the reference polypeptide. Insertions can be in the internal portions of the polypeptide, or to the carboxy or amino terminus. Insertions as used herein include fusion proteins as is known in the art. The insertion can be a contiguous segment of amino acids or separated by one or more of the amino acids in the naturally occurring polypeptide.

“Functional fragment” or a “biologically active fragment” used interchangeably herein refers to a polypeptide that has an amino-terminal and/or carboxy-terminal deletion(s) and/or internal deletions, but where the remaining amino acid sequence is identical to the corresponding positions in the sequence to which it is being compared (e.g., a full-length recombinant factor VIII of the present disclosure) and that retains substantially all of the activity of the full-length polypeptide. In some embodiments, “functional fragment” or a “biologically active fragment” encompasses recombinant factor VIII processed into polypeptides by natural biological processes, such as cleavage into polypeptide fragments that form dimers and activated trimers.

“Isolated polypeptide” refers to a polypeptide which is substantially separated from other contaminants that naturally accompany it (e.g., protein, lipids, and polynucleotides). The term embraces polypeptides which have been removed or purified from their naturally-occurring environment or expression system (e.g., host cell or in vitro synthesis). The recombinant factor VIII polypeptides may be present within a cell, present in the cellular medium, or prepared in various forms, such as lysates or isolated preparations. As such, in some embodiments, the recombinant factor VIII polypeptides can be an isolated polypeptide.

“Substantially pure polypeptide” refers to a composition in which the polypeptide species is the predominant species present (i.e., on a molar or weight basis it is more abundant than any other individual macromolecular species in the composition), and is generally a substantially purified composition when the object species comprises at least about 50 percent of the macromolecular species present by mole or % weight. Generally, a substantially pure factor VIII composition comprises about 60% or more, about 70% or more, about 80% or more, about 90% or more, about 95% or more, and about 98% or more of all macromolecular species by mole or % weight present in the composition. In some embodiments, the object species is purified to essential homogeneity (i.e., contaminant species cannot be detected in the composition by conventional detection methods) wherein the composition consists essentially of a single macromolecular species. Solvent species, small molecules (<500 Daltons), and elemental ion species are not considered macromolecular species. In some embodiments, the isolated recombinant factor VIII polypeptides are substantially pure polypeptide compositions.

“Improved protein property” refers to a recombinant factor VIII polypeptide that exhibits an improvement in any protein property as compared to a reference factor VIII polypeptide and/or as a wild-type factor VIII polypeptide or another recombinant factor VIII polypeptide. Improved properties include, but are not limited to, such properties as increased gene expression, increased protein production, increased thermoactivity, increased thermostability, increased activity at various pH levels, increased stability, increased cofactor potency, increased specific activity, increased resistance to substrate and/or product inhibition, increased chemical stability, improved solvent stability, increased tolerance to acidic, neutral, or basic pH, increased tolerance to proteolytic activity (i.e., reduced sensitivity to proteolysis), reduced aggregation, increased solubility, reduced immunogenicity, increased secretion from polypeptide producing cells, etc.

“Increased activity”, “increased cofactor potency”, or “enhanced activity” refers to an improved property of the recombinant factor VIII polypeptides, which can be represented by an increase in specific activity (e.g., product produced/time/weight protein in combination with factor IX (FIX)) or an increase in percent conversion of the substrate to the product (e.g., percent conversion of starting amount of substrate to product in a specified time period using a specified amount of factor VIII in combination with FIX) as compared to the reference factor VIII polypeptide. Exemplary methods to determine protein activity are provided in the Examples. Any property relating to protein activity may be affected. Improvements in protein activity can be from about 1.1 fold the protein activity of the corresponding wild-type protein or a parent protein or polypeptide, to as much as 2-fold, 5-fold, 10-fold, 20-fold, 25-fold, 50-fold, 75-fold, 100-fold, 150-fold, 200-fold or more activity than the naturally occurring factor VIII or another recombinant factor VIII from which the factor VIII polypeptides were derived (e.g., B-domain deleted factor VIII).

“Improved tolerance to basic pH” means that a recombinant factor VIII according to the invention will have increased stability (e.g., higher retained activity at >pH 7 after exposure to basic pH for a specified period of time (1 hour, up to 24 hours)) as compared to a reference factor VIII or another recombinant factor VIII polypeptide.

“Physiological pH” as used herein means the pH range generally found in a subject's (e.g., human) blood. In some embodiments, “physiological pH” is about pH 7 to about 7.4.

“Basic pH” (e.g., used with reference to improved stability to basic pH conditions or increased tolerance to basic pH) means a pH range >pH 7, e.g., about 7 to 11.

“Acidic pH” (e.g., used with reference to improved stability to acidic pH conditions or increased tolerance to acidic pH) means a pH range of pH 7>, e.g., about 1.5 to 4.5.

“Deimmunized” as used herein, refers to the manipulation of a protein sequence to create a variant that is predicted to be not as immunogenic as the wild-type or reference protein. In some embodiments, the predicted deimmunization is complete, in that the variant protein is predicted to not stimulate an immune response in patients to whom the variant protein is administered. This response can be measured by various methods including but not limited to, the presence or abundance of anti-drug antibodies, the presence or abundance of neutralizing antibodies, the presence of an anaphylactic response, peptide presentation on major histocompatibility complex-II (MHC-II) proteins, or the prevalence or intensity of cytokine release upon administration of the protein. In some embodiments, the variant protein is less immunogenic than the wild-type or reference protein. In some embodiments, deimmunization involves modifications to subsequences of proteins (e.g., epitopes) that are recognized by human leukocyte antigen (HLA) receptors. In some embodiments, these epitopes are removed by changing their amino acid sequences to produce a deimmunized variant protein in which such subsequences are no longer recognized by the HLA receptors. In some other embodiments, these epitopes retain binding affinity to HLA receptors, but are not presented. In some embodiments, the deimmunized protein shows lower levels of response in biochemical and cell-biological predictors of human immunological responses including dendritic-cell T-cell activation assays, or (HLA) peptide binding assays. In some embodiments, these epitopes are removed by changing their amino acid sequence to produce a deimmunized variant protein in which the epitopes are no longer recognized by T-cell receptors. In still other embodiments the deimmunized protein induces anergy in its corresponding T-cells, activates T regulatory cells, or results in clonal deletion of recognizing B-cells. In some embodiments, a total immunogenicity score (TIS) reflects the overall predicted immunogenicity of the variant (i.e., a higher score indicates a higher level of predicted immunogenicity).

“Hybridization stringency” relates to hybridization conditions, such as washing conditions, in the hybridization of nucleic acids. Generally, hybridization reactions are performed under conditions of lower stringency, followed by washes of varying but higher stringency (see, e.g., Sambrook et al., Molecular Cloning: A Laboratory Manual, Cold Spring Harbor Laboratory Press, 2001). The term “moderately stringent hybridization” refers to conditions that permit target-DNA to bind a complementary nucleic acid that has about 60% identity, preferably about 75% identity, about 85% identity to the target DNA, with greater than about 90% identity to target-polynucleotide. Exemplary moderately stringent conditions are conditions equivalent to hybridization in 50% formamide, 5×Denhart's solution, 5×SSPE, 0.2% SDS at 42° C., followed by washing in 0.2×SSPE, 0.2% SDS, at 42° C. “High stringency hybridization” refers generally to conditions that are about 10° C. or less from the thermal melting temperature Tₘ as determined under the solution condition for a defined polynucleotide sequence. In some embodiments, a high stringency condition refers to conditions that permit hybridization of only those nucleic acid sequences that form stable hybrids in 0.018M NaCl at 65° C. (i.e., if a hybrid is not stable in 0.018M NaCl at 65° C., it will not be stable under high stringency conditions, as contemplated herein). High stringency conditions can be provided, for example, by hybridization in conditions equivalent to 50% formamide, 5×Denhart's solution, 5×SSPE, 0.2% SDS at 42° C., followed by washing in 0.1×SSPE, and 0.1% SDS at 65° C. Another high stringency condition is hybridizing in conditions equivalent to hybridizing in 5×SSC containing 0.1% (w:v) SDS at 65° C. and washing in 0.1×SSC containing 0.1% SDS at 65° C. Other high stringency hybridization conditions, as well as moderately stringent conditions, are described in the references cited herein.

“Codon optimized” refers to changes in the codons of the polynucleotide encoding a protein such that the encoded protein is more efficiently expressed in the organism and/or cells of interest. Although the genetic code is degenerate in that most amino acids are represented by several codons, called “synonyms” or “synonymous” codons, it is well known that codon usage by particular organisms is nonrandom and biased towards particular codon triplets. This codon usage bias may be higher in reference to a given gene, genes of common function or ancestral origin, highly expressed proteins versus low copy number proteins, and the aggregate protein coding regions of an organism's genome. In some embodiments, the polynucleotides encoding the factor VIII polypeptides may be codon optimized for optimal production from the host organism(s) and/or cell type(s) selected for expression, accounting for GC content, cryptic splice sites, transcription termination signals, motifs that may affect RNA stability, and nucleic acid secondary structures, as well as any other factors of interest.

“Control sequence” refers herein to include all components, which are necessary or advantageous for the expression of a polynucleotide and/or polypeptide of the present application. Each control sequence may be native or foreign to the nucleic acid sequence encoding the polypeptide. Such control sequences include, but are not limited to, a leader, polyadenylation sequence, pro-peptide sequence, promoter sequence, signal peptide sequence, initiation sequence and transcription terminator. In some instances, at a minimum the control sequences include a promoter, and transcriptional and translational stop signals.

“Operably linked” is defined herein as a configuration in which a control sequence is appropriately placed (i.e., in a functional relationship) at a position relative to a polynucleotide of interest such that the control sequence directs or regulates the expression of the polynucleotide and/or polypeptide of interest.

“Promoter sequence” refers to a nucleic acid sequence that is recognized by a host cell for expression of a polynucleotide of interest, such as a coding sequence. The promoter sequence contains transcriptional control sequences, which mediate the expression of a polynucleotide of interest. The promoter may be any nucleic acid sequence which shows transcriptional activity in the host cell of choice including mutant, truncated, and hybrid promoters, and may be obtained from genes encoding extracellular or intracellular polypeptides either homologous or heterologous to the host cell.

“Heterologous” or “recombinant” refers to the relationship between two or more nucleic acid or protein sequences (e.g., a promoter sequence, signal peptide, terminator sequence, etc.) that are derived from different sources and are not associated in nature.

“Suitable reaction conditions” refers to those conditions in the reaction solution (e.g., ranges of protein loading, temperature, pH, buffers, co-solvents, etc.) under which a factor VIII polypeptide of the present disclosure is capable of acting as a cofactor for factor IX to convert a substrate to the desired product compound. Exemplary “suitable reaction conditions” are provided in the present application and illustrated in the Examples.

“Protein loading” refers to the concentration or amount of a component in a reaction mixture at the start of the reaction. “Substrate” in the context of a reaction process involving factor VIII refers to the compound or molecule acted on by the factor VIII-factor IX polypeptide complex.

“Product” in the context of an enzymatic conversion process refers to the compound or molecule resulting from the action of the factor VIII-factor IX polypeptide complex on a substrate.

“Culturing” as used herein refers to the growing of a population of microbial, mammalian, or other suitable cells under any suitable conditions (e.g., using a liquid, gel or solid medium).

“Vector” refers to a polynucleotide construct for introducing a polynucleotide sequence into a cell. In some embodiments, the vector is an expression vector having a polynucleotide of interest operably linked to a suitable control sequence capable of effecting the expression in a suitable host of the polypeptide encoded in the polynucleotide sequence of interest. In some embodiments, an “expression vector” has a promoter sequence operably linked to the polynucleotide sequence (e.g., transgene) to drive expression in a host cell, and in some embodiments, also comprises a transcription terminator sequence.

“Gene therapy vector” as used herein refers to vehicles or carriers suitable for delivery of polynucleotide sequences to cells. In some embodiments, the vectors encapsulate genes (e.g., therapeutic genes) or polynucleotide sequences for delivery to cells or tissues, including but not limited to adenovirus (AV), adeno-associated virus (AAV), lentivirus (LV), and non-viral vectors, such as liposomes. It is not intended that the present invention be limited to any specific gene therapy vector, as any vehicle suitable for a given setting finds use herein. The gene therapy vector may be designed to deliver genes to a specific species or host, or may find more general applicability.

“Expression” as used herein includes any step involved in the production of the polypeptide including, but not limited to, transcription, post-transcriptional modification, translation, and post-translational modification. In some embodiments, the term also encompasses secretion of the polypeptide from a cell.

“Produces” as used herein refers to the production of proteins and/or other compounds by cells. It is intended that the term encompass any step involved in the production of polypeptides including, but not limited to, transcription, post-transcriptional modification, translation, and post-translational modification. In some embodiments, the term also encompasses secretion of the polypeptide from a cell.

“Host cell” and “host strain” refer to suitable hosts for expression vectors comprising a polynucleotide provided herein (e.g., the polynucleotides encoding the recombinant factor VIII variants). In some embodiments, the host cells are prokaryotic or eukaryotic cells that have been transformed or transfected with vectors constructed using recombinant DNA techniques as known in the art.

“Analogue” as used herein means a polypeptide having more than 70% sequence identity but less than 100% sequence identity (e.g., more than 75%, 78%, 80%, 83%, 85%, 88%, 90%, 91%, 92%, 93%, 94%, 95%, 96%, 97%, 98%, 99% sequence identity) with a reference polypeptide. In some embodiments, “analogues” means polypeptides that contain one or more non-naturally occurring amino acid residues including, but not limited, to homoarginine, ornithine and norvaline, as well as naturally occurring amino acids. In some embodiments, analogues also include one or more D-amino acid residues and non-peptide linkages between two or more amino acid residues.

“Therapeutic” as used herein refers to a compound or composition having beneficial or desirable medical effects administered to a subject who shows signs or symptoms of a disease or condition or pathology.

“Pharmaceutical composition” as used herein refers to a composition suitable for pharmaceutical use in a mammalian subject (e.g., human) comprising a pharmaceutically effective amount of a recombinant factor VIII polypeptide and an acceptable carrier and/or excipient.

“Composition” and “formulation” encompass products comprising at least one recombinant factor VIII of the present disclosure, intended for any suitable use (e.g., pharmaceutical compositions, etc.).

“Gene therapy” refers to the delivery of a gene, polydeoxyribonucleotide, or polynucleotide sequence(s) with a gene therapy vector to cells or tissues for the modification of those cells or tissues for the treatment or prevention of a disease. Gene therapy may include replacing a mutated gene that causes disease with a healthy copy of the gene; inactivating, or “knocking out,” a mutated gene that is functioning improperly; or providing a functional copy of a gene sufficient to treat and/or prevent the disease or condition. In some embodiments, gene therapy is used in the treatment of disease in patients.

“mRNA therapy” refers to the delivery of an mRNA polyribonucleotide to cells or tissues for the modification of those cells or tissues for the treatment or prevention of a disease. In some embodiments, the mRNA polynucleotides for delivery to cells or tissue, are formulated, for instance, but not limited to, in liposomes. In some embodiments, mRNA therapy is used in the treatment of a disease in patients.

“Cell therapy” refers to the delivery of living cells that have been modified exogenously to patients to provide a missing gene for the treatment or prevention of a disease. The modified cells are then reintroduced into the body.

“Effective amount” means an amount sufficient to produce the desired result. One of general skill in the art may determine what the effective amount by using routine experimentation.

“Subject” encompasses mammals such as humans, non-human primates, livestock, companion animals, and laboratory animals (e.g., rodents and lagamorphs). It is intended that the term encompass females as well as males.

“Patient” means any subject that is being assessed for, treated for, or is experiencing disease. In some embodiment, the patient is a human patient.

“Infant” refers to a child in the period of the first month afterbirth to approximately one (1) year of age. As used herein, the term “newborn” refers to child in the period from birth to the 28th day of life. The term “premature infant” refers to an infant born after the twentieth completed week of gestation, yet before full term, generally weighing ˜500 to ˜2499 grams at birth. A “very low birth weight infant” is an infant weighing less than 1500 g at birth.

“Child” refers to a person who has not attained the legal age for consent to treatment or research procedures. In some embodiments, the term refers to a person between the time of birth and adolescence.

“Adult” refers to a person who has attained legal age for the relevant jurisdiction (e.g., 18 years of age in the United States). In some embodiments, the term refers to any fully grown, mature organism. In some embodiments, the term “young adult” refers to a person less than 18 years of age, but who has reached sexual maturity.

“Administration” and “administering” a composition mean providing a composition of the present invention to a subject (e.g., to a person suffering from the effects of deficiency in factor VIII, such as hemophilia A).

“Pharmaceutically acceptable” means a material that can be administered to a subject without causing any undesirable biological effects or interacting in a deleterious manner with any of the components in which it is contained and that possesses the desired biological activity.

“Excipient” refers to any pharmaceutically acceptable additive, carrier, diluent, adjuvant, or other ingredient, other than the active pharmaceutical ingredient (API; e.g., the recombinant factor VIII polypeptides of the present disclosure). Excipients are typically included for formulation and/or administration purposes.

“Carrier” when used in reference to a pharmaceutical composition means any of the standard pharmaceutical carrier, buffers, and excipients, such as stabilizers, preservatives, and adjuvants.

“Therapeutically effective amount” when used in reference to symptoms of disease/condition refers to the amount and/or concentration of a compound (e.g., recombinant factor VIII polypeptides) that ameliorates, attenuates, or eliminates one or more symptom of a disease/condition or prevents or delays the onset of symptom(s). A “therapeutically effective amount” when used in reference to a disease or condition refers to the amount and/or concentration of a composition (e.g., recombinant factor VIII polypeptides) that ameliorates, attenuates, or eliminates the disease/condition. In some embodiments, the term is use in reference to the amount of a composition that elicits the biological (e.g., medical) response by a tissue, system, or animal subject that is sought by the researcher, physician, veterinarian, or other clinician.

“Treating” or “treatment” of a disease, disorder, or syndrome, as used herein, includes (i) preventing the disease, disorder, or syndrome from occurring in a subject, i.e., causing the clinical symptoms of the disease, disorder, or syndrome not to develop in an animal that may be exposed to or predisposed to the disease, disorder, or syndrome but does not yet experience or display symptoms of the disease, disorder, or syndrome; (ii) inhibiting the disease, disorder, or syndrome, i.e., arresting its development; and (iii) relieving the disease, disorder, or syndrome, i.e., causing regression of the disease, disorder, or syndrome. As such, the terms “treating,” “treat” and “treatment” encompass preventative (e.g., prophylactic), as well as palliative treatment. As is known in the art, adjustments for systemic versus localized delivery, age, body weight, general health, sex, diet, time of administration, drug interaction and the severity of the condition may be necessary, and will be ascertainable by one of ordinary skill in the art.

### Recombinant Factor VIII Polypeptides

As discussed above, blood clotting factor VIII (FVIII) is the cofactor to the activated clotting factor IX (e.g., FIXa), which in its proteolytically activated form interacts with FIXa to form a tight noncovalent complex that binds to and activates factor X (FX). Hemophilia A is a deficiency in factor VIII caused by missing or defective FVIII protein. Treatment of hemophilia A patients may include enzyme replacement therapy with preparations of factor VIII protein. Standard enzyme replacement therapy uses recombinantly expressed forms of the wild-type human factor VIII or B-domain deleted form of human factor VIII (e.g., XYNTHA® antihemophilic factor (recombinant); Wyeth Pharmaceuticals LLC). It is known that intravenously administered FVIII circulates in the bloodstream and restores blood clotting functions. However, these drugs do not completely relieve patient symptoms, as additional doses are needed after trauma or before surgery. In addition, the protein is relatively unstable and is cleared from the bloodstream within days. Thus, issues remain with available enzyme replacement treatments. In addition, patients may develop an immune response (IgG and IgE antibodies targeting the administered drug), and suffer severe allergic (anaphylactic) reactions, severe infusion reactions, and even death.

The recombinant factor VIII polypeptides, or biologically active fragments thereof disclosed herein, are intended to provide more stable and efficacious proteins suitable for treatment of hemophilia A, yet with reduced side effects and improved outcomes, as compared to currently available treatments. In some embodiments, the recombinant factor VIII polypeptides are characterized by increased stability in blood (pH 7.4), which the enzyme encounters upon introduction into the bloodstream and where the enzyme is active during therapy. The recombinant factor VIII polypeptides also display, among other properties, improved clotting function, and in some embodiments, reduced predicted immunogenicity (deimmunized).

The recombinant factor VIII polypeptides of the present disclosure are derived from a recombinant factor VIII with a deletion of the B-domain (SEQ ID NO: 2; see, e.g., US Patent Publication. No. 2017/0360900), where the B-domain deleted factor VIII is based on the naturally occurring factor VIII amino acid sequence. In brief, for purposes of explanation and not limitation, the factor VIII polypeptide is composed of five domains within the polypeptide: the signal peptide, the A1 domain, the A2 domain, the B domain, and the Light Chain domain. The recombinant factor VIII with a deletion of the B-domain represented by SEQ ID NO: 2 has identical signal peptide, A1, A2, and Light Chain domains to the wild-type human factor VIII, but has reduced the B domain to fourteen residues. In some embodiments, in the present disclosure, when a particular factor VIII variant is referred to by reference to amino acid differences in particular amino acids residues in the sequence of a B-domain deleted factor VIII or reference factor VIII polypeptide, it is to be understood that variants of another factor VIII polypeptide modified in the equivalent position(s) (as determined from the optional amino acid sequence alignment between the respective amino acid sequences) are encompassed herein.

Furthermore, the present disclosure also provides biologically active fragments of the recombinant factor VIII polypeptides variants disclosed herein. It is understood that factor VIII is biologically processed into polypeptide fragments. By way of example and not limitation, a factor VIII with the B-domain deletion may be processed by removal of signal sequence (e.g., amino acid residues 1-19) to yield a processed factor VIII (e.g., amino acid residues of about 20-1457). The factor VIII may next be processed to produce a polypeptide encoding dimerized human factor VIII (or variant thereof), the cleavage or cleavages occurring between residues 760 to 773 of SEQ ID NO: 2 and the polypeptide being devoid of some or all of an amino acid sequence corresponding to amino acids 760 to 773 of SEQ ID NO: 2. The factor VIII may be further processed to produce a polypeptide encoding trimerized human factor VIII (or variant thereof), the additional cleavage occurring between residues 391 and 392 of SEQ ID NO: 2 and the polypeptide being devoid of some or all of an amino acid sequence corresponding to amino acids 760 to 773 of SEQ ID NO: 2. Recombinant factor VIII polypeptide variants may or may not be processed the same as the B-domain deleted factor VIII having the naturally occurring sequences.

Accordingly, a reference to “biologically active” or “functionally active” fragment of the recombinant factor VIII polypeptide herein is meant to include the polypeptides formed by natural biological processing upon expression in a mammalian cell or patient, or upon administration of the recombinant factor VIII polypeptide to a patient, whether the “biologically active” or “functionally active” fragments are products from processing similar to the B-domain deleted factor VIII having the naturally occurring sequence or fragments that are the result of processing different from that of the B-domain deleted factor VIII having the naturally occurring sequence.

In one aspect, the present disclosure provides a recombinant factor VIII polypeptide, or a biologically active fragment thereof, comprising an amino acid sequence having at least 70%, 75%, 80%, 81%, 82%, 83%, 84%, 85%, 86%, 87%, 88%, 89%, 90%, 91%, 92%, 93%, 94%, 95%, 96%, 97%, 98%, or 99% sequence identity to a reference sequence corresponding to residues 20-1457 of SEQ ID NO: 2, 252, 504, 706, 840, 1200, 1514, or 2072, or to a reference sequence corresponding to SEQ ID NO: 2, 252, 504, 706, 840, 1200, 1514, or 2072, wherein the amino acid sequence comprises one or more substitutions relative to the reference sequence.

In some embodiments, the recombinant factor VIII polypeptide, or a biologically active fragment thereof, comprises an amino acid sequence having at least 70%, 75%, 80%, 81%, 82%, 83%, 84%, 85%, 86%, 87%, 88%, 89%, 90%, 91%, 92%, 93%, 94%, 95%, 96%, 97%, 98%, or 99% or more sequence identity to a reference sequence corresponding to residues 20-1457 of SEQ ID NO: 2, 252, 504, 706, 840, 1200, 1514, or 2072, or to a reference sequence corresponding to SEQ ID NO: 2, 252, 504, 706, 840, 1200, 1514, or 2072, wherein the amino acid sequence comprises one or more substitutions relative to the reference sequence corresponding to residues 20-1457 of SEQ ID NO: 2, 252, 504, 706, 840, 1200, 1514, or 2072, or relative to the reference sequence corresponding to SEQ ID NO: 2, 252, 504, 706, 840, 1200, 1514, or 2072.

In some embodiments, the recombinant factor VIII polypeptide, or a biologically active fragment thereof, comprises an amino acid sequence having at least 70%, 75%, 80%, 81%, 82%, 83%, 84%, 85%, 86%, 87%, 88%, 89%, 90%, 91%, 92%, 93%, 94%, 95%, 96%, 97%, 98%, or 99% or more sequence identity to the reference sequence corresponding to residues 20-1457 of SEQ ID NO: 2 or to the reference sequence corresponding to SEQ ID NO: 2, wherein the amino acid sequence comprises one or more substitutions relative to the reference sequence corresponding to residues 20-1457 of SEQ ID NO: 2, or relative to the reference sequence corresponding to SEQ ID NO: 2.

In some embodiments, the recombinant factor VIII polypeptide, or biologically active fragment thereof, comprises an amino acid sequence having at least 70%, 75%, 80%, 81%, 82%, 83%, 84%, 85%, 86%, 87%, 88%, 89%, 90%, 91%, 92%, 93%, 94%, 95%, 96%, 97%, 98%, or 99% or more sequence identity to the reference sequence corresponding to residues 20-1457 of SEQ ID NO: 252, 504, 706, 840, 1200, 1514, or 2072, or to the reference sequence corresponding to SEQ ID NO: 2, 252, 504, 706, 840, 1200, 1514, or 2072, wherein the amino acid sequence comprises one or more substitutions relative to the reference sequence corresponding to residues 20-1457 of SEQ ID NO: 2, or relative to the reference sequence corresponding to SEQ ID NO: 2.

In some embodiments, the recombinant factor VIII polypeptide, or biologically active fragment thereof, comprises an amino acid sequence having at least 70%, 75%, 80%, 81%, 82%, 83%, 84%, 85%, 86%, 87%, 88%, 89%, 90%, 91%, 92%, 93%, 94%, 95%, 96%, 97%, 98%, or 99% or more sequence identity to a reference sequence corresponding to residues 20-1457 of an even-numbered SEQ ID NO. of SEQ ID NOs. 4-2346 and 2360-2870, or to a reference sequence corresponding to an even-numbered SEQ ID NO. of SEQ ID NOs. 4-2346 and 2350-2870, wherein the amino acid sequence comprises one or more substitutions relative to the reference sequence corresponding to residues 20-1457 of SEQ ID NO: 2 or relative to the reference sequence corresponding to SEQ ID NO: 2.

In some embodiments, the amino acid sequence of the recombinant factor VIII polypeptide comprises at least a substitution at amino acid position 13, 14, 15, 16, 17, 18, 19, 20, 21, 24, 37, 56, 57, 58, 59, 61, 63, 64, 65, 85, 96, 104, 110, 119, 127, 132, 158, 160, 164, 167, 168, 171, 175, 178, 186, 187, 205, 210, 211, 212, 215, 225, 246, 257, 260, 265, 281, 292, 294, 301, 303, 304, 306, 308, 310, 311, 312, 318, 320, 321, 325, 328, 332, 334, 335, 344, 346, 347, 349, 351, 353, 354, 355, 356, 358, 359, 362, 370, 387, 388, 390, 392, 393, 397, 407, 428, 429, 433, 435, 436, 481, 487, 488, 489, 491, 498, 499, 503, 514, 527, 533, 534, 535, 536, 538, 543, 550, 551, 555, 558, 569, 580, 581, 584, 610, 611, 612, 613, 614, 615, 616, 617, 618, 619, 620, 621, 622, 643, 646, 650, 665, 667, 676, 680, 681, 682, 683, 684, 686, 693, 696, 698, 700, 706, 707, 708, 712, 716, 724, 726, 729, 731, 732, 734, 735, 736, 737, 738, 739, 740, 744, 746, 747, 748, 749, 750, 751, 752, 753, 754, 755, 756, 757, 759, 770, 773, 775, 785, 786, 787, 790, 791, 792, 793, 794, 795, 797, 798, 799, 800, 801, 802, 805, 806, 807, 808, 809, 810, 811, 812, 815, 816, 817, 818, 819, 821, 822, 824, 825, 826, 828, 830, 831, 836, 837, 838, 839, 840, 841, 844, 866, 873, 879, 892, 896, 897, 903, 907, 914, 916, 917, 918, 919, 921, 922, 923, 924, 926, 929, 938, 939, 945, 948, 951, 968, 999, 1037, 1040, 1041, 1042, 1043, 1049, 1050, 1051, 1053, 1054, 1076, 1078, 1080, 1084, 1088, 1089, 1092, 1093, 1094, 1095, 1098, 1099, 1100, 1101, 1104, 1108, 1109, 1113, 1114, 1116, 1127, 1129, 1138, 1145, 1148, 1151, 1156, 1158, 1160, 1186, 1192, 1193, 1194, 1205, 1210, 1218, 1220, 1221, 1222, 1224, 1229, 1245, 1248, 1250, 1259, 1264, 1269, 1270, 1274, 1281, 1283, 1292, 1297, 1339, 1352, 1373, 1377, 1378, 1380, 1389, 1414, 1422, 1427, 1428, 1439, 1440, 1442, or 1452, or combinations thereof, wherein the amino acid positions are relative to the reference sequence corresponding to SEQ ID NO: 2.

In some embodiments, the amino acid sequence of the recombinant factor VIII polypeptide comprises at least a substitution or amino acid residue 13G/Y, 14G/M/V, 15G/S, 16A/D/G/P/V, 17S/V, 18L/R, 19G, 20L, 21M, 24V, 37H, 56E/P, 57A/L/S/T/V, 58K, 59D/G/L/M/Q/R/S/V/W, 61Q/S, 63C/E/F/G/H/I/K/L/M/N/Q/R/S/T/W/Y, 64D/G, 65A, 85Q, 96I/L/R/V/W, 104S, 110H, 119G/V, 127V, 132P/R, 158G, 160E/T, 164E, 167G/H/R, 168G/R, 171A/D/E/F/H/K/N/M/P/Q/R/S/T/V/W/Y, 175A/G/H, 178C/M/Q/V/W, 186A, 187C/G, 205R, 210P, 211G/K/P/Q/R, 212A/F/G/K/L/M/N/P/R/S/V/Y, 215E/P/S, 225R, 246D, 257I, 260E/K/T, 265R/S/W, 281A/S, 292D, 294G, 301K/M, 303G/T, 304A/E/G, 306A/D/G/P/S/T, 308P, 310G/V, 311E/G, 312L, 318C/G/K/M/Q/R/T/V, 320G/I/L/P/Q/R/S/T/V, 321Y, 325Y, 328G/Q/S, 332A/F/G/H/N/P/Q/R/T/V/W/Y, 334G, 335E/P, 344A/E/L, 346R/V, 347N, 349G, 351S, 353S, 354R, 355C, 356L/N, 358K/S, 359E/G, 362E/G/N/P/T/V/W, 370A, 387A/G/I/L/Q/R/S/T/V/W, 388A/P, 390A/E/G/L/M/P/S/T/V, 392A, 393A/F/G/H/M/Q/R/W/Y, 397G/N, 407V, 428F, 429L/S, 433A/G, 435Q, 436D/G, 481V, 487N, 488G, 489E/F/Q/R, 491E, 498A, 499C/L, 503S, 514L, 527A, 533R/V/Y, 534F/I/L/M, 535C/G/N, 536A/G/K/L/M/P/R/W, 538V, 543G/L/P/W, 550G, 551W, 555E/G/R/T, 558A/E/G/L/P/Q/R/S/T/V/W, 569T, 580S/Y, 581K/V, 584A, 610H/K/L/R/S/T, 611D/E, 612G/S/T, 613S, 614A/F/H/I/N/T, 615D/N, 616D/E/G/Q/W, 617D/E/G/N, 618C/E/G/P/Y, 619E, 620N/P, 621K/M/R/T/W, 622D, 643N, 646I, 650M, 665G, 667I, 676Q, 680L/R, 681A/C/E/F/G/L/N/R/Q/S/T/W, 682R, 683W, 684G/K, 686W, 693T, 696E, 698D/S/W/Y, 700A/E/Q/T/V, 706A/G/M/Y, 707H, 708A/K/T/V, 712G/L/M/Q/S/T/Y, 716K/L/W, 724I, 726A/L, 729T, 731I/L/R, 732C/D/E/G/L/R/W/K/M/Q/S/T/V/Y, 734I/R/S, 735D/S/V, 736F/G, 737F/G/K/L/M/N/S/V, 738A/D/E/F/H/K/N/P/R/S/T, 739G/P/R, 740G, 744A/R, 746P, 747D, 748F/G/K/N/S/V, 749A/C/D/F/G/I/K/L/M/P/Q/R/S/T/V/Y, 750A/D/G/K/M/N/P/Q/R/S/T/V/W, 751A/D/E/Y, 752D/E/F/G/L/M/Q/R/S/V, 753D, 754W, 755G/L/P/Q/S/Y, 756G/P/R/T, 757A/G/K/T, 759S, 770W, 773H, 775E/G/K/N/P/S/V, 785V, 786C/D/L, 787W, 790A/G/I/M/P/W, 791M/T, 792H/W/Y, 793G, 794R, 795C/N/S/T, 797C/G/P/Q/T/W/Y, 798C/R/T, 799A/E/G/M/P/Q/R/S/V, 800C/G/I/L/M/N/P/Q/R/T/V/W/Y, 801A/G/I/R, 802S/V, 805C/G/H/K/L/M/P/Q/R/S/V/W, 806E/G/R/S, 807C/L/P/R, 808K/S, 809A/C/G/I/K/L/N/Q/R/S/T/V/W, 810V, 811F, 812G/I/V, 815I, 816V/W, 817A/D, 818A/E/G/P/S/V/W, 819V, 821Q, 822S/T, 824W, 825L, 826G, 828K, 830M/V, 831W, 836G/R/V/W, 837G, 838G/T, 839A/G/K/P, 840G, 841W, 844H, 866A, 873L/Q/T, 879Q, 892T, 896L/V, 897G/K/R, 903E, 907D, 914V, 916C/E/I/K/L/M/Q/T, 917G, 918M/S, 919G, 921R/W, 922W, 923M, 924I/M/P/Q/W, 926T/V, 929A/Q/V, 938R/V, 939E/S, 945K, 948S, 951V, 968A/P, 999R, 1037H/M/N/P, 1040E, 1041G/H/N, 1042A/E/T, 1043E/R, 1049G/W, 1050E/P/Q/S/T, 1051E/H/K/R/S/W, 1053A/R, 1054C, 1076G/S, 1078S, 1080G/V, 1084L/N, 1088G/S, 1089F/S, 1092G, 1093A/E/G/M/N/P/Q/R/S/T/V/W/Y, 1094S, 1095G, 1098G, 1099G, 1100I/S, 1101G, 1104C/E, 1108M/S/V/W, 1109G, 1113F, 1114A/M, 1116A/G/I/L/R, 1127L/V, 1129P, 1138Q, 1145R, 1148A/C/E/L/M/N/Q/R/S, 1151S, 1156D/G/N/R, 1158N, 1160R, 1186E, 1192D, 1193A/E/G/I/L, 1194P, 1205L, 1210M/T/V/W, 1218A/G/L/M/P, 1220D/E/L/T, 1221G/S/T/V, 1222F, 1224P, 1229D, 1245R, 1248A/C/D/E/F/G/H/I/K/M/P/Q/R/S/T/W/Y, 1250A/P/S/T, 1259T, 1264L/R/W, 1269F, 1270V, 1274N/V, 1281H/K/P/Q/R/S, 1283E/H/L/S/T, 1292Y, 1297A, 1339C, 1352E, 1373S, 1377A/C/D/E/G/H/I/M/P/R/S/V/T/Y, 1378A/C/D/E, 1380A/G/W, 1389W, 1414A/C/D/E/G/H/N/P/T/W, 1422I, 1427Y, 1428G, 1439A/E/H/L/S/T, 1440A/C/E/G/S/T, 1442T, or 1452L, or combinations thereof, wherein the amino acid positions are relative to the reference sequence corresponding to SEQ ID NO: 2.

In some embodiments, the amino acid sequence of the recombinant factor VIII polypeptide comprises at least a substitution L13G/Y, L14G/M/V, R15G/S, F16A/D/G/P/V, C17S/V, F18L/R, S19G, A20L, T21M, Y24V, Q37H, S56E/P, F57A/L/S/T/V, P58K, F59D/G/L/M/Q/R/S/V/W, T61Q/S, V63C/E/F/G/H/I/K/L/M/N/Q/R/S/T/W/Y, V64D/G, Y65A, P85Q, Q96I/L/R/V/W, V104S, M110H, A119G/V, A127V, E132P/R, Q158G, L160E/T, G164E, A167G/H/R, S168G/R, L171A/D/E/F/H/K/N/M/P/Q/R/S/T/V/W/Y, Y175A/G/H, L178C/M/Q/V/W, D186A, L187C/G, K205R, T210P, L211G/K/P/Q/R, H212A/F/G/L/K/M/N/P/R/S/V/Y, I215E/P/S, K225R, A246D, V257I, S260E/K/T, T265R/S/W, T281A/S, G292D, T294G, R301K/M, A303G/T, S304A/E/G, E306A/D/G/P/S/T, S308P, I310G/V, T311E/G, F312L, L318C/G/K/M/Q/R/T/V, M320G/I/L/P/Q/R/S/T/V, D321Y, F325Y, F328G/Q/S, S332A/F/G/H/N/P/Q/R/T/V/W/Y, H334G, Q335E/P, K344A/E/L, D346R/V, S347N, P349G, E351S, Q353S, L354R, R355C, M356L/N, N358K/S, N359E/G, A362E/G/N/P/T/V/W, T370A, F387A/G/I/L/Q/R/S/T/V/W, 1388A/P, I390A/E/G/L/M/P/S/T/V, S392A, V393A/F/G/H/M/Q/R/W/Y, H397G/N, A407V, S428F, Q429L/S, N433A/G, P435Q, Q436D/G, L481V, Q487N, A488G, S489E/F/Q/R, P491E, G498A, 1499C/L, R503S, V514L, I527A, T533R/V/Y, V534I/F/L/M, T535C/G/N, V536A/G/K/L/M/P/R/W, D538V, S543G/L/P/W, R550G, Y551W, F555E/G/R/T, M558A/E/G/L/P/Q/R/S/T/V/W, P569T, Q580S/Y, R581K/V, Q584A, I610H/K/L/R/S/T, Q611D/E, R612G/S/T, F613S, L614A/F/H/I/N/T, P615D/N, N616D/E/G/Q/W, P617D/E/G/N, A618C/E/G/P/Y, G619E, V620N/P, Q621K/M/R/T/W, L622D, S643N, L646I, L650M, T665G, F667I, T676Q, K680L/R, M681A/C/E/F/G/L/N/R/Q/S/T/W, V682R, Y683W, E684G/K, T686W, S693T, T696E, F698D/S/W/Y, S700A/E/Q/T/V, L706A/G/M/Y, W707H, I708A/K/T/V, H712G/L/M/Q/S/T/Y, F716K/L/W, L724I, K726A/L, S729T, D731I/L/R, K732C/D/E/G/L/R/W/K/M/Q/S/T/V/Y, T734I/R/S, G735D/SN, D736F/G, Y737F/G/K/L/M/N/S/V, Y738A/D/E/F/H/K/N/P/R/S/T, E739G/P/R, D740G, D744A/R, A746P, A747D, Y748F/G/K/N/S/V, L749A/C/D/F/G/I/K/L/M/P/Q/R/S/T/V/Y, L750A/D/G/K/M/N/P/Q/R/S/T/V/W, A751A/D/E/Y, K752D/E/F/G/L/M/Q/R/S/V, N753D, N754W, A755G/L/P/Q/S/Y, I756G/P/R/T, E757A/G/K/T, R759S, R770W, R773H, I775E/G/K/N/P/S/V, E785V, E786C/D/L, I787W, D790A/G/I/M/P/W, D791M/T, T792H/W/Y, I793G, S794R, Y795C/N/S/T, M797C/G/P/Q/T/W/Y, K798C/R/T, K799A/E/G/M/P/Q/R/S/V, E800C/G/I/L/M/N/P/Q/R/T/V/W/Y, D801A/G/I/R, F802SN, Y805C/G/H/K/L/M/P/Q/R/SN/W, D806E/G/R/S, E807C/L/P/R, D808K/S, E809A/C/G/I/K/L/N/Q/R/S/T/V/W, N810V, Q811F, S812G/I/V, S815I, F816V/W, Q817A/D, K818A/E/G/P/S/V/W, K819V, R821Q, H822S/T, F824W, I825L, A826G, V828K, R830M/V, L831W, M836G/R/V/W, S837G, S838G/T, S839A/G/K/P, P840G, H841W, R844H, G866A, Y873L/Q/T, E879Q, V892T, 1896L/V, M897G/K/R, Q903E, P907D, L914V, S916C/E/I/K/L/M/Q/T, Y917G, E918M/S, E919G, Q921R/W, R922W, Q923M, G924I/M/P/Q/W, E926T/V, K929A/Q/V, K938R/V, T939E/S, Q945K, M948S, T951V, L968A/P, Q999R, F1037H/M/N/P, N1040E, Y1041G/H/N, R1042A/E/T, F1043E/R, Y1049G/W, I1050E/P/Q/S/T, M1051E/H/K/R/S/W, T1053A/R, L1054C, E1076G/S, I1078S, S1080G/V, S1084L/N, F1088G/S, T1089F/S, K1092G, K1093A/E/G/M/N/P/Q/R/S/T/V/W/Y, E1094S, E1095G, M1098G, A1099G, L1100I/S, Y1101G, Y1104C/E, F1108M/S/V/W, E1109G, M1113F, L1114A/M, S1116A/G/I/L/R, I1127L/V, E1129P, L1138Q, K1145R, T1148A/C/E/L/M/N/Q/R/S, G1151S, H1156D/G/N/R, R1158N, F1160R, A1186E, P1192D, F1193A/E/G/I/L, S1194P, I1205L, K1210M/T/V/W, F1218A/G/L/M/P, S1220D/E/L/T, L1221G/S/T/V, Y1222F, S1224P, M1229D, T1245R, L1248A/C/D/E/F/G/H/I/K/M/P/Q/R/S/T/W/Y, V1250A/P/S/T, G1259T, I1264L/R/W, I1269F, I1270V, I1274N/V, Y1281H/K/P/Q/R/S, I1283E/H/L/S/T, M1292Y, N1297A, G1339C, K1352E, V1373S, L1377A/C/D/E/G/H/I/M/P/R/S/V/T/Y, T1378A/C/D/E, M1380A/G/W, S1389W, 51414A/C/D/E/G/H/N/P/T/W, L1422I, L1427Y, T1428G, V1439A/E/H/L/S/T, H1440A/C/E/G/S/T, I1442T, or E1452L, or combinations thereof, wherein the amino acid positions are relative to the reference sequence corresponding to SEQ ID NO: 2.

In some embodiments, the amino acid sequence of the recombinant factor VIII polypeptide comprises at least a substitution at amino acid position 63, 171, 212, 260, 306, 317, 320, 332, 387, 390, 393, 534, 558, 681, 732, 738, 749, 750, 775, 1148, 1248, 1377, or 1439, or combinations thereof, wherein the amino acid positions are relative to the reference sequence corresponding to SEQ ID NO: 2.

In some embodiments, the amino acid sequence of the recombinant factor VIII polypeptide comprises at least a substitution or amino acid residue 63C/E/F/G/H/I/K/L/M/N/Q/R/S/T/W/Y, 171A/D/E/F/H/K/N/M/P/Q/R/S/T/V/W/Y, 212A/F/G/L/K/M/N/P/R/S/V/Y, 260E/K/T, 306A/D/G/P/S/T, 318C/G/K/L/M/Q/R/T/V, 320G/I/L/P/Q/R/S/T/V, 332A/F/G/H/N/P/Q/R/T/V/W/Y, 387A/G/I/L/Q/R/S/T/V/W, 390A/E/G/L/M/P/S/T/V, 393A/F/G/H/M/Q/R/W/Y, 499C/L, 534I/F/L/M, 558A/E/G/L/P/Q/R/S/T/V/W, 681A/C/E/F/G/L/N/R/Q/S/T/W, 700A/E/Q/T/V, 732C/D/E/G/L/R/W/K/M/Q/S/T/V/Y, 738A/D/E/F/H/K/N/P/R/S/T/Y, 749A/C/D/F/G/I/K/L/M/P/Q/R/S/T/V/Y, 750A/D/G/K/M/N/P/Q/R/S/T/V/W, 752D/E/F/G/L/M/Q/R/S/V, 775E/G/K/N/P/S/V, 800C/G/I/L/M/N/P/Q/R/T/V/W/Y, 805C/G/H/K/L/M/P/Q/R/S/V/W, 809A/C/G/I/K/L/N/Q/R/S/T/V/W, 1093A/E/G/M/N/P/Q/R/S/T/V/W/Y, 1148A/C/E/L/M/N/Q/R/S, 1248A/C/D/E/F/G/H/I/K/M/P/Q/R/S/T/W/Y, 1377A/C/D/E/G/H/I/M/P/R/S/V/T/Y, 1414A/C/D/E/G/H/N/P/T/W, or 1439A/E/H/L/S/T, or combinations thereof, wherein the amino acid positions are relative to the reference sequence corresponding to SEQ ID NO: 2.

In some embodiments, the amino acid sequence of the recombinant factor VIII polypeptide comprises at least a substitution or amino acid residue 63Q, 171P, 212A, 260T, 306G, 317G, 320R, 332P, 387I, 390A, 393G, 534I, 558W, 681G, 732W, 7338E, 749F, 750D, 775G, 1148N, 1248D, 1377D, or 1439E, or combinations thereof, wherein the amino acid positions are relative to the reference sequence corresponding to SEQ ID NO: 2.

In some embodiments, the amino acid sequence of the recombinant factor VIII polypeptide comprises at least a substitution V63Q, L171P, H212A, S260T, E306G, L317G, M320R, S332P, F387I, 1390A, V393G, V534I, M558W, M681G, K732W, Y738E, L749F, L750D, 1775G, T1148N, L1248D, L1377D, or V1439E, or combinations thereof, wherein the amino acid positions are relative to the reference sequence corresponding to SEQ ID NO:2.

In some embodiments, the amino acid sequence of the recombinant factor VIII polypeptide comprises at least a substitution at position 1377. In some embodiments, the amino acid sequence of the recombinant factor VIII polypeptide comprises at least a substitution 1377D.

In some embodiments, the amino acid sequence of the recombinant factor VIII polypeptide comprises at least a substitution or substitution set at position 1439 or 1377/1439. In some embodiments, the amino acid sequence of the recombinant factor VIII polypeptide comprises at least a substitution or substitution set 1439E or 1377D/1439E.

In some embodiments, the amino acid sequence of the recombinant factor VIII polypeptide comprises at least a substitution or substitution set at position(s) 171, 260, 387, 750, 1248 or 171/260/387/750/1248. In some embodiments, the amino acid sequence of the recombinant factor VIII polypeptide comprises at least a substitution or substitution set 171P, 260T, 387I, 750D, 1248D or 171P/260T/387I/750D/1248D.

In some embodiments, the amino acid sequence of the recombinant factor VIII polypeptide comprises at least a substitution or substitution set at position(s) 306, 332, 390, 749 or 306/332/390/749. In some embodiments, the amino acid sequence of the recombinant factor VIII polypeptide comprises at least a substitution or substitution set 306G, 332P, 390A, 749F or 306G/332P/390A/749F.

In some embodiments, the amino acid sequence of the recombinant factor VIII polypeptide comprises at least a substitution or substitution set at position(s) 63, 393, 732, 775, 1148 or 63/393/732/775/1148. In some embodiments, the amino acid sequence of the recombinant factor VIII polypeptide comprises at least a substitution or substitution set 63Q, 393G, 732W, 775G, 1148N or 63Q/393G/732W/775G/1148N.

In some embodiments, the amino acid sequence of the recombinant factor VIII polypeptide comprises at least a substitution or substitution set at position(s) 212, 320, 534, 558, 681, or 212/320/534/558/681. In some embodiments, the amino acid sequence of the recombinant factor VIII polypeptide comprises at least a substitution or substitution set 212A, 320R, 534I, 558W, 681G, or 212A/320R/534I/558W/681G.

In some embodiments, the amino acid sequence of the recombinant factor VIII polypeptide comprises at least a substitution or substitution set at position(s) 317, 738, or 317/738. In some embodiments, the amino acid sequence of the recombinant factor VIII polypeptide comprises at least a substitution or substitution set 317G, 738E, or 317G/738E.

In some embodiments, the amino acid sequence of the recombinant factor VIII polypeptide comprises at least a substitution or substitution set at amino acid position(s) 1148, 85/708/1148, 798, 706, 171, 335, 63, 344, 428, 879, 59, 429, 205, 1264, 797, 1248, 873, 792, 708, 57, 96, 362, 1292, 346, 433, 65, 795, 684/798, 353, 332, 1259, 356, 436, 1245, 1145, 716, 56, 773, 734, 358, 729, 665, 359, 61, 1250, 726, 37/96/171/429/708/799/1148, 171/429/732/1148, 96/171, 171/919/1151, 171/792/797/799/1148, 171/569/1148, 96/171/429/792/797/799, 171/429/708/1422, 171/429, 321/362/732/797/799, 171/1100, 708/1148/1186, 429/724/732/1148, 171/362/795/797/799, 171/429/732, 96/171/643/797/799, 171/792/797/799, 503, 171/732, 1148/1193, 96/171/362/429/732/795/797, 334, 732, 351, 1377, 999, 306, 349, 257, 646, 435, or 58, wherein the positions are relative to the reference sequence corresponding to SEQ ID NO: 2.

In some embodiments, the amino acid sequence of the recombinant factor VIII polypeptide comprises at least a substitution or substitution set 1148N, 85Q/708T/1148N, 798T, 706G, 706A, 171Q, 335E, 63R, 344A, 428F, 879Q, 59L, 429S, 205R, 1264W, 797P, 1248S, 1264R, 873T, 792Y, 708K, 344L, 57V, 96L, 362W, 63G, 873L, 1292Y, 1148Q, 346V, 797G, 706Y, 1248R, 433A, 65A, 59W, 873Q, 795C, 684K/798R, 57L, 353S, 332Q, 708A, 1259T, 63M, 356L, 1248G, 1248P, 436G, 57A, 59S, 1245R, 59R, 96W, 797Y, 1145R, 436D, 716L, 56E, 773H, 734I, 358S, 335P, 59Q, 729T, 716K, 665G, 362T, 57T, 359G, 61S, 1250A, 362V, 359E, 716W, 332W, 708V, 63H, 797C, 61Q, 356N, 1250S, 798C, 433G, 795N, 96I, 56P, 63N, 792W, 1264L, 1250P, 332P, 59V, 726L, 37H/96R/171P/429L/708T/799R/1148N, 171P, 171P/429L/732R/1148N, 96R/171P, 171P/919G/1151S, 171P/792Y/797Q/799R/1148N, 171P/569T/1148N, 96R/171P/429L/792Y/797T/799R, 171P/429L/708T/1422I, 171P/429L, 321Y/362P/732E/797T/799R, 171P/1100I, 708T/1148N/1186E, 429L/724I/732E/1148N, 171P/362E/795T/797Q/799M, 171P/429L/732E, 96R/171P/643N/797T/799R, 171P/792Y/797T/799M, 503S, 171P/732E, 1148N/1193L, 96R/171P/362E/429L/732R/795T/797Q, 726A, 59M, 734R, 362N, 334G, 732G, 63Q, 351S, 1377D, 795S, 358K, 999R, 1250T, 306G, 349G, 1377G, 332F, 1377E, 257I, 646I, 346R, 435Q, 96V, 1377R, 1377S, 59G, 171M, 792H, 1248D, 306S, 706M, 171H, 1377A, 1377V, 734S, 1148R, 57S, 59D, 344E, 362G, or 58K, wherein the amino acid positions are relative to the reference sequence corresponding to SEQ ID NO: 2.

In some embodiments, the amino acid sequence of the recombinant factor VIII polypeptide comprises at least a substitution or substitution set T1148N, P85Q/1708T/T1148N, K798T, L706G, L706A, L171Q, Q335E, V63R, K344A, S428F, E879Q, F59L, Q429S, K205R, I1264W, M797P, L1248S, I1264R, Y873T, T792Y, I708K, K344L, F57V, Q96L, A362W, V63G, Y873L, M1292Y, T1148Q, D346V, M797G, L706Y, L1248R, N433A, Y65A, F59W, Y873Q, V795C, E684K/K798R, F57L, Q353S, S332Q, I708A, G1259T, V63M, M356L, L1248G, L1248P, Q436G, F57A, F59S, T1245R, F59R, Q96W, M797Y, K1145R, Q436D, F716L, S56E, R773H, T734I, N358S, Q335P, F59Q, S729T, F716K, T665G, A362T, F57T, N359G, T61S, V1250A, A362V, N359E, F716W, S332W, I708V, V63H, M797C, T61Q, M356N, V1250S, K798C, N433G, V795N, Q96I, S56P, V63N, T792W, I1264L, V1250P, S332P, F59V, K726L, Q37H/Q96R/L171P/Q429L/I708T/K799R/T1148N, L171P, L171P/Q429L/K732R/T1148N, Q96R/L171P, L171P/E919G/G1151S, L171P/T792Y/M797Q/K799R/T1148N, L171P/P569T/T1148N, Q96R/L171P/Q429L/T792Y/M797T/K799R, L171P/Q429L/I708T/L1422I, L171P/Q429L, D321Y/A362P/K732E/M797T/K799R, L171P/L1100I, I708T/T1148N/A1186E, Q429L/L724I/K732E/T1148N, L171P/A362E/V795T/M797Q/K799M, L171P/Q429L/K732E, Q96R/L171P/S643N/M797T/K799R, L171P/T792Y/M797T/K799M, R503S, L171P/K732E, T1148N/F1193L, Q96R/L171P/A362E/Q429L/K732R/V795T/M797Q, K726A, F59M, T734R, A362N, H334G, K732G, V63Q, E351S, L1377D, V795S, N358K, Q999R, V1250T, E306G, P349G, L1377G, S332F, L1377E, V257I, L646I, D346R, P435Q, Q96V, L1377R, L1377S, F59G, L171M, T792H, L1248D, E306S, L706M, L171H, L1377A, L1377V, T734S, T1148R, F57S, F59D, K344E, A362G, or P58K, wherein the amino acid positions are relative to the reference sequence corresponding to SEQ ID NO: 2.

In some embodiments, the amino acid sequence of the recombinant factor VIII polypeptide comprises at least a substitution or substitution set at amino acid position(s) 1377/1380, 388/1377, 1043/1377, 610/1377, 1274/1377, 1220/1377, 1377/1442, 1193/1377, 621/1377, 1054/1377, 755/1377, 1041/1377, 748/1377, 1049/1377, 1221/1377, 1051/1377, 612/1377, 1037/1377, 616/1377, 387/1377, 489/1377, 750/1377, 1224/1377, 618/1377, 1050/1377, 1053/1377, 896/1377, 897/1377, 749/1377, 260/1377, 481/1377, 613/1377, 756/1377, 63/1377, 265/1377, 752/1377, 1218/1377, 1377/1378, 614/1377, 1378/1377, 1440/1377, 1377/1440, 1377/1439, 1205/1377, 1222/1377, 1377/1389, 1269/1377, 390/1377, 397/1218/1377, 757/1377, 1373/1377, 346/1377, 63/1248/1377, 349/1377, 306/355/1377/1422, 1377/1422, 306/346/708/1377, 1148/1377, 171/346/1248/1377, 63/797/799/1248/1377/1422, 171/799/1377, 362/1377, 1248/1377, 171/306/346/999/1248/1377, 171/1148/1377, 734/799/1248/1377/1422, 63/732/1377, 63/171/306/346/732/1377, 171/1377, 171/1377/1422, 63/362/732/799/1248/1377, 999/1148/1377/1422, 346/349/1377, 306/349/1377, 63/171/1148/1377/1151, 306/1377, 344/349/354/1250/1377, 1250/1377, 63/306/344/349/354/503/1248/1377, 59/63/332/349/503/706/708/1377, 63/306/344/349/354/706/1248/1377, 59/63/1377, 332/706/708/1248/1250/1377, 503/1377, 59/63/349/354/1377, 59/349/708/1377, 59/306/344/1248/1377, 344/1248/1250/1377, 708/1377, 63/344/349/1377, 59/1377, 63/344/349/354/1377, 706/1377, 59/306/770/1377, 63/347/354/1248/1377, or 354/503/1377, wherein the amino acid positions are relative to the reference sequence corresponding to SEQ ID NO: 2.

In some embodiments, the amino acid sequence of the recombinant factor VIII polypeptide comprises at least a substitution or substitution set 1377D/1380W, 388A/1377D, 1043E/1377D, 1043R/1377D, 610R/1377D, 1274N/1377D, 1220T/1377D, 1377D/1442T, 1193L/1377D, 621T/1377D, 1054C/1377D, 755Q/1377D, 1041G/1377D, 748S/1377D, 1049G/1377D, 1221V/1377D, 1051E/1377D, 612G/1377D, 1037H1/1377D, 616E/1377D, 1041H/1377D, 387S/1377D, 489R/1377D, 621M/1377D, 489Q/1377D, 750G/1377D, 1224P/1377D, 618C/1377D, 616G/1377D, 612T/1377D, 1050Q/1377D, 1053A/1377D, 616W/1377D, 896V/1377D, 489F/1377D, 1050P/1377D, 897G/1377D, 749T/1377D, 1274V/1377D, 1220L/1377D, 260E/1377D, 481V/1377D, 613S/1377D, 387G/1377D, 621R/1377D, 756R/1377D, 63R/1377D, 265S/1377D, 1221T/1377D, 1377D/1380G, 752G/1377D, 748K/1377D, 755L/1377D, 1218A/1377D, 748N/1377D, 1377D/1378E, 614I/1377D, 749F/1377D, 387V/1377D, 750T/1377D, 1193G/1377D, 749V/1377D, 1378A/1377D, 750V/1377D, 387T/1377D, 387Q/1377D, 750A/1377D, 1037N/1377D, 1377D/1440A, 1051S/1377D, 1221G/1377D, 1377D/1378D, 1377D/1440S, 387R/1377D, 1193A/1377D, 387I/1377D, 1218M/1377D, 265W/1377D, 1377D/1440E, 1377D/1439L, 1377D/1440T, 612S/1377D, 1049W/1377D, 1221S/1377D, 1220D/1377D, 749G/1377D, 749S/1377D, 1218G/1377D, 1205L/1377D, 1222F/1377D, 1193E/1377D, 1050S/1377D, 1377D/1389W, 1377D/1439E, 260T/1377D, 1037P/1377D, 1051K/1377D, 1269F/1377D, 265R/1377D, 1051R/1377D, 1218L/1377D, 897R/1377D, 388P/1377D, 748G/1377D, 756P/1377D, 748F/1377D, 390A/1377D, 610L/1377D, 755G/1377D, 749R/1377D, 748V/1377D, 1377D/1380A, 1377D/1440C, 397N/F1218M/1377D, 1037M/1377D, 1218P/1377D, 757K/1377D, 1373S/1377D, 1377D/1440G, 1193I/1377D, 1377D/1439T, 750D/1377D, 390G/1377D, 756G/1377D, 1220E/1377D, 1377D/1378C, 618G/1377D, 346R/1377D, 63Q/1248D/1377D, 349G/1377D, 306G/355C/1377D/1422I, 1377D/1422I, 306G/346R/708T/1377D, 1148N/1377D, 171P/346R/1248D/1377D, 63Q/797T/799R/1248D/1377D/1422I, 171P/799R/1377D, 362P/1377D, 1248D/1377D, 63Q/1377D, 171P/306G/346R/999R/1248D/1377D, 171P/1148N/1377D, 734S/799R/1248D/1377D/1422I, 63Q/732E/1377D, 63Q/171P/306G/346R/732G/1377D, 171P/1377D, 171P/1377D/1422I, 63Q/362P/732G/799R/1248D/1377D, 999R/1148N/1377D/1422I, 346R/349G/1377D, 306G/349G/1377D, 63Q/171P/1148N/1377D/1151S, 306G/1377D, 344E/349G/354R/1250T/1377D, 63G/1377D, 1250T/1377D, 63R/306G/344E/349G/354R/503S/1248R/1377D, 59D/63S/332P/349G/503S/706M/708K/1377D, 63S/306G/344A/349G/354R/706M/1248R/1377D, 1248R/1377D, 59D/63G/1377D, 332P/706M/708K/1248R/1250T/1377D, 503S/1377D, 59D/63R/349G/354R/1377D, 59D/349G/708K/1377D, 59D/306G/344E/1248R/1377D, 344E/1248R/1250T/1377D, 708K/1377D, 63R/344E/349G/1377D, 59D/1377D, 63S/344A/349G/354R/1377D, 706M/1377D, 59D/306G/770W/1377D, 63G/347N/354R/1248R/1377D, or 354R/503S/1377D, wherein the amino acid positions are relative to the reference sequence corresponding to SEQ ID NO: 2.

In some embodiments, the amino acid sequence of the recombinant factor VIII polypeptide comprises at least a substitution or substitution set at amino acid position(s) 750/1377/1439, 1051/1377/1439, 260/1377/1439, 756/1377/1439, 1193/1377/1439, 332/1377/1439, 897/1377/1439, 387/1377/1439, 1037/1377/1439, 306/749/1377/1439, 1270/1377/1439, 489/1377/1439, 487/1377/1439, 488/1377/1439, 903/1377/1439, 171/1248/1377/1439/1440, 260/1148/1377/1378/1439, 171/260/387/750/1248/1377/1439, 171/260/1377/1439, 171/260/387/750/1051/1148/1377/1439, 171/1377/1439, 750/1051/1148/1377/1439, 171/260/756/1377/1439, 171/1248/1377/1439, 171/1148/1377/1439, 59/1377/1439, 1377/1439/1440, 332/750/1248/1377/1439, 756/1248/1377/1439, 1248/1377/1439, 171/387/1248/1377/1440/1439, 750/1377/1439/1440, 59/171/750/1377/1439, 171/1193/1248/1377/1439, 750/1148/1193/1377/1439/1440, 1148/1377/1439, 171/387/1051/1377/1439, 748/1377/1439, 306/1377/1439, 306/387/390/749/1220/1377/1439, 390/1377/1439, 306/390/1037/1377/1439, 306/612/749/1377/1439, 387/390/749/1377/1439, 63/390/503/749/1377/1439, 1220/1377/1439, 63/306/1377/1439, 749/1377/1439, 306/390/749/1037/1377/1439, 306/749/1220/1377/1439, 306/387/390/612/749/1037/1377/1439, or 328/1377/1439, wherein the amino acid positions are relative to the reference sequence corresponding to SEQ ID NO: 2.

In some embodiments, the amino acid sequence of the recombinant factor VIII polypeptide comprises at least a substitution or substitution set 750D/1377D/1439E, 1051K/1377D/1439E, 1051S/1377D/1439E, 260T/1377D/1439E, 756P/1377D/1439E, 1193A/1377D/1439E, 332P/1377D/1439E, 897R/1377D/1439E, 387Q/1377D/1439E, 1037N/1377D/1439E, 387R/1377D/1439E, 306G/749F/1377D/1439E, 1270V/1377D/1439E, 489E/1377D/1439E, 487N/1377D/1439E, 488G/1377D/1439E, 903E/1377D/1439E, 171P/1248D/1377D/1439E/1440T, 260T/1148N/1377D/1378D/1439E, 171P/260T/387I/750D/1248D/1377D/1439E, 171P/260T/1377D/1439E, 171P/260T/387I/750D/1051S/1148N/1377D/1439E, 171P/1377D/1439E, 750D/1051S/1148N/1377D/1439E, 171P/260T/756P/1377D/1439E, 171P/1248D/1377D/1439E, 171P/1148N/1377D/1439E, 387I/1377D/1439E, 59D/1377D/1439E, 1377D/1439E/1440S, 332P/750D/1248D/1377D/1439E, 756P/1248D/1377D/1439E, 1248D/1377D/1439E, 171P/387I/1248D/1377D/1440S/1439E, 750D/1377D/1439E/1440S, 59D/171P/750D/1377D/1439E, 171P/1193A/1248D/1377D/1439E, 750D/1148N/1193A/1377D/1439E/1440S, 1148N/1377D/1439E, 171P/387I I051K/1377D/1439E, 748G/1377D/1439E, 306G/1377D/1439E, 306G/387R/390A/749F/1220D/1377D/1439E, 390A/1377D/1439E, 306G/390A/1037N/1377D/1439E, 306G/612S/749F/1377D/1439E, 387V/390A/749F/1377D/1439E, 63S/390A/503S/749F/1377D/1439E, 387R/390A/749R/1377D/1439E, 1220D/1377D/1439E, 63S/306G/1377D/1439E, 749F/1377D/1439E, 306G/390A/749F/1037N/1377D/1439E, 387V/1377D/1439E, 306G/749F/1220D/1377D/1439E, 306G/387V/390A/612S/749F/1037N/1377D/1439E, or 328S/1377D/1439E wherein the amino acid positions are relative to the reference sequence corresponding to SEQ ID NO: 2.

In some embodiments, the amino acid sequence of the recombinant factor VIII polypeptide comprises at least a substitution or substitution set at amino acid position(s) 171/260/387/750/1051/1248/1377/1439, 171/260/387/750/1221/1248/1377/1439, 171/260/387/750/1218/1248/1377/1439, 171/260/387/750/1248/1377/1439, 171/260/387/749/750/1218/1248/1377/1439, 171/260/332/387/750/1248/1377/1439, 171/260/387/750/897/1248/1377/1439, 171/260/387/696/750/1248/1377/1439, 171/260/387/750/816/1248/1377/1439, 171/260/387/750/824/1248/1377/1439, 171/260/387/750/826/1248/1377/1439, 171/260/387/393/750/1248/1377/1439, 171/260/387/750/825/1248/1377/1439, 171/260/387/698/750/1248/1377/1439, 171/260/332/387/390/750/1148/1248/1377/1439, 171/260/328/387/749/750/1248/1377/1439, 171/260/387/749/750/1248/1377/1439, 171/260/306/328/332/387/390/750/1051/1193/1218/1248/1377/1439, 63/171/260/306/332/387/750/1051/1148/1248/1377/1439, 63/171/260/306/332/387/750/1051/1248/1377/1439, 171/260/387/390/750/1051/1248/1377/1439, 171/186/260/306/328/332/387/390/749/750/1051/1148/1193/1248/1377/1439, 171/260/387/390/750/756/1051/1248/1377/1439, 171/260/306/387/390/750/1051/1248/1377/1439, 171/260/306/332/387/390/749/750/1248/1377/1439, 171/260/306/387/390/750/1148/1248/1377/1439, 171/260/306/328/332/387/390/749/750/1051/1148/1193/1248/1377/1439, 171/260/332/387/390/749/750/1148/1248/1377/1439, 171/260/387/390/749/750/1248/1377/1439, 171/260/306/387/749/750/1051/1148/1248/1377/1439, 171/260/306/387/750/945/1051/1193/1221/1248/1377/1439, 171/260/306/387/390/749/750/1051/1248/1377/1439, 63/171/260/306/332/387/390/750/1248/1377/1439, 171/260/306/387/390/749/750/1148/1193/1221/1248/1377/1439, 171/260/306/332/387/390/749/750/1218/1248/1377/1439, 63/171/260/306/332/387/390/750/1051/1148/1193/1218/1248/1377/1439, 171/260/387/750/924/1248/1377/1439, 171/260/387/750/755/1248/1377/1439, 171/260/387/750/752/1248/1377/1439, 171/260/387/750/751/1248/1377/1439, 171/260/387/750/923/1248/1377/1439, 171/260/387/750/797/1248/1377/1439, 171/260/387/750/754/1248/1377/1439, 171/260/387/750/793/1248/1377/1439, 171/260/387/750/753/1248/1377/1439, 171/260/387/750/922/1248/1377/1439, 171/260/387/750/896/1248/1377/1439, 171/260/387/750/938/1248/1377/1439, 171/260/387/750/775/1248/1377/1439, 171/260/387/750/732/1248/1377/1439, 171/260/387/750/810/1248/1377/1439, 171/260/387/750/747/1248/1377/1439, 171/260/387/393/407/750/1248/1377/1439, 171/260/387/750/819/1248/1377/1439, 171/260/387/750/787/1248/1377/1439, 171/260/387/750/802/1248/1377/1439, 171/260/387/732/750/1248/1377/1439, 171/260/387/750/811/1248/1377/1439, 171/260/387/392/750/1248/1377/1439, or 171/260/387/397/750/1248/1377/1439, wherein the amino acid positions are relative to the reference sequence of SEQ ID NO: 2.

In some embodiments, the amino acid sequence of the recombinant factor VIII polypeptide comprises at least a substitution or substitution set 171P/260T/387I/750D/1051K/1248D/1377D/1439E, 171P/260T/387I/750D/1221G/1248D/1377D/1439E, 171P/260T/387I/750D/1218G/1248D/1377D/1439E, 171P/260T/387R/750D/1248D/1377D/1439E, 171P/260T/387I/750D/1051R/1248D/1377D/1439E, 171P/260T/387I/749F/750D/1218G/1248D/1377D/1439E, 171P/260T/387T/750D/1248D/1377D/1439E, 171P/260T/332P/387I/750D/1248D/1377D/1439E, 171P/260T/387I/750D/897K/1248D/1377D/1439E, 171P/260T/387I/696E/750D/1248D/1377D/1439E, 171P/260T/387I/750D/816W/1248D/1377D/1439E, 171P/260T/387I/750D/824W/1248D/1377D/1439E, 171P/260T/387I/750D/826G/1248D/1377D/1439E, 171P/260T/387I/393W/750D/1248D/1377D/1439E, 171P/260T/387I/750D/825L/1248D/1377D/1439E, 171P/260T/387I/750D/897R/1248D/1377D/1439E, 171P/260T/387I/750D/816V/1248D/1377D/1439E, 171P/260T/387I/698W/750D/1248D/1377D/1439E, 171P/260T/387I/393Q/750D/1248D/1377D/1439E, 171P/260T/332P/387R/390A/750D/1148N/1248D/1377D/1439E, 171P/260T/328S/387R/749F/750D/1248D/1377D/1439E, 171P/260T/387I/749F/750D/1248D/1377D/1439E, 171P/260T/306G/328S/332P/387I/390A/750D/1051R/1193A/1218G/1248D/1377D/1439E, 63Q/171P/260T/306G/332P/387I/750D/1051R/1148N/1248D/1377D/1439E, 63Q/171P/260T/306G/332P/387T/750D/1051K/1248D/1377D/1439E, 171P/260T/387T/390A/750D/1051R/1248D/1377D/1439E, 171P/186A/260T/306G/328S/332P/387I/390A/749F/750D/1051K/1148N/1193A/1248D/1377D/1439E, 171P/260T/387T/390A/750D/756P/1051R/1248D/1377D/1439E, 171P/260T/306G/387I/390A/750D/1051R/1248D/1377D/1439E, 171P/260T/306G/332P/387I/390A/749F/750D/1248D/1377D/1439E, 171P/260T/306G/387R/390A/750D/1148N/1248D/1377D/1439E, 171P/260T/306G/328S/332P/387T/390A/749F/750D/1051R/1148N/1193A/1248D/1377D/1439E, 171P/260T/332P/387R/390A/749F/750D/1148N/1248D/1377D/1439E, 171P/260T/387R/390A/749F/750D/1248D/1377D/1439E, 171P/260T/306G/387T/749F/750D/1051K/1148N/1248D/1377D/1439E, 171P/260T/387R/749F/750D/1248D/1377D/1439E, 171P/260T/306G/387I/750D/945K/1051R/1193A/1221G/1248D/1377D/1439E, 171P/260T/306G/387I/390A/749F/750D/1051K/1248D/1377D/1439E, 63Q/171P/260T/306G/332P/387I/390A/750D/1248D/1377D/1439E, 171P/260T/306G/387T/390A/749F/750D/1148N/1193A/1221G/1248D/1377D/1439E, 171P/260T/306G/332P/387R/390A/749F/750D/1218M/1248D/1377D/1439E, 63Q/171P/260T/306G/332P/387I/390A/750D/1051R/1148N/1193A/1218G/1248D/1377D/1439E, 171P/260T/387I/750D/924I/1248D/1377D/1439E, 171P/260T/387I/750D/755S/1248D/1377D/1439E, 171P/260T/387I/750D/752F/1248D/1377D/1439E, 171P/260T/387I/750D/S751Y/1248D/1377D/1439E, 171P/260T/387I/750D/923M/1248D/1377D/1439E, 171P/260T/387I/750D/797Y/1248D/1377D/1439E, 171P/260T/387I/750D/754W/1248D/1377D/1439E, 171P/260T/387I/750D/793G/1248D/1377D/1439E, 171P/260T/387I/750D/753D/1248D/1377D/1439E, 171P/260T/387I/750D/922W/1248D/1377D/1439E, 171P/260T/387I/750D/896L/1248D/1377D/1439E, 171P/260T/387I/750D/938R/1248D/1377D/1439E, 171P/260T/387I/750D/775G/1248D/1377D/1439E, 171P/260T/387I/750D/751E/1248D/1377D/1439E, 171P/260T/387I/750D/732W/1248D/1377D/1439E, 171P/260T/387I/750D/752G/1248D/1377D/1439E, 171P/260T/387I/750D/810V/1248D/1377D/1439E, 171P/260T/387I/750D/938V/1248D/1377D/1439E, 171P/260T/387I/750D/747D/1248D/1377D/1439E, 171P/260T/387I/750D/755G/1248D/1377D/1439E, 171P/260T/387I/393G/407V/750D/1248D/1377D/1439E, 171P/260T/387I/750D/797W/1248D/1377D/1439E, 171P/260T/387I/750D/819V/1248D/1377D/1439E, 171P/260T/387I/393G/750D/1248D/1377D/1439E, 171P/260T/387I/750D/787W/1248D/1377D/1439E, 171P/260T/387I/393R/750D/1248D/1377D/1439E, 171P/260T/387I/750D/802V/1248D/1377D/1439E, 171P/260T/387I/750D/775E/1248D/1377D/1439E, 171P/260T/387I/750D/924P/1248D/1377D/1439E, 171P/260T/387I/732G/750D/1248D/1377D/1439E, 171P/260T/387I/750D/811F/1248D/1377D/1439E, 171P/260T/387I/750D/802S/1248D/1377D/1439E, 171P/260T/387I/750D/752L/1248D/1377D/1439E, 171P/260T/387I/392A/750D/1248D/1377D/1439E, or 171P/260T/387I/397G/750D/1248D/1377D/1439E, wherein the amino acid positions are relative to the reference sequence corresponding to SEQ ID NO: 2.

In some embodiments, the amino acid sequence of the recombinant factor VIII polypeptide comprises at least a substitution or substitution set at amino acid position(s) 171/260/306/332/387/390/620/749/750/1248/1377/1439, 171/260/306/332/387/390/491/749/750/1248/1377/1439, 171/260/306/332/387/390/749/750/1229/1248/1377/1439, 171/260/306/332/387/390/615/749/750/1248/1377/1439, 171/260/306/332/387/390/749/750/1248/1283/1377/1439, 171/260/306/332/387/390/749/750/822/1248/1377/1439, 171/260/306/328/332/387/390/749/750/1248/1377/1439, 171/212/260/306/332/387/390/749/750/1248/1377/1439, 171/260/306/332/387/390/749/750/818/1248/1377/1439, 171/260/306/332/387/390/555/684/749/750/1248/1377/1439, 171/260/306/332/387/390/749/750/828/1248/1377/1439, 171/260/306/332/387/390/550/749/750/1248/1377/1439, 171/260/306/332/387/390/749/750/830/1248/1377/1439, 171/260/306/332/387/390/749/750/839/1248/1377/1439, 13/171/260/306/332/387/390/749/750/1248/1377/1439, 14/171/260/306/332/387/390/749/750/1248/1377/1439, 17/171/260/306/332/387/390/749/750/1248/1377/1439, 171/260/306/332/387/390/749/750/836/1248/1377/1439, 171/260/306/332/387/390/749/750/1210/1248/1377/1439, 171/260/306/332/387/390/749/750/1248/1281/1377/1439, 64/171/260/306/332/387/390/749/750/1248/1377/1439, 171/260/306/332/387/390/682/749/750/1248/1377/1439, 171/260/306/318/332/387/390/749/750/1248/1377/1439, 20/171/260/306/332/387/390/749/750/1248/1377/1439, 21/171/260/306/332/387/390/749/750/1248/1377/1439, 171/260/306/325/332/387/390/749/750/1248/1377/1439, 171/260/306/332/387/390/533/749/750/1248/1377/1439, 19/171/260/306/332/387/390/749/750/1248/1377/1439, 171/260/306/332/387/390/749/750/817/1248/1377/1439, 171/260/306/332/387/390/749/750/831/1248/1377/1439, 171/225/260/306/332/387/390/749/750/1248/1377/1439, 171/260/306/332/387/390/749/750/1156/1248/1377/1439, 171/260/306/332/387/390/393/747/749/750/1248/1377/1439, 171/260/306/332/387/390/749/750/753/1248/1377/1439, 171/260/306/332/387/390/393/732/749/750/751/1248/1377/1439, 63/171/260/306/332/387/390/749/750/797/1248/1377/1439, 171/260/306/332/387/390/622/749/750/1248/1377/1439, 171/260/306/332/387/390/617/749/750/1248/1377/1439, 171/260/306/332/387/390/749/750/907/1248/1377/1439, 171/260/306/332/387/390/749/750/1040/1248/1377/1439, 171/260/306/332/387/390/611/749/750/1248/1377/1439, 171/260/306/332/387/390/488/749/750/1248/1377/1439, 171/260/306/332/387/390/749/750/1192/1248/1377/1439, 171/260/306/332/387/390/749/750/1194/1248/1377/1439, 171/260/306/332/387/390/619/749/750/1248/1377/1439, 16/171/260/306/332/387/390/749/750/1248/1377/1439, 171/260/306/332/387/390/749/750/802/1248/1377/1439, 171/211/260/306/332/387/390/749/750/1248/1377/1439, 171/260/306/332/387/390/749/836/750/1248/1377/1439, 171/260/306/332/387/390/555/749/750/1248/1377/1439, 171/260/306/332/387/390/536/749/750/1248/1377/1439, 171/260/306/332/387/390/527/749/750/1248/1377/1439, 171/260/306/332/387/390/749/750/841/1248/1377/1439, 171/260/306/320/332/387/390/749/750/1248/1377/1439, 171/260/306/332/387/390/749/750/837/1248/1377/1439, 171/260/306/332/387/390/558/749/750/1248/1377/1439, 171/260/306/332/387/390/749/750/914/1248/1377/1439, 171/260/306/312/332/387/390/749/750/1248/1377/1439, 171/260/306/332/387/390/681/749/750/1248/1377/1439, 171/260/306/332/387/390/749/750/838/1248/1377/1439, 15/171/260/306/332/387/390/749/750/1248/1377/1439, 171/260/306/332/387/390/749/750/1248/1377/1427/1439, 18/171/260/306/332/387/390/749/750/1248/1377/1439, 171/260/306/332/387/390/683/749/750/1248/1377/1439, 24/171/260/306/332/387/390/749/750/1248/1377/1439, 171/260/306/332/387/390/534/749/750/1248/1377/1439, 171/260/306/332/387/390/749/750/840/1248/1377/1439, 171/260/306/332/387/390/749/750/1160/1248/1377/1439, 171/260/306/332/387/390/551/749/750/1248/1377/1439, 171/260/306/332/387/390/393/749/750/1248/1377/1439, 171/260/306/332/387/390/393/747/749/750/752/811/819/923/1148 1248/1377/1439, 171/260/306/332/387/390/393/749/750/751/775/1248/1377/1439, 63/171/260/306/332/387/390/393/732/749/750/775/1148/1248/1377/1439, 171/260/306/332/387/390/393/732/749/750/1148/1248/1377/1439, 171/260/306/332/387/390/749/750/775/1148/1248/1377/1439, 171/260/306/332/387/390/393/749/750/752/753/775/793/797/1248/1377/1439, 171/260/306/332/387/390/393/749/750/923/1248/1377/1439, 171/260/306/332/387/390/393/749/750/923/1148/1248/1377/1439, 171/260/306/332/387/390/393/747/749/750/751/1248/1377/1439, 171/260/306/332/387/390/393/747/749/750/753/775/1148/1248/1377/1439, 171/260/306/332/387/390/749/750/752/923/1051/1248/1377/1439, 63/171/260/306/332/387/390/749/750/775/1248/1377/1439, 63/171/260/306/332/387/390/393/749/750/752/775/793/1051/1053 1248/1377/1439, 171/260/306/332/387/390/749/750/752/753/775/793/819/1248/1377/1439, 63/171/260/306/332/387/390/749/750/1051/1248/1377/1439, 63/171/260/306/332/387/390/747/749/750/752/753/775/811/819/923/1051 1248/1377/1439, 171/260/306/332/387/390/393/749/750/752/787/1248/1377/1439, 171/260/306/332/387/390/749/750/751/1248/1377/1439, 63/171/260/306/332/387/390/749/750/1148/1248/1377/1439, 63/171/260/306/332/387/390/747/749/750/752/753/819/923/1148 1248/1377/1439, 171/260/306/332/387/390/749/750/1148/1248/1377/1439, 171/260/306/332/387/390/747/749/750/752/753/793/811/1248/1377/1439, 63/171/260/306/332/387/390/749/750/1248/1377/1439, 171/260/306/332/387/390/749/750/797/1148/1248/1377/1439, 171/260/306/332/387/390/749/750/751/752/753/1051/1248/1377/1439, 63/171/260/306/332/387/390/732/749/750/1051/1248/1377/1439, 171/260/306/332/387/390/749/750/752/797/815/923/1051/1148 1248/1377/1439, or 171/260/306/332/387/390/747/749/750/753/1248/1377/1439, wherein the amino acid positions are relative to the reference sequence corresponding to SEQ ID NO: 2.

In some embodiments, the amino acid sequence of the recombinant factor VIII polypeptide comprises at least a substitution or substitution set 171P/260T/306G/332P/387I/390A/620N/749F/750D/1248D/1377D/1439E, 171P/260T/306G/332P/387I/390A/491E/749F/750D/1248D/1377D/1439E, 171P/260T/306G/332P/387I/390A/749F/750D/491/1248D/1377D/1439E, 171P/260T/306G/332P/387I/390A/615N/749F/750D/1248D/1377D/1439E, 171P/260T/306G/332P/387I/390A/749F/750D/1248D/1283 S/1377D/1439E, 171P/260T/306G/332P/387I/390A/749F/750D/822S/1248D/1377D/1439E, 171P/260T/306G/328G/332P/387I/390A/749F/750D/1248D/1377D/1439E, 171P/212L/260T/306G/332P/387I/390A/749F/750D/1248D/1377D/1439E, 171P/260T/306G/332P/387I/390A/749F/750D/818W/1248D/1377D/1439E, 171P/260T/306G/332P/387I/390A/749F/750D/1248D/1283L/1377D/1439E, 171P/260T/306G/332P/387I/390A/555G/684G/749F/750D/1248D/1377D/1439E, 171P/260T/306G/332P/387I/390A/749F/750D/828K/1248D/1377D/1439E, 171P/260T/306G/332P/387I/390A/550G/749F/750D/1248D/1377D/1439E, 171P/260T/306G/332P/387I/390A/749F/750D/830M/1248D/1377D/1439E, 171P/260T/306G/332P/387I/390A/749F/750D/839A/1248D/1377D/1439E, 171P/260T/306G/332P/387I/390A/749F/750D/822T/1248D/1377D/1439E, 13Y/171P/260T/306G/332P/387I/390A/749F/750D/1248D/1377D/1439E, 14G/171P/260T/306G/332P/387I/390A/749F/750D/1248D/1377D/1439E, 17V/171P/260T/306G/332P/387I/390A/749F/750D/1248D/1377D/1439E, 171P/260T/306G/332P/387I/390A/749F/750D/836V/1248D/1377D/1439E, 171P/260T/306G/332P/387I/390A/749F/750D/839P/1248D/1377D/1439E, 171P/260T/306G/332P/387I/390A/749F/750D/1210W/1248D/1377D/1439E, 171P/260T/306G/332P/387I/390A/749F/750D/1248D/1281R/1377D/1439E, 64D/171P/260T/306G/332P/387I/390A/749F/750D/1248D/1377D/1439E, 171P/212G/260T/306G/332P/387I/390A/749F/750D/1248D/1377D/1439E, 171P/260T/306G/332P/387I/390A/682R/749F/750D/1248D/1377D/1439E, 14M/171P/260T/306G/332P/387I/390A/749F/750D/1248D/1377D/1439E, 171P/260T/306G/318C/332P/387I/390A/749F/750D/1248D/1377D/1439E, 17S/171P/260T/306G/332P/387I/390A/749F/750D/1248D/1377D/1439E, 20L/171P/260T/306G/332P/387I/390A/749F/750D/1248D/1377D/1439E, 171P/260T/306G/332P/387I/390A/749F/750D/818G/1248D/1377D/1439E, 171P/260T/306G/332P/387I/390A/749F/750D/836G/1248D/1377D/1439E, 21M/171P/260T/306G/332P/387I/390A/749F/750D/1248D/1377D/1439E, 171P/260T/306G/332P/387I/390A/749F/750D/836R/1248D/1377D/1439E, 171P/260T/306G/325Y/332P/387I/390A/749F/750D/1248D/1377D/1439E, 171P/260T/306G/332P/387I/390A/533V/749F/750D/1248D/1377D/1439E, 19G/171P/260T/306G/332P/387I/390A/749F/750D/1248D/1377D/1439E, 14V/171P/260T/306G/332P/387I/390A/749F/750D/1248D/1377D/1439E, 171P/260T/306G/332P/387I/390A/749F/750D/817D/1248D/1377D/1439E, 171P/260T/306G/332P/387I/390A/749F/750D/831W/1248D/1377D/1439E, 171P/225R/260T/306G/332P/387I/390A/749F/750D/1248D/1377D/1439E, 171P/260T/306G/332P/387I/390A/749F/750D/1156R/1248D/1377D/1439E, 171P/260T/306G/332P/387I/390A/749F/750D/830V/1248D/1377D/1439E, 171P/260T/306G/332P/387I/390A/393G/A747D/749F/750D/1248D/1377D/1439E, 171P/260T/306G/332P/387I/390A/749F/750D/753D/1248D/1377D/1439E, 171P/260T/306G/332P/387I/390A/393G/732W/749F/750D/S751Y/1248D/1377D/1439E, 63Q/171P/260T/306G/332P/387I/390A/749F/750D/797W/1248D/1377D/1439E, 171P/260T/306G/332P/387I/390A/622D/749F/750D/1248D/1377D/1439E, 171P/260T/306G/332P/387I/390A/617E/749F/750D/1248D/1377D/1439E, 171P/260T/306G/332P/387I/390A/617N/749F/750D/1248D/1377D/1439E, 171P/260T/306G/332P/387I/390A/749F/750D/907D/1248D/1377D/1439E, 171P/260T/306G/332P/387I/390A/749F/750D/1040E/1248D/1377D/1439E, 171P/260T/306G/332P/387I/390A/617D/749F/750D/1248D/1377D/1439E, 171P/260T/306G/332P/387I/390A/611E/749F/750D/1248D/1377D/1439E, 171P/260T/306G/332P/387I/390A/620P/749F/750D/1248D/1377D/1439E, 171P/260T/306G/332P/387I/390A/617G/749F/750D/1248D/1377D/1439E, 171P/260T/306G/332P/387I/390A/488G/749F/750D/1248D/1377D/1439E, 171P/260T/306G/332P/387I/390A/749F/750D/1192D/1248D/1377D/1439E, 171P/260T/306G/332P/387I/390A/749F/750D/1194P/1248D/1377D/1439E, 171P/260T/306G/332P/387I/390A/619E/749F/750D/1248D/1377D/1439E, 171P/260T/306G/332P/387I/390A/615D/749F/750D/1248D/1377D/1439E, 16A/171P/260T/306G/332P/387I/390A/749F/750D/1248D/1377D/1439E, 171P/260T/306G/332P/387I/390A/749F/750D/802S/1248D/1377D/1439E, 171P/260T/306G/332P/387I/390A/749F/750D/818V/1248D/1377D/1439E, 171P/L211K/260T/306G/332P/387I/390A/749F/750D/1248D/1377D/1439E, 171P/260T/306G/332P/387I/390A/749F/836W/750D/1248D/1377D/1439E, 171P/260T/306G/332P/387I/390A/555R/749F/750D/1248D/1377D/1439E, 171P/260T/306G/332P/387I/390A/536A/749F/750D/1248D/1377D/1439E, 171P/260T/306G/332P/387I/390A/527A/749F/750D/1248D/1377D/1439E, 171P/260T/306G/332P/387I/390A/555E/749F/750D/1248D/1377D/1439E, 171P/260T/306G/332P/387I/390A/749F/750D/841W/1248D/1377D/1439E, 171P/260T/306G/320Q/332P/387I/390A/749F/750D/1248D/1377D/1439E, 171P/260T/306G/332P/387I/390A/749F/750D/837G/1248D/1377D/1439E, 171P/260T/306G/332P/387I/390A/558R/749F/750D/1248D/1377D/1439E, 171P/260T/306G/332P/387I/390A/555G/749F/750D/1248D/1377D/1439E, 171P/211R/260T/306G/332P/387I/390A/749F/750D/1248D/1377D/1439E, 64G/171P/260T/306G/332P/387I/390A/749F/750D/1248D/1377D/1439E, 171P/260T/306G/320R/332P/387I/390A/749F/750D/1248D/1377D/1439E, 171P/260T/306G/332P/387I/390A/533Y/749F/750D/1248D/1377D/1439E, 171P/260T/306G/332P/387I/390A/749F/750D/914V/1248D/1377D/1439E, 16V/171P/260T/306G/332P/387I/390A/749F/750D/1248D/1377D/1439E, 171P/260T/306G/332P/387I/390A/749F/750D/1248D/1283E/1377D/1439E, 171P/260T/306G/332P/387I/390A/536M/749F/750D/1248D/1377D/1439E, 171P/260T/306G/312L/332P/387I/390A/749F/750D/1248D/1377D/1439E, 171P/260T/306G/332P/387I/390A/681N/749F/750D/1248D/1377D/1439E, 171P/260T/306G/332P/387I/390A/749F/750D/838T/1248D/1377D/1439E, 171P/260T/306G/332P/387I/390A/536R/749F/750D/1248D/1377D/1439E, 15G/171P/260T/306G/332P/387I/390A/749F/750D/1248D/1377D/1439E, 171P/260T/306G/332P/387I/390A/536P/749F/750D/1248D/1377D/1439E, 171P/260T/306G/332P/387I/390A/558W/749F/750D/1248D/1377D/1439E, 171P/260T/306G/332P/387I/390A/558E/749F/750D/1248D/1377D/1439E, 171P/260T/306G/332P/387I/390A/749F/750D/1248D/1377D/1427Y/1439E, 171P/260T/306G/332P/387I/390A/749F/750D/839G/1248D/1377D/1439E, 18L/171P/260T/306G/332P/387I/390A/749F/750D/1248D/1377D/1439E, 13G/171P/260T/306G/332P/387I/390A/749F/750D/1248D/1377D/1439E, 171P/260T/306G/332P/387I/390A/749F/750D/1210M/1248D/1377D/1439E, 171P/260T/306G/332P/387I/390A/749F/750D/1156G/1248D/1377D/1439E, 171P/260T/306G/332P/387I/390A/558L/749F/750D/1248D/1377D/1439E, 171P/260T/306G/332P/387I/390A/749F/750D/839K/1248D/1377D/1439E, 171P/211P/260T/306G/332P/387I/390A/749F/750D/1248D/1377D/1439E, 171P/211G/260T/306G/332P/387I/390A/749F/750D/1248D/1377D/1439E, 171P/211Q/260T/306G/332P/387I/390A/749F/750D/1248D/1377D/1439E, 16D/171P/260T/306G/332P/387I/390A/749F/750D/1248D/1377D/1439E, 171P/260T/306G/M320G/332P/387I/390A/749F/750D/1248D/1377D/1439E, 171P/260T/306G/332P/387I/390A/683W/749F/750D/1248D/1377D/1439E, 24V/171P/260T/306G/332P/387I/390A/749F/750D/1248D/1377D/1439E, 171P/260T/306G/332P/387I/390A/534I/749F/750D/1248D/1377D/1439E, 171P/260T/306G/332P/387I/390A/681A/749F/750D/1248D/1377D/1439E, 171P/260T/306G/332P/387I/390A/749F/750D/840G/1248D/1377D/1439E, 171P/260T/306G/332P/387I/390A/749F/750D/1160R/1248D/1377D/1439E, 171P/260T/306G/332P/387I/390A/681C/749F/750D/1248D/1377D/1439E, 171P/260T/306G/328Q/332P/387I/390A/749F/750D/1248D/1377D/1439E, 15S/171P/260T/306G/332P/387I/390A/749F/750D/1248D/1377D/1439E, 171P/260T/306G/332P/387I/390A/536K/749F/750D/1248D/1377D/1439E, 171P/260T/306G/332P/387I/390A/536G/749F/750D/1248D/1377D/1439E, 171P/260T/306G/332P/387I/390A/533R/749F/750D/1248D/1377D/1439E, 171P/260T/306G/332P/387I/390A/681E/749F/750D/1248D/1377D/1439E, 171P/260T/306G/332P/387I/390A/536W/749F/750D/1248D/1377D/1439E, 171P/260T/306G/332P/387I/390A/749F/750D/1156N/1248D/1377D/1439E, 16P/171P/260T/306G/332P/387I/390A/749F/750D/1248D/1377D/1439E, 171P/260T/306G/332P/387I/390A/749F/750D/817A/1248D/1377D/1439E, 18R/171P/260T/306G/332P/387I/390A/749F/750D/1248D/1377D/1439E, 171P/260T/306G/332P/387I/390A/551W/749F/750D/1248D/1377D/1439E, 171P/260T/306G/332P/387I/390A/555T/749F/750D/1248D/1377D/1439E, 171P/260T/306G/332P/387I/390A/681G/749F/750D/1248D/1377D/1439E, 171P/212A/260T/306G/332P/387I/390A/749F/750D/1248D/1377D/1439E, 16G/171P/260T/306G/332P/387I/390A/749F/750D/1248D/1377D/1439E, 171P/260T/306G/332P/387I/390A/749F/750D/838G/1248D/1377D/1439E, 171P/260T/306G/332P/387I/390A/681R/749F/750D/1248D/1377D/1439E, 171P/260T/306G/332P/387I/390A/393G/749F/750D/1248D/1377D/1439E, 171P/260T/306G/332P/387I/390A/393G/747D/749F/750D/752G/811F/819V/923M/1148N/1248D/1377D/1439E, 171P/260T/306G/332P/387I/390A/393G/749F/750D/751Y/775G/1248D/1377D/1439E, 63Q/171P/260T/306G/332P/387I/390A/393G/732W/749F/750D/775G/1148N/1248D/1377D/1439E, 171P/260T/306G/332P/387I/390A/393G/732W/749F/750D/1148N/1248D/1377D/1439E, 171P/260T/306G/332P/387I/390A/749F/750D/775G/1148N/1248D/1377D/1439E, 171P/260T/306G/332P/387I/390A/393G/749F/750D/752L/753D/775G/793G/797W/1248D/1377D/1439E, 171P/260T/306G/332P/387I/390A/393G/749F/750D/923M/1248D/1377D/1439E, 171P/260T/306G/332P/387I/390A/393G/749F/750D/923M/1148N/1248D/1377D/1439E, 171P/260T/306G/332P/387I/390A/393G/747D/749F/750D/751Y/1248D/1377D/1439E, 171P/260T/306G/332P/387I/390A/393G/747D/749F/750D/753D/775G/1148N/1248D/1377D/1439E, 171P/260T/306G/332P/387I/390A/749F/750D/752L/923M/1051R/1248D/1377D/1439E, 63Q/171P/260T/306G/332P/387I/390A/749F/750D/775G/1248D/1377D/1439E, 63Q/171P/260T/306G/332P/387I/390A/393G/749F/750D/752L/775G/793G/1051R/1053R 1248D/1377D/1439E, 171P/260T/306G/332P/387I/390A/749F/750D/752G/753D/775G/793G/819V/1248D/1377D/1439E, 63Q/171P/260T/306G/332P/387I/390A/749F/750D/1051R/1248D/1377D/1439E, 63Q/171P/260T/306G/332P/387I/390A/747D/749F/750D/752G/753D/775G/811F/819V/923M/1051R 1248D/1377D/1439E, 171P/260T/306G/332P/387I/390A/393G/749F/750D/752G/787W/1248D/1377D/1439E, 171P/260T/306G/332P/387I/390A/749F/750D/751Y/1248D/1377D/1439E, 63Q/171P/260T/306G/332P/387I/390A/749F/750D/1148N/1248D/1377D/1439E, 63H1/171P/260T/306G/332P/387I/390A/747D/749F/750D/752G/753D/819V/923M/1148N/1248D/1377D/1439E, 171P/260T/306G/332P/387I/390A/749F/750D/1148N/1248D/1377D/1439E, 171P/260T/306G/332P/387I/390A/747D/749F/750D/752L/753D/793G/811F/1248D/1377D/1439E, 63Q/171P/260T/306G/332P/387I/390A/749F/750D/1248D/1377D/1439E, 171P/260T/306G/332P/387I/390A/749F/750D/797W/1148N/1248D/1377D/1439E, 171P/260T/306G/332P/387I/390A/749F/750D/751Y/752L/753D/1051R/1248D/1377D/1439E, 63Q/171P/260T/306G/332P/387I/390A/732W/749F/750D/1051R/1248D/1377D/1439E, 171P/260T/306G/332P/387I/390A/749F/750D/752G/797W/815I/923M/1051R/1148N 1248D/1377D/1439E, or 171P/260T/306G/332P/387I/390A/747D/749F/750D/753D/1248D/1377D/1439E, wherein the amino acid positions are relative to the reference sequence corresponding to SEQ ID NO: 2.

In some embodiments, the amino acid sequence of the recombinant factor VIII polypeptide comprises at least a substitution or substitution set at amino acid position(s) 63/171/260/306/332/387/390/393/681/732/749/750/775/1148/1248/1377/1439, 63/171/260/306/332/387/390/393/622/732/749/750/775/1148/1248/1377/1439, 63/171/260/306/332/387/390/393/617/732/749/750/775/1148/1248/1377/1439, 63/171/260/306/332/387/390/393/558/732/749/750/775/1148/1248/1377/1439, 63/171/260/306/332/387/390/393/536/732/749/750/775/1148/1248/1377/1439, 63/171/211/260/306/332/387/390/393/732/749/750/775/1148/1248/1377/1439, 63/171/260/306/320/332/387/390/393/732/749/750/775/1148/1248/1377/1439, 63/171/260/306/332/387/390/393/732/749/750/775/839/1148/1248/1377/1439, 63/171/260/306/332/387/390/393/732/749/750/775/797/1148/1248/1377/1439, 63/171/260/306/332/387/390/393/533/732/749/750/775/1148/1248/1377/1439, 63/171/260/306/332/387/390/393/732/749/750/751/775/1148/1248/1377/1439, 63/171/260/303/306/332/387/390/393/732/749/750/775/1148/1248/1377/1439, 63/171/260/306/332/387/390/393/732/749/750/951/775/1148/1248/1377/1439, 63/164/171/260/306/332/387/390/393/732/749/750/775/1148/1248/1377/1439, 63/171/260/306/332/387/390/393/732/749/750/775/818/1148/1248/1377/1439, 63/171/260/306/332/387/390/393/732/749/750/775/807/1148/1248/1377/1439, 63/171/260/306/332/387/390/393/732/749/750/775/799/1148/1248/1377/1439, 63/171/260/306/332/387/390/393/732/735/749/750/775/1148/1248/1377/1439, 63/171/260/306/332/387/390/393/732/749/750/775/794/1148/1248/1377/1439, 63/171/260/306/332/387/390/393/732/749/750/775/926/1148/1248/1377/1439, 63/171/260/306/332/387/390/393/732/749/750/755/775/1148/1248/1377/1439, 63/171/260/306/332/387/390/393/732/749/750/775/921/1148/1248/1377/1439, 63/171/260/306/332/387/390/393/732/749/750/775/801/1148/1248/1377/1439, 63/171/260/306/332/387/390/393/732/749/750/775/1094/1148/1248/1377/1439, 63/171/260/306/332/387/390/393/732/749/750/775/968/1148/1248/1377/1439, 63/171/260/306/332/387/390/393/732/749/750/775/929/1148/1248/1377/1439, 63/171/260/306/332/387/390/393/732/749/750/775/924/1148/1248/1377/1439, 63/171/260/306/332/387/390/393/698/732/749/750/775/1148/1248/1377/1439, 63/168/171/260/306/332/387/390/393/732/749/750/775/1148/1248/1377/1439, 63/171/260/306/332/387/390/393/581/732/749/750/775/1148/1248/1377/1439, 63/171/260/306/332/387/390/393/732/749/750/775/916/1148/1248/1377/1439, 63/171/260/306/332/387/390/393/732/749/750/775/1148/1248/1377/1428/1439, 63/171/260/306/332/387/390/393/732/749/750/775/805/1148/1248/1377/1439, 63/171/260/306/332/387/390/393/732/749/750/775/1114/1148/1248/1377/1439, 63/171/260/306/311/332/387/390/393/732/749/750/775/1148/1248/1377/1439, 63/171/260/306/332/387/390/393/732/737/749/750/775/1148/1248/1377/1439, 63/171/260/306/332/387/390/393/693/732/749/750/775/1148/1248/1377/1439, 63/171/260/306/332/387/390/393/732/749/750/775/809/1148/1248/1377/1439, 63/171/260/306/332/387/390/393/732/749/750/757/775/1148/1248/1377/1439, 63/171/260/306/332/387/390/393/732/749/750/775/1099/1148/1248/1377/1439, 63/171/260/306/332/387/390/393/676/732/749/750/775/1148/1248/1377/1439, 63/171/260/306/332/387/390/393/732/740/749/750/775/1148/1248/1377/1439, 63/171/260/306/332/387/390/393/732/698/749/750/775/1148/1248/1377/1439, 63/171/260/306/332/387/390/393/732/749/750/775/791/1148/1248/1377/1439, 63/171/211/260/306/332/387/390/393/536/622/681/732/749/750/775/1148/1248/1377/1439, 63/171/260/306/332/387/390/393/536/558/732/749/750/775/1148/1248/1377/1439, 63/171/211/260/306/332/387/390/393/536/555/617/749/750/775/1148/1248/1377/1439, 63/171/211/260/306/332/387/390/393/681/749/750/775/1148/1248/1377/1439, 63/171/211/260/306/332/387/390/393/536/555/732/749/750/775/1148/1248/1377/1439, 63/171/260/306/332/387/390/393/555/732/749/750/775/1148/1248/1377/1439, 63/171/211/260/306/332/387/390/393/536/732/749/750/775/1148/1248/1377/1439, 63/171/211/212/260/306/332/387/390/393/732/749/750/775/1148/1248/1377/1439, 63/171/211/260/306/332/387/390/393/536/617/681/749/750/775/1148/1248/1377/1439, 63/171/211/260/306/332/387/390/393/536/558/617/681/732/749/750/775/1148/1248/1377/1439, 63/171/260/306/332/387/390/393/555/749/750/775/1148/1248/1377/1439, 63/171/260/306/332/387/390/393/536/555/558/617/749/750/775/1148/1248/1377/1439, 63/171/260/306/332/387/390/393/536/617/681/749/750/775/1148/1248/1377/1439, 63/171/260/306/332/387/390/393/536/555/732/749/750/775/1148/1248/1377/1439, 63/171/260/306/332/387/390/393/536/622/681/732/749/750/775/1148/1248/1377/1439, 63/171/260/306/332/387/390/393/749/750/775/1148/1248/1377/1439, 63/171/212/260/306/320/332/387/390/393/533/617/681/732/749/750/775/1148/1248/1377/1439, 63/171/211/260/306/312/332/387/390/393/533/534/617/619/681/732/749/750/775/839/1148/1248/1377/1439, 63/171/212/260/306/332/387/390/393/732/749/750/775/1148/1248/1377/1439, 63/171/211/212/260/306/320/332/387/390/393/619/681/732/749/750/775/839/1148/1248/1377/1439, 63/171/260/306/332/387/390/393/732/749/750/775/797/839/1148/1248/1377/1439, 63/171/212/260/306/312/320/332/387/390/393/533/534/617/681/732/749/750/775/1148/1248/1377/1439, 63/171/260/306/332/387/390/393/558/619/681/732/749/750/775/1148/1248/1377/1439, 63/171/260/306/320/332/387/390/393/558/681/732/749/750/775/1148/1248/1377/1439, 63/171/212/260/306/320/332/387/390/393/534/619/681/732/749/750/775/797/839/1148/1248/1377/1439, 63/171/260/306/320/332/387/390/393/558/619/749/750/775/839/1148/1248/1377/1439, 63/171/211/212/260/306/312/332/387/390/393/534/558/619/681/749/750/775/839/1148/1248/1377/1439, 63/171/260/306/320/332/387/390/393/534/558/619/749/750/775/797/1148/1248/1377/1439, 63/171/260/306/332/387/390/393/558/617/619/681/732/749/750/775/1148/1248/1377/1439, 63/171/211/260/306/332/387/390/393/534/558/611/681/732/749/750/775/1148/1248/1377/1439, 63/171/211/260/306/320/332/387/390/393/617/619/681/732/749/750/775/1148/1248/1377/1439, 63/171/260/306/332/387/390/393/558/617/681/732/749/750/775/797/1148/1248/1377/1439, 63/171/260/306/320/332/387/390/393/534/619/749/750/775/1148/1248/1377/1439, 63/171/211/260/306/332/387/390/393/619/681/732/749/750/775/1148/1248/1377/1439, 63/171/211/212/260/306/332/387/390/393/534/558/619/749/750/775/839/1148/1248/1377/1439, 63/171/212/260/306/320/332/387/390/393/619/681/732/749/750/775/797/839/1148/1248/1377/1439, 63/171/212/260/306/320/332/387/390/393/611/732/749/750/775/839/1148/1248/1377/1439, 63/171/260/306/320/332/387/390/393/558/619/681/732/749/750/775/1148/1248/1377/1439, 63/171/260/306/312/332/387/390/393/533/617/681/732/749/750/775/797/839/1148/1248/1377/1439, 63/171/260/306/332/387/390/393/558/611/681/732/749/750/775/1148/1248/1377/1439, 63/171/260/306/312/332/387/390/393/732/749/750/775/1148/1248/1377/1439, 63/171/211/260/306/320/332/387/390/393/558/619/681/749/750/775/797/839/1148/1248/1377/1439, 63/171/260/306/320/332/387/390/393/558/749/750/775/797/1148/1248/1377/1439, 63/171/260/306/332/387/390/393/533/558/611/619/716/749/750/775/839/1148/1248/1377/1439, 63/171/260/306/312/332/387/390/393/681/732/749/750/775/1148/1248/1377/1439, 63/171/260/306/320/332/387/390/393/534/619/681/732/749/750/775/1148/1248/1377/1439, 63/171/211/260/306/320/332/387/390/393/533/536/619/732/749/750/775/1148/1248/1377/1439, 63/171/260/306/332/387/390/393/558/617/681/732/749/750/775/839/1148/1248/1377/1439, 63/171/212/260/306/320/332/387/390/393/534/558/681/732/749/750/775/1148/1248/1377/1439, 63/171/260/306/332/387/390/393/533/534/681/732/749/750/775/839/1148/1248/1377/1439, 63/171/212/260/306/332/387/390/393/534/619/681/732/749/750/775/1148/1248/1377/1439, 63/171/260/306/332/387/390/393/617/619/681/732/749/750/775/1148/1248/1377/1439, 63/171/211/212/260/306/320/332/387/390/393/558/617/681/732/749/750/775/1148/1248/1377/1439, 63/171/212/260/306/332/387/390/393/534/558/681/732/749/750/775/797/1148/1248/1377/1439, 63/171/212/260/306/320/332/387/390/393/534/732/749/750/775/1148/1248/1377/1439, 63/171/211/260/306/312/332/387/390/393/619/732/749/750/775/1148/1248/1377/1439, 63/171/212/260/306/312/332/387/390/393/619/732/749/750/775/839/1148/1248/1377/1439, 63/171/210/212/260/306/320/332/387/390/393/558/681/732/749/750/775/1148/1248/1377/1439, 63/171/212/260/306/312/332/387/390/393/534/681/732/749/750/775/797/839/1148/1248/1377/1439, 63/171/211/260/306/320/332/387/390/393/558/732/749/750/775/839/1148/1248/1377/1439, 63/171/260/294/306/332/387/390/393/732/749/750/775/1148/1248/1377/1439, 63/171/260/306/332/387/390/393/732/749/750/759/775/1148/1248/1377/1439, 63/171/260/306/332/387/390/393/538/732/749/750/775/1148/1248/1377/1439, or 63/171/260/306/332/387/390/393/732/749/750/775/1109/1148/1248/1377/1439, wherein the amino acid positions are relative to the reference sequence corresponding to SEQ ID NO: 2.

In some embodiments, the amino acid sequence of the recombinant factor VIII polypeptide comprises at least a substitution or substitution set 63Q/171P/260T/306G/332P/387I/390A/393G/681A/732W/749F/750D/775G/1148N/1248D/1377D/1439E, 63Q/171P/260T/306G/332P/387I/390A/393G/622D/732W/749F/750D/775G/1148N/1248D/1377D/1439E, 63Q/171P/260T/306G/332P/387I/390A/393G/617G/732W/749F/750D/775G/1148N/1248D/1377D/1439E, 63Q/171P/260T/306G/332P/387I/390A/393G/558L/732W/749F/750D/775G/1148N/1248D/1377D/1439E, 63Q/171P/260T/306G/332P/387I/390A/393G/536K/732W/749F/750D/775G/1148N/1248D/1377D/1439E, 63Q/171P/260T/306G/332P/387I/390A/393G/681G/732W/749F/750D/775G/1148N/1248D/1377D/1439E, 63Q/171P/211Q/260T/306G/332P/387I/390A/393G/732W/749F/750D/775G/1148N/1248D/1377D/1439E, 63Q/171P/260T/306G/320R/332P/387I/390A/393G/732W/749F/750D/775G/1148N/1248D/1377D/1439E, 63Q/171P/260T/306G/332P/387I/390A/393G/558W/732W/749F/750D/775G/1148N/1248D/1377D/1439E, 63Q/171P/260T/306G/332P/387I/390A/393G/732W/749F/750D/775G/839K/1148N/1248D/1377D/1439E, 63Q/171P/211K/260T/306G/332P/387I/390A/393G/732W/749F/750D/775G/1148N/1248D/1377D/1439E, 63Q/171P/260T/306G/332P/387I/390A/393G/732W/749F/750D/775G/797W/1148N/1248D/1377D/1439E, 63Q/171P/260T/306G/332P/387I/390A/393G/533R/732W/749F/750D/775G/1148N/1248D/1377D/1439E, 63Q/171P/260T/306G/332P/387I/390A/393G/732W/749F/750D/751A/775G/1148N/1248D/1377D/1439E, 63Q/171P/260T/306G/332P/387I/390A/393G/732W/749F/750D/751T/775G/1148N/1248D/1377D/1439E, 63Q/171P/260T/306G/332P/387I/390A/393G/732W/749F/750D/951V/775G/1148N/1248D/1377D/1439E, 63Q/164E/171P/260T/306G/332P/387I/390A/393G/732W/749F/750D/775G/1148N/1248D/1377D/1439E, 63Q/171P/260T/306G/332P/387I/390A/393G/732W/749F/750D/775G/818G/1148N/1248D/1377D/1439E, 63Q/171P/260T/306G/332P/387I/390A/393G/732W/749F/750D/775G/807L/1148N/1248D/1377D/1439E, 63Q/171P/260T/306G/332P/387I/390A/393G/732W/749F/750D/775G/799A/1148N/1248D/1377D/1439E, 63Q/171P/260T/306G/332P/387I/390A/393G/732W/735S/749F/750D/775G/1148N/1248D/1377D/1439E, 63Q/171P/260T/306G/332P/387I/390A/393G/732W/749F/750D/775G/794R/1148N/1248D/1377D/1439E, 63Q/171P/260T/306G/332P/387I/390A/393G/732W/749F/750D/775G/926V/1148N/1248D/1377D/1439E, 63Q/171P/260T/306G/332P/387I/390A/393G/732W/749F/750D/755P/775G/1148N/1248D/1377D/1439E, 63Q/171P/260T/306G/332P/387I/390A/393G/732W/749F/750D/775G/921W/1148N/1248D/1377D/1439E, 63Q/171P/260T/306G/332P/387I/390A/393G/732W/749F/750D/775G/801A/1148N/1248D/1377D/1439E, 63Q/171P/260T/306G/332P/387I/390A/393G/732W/749F/750D/775G/1094S/1148N/1248D/1377D/1439E, 63Q/171P/260T/306G/332P/387I/390A/393G/732W/749F/750D/775G/968A/1148N/1248D/1377D/1439E, 63Q/171P/260T/306G/332P/387I/390A/393G/732W/749F/750D/775G/929Q/1148N/1248D/1377D/1439E, 63Q/171P/260T/306G/332P/387I/390A/393G/732W/749F/750D/775G/924W/1148N/1248D/1377D/1439E, 63Q/171P/260T/306G/332P/387I/390A/393G/732W/749F/750D/775G/807P/1148N/1248D/1377D/1439E, 63Q/171P/260T/306G/332P/387I/390A/393G/698D/732W/749F/750D/775G/1148N/1248D/1377D/1439E, 63Q/168G/171P/260T/306G/332P/387I/390A/393G/732W/749F/750D/775G/1148N/1248D/1377D/1439E, 63Q/171P/260T/306G/332P/387I/390A/393G/732W/749F/750D/775G/929V/1148N/1248D/1377D/1439E, 63Q/171P/260T/306G/332P/387I/390A/393G/732W/749F/750D/775G/818A/1148N/1248D/1377D/1439E, 63Q/171P/260T/306G/332P/387I/390A/393G/732W/749F/750D/775G/818S/1148N/1248D/1377D/1439E, 63Q/171P/260T/306G/332P/387I/390A/393G/581K/732W/749F/750D/775G/1148N/1248D/1377D/1439E, 63Q/171P/260T/306G/332P/387I/390A/393G/732W/749F/750D/775G/818P/1148N/1248D/1377D/1439E, 63Q/171P/260T/306G/332P/387I/390A/393G/732W/749F/750D/775G/916K/1148N/1248D/1377D/1439E, 63Q/171P/260T/306G/332P/387I/390A/393G/698S/732W/749F/750D/775G/1148N/1248D/1377D/1439E, 63Q/171P/260T/751G/306G/332P/387I/390A/393G/732W/749F/750D/775G/1148N/1248D/1377D/1439E, 63Q/171P/260T/306G/332P/387I/390A/393G/732W/749F/750D/775G/916C/1148N/1248D/1377D/1439E, 63Q/171P/260T/306G/332P/387I/390A/393G/732W/749F/750D/775G/1148N/1248D/1377D/1428G/1439E, 63Q/171P/260T/306G/332P/387I/390A/393G/732W/749F/750D/775G/805L/1148N/1248D/1377D/1439E, 63Q/171P/260T/306G/332P/387I/390A/393G/732W/749F/750D/775G/924M/1148N/1248D/1377D/1439E, 63Q/171P/260T/306G/332P/387I/390A/393G/732W/749F/750D/775G/1114M/1148N/1248D/1377D/1439E, 63Q/171P/260T/306G/311G/332P/387I/390A/393G/732W/749F/750D/775G/1148N/1248D/1377D/1439E, 63Q/171P/260T/306G/332P/387I/390A/393G/732W/737N/749F/750D/775G/1148N/1248D/1377D/1439E, 63Q/171P/260T/306G/332P/387I/390A/393G/732W/749F/750D/775G/805V/1148N/1248D/1377D/1439E, 63Q/171P/260T/306G/332P/387I/390A/393G/732W/749F/750D/775G/801I/1148N/1248D/1377D/1439E, 63Q/171P/260T/306G/332P/387I/390A/393G/732W/749F/750D/751D/775G/1148N/1248D/1377D/1439E, 63Q/171P/260T/306G/332P/387I/390A/393G/693T/732W/749F/750D/775G/1148N/1248D/1377D/1439E, 63Q/171P/260T/306G/332P/387I/390A/393G/732W/749F/750D/775G/818E/1148N/1248D/1377D/1439E, 63Q/171P/260T/306G/332P/387I/390A/393G/732W/749F/750D/775G/929A/1148N/1248D/1377D/1439E, 63Q/171P/260T/306G/311E/332P/387I/390A/393G/732W/749F/750D/775G/1148N/1248D/1377D/1439E, 63Q/171P/260T/306G/332P/387I/390A/393G/732W/737L/749F/750D/775G/1148N/1248D/1377D/1439E, 63Q/171P/260T/306G/332P/387I/390A/393G/732W/749F/750D/775G/809W/1148N/1248D/1377D/1439E, 63Q/171P/260T/306G/332P/387I/390A/393G/732W/749F/750D/757A/775G/1148N/1248D/1377D/1439E, 63Q/171P/260T/306G/332P/387I/390A/393G/732W/749F/750D/775G/1099G/1148N/1248D/1377D/1439E, 63Q/171P/260T/306G/332P/387I/390A/393G/732W/749F/750D/775G/799G/1148N/1248D/1377D/1439E, 63Q/171P/260T/306G/332P/387I/390A/393G/732W/749F/750D/775G/916I/1148N/1248D/1377D/1439E, 63Q/171P/260T/306G/332P/387I/390A/393G/732W/749F/750D/775G/805G/1148N/1248D/1377D/1439E, 63Q/171P/260T/306G/332P/387I/390A/393G/732W/737F/749F/750D/775G/1148N/1248D/1377D/1439E, 63Q/171P/260T/306G/332P/387I/390A/393G/732W/737M/749F/750D/775G/1148N/1248D/1377D/1439E, 63Q/171P/260T/306G/332P/387I/390A/393G/732W/749F/750D/775G/799P/1148N/1248D/1377D/1439E, 63Q/171P/260T/306G/332P/387I/390A/393G/732W/749F/750D/775G/807C/1148N/1248D/1377D/1439E, 63Q/171P/260T/306G/332P/387I/390A/393G/732W/749F/750D/775G/916E/1148N/1248D/1377D/1439E, 63Q/171P/260T/306G/332P/387I/390A/393G/676Q/732W/749F/750D/775G/1148N/1248D/1377D/1439E, 63Q/171P/260T/306G/332P/387I/390A/393G/581V/732W/749F/750D/775G/1148N/1248D/1377D/1439E, 63Q/171P/260T/306G/332P/387I/390A/393G/732W/749F/750D/775G/968P/1148N/1248D/1377D/1439E, 63Q/171P/260T/306G/332P/387I/390A/393G/732W/749F/750D/755Y/775G/1148N/1248D/1377D/1439E, 63Q/171P/260T/306G/332P/387I/390A/393G/732W/749F/750D/775G/916Q/1148N/1248D/1377D/1439E, 63Q/171P/260T/306G/332P/387I/390A/393G/732W/735D/749F/750D/775G/1148N/1248D/1377D/1439E, 63Q/171P/260T/306G/332P/387I/390A/393G/732W/740G/749F/750D/775G/1148N/1248D/1377D/1439E, 63Q/171P/260T/306G/332P/387I/390A/393G/732W/698Y/749F/750D/775G/1148N/1248D/1377D/1439E, 63Q/171P/260T/306G/332P/387I/390A/393G/732W/749F/750D/757G/775G/1148N/1248D/1377D/1439E, 63Q/171P/260T/306G/332P/387I/390A/393G/732W/749F/750D/775G/791T/1148N/1248D/1377D/1439E, 63Q/171P/260T/306G/332P/387I/390A/393G/732W/749F/750D/775G/1114A/1148N/1248D/1377D/1439E, 63Q/171P/260T/306G/332P/387I/390A/393G/732W/735V/749F/750D/775G/1148N/1248D/1377D/1439E, 63Q/171P/260T/306G/332P/387I/390A/393G/732W/749F/750D/775G/1148N/1248D/1377D/1439E, 63Q/171P/260T/306G/332P/387I/390A/393G/536P/732W/749F/750D/775G/1148N/1248D/1377D/1439E, 63Q/171P/211R/260T/306G/332P/387I/390A/393G/536K/622D/681A/732W/749F/750D/775G/1148N/1248 D/1377D/1439E, 63Q/171P/260T/306G/332P/387I/390A/393G/536K/558L/732W/749F/750D/775G/1148N/1248D/1377D/1439E, 63Q/171P/211R/260T/306G/332P/387I/390A/393G/536R/555G/617G/749F/750D/775G/1148N/1248D/1377 D/1439E, 63Q/171P/211R/260T/306G/332P/387I/390A/393G/681R/749F/750D/775G/1148N/1248D/1377D/1439E, 63Q/171P/211R/260T/306G/332P/387I/390A/393G/536K/555E/732W/749F/750D/775G/1148N/1248D/1377 D/1439E, 63Q/171P/260T/306G/332P/387I/390A/393G/536R/732W/749F/750D/775G/1148N/1248D/1377D/1439E, 63Q/171P/260T/306G/332P/387I/390A/393G/555E/732W/749F/750D/775G/1148N/1248D/1377D/1439E, 63Q/171P/211R/260T/306G/332P/387I/390A/393G/536P/732W/749F/750D/775G/1148N/1248D/1377D/1439E, 63Q/171P/211R/212P/260T/306G/332P/387I/390A/393 G/732W/749F/750D/775G/1148N/1248D/1377D/1439E, 63Q/171P/211R/260T/306G/332P/387I/390A/393G/536K/732W/749F/750D/775G/1148N/1248D/1377D/1439E, 63Q/171P/211R/260T/306G/332P/387I/390A/393G/536K/617G/681A/749F/750D/775G/1148N/1248D/1377 D/1439E, 63Q/171P/211R/260T/306G/332P/387I/390A/393G/732W/749F/750D/775G/1148N/1248D/1377D/1439E, 63Q/171P/211R/260T/306G/332P/387I/390A/393G/536P/558L/617G/681A/732W/749F/750D/775G/1148N/1248D/1377D/1439E, 63Q/171P/260T/306G/332P/387I/390A/393G/555G/749F/750D/775G/1148N/1248D/1377D/1439E, 63Q/171P/260T/306G/332P/387I/390A/393G/536P/555E/558L/617G/749F/750D/775G/1148N/1248D/1377D/1439E, 63Q/171P/260T/306G/332P/387I/390A/393G/536K/617G/681A/749F/750D/775G/1148N/1248D/1377D/1439E, 63Q/171P/260T/306G/332P/387I/390A/393G/536K/555G/732W/749F/750D/775G/1148N/1248D/1377D/1439E, 63Q/171P/260T/306G/332P/387I/390A/393G/536P/622D/681A/732W/749F/750D/775G/1148N/1248D/1377 D/1439E, 63Q/171P/260T/306G/332P/387I/390A/393G/749F/750D/775G/1148N/1248D/1377D/1439E, 63Q/171P/212A/260T/306G/320Q/332P/387I/390A/393G/533R/617E/681G/732W/749F/750D/775G/1148N/1248D/1377D/1439E, 63Q/171P/211K/260T/306G/312L/332P/387I/390A/393G/533R/534I/617D/619E/681N/732W/749F/750D/775G/839K/1148N/1248D/1377D/1439E, 63Q/171P/212A/260T/306G/332P/387I/390A/393G/732W/749F/750D/775G/1148N/1248D/1377D/1439E, 63Q/171P/211K/212A/260T/306G/320Q/332P/387I/390A/393G/619E/681G/732W/749F/750D/775G/839K/1148N/1248D/1377D/1439E, 63Q/171P/260T/306G/332P/387I/390A/393G/732W/749F/750D/775G/797W/839K/1148N/1248D/1377D/1439E, 63Q/171P/212A/260T/306G/312L/320Q/332P/387I/390A/393G/533R/534I/617E/681G/732W/749F/750D/775G/1148N/1248D/1377D/1439E, 63Q/171P/260T/306G/332P/387I/390A/393G/558W/619E/681G/732W/749F/750D/775G/1148N/1248D/1377 D/1439E, 63Q/171P/260T/306G/320Q/332P/387I/390A/393G/558W/681G/732W/749F/750D/775G/1148N/1248D/1377D/1439E, 63Q/171P/212A/260T/306G/320Q/332P/387I/390A/393G/534I/619E/681G/732W/749F/750D/775G/797W/839K/1148N/1248D/1377D/1439E, 63Q/171P/260T/306G/320R/332P/387I/390A/393G/558W/619E/749F/750D/775G/839K/1148N/1248D/1377 D/1439E, 63Q/171P/211K/212A/260T/306G/312L/332P/387I/390A/393G/534I/558W/619E/681N/749F/750D/775G/839K/1148N/1248D/1377D/1439E, 63Q/171P/260T/306G/320R/332P/387I/390A/393G/534I/558R/619E/749F/750D/775G/797W/1148N/1248D/1377D/1439E, 63Q/171P/260T/306G/332P/387I/390A/393G/558W/617D/619E/681N/732W/749F/750D/775G/1148N/1248 D/1377D/1439E, 63Q/171P/211Q/260T/306G/332P/387I/390A/393G/534I/558W/611E/681N/732W/749F/750D/775G/1148N/1248D/1377D/1439E, 63Q/171P/211K/260T/306G/320R/332P/387I/390A/393G/617E/619E/681G/732W/749F/750D/775G/1148N/1248D/1377D/1439E, 63Q/171P/260T/306G/332P/387I/390A/393G/558W/617D/681G/732W/749F/750D/775G/797W/1148N/1248 D/1377D/1439E, 63Q/171P/260T/306G/320R/332P/387I/390A/393G/534I/619E/749F/750D/775G/1148N/1248D/1377D/1439E, 63Q/171P/211K/260T/306G/332P/387I/390A/393G/619E/681N/732W/749F/750D/775G/1148N/1248D/1377 D/1439E, 63Q/171P/211K/212A/260T/306G/332P/387I/390A/393G/534I/558W/619E/749F/750D/775G/839K/1148N/1248D/1377D/1439E, 63Q/171P/212A/260T/306G/320R/332P/387I/390A/393G/619E/681N/732W/749F/750D/775G/797W/839K/1148N/1248D/1377D/1439E, 63Q/171P/212A/260T/306G/320R/332P/387I/390A/393G/611E/732W/749F/750D/775G/839K/1148N/1248D/1377D/1439E, 63Q/171P/260T/306G/320Q/332P/387I/390A/393G/558W/619E/681N/732W/749F/750D/775G/1148N/1248 D/1377D/1439E, 63Q/171P/260T/306G/312L/332P/387I/390A/393G/533R/617D/681N/732W/749F/750D/775G/797W/839K/1148N/1248D/1377D/1439E, 63Q/171P/260T/306G/332P/387I/390A/393G/558W/611E/681N/732W/749F/750D/775G/1148N/1248D/1377 D/1439E, 63Q/171P/260T/306G/312L/332P/387I/390A/393G/732W/749F/750D/775G/1148N/1248D/1377D/1439E, 63Q/171P/211Q/260T/306G/320R/332P/387I/390A/393G/558W/619E/681G/749F/750D/775G/797W/839K/1148N/1248D/1377D/1439E, 63Q/171P/260T/306G/320Q/332P/387I/390A/393G/558W/749F/750D/775G/797W/1148N/1248D/1377D/1439E, 63Q/171P/260T/306G/332P/387I/390A/393G/533R/558W/611E/619E/716L/749F/750D/775G/839K/1148N/1248D/1377D/1439E, 63Q/171P/260T/306G/312L/332P/387I/390A/393G/681G/732W/749F/750D/775G/1148N/1248D/1377D/1439E, 63Q/171P/260T/306G/320Q/332P/387I/390A/393G/534I/619E/681N/732W/749F/750D/775G/1148N/1248D/1377D/1439E, 63Q/171P/211K/260T/306G/320Q/332P/387I/390A/393G/533R/536L/619E/732W/749F/750D/775G/1148N/1248D/1377D/1439E, 63Q/171P/260T/306G/332P/387I/390A/393G/558W/617E/681N/732W/749F/750D/775G/839K/1148N/1248 D/1377D/1439E, 63Q/171P/212A/260T/306G/320R/332P/387I/390A/393G/534I/558W/681G/732W/749F/750D/775G/1148N/1248D/1377D/1439E, 63Q/171P/260T/306G/332P/387I/390A/393G/533R/534I/681N/732W/749F/750D/775G/839K/1148N/1248D/1377D/1439E, 63Q/171P/212A/260T/306G/332P/387I/390A/393G/534I/619E/681G/732W/749F/750D/775G/1148N/1248D/1377D/1439E, 63Q/171P/260T/306G/332P/387I/390A/393G/617E/619E/681G/732W/749F/750D/775G/1148N/1248D/1377 D/1439E, 63Q/171P/211Q/212A/260T/306G/320R/332P/387I/390A/393G/558W/617E/681N/732W/749F/750D/775G/1148N/1248D/1377D/1439E, 63Q/171P/212A/260T/306G/332P/387I/390A/393G/534I/558W/681N/732W/749F/750D/775G/797W/1148N/1248D/1377D/1439E, 63Q/171P/212A/260T/306G/320R/332P/387I/390A/393G/534I/732W/749F/750D/775G/1148N/1248D/1377 D/1439E, 63Q/171P/211K/260T/306G/312L/332P/387I/390A/393G/619E/732W/749F/750D/775G/1148N/1248D/1377 D/1439E, 63Q/171P/212A/260T/306G/312L/332P/387I/390A/393G/619E/732W/749F/750D/775G/839K/1148N/1248D/1377D/1439E, 63Q/171P/210P/212A/260T/306G/320R/332P/387I/390A/393G/558W/681G/732W/749F/750D/775G/1148N/1248D/1377D/1439E, 63Q/171P/212A/260T/306G/312L/332P/387I/390A/393G/534I/681N/732W/749F/750D/775G/797W/839K/1148N/1248D/1377D/1439E, 63Q/171P/211K/260T/306G/320R/332P/387I/390A/393G/558W/732W/749F/750D/775G/839K/1148N/1248 D/1377D/1439E, 63Q/171P/260T/306G/332P/387I/390A/393G/732W/749F/750D/775G/924Q/1148N/1248D/1377D/1439E, 63Q/171P/260T/306G/332P/387I/390A/393G/732W/749F/750D/775G/916L/1148N/1248D/1377D/1439E, 63Q/171P/260T/306G/332P/387I/390A/393G/732W/749F/750D/757T/775G/1148N/1248D/1377D/1439E, 63Q/171P/260T/306G/332P/387I/390A/393G/732W/737K/749F/750D/775G/1148N/1248D/1377D/1439E, 63Q/171P/260T/306G/332P/387I/390A/393G/732W/749F/750D/775G/818V/1148N/1248D/1377D/1439E, 63Q/171P/260T/306G/332P/387I/390A/393G/732W/749F/750D/775G/801G/1148N/1248D/1377D/1439E, 63Q/171P/260T/294G/306G/332P/387I/390A/393G/732W/749F/750D/775G/1148N/1248D/1377D/1439E, 63Q/171P/260T/306G/332P/387I/390A/393G/732W/749F/750D/775G/799S/1148N/1248D/1377D/1439E, 63Q/171P/260T/306G/332P/387I/390A/393G/732W/749F/750D/775G/799E/1148N/1248D/1377D/1439E, 63Q/171P/260T/306G/332P/387I/390A/393G/732W/749F/750D/775G/809G/1148N/1248D/1377D/1439E, 63Q/171P/260T/306G/332P/387I/390A/393G/732W/749F/750D/775G/791M/1148N/1248D/1377D/1439E, 63Q/171P/260T/306G/332P/387I/390A/393G/732W/737V/749F/750D/775G/1148N/1248D/1377D/1439E, 63Q/171P/260T/306G/332P/387I/390A/393G/732W/749F/750D/775G/805Q/1148N/1248D/1377D/1439E, 63Q/171P/260T/306G/332P/387I/390A/393G/732W/749F/750D/775G/799Q/1148N/1248D/1377D/1439E, 63Q/171P/260T/306G/332P/387I/390A/393G/732W/737S/749F/750D/775G/1148N/1248D/1377D/1439E, 63Q/171P/260T/306G/332P/387I/390A/393G/732W/749F/750D/775G/916M/1148N/1248D/1377D/1439E, 63Q/171P/260T/306G/332P/387I/390A/393G/732W/749F/750D/759S/775G/1148N/1248D/1377D/1439E, 63Q/171P/260T/306G/332P/387I/390A/393G/732W/749F/750D/775G/799V/1148N/1248D/1377D/1439E, 63Q/171P/260T/306G/332P/387I/390A/393G/538V/732W/749F/750D/775G/1148N/1248D/1377D/1439E, 63Q/171P/260T/306G/332P/387I/390A/393G/732W/749F/750D/775G/1109G/1148N/1248D/1377D/1439E, 63Q/168R/171P/260T/306G/332P/387I/390A/393G/732W/749F/750D/775G/1148N/1248D/1377D/1439E, 63Q/171P/260T/306G/332P/387I/390A/393G/732W/749F/750D/775G/921R/1148N/1248D/1377D/1439E, 63Q/171P/260T/306G/332P/387I/390A/393G/732W/749F/750D/775G/926T/1148N/1248D/1377D/1439E, 63Q/171P/260T/306G/332P/387I/390A/393G/732W/749F/750D/775G/916T/1148N/1248D/1377D/1439E, 63Q/171P/260T/306G/332P/387I/390A/393G/732W/737G/749F/750D/775G/1148N/1248D/1377D/1439E, or 63Q/171P/260T/306G/332P/387I/390A/393G/732W/749F/750D/775G/801R/1148N/1248D/1377D/1439E, wherein the amino acid positions are relative to the reference sequence corresponding to SEQ ID NO: 2.

In some embodiments, the amino acid sequence of the recombinant factor VIII polypeptide comprises at least a substitution or substitution set at amino acid position(s) 63/171/212/260/306/320/332/387/390/393/534/558/681/732/749/750/775/1042/1148/1248/1377/1439, 63/171/212/260/306/320/332/387/390/393/534/558/681/732/749/750/775/1041/1148/1248/1377/1439, 63/171/212/260/306/320/332/387/390/393/534/558/681/732/749/750/775/1051/1148/1248/1377/1439, 63/171/212/260/306/320/332/387/390/393/534/558/681/732/749/750/775/1148/1248/1283/1377/1439, 63/171/212/260/306/320/332/387/390/393/534/558/681/732/749/750/775/1148/1248/1281/1377/1439, 63/171/212/260/306/320/332/387/390/393/534/558/681/732/749/750/775/916/1148/1248/1377/1439, 63/171/187/212/260/306/320/332/387/390/393/534/558/681/732/749/750/775/1148/1248/1377/1439, 63/171/212/215/260/306/320/332/387/390/393/534/558/681/732/749/750/775/1148/1248/1377/1439, 63/171/178/212/260/306/320/332/387/390/393/534/558/681/732/749/750/775/1148/1248/1377/1439, 63/171/212/260/306/320/332/387/390/393/534/558/681/732/749/750/775/1050/1148/1248/1377/1439, 63/171/212/260/306/320/332/387/390/393/534/558/681/732/749/750/775/1148/1210/1248/1377/1439, 63/104/171/212/260/306/320/332/387/390/393/534/558/681/732/749/750/775/1148/1248/1377/1439, 63/171/212/260/306/320/332/387/390/393/534/558/615/681/732/749/750/775/1148/1248/1377/1439, 63/171/212/260/306/320/332/387/390/393/534/558/610/681/732/749/750/775/1148/1248/1377/1439, 63/171/212/260/306/320/332/387/390/393/534/536/558/681/732/749/750/775/1148/1248/1377/1439, 63/171/212/260/306/320/332/387/390/393/534/558/618/681/732/749/750/775/1148/1248/1377/1439, 63/171/212/260/306/320/332/387/390/393/534/558/621/681/732/749/750/775/1148/1248/1377/1439, 63/171/212/260/306/320/332/387/390/393/534/558/614/681/732/749/750/775/1148/1248/1377/1439, 63/110/171/212/260/306/320/332/387/390/393/534/558/681/732/749/750/775/1148/1248/1377/1439, 63/171/212/260/301/306/320/332/387/390/393/534/558/681/732/749/750/775/1148/1248/1377/1439, 63/171/212/260/306/320/332/387/390/393/534/558/681/732/749/750/775/1078/1148/1248/1377/1439, 63/171/212/260/306/320/332/387/390/393/534/558/681/732/749/750/775/812/1148/1248/1377/1439, 63/171/212/260/306/320/332/387/390/393/534/558/681/732/749/750/775/786/1148/1248/1377/1439, 63/127/171/212/260/306/320/332/387/390/393/534/558/681/732/749/750/775/1148/1248/1377/1439, 63/171/175/212/260/306/320/332/387/390/393/534/558/681/732/749/750/775/1148/1248/1377/1439, 63/171/212/260/306/320/332/387/390/393/534/558/681/732/749/750/775/1093/1148/1248/1377/1439, 63/171/212/260/306/320/332/387/390/393/534/558/681/732/749/750/775/1088/1148/1248/1377/1439, 63/171/212/260/306/320/332/387/390/393/534/558/650/681/732/749/750/775/1148/1248/1377/1439, 63/171/212/260/306/320/332/387/390/393/534/535/558/681/732/749/750/775/1148/1248/1377/1439, 63/171/212/260/306/320/332/387/390/393/534/558/681/732/749/750/775/806/1148/1248/1377/1439, 63/171/212/260/306/320/332/387/390/393/534/558/681/732/738/749/750/775/1148/1248/1377/1439, 63/171/212/260/306/320/332/387/390/393/534/558/681/700/732/749/750/775/1148/1248/1377/1439, 63/132/171/212/260/306/320/332/387/390/393/534/558/681/732/749/750/775/1148/1248/1377/1439, 63/171/212/260/306/320/332/387/390/393/534/558/681/732/749/750/775/1076/1148/1248/1377/1439, 63/171/212/260/306/320/332/387/390/393/534/558/681/732/749/750/775/1084/1148/1248/1377/1439, 63/171/212/260/306/320/332/387/390/393/498/534/558/681/732/749/750/775/1148/1248/1377/1439, 63/171/212/260/306/320/332/387/390/393/534/558/681/732/749/750/756/775/1148/1248/1377/1439, 63/171/212/260/306/320/332/387/390/393/534/558/681/732/749/750/775/1108/1148/1248/1377/1439, 63/171/212/260/306/320/332/387/390/393/534/558/681/732/746/749/750/775/1148/1248/1377/1439, 63/160/171/212/260/306/320/332/387/390/393/534/558/681/732/749/750/775/1148/1248/1377/1439, 63/171/212/260/306/320/332/387/390/393/534/543/558/681/732/749/750/775/1148/1248/1377/1439, 63/171/212/260/306/310/320/332/387/390/393/534/558/681/732/749/750/775/1148/1248/1377/1439, 63/171/212/260/306/320/332/387/390/393/534/558/584/681/732/749/750/775/1148/1248/1377/1439, 63/171/212/260/306/320/332/387/390/393/534/558/681/732/749/750/775/918/1148/1248/1377/1439, 63/171/212/260/306/320/332/387/390/393/534/558/681/732/749/750/775/1148/1248/1377/1439/1452, 63/171/212/260/306/320/332/387/390/393/534/558/681/732/749/750/775/790/1148/1248/1377/1439, 63/171/212/260/292/306/320/332/387/390/393/534/558/681/732/749/750/775/1148/1248/1377/1439, 63/171/212/260/306/320/332/387/390/393/534/558/681/732/749/750/775/800/1148/1248/1377/1439, 63/167/171/212/260/306/320/332/387/390/393/534/558/681/732/749/750/775/1148/1248/1377/1439, 63/171/212/260/306/320/332/387/390/393/534/558/681/732/749/750/775/892/1148/1248/1377/1439, 63/171/212/260/304/306/320/332/387/390/393/534/558/681/732/749/750/775/1148/1248/1377/1439, 63/171/212/260/306/320/332/387/390/393/534/558/681/732/749/750/775/1116/1148/1248/1377/1439, 63/171/212/260/306/320/332/387/390/393/534/558/681/732/749/750/752/775/1148/1248/1377/1439, 63/171/212/260/281/306/320/332/387/390/393/534/558/681/732/749/750/775/1148/1248/1377/1439, 63/171/212/260/306/320/332/387/390/393/534/558/681/732/749/750/775/917/1148/1248/1377/1439, 63/171/212/260/306/320/332/387/390/393/534/558/681/712/732/749/750/775/1148/1248/1377/1439, 63/171/212/260/306/320/332/387/390/393/534/558/681/732/749/750/775/1080/1148/1248/1377/1439, 63/171/212/260/306/320/332/387/390/393/534/558/681/732/749/750/775/806/1148/1248/1352/1377/1439, 63/171/212/260/306/320/332/387/390/393/534/558/681/732/749/750/775/1095/1148/1248/1377/1439, 63/171/212/260/306/320/332/387/390/393/534/558/681/732/744/749/750/775/1148/1248/1377/1439, 63/171/212/260/306/320/332/387/390/393/534/558/681/732/749/750/775/1098/1148/1248/1377/1439, 63/171/212/260/306/320/332/387/390/393/534/558/681/732/749/750/775/948/1148/1248/1377/1439, 63/119/171/212/260/306/320/332/387/390/393/534/558/681/732/749/750/775/1148/1248/1377/1439, 63/171/212/260/306/320/332/387/390/393/534/558/681/732/749/750/775/785/1148/1248/1377/1439, 63/171/212/260/306/320/332/387/390/393/534/558/681/732/749/750/775/1113/1148/1248/1377/1439, 63/171/212/260/306/320/332/387/390/393/534/558/667/681/732/749/750/775/1148/1248/1377/1439, 63/158/171/212/260/306/320/332/387/390/393/534/558/681/732/749/750/775/1148/1248/1377/1439, 63/171/212/260/306/320/332/387/390/393/534/558/580/681/732/749/750/775/1148/1248/1377/1439, 63/171/212/260/306/320/332/387/390/393/534/558/681/707/732/749/750/775/1148/1248/1377/1439, 63/171/212/260/306/320/332/387/390/393/534/558/681/731/732/749/750/775/1148/1248/1377/1439, 63/171/212/260/306/320/332/387/390/393/534/558/681/732/749/750/775/1100/1148/1248/1377/1439, 63/171/212/260/306/320/332/387/390/393/534/558/681/732/749/750/775/1089/1148/1248/1377/1439, 63/171/212/260/306/320/332/387/390/393/534/558/681/732/749/750/775/1092/1148/1248/1377/1439, 63/171/212/260/306/320/332/387/390/393/534/558/681/732/749/750/775/939/1148/1248/1377/1439, 63/171/212/260/306/320/332/387/390/393/534/558/681/732/749/750/775/1104/1148/1248/1377/1439, 63/171/212/260/306/320/332/387/390/393/534/558/681/732/736/749/750/775/1148/1248/1377/1439, 63/171/212/260/306/320/332/387/390/393/534/558/681/732/749/750/775/1148/1248/1297/1377/1439, 63/171/212/260/306/320/332/387/390/393/534/558/681/732/749/750/775/1127/1148/1248/1377/1439, 63/171/212/260/306/320/332/387/390/393/534/558/681/686/732/749/750/775/1148/1248/1377/1439, 63/171/212/260/306/320/332/387/390/393/534/558/681/732/749/750/775/1148/1129/1248/1377/1439, 63/171/212/260/306/320/332/387/390/393/534/558/681/732/749/750/775/1138/1148/1248/1377/1439, 63/171/212/260/306/320/332/387/390/393/534/558/681/732/749/750/775/786/821/1148/1248/1377/1439, 63/171/212/260/306/320/332/387/390/393/534/558/680/681/732/749/750/775/1148/1248/1377/1439, 63/171/212/260/306/132/320/332/387/390/393/534/558/681/732/749/750/775/1148/1248/1377/1439, 63/171/212/260/306/320/332/387/390/393/534/558/681/732/749/750/775/1101/1148/1248/1377/1439, 63/171/212/260/306/320/332/387/390/393/534/558/681/732/749/750/775/808/1148/1248/1377/1439, 63/171/212/260/306/320/332/387/390/393/534/558/681/732/739/749/750/775/1148/1248/1377/1439, 63/171/212/260/306/308/320/332/387/390/393/534/558/681/732/749/750/775/1148/1248/1377/1439, 63/171/212/260/306/320/332/387/390/393/534/536/555/558/619/681/749/750/775/799/805/1148/1248/1377/1439, 63/171/212/260/306/320/332/387/390/393/534/555/558/619/681/732/749/750/775/801/1148/1248/1377/1439, 63/171/212/260/306/320/332/387/390/393/534/555/558/619/681/732/749/750/775/799/1148/1248/1377/1439, 63/171/212/260/306/320/332/387/390/393/534/558/619/681/732/749/750/775/799/801/805/1148/1248/1377/1439, 63/171/212/260/306/320/332/387/390/393/534/536/555/558/619/681/749/750/775/799/1148/1248/1377/1439, 63/171/212/260/306/320/332/387/390/393/534/558/681/732/749/750/775/799/805/1148/1248/1377/1439, 63/171/212/260/306/320/332/387/390/393/534/558/681/732/749/750/775/801/1148/1248/1377/1439, 63/171/212/246/260/306/320/332/387/390/393/534/558/619/681/732/749/750/775/799/801/1148/1248/1377/1439, 63/171/212/260/306/320/332/387/390/393/534/536/555/558/619/681/732/749/750/775/1148/1248/1377/1439, 63/171/212/260/306/320/332/387/390/393/534/558/619/681/732/749/750/775/839/1148/1248/1377/1439, 63/171/212/260/306/320/332/387/390/393/534/558/619/681/732/749/750/775/799/805/1148/1248/1377/1439, 63/171/212/260/306/320/332/387/390/393/534/536/558/681/732/749/750/775/801/805/1148/1248/1377/1439, 63/171/212/260/306/320/332/387/390/393/534/555/558/619/681/732/749/750/775/799/805/1148/1248/1377/1439, 63/171/212/260/306/320/332/387/390/393/534/558/619/681/732/749/750/775/801/805/1148/1248/1377/1439, 63/171/212/260/306/320/332/387/390/393/534/555/558/619/681/732/749/750/775/1148/1248/1377/1439, 63/171/212/260/306/320/332/387/390/393/534/536/558/619/681/732/749/750/775/801/1148/1248/1377/1439, 63/171/212/260/306/320/332/387/390/393/534/558/681/732/749/750/775/801/805/1148/1248/1377/1439, 63/171/212/260/306/320/332/387/390/393/534/536/558/619/681/732/749/750/775/799/805/1148/1248/1377/1439, 63/171/212/260/306/320/332/387/390/393/534/555/558/619/681/749/750/775/799/801/805/1148/1248/1377/1439, 63/171/212/260/306/320/332/387/390/393/534/558/681/732/750/775/1148/1248/1377/1439, 63/171/212/260/306/320/332/387/390/393/534/558/616/681/732/749/750/775/1148/1248/1377/1439, 63/171/212/260/306/320/332/387/390/393/534/558/611/681/732/749/750/775/1148/1248/1377/1439, 63/171/212/260/306/320/332/370/387/390/393/534/558/614/681/732/749/750/775/1148/1248/1377/1439, 63/171/212/260/306/320/332/387/390/393/534/558/681/732/749/750/775/1148/1156/1248/1377/1439, 63/171/212/260/306/320/332/387/390/393/534/558/681/732/749/750/775/1148/1158/1248/1377/1439, 63/171/212/260/306/320/332/387/390/393/534/555/558/681/732/737/749/750/775/799/1148/1248/1377/1439, 63/171/212/260/306/320/332/387/390/393/534/536/558/619/681/732/749/750/775/809/1109/1148/1248/1377/1439, 63/171/212/260/306/320/332/387/390/393/534/536/555/558/619/681/732/737/749/750/775/799/809/839/916/921/1148/1248/1377/1439, 63/171/212/260/306/320/332/387/390/393/534/558/681/732/749/750/775/916/1109/1148/1248/1377/1439, 63/171/212/260/306/320/332/387/390/393/534/536/558/619/681/732/749/750/775/799/916/1109/1148/1248/1377/1439, 63/171/212/260/306/320/332/387/390/393/534/555/558/619/681/732/749/750/775/807/1109/1148/1248/1377/1439, 63/171/212/260/306/320/332/387/390/393/534/555/558/619/681/749/750/775/799/1148/1248/1377/1439, 63/171/212/260/306/320/332/387/390/393/534/558/619/681/732/749/750/775/799/916/1109/1148/1248/1377/1439, 63/171/212/260/306/320/332/387/390/393/534/558/619/681/732/749/750/775/1148/1248/1377/1439, 63/171/212/260/306/320/332/387/390/393/534/555/558/681/732/749/750/775/921/1148/1248/1377/1439, 63/171/212/260/306/320/332/387/390/393/534/558/619/681/732/749/750/775/916/1148/1248/1377/1439, 63/171/212/260/306/320/332/387/390/393/534/555/558/681/732/749/750/775/1148/1248/1377/1439, 63/171/212/260/306/320/332/387/390/393/534/558/681/749/750/775/1148/1248/1377/1439, 63/171/212/260/306/320/332/387/390/393/534/558/619/681/732/749/750/775/799/1148/1248/1377/1439, 63/171/212/260/306/320/332/387/390/393/534/558/681/732/749/750/775/1148/1248/1377/1439, 63/171/212/260/306/320/332/387/390/393/534/555/558/681/732/749/750/775/916/1148/1248/1377/1439, 63/171/212/260/306/320/332/387/390/393/534/558/681/732/749/750/775/839/1148/1248/1377/1439, or 63/171/212/260/306/320/332/387/390/393/534/558/681/732/749/750/775/799/1148/1248/1377/1439, wherein the amino acid positions are relative to the reference sequence corresponding to SEQ ID NO: 2.

In some embodiments, the amino acid sequence of the recombinant factor VIII polypeptide comprises at least a substitution or substitution set 63Q/171P/212A/260T/306G/320R/332P/387I/390A/393G/534I/558W/681G/732W/749F/750D/775G/1042E/1148N/1248D/1377D/1439E, 63Q/171P/212A/260T/306G/320R/332P/387I/390A/393G/534I/558W/681G/732W/749F/750D/775G/1041N/1148N/1248D/1377D/1439E, 63Q/171P/212A/260T/306G/320R/332P/387I/390A/393G/534I/558W/681G/732W/749F/750D/775G/1051H/1148N/1248D/1377D/1439E, 63Q/171P/212A/260T/306G/320R/332P/387I/390A/393G/534I/558W/681G/732W/749F/750D/775G/1148N/1248D/1283H/1377D/1439E, 63Q/171P/212A/260T/306G/320R/332P/387I/390A/393G/534I/558W/681G/732W/749F/750D/775G/1148N/1248D/1283T/1377D/1439E, 63Q/171P/212A/260T/306G/320R/332P/387I/390A/393G/534I/558W/681G/732W/749F/750D/775G/1148N/1248D/1281Q/1377D/1439E, 63Q/171P/212A/260T/306G/320R/332P/387I/390A/393G/534I/558W/681G/732W/749F/750D/775G/1148N/1248D/1281P/1377D/1439E, 63Q/171P/212A/260T/306G/320R/332P/387I/390A/393G/534I/558W/681G/732W/749F/750D/775G/916E/1148N/1248D/1377D/1439E, 63Q/171P/212A/260T/306G/320R/332P/387I/390A/393G/534I/558W/681G/732W/749F/750D/775G/1051W/1148N/1248D/1377D/1439E, 63Q/171P/187C/212A/260T/306G/320R/332P/387I/390A/393G/534I/558W/681G/732W/749F/750D/775G/1148N/1248D/1377D/1439E, 63Q/171P/212A/215E/260T/306G/320R/332P/387I/390A/393G/534I/558W/681G/732W/749F/750D/775G/1148N/1248D/1377D/1439E, 63Q/171P/212A/215P/260T/306G/320R/332P/387I/390A/393G/534I/558W/681G/732W/749F/750D/775G/1148N/1248D/1377D/1439E, 63Q/171P/212A/215S/260T/306G/320R/332P/387I/390A/393G/534I/558W/681G/732W/749F/750D/775G/1148N/1248D/1377D/1439E, 63Q/171P/178C/212A/260T/306G/320R/332P/387I/390A/393G/534I/558W/681G/732W/749F/750D/775G/1148N/1248D/1377D/1439E, 63Q/171P/212A/260T/306G/320R/332P/387I/390A/393G/534I/558W/681G/732W/749F/750D/775G/1050T/1148N/1248D/1377D/1439E, 63Q/171P/212A/260T/306G/320R/332P/387I/390A/393G/534I/558W/681G/732W/749F/750D/775G/1148N/1210T/1248D/1377D/1439E, 63Q/171P/212A/260T/306G/320R/332P/387I/390A/393G/534I/558W/681G/732W/749F/750D/775G/1148N/1248D/1281K/1377D/1439E, 63Q/171P/212A/260T/306G/320R/332P/387I/390A/393G/534I/558W/681G/732W/749F/750D/775G/1042A/1148N/1248D/1377D/1439E, 63Q/104S/171P/212A/260T/306G/320R/332P/387I/390A/393G/534I/558W/681G/732W/749F/750D/775G/1148N/1248D/1377D/1439E, 63Q/171P/212A/260T/306G/320R/332P/387I/390A/393G/534I/558W/615D/681G/732W/749F/750D/775G/1148N/1248D/1377D/1439E, 63Q/171P/212A/260T/306G/320R/332P/387I/390A/393G/534I/558W/610S/681G/732W/749F/750D/775G/1148N/1248D/1377D/1439E, 63Q/171P/212A/260T/306G/320R/332P/387I/390A/393G/534I/558W/610K/681G/732W/749F/750D/775G/1148N/1248D/1377D/1439E, 63Q/171P/212A/260T/306G/320R/332P/387I/390A/393G/534I/558W/610T/681G/732W/749F/750D/775G/1148N/1248D/1377D/1439E, 63Q/171P/212A/260T/306G/320R/332P/387I/390A/393G/534I/536P/558W/681G/732W/749F/750D/775G/1148N/1248D/1377D/1439E, 63Q/171P/212A/260T/306G/320R/332P/387I/390A/393G/534I/558W/618Y/681G/732W/749F/750D/775G/1148N/1248D/1377D/1439E, 63Q/171P/212A/260T/306G/320R/332P/387I/390A/393G/534I/558W/621W/681G/732W/749F/750D/775G/1148N/1248D/1377D/1439E, 63Q/171P/212A/260T/306G/320R/332P/387I/390A/393G/534I/558W/618P/681G/732W/749F/750D/775G/1148N/1248D/1377D/1439E, 63Q/171P/212A/260T/306G/320R/332P/387I/390A/393G/534I/558W/614F/681G/732W/749F/750D/775G/1148N/1248D/1377D/1439E, 63Q/110H/171P/212A/260T/306G/320R/332P/387I/390A/393G/534I/558W/681G/732W/749F/750D/775G/1148N/1248D/1377D/1439E, 63Q/171P/212A/260T/301K/306G/320R/332P/387I/390A/393G/534I/558W/681G/732W/749F/750D/775G/1148N/1248D/1377D/1439E, 63Q/171P/212A/260T/306G/320R/332P/387I/390A/393G/534I/558W/681G/732W/749F/750D/775G/1078S/1148N/1248D/1377D/1439E, 63Q/171P/212A/260T/306G/320R/332P/387I/390A/393G/534I/558W/681G/732W/749F/750D/775G/812G/1148N/1248D/1377D/1439E, 63Q/171P/212A/260T/306G/320R/332P/387I/390A/393G/534I/558W/681G/732W/749F/750D/775G/786C/1148N/1248D/1377D/1439E, 63Q/127V/171P/212A/260T/306G/320R/332P/387I/390A/393G/534I/558W/681G/732W/749F/750D/775G/1148N/1248D/1377D/1439E, 63Q/171P/175G/212A/260T/306G/320R/332P/387I/390A/393G/534I/558W/681G/732W/749F/750D/775G/1148N/1248D/1377D/1439E, 63Q/171P/212A/260T/306G/320R/332P/387I/390A/393G/534I/558W/681G/732W/749F/750D/775G/1093W/1148N/1248D/1377D/1439E, 63Q/171P/212A/260T/306G/320R/332P/387I/390A/393G/534I/558W/681G/732W/749F/750D/775G/1088G/1148N/1248D/1377D/1439E, 63Q/171P/212A/260T/306G/320R/332P/387I/390A/393G/534I/558W/681G/732W/749F/750D/775G/1093T/1148N/1248D/1377D/1439E, 63Q/171P/212A/260T/306G/320R/332P/387I/390A/393G/534I/558W/650M/681G/732W/749F/750D/775G/1148N/1248D/1377D/1439E, 63Q/171P/212A/260T/301M/306G/320R/332P/387I/390A/393G/534I/558W/681G/732W/749F/750D/775G/1148N/1248D/1377D/1439E, 63Q/171P/212A/260T/306G/320R/332P/387I/390A/393G/534I/535N/558W/681G/732W/749F/750D/775G/1148N/1248D/1377D/1439E, 63Q/171P/212A/260T/306G/320R/332P/387I/390A/393G/534I/558W/681G/732W/749F/750D/775G/806R/1148N/1248D/1377D/1439E, 63Q/171P/212A/260T/306G/320R/332P/387I/390A/393G/534I/558W/681G/732W/738E/749F/750D/775G/1148N/1248D/1377D/1439E, 63Q/171P/212A/260T/306G/320R/332P/387I/390A/393G/534I/558W/681G/700E/732W/749F/750D/775G/1148N/1248D/1377D/1439E, 63Q/132R/171P/212A/260T/306G/320R/332P/387I/390A/393G/534I/558W/681G/732W/749F/750D/775G/1148N/1248D/1377D/1439E, 63Q/171P/212A/260T/306G/320R/332P/387I/390A/393G/534I/558W/681G/732W/749F/750D/775G/1076G/1148N/1248D/1377D/1439E, 63Q/171P/212A/260T/306G/320R/332P/387I/390A/393G/534I/558W/681G/732W/749F/750D/775G/1084L/1148N/1248D/1377D/1439E, 63Q/171P/212A/260T/306G/320R/332P/387I/390A/393G/498A/534I/558W/681G/732W/749F/750D/775G/1148N/1248D/1377D/1439E, 63Q/171P/212A/260T/306G/320R/332P/387I/390A/393G/534I/558W/681G/732W/749F/750D/775G/786L/1148N/1248D/1377D/1439E, 63Q/171P/212A/260T/306G/320R/332P/387I/390A/393G/534I/558W/681G/732W/738F/749F/750D/775G/1148N/1248D/1377D/1439E, 63Q/171P/212A/260T/306G/320R/332P/387I/390A/393G/534I/535G/558W/681G/732W/749F/750D/775G/1148N/1248D/1377D/1439E, 63Q/171P/212A/260T/306G/320R/332P/387I/390A/393G/534I/558W/681G/732W/749F/750D/775G/806E/1148N/1248D/1377D/1439E, 63Q/171P/212A/260T/306G/320R/332P/387I/390A/393G/534I/558W/681G/732W/749F/750D/756G/775G/1148N/1248D/1377D/1439E, 63Q/171P/212A/260T/306G/320R/332P/387I/390A/393G/534I/558W/681G/732W/749F/750D/775G/1108S/1148N/1248D/1377D/1439E, 63Q/171P/212A/260T/306G/320R/332P/387I/390A/393G/534I/558W/681G/732W/749F/750D/775G/1093V/1148N/1248D/1377D/1439E, 63Q/171P/212A/260T/306G/320R/332P/387I/390A/393G/534I/558W/681G/732W/746P/749F/750D/775G/1148N/1248D/1377D/1439E, 63Q/160E/171P/212A/260T/306G/320R/332P/387I/390A/393G/534I/558W/681G/732W/749F/750D/775G/1148N/1248D/1377D/1439E, 63Q/160T/171P/212A/260T/306G/320R/332P/387I/390A/393G/534I/558W/681G/732W/749F/750D/775G/1148N/1248D/1377D/1439E, 63Q/171P/178V/212A/260T/306G/320R/332P/387I/390A/393G/534I/558W/681G/732W/749F/750D/775G/1148N/1248D/1377D/1439E, 63Q/171P/212A/260T/306G/320R/332P/387I/390A/393G/534I/543L/558W/681G/732W/749F/750D/775G/1148N/1248D/1377D/1439E, 63Q/171P/212A/260T/306G/310G/320R/332P/387I/390A/393G/534I/558W/681G/732W/749F/750D/775G/1148N/1248D/1377D/1439E, 63Q/171P/212A/260T/306G/320R/332P/387I/390A/393G/534I/558W/681G/700Q/732W/749F/750D/775G/1148N/1248D/1377D/1439E, 63Q/171P/212A/260T/306G/320R/332P/387I/390A/393G/534I/558W/681G/732W/749F/750D/775G/1093A/1148N/1248D/1377D/1439E, 63Q/171P/212A/260T/306G/320R/332P/387I/390A/393G/534I/558W/584A/681G/732W/749F/750D/775G/1148N/1248D/1377D/1439E, 63Q/171P/212A/260T/306G/320R/332P/387I/390A/393G/534I/558W/681G/732W/749F/750D/775G/918S/1148N/1248D/1377D/1439E, 63Q/171P/212A/260T/306G/320R/332P/387I/390A/393G/534I/558W/681G/732W/749F/750D/775G/1148N/1248D/1377D/1439E/1452L, 63Q/171P/212A/260T/306G/320R/332P/387I/390A/393G/534I/558W/681G/732W/738P/749F/750D/775G/1148N/1248D/1377D/1439E, 63Q/171P/212A/260T/306G/320R/332P/387I/390A/393G/534I/558W/681G/732W/749F/750D/775G/790W/1148N/1248D/1377D/1439E, 63Q/171P/212A/260T/306G/320R/332P/387I/390A/393G/534I/558W/681G/732W/749F/750D/775G/790I/1148N/1248D/1377D/1439E, 63Q/171P/212A/260T/292D/306G/320R/332P/387I/390A/393G/534I/558W/681G/732W/749F/750D/775G/1148N/1248D/1377D/1439E, 63Q/171P/212A/260T/306G/320R/332P/387I/390A/393G/534I/558W/681G/732W/749F/750D/775G/800M/1148N/1248D/1377D/1439E, 63Q/171P/212A/260T/306G/320R/332P/387I/390A/393G/534I/558W/681G/732W/749F/750D/775G/1108M/1148N/1248D/1377D/1439E, 63Q/171P/212A/260T/306G/320R/332P/387I/390A/393G/534I/558W/681G/732W/749F/750D/775G/1088S/1148N/1248D/1377D/1439E, 63Q/167G/171P/212A/260T/306G/320R/332P/387I/390A/393G/534I/558W/681G/732W/749F/750D/775G/1148N/1248D/1377D/1439E, 63Q/171P/212A/260T/306G/320R/332P/387I/390A/393G/534I/558W/681G/732W/749F/750D/775G/892T/1148N/1248D/1377D/1439E, 63Q/171P/212A/260T/306G/320R/332P/387I/390A/393G/534I/558W/681G/732W/749F/750D/775G/790P/1148N/1248D/1377D/1439E, 63Q/171P/212A/260T/306G/320R/332P/387I/390A/393G/534I/558W/681G/732W/749F/750D/775G/800R/1148N/1248D/1377D/1439E, 63Q/171P/212A/260T/306G/320R/332P/387I/390A/393G/534I/558W/681G/732W/738K/749F/750D/775G/1148N/1248D/1377D/1439E, 63Q/171P/212A/260T/304G/306G/320R/332P/387I/390A/393G/534I/558W/681G/732W/749F/750D/775G/1148N/1248D/1377D/1439E, 63Q/171P/212A/260T/306G/320R/332P/387I/390A/393G/534I/558W/681G/732W/749F/750D/775G/1116G/1148N/1248D/1377D/1439E, 63Q/171P/178Q/212A/260T/306G/320R/332P/387I/390A/393G/534I/558W/681G/732W/749F/750D/775G/1148N/1248D/1377D/1439E, 63Q/171P/212A/260T/306G/320R/332P/387I/390A/393G/534I/558W/681G/732W/749F/750D/775G/1116A/1148N/1248D/1377D/1439E, 63Q/171P/212A/260T/306G/320R/332P/387I/390A/393G/534I/558W/681G/732W/749F/750D/752G/775G/1148N/1248D/1377D/1439E, 63Q/171P/212A/260T/306G/320R/332P/387I/390A/393G/534I/558W/681G/732W/749F/750D/775G/1108W/1148N/1248D/1377D/1439E, 63Q/171P/212A/260T/306G/320R/332P/387I/390A/393G/534I/558W/681G/732W/738D/749F/750D/775G/1148N/1248D/1377D/1439E, 63Q/171P/212A/260T/281A/306G/320R/332P/387I/390A/393G/534I/558W/681G/732W/749F/750D/775G/1148N/1248D/1377D/1439E, 63Q/171P/212A/260T/306G/320R/332P/387I/390A/393G/534I/558W/681G/732W/749F/750D/775G/917G/1148N/1248D/1377D/1439E, 63Q/171P/212A/260T/306G/320R/332P/387I/390A/393G/534I/558W/681G/712M/732W/749F/750D/775G/1148N/1248D/1377D/1439E, 63Q/171P/212A/260T/306G/320R/332P/387I/390A/393G/534I/558W/681G/732W/749F/750D/775G/1080V/1148N/1248D/1377D/1439E, 63Q/171P/212A/260T/306G/320R/332P/387I/390A/393G/534I/558W/681G/732W/749F/750D/775G/806G/1148N/1248D/1352E/1377D/1439E, 63Q/171P/212A/260T/306G/320R/332P/387I/390A/393G/534I/558W/681G/732W/749F/750D/775G/1095G/1148N/1248D/1377D/1439E, 63Q/171P/212A/260T/306G/320R/332P/387I/390A/393G/534I/558W/681G/732W/749F/750D/775G/1093G/1148N/1248D/1377D/1439E, 63Q/171P/212A/260T/306G/320R/332P/387I/390A/393G/534I/558W/681G/732W/744A/749F/750D/775G/1148N/1248D/1377D/1439E, 63Q/171P/212A/260T/306G/320R/332P/387I/390A/393G/534I/543G/558W/681G/732W/749F/750D/775G/1148N/1248D/1377D/1439E, 63Q/171P/212A/260T/306G/320R/332P/387I/390A/393G/534I/558W/681G/732W/749F/750D/775G/1098G/1148N/1248D/1377D/1439E, 63Q/171P/212A/260T/281S/306G/320R/332P/387I/390A/393G/534I/558W/681G/732W/749F/750D/775G/1148N/1248D/1377D/1439E, 63Q/171P/212A/260T/306G/320R/332P/387I/390A/393G/534I/558W/681G/732W/749F/750D/756T/775G/1148N/1248D/1377D/1439E, 63Q/171P/212A/260T/306G/320R/332P/387I/390A/393G/534I/558W/681G/732W/749F/750D/775G/948S/1148N/1248D/1377D/1439E, 63Q/119V/171P/212A/260T/306G/320R/332P/387I/390A/393G/534I/558W/681G/732W/749F/750D/775G/1148N/1248D/1377D/1439E, 63Q/171P/212A/260T/306G/320R/332P/387I/390A/393G/534I/558W/681G/732W/749F/750D/775G/1116L/1148N/1248D/1377D/1439E, 63Q/171P/212A/260T/306G/320R/332P/387I/390A/393G/534I/558W/681G/732W/749F/750D/775G/785V/1148N/1248D/1377D/1439E, 63Q/171P/212A/260T/306G/320R/332P/387I/390A/393G/534I/558W/681G/732W/749F/750D/775G/1113F/1148N/1248D/1377D/1439E, 63Q/171P/212A/260T/306G/320R/332P/387I/390A/393G/534I/558W/681G/700A/732W/749F/750D/775G/1148N/1248D/1377D/1439E, 63Q/171P/212A/260T/306G/320R/332P/387I/390A/393G/534I/558W/681G/732W/749F/750D/775G/786D/1148N/1248D/1377D/1439E, 63Q/171P/175A/212A/260T/306G/320R/332P/387I/390A/393G/534I/558W/681G/732W/749F/750D/775G/1148N/1248D/1377D/1439E, 63Q/171P/212A/260T/306G/320R/332P/387I/390A/393G/534I/558W/681G/732W/749F/750D/775G/790M/1148N/1248D/1377D/1439E, 63Q/171P/212A/260T/306G/320R/332P/387I/390A/393G/534I/558W/667I/681G/732W/749F/750D/775G/1148N/1248D/1377D/1439E, 63Q/158G/171P/212A/260T/306G/320R/332P/387I/390A/393G/534I/558W/681G/732W/749F/750D/775G/1148N/1248D/1377D/1439E, 63Q/171P/212A/260T/306G/320R/332P/387I/390A/393G/534I/558W/580S/681G/732W/749F/750D/775G/1148N/1248D/1377D/1439E, 63Q/171P/212A/260T/306G/320R/332P/387I/390A/393G/534I/558W/681G/707H/732W/749F/750D/775G/1148N/1248D/1377D/1439E, 63Q/171P/212A/260T/306G/320R/332P/387I/390A/393G/534I/558W/681G/731I/732W/749F/750D/775G/1148N/1248D/1377D/1439E, 63Q/171P/212A/260T/306G/320R/332P/387I/390A/393G/534I/558W/681G/732W/749F/750D/775G/918M/1148N/1248D/1377D/1439E, 63Q/171P/212A/260T/306G/320R/332P/387I/390A/393G/534I/558W/681G/732W/749F/750D/775G/1100S/1148N/1248D/1377D/1439E, 63Q/171P/212A/260T/306G/320R/332P/387I/390A/393G/534I/558W/681G/732W/749F/750D/775G/1089S/1148N/1248D/1377D/1439E, 63Q/171P/212A/260T/306G/320R/332P/387I/390A/393G/534I/558W/681G/712Y/732W/749F/750D/775G/1148N/1248D/1377D/1439E, 63Q/171P/175H/212A/260T/306G/320R/332P/387I/390A/393G/534I/558W/681G/732W/749F/750D/775G/1148N/1248D/1377D/1439E, 63Q/171P/212A/260T/306G/320R/332P/387I/390A/393G/534I/558W/681G/732W/749F/750D/775G/1092G/1148N/1248D/1377D/1439E, 63Q/171P/212A/260T/306G/320R/332P/387I/390A/393G/534I/558W/681G/732W/749F/750D/775G/939E/1148N/1248D/1377D/1439E, 63Q/171P/212A/260T/306G/320R/332P/387I/390A/393G/534I/558W/681G/732W/749F/750D/775G/1104E/1148N/1248D/1377D/1439E, 63Q/171P/212A/260T/306G/320R/332P/387I/390A/393G/534I/558W/681G/732W/749F/750D/775G/800G/1148N/1248D/1377D/1439E, 63Q/171P/212A/260T/306G/320R/332P/387I/390A/393G/534I/558W/681G/732W/736F/749F/750D/775G/1148N/1248D/1377D/1439E, 63Q/171P/212A/260T/306G/320R/332P/387I/390A/393G/534I/543W/558W/681G/732W/749F/750D/775G/1148N/1248D/1377D/1439E, 63Q/171P/212A/260T/306G/320R/332P/387I/390A/393G/534I/558W/681G/732W/749F/750D/775G/1148N/1248D/1297A/1377D/1439E, 63Q/171P/212A/260T/306G/320R/332P/387I/390A/393G/534I/558W/681G/712L/732W/749F/750D/775G/1148N/1248D/1377D/1439E, 63Q/171P/212A/260T/306G/320R/332P/387I/390A/393G/534I/558W/681G/732W/749F/750D/775G/1127V/1148N/1248D/1377D/1439E, 63Q/171P/212A/260T/306G/320R/332P/387I/390A/393G/534I/558W/681G/732W/749F/750D/775G/939S/1148N/1248D/1377D/1439E, 63Q/171P/178M/212A/260T/306G/320R/332P/387I/390A/393G/534I/558W/681G/732W/749F/750D/775G/1148N/1248D/1377D/1439E, 63Q/171P/212A/260T/306G/320R/332P/387I/390A/393G/534I/558W/681G/731R/732W/749F/750D/775G/1148N/1248D/1377D/1439E, 63Q/171P/212A/260T/306G/320R/332P/387I/390A/393G/534I/558W/681G/686W/732W/749F/750D/775G/1148N/1248D/1377D/1439E, 63Q/171P/212A/260T/306G/320R/332P/387I/390A/393G/534I/543P/558W/681G/732W/749F/750D/775G/1148N/1248D/1377D/1439E, 63Q/171P/212A/260T/306G/320R/332P/387I/390A/393G/534I/558W/681G/732W/736G/749F/750D/775G/1148N/1248D/1377D/1439E, 63Q/171P/212A/260T/306G/320R/332P/387I/390A/393G/534I/558W/681G/732W/749F/750D/775G/1148N/1129P/1248D/1377D/1439E, 63Q/171P/212A/260T/306G/320R/332P/387I/390A/393G/534I/558W/681G/732W/749F/750D/775G/1138Q/1148N/1248D/1377D/1439E, 63Q/171P/212A/260T/306G/320R/332P/387I/390A/393G/534I/558W/681G/732W/749F/750D/775G/812V/1148N/1248D/1377D/1439E, 63Q/171P/212A/260T/306G/320R/332P/387I/390A/393G/534I/558W/681G/732W/749F/750D/775G/786D/821Q/1148N/1248D/1377D/1439E, 63Q/171P/212A/260T/306G/320R/332P/387I/390A/393G/534I/535C/558W/681G/732W/749F/750D/775G/1148N/1248D/1377D/1439E, 63Q/171P/212A/260T/306G/320R/332P/387I/390A/393G/534I/558W/680R/681G/732W/749F/750D/775G/1148N/1248D/1377D/1439E, 63Q/171P/212A/260T/306G/320R/332P/387I/390A/393G/534I/558W/681G/732W/749F/750D/775G/1116I/1148N/1248D/1377D/1439E, 63Q/171P/212A/260T/306G/320R/332P/387I/390A/393G/534I/558W/681G/732W/749F/750D/775G/1089F/1148N/1248D/1377D/1439E, 63Q/171P/212A/260T/304E/306G/320R/332P/387I/390A/393G/534I/558W/681G/732W/749F/750D/775G/1148N/1248D/1377D/1439E, 63Q/171P/178W/212A/260T/306G/320R/332P/387I/390A/393G/534I/558W/681G/732W/749F/750D/775G/1148N/1248D/1377D/1439E, 63Q/171P/212A/260T/306G/320R/332P/387I/390A/393G/534I/558W/681G/732W/749F/750D/775G/1104C/1148N/1248D/1377D/1439E, 63Q/171P/212A/260T/306G/320R/332P/387I/390A/393G/534I/558W/681G/732W/744R/749F/750D/775G/1148N/1248D/1377D/1439E, 63Q/171P/212A/260T/306G/320R/332P/387I/390A/393G/534I/558W/681G/732W/738R/749F/750D/775G/1148N/1248D/1377D/1439E, 63Q/171P/212A/260T/306G/320R/332P/387I/390A/393G/534I/558W/681G/732W/749F/750D/775G/1093 S/1148N/1248D/1377D/1439E, 63Q/171P/212A/260T/306G/320R/332P/387I/390A/393G/534I/558W/681G/712 S/732W/749F/750D/775G/1148N/1248D/1377D/1439E, 63Q/171P/187G/212A/260T/306G/320R/332P/387I/390A/393G/534I/558W/681G/732W/749F/750D/775G/1148N/1248D/1377D/1439E, 63Q/171P/212A/260T/306G/132P/320R/332P/387I/390A/393G/534I/558W/681G/732W/749F/750D/775G/1148N/1248D/1377D/1439E, 63Q/171P/212A/260T/306G/320R/332P/387I/390A/393G/534I/558W/681G/732W/749F/750D/775G/812I/1148N/1248D/1377D/1439E, 63Q/171P/212A/260T/306G/320R/332P/387I/390A/393G/534I/558W/681G/732W/749F/750D/775G/1080G/1148N/1248D/1377D/1439E, 63Q/171P/212A/260T/306G/320R/332P/387I/390A/393G/534I/558W/681G/732W/749F/750D/775G/800C/1148N/1248D/1377D/1439E, 63Q/171P/212A/260T/306G/320R/332P/387I/390A/393G/534I/558W/681G/732W/749F/750D/775G/1101G/1148N/1248D/1377D/1439E, 63Q/171P/212A/260T/306G/320R/332P/387I/390A/393G/534I/558W/681G/732W/749F/750D/775G/808K/1148N/1248D/1377D/1439E, 63Q/171P/212A/260T/306G/320R/332P/387I/390A/393G/534I/558W/681G/732W/749F/750D/775G/808S/1148N/1248D/1377D/1439E, 63Q/171P/212A/260T/306G/320R/332P/387I/390A/393G/534I/558W/681G/732W/749F/750D/775G/790A/1148N/1248D/1377D/1439E, 63Q/171P/212A/260T/306G/320R/332P/387I/390A/393G/534I/558W/681G/732W/749F/750D/775G/800V/1148N/1248D/1377D/1439E, 63Q/171P/212A/260T/306G/320R/332P/387I/390A/393G/534I/558W/680L/681G/732W/749F/750D/775G/1148N/1248D/1377D/1439E, 63Q/167R/171P/212A/260T/306G/320R/332P/387I/390A/393G/534I/558W/681G/732W/749F/750D/775G/1148N/1248D/1377D/1439E, 63Q/171P/212A/260T/306G/320R/332P/387I/390A/393G/534I/558W/681G/732W/749F/750D/752E/775G/1148N/1248D/1377D/1439E, 63Q/171P/212A/260T/306G/320R/332P/387I/390A/393G/534I/558W/681G/732W/749F/750D/775G/1093N/1148N/1248D/1377D/1439E, 63Q/119G/171P/212A/260T/306G/320R/332P/387I/390A/393G/534I/558W/681G/732W/749F/750D/775G/1148 N/1248D/1377D/1439E, 63Q/171P/212A/260T/306G/320R/332P/387I/390A/393G/534I/558W/681G/732W/739G/749F/750D/775G/1148N/1248D/1377D/1439E, 63Q/171P/212A/260T/306G/320R/332P/387I/390A/393G/534I/558W/681G/732W/749F/750D/775G/1116R/1148N/1248D/1377D/1439E, 63Q/171P/212A/260T/306G/320R/332P/387I/390A/393G/534I/558W/580Y/681G/732W/749F/750D/775G/1148N/1248D/1377D/1439E, 63Q/171P/212A/260T/304A/306G/320R/332P/387I/390A/393G/534I/558W/681G/732W/749F/750D/775G/1148N/1248D/1377D/1439E, 63Q/171P/212A/260T/306G/320R/332P/387I/390A/393G/534I/558W/681G/712Q/732W/749F/750D/775G/1148N/1248D/1377D/1439E, 63Q/171P/212A/260T/306G/320R/332P/387I/390A/393G/534I/558W/681G/732W/749F/750D/775G/806S/1148N/1248D/1377D/1439E, 63Q/171P/212A/260T/306G/320R/332P/387I/390A/393G/534I/558W/681G/712G/732W/749F/750D/775G/1148N/1248D/1377D/1439E, 63Q/171P/212A/260T/306G/320R/332P/387I/390A/393G/534I/558W/681G/732W/749F/750D/775G/790G/1148N/1248D/1377D/1439E, 63Q/171P/212A/260T/306G/320R/332P/387I/390A/393G/534I/558W/681G/732W/749F/750D/775G/1108V/1148N/1248D/1377D/1439E, 63Q/171P/212A/260T/306G/320R/332P/387I/390A/393G/534I/558W/681G/731L/732W/749F/750D/775G/1148N/1248D/1377D/1439E, 63Q/171P/212A/260T/306G/320R/332P/387I/390A/393G/534I/558W/681G/732W/749F/750D/775G/1084N/1148N/1248D/1377D/1439E, 63Q/171P/212A/260T/306G/320R/332P/387I/390A/393G/534I/558W/681G/732W/749F/750D/775G/1127L/1148N/1248D/1377D/1439E, 63Q/171P/212A/260T/306G/308P/320R/332P/387I/390A/393G/534I/558W/681G/732W/749F/750D/775G/1148N/1248D/1377D/1439E, 63Q/171P/212A/260T/306G/320R/332P/387I/390A/393G/534I/558W/681G/732W/749F/750D/775G/1076S/1148N/1248D/1377D/1439E, 63Q/171P/212A/260T/306G/320R/332P/387I/390A/393G/534I/558W/681G/732W/749F/750D/775G/1093P/1148N/1248D/1377D/1439E, 63Q/171P/212A/260T/306G/320R/332P/387I/390A/393G/534I/558W/681G/732W/749F/750D/775G/1093Y/1148N/1248D/1377D/1439E, 63Q/171P/212A/260T/306G/320R/332P/387I/390A/393G/534I/558W/681G/732W/739R/749F/750D/775G/1148N/1248D/1377D/1439E, 63Q/171P/212A/260T/306G/320R/332P/387I/390A/393G/534I/558W/681G/732W/739P/749F/750D/775G/1148N/1248D/1377D/1439E, 63Q/171P/212A/260T/306G/310V/320R/332P/387I/390A/393G/534I/558W/681G/732W/749F/750D/775G/1148N/1248D/1377D/1439E, 63Q/171P/212A/260T/306G/320R/332P/387I/390A/393G/534I/558W/681G/712T/732W/749F/750D/775G/1148N/1248D/1377D/1439E, 63Q/167H/171P/212A/260T/306G/320R/332P/387I/390A/393G/534I/558W/681G/732W/749F/750D/775G/1148N/1248D/1377D/1439E, 63Q/171P/212A/260T/306G/320R/332P/387I/390A/393G/534I/558W/681G/732W/749F/750D/775G/1093R/1148N/1248D/1377D/1439E, 63Q/171P/212A/260T/306G/320R/332P/387I/390A/393G/534I/536P/555E/558W/619E/681G/749F/750D/775G/799S/805Q/1148N/1248D/1377D/1439E, 63Q/171P/212A/260T/306G/320R/332P/387I/390A/393G/534I/555E/558W/619E/681G/732W/749F/750D/775G/801G/1148N/1248D/1377D/1439E, 63Q/171P/212A/260T/306G/320R/332P/387I/390A/393G/534I/555E/558W/619E/681G/732W/749F/750D/775G/799S/1148N/1248D/1377D/1439E, 63Q/171P/212A/260T/306G/320R/332P/387I/390A/393G/534I/558W/619E/681G/732W/749F/750D/775G/799S/801G/805Q/1148N/1248D/1377D/1439E, 63Q/171P/212A/260T/306G/320R/332P/387I/390A/393G/534I/536P/555E/558W/619E/681G/749F/750D/775G/799S/1148N/1248D/1377D/1439E, 63Q/171P/212A/260T/306G/320R/332P/387I/390A/393G/534I/558W/681G/732W/749F/750D/775G/799S/805Q/1148N/1248D/1377D/1439E, 63Q/171P/212A/260T/306G/320R/332P/387I/390A/393G/534I/558W/681G/732W/749F/750D/775G/801G/1148N/1248D/1377D/1439E, 63Q/171P/212A/246D/260T/306G/320R/332P/387I/390A/393G/534I/558W/619E/681G/732W/749F/750D/775G/799S/801G/1148N/1248D/1377D/1439E, 63Q/171P/212A/260T/306G/320R/332P/387I/390A/393G/534I/536P/555E/558W/619E/681G/732W/749F/75 OD/775G/1148N/1248D/1377D/1439E, 63Q/171P/212A/260T/306G/320R/332P/387I/390A/393G/534I/558W/619E/681G/732W/749F/750D/775G/839K/1148N/1248D/1377D/1439E, 63Q/171P/212A/260T/306G/320R/332P/387I/390A/393G/534I/558W/619E/681G/732W/749F/750D/775G/799S/805Q/1148N/1248D/1377D/1439E, 63Q/171P/212A/260T/306G/320R/332P/387I/390A/393G/534I/536P/558W/681G/732W/749F/750D/775G/801G/805Q/1148N/1248D/1377D/1439E, 63Q/171P/212A/260T/306G/320R/332P/387I/390A/393G/534I/555E/558W/619E/681G/732W/749F/750D/775G/799S/805Q/1148N/1248D/1377D/1439E, 63Q/171P/212A/260T/306G/320R/332P/387I/390A/393G/534I/558W/619E/681G/732W/749F/750D/775G/801G/805Q/1148N/1248D/1377D/1439E, 63Q/171P/212A/260T/306G/320R/332P/387I/390A/393G/534I/555E/558W/619E/681G/732W/749F/750D/775G/1148N/1248D/1377D/1439E, 63Q/171P/212A/260T/306G/320R/332P/387I/390A/393G/534I/536P/558W/619E/681G/732W/749F/750D/775G/801G/1148N/1248D/1377D/1439E, 63Q/171P/212A/260T/306G/320R/332P/387I/390A/393G/534I/558W/681G/732W/749F/750D/775G/801G/805Q/1148N/1248D/1377D/1439E, 63Q/171P/212A/260T/306G/320R/332P/387I/390A/393G/534I/536P/558W/619E/681G/732W/749F/750D/775G/799S/805Q/1148N/1248D/1377D/1439E, 63Q/171P/212A/260T/306G/320R/332P/387I/390A/393G/534I/555E/558W/619E/681G/749F/750D/775G/799S/801G/805Q/1148N/1248D/1377D/1439E, 63Q/171P/212A/260T/306G/320R/332P/387I/390A/393G/534I/558W/681G/732W/749F/750D/775G/1148N/1248D/1281H/1377D/1439E, 63Q/171P/212A/260T/306G/320R/332P/387I/390A/393G/534I/558W/681G/732W/750D/775G/1148N/1248 D/1377D/1439E, 63Q/171P/212A/260T/306G/320R/332P/387I/390A/393G/534I/558W/614H/681G/732W/749F/750D/775G/1148N/1248D/1377D/1439E, 63Q/171P/212A/260T/306G/320R/332P/387I/390A/393G/534I/558W/616D/681G/732W/749F/750D/775G/1148N/1248D/1377D/1439E, 63Q/171P/212A/260T/306G/320R/332P/387I/390A/393G/534I/558W/621K/681G/732W/749F/750D/775G/1148N/1248D/1377D/1439E, 63Q/171P/212A/260T/306G/320R/332P/387I/390A/393G/534I/558W/681G/732W/749F/750D/775G/1050E/1148N/1248D/1377D/1439E, 63Q/171P/212A/260T/306G/320R/332P/387I/390A/393G/534I/558W/610H/681G/732W/749F/750D/775G/1148N/1248D/1377D/1439E, 63Q/171P/212A/260T/306G/320R/332P/387I/390A/393G/534I/558W/611D/681G/732W/749F/750D/775G/1148N/1248D/1377D/1439E, 63Q/171P/212A/260T/306G/320R/332P/387I/390A/393G/534I/558W/681G/732W/749F/750D/775G/1042T/1148N/1248D/1377D/1439E, 63Q/171P/212A/260T/306G/320R/332P/387I/390A/393G/534I/558W/616Q/681G/732W/749F/750D/775G/1148N/1248D/1377D/1439E, 63Q/171P/212A/260T/306G/320R/332P/387I/390A/393G/534I/558W/614N/681G/732W/749F/750D/775G/1148N/1248D/1377D/1439E, 63Q/171P/212A/260T/306G/320R/332P/370A/387I/390A/393G/534I/558W/614A/681G/732W/749F/750D/775G/1148N/1248D/1377D/1439E, 63Q/171P/212A/260T/306G/320R/332P/387I/390A/393G/534I/558W/681G/732W/749F/750D/775G/1148N/1156D/1248D/1377D/1439E, 63Q/171P/212A/260T/306G/320R/332P/387I/390A/393G/534I/558W/681G/732W/749F/750D/775G/1148N/1210V/1248D/1377D/1439E, 63Q/171P/212A/260T/306G/320R/332P/387I/390A/393G/534I/558W/614T/681G/732W/749F/750D/775G/1148N/1248D/1377D/1439E, 63Q/171P/212A/260T/306G/320R/332P/387I/390A/393G/534I/558W/681G/732W/749F/750D/775G/1148N/1158N/1248D/1377D/1439E, 63Q/171P/212A/260T/306G/320R/332P/387I/390A/393G/534I/558W/681G/732W/749F/750D/775G/1148N/1248D/1281S/1377D/1439E, 63Q/171P/212A/260T/306G/320R/332P/387I/390A/393G/534I/558W/618E/681G/732W/749F/750D/775G/1148N/1248D/1377D/1439E, 63Q/171P/212A/260T/306G/320R/332P/387I/390A/393G/534I/555E/558W/681N/732W/737K/749F/750D/775G/799Q/1148N/1248D/1377D/1439E, 63Q/171P/212A/260T/306G/320R/332P/387I/390A/393G/534I/536P/558W/619E/681G/732W/749F/750D/775G/809G/1109G/1148N/1248D/1377D/1439E, 63Q/171P/212A/260T/306G/320R/332P/387I/390A/393G/534I/536K/555G/558W/619E/681G/732W/737K/749F/750D/775G/799Q/809G/839K/916T/921R/1148N/1248D/1377D/1439E, 63Q/171P/212A/260T/306G/320R/332P/387I/390A/393G/534I/558W/681G/732W/749F/750D/775G/916T/1109G/1148N/1248D/1377D/1439E, 63Q/171P/212A/260T/306G/320R/332P/387I/390A/393G/534I/536R/558W/619E/681N/732W/749F/750D/775G/799Q/916T/1109G/1148N/1248D/1377D/1439E, 63Q/171P/212A/260T/306G/320R/332P/387I/390A/393G/534I/555G/558W/619E/681N/732W/749F/750D/775G/807R/1109G/1148N/1248D/1377D/1439E, 63Q/171P/212A/260T/306G/320R/332P/387I/390A/393G/534I/555E/558W/619E/681N/749F/750D/775G/799Q/1148N/1248D/1377D/1439E, 63Q/171P/212A/260T/306G/320R/332P/387I/390A/393G/534I/558W/619E/681N/732W/749F/750D/775G/799S/916T/1109G/1148N/1248D/1377D/1439E, 63Q/171P/212A/260T/306G/320R/332P/387I/390A/393G/534I/558W/619E/681N/732W/749F/750D/775G/1148N/1248D/1377D/1439E, 63Q/171P/212A/260T/306G/320R/332P/387I/390A/393G/534I/555G/558W/681G/732W/749F/750D/775G/921R/1148N/1248D/1377D/1439E, 63Q/171P/212A/260T/306G/320R/332P/387I/390A/393G/534I/558W/619E/681N/732W/749F/750D/775G/916T/1148N/1248D/1377D/1439E, 63Q/171P/212A/260T/306G/320R/332P/387I/390A/393G/534I/555G/558W/619E/681N/732W/749F/750D/775G/799S/1148N/1248D/1377D/1439E, 63Q/171P/212A/260T/306G/320R/332P/387I/390A/393G/534I/555E/558W/681G/732W/749F/750D/775G/1148N/1248D/1377D/1439E, 63Q/171P/212A/260T/306G/320R/332P/387I/390A/393G/534I/558W/681G/749F/750D/775G/1148N/1248D/1377D/1439E, 63Q/171P/212A/260T/306G/320R/332P/387I/390A/393G/534I/558W/619E/681G/732W/749F/750D/775G/799S/1148N/1248D/1377D/1439E, 63Q/171P/212A/260T/306G/320R/332P/387I/390A/393G/534I/558W/619E/681G/732W/749F/750D/775G/1148N/1248D/1377D/1439E, 63Q/171P/212A/260T/306G/320R/332P/387I/390A/393G/534I/558W/681N/732W/749F/750D/775G/1148N/1248D/1377D/1439E, 63Q/171P/212A/260T/306G/320R/332P/387I/390A/393G/534I/555G/558W/681G/732W/749F/750D/775G/916T/1148N/1248D/1377D/1439E, 63Q/171P/212A/260T/306G/320R/332P/387I/390A/393G/534I/558W/681G/732W/749F/750D/775G/839K/1148N/1248D/1377D/1439E, or 63Q/171P/212A/260T/306G/320R/332P/387I/390A/393G/534I/558W/681G/732W/749F/750D/775G/799Q/1148N/1248D/1377D/1439E, wherein the amino acid positions are relative to the reference sequence corresponding to SEQ ID NO: 2.

In some embodiments, the amino acid sequence of the recombinant factor VIII polypeptide comprises at least a substitution or substitution set at amino acid position(s) 63/167/171/212/260/306/318/320/332/387/390/393/534/558/681/732/738/749/750/775/1148/1248/1377/1439, 63/167/171/212/260/306/318/320/332/387/390/393/534/558/681/732/749/750/775/1076/1093/1148/1248/1377/1439, 63/167/171/212/260/306/318/320/332/387/390/393/534/558/681/732/749/750/752/775/800/805/808/809/1116/1148/1248/1377/1439, 63/167/171/212/260/306/318/320/332/387/390/393/534/558/681/732/738/749/750/775/800/809/1076/1148/1248/1377/1439, 63/167/171/212/260/306/320/332/387/390/393/534/558/681/732/738/749/750/775/1148/1248/1377/1439, 63/167/171/212/260/306/320/332/387/390/393/534/558/681/732/738/749/750/775/800/805/808/809/1148/1248/1377/1439, 63/167/171/212/260/306/320/332/387/390/393/534/558/681/700/732/738/749/750/775/805/808/809/1076/1116/1148/1248/1377/1439, 63/167/171/212/260/306/320/332/387/390/393/534/558/681/700/732/738/749/750/775/1148/1248/1377/1439, 63/167/171/212/260/306/320/332/387/390/393/534/558/681/700/732/738/749/750/775/1076/1148/1248/1377/1439, 63/167/171/212/260/306/320/332/387/390/393/534/558/681/700/732/739/749/750/775/1076/1148/1248/1377/1439, 63/167/171/212/260/306/318/320/332/387/390/393/534/558/681/732/749/750/775/800/808/809/1148/1248/1377/1439, 63/167/171/L178/212/260/306/318/320/332/387/390/393/534/558/681/700/732/738/749/750/752/775/1093/1148/1248/1377/1439, 63/167/171/212/260/306/318/320/332/387/390/393/534/558/681/700/732/738/749/750/775/1148/1248/1377/1439, 63/167/171/212/260/306/318/320/332/387/390/393/534/558/681/700/732/739/749/750/775/1093/1148/1248/1377/1439, 63/167/171/212/260/306/318/320/332/387/390/393/534/558/681/700/732/749/750/752/775/1093/1148/1248/1377/1439, 63/167/171/212/260/306/318/320/332/387/390/393/534/558/681/700/732/749/750/752/775/805/808/809/1093/1116/1148/1248/1377/1439, 63/171/212/260/306/318/320/332/387/390/393/534/558/681/732/738/749/750/775/808/809/1148/1248/1377/1439, 63/171/212/260/306/318/320/332/387/390/393/534/558/681/732/738/749/750/775/1076/1148/1248/1377/1439, 63/171/212/260/306/318/320/332/387/390/393/534/558/681/732/749/750/775/1148/1248/1377/1439, 63/171/212/260/306/318/320/332/387/390/393/534/558/681/732/749/750/775/808/809/1148/1248/1377/1439, 63/171/212/260/306/318/320/332/387/390/393/534/558/681/732/749/750/775/1076/1148/1248/1377/1439, 63/171/212/260/306/318/320/332/387/390/393/534/558/681/732/739/749/750/775/1148/1248/1377/1439, 63/171/212/260/306/318/320/332/387/390/393/534/558/681/732/739/749/750/775/809/1148/1248/1377/1439, 63/171/212/260/306/318/320/332/387/390/393/534/558/681/732/739/749/750/775/809/1093/1116/1148/1248/1377/1439, 63/171/212/260/306/318/320/332/387/390/393/534/558/681/732/739/749/750/775/1093/1148/1248/1377/1439, 63/171/212/260/306/318/320/332/387/390/393/534/558/681/732/739/749/750/752/775/1093/1148/1248/1377/1439, 63/171/212/260/306/318/320/332/387/390/393/534/558/681/732/739/749/750/775/799/800/1093/1148/1248/1377/1439, 63/171/212/260/306/318/320/332/387/390/393/534/558/681/732/739/749/750/775/1116/1148/1248/1377/1439, 63/171/212/260/306/318/320/332/387/390/393/534/558/681/732/749/750/775/800/1148/1248/1377/1439, 63/171/212/260/306/318/320/332/387/390/393/534/558/681/732/749/750/775/1093/1148/1248/1377/1439, 63/171/212/260/306/318/320/332/387/390/393/534/558/681/732/749/750/752/775/1148/1248/1377/1439, 63/171/212/260/306/318/320/332/387/390/393/534/558/681/732/749/750/752/775/1093/1148/1248/1377/1439, 63/171/212/260/306/318/320/332/387/390/393/534/558/681/732/749/750/752/775/799/1148/1248/1377/1439, 63/171/212/260/306/318/320/332/387/390/393/534/558/681/732/749/750/775/799/809/1093/1148/1248/1377/1439, 63/171/212/260/306/318/320/332/387/390/393/534/558/681/732/749/750/775/799/1093/1148/1248/1377/1439, 63/171/212/260/306/318/320/332/387/390/393/534/558/681/732/738/739/749/750/775/1148/1248/1377/1439, 63/171/212/260/306/318/320/332/387/390/393/534/558/681/732/738/739/749/750/775/800/809/1148/1248/1377/1439, 63/171/212/260/306/318/320/332/387/390/393/534/558/681/732/738/749/750/775/800/1148/1248/1377/1439, 63/171/212/260/306/318/320/332/387/390/393/534/558/681/732/738/749/750/775/800/805/809/1148/1248/1377/1439, 63/171/212/260/306/318/320/332/387/390/393/534/558/681/732/738/749/750/775/809/1148/1248/1377/1439, 63/171/212/260/306/318/320/332/387/390/393/534/558/681/732/738/749/750/775/809/1093/1148/1248/1377/1439, 63/171/212/260/306/318/320/332/387/390/393/534/558/681/732/738/749/750/775/809/1093/1116/1148/1248/1377/1439, 63/171/212/260/306/318/320/332/387/390/393/534/558/681/732/738/749/750/775/809/1116/1148/1248/1377/1439, 63/171/212/260/306/320/332/387/390/393/534/558/681/732/738/749/750/775/1148/1248/1377/1439, 63/171/212/260/306/320/332/387/390/393/534/558/681/732/738/749/750/775/1076/1148/1248/1377/1439, 63/171/212/260/306/320/332/387/390/393/534/558/681/732/738/749/750/775/1076/1116/1148/1248/1377/1439, 63/171/212/260/306/320/332/387/390/393/534/558/681/732/738/739/749/750/775/805/808/809/1076/1148/1248/1377/1439, 63/171/212/260/306/320/332/387/390/393/499/534/558/681/700/732/749/750/775/809/1148/1248/1377/1439, 63/171/212/260/306/320/332/387/390/393/534/558/681/732/738/749/750/775/1093/1148/1248/1377/1439, 63/171/212/260/306/320/332/387/390/393/534/558/681/732/738/749/750/752/775/1093/1148/1248/1377/1439, 63/171/212/260/306/320/332/387/390/393/534/558/681/732/738/749/750/775/1116/1148/1248/1377/1439, 63/171/212/260/306/320/332/387/390/393/534/543P/558/681/732/738/739/749/750/775/1148/1248/1377/1439, 63/171/212/260/306/320/332/387/390/393/534/543/558/681/732/738/749/750/752/775/1116/1148/1248/1377/1439, 63/171/212/260/306/320/332/387/390/393/534/543/558/681/732/738/749/750/775/1093/1148/1248/1377/1439, 63/171/212/260/306/320/332/387/390/393/534/558/681/700/732/749/750/775/1093/1148/1248/1377/1414/1439, 63/171/212/260/306/318/320/332/387/390/393/534/558/681/732/738/749/750/775/1148/1248/1377/1439, 63/171/212/260/306/318/320/332/387/390/393/534/558/681/732/749/750/775/1148/1248/1377/1439, 63/171/212/260/306/318/320/332/387/390/393/534/558/681/732/739/749/750/752/775/799/800/809/1116/1148/1248/1339/1377/1439, 63/171/212/260/306/318/320/332/387/390/393/534/558/681/732/749/750/775/800/1148/1248/1377/1439, 63/171/212/260/306/318/320/332/387/390/393/534/558/681/732/738/749/750/752/775/1093/1148/1248/1377/1439, 63/171/212/260/306/318/320/332/387/390/393/534/558/681/700/732/738/749/750/775/1148/1248/1377/1439, 63/171/212/260/306/318/320/332/387/390/393/534/558/681/700/732/739/749/750/775/1093/1116/1148/1248/1377/1439, 63/171/212/260/306/318/320/332/387/390/393/534/558/681/700/732/738/749/750/775/809/1148/1248/1377/1439, 63/171/212/260/306/318/320/332/387/390/393/534/558/681/700/732/738/749/750/775/1093/1148/1248/1377/1439, 63/171/212/260/306/318/320/332/387/390/393/534/558/681/700/732/738/749/750/752/775/808/809/1093/1116/1148/1248/1377/1439, 63/171/212/260/306/318/320/332/387/390/393/534/558/681/700/732/738/749/750/752/775/1093/1148/1248/1377/1439, 63/171/212/260/306/318/320/332/387/390/393/534/558/681/700/732/738/749/750/775/844/1148/1248/1377/1439, 63/171/212/260/306/318/320/332/387/390/393/534/558/681/700/732/738/749/750/775/805/809/1148/1248/1377/1439, 63/171/212/260/306/318/320/332/387/390/393/534/558/681/732/738/749/750/775/1093/1148/1248/1377/1439, 63/171/212/260/306/318/320/332/387/390/393/534/558/681/732/738/749/750/775/1093/1116/1148/1248/1377/1439, 63/171/212/260/306/318/320/332/387/390/393/534/558/681/732/738/749/750/752/775/1148/1248/1377/1439, 63/171/212/260/306/318/320/332/387/390/393/534/558/681/732/738/749/750/752/775/1076/1148/1248/1377/1439, 63/171/212/260/306/318/320/332/387/390/393/534/558/681/732/738/749/750/752/775/800/809/1148/1248/1377/1439, 63/171/212/260/306/318/320/332/387/390/393/534/558/681/732/738/749/750/752/775/800/1093/1148/1248/1377/1439, 63/171/212/260/306/318/320/332/387/390/393/534/558/681/732/738/749/750/752/775/809/1148/1248/1377/1439, 63/171/212/260/306/318/320/332/387/390/393/534/558/681/732/738/749/750/752/775/866/1148/1248/1377/1439, 63/171/212/260/306/318/320/332/387/390/393/534/558/681/732/738/749/750/752/775/1116/1148/1248/1377/1439, 63/171/178/212/260/306/320/332/387/390/393/534/558/681/732/738/739/749/750/752/775/809/1076/1116/1148/1248/1377/1439, 63/171/178/212/260/306/320/332/387/390/393/534/558/681/732/738/749/750/775/800/809/1148/1248/1377/1439, 63/171/178/212/260/306/320/332/387/390/393/534/558/681/732/738/749/750/775/800/808/809/1076/1148/1248/1377/1439, 63/171/178/212/260/306/318/320/332/387/390/393/534/558/681/732/738/749/750/775/1148/1248/1377/1439, 63/171/178/212/260/306/318/320/332/387/390/393/534/558/681/732/749/750/775/1116/1148/1248/1377/1439, 63/171/178/212/260/306/318/320/332/387/390/393/534/558/681/732/738/749/750/752/775/809/1148/1248/1377/1439, 63/171/178/212/260/306/318/320/332/387/390/393/534/558/681/700/732/738/749/750/775/800/805/808/809/1148/1248/1377/1439, 63/171/178/212/260/306/318/320/332/387/390/393/534/558/681/732/738/749/750/775/805/809/866/1148/1248/1377/1439, 63/171/212/260/306/318/320/332/387/390/393/534/558/681/732/738/749/750/775/1116/1148/1248/1377/1439, 63/171/212/260/306/318/320/332/387/390/393/534/543/558/681/732/738/749/750/775/1148/1248/1377/1439, 63/171/212/260/306/318/320/332/387/390/393/534/543/558/681/732/739/749/750/775/809/1148/1248/1377/1439, 63/171/212/260/306/318/320/332/387/390/393/534/543/558/681/732/749/750/775/809/1093/1148/1248/1377/1439, 63/171/212/260/306/318/320/332/387/390/393/534/543/558/681/732/738/739/749/750/775/1093/1148/1248/1377/1439, 63/171/212/260/306/318/320/332/387/390/393/534/543/558/681/732/738/749/750/775/800/1093/1116/1148/1248/1377/1439, 63/171/212/260/306/318/320/332/387/390/393/534/543/558/681/732/738/749/750/775/809/1093/1148/1248/1377/1439, 63/171/212/260/306/318/320/332/387/390/393/534/543/558/681/732/738/749/750/775/809/1093/1116/1148/1248/1377/1439, 63/171/212/260/306/318/320/332/387/390/393/534/543/558/681/732/739/749/750/752/775/809/1093/1148/1248/1377/1439, 63/171/212/260/306/318/320/332/387/390/393/534/543/558/681/732/739/749/750/752/775/1093/1148/1248/1377/1439, 63/171/212/260/306/318/320/332/387/390/393/534/543/558/681/732/738/739/749/750/752/775/800/805/809/1093/1148/1248/1377/1439, 63/171/212/260/306/318/320/332/387/390/393/534/543/558/681/700/732/739/749/750/775/800/1093/1116/1148/1248/1377/1439, 63/171/212/260/306/318/320/332/387/390/393/534/543/558/681/700/732/739/749/750/775/805/808/809/1148/1248/1377/1439, 63/171/212/260/306/318/320/332/387/390/393/534/543/558/681/700/732/738/749/750/752/775/1148/1248/1377/1439, 63/171/212/260/306/318/320/332/387/390/393/534/543/558/681/700/732/738/749/750/752/775/809/1093/1148/1248/1377/1439, 63/171/212/260/306/318/320/332/387/390/393/534/543/558/681/732/738/749/750/775/1093/1148/1248/1377/1439, 63/171/212/260/306/318/320/332/387/390/393/534/543/558/681/700/732/738/749/750/775/1148/1248/1377/1439, 63/171/212/260/306/318/320/332/387/390/393/534/543/558/681/700/732/739/749/750/752/775/800/1093/1116/1148/1248/1377/1439, 63/171/212/260/306/318/320/332/387/390/393/534/543/558/681/700/732/738/749/750/775/809/1148/1248/1377/1439, 63/171/212/260/306/318/320/332/387/390/393/534/543/558/681/700/732/738/749/750/775/1093/1148/1248/1377/1439, 63/171/212/260/306/318/320/332/387/390/393/534/543/558/681/700/732/738/749/750/752/775/800/1093/1148/1248/1377/1439, 63/171/212/260/306/318/320/332/387/390/393/534/543/558/681/700/732/738/749/750/752/775/809/1116/1148/1248/1377/1439, 63/171/212/260/306/318/320/332/387/390/393/534/543/558/681/700/732/738/749/750/752/775/1093/1148/1248/1377/1439, 63/171/212/260/306/318/320/332/387/390/393/534/558/681/700/732/739/749/750/775/800/1093/1148/1248/1377/1439, 63/171/212/260/306/318/320/332/387/390/393/534/558/681/700/732/739/749/750/775/799/1093/1148/1248/1377/1439, 63/171/212/260/306/318/320/332/387/390/393/534/558/681/700/732/749/750/775/800/1093/1148/1248/1377/1439, 63/171/212/260/306/318/320/332/387/390/393/534/558/681/700/732/749/750/775/1116/1148/1248/1377/1439, 63/171/212/260/306/318/320/332/387/390/393/534/558/681/700/732/738/749/750/775/809/1093/1148/1248/1377/1439, 63/171/212/260/306/318/320/332/387/390/393/534/558/681/700/732/738/749/750/775/1093/1116/1148/1248/1377/1439, 63/171/212/260/306/318/320/332/387/390/393/534/558/681/700/732/738/749/750/752/775/1148/1248/1377/1439, 63/171/212/260/306/318/320/332/387/390/393/534/558/681/700/732/738/749/750/752/775/809/1093/1148/1248/1377/1439, 63/171/212/260/306/318/320/332/387/390/393/534/558/681/700/732/738/749/750/752/775/799/1116/1148/1248/1377/1439, 63/171/212/260/306/318/320/332/387/390/393/534/558/681/700/732/738/749/750/752/775/805/809/1093/1148/1248/1377/1439, 63/171/212/260/306/318/320/332/387/390/393/534/558/681/700/732/738/749/750/775/799/800/1148/1248/1377/1439, 63/171/212/260/306/318/320/332/387/390/393/534/558/681/700/732/738/749/750/775/1116/1148/1248/1377/1439, 63/171/212/260/306/318/320/332/387/390/393/534/558/681/700/732/738/749/750/775/805/808/809/1076/1148/1248/1377/1439, 63/171/212/260/306/318/320/332/387/390/393/514/534/543/558/681/732/738/749/750/775/805/808/809/1148/1248/1377/1439, 63/171/212/260/306/318/320/332/387/390/393/534/558/681/732/738/749/750/775/805/808/809/1148/1248/1377/1439, or 63/171/212/260/306/318/320/332/387/390/393/534/558/681/732/738/749/750/775/805/809/1116/1148/1248/1377/1439, wherein the amino acid positions are relative to the reference sequence corresponding to SEQ ID NO: 2.

In some embodiments, the amino acid sequence of the recombinant factor VIII polypeptide comprises at least a substitution or substitution set 63Q/167G/171P/212A/260T/306G/318G/320R/332P/387I/390A/393G/534I/558W/681G/732W/738E/749F/750D/775G/1148N/1248D/1377D/1439E, 63Q/167G/171P/212A/260T/306G/318G/320R/332P/387I/390A/393G/534I/558W/681G/732W/749F/750D/775G/1076S/1093G/1148N/1248D/1377D/1439E, 63Q/167G/171P/212A/260T/306G/318G/320R/332P/387I/390A/393G/534I/558W/681G/732W/749F/750D/752E/775G/800V/805Q/808S/809G/1116I/1148N/1248D/1377D/1439E, 63Q/167G/171P/212A/260T/306G/318G/320R/332P/387I/390A/393G/534I/558W/681G/732W/738E/749F/750D/775G/800V/809G/1076S/1148N/1248D/1377D/1439E, 63Q/167G/171P/212A/260T/306G/320R/332P/387I/390A/393G/534I/558W/681G/732W/738E/749F/750D/775G/1148N/1248D/1377D/1439E, 63Q/167G/171P/212A/260T/306G/320R/332P/387I/390A/393G/534I/558W/681G/732W/738E/749F/750D/775G/800V/805Q/808S/809G/1148N/1248D/1377D/1439E, 63Q/167G/171P/212A/260T/306G/320R/332P/387I/390A/393G/534I/558W/681N/732W/738E/749F/750D/775G/1148N/1248D/1377D/1439E, 63Q/167G/171P/212A/260T/306G/320R/332P/387I/390A/393G/534I/558W/681N/700E/732W/738E/749F/75 OD/775G/805Q/808S/809G/1076S/1116I/1148N/1248D/1377D/1439E, 63Q/167G/171P/212A/260T/306G/320R/332P/387I/390A/393G/534I/558W/681G/700E/732W/738E/749F/75 OD/775G/1148N/1248D/1377D/1439E, 63Q/167G/171P/212A/260T/306G/320R/332P/387I/390A/393G/534I/558W/681G/700E/732W/738E/749F/75 OD/775G/1076S/1148N/1248D/1377D/1439E, 63Q/167G/171P/212A/260T/306G/320R/332P/387I/390A/393G/534I/558W/681G/700E/732W/739G/749F/750D/775G/1076S/1148N/1248D/1377D/1439E, 63Q/167G/171P/212A/260T/306G/318G/320R/332P/387I/390A/393G/534I/558W/681N/732W/749F/750D/775G/800V/808S/809G/1148N/1248D/1377D/1439E, 63Q/167G/171P/178Q/212A/260T/306G/318G/320R/332P/387I/390A/393G/534I/558W/681G/700E/732W/738E/749F/750D/752E/775G/1093G/1148N/1248D/1377D/1439E, 63Q/167G/171P/212A/260T/306G/318G/320R/332P/387I/390A/393G/534I/558W/681G/700E/732W/738E/749F/750D/775G/1148N/1248D/1377D/1439E, 63Q/167G/171P/212A/260T/306G/318G/320R/332P/387I/390A/393G/534I/558W/681G/700E/732W/739G/749F/750D/775G/1093G/1148N/1248D/1377D/1439E, 63Q/167G/171P/212A/260T/306G/318G/320R/332P/387I/390A/393G/534I/558W/681G/700E/732W/749F/750D/752E/775G/1093G/1148N/1248D/1377D/1439E, 63Q/167G/171P/212A/260T/306G/318G/320R/332P/387I/390A/393G/534I/558W/681G/700E/732W/749F/750D/752E/775G/805Q/808S/809G/1093A/1116I/1148N/1248D/1377D/1439E, 63Q/171P/212A/260T/306G/318G/320R/332P/387I/390A/393G/534I/558W/681G/732W/738E/749F/750D/775G/808S/809G/1148N/1248D/1377D/1439E, 63Q/171P/212A/260T/306G/318G/320R/332P/387I/390A/393G/534I/558W/681G/732W/738E/749F/750D/775G/1076S/1148N/1248D/1377D/1439E, 63Q/171P/212A/260T/306G/318G/320R/332P/387I/390A/393G/534I/558W/681G/732W/749F/750D/775G/1148N/1248D/1377D/1439E, 63Q/171P/212A/260T/306G/318G/320R/332P/387I/390A/393G/534I/558W/681G/732W/749F/750D/775G/808S/809G/1148N/1248D/1377D/1439E, 63Q/171P/212A/260T/306G/318G/320R/332P/387I/390A/393G/534I/558W/681G/732W/749F/750D/775G/1076S/1148N/1248D/1377D/1439E, 63Q/171P/212A/260T/306G/318G/320R/332P/387I/390A/393G/534I/558W/681G/732W/739G/749F/750D/775G/1148N/1248D/1377D/1439E, 63Q/171P/212A/260T/306G/318G/320R/332P/387I/390A/393G/534I/558W/681G/732W/739G/749F/750D/775G/809G/1148N/1248D/1377D/1439E, 63Q/171P/212A/260T/306G/318G/320R/332P/387I/390A/393G/534I/558W/681G/732W/739G/749F/750D/775G/809G/1093G/1116I/1148N/1248D/1377D/1439E, 63Q/171P/212A/260T/306G/318G/320R/332P/387I/390A/393G/534I/558W/681G/732W/739G/749F/750D/775G/1093G/1148N/1248D/1377D/1439E, 63Q/171P/212A/260T/306G/318G/320R/332P/387I/390A/393G/534I/558W/681G/732W/739G/749F/750D/752E/775G/1093G/1148N/1248D/1377D/1439E, 63Q/171P/212A/260T/306G/318G/320R/332P/387I/390A/393G/534I/558W/681G/732W/739G/749F/750D/775G/799S/800V/1093G/1148N/1248D/1377D/1439E, 63Q/171P/212A/260T/306G/318G/320R/332P/387I/390A/393G/534I/558W/681G/732W/739G/749F/750D/775G/1116I/1148N/1248D/1377D/1439E, 63Q/171P/212A/260T/306G/318G/320R/332P/387I/390A/393G/534I/558W/681G/732W/749F/750D/775G/800V/1148N/1248D/1377D/1439E, 63Q/171P/212A/260T/306G/318G/320R/332P/387I/390A/393G/534I/558W/681G/732W/749F/750D/775G/1093G/1148N/1248D/1377D/1439E, 63Q/171P/212A/260T/306G/318G/320R/332P/387I/390A/393G/534I/558W/681G/732W/749F/750D/752E/775G/1148N/1248D/1377D/1439E, 63Q/171P/212A/260T/306G/318G/320R/332P/387I/390A/393G/534I/558W/681G/732W/749F/750D/752E/775G/1093G/1148N/1248D/1377D/1439E, 63Q/171P/212A/260T/306G/318G/320R/332P/387I/390A/393G/534I/558W/681G/732W/749F/750D/752E/775G/799S/1148N/1248D/1377D/1439E, 63Q/171P/212A/260T/306G/318G/320R/332P/387I/390A/393G/534I/558W/681G/732W/749F/750D/775G/799S/809G/1093G/1148N/1248D/1377D/1439E, 63Q/171P/212A/260T/306G/318G/320R/332P/387I/390A/393G/534I/558W/681G/732W/749F/750D/775G/799S/1093G/1148N/1248D/1377D/1439E, 63Q/171P/212A/260T/306G/318G/320R/332P/387I/390A/393G/534I/558W/681G/732W/738E/739G/749F/750D/775G/1148N/1248D/1377D/1439E, 63Q/171P/212A/260T/306G/318G/320R/332P/387I/390A/393G/534I/558W/681G/732W/738E/739G/749F/750D/775G/800V/809G/1148N/1248D/1377D/1439E, 63Q/171P/212A/260T/306G/318G/320R/332P/387I/390A/393G/534I/558W/681G/732W/738E/749F/750D/775G/800V/1148N/1248D/1377D/1439E, 63Q/171P/212A/260T/306G/318G/320R/332P/387I/390A/393G/534I/558W/681G/732W/738E/749F/750D/775G/800V/805Q/809G/1148N/1248D/1377D/1439E, 63Q/171P/212A/260T/306G/318G/320R/332P/387I/390A/393G/534I/558W/681G/732W/738E/749F/750D/775G/809G/1148N/1248D/1377D/1439E, 63Q/171P/212A/260T/306G/318G/320R/332P/387I/390A/393G/534I/558W/681G/732W/738E/749F/750D/775G/809G/1093G/1148N/1248D/1377D/1439E, 63Q/171P/212A/260T/306G/318G/320R/332P/387I/390A/393G/534I/558W/681G/732W/738E/749F/750D/775G/809G/1093G/1116I/1148N/1248D/1377D/1439E, 63Q/171P/212A/260T/306G/318G/320R/332P/387I/390A/393G/534I/558W/681G/732W/738E/749F/750D/775G/809G/1116111148N/1248D/1377D/1439E, 63Q/171P/212A/260T/306G/320R/332P/387I/390A/393G/534I/558W/681G/732W/738E/749F/750D/775G/1148N/1248D/1377D/1439E, 63Q/171P/212A/260T/306G/320R/332P/387I/390A/393G/534I/558W/681G/732W/738E/749F/750D/775G/1076S/1148N/1248D/1377D/1439E, 63Q/171P/212A/260T/306G/320R/332P/387I/390A/393G/534I/558W/681G/732W/738E/749F/750D/775G/1076S/1116I/1148N/1248D/1377D/1439E, 63Q/171P/212A/260T/306G/320R/332P/387I/390A/393G/534I/558W/681G/732W/738E/739G/749F/750D/775G/805Q/808S/809G/1076S/1148N/1248D/1377D/1439E, 63Q/171P/212A/260T/306G/320R/332P/387I/390A/393G/I499L/534I/558W/681G/700E/732W/749F/750D/775G/809G/1148N/1248D/1377D/1439E, 63Q/171P/212A/260T/306G/320R/332P/387I/390A/393G/534I/558W/681G/732W/738E/749F/750D/775G/1093A/1148N/1248D/1377D/1439E, 63Q/171P/212A/260T/306G/320R/332P/387I/390A/393G/534I/558W/681G/732W/738E/749F/750D/752E/775G/1093A/1148N/1248D/1377D/1439E, 63Q/171P/212A/260T/306G/320R/332P/387I/390A/393G/534I/558W/681G/732W/738E/749F/750D/775G/1116I/1148N/1248D/1377D/1439E, 63Q/171P/212A/260T/306G/320R/332P/387I/390A/393G/534I/543P/558W/681G/732W/738E/739G/749F/75 OD/775G/1148N/1248D/1377D/1439E, 63Q/171P/212A/260T/306G/320R/332P/387I/390A/393G/534I/543P/558W/681N/732W/738E/749F/750D/752E/775G/1116I/1148N/1248D/1377D/1439E, 63Q/171P/212A/260T/306G/320R/332P/387I/390A/393G/534I/543P/558W/681G/732W/738E/749F/750D/775G/1093A/1148N/1248D/1377D/1439E, 63Q/171P/212A/260T/306G/320R/332P/387I/390A/393G/534I/558W/681G/700E/732W/749F/750D/775G/1093A/1148N/1248D/1377D/1414N/1439E, 63Q/171P/212A/260T/306G/318G/320R/332P/387I/390A/393G/534I/558W/681N/732W/738E/749F/750D/775G/1148N/1248D/1377D/1439E, 63Q/171P/212A/260T/306G/318G/320R/332P/387I/390A/393G/534I/558W/681N/732W/749F/750D/775G/1148N/1248D/1377D/1439E, 63Q/171P/212A/260T/306G/318G/320R/332P/387I/390A/393G/534I/558W/681N/732W/739G/749F/750D/775G/1093G/1148N/1248D/1377D/1439E, 63Q/171P/212A/260T/306G/318G/320R/332P/387I/390A/393G/534I/558W/681N/732W/739G/749F/750D/752E/775G/799S/800V/809G/1116I/1148N/1248D/1339C/1377D/1439E, 63Q/171P/212A/260T/306G/318G/320R/332P/387I/390A/393G/534I/558W/681N/732W/739G/749F/750D/775G/1116I/1148N/1248D/1377D/1439E, 63Q/171P/212A/260T/306G/318G/320R/332P/387I/390A/393G/534I/558W/681N/732W/749F/750D/775G/800V/1148N/1248D/1377D/1439E, 63Q/171P/212A/260T/306G/318G/320R/332P/387I/390A/393G/534I/558W/681N/732W/738E/749F/750D/775G/800V/805Q/809G/1148N/1248D/1377D/1439E, 63Q/171P/212A/260T/306G/318G/320R/332P/387I/390A/393G/534I/558W/681N/732W/738E/749F/750D/775G/809G/1093G/1148N/1248D/1377D/1439E, 63Q/171P/212A/260T/306G/318G/320R/332P/387I/390A/393G/534I/558W/681N/732W/738E/749F/750D/752E/775G/1093A/1148N/1248D/1377D/1439E, 63Q/171P/212A/260T/306G/318G/320R/332P/387I/390A/393G/534I/558W/681N/732W/738E/749F/750D/752E/775G/1093G/1148N/1248D/1377D/1439E, 63Q/171P/212A/260T/306G/318G/320R/332P/387I/390A/393G/534I/558W/681N/700E/732W/738E/749F/75 OD/775G/1148N/1248D/1377D/1439E, 63Q/171P/212A/260T/306G/318G/320R/332P/387I/390A/393G/534I/558W/681N/700E/732W/739G/749F/750D/775G/1093G/1116I/1148N/1248D/1377D/1439E, 63Q/171P/212A/260T/306G/318G/320R/332P/387I/390A/393G/534I/558W/681N/700E/732W/738E/749F/75 OD/775G/809G/1148N/1248D/1377D/1439E, 63Q/171P/212A/260T/306G/318G/320R/332P/387I/390A/393G/534I/558W/681N/700E/732W/738E/749F/75 OD/775G/1093G/1148N/1248D/1377D/1439E, 63Q/171P/212A/260T/306G/318G/320R/332P/387I/390A/393G/534I/558W/681N/700E/732W/738E/749F/75 OD/752E/775G/808S/809G/1093G/1116I/1148N/1248D/1377D/1439E, 63Q/171P/212A/260T/306G/318G/320R/332P/387I/390A/393G/534I/558W/681N/700E/732W/738E/749F/75 OD/752E/775G/1093G/1148N/1248D/1377D/1439E, 63Q/171P/212A/260T/306G/318G/320R/332P/387I/390A/393G/534I/558W/681N/700E/732W/738E/749F/75 OD/775G/844H/1148N/1248D/1377D/1439E, 63Q/171P/212A/260T/306G/318G/320R/332P/387I/390A/393G/534I/558W/681N/700E/732W/738E/749F/75 OD/775G/805Q/809G/1148N/1248D/1377D/1439E, 63Q/171P/212A/260T/306G/318G/320R/332P/387I/390A/393G/534I/558W/681G/732W/738E/749F/750D/775G/1093A/1148N/1248D/1377D/1439E, 63Q/171P/212A/260T/306G/318G/320R/332P/387I/390A/393G/534I/558W/681G/732W/738E/749F/750D/775G/1093G/1148N/1248D/1377D/1439E, 63Q/171P/212A/260T/306G/318G/320R/332P/387I/390A/393G/534I/558W/681G/732W/738E/749F/750D/775G/1093G/1116I/1148N/1248D/1377D/1439E, 63Q/171P/212A/260T/306G/318G/320R/332P/387I/390A/393G/534I/558W/681G/732W/738E/749F/750D/752E/775G/1148N/1248D/1377D/1439E, 63Q/171P/212A/260T/306G/318G/320R/332P/387I/390A/393G/534I/558W/681G/732W/738E/749F/750D/752E/775G/1076S/1148N/1248D/1377D/1439E, 63Q/171P/212A/260T/306G/318G/320R/332P/387I/390A/393G/534I/558W/681G/732W/738E/749F/750D/752E/775G/800V/809G/1148N/1248D/1377D/1439E, 63Q/171P/212A/260T/306G/318G/320R/332P/387I/390A/393G/534I/558W/681G/732W/738E/749F/750D/752E/775G/800V/1093G/1148N/1248D/1377D/1439E, 63Q/171P/212A/260T/306G/318G/320R/332P/387I/390A/393G/534I/558W/681G/732W/738E/749F/750D/752E/775G/809G/1148N/1248D/1377D/1439E, 63Q/171P/212A/260T/306G/318G/320R/332P/387I/390A/393G/534I/558W/681G/732W/738E/749F/750D/752E/775G/866A/1148N/1248D/1377D/1439E, 63Q/171P/212A/260T/306G/318G/320R/332P/387I/390A/393G/534I/558W/681G/732W/738E/749F/750D/752E/775G/1093G/1148N/1248D/1377D/1439E, 63Q/171P/212A/260T/306G/318G/320R/332P/387I/390A/393G/534I/558W/681G/732W/738E/749F/750D/752E/775G/1116I/1148N/1248D/1377D/1439E, 63Q/171P/178Q/212A/260T/306G/320R/332P/387I/390A/393G/534I/558W/681G/732W/738E/739G/749F/750D/752E/775G/809G/1076S/1116I/1148N/1248D/1377D/1439E, 63Q/171P/178Q/212A/260T/306G/320R/332P/387I/390A/393G/534I/558W/681G/732W/738E/749F/750D/775G/800V/809G/1148N/1248D/1377D/1439E, 63Q/171P/178Q/212A/260T/306G/320R/332P/387I/390A/393G/534I/558W/681N/732W/738E/749F/750D/775G/800V/808S/809G/1076S/1148N/1248D/1377D/1439E, 63Q/171P/178Q/212A/260T/306G/318G/320R/332P/387I/390A/393G/534I/558W/681N/732W/738E/749F/750D/775G/1148N/1248D/1377D/1439E, 63Q/171P/178Q/212A/260T/306G/318G/320R/332P/387I/390A/393G/534I/558W/681N/732W/749F/750D/775G/1116I/1148N/1248D/1377D/1439E, 63Q/171P/178Q/212A/260T/306G/318G/320R/332P/387I/390A/393G/534I/558W/681G/732W/738E/749F/750D/752E/775G/809G/1148N/1248D/1377D/1439E, 63Q/171P/178Q/212A/260T/306G/318G/320R/332P/387I/390A/393G/534I/558W/681G/700E/732W/738E/749F/750D/775G/800V/805Q/808S/809G/1148N/1248D/1377D/1439E, 63Q/171P/178Q/212A/260T/306G/318G/320R/332P/387I/390A/393G/534I/558W/681G/732W/738E/749F/750D/775G/805Q/809G/866A/1148N/1248D/1377D/1439E, 63Q/171P/212A/260T/306G/318G/320R/332P/387I/390A/393G/534I/558W/681G/732W/738E/749F/750D/775G/1116I/1148N/1248D/1377D/1439E, 63Q/171P/212A/260T/306G/318G/320R/332P/387I/390A/393G/534I/543P/558W/681G/732W/738E/749F/75 OD/775G/1148N/1248D/1377D/1439E, 63Q/171P/212A/260T/306G/318G/320R/332P/387I/390A/393G/534I/543P/558W/681G/732W/739G/749F/75 OD/775G/809G/1148N/1248D/1377D/1439E, 63Q/171P/212A/260T/306G/318G/320R/332P/387I/390A/393G/534I/543P/558W/681G/732W/749F/750D/775G/809G/1093G/1148N/1248D/1377D/1439E, 63Q/171P/212A/260T/306G/318G/320R/332P/387I/390A/393G/534I/543P/558W/681G/732W/738E/739G/749F/750D/775G/1093A/1148N/1248D/1377D/1439E, 63Q/171P/212A/260T/306G/318G/320R/332P/387I/390A/393G/534I/543P/558W/681G/732W/738E/749F/75 OD/775G/800V/1093G/1116I/1148N/1248D/1377D/1439E, 63Q/171P/212A/260T/306G/318G/320R/332P/387I/390A/393G/534I/543P/558W/681G/732W/738E/749F/75 OD/775G/809G/1093G/1148N/1248D/1377D/1439E, 63Q/171P/212A/260T/306G/318G/320R/332P/387I/390A/393G/534I/543P/558W/681G/732W/738E/749F/75 OD/775G/809G/1093G/1116I/1148N/1248D/1377D/1439E, 63Q/171P/212A/260T/306G/318G/320R/332P/387I/390A/393G/534I/543P/558W/681N/732W/739G/749F/75 OD/752E/775G/809G/1093G/1148N/1248D/1377D/1439E, 63Q/171P/212A/260T/306G/318G/320R/332P/387I/390A/393G/534I/543P/558W/681N/732W/739G/749F/75 OD/752E/775G/1093G/1148N/1248D/1377D/1439E, 63Q/171P/212A/260T/306G/318G/320R/332P/387I/390A/393G/534I/543P/558W/681N/732W/738E/739G/749F/750D/752E/775G/800V/805Q/809G/1093G/1148N/1248D/1377D/1439E, 63Q/171P/212A/260T/306G/318G/320R/332P/387I/390A/393G/534I/543P/558W/681N/700E/732W/739G/749F/750D/775G/800V/1093G/1116I/1148N/1248D/1377D/1439E, 63Q/171P/212A/260T/306G/318G/320R/332P/387I/390A/393G/534I/543P/558W/681N/700E/732W/739G/749F/750D/775G/805Q/808S/809G/1148N/1248D/1377D/1439E, 63Q/171P/212A/260T/306G/318G/320R/332P/387I/390A/393G/534I/543P/558W/681N/700E/732W/738E/749F/750D/752E/775G/1148N/1248D/1377D/1439E, 63Q/171P/212A/260T/306G/318G/320R/332P/387I/390A/393G/534I/543P/558W/681N/700E/732W/738E/749F/750D/752E/775G/809G/1093G/1148N/1248D/1377D/1439E, 63Q/171P/212A/260T/306G/318G/320R/332P/387I/390A/393G/534I/543P/558W/681G/732W/738E/749F/75 OD/775G/1093G/1148N/1248D/1377D/1439E, 63Q/171P/212A/260T/306G/318G/320R/332P/387I/390A/393G/534I/543P/558W/681G/700E/732W/738E/749F/750D/775G/1148N/1248D/1377D/1439E, 63Q/171P/212A/260T/306G/318G/320R/332P/387I/390A/393G/534I/543P/558W/681G/700E/732W/739G/749F/750D/752E/775G/800V/1093G/1116I/1148N/1248D/1377D/1439E, 63Q/171P/212A/260T/306G/318G/320R/332P/387I/390A/393G/534I/543P/558W/681G/700E/732W/738E/749F/750D/775G/809G/1148N/1248D/1377D/1439E, 63Q/171P/212A/260T/306G/318G/320R/332P/387I/390A/393G/534I/543P/558W/681G/700E/732W/738E/749F/750D/775G/1093G/1148N/1248D/1377D/1439E, 63Q/171P/212A/260T/306G/318G/320R/332P/387I/390A/393G/534I/543P/558W/681G/700E/732W/738E/749F/750D/752E/775G/1148N/1248D/1377D/1439E, 63Q/171P/212A/260T/306G/318G/320R/332P/387I/390A/393G/534I/543P/558W/681G/700E/732W/738E/749F/750D/752E/775G/800V/1093G/1148N/1248D/1377D/1439E, 63Q/171P/212A/260T/306G/318G/320R/332P/387I/390A/393G/534I/543P/558W/681G/700E/732W/738E/749F/750D/752E/775G/809G/1116I/1148N/1248D/1377D/1439E, 63Q/171P/212A/260T/306G/318G/320R/332P/387I/390A/393G/534I/543P/558W/681G/700E/732W/738E/749F/750D/752E/775G/1093G/1148N/1248D/1377D/1439E, 63Q/171P/212A/260T/306G/318G/320R/332P/387I/390A/393G/534I/558W/681G/700E/732W/738E/749F/75 OD/775G/1148N/1248D/1377D/1439E, 63Q/171P/212A/260T/306G/318G/320R/332P/387I/390A/393G/534I/558W/681G/700E/732W/739G/749F/750D/775G/800V/1093G/1148N/1248D/1377D/1439E, 63Q/171P/212A/260T/306G/318G/320R/332P/387I/390A/393G/534I/558W/681G/700E/732W/739G/749F/750D/775G/799S/1093G/1148N/1248D/1377D/1439E, 63Q/171P/212A/260T/306G/318G/320R/332P/387I/390A/393G/534I/558W/681G/700E/732W/749F/750D/775G/800V/1093G/1148N/1248D/1377D/1439E, 63Q/171P/212A/260T/306G/318G/320R/332P/387I/390A/393G/534I/558W/681G/700E/732W/749F/750D/775G/1116I/1148N/1248D/1377D/1439E, 63Q/171P/212A/260T/306G/318G/320R/332P/387I/390A/393G/534I/558W/681G/700E/732W/738E/749F/75 OD/775G/809G/1148N/1248D/1377D/1439E, 63Q/171P/212A/260T/306G/318G/320R/332P/387I/390A/393G/534I/558W/681G/700E/732W/738E/749F/75 OD/775G/809G/1093G/1148N/1248D/1377D/1439E, 63Q/171P/212A/260T/306G/318G/320R/332P/387I/390A/393G/534I/558W/681G/700E/732W/738E/749F/75 OD/775G/1093G/1148N/1248D/1377D/1439E, 63Q/171P/212A/260T/306G/318G/320R/332P/387I/390A/393G/534I/558W/681G/700E/732W/738E/749F/75 OD/775G/1093G/1116I/1148N/1248D/1377D/1439E, 63Q/171P/212A/260T/306G/318G/320R/332P/387I/390A/393G/534I/558W/681G/700E/732W/738E/749F/75 OD/752E/775G/1148N/1248D/1377D/1439E, 63Q/171P/212A/260T/306G/318G/320R/332P/387I/390A/393G/534I/558W/681G/700E/732W/738E/749F/75 OD/752E/775G/808S/809G/1093G/1116I/1148N/1248D/1377D/1439E, 63Q/171P/212A/260T/306G/318G/320R/332P/387I/390A/393G/534I/558W/681G/700E/732W/738E/749F/75 OD/752E/775G/809G/1093G/1148N/1248D/1377D/1439E, 63Q/171P/212A/260T/306G/318G/320R/332P/387I/390A/393G/534I/558W/681G/700E/732W/738E/749F/75 OD/752E/775G/799S/1116I/1148N/1248D/1377D/1439E, 63Q/171P/212A/260T/306G/318G/320R/332P/387I/390A/393G/534I/558W/681G/700E/732W/738E/749F/75 OD/752E/775G/805Q/809G/1093A/1148N/1248D/1377D/1439E, 63Q/171P/212A/260T/306G/318G/320R/332P/387I/390A/393G/534I/558W/681G/700E/732W/738E/749F/75 OD/775G/799S/800V/1148N/1248D/1377D/1439E, 63Q/171P/212A/260T/306G/318G/320R/332P/387I/390A/393G/534I/558W/681G/700E/732W/738E/749F/75 OD/775G/1116I/1148N/1248D/1377D/1439E, 63Q/171P/212A/260T/306G/318G/320R/332P/387I/390A/393G/534I/558W/681G/700E/732W/738E/749F/75 OD/775G/805Q/808S/809G/1076S/1148N/1248D/1377D/1439E, 63Q/171P/212A/260T/306G/318G/320R/332P/387I/390A/393G/514L/534I/543P/558W/681N/732W/738E/749F/750D/775G/805Q/808S/809G/1148N/1248D/1377D/1439E, 63Q/171P/212A/260T/306G/318G/320R/332P/387I/390A/393G/534I/558W/681G/732W/738E/749F/750D/775G/805Q/808S/809G/1148N/1248D/1377D/1439E, or 63Q/171P/212A/260T/306G/318G/320R/332P/387I/390A/393G/534I/558W/681G/732W/738E/749F/750D/775G/805Q/809G/1116I/1148N/1248D/1377D/1439E, wherein the amino acid positions are relative to the reference sequence corresponding to SEQ ID NO: 2.

In some embodiments, the amino acid sequence of the recombinant factor VIII polypeptide comprises a substitution at an amino acid position set forth in Tables 4.1, 4.2, 5.1, 5.2, 6.1, 6.2, 7.1, 7.2, 8.1, 8.2, 9.1, 9.2, 10.1, 10.2, 11.1, and 15.1, wherein the amino acid positions are relative to the reference sequence corresponding to SEQ ID NO: 2.

In some embodiments, the amino acid sequence of the recombinant factor VIII polypeptide comprises at least one substitution set forth in Tables 4.1, 4.2, 5.1, 5.2, 6.1, 6.2, 7.1, 7.2, 8.1, 8.2, 9.1, 9.2, 10.1, 10.2, 11.1, and 15.1, wherein the amino acid positions are relative to the reference sequence corresponding to SEQ ID NO: 2.

In some embodiments, the amino acid sequence of the recombinant factor VIII polypeptide comprises at least a substitution or substitution set at the position(s) set forth in Tables 4.1, 4.2, 5.1, 5.2, 6.1, 6.2, 7.1, 7.2, 8.1, 8.2, 9.1, 9.2, 10.1, 10.2, 11.1, and 15.1, wherein the amino acid positions are relative to the reference sequence corresponding to SEQ ID NO: 2.

In some embodiments, the amino acid sequence of the recombinant factor VIII polypeptide comprises at least a substitution or substitution set of a variant set forth in Tables 4.1, 4.2, 5.1, 5.2, 6.1, 6.2, 7.1, 7.2, 8.1, 8.2, 9.1, 9.2, 10.1, 10.2, 11.1, and 15.1, wherein the amino acid positions are relative to the reference sequence corresponding to SEQ ID NO: 2.

In some embodiments, the recombinant factor VIII polypeptide comprises an amino acid sequence having at least 70%, 75%, 80%, 81%, 82%, 83%, 84%, 85%, 86%, 87%, 88%, 89%, 90%, 91%, 92%, 93%, 94%, 95%, 96%, 97%, 98%, or 99% or more sequence identity to the reference sequence corresponding to residues 20-1457 of SEQ ID NO: 252, 504, 706, 840, 1200, 1514, or 2072, or to the reference sequence corresponding to SEQ ID NO: 252, 504, 706, 840, 1200, 1514, or 2072.

In some embodiments, the recombinant factor VIII polypeptide comprises an amino acid sequence having at least 70%, 75%, 80%, 81%, 82%, 83%, 84%, 85%, 86%, 87%, 88%, 89%, 90%, 91%, 92%, 93%, 94%, 95%, 96%, 97%, 98%, or 99% or more sequence identity to the reference sequence corresponding to residues 20-1457 of SEQ ID NO: 252, 504, 706, 840, 1200, 1514, or 2072, or to the reference sequence corresponding to SEQ ID NO: 252, 504, 706, 840, 1200, 1514, or 2072, with the proviso that the recombinant factor VIII polypeptide does not include the amino acid sequence of SEQ ID NO: 2.

In some embodiments, the recombinant factor VIII polypeptide comprises an amino acid sequence having at least 70%, 75%, 80%, 81%, 82%, 83%, 84%, 85%, 86%, 87%, 88%, 89%, 90%, 91%, 92%, 93%, 94%, 95%, 96%, 97%, 98%, or 99% or more sequence identity to the reference sequence corresponding to residues 20-1457 of an even-numbered SEQ ID NO. of SEQ ID NOs. 4-2346 and 2360-2870, or to the reference sequence corresponding to an even-numbered SEQ ID NO. of SEQ ID NOs. 4-2346 and 2350-2870.

In some embodiments, the recombinant factor VIII polypeptide comprises an amino acid sequence having at least 70%, 75%, 80%, 81%, 82%, 83%, 84%, 85%, 86%, 87%, 88%, 89%, 90%, 91%, 92%, 93%, 94%, 95%, 96%, 97%, 98%, or 99% or more sequence identity to the reference sequence corresponding to residues 20-1457 of SEQ ID NO: 252, 504, 706, 840, 1200, 1514, or 2072, or to the reference sequence corresponding to SEQ ID NO: 252, 504, 706, 840, 1200, 1514, or 2072, wherein the amino acid sequence comprises one or more substitutions relative to the reference sequence corresponding to residues 20-1457 of SEQ ID NO: 252, 504, 706, 840, 1200, 1514, or 2072, or relative to the reference sequence corresponding to SEQ ID NO: 252, 504, 706, 840, 1200, 1514, or 2072.

In some embodiments, the amino acid sequence of the recombinant factor VIII polypeptide comprises at least a substitution at amino acid position 13, 14, 15, 16, 17, 18, 19, 20, 21, 24, 37, 56, 57, 58, 59, 61, 63, 64, 65, 85, 96, 104, 110, 119, 127, 132, 158, 160, 164, 167, 168, 171, 175, 178, 186, 187, 205, 210, 211, 212, 215, 225, 246, 257, 260, 265, 281, 292, 294, 301, 303, 304, 306, 308, 310, 311, 312, 318, 320, 321, 325, 328, 332, 334, 335, 344, 346, 347, 349, 351, 353, 354, 355, 356, 358, 359, 362, 370, 387, 388, 390, 392, 393, 397, 407, 428, 429, 433, 435, 436, 481, 487, 488, 489, 491, 498, 499, 503, 514, 527, 533, 534, 535, 536, 538, 543, 550, 551, 555, 558, 569, 580, 581, 584, 610, 611, 612, 613, 614, 615, 616, 617, 618, 619, 620, 621, 622, 643, 646, 650, 665, 667, 676, 680, 681, 682, 683, 684, 686, 693, 696, 698, 700, 706, 707, 708, 712, 716, 724, 726, 729, 731, 732, 734, 735, 736, 737, 738, 739, 740, 744, 746, 747, 748, 749, 750, 751, 752, 753, 754, 755, 756, 757, 759, 770, 773, 775, 785, 786, 787, 790, 791, 792, 793, 794, 795, 797, 798, 799, 800, 801, 802, 805, 806, 807, 808, 809, 810, 811, 812, 815, 816, 817, 818, 819, 821, 822, 824, 825, 826, 828, 830, 831, 836, 837, 838, 839, 840, 841, 844, 866, 873, 879, 892, 896, 897, 903, 907, 914, 916, 917, 918, 919, 921, 922, 923, 924, 926, 929, 938, 939, 945, 948, 951, 968, 999, 1037, 1040, 1041, 1042, 1043, 1049, 1050, 1051, 1053, 1054, 1076, 1078, 1080, 1084, 1088, 1089, 1092, 1093, 1094, 1095, 1098, 1099, 1100, 1101, 1104, 1108, 1109, 1113, 1114, 1116, 1127, 1129, 1138, 1145, 1148, 1151, 1156, 1158, 1160, 1186, 1192, 1193, 1194, 1205, 1210, 1218, 1220, 1221, 1222, 1224, 1229, 1245, 1248, 1250, 1259, 1264, 1269, 1270, 1274, 1281, 1283, 1292, 1297, 1339, 1352, 1373, 1377, 1378, 1380, 1389, 1414, 1422, 1427, 1428, 1439, 1440, 1442, or 1452, or combinations thereof, wherein the amino acid positions are relative to the reference sequence corresponding to SEQ ID NO: 252, 504, 706, 840, 1200, 1514, or 2072.

In some embodiments, the amino acid sequence of the recombinant factor VIII polypeptide comprises at least a substitution or an amino acid residue 13G/Y, 14G/M/V, 15G/S, 16A/D/G/P/V, 17S/V, 18L/R, 19G, 20L, 21M, 24V, 37H, 56E/P, 57A/L/S/T/V, 58K, 59D/G/L/M/Q/R/S/V/W, 61Q/S, 63C/E/F/G/H/I/K/L/M/N/Q/R/S/T/W/Y, 64D/G, 65A, 85Q, 96I/L/R/V/W, 104S, 110H, 119G/V, 127V, 132P/R, 158G, 160E/T, 164E, 167G/H/R, 168G/R, 171A/D/E/F/H/K/N/M/P/Q/R/S/T/V/W/Y, 175A/G/H, 178C/M/Q/V/W, 186A, 187C/G, 205R, 210P, 211G/K/P/Q/R, 212A/F/G/L/K/M/N/P/R/S/V/Y, 215E/P/S, 225R, 246D, 257I, 260E/K/T, 265R/S/W, 281A/S, 292D, 294G, 301K/M, 303G/T, 304A/E/G, 306A/D/G/P/S/T, 308P, 310G/V, 311E/G, 312L, 318C/G/K/L/M/Q/R/T/V, 320G/I/L/P/Q/R/S/T/V, 321Y, 325Y, 328G/Q/S, 332A/F/G/H/N/P/Q/R/T/V/W/Y, 334G, 335E/P, 344A/E/L, 346R/V, 347N, 349G, 351S, 353S, 354R, 355C, 356L/N, 358K/S, 359E/G, 362E/G/N/P/T/V/W, 370A, 387A/G/I/L/Q/R/S/T/V/W, 388A/P, 390A/E/G/L/M/P/S/T/V, 392A, 393A/F/G/H/M/Q/R/W/Y, 397G/N, 407V, 428F, 429L/S, 433A/G, 435Q, 436D/G, 481V, 487N, 488G, 489E/F/Q/R, 491E, 498A, 499C/L, 503S, 514L, 527A, 533R/V/Y, 534I/F/L/M, 535C/G/N, 536A/G/K/L/M/P/R/W, 538V, 543G/L/P/W, 550G, 551W, 555E/G/R/T, 558A/E/G/L/P/Q/R/S/T/V/W, 569T, 580S/Y, 581K/V, 584A, 610H/K/L/R/S/T, 611D/E, 612G/S/T, 613S, 614A/F/H/I/N/T, 615D/N, 616D/E/G/Q/W, 617D/E/G/N, 618C/E/G/P/Y, 619E, 620N/P, 621K/M/R/T/W, 622D, 643N, 646I, 650M, 665G, 667I, 676Q, 680L/R, 681A/C/E/F/G/L/N/R/Q/S/T/W, 682R, 683W, 684G/K, 686W, 693T, 696E, 698D/S/W/Y, 700A/E/Q/T/V, 706A/G/M/Y, 707H, 708A/K/T/V, 712G/L/M/Q/S/T/Y, 716K/L/W, 724I, 726A/L, 729T, 731I/L/R, 732C/D/E/G/L/R/W/K/M/Q/S/T/V/Y, 734I/R/S, 735D/S/V, 736F/G, 737F/G/K/L/M/N/S/V, 738A/D/E/F/H/K/N/P/R/S/T/Y, 739G/P/R, 740G, 744A/R, 746P, 747D, 748F/G/K/N/S/V, 749A/C/D/F/G/I/K/L/M/P/Q/R/S/T/V/Y, 750A/D/G/K/M/N/P/Q/R/S/T/V/W, 751A/D/E/Y, 752D/E/F/G/L/M/Q/R/S/V, 753D, 754W, 755G/L/P/Q/S/Y, 756G/P/R/T, 757A/G/K/T, 759S, 770W, 773H, 775E/G/K/N/P/S/V, 785V, 786C/D/L, 787W, 790A/G/I/M/P/W, 791M/T, 792H/W/Y, 793G, 794R, 795C/N/S/T, 797C/G/P/Q/T/W/Y, 798C/R/T, 799A/E/G/M/P/Q/R/S/V, 800C/G/I/L/M/N/P/Q/R/T/V/W/Y, 801A/G/I/R, 802S/V, 805C/G/H/K/L/M/P/Q/R/S/V/W, 806E/G/R/S, 807C/L/P/R, 808K/S, 809A/C/G/I/K/L/N/Q/R/S/T/V/W, 810V, 811F, 812G/I/V, 815I, 816V/W, 817A/D, 818A/E/G/P/S/V/W, 819V, 821Q, 822S/T, 824W, 825L, 826G, 828K, 830M/V, 831W, 836G/R/V/W, 837G, 838G/T, 839A/G/K/P, 840G, 841W, 844H, 866A, 873L/Q/T, 879Q, 892T, 896L/V, 897G/K/R, 903E, 907D, 914V, 916C/E/I/K/L/M/Q/T, 917G, 918M/S, 919G, 921R/W, 922W, 923M, 924I/M/P/Q/W, 926T/V, 929A/Q/V, 938R/V, 939E/S, 945K, 948S, 951V, 968A/P, 999R, 1037H/M/N/P, 1040E, 1041G/H/N, 1042A/E/T, 1043E/R, 1049G/W, 1050E/P/Q/S/T, 1051E/H/K/R/S/W, 1053A/R, 1054C, 1076G/S, 1078S, 1080G/V, 1084L/N, 1088G/S, 1089F/S, 1092G, 1093A/E/G/M/N/P/Q/R/S/T/V/W/Y, 1094S, 1095G, 1098G, 1099G, 1100I/S, 1101G, 1104C/E, 1108M/S/V/W, 1109G, 1113F, 1114A/M, 1116A/G/I/L/R, 1127L/V, 1129P, 1138Q, 1145R, 1148A/C/E/L/M/N/Q/R/S, 1151S, 1156D/G/N/R, 1158N, 1160R, 1186E, 1192D, 1193A/E/G/I/L, 1194P, 1205L, 1210M/T/V/W, 1218A/G/L/M/P, 1220D/E/L/T, 1221G/S/T/V, 1222F, 1224P, 1229D, 1245R, 1248A/C/D/E/F/G/H/I/K/M/P/Q/R/S/T/W/Y, 1250A/P/S/T, 1259T, 1264L/R/W, 1269F, 1270V, 1274N/V, 1281H/K/P/Q/R/S, 1283E/H/L/S/T, 1292Y, 1297A, 1339C, 1352E, 1373S, 1377A/C/D/E/G/H/I/M/P/R/S/V/T/Y, 1378A/C/D/E, 1380A/G/W, 1389W, 1414A/C/D/E/G/H/N/P/T/W, 1422I, 1427Y, 1428G, 1439A/E/H/L/S/T, 1440A/C/E/G/S/T, or 1442T, or combinations thereof, wherein the amino acid positions are relative to the reference sequence corresponding to SEQ ID NO: 252, 504, 706, 840, 1200, 1514, or 2072.

In some embodiments, the amino acid sequence of the recombinant factor VIII polypeptide comprises at least a substitution at amino acid position 63, 171, 212, 260, 306, 317, 320, 332, 387, 390, 393, 534, 558, 681, 732, 738, 749, 750, 775, 1148, 1248, 1377, or 1439, or combinations thereof, wherein the amino acid positions are relative to the reference sequence corresponding to SEQ ID NO: 252, 504, 706, 840, 1200, 1514, or 2072.

In some embodiments, the amino acid sequence of the recombinant factor VIII polypeptide comprises at least a substitution or an amino acid residue 63C/E/F/G/H/I/K/L/M/N/Q/R/S/T/W/Y, 171A/D/E/F/H/K/N/M/P/Q/R/S/T/V/W/Y, 212A/F/G/L/K/M/N/P/R/S/V/Y, 260E/K/T, 306A/D/G/P/S/T, 318C/G/K/L/M/Q/R/T/V, 320G/I/L/P/Q/R/S/T/V, 332A/F/G/H/N/P/Q/R/T/V/W/Y, 387A/G/I/L/Q/R/S/T/V/W, 390A/E/G/L/M/P/S/T/V, 393A/F/G/H/M/Q/R/W/Y, 499C/L, 534I/F/L/M, 558A/E/G/L/P/Q/R/S/T/V/W, 681A/C/E/F/G/L/N/R/Q/S/T/W, 700A/E/Q/T/V, 732C/D/E/G/L/R/W/K/M/Q/S/T/V/Y, 738A/D/E/F/H/K/N/P/R/S/T/Y, 749A/C/D/F/G/I/K/L/M/P/Q/R/S/T/V/Y, 750A/D/G/K/M/N/P/Q/R/S/T/V/W, 752D/E/F/G/L/M/Q/R/S/V, 775E/G/K/N/P/S/V, 800C/G/I/L/M/N/P/Q/R/T/V/W/Y, 805C/G/H/K/L/M/P/Q/R/S/V/W, 809A/C/G/I/K/L/N/Q/R/S/T/V/W, 1093A/E/G/M/N/P/Q/R/S/T/V/W/Y, 1148A/C/E/L/M/N/Q/R/S, 1248A/C/D/E/F/G/H/I/K/M/P/Q/R/S/T/W/Y, 1377A/C/D/E/G/H/I/M/P/R/S/V/T/Y, 1414A/C/D/E/G/H/N/P/T/W, or 1439A/E/H/L/S/T, or combinations thereof, wherein the amino acid positions are relative to the reference sequence corresponding to SEQ ID NO: 252, 504, 706, 840, 1200, 1514, or 2072.

In some embodiments, the amino acid sequence of the recombinant factor VIII polypeptide comprises at least a substitution or an amino acid residue 63Q, 171P, 212A, 260T, 306G, 317G, 320R, 332P, 387I, 390A, 393G, 534I, 558W, 681G, 732W, 7338E, 749F, 750D, 775G, 1148N, 1248D, 1377D, or 1439E, or combinations thereof, wherein the amino acid positions are relative to the reference sequence corresponding to SEQ ID NO: 252, 504, 706, 840, 1200, 1514, or 2072.

In some embodiments, the recombinant factor VIII polypeptide comprises an amino acid sequence having at least 70%, 75%, 80%, 81%, 82%, 83%, 84%, 85%, 86%, 87%, 88%, 89%, 90%, 91%, 92%, 93%, 94%, 95%, 96%, 97%, 98%, or 99% or more sequence identity to the reference sequence corresponding to residues 20-1457 of SEQ ID NO: 252, or to the reference sequence corresponding to SEQ ID NO: 252, wherein the amino acid sequence comprises one or more substitutions relative to the reference sequence corresponding to residues 20-1457 of SEQ ID NO: 252, or relative to the reference sequence corresponding to SEQ ID NO: 252.

In some embodiments, the amino acid sequence of the recombinant factor VIII polypeptide comprises at least a substitution or substitution set at amino acid position(s) 1380, 388, 1043, 610, 1274, 1220, 1442, 1193, 621, 1054, 755, 1041, 748, 1049, 1221, 1051, 612, 1037, 616, 387, 489, 750, 1224, 618, 1050, 1053, 896, 897, 749, 260, 481, 613, 756, 63, 265, 752, 1218, 1378, 614, 1440, 1439, 1205, 1222, 1389, 1269, 390, 397/1218, 757, 1373, 346, 63/1248, 349, 306/355/1422, 1422, 306/346/708, 1148, 171/346/1248, 63/797/799/1248/1422, 171/799, 362, 1248, 171/306/346/999/1248, 171/1148, 734/799/1248/1422, 63/732, 63/171/306/346/732, 171, 171/1422, 63/362/732/799/1248, 999/1148/1422, 346/349, 306/349, 63/171/1148/1151, 306, 344/349/354/1250, 1250, 63/306/344/349/354/503/1248, 59/63/332/349/503/706/708, 63/306/344/349/354/706/1248, 59/63, 332/706/708/1248/1250, 503, 59/63/349/354, 59/349/708, 59/306/344/1248, 344/1248/1250, 708, 63/344/349, 59, 63/344/349/354, 706, 59/306/770, 63/347/354/1248, or 354/503, wherein the amino acid positions are relative to the reference sequence corresponding to SEQ ID NO: 252.

In some embodiments, the amino acid sequence of the recombinant factor VIII polypeptide comprises at least a substitution or substitution set 1380W, 388A, 1043E, 1043R, 610R, 1274N, 1220T, 1442T, 1193L, 621T, 1054C, 755Q, 1041G, 748S, 1049G, 1221V, 1051E, 612G, 1037H, 616E, 1041H, 387S, 489R, 621M, 489Q, 750G, 1224P, 618C, 616G, 612T, 1050Q, 1053A, 616W, 896V, 489F, 1050P, 897G, 749T, 1274V, 1220L, 260E, 481V, 613S, 387G, 621R, 756R, 63R, 265S, 1221T, 1380G, 752G, 748K, 755L, 1218A, 748N, 1378E, 614I, 749F, 387V, 750T, 1193G, 749V, 1378A, 750V, 387T, 387Q, 750A, 1037N, 1440A, 1051S, 1221G, 1378D, 1440S, 387R, 1193A, 387I, 1218M, 265W, 1440E, 1439L, 1440T, 612S, 1049W, 1221S, 1220D, 749G, 749S, 1218G, 1205L, 1222F, 1193E, 1050S, 1389W, 1439E, 260T, 1037P, 1051K, 1269F, 265R, 1051R, 1218L, 897R, 388P, 748G, 756P, 748F, 390A, 610L, 755G, 749R, 748V, 1380A, 1440C, 397N/1218M, 1037M, 1218P, 757K, 1373S, 1440G, 1193I, 1439T, 750D, 390G, 756G, 1220E, 1378C, 618G, 346R, 63Q/1248D, 349G, 306G/355C/1422I, 1422I, 306G/346R/708T, 1148N, 171P/346R/1248D, 63Q/797T/799R/1248D/1422I, 171P/799R, 362P, 1248D, 63Q, 171P/306G/346R/999R/1248D, 171P/1148N, 734S/799R/1248D/1422I, 63Q/732E, 63Q/171P/306G/346R/732G, 171P, 171P/1422I, 63Q/362P/732G/799R/1248D, 999R/1148N/1422I, 346R/349G, 306G/349G, 63Q/171P/1148N/1151S, 306G, 344E/349G/354R/1250T, 63G, 1250T, 63R/306G/344E/349G/354R/503S/1248R, 59D/63S/332P/349G/503S/706M/708K, 63S/306G/344A/349G/354R/706M/1248R, 1248R, 59D/63G, 332P/706M/708K/1248R/1250T, 503S, 59D/63R/349G/354R, 59D/349G/708K, 59D/306G/344E/1248R, 344E/1248R/1250T, 708K, 63R/344E/349G, 59D, 63S/344A/349G/354R, 706M, 59D/306G/770W, 63G/347N/354R/1248R, or 354R/503S, wherein the amino acid positions are relative to the reference sequence corresponding to SEQ ID NO: 252.

In some embodiments, the amino acid sequence of the recombinant factor VIII polypeptide comprises at least a substitution or substitution set M1380W, I388A, F1043E, F1043R, I610R, I1274N, S1220T, I1442T, F1193L, Q621T, L1054C, A755Q, Y1041G, Y748S, Y1049G, L1221V, M1051E, R612G, F1037H, N616E, Y1041H, F387S, S489R, Q621M, S489Q, L750G, S1224P, A618C, N616G, R612T, I1050Q, T1053A, N616W, I896V, S489F, I1050P, M897G, L749T, I1274V, S1220L, S260E, L481V, F613S, F387G, Q621R, I756R, V63R, I265S, L1221T, M1380G, K752G, Y748K, A755L, F1218A, Y748N, T1378E, L614I, L749F, F387V, L750T, F1193G, L749V, T1378A, L750V, F387T, F387Q, L750A, F1037N, H1440A, M1051S, L1221G, T1378D, H1440S, F387R, F1193A, F387I, F1218M, I265W, H1440E, V1439L, H1440T, R612S, Y1049W, L1221S, S1220D, L749G, L749S, F1218G, I1205L, Y1222F, F1193E, I1050S, S1389W, V1439E, S260T, F1037P, M1051K, I1269F, I265R, M1051R, F1218L, M897R, I388P, Y748G, I756P, Y748F, I390A, I610L, A755G, L749R, Y748V, M1380A, H1440C, H397N/F1218M, F1037M, F1218P, E757K, V1373S, H1440G, F1193I, V1439T, L750D, I390G, I756G, S1220E, T1378C, A618G, D346R, V63Q/L1248D, P349G, E306G/R355C/L1422I, L1422I, E306G/D346R/I708T, T1148N, L171P/D346R/L1248D, V63Q/M797T/K799R/L1248D/L1422I, L171P/K799R, A362P, L1248D, V63Q, L171P/E306G/D346R/Q999R/L1248D, L171P/T1148N, T734S/K799R/L1248D/L1422I, V63Q/K732E, V63Q/L171P/E306G/D346R/K732G, L171P, L171P/L1422I, V63Q/A362P/K732G/K799R/L1248D, Q999R/T1148N/L1422I, D346R/P349G, E306G/P349G, V63Q/L171P/T1148N/G1151S, E306G, K344E/P349G/L354R/V1250T, V63G, V1250T, V63R/E306G/K344E/P349G/L354R/R503S/L1248R, F59D/V63S/S332P/P349G/R503S/L706M/I708K, V63S/E306G/K344A/P349G/L354R/L706M/L1248R, L1248R, F59D/V63G, S332P/L706M/I708K/L1248R/V1250T, R503S, F59D/V63R/P349G/L354R, F59D/P349G/I708K, F59D/E306G/K344E/L1248R, K344E/L1248R/V1250T, I708K, V63R/K344E/P349G, F59D, V63S/K344A/P349G/L354R, L706M, F59D/E306G/R770W, V63G/S347N/L354R/L1248R, or L354R/R503S, wherein the amino acid positions are relative to the reference sequence corresponding to SEQ ID NO: 252.

In some embodiments, the recombinant factor VIII polypeptide comprises an amino acid sequence having at least 70%, 75%, 80%, 81%, 82%, 83%, 84%, 85%, 86%, 87%, 88%, 89%, 90%, 91%, 92%, 93%, 94%, 95%, 96%, 97%, 98%, or 99% or more sequence identity to the reference sequence corresponding to residues 20-1457 of SEQ ID NO: 504, or to the reference sequence corresponding to SEQ ID NO: 504, wherein the amino acid sequence comprises one or more substitutions relative to the reference sequence corresponding to residues 20-1457 of SEQ ID NO: 504, or relative to the reference sequence corresponding to SEQ ID NO: 504.

In some embodiments, the amino acid sequence of the recombinant factor VIII polypeptide comprises at least a substitution or substitution set at amino acid position(s) 750, 1051, 260, 756, 1193, 332, 897, 387, 1037, 306/749, 1270, 489, 487, 488, 903, 171/1248/1440, 260/1148/1378, 171/260/387/750/1248, 171/260, 171/260/387/750/1051/1148, 171, 750/1051/1148, 171/260/756, 171/1248, 171/1148, 59, 1440, 332/750/1248, 756/1248, 1248, 171/387/1248/1440, 750/1440, 59/171/750, 171/1193/1248, 750/1148/1193/1440, 1148, 171/387/1051, 748, 306, 306/387/390/749/1220, 390, 306/390/1037, 306/612/749, 387/390/749, 63/390/503/749, 1220, 63/306, 749, 306/390/749/1037, 306/749/1220, 306/387/390/612/749/1037, or 328, wherein the amino acid positions are relative to the reference sequence corresponding to SEQ ID NO: 504.

In some embodiments, the amino acid sequence of the recombinant factor VIII polypeptide comprises at least a substitution or substitution set 750D, 1051K, 1051S, 260T, 756P, 1193A, 332P, 897R, 387Q, 1037N, 387R, 306G/749F, 1270V, 489E, 487N, 488G, 903E, 171P/1248D/1440T, 260T/1148N/1378D, 171P/260T/387I/750D/1248D, 171P/260T, 171P/260T/387I/750D/1051S/1148N, 171P, 750D/1051S/1148N, 171P/260T/756P, 171P/1248D, 171P/1148N, 387I, 59D, 1440S, 332P/750D/1248D, 756P/1248D, 1248D, 171P/387I/1248D/1440S, 750D/1440S, 59D/171P/750D, 171P/1193A/1248D, 750D/1148N/1193A/1440S, 1148N, 171P/387I/1051K, 748G, 306G, 306G/387R/390A/749F/1220D, 390A, 306G/390A/1037N, 306G/612S/749F, 387V/390A/749F, 63S/390A/503S/749F, 387R/390A/749R, 1220D, 63S/306G, 749F, 306G/390A/749F/1037N, 387V, 306G/749F/1220D, 306G/387V/390A/612S/749F/1037N, or 328S, wherein the amino acid positions are relative to the reference sequence corresponding to SEQ ID NO: 504.

In some embodiments, the amino acid sequence of the recombinant factor VIII polypeptide comprises at least a substitution or substitution set L750D, M1051K, M1051S, S260T, I756P, F1193A, S332P, M897R, F387Q, F1037N, F387R, E306G/L749F, I1270V, S489E, Q487N, A488G, Q903E, L171P/L1248D/H1440T, S260T/T1148N/T1378D, L171P/S260T/F387I/L750D/L1248D, L171P/S260T, L171P/S260T/F387I/L750D/M1051S/T1148N, L171P, L750D/M1051S/T1148N, L171P/S260T/I756P, L171P/L1248D, L171P/T1148N, F387I, F59D, H1440S, S332P/L750D/L1248D, I756P/L1248D, L1248D, L171P/F387I/L1248D/H1440S, L750D/H1440S, F59D/L171P/L750D, L171P/F1193A/L1248D, L750D/T1148N/F1193A/H1440S, T1148N, L171P/F387I/M1051K, Y748G, E306G, E306G/F387R/I390A/L749F/S1220D, I390A, E306G/I390A/F1037N, E306G/R612S/L749F, F387V/I390A/L749F, V63S/I390A/R503S/L749F, F387R/I390A/L749R, S1220D, V63S/E306G, L749F, E306G/I390A/L749F/F1037N, F387V, E306G/L749F/S1220D, E306G/F387V/I390A/R612S/L749F/F1037N, or F328S, wherein the amino acid positions are relative to the reference sequence corresponding to SEQ ID NO: 504.

In some embodiments, the recombinant factor VIII polypeptide comprises an amino acid sequence having at least 70%, 75%, 80%, 81%, 82%, 83%, 84%, 85%, 86%, 87%, 88%, 89%, 90%, 91%, 92%, 93%, 94%, 95%, 96%, 97%, 98%, or 99% or more sequence identity to the reference sequence corresponding to residues 20-1457 of SEQ ID NO: 706, or to the reference sequence corresponding to SEQ ID NO: 706, wherein the amino acid sequence comprises one or more substitutions relative to the reference sequence corresponding to residues 20-1457 of SEQ ID NO: 706, or relative to the reference sequence corresponding to SEQ ID NO: 706.

In some embodiments, the amino acid sequence of the recombinant factor VIII polypeptide comprises at least a substitution or substitution set at amino acid position(s) 1051, 1221, 1218, 387, 749/1218, 332, 897, 696, 816, 824, 826, 393, 825, 698, 332/387/390/1148, 328/387/749, 749, 306/328/332/390/1051/1193/1218, 63/306/332/1051/1148, 63/306/332/387/1051, 387/390/1051, 186/306/328/332/390/749/1051/1148/1193, 387/390/756/1051, 306/390/1051, 306/332/390/749, 306/387/390/1148, 306/328/332/387/390/749/1051/1148/1193, 332/387/390/749/1148, 387/390/749, 306/387/749/1051/1148, 387/749, 306/945/1051/1193/1221, 306/390/749/1051, 63/306/332/390, 306/387/390/749/1148/1193/1221, 306/332/387/390/749/1218, 63/306/332/390/1051/1148/1193/1218, 924, 755, 752, 751, 923, 797, 754, 793, 753, 922, 896, 938, 775, 732, 810, 747, 393/407, 819, 787, 802, 811, 392, or 397, wherein the amino acid positions are relative to the reference sequence corresponding to SEQ ID NO: 706.

In some embodiments, the recombinant factor VIII polypeptide comprises an amino acid sequence having at least a substitution or substitution set 1051K, 1221G, 1218G, 387R, 1051R, 749F/1218G, 387T, 332P, 897K, 696E, 816W, 824W, 826G, 393W, 825L, 897R, 816V, 698W, 393Q, 332P/387R/390A/1148N, 328S/387R/749F, 749F, 306G/328S/332P/390A/1051R/1193A/1218G, 63Q/306G/332P/1051R/1148N, 63Q/306G/332P/387T/1051K, 387T/390A/1051R, 186A/306G/328S/332P/390A/749F/1051K/1148N/1193A, 387T/390A/756P/1051R, 306G/390A/1051R, 306G/332P/390A/749F, 306G/387R/390A/1148N, 306G/328S/332P/387T/390A/749F/1051R/1148N/1193A, 332P/387R/390A/749F/1148N, 387R/390A/749F, 306G/387T/749F/1051K/1148N, 387R/749F, 306G/945K/1051R/1193A/1221G, 306G/390A/749F/1051K, 63Q/306G/332P/390A, 306G/387T/390A/749F/1148N/1193A/1221G, 306G/332P/387R/390A/749F/1218M, 63Q/306G/332P/390A/1051R/1148N/1193A/1218G, 924I, 755S, 752F, 751Y, 923M, 797Y, 754W, 793G, 753D, 922W, 896L, 938R, 775G, 751E, 732W, 752G, 810V, 938V, 747D, 755G, 393G/407V, 797W, 819V, 393G, 787W, 393R, 802V, 775E, 924P, 732G, 811F, 802S, 752L, 392A, or 397G, wherein the amino acid positions are relative to the reference sequence corresponding to SEQ ID NO: 706.

In some embodiments, the amino acid sequence of the recombinant factor VIII polypeptide comprises at least a substitution or substitution set M1051K, L1221G, F1218G, I387R, M1051R, L749F/F1218G, I387T, S332P, M897K, T696E, F816W, F824W, A826G, V393W, I825L, M897R, F816V, F698W, V393Q, S332P/I387R/I390A/T1148N, F328S/I387R/L749F, L749F, E306G/F328S/S332P/I390A/M1051R/F1193A/F1218G, V63Q/E306G/S332P/M1051R/T1148N, V63Q/E306G/S332P/I387T/M1051K, I387T/I390A/M1051R, D186A/E306G/F328S/S332P/I390A/L749F/M1051K/T1148N/F1193A, I387T/I390A/I756P/M1051R, E306G/I390A/M1051R, E306G/S332P/I390A/L749F, E306G/I387R/I390A/T1148N, E306G/F328S/S332P/I387T/I390A/L749F/M1051R/T1148N/F1193A, S332P/I387R/I390A/L749F/T1148N, I387R/I390A/L749F, E306G/I387T/L749F/M1051K/T1148N, I387R/L749F, E306G/Q945K/M1051R/F1193A/L1221G, E306G/I390A/L749F/M1051K, V63Q/E306G/S332P/I390A, E306G/I387T/I390A/L749F/T1148N/F1193A/L1221G, E306G/S332P/I387R/I390A/L749F/F1218M, V63Q/E306G/S332P/I390A/M1051R/T1148N/F1193A/F1218G, G924I, A755S, K752F, S751Y, Q923M, M797Y, N754W, I793G, N753D, R922W, I896L, K938R, I775G, S751E, K732W, K752G, N810V, K938V, A747D, A755G, V393G/A407V, M797W, K819V, V393G, I787W, V393R, F802V, I775E, G924P, K732G, Q811F, F802S, K752L, S392A, or H397G, wherein the amino acid positions are relative to the reference sequence corresponding to SEQ ID NO: 706.

In some embodiments, the recombinant factor VIII polypeptide comprises an amino acid sequence having at least 70%, 75%, 80%, 81%, 82%, 83%, 84%, 85%, 86%, 87%, 88%, 89%, 90%, 91%, 92%, 93%, 94%, 95%, 96%, 97%, 98%, or 99% or more sequence identity to the reference sequence corresponding to residues 20-1457 of SEQ ID NO: 840, or to the reference sequence corresponding to SEQ ID NO: 840, wherein the amino acid sequence comprises one or more substitutions relative to the reference sequence corresponding to residues 20-1457 of SEQ ID NOs: 840, or relative to the reference sequence corresponding to SEQ ID NO: 840.

In some embodiments, the amino acid sequence of the recombinant factor VIII polypeptide comprises at least a substitution or substitution set at amino acid position(s) 620, 491, 1229, 615, 1283, 822, 328, 212, 818, 555/684, 828, 550, 830, 839, 13, 14, 17, 836, 1210, 1281, 64, 682, 318, 20, 21, 325, 533, 19, 817, 831, 225, 1156, 393/747, 753, 393/732/751, 63/797, 622, 617, 907, 1040, 611, 488, 1192, 1194, 619, 16, 802, 211, 555, 536, 527, 841, 320, 837, 558, 914, 312, 681, 838, 15, 1427, 18, 683, 24, 534, 840, 1160, 551, 393, 393/747/752/811/819/923/1148, 393/751/775, 63/393/732/775/1148, 393/732/1148, 775/1148, 393/752/753/775/793/797, 393/923, 393/923/1148, 393/747/751, 393/747/753/775/1148, 752/923/1051, 63/775, 63/393/752/775/793/1051/1053, 752/753/775/793/819, 63/1051, 63/747/752/753/775/811/819/923/1051, 393/752/787, 751, 63/1148, 63/747/752/753/819/923/1148, 1148, 747/752/753/793/811, 63, 797/1148, 751/752/753/1051, 63/732/1051, 752/797/815/923/1051/1148, or 747/753, wherein the amino acid positions are relative to the reference sequence corresponding to SEQ ID NO: 840.

In some embodiments, the amino acid sequence of the recombinant factor VIII polypeptide comprises at least a substitution or substitution set 620N, 491E, 1229D, 615N, 1283S, 822S, 328G, 212L, 818W, 1283L, 555G/684G, 828K, 550G, 830M, 839A, 822T, 13Y, 14G, 17V, 836V, 839P, 1210W, 1281R, 64D, 212G, 682R, 14M, 318C, 17S, 20L, 818G, 836G, 21M, 836R, 325Y, 533V, 19G, 14V, 817D, 831W, 225R, 1156R, 830V, 393G/747D, 753D, 393G/732W/751Y, 63Q/797W, 622D, 617E, 617N, 907D, 1040E, 617D, 611E, 620P, 617G, 488G, 1192D, 1194P, 619E, 615D, 16A, 802S, 818V, 211K, 836W, 555R, 536A, 527A, 555E, 841W, 320Q, 837G, 558R, 555G, 211R, 64G, 320R, 533Y, 914V, 16V, 1283E, 536M, 312L, 681N, 838T, 536R, 15G, 536P, 558W, 558E, 1427Y, 839G, 18L, 13G, 1210M, 1156G, 558L, 839K, 211P, 211G, 211Q, 16D, 320G, 683W, 24V, 534I, 681A, 840G, 1160R, 681C, 328Q, 15S, 536K, 536G, 533R, 681E, 536W, 1156N, 16P, 817A, 18R, 551W, 555T, 681G, 212A, 16G, 838G, 681R, 393G, 393G/747D/752G/811F/819V/923M/1148N, 393G/751Y/775G, 63Q/393G/732W/775G/1148N, 393G/732W/1148N, 775G/1148N, 393G/752L/753D/775G/793G/797W, 393G/923M, 393G/923M/1148N, 393G/747D/751Y, 393G/747D/753D/775G/1148N, 752L/923M/1051R, 63Q/775G, 63Q/393G/752L/775G/793G/1051R/1053R, 752G/753D/775G/793G/819V, 63Q/1051R, 63Q/747D/752G/753D/775G/811F/819V/923M/1051R, 393G/752G/787W, 751Y, 63Q/1148N, 63H/747D/752G/753D/819V/923M/1148N, 1148N, 747D/752L/753D/793G/811F, 63Q, 797W/1148N, 751Y/752L/753D/1051R, 63Q/732W/1051R, 752G/797W/815I/923M/1051R/1148N, or 747D/753D, wherein the amino acid positions are relative to the reference sequence corresponding to SEQ ID NO: 840.

In some embodiments, the amino acid sequence of the recombinant factor VIII polypeptide comprises at least a substitution or substitution set V620N, P491E, M1229D, P615N, I1283S, H822S, F328G, H212L, K818W, I1283L, F555G/E684G, V828K, R550G, R830M, S839A, H822T, L13Y, L14G, C17V, M836V, S839P, K1210W, Y1281R, V64D, H212G, V682R, L14M, L318C, C17S, A20L, K818G, M836G, T21M, M836R, F325Y, T533V, S19G, L14V, Q817D, L831W, K225R, H1156R, R830V, V393G/A747D, N753D, V393G/K732W/S751Y, V63Q/M797W, L622D, P617E, P617N, P907D, N1040E, P617D, Q611E, V620P, P617G, A488G, P1192D, S1194P, G619E, P615D, F16A, F802S, K818V, L211K, M836W, F555R, V536A, I527A, F555E, H841W, M320Q, S837G, M558R, F555G, L211R, V64G, M320R, T533Y, L914V, F16V, I1283E, V536M, F312L, M681N, S838T, V536R, R15G, V536P, M558W, M558E, L1427Y, S839G, F18L, L13G, K1210M, H1156G, M558L, S839K, L211P, L211G, L211Q, F16D, M320G, Y683W, Y24V, V534I, M681A, P840G, F1160R, M681C, F328Q, R15S, V536K, V536G, T533R, M681E, V536W, H1156N, F16P, Q817A, F18R, Y551W, F555T, M681G, H212A, F16G, S838G, M681R, V393G, V393G/A747D/K752G/Q811F/K819V/Q923M/T1148N, V393G/S751Y/I775G, V63Q/V393G/K732W/I775G/T1148N, V393G/K732W/T1148N, I775G/T1148N, V393G/K752L/N753D/I775G/I793G/M797W, V393G/Q923M, V393G/Q923M/T1148N, V393G/A747D/S751Y, V393G/A747D/N753D/I775G/T1148N, K752L/Q923M/M1051R, V63Q/I775G, V63Q/V393G/K752L/I775G/I793G/M1051R/T1053R, K752G/N753D/I775G/I793G/K819V, V63Q/M1051R, V63Q/A747D/K752G/N753D/I775G/Q811F/K819V/Q923M/M1051R, V393G/K752G/I787W, S751Y, V63Q/T1148N, V63H/A747D/K752G/N753D/K819V/Q923M/T1148N, T1148N, A747D/K752L/N753D/I793G/Q811F, V63Q, M797W/T1148N, S751Y/K752L/N753D/M1051R, V63Q/K732W/M1051R, K752G/M797W/S815I/Q923M/M1051R/T1148N, or A747D/N753D, wherein the amino acid positions are relative to the reference sequence corresponding to SEQ ID NO: 840.

In some embodiments, the recombinant factor VIII polypeptide comprises an amino acid sequence having at least 70%, 75%, 80%, 81%, 82%, 83%, 84%, 85%, 86%, 87%, 88%, 89%, 90%, 91%, 92%, 93%, 94%, 95%, 96%, 97%, 98%, or 99% or more sequence identity to the reference sequence corresponding to residues 20-1457 of SEQ ID NO: 1200, or to the reference sequence corresponding to SEQ ID NO: 1200, wherein the amino acid sequence comprises one or more substitutions relative to the reference sequence corresponding to residues 20-1457 of SEQ ID NOs: 1200, or relative to the reference sequence corresponding to SEQ ID NO: 1200.

In some embodiments, the amino acid sequence of the recombinant factor VIII polypeptide comprises at least a substitution or substitution set at amino acid position(s) 681, 622, 617, 558, 536, 211, 320, 839, 797, 533, 751, 303, 951, 164, 818, 807, 799, 735, 794, 926, 755, 921, 801, 1094, 968, 929, 924, 698, 168, 581, 916, 1428, 805, 1114, 311, 737, 693, 809, 757, 1099, 676, 740, 791, 211/536/622/681, 536/558, 211/536/555/617/732, 211/681/732, 211/536/555, 555, 211/536, 211/212, 211/536/617/681/732, 211/536/558/617/681, 555/732, 536/555/558/617/732, 536/617/681/732, 536/555, 536/622/681, 732, 212/320/533/617/681, 211/312/533/534/617/619/681/839, 212, 211/212/320/619/681/839, 797/839, 212/312/320/533/534/617/681, 558/619/681, 320/558/681, 212/320/534/619/681/797/839, 320/558/619/732/839, 211/212/312/534/558/619/681/732/839, 320/534/558/619/732/797, 558/617/619/681, 211/534/558/611/681, 211/320/617/619/681, 558/617/681/797, 320/534/619/732, 211/619/681, 211/212/534/558/619/732/839, 212/320/619/681/797/839, 212/320/611/839, 320/558/619/681, 312/533/617/681/797/839, 558/611/681, 312, 211/320/558/619/681/732/797/839, 320/558/732/797, 533/558/611/619/716/732/839, 312/681, 320/534/619/681, 211/320/533/536/619, 558/617/681/839, 212/320/534/558/681, 533/534/681/839, 212/534/619/681, 617/619/681, 211/212/320/558/617/681, 212/534/558/681/797, 212/320/534, 211/312/619, 212/312/619/839, 210/212/320/558/681, 212/312/534/681/797/839, 211/320/558/839, 294, 759, 538, or 1109, wherein the amino acid positions are relative to the reference sequence corresponding to SEQ ID NO: 1200.

In some embodiments, the amino acid sequence of the recombinant factor VIII polypeptide comprises at least a substitution or substitution set 681A, 622D, 617G, 558L, 536K, 681G, 211Q, 320R, 558W, 839K, 211K, 797W, 533R, 751A, 303T, 951V, 164E, 818G, 807L, 799A, 735S, 794R, 926V, 755P, 921W, 801A, 1094S, 968A, 929Q, 924W, 807P, 698D, 168G, 929V, 818A, 818S, 581K, 818P, 916K, 698S, 303G, 916C, 1428G, 805L, 924M, 1114M, 311G, 737N, 805V, 801I, 751D, 693T, 818E, 929A, 311E, 737L, 809W, 757A, 1099G, 799G, 916I, 805G, 737F, 737M, 799P, 807C, 916E, 676Q, 581V, 968P, 755Y, 916Q, 735D, 740G, 698Y, 757G, 791T, 1114A, 735V, 536P, 211R/536K/622D/681A, 536K/558L, 211R/536R/555G/617G/732K, 211R/681R/732K, 211R/536K/555E, 536R, 555E, 211R/536P, 211R/212P, 211R/536K, 211R/536K/617G/681A/732K, 211R, 211R/536P/558L/617G/681A, 555G/732K, 536P/555E/558L/617G/732K, 536K/617G/681A/732K, 536K/555G, 536P/622D/681A, 732K, 212A/320Q/533R/617E/681G, 211K/312L/533R/534I/617D/619E/681N/839K, 212A, 211K/212A/320Q/619E/681G/839K, 797W/S839K, 212A/312L/320Q/533R/534I/617E/681G, 558W/619E/681G, 320Q/558W/681G, 212A/320Q/534I/619E/681G/797W/839K, 320R/558W/619E/732K/839K, 211K/212A/312L/534I/558W/619E/681N/732K/839K, 320R/534I/558R/619E/732K/797W, 558W/617D/619E/681N, 211Q/534I/558W/611E/681N, 211K/320R/617E/619E/681G, 558W/617D/681G/797W, 320R/534I/619E/732K, 211K/619E/681N, 211K/212A/534I/558W/619E/732K/839K, 212A/320R/619E/681N/797W/839K, 212A/320R/611E/839K, 320Q/558W/619E/681N, 312L/533R/617D/681N/797W/839K, 558W/611E/681N, 312L, 211Q/320R/558W/619E/681G/732K/797W/839K, 320Q/558W/732K/797W, 533R/558W/611E/619E/716L/732K/839K, 312L/681G, 320Q/534I/619E/681N, 211K/320Q/533R/536L/619E, 558W/617E/681N/839K, 212A/320R/534I/558W/681G, 533R/534I/681N/839K, 212A/534I/619E/681G, 617E/619E/681G, 211Q/212A/320R/558W/617E/681N, 212A/534I/558W/681N/797W, 212A/320R/534I, 211K/312L/619E, 212A/312L/619E/839K, 210P/212A/320R/558W/681G, 212A/312L/534I/681N/797W/839K, 211K/320R/558W/839K, 924Q, 916L, 757T, 737K, 818V, 801G, 294G, 799S, 799E, 809G, 791M, 737V, 805Q, 799Q, 737S, 916M, 759S, 799V, 538V, 1109G, 168R, 921R, 926T, 916T, 737G, or 801R, wherein the amino acid positions are relative to the reference sequence corresponding to SEQ ID NO: 1200.

In some embodiments, the amino acid sequence of the recombinant factor VIII polypeptide comprises at least a substitution or substitution set M681A, L622D, P617G, M558L, V536K, M681G, L211Q, M320R, M558W, S839K, L211K, M797W, T533R, S751A, A303T, T951V, G164E, K818G, E807L, K799A, G735S, S794R, E926V, A755P, Q921W, D801A, E1094S, L968A, K929Q, G924W, E807P, F698D, S168G, K929V, K818A, K818S, R581K, K818P, S916K, F698S, A303G, S916C, T1428G, Y805L, G924M, L1114M, T311G, Y737N, Y805V, D801I, S751D, S693T, K818E, K929A, T311E, Y737L, E809W, E757A, A1099G, K799G, S916I, Y805G, Y737F, Y737M, K799P, E807C, S916E, T676Q, R581V, L968P, A755Y, S916Q, G735D, D740G, F698Y, E757G, D791T, L1114A, G735V, V536P, L211R/V536K/L622D/M681A, V536K/M558L, L211R/V536R/F555G/P617G/W732K, L211R/M681R/W732K, L211R/V536K/F555E, V536R, F555E, L211R/V536P, L211R/H212P, L211R/V536K, L211R/V536K/P617G/M681A/W732K, L211R, L211R/V536P/M558L/P617G/M681A, F555G/W732K, V536P/F555E/M558L/P617G/W732K, V536K/P617G/M681A/W732K, V536K/F555G, V536P/L622D/M681A, W732K, H212A/M320Q/T533R/P617E/M681G, L211K/F312L/T533R/V534I/P617D/G619E/M681N/S839K, H212A, L211K/H212A/M320Q/G619E/M681G/S839K, M797W/S839K, H212A/F312L/M320Q/T533R/V534I/P617E/M681G, M558W/G619E/M681G, M320Q/M558W/M681G, H212A/M320Q/V534I/G619E/M681G/M797W/S839K, M320R/M558W/G619E/W732K/S839K, L211K/H212A/F312L/V534I/M558W/G619E/M681N/W732K/S839K, M320R/V534I/M558R/G619E/W732K/M797W, M558W/P617D/G619E/M681N, L211Q/V534I/M558W/Q611E/M681N, L211K/M320R/P617E/G619E/M681G, M558W/P617D/M681G/M797W, M320R/V534I/G619E/W732K, L211K/G619E/M681N, L211K/H212A/V534I/M558W/G619E/W732K/S839K, H212A/M320R/G619E/M681N/M797W/S839K, H212A/M320R/Q611E/S839K, M320Q/M558W/G619E/M681N, F312L/T533R/P617D/M681N/M797W/S839K, M558W/Q611E/M681N, F312L, L211Q/M320R/M558W/G619E/M681G/W732K/M797W/S839K, M320Q/M558W/W732K/M797W, T533R/M558W/Q611E/G619E/F716L/W732K/S839K, F312L/M681G, M320Q/V534I/G619E/M681N, L211K/M320Q/T533R/V536L/G619E, M558W/P617E/M681N/S839K, H212A/M320R/V534I/M558W/M681G, T533R/V534I/M681N/S839K, H212A/V534I/G619E/M681G, P617E/G619E/M681G, L211Q/H212A/M320R/M558W/P617E/M681N, H212A/V534I/M558W/M681N/M797W, H212A/M320R/V534I, L211K/F312L/G619E, H212A/F312L/G619E/S839K, T210P/H212A/M320R/M558W/M681G, H212A/F312L/V534I/M681N/M797W/S839K, L211K/M320R/M558W/S839K, G924Q, S916L, E757T, Y737K, K818V, D801G, T294G, K799S, K799E, E809G, D791M, Y737V, Y805Q, K799Q, Y737S, S916M, R759S, K799V, D538V, E1109G, S168R, Q921R, E926T, S916T, Y737G, or D801R, wherein the amino acid positions are relative to the reference sequence of SEQ ID NO: 1200.

In some embodiments, the recombinant factor VIII polypeptide comprises an amino acid sequence having at least 70%, 75%, 80%, 81%, 82%, 83%, 84%, 85%, 86%, 87%, 88%, 89%, 90%, 91%, 92%, 93%, 94%, 95%, 96%, 97%, 98%, or 99% or more sequence identity to the reference sequence corresponding to residues 20-1457 of SEQ ID NO: 1514, or to the reference sequence corresponding to SEQ ID NO: 1514, wherein the amino acid sequence comprises one or more substitutions relative to the reference sequence corresponding to residues 20-1457 of SEQ ID NOs: 1514, or relative to the reference sequence corresponding to SEQ ID NO: 1514.

In some embodiments, the amino acid sequence of the recombinant factor VIII polypeptide comprises at least a substitution or substitution set at amino acid position(s) 1042, 1041, 1051, 1283, 1281, 916, 187, 215, 178, 1050, 1210, 104, 615, 610, 536, 618, 621, 614, 110, 301, 1078, 812, 786, 127, 175, 1093, 1088, 650, 535, 806, 738, 700, 132, 1076, 1084, 498, 756, 1108, 746, 160, 543, 310, 584, 918, 1452, 790, 292, 800, 167, 892, 304, 1116, 752, 281, 917, 712, 1080, 806/1352, 1095, 744, 1098, 948, 119, 785, 1113, 667, 158, 580, 707, 731, 1100, 1089, 1092, 939, 1104, 736, 1297, 1127, 686, 1129, 1138, 786/821, 680, 1101, 808, 739, 308, 536/555/619/732/799/805, 555/619/801, 555/619/799, 619/799/801/805, 536/555/619/732/799, 799/805, 801, 246/619/799/801, 536/555/619, 619/839, 619/799/805, 536/801/805, 555/619/799/805, 619/801/805, 555/619, 536/619/801, 801/805, 536/619/799/805, 555/619/732/799/801/805, 749, 616, 611, 370/614, 1156, 1158, 555/681/737/799, 536/619/809/1109, 536/555/619/737/799/809/839/916/921, 916/1109, 536/619/681/799/916/1109, 555/619/681/807/1109, 555/619/681/737/799, 619/681/799/916/1109, 619/681, 555/921, 619/681/916, 555/619/681/799, 555, 732, 619/799, 619, 681, 555/916, 839, or 799, wherein the amino acid positions are relative to the reference sequence corresponding to SEQ ID NO: 1514.

In some embodiments, the amino acid sequence of the recombinant factor VIII polypeptide comprises at least a substitution or substitution set 1042E, 1041N, 1051H, 1283H, 1283T, 1281Q, 1281P, 916E, 1051W, 187C, 215E, 215P, 215S, 178C, 1050T, 1210T, 1281K, 1042A, 104S, 615D, 610S, 610K, 610T, 536P, 618Y, 621W, 618P, 614F, 110H, 301K, 1078S, 812G, 786C, 127V, 175G, 1093W, 1088G, 1093T, 650M, 301M, 535N, 806R, 738E, 700E, 132R, 1076G, 1084L, 498A, 786L, 738F, 535G, 806E, 756G, 1108S, 1093V, 746P, 160E, 160T, 178V, 543L, 310G, 700Q, 1093A, 584A, 918S, 1452L, 738P, 790W, 790I, 292D, 800M, 1108M, 1088S, 167G, 892T, 790P, 800R, 738K, 304G, 1116G, 178Q, 1116A, 752G, 1108W, 738D, 281A, 917G, 712M, 1080V, 806G/1352E, 1095G, 1093G, 744A, 543G, 1098G, 281S, 756T, 948S, 119V, 1116L, 785V, 1113F, 700A, 786D, 175A, 790M, 667I, 158G, 580S, 707H, 731I, 918M, 1100S, 1089S, 712Y, 175H, 1092G, 939E, 1104E, 800G, 736F, 543W, 1297A, 712L, 1127V, 939S, 178M, 731R, 686W, 543P, 736G, 1129P, 1138Q, 812V, 786D/821Q, 535C, 680R, 1116I, 1089F, 304E, 178W, 1104C, 744R, 738R, 1093S, 712S, 187G, 132P, 812I, 1080G, 800C, 1101G, 808K, 808S, 790A, 800V, 680L, 167R, 752E, 1093N, 119G, 739G, 1116R, 580Y, 304A, 712Q, 806S, 712G, 790G, 1108V, 731L, 1084N, 1127L, 308P, 1076S, 1093P, 1093Y, 739R, 739P, 310V, 712T, 167H, 1093R, 536P/555E/619E/732K/799S/805Q, 555E/619E/801G, 555E/619E/799S, 619E/799S/801G/805Q, 536P/555E/619E/732K/799S, 799S/805Q, 801G, 246D/619E/799S/801G, 536P/555E/619E, 619E/839K, 619E/799S/805Q, 536P/801G/805Q, 555E/619E/799S/805Q, 619E/801G/805Q, 555E/619E, 536P/619E/801G, 801G/805Q, 536P/619E/799S/805Q, 555E/619E/732K/799S/801G/805Q, 1281H, 749L, 614H, 616D, 621K, 1050E, 610H, 611D, 1042T, 616Q, 614N, 370A/614A, 1156D, 1210V, 614T, 1158N, 1281S, 618E, 555E/681N/737K/799Q, 536P/619E/809G/1109G, 536K/555G/619E/737K/799Q/809G/839K/916T/921R, 916T/1109G, 536R/619E/681N/799Q/916T/1109G, 555G/619E/681N/807R/1109G, 555E/619E/681N/737K/799Q, 619E/681N/799S/916T/1109G, 619E/681N, 555G/921R, 619E/681N/916T, 555G/619E/681N/799S, 555E, 732K, 619E/799S, 619E, 681N, 555G/916T, 839K, or 799Q, wherein the amino acid positions are relative to the reference sequence corresponding to SEQ ID NO: 1514.

In some embodiments, the amino acid sequence of the recombinant factor VIII polypeptide comprises at least a substitution or substitution set R1042E, Y1041N, M1051H, I1283H, I1283T, Y1281Q, Y1281P, S916E, M1051W, L187C, I215E, I215P, I215S, L178C, I1050T, K1210T, Y1281K, R1042A, V104S, P615D, I610S, I610K, I610T, V536P, A618Y, Q621W, A618P, L614F, M110H, R301K, I1078S, S812G, E786C, A127V, Y175G, K1093W, F1088G, K1093T, L650M, R301M, T535N, D806R, Y738E, S700E, E132R, E1076G, S1084L, G498A, E786L, Y738F, T535G, D806E, I756G, F1108S, K1093V, S746P, L160E, L160T, L178V, S543L, 1310G, S700Q, K1093A, Q584A, E918S, E1452L, Y738P, D790W, D790I, G292D, E800M, F1108M, F1088S, A167G, V892T, D790P, E800R, Y738K, S304G, S1116G, L178Q, S1116A, K752G, F1108W, Y738D, T281A, Y917G, H712M, S1080V, D806G/K1352E, E1095G, K1093G, D744A, S543G, M1098G, T281S, I756T, M948S, A119V, S1116L, E785V, M1113F, S700A, E786D, Y175A, D790M, F667I, Q158G, Q580S, W707H, D731I, E918M, L1100S, T1089S, H712Y, Y175H, K1092G, T939E, Y1104E, E800G, D736F, S543W, N1297A, H712L, 11127V, T939S, L178M, D731R, T686W, S543P, D736G, E1129P, L1138Q, S812V, E786D/R821Q, T535C, K680R, S1116I, T1089F, S304E, L178W, Y1104C, D744R, Y738R, K1093S, H712S, L187G, E132P, S812I, S1080G, E800C, Y1101G, D808K, D808S, D790A, E800V, K680L, A167R, K752E, K1093N, A119G, E739G, S1116R, Q580Y, S304A, H712Q, D806S, H712G, D790G, F1108V, D731L, S1084N, I1127L, S308P, E1076S, K1093P, K1093Y, E739R, E739P, I310V, H712T, A167H, K1093R, V536P/F555E/G619E/W732K/K799S/Y805Q, F555E/G619E/D801G, F555E/G619E/K799S, G619E/K799S/D801G/Y805Q, V536P/F555E/G619E/W732K/K799S, K799S/Y805Q, D801G, A246D/G619E/K799S/D801G, V536P/F555E/G619E, G619E/S839K, G619E/K799S/Y805Q, V536P/D801G/Y805Q, F555E/G619E/K799S/Y805Q, G619E/D801G/Y805Q, F555E/G619E, V536P/G619E/D801G, D801G/Y805Q, V536P/G619E/K799S/Y805Q, F555E/G619E/W732K/K799S/D801G/Y805Q, Y1281H, F749L, L614H, N616D, Q621K, I1050E, I610H, Q611D, R1042T, N616Q, L614N, T370A/L614A, H1156D, K1210V, L614T, R1158N, Y1281S, A618E, F555E/G681N/Y737K/K799Q, V536P/G619E/E809G/E1109G, V536K/F555G/G619E/Y737K/K799Q/E809G/S839K/S916T/Q921R, S916T/E1109G, V536R/G619E/G681N/K799Q/S916T/E1109G, F555G/G619E/G681N/E807R/E1109G, F555E/G619E/G681N/Y737K/K799Q, G619E/G681N/K799S/S916T/E1109G, G619E/G681N, F555G/Q921R, G619E/G681N/S916T, F555G/G619E/G681N/K799S, F555E, W732K, G619E/K799S, G619E, G681N, F555G/S916T, S839K, or K799Q, wherein the amino acid positions are relative to the reference sequence corresponding to SEQ ID NO: 1514.

In some embodiments, the recombinant factor VIII polypeptide comprises an amino acid sequence having at least 70%, 75%, 80%, 81%, 82%, 83%, 84%, 85%, 86%, 87%, 88%, 89%, 90%, 91%, 92%, 93%, 94%, 95%, 96%, 97%, 98%, or 99% or more sequence identity to the reference sequence corresponding to residues 20-1457 of SEQ ID NO: 2072, or to the reference sequence corresponding to SEQ ID NO: 2072, wherein the amino acid sequence comprises one or more substitutions relative to the reference sequence corresponding to residues 20-1457 of SEQ ID NOs: 2072, or relative to the reference sequence corresponding to SEQ ID NO: 2072.

In some embodiments, the amino acid sequence of the recombinant factor VIII polypeptide comprises at least a substitution or substitution set at amino acid position(s) 167, 167/738/1076/1093, 167/738/752/800/805/808/809/1116, 167/800/809/1076, 167/318, 167/318/800/805/808/809, 167/318/681, 167/318/681/700/805/808/809/1076/1116, 167/318/700, 167, 167/738/1076/1093, 167/738/752/800/805/808/809/1116, 167/800/809/1076, 167/318, 167/318/800/805/808/809, 167/318/681, 167/318/681/700/805/808/809/1076/1116, 167/318/700, 167/318/700/1076, 167/318/700/738/739/1076, 167/681/738/800/808/809, 167/178/700/752/1093, 167/700, 167/700/738/739/1093, 167/700/738/752/1093, 167/700/738/752/805/808/809/1093/1116, 808/809, 1076, 738, 738/808/809, 738/1076, 738/739, 738/739/809, 738/739/809/1093/1116, 738/739/1093, 738/739/752/1093, 738/739/799/800/1093, 738/739/1116, 738/800, 738/1093, 738/752, 738/752/1093, 738/752/799, 738/799/809/1093, 738/799/1093, 739, 739/800/809, 800, 800/805/809, 809, 809/1093, 809/1093/1116, 809/1116, 318, 318/1076, 318/1076/1116, 318/739/805/808/809/1076, 318/499/700/738/809, 318/1093, 318/752/1093, 318/1116, 318/543/739, 318/543/681/752/1116, 318/543/1093, 318/700/738/1093/1414, 681, 681/738, 681/738/739/1093, 681/738/739/752/799/800/809/1116/1339, 681/738/739/1116, 681/738/800, 681/800/805/809, 681/809/1093, 681/752/1093, 681/752/1093, 681/700, 681/700/738/739/1093/1116, 681/700/809, 681/700/1093, 681/700/752/808/809/1093/1116, 681/700/752/1093, 681/700/844, 681/700/805/809, 1093, 1093/1116, 752, 752/1076, 752/800/809, 752/800/1093, 752/809, 752/866, 752/1093, 752/1116, 178/318/739/752/809/1076/1116, 178/318/800/809, 178/318/681/800/808/809/1076, 178/681, 178/681/738/1116, 178/752/809, 178/700/800/805/808/809, 178/805/809/866, 1116, 543, 543/738/739/809, 543/738/809/1093, 543/739/1093, 543/800/1093/1116, 543/809/1093, 543/809/1093/1116, 543/681/738/739/752/809/1093, 543/681/738/739/752/1093, 543/681/739/752/800/805/809/1093, 543/681/700/738/739/800/1093/1116, 543/681/700/738/739/805/808/809, 543/681/700/752, 543/681/700/752/809/1093, 543/1093, 543/700, 543/700/738/739/752/800/1093/1116, 543/700/809, 543/700/1093, 543/700/752, 543/700/752/800/1093, 543/700/752/809/1116, 543/700/752/1093, 700, 700/738/739/800/1093, 700/738/739/799/1093, 700/738/800/1093, 700/738/1116, 700/809, 700/809/1093, 700/1093, 700/1093/1116, 700/752, 700/752/808/809/1093/1116, 700/752/809/1093, 700/752/799/1116, 700/752/805/809/1093, 700/799/800, 700/1116, 700/805/808/809/1076, 514/543/681/805/808/809, 805/808/809, or 805/809/1116, wherein the amino acid positions are relative to the reference sequence corresponding to SEQ ID NO: 2072.

In some embodiments, the amino acid sequence of the recombinant factor VIII polypeptide comprises at least a substitution or substitution set 167G, 167G/738Y/1076S/1093G, 167G/738Y/752E/800V/805Q/808S/809G/1116I, 167G/800V/809G/1076S, 167G/318L, 167G/318L/800V/805Q/808S/809G, 167G/318L/681N, 167G/318L/681N/700E/805Q/808S/809G/1076S/1116I, 167G/318L/700E, 167G/318L/700E/1076S, 167G/318L/700E/738Y/739G/1076S, 167G/681N/738Y/800V/808S/809G, 167G/178Q/700E/752E/1093G, 167G/700E, 167G/700E/738Y/739G/1093G, 167G/700E/738Y/752E/1093G, 167G/700E/738Y/752E/805Q/808S/809G/1093A/1116I, 808S/809G, 1076S, 738Y, 738Y/808S/809G, 738Y/1076S, 738Y/739G, 738Y/739G/809G, 738Y/739G/809G/1093G/1116I, 738Y/739G/1093G, 738Y/739G/752E/1093G, 738Y/739G/799S/800V/1093G, 738Y/739G/1116I, 738Y/800V, 738Y/1093G, 738Y/752E, 738Y/752E/1093G, 738Y/752E/799S, 738Y/799S/809G/1093G, 738Y/799S/1093G, 739G, 739G/800V/809G, 800V, 800V/805Q/809G, 809G, 809G/1093G, 809G/1093G/1116I, 809G/1116I, 318L, 318L/1076S, 318L/1076S/1116I, 318L/739G/805Q/808S/809G/1076S, 318L/499L/700E/738Y/809G, 318L/1093A, 318L/752E/1093A, 318L/1116I, 318L/543P/739G, 318L/543P/681N/752E/1116I, 318L/543P/1093A, 318L/700E/738Y/1093A/1414N, 681N, 681N/738Y, 681N/738Y/739G/1093G, 681N/738Y/739G/752E/799S/800V/809G/1116I/1339C, 681N/738Y/739G/1116I, 681N/738Y/800V, 681N/800V/805Q/809G, 681N/809G/1093G, 681N/752E/1093A, 681N/752E/1093G, 681N/700E, 681N/700E/738Y/739G/1093G/1116I, 681N/700E/809G, 681N/700E/1093G, 681N/700E/752E/808S/809G/1093G/1116I, 681N/700E/752E/1093G, 681N/700E/844H, 681N/700E/805Q/809G, 1093A, 1093G, 1093G/1116I, 752E, 752E/1076S, 752E/800V/809G, 752E/800V/1093G, 752E/809G, 752E/866A, 752E/1093G, 752E/1116I, 178Q/318L/739G/752E/809G/1076S/1116I, 178Q/318L/800V/809G, 178Q/318L/681N/800V/808S/809G/1076S, 178Q/681N, 178Q/681N/738Y/1116I, 178Q/752E/809G, 178Q/700E/800V/805Q/808S/809G, 178Q/805Q/809G/866A, 1116I, 543P, 543P/738Y/739G/809G, 543P/738Y/809G/1093G, 543P/739G/1093A, 543P/800V/1093G/1116I, 543P/809G/1093G, 543P/809G/1093G/1116I, 543P/681N/738Y/739G/752E/809G/1093G, 543P/681N/738Y/739G/752E/1093G, 543P/681N/739G/752E/800V/805Q/809G/1093G, 543P/681N/700E/738Y/739G/800V/1093G/1116I, 543P/681N/700E/738Y/739G/805Q/808S/809G, 543P/681N/700E/752E, 543P/681N/700E/752E/809G/1093G, 543P/1093G, 543P/700E, 543P/700E/738Y/739G/752E/800V/1093G/1116I, 543P/700E/809G, 543P/700E/1093G, 543P/700E/752E, 543P/700E/752E/800V/1093G, 543P/700E/752E/809G/1116I, 543P/700E/752E/1093G, 700E, 700E/738Y/739G/800V/1093G, 700E/738Y/739G/799S/1093G, 700E/738Y/800V/1093G, 700E/738Y/1116I, 700E/809G, 700E/809G/1093G, 700E/1093G, 700E/1093G/1116I, 700E/752E, 700E/752E/808S/809G/1093G/1116I, 700E/752E/809G/1093G, 700E/752E/799S/1116I, 700E/752E/805Q/809G/1093A, 700E/799S/800V, 700E/1116I, 700E/805Q/808S/809G/1076S, 514L/543P/681N/805Q/808S/809G, 805Q/808S/809G, or 805Q/809G/1116I, wherein the amino acid positions are relative to the reference sequence corresponding to SEQ ID NO: 2072.

In some embodiments, the amino acid sequence of the recombinant factor VIII polypeptide comprises at least a substitution or substitution set A167G, A167G/E738Y/E1076S/K1093G, A167G/E738Y/K752E/E800V/Y805Q/D808S/E809G/S1116I, A167G/E800V/E809G/E1076S, A167G/G318L, A167G/G318L/E800V/Y805Q/D808S/E809G, A167G/G318L/G681N, A167G/G318L/G681N/S700E/Y805Q/D808S/E809G/E1076S/S1116I, A167G/G318L/S700E, A167G/G318L/S700E/E1076S, A167G/G318L/S700E/E738Y/E739G/E1076S, A167G/G681N/E738Y/E800V/D808S/E809G, A167G/L178Q/S700E/K752E/K1093G, A167G/S700E, A167G/S700E/E738Y/E739G/K1093G, A167G/S700E/E738Y/K752E/K1093G, A167G/S700E/E738Y/K752E/Y805Q/D808S/E809G/K1093A/S1116I, D808S/E809G, E1076S, E738Y, E738Y/D808S/E809G, E738Y/E1076S, E738Y/E739G, E738Y/E739G/E809G, E738Y/E739G/E809G/K1093G/S1116I, E738Y/E739G/K1093G, E738Y/E739G/K752E/K1093G, E738Y/E739G/K799S/E800V/K1093G, E738Y/E739G/S1116I, E738Y/E800V, E738Y/K1093G, E738Y/K752E, E738Y/K752E/K1093G, E738Y/K752E/K799S, E738Y/K799S/E809G/K1093G, E738Y/K799S/K1093G, E739G, E739G/E800V/E809G, E800V, E800V/Y805Q/E809G, E809G, E809G/K1093G, E809G/K1093G/S1116I, E809G/S1116I, G318L, G318L/E1076S, G318L/E1076S/S1116I, G318L/E739G/Y805Q/D808S/E809G/E1076S, G318L/I499L/S700E/E738Y/E809G, G318L/K1093A, G318L/K752E/K1093A, G318L/S1116I, G318L/S543P/E739G, G318L/S543P/G681N/K752E/S1116I, G318L/S543P/K1093A, G318L/S700E/E738Y/K1093A/S1414N, G681N, G681N/E738Y, G681N/E738Y/E739G/K1093G, G681N/E738Y/E739G/K752E/K799S/E800V/E809G/S1116I/G1339C, G681N/E738Y/E739G/S1116I, G681N/E738Y/E800V, G681N/E800V/Y805Q/E809G, G681N/E809G/K1093G, G681N/K752E/K1093A, G681N/K752E/K1093G, G681N/S700E, G681N/S700E/E738Y/E739G/K1093G/S1116I, G681N/S700E/E809G, G681N/S700E/K1093G, G681N/S700E/K752E/D808S/E809G/K1093G/S1116I, G681N/S700E/K752E/K1093G, G681N/S700E/R844H, G681N/S700E/Y805Q/E809G, K1093A, K1093G, K1093G/S1116I, K752E, K752E/E1076S, K752E/E800V/E809G, K752E/E800V/K1093G, K752E/E809G, K752E/G866A, K752E/K1093G, K752E/S1116I, L178Q/G318L/E739G/K752E/E809G/E1076S/S1116I, L178Q/G318L/E800V/E809G, L178Q/G318L/G681N/E800V/D808S/E809G/E1076S, L178Q/G681N, L178Q/G681N/E738Y/S1116I, L178Q/K752E/E809G, L178Q/S700E/E800V/Y805Q/D808S/E809G, L178Q/Y805Q/E809G/G866A, S1116I, S543P, S543P/E738Y/E739G/E809G, S543P/E738Y/E809G/K1093G, S543P/E739G/K1093A, S543P/E800V/K1093G/S1116I, S543P/E809G/K1093G, S543P/E809G/K1093G/S1116I, S543P/G681N/E738Y/E739G/K752E/E809G/K1093G, S543P/G681N/E738Y/E739G/K752E/K1093G, S543P/G681N/E739G/K752E/E800V/Y805Q/E809G/K1093G, S543P/G681N/S700E/E738Y/E739G/E800V/K1093G/S1116I, S543P/G681N/S700E/E738Y/E739G/Y805Q/D808S/E809G, S543P/G681N/S700E/K752E, S543P/G681N/S700E/K752E/E809G/K1093G, S543P/K1093G, S543P/S700E, S543P/S700E/E738Y/E739G/K752E/E800V/K1093G/S1116I, S543P/S700E/E809G, S543P/S700E/K1093G, S543P/S700E/K752E, S543P/S700E/K752E/E800V/K1093G, S543P/S700E/K752E/E809G/S1116I, S543P/S700E/K752E/K1093G, S700E, S700E/E738Y/E739G/E800V/K1093G, S700E/E738Y/E739G/K799S/K1093G, S700E/E738Y/E800V/K1093G, S700E/E738Y/S1116I, S700E/E809G, S700E/E809G/K1093G, S700E/K1093G, S700E/K1093G/S1116I, S700E/K752E, S700E/K752E/D808S/E809G/K1093G/S1116I, S700E/K752E/E809G/K1093G, S700E/K752E/K799S/S1116I, S700E/K752E/Y805Q/E809G/K1093A, S700E/K799S/E800V, S700E/S1116I, S700E/Y805Q/D808S/E809G/E1076S, V514L/S543P/G681N/Y805Q/D808S/E809G, Y805Q/D808S/E809G, or Y805Q/E809G/S1116I, wherein the amino acid positions are relative to the reference sequence corresponding to SEQ ID NO: 2072.

In some embodiments, the recombinant factor VIII polypeptide comprises an amino acid sequence comprising at least a substitution at an amino position set forth in Tables 4.1, 4.2, 5.1, 5.2, 6.1, 6.2, 7.1, 7.2, 8.1, 8.2, 9.1, 9.2, 10.1, 10.2, 11.1, and 15.1, wherein the amino acid positions are relative to the reference sequence corresponding to SEQ ID NO: 2, 252, 504, 706, 840, 1200, 1514, or 2072.

In some embodiments, the recombinant factor VIII polypeptide comprises an amino acid sequence comprising at least one substitution set forth in Tables 4.1, 4.2, 5.1, 5.2, 6.1, 6.2, 7.1, 7.2, 8.1, 8.2, 9.1, 9.2, 10.1, 10.2, 11.1, and 15.1, wherein the amino acid positions are relative to the reference sequence corresponding to SEQ ID NO: 2, 252, 504, 706, 840, 1200, 1514, or 2072.

In some embodiments, the recombinant factor VIII polypeptide comprises an amino acid sequence comprising at least a substitution or substitution set forth in Tables 4.1, 4.2, 5.1, 5.2, 6.1, 6.2, 7.1, 7.2, 8.1, 8.2, 9.1, 9.2, 10.1, 10.2, 11.1, and 15.1, wherein the amino acid positions are relative to the reference sequence corresponding to SEQ ID NO: 2, 252, 504, 706, 840, 1200, 1514, or 2072.

In some embodiments, the recombinant factor VIII polypeptide comprises an amino acid sequence comprising at least a substitution or substitution of a variant set forth in Tables 4.1, 4.2, 5.1, 5.2, 6.1, 6.2, 7.1, 7.2, 8.1, 8.2, 9.1, 9.2, 10.1, 10.2, 11.1, and 15.1, wherein the amino acid positions are relative to the reference sequence corresponding to SEQ ID NO: 2, 252, 504, 706, 840, 1200, 1514, or 2072.

In some embodiments, the recombinant factor VIII polypeptide comprises an amino acid sequence comprising residues 20-1457 of an even-numbered SEQ ID NO. of SEQ ID NOs: 4-2346 and 2360-2870, or an amino acid sequence comprising an even-numbered SEQ ID NO. of SEQ ID NOs: 4-2346 and 2350-2870, wherein optionally the amino acid sequence includes 1, 2, 3, 4, 5, 6, 7, 8, 9, or up to 10 amino acid substitutions, insertions or deletions. In some embodiments, the amino acid sequence optionally includes 1, 2, 3, 4, or 5 amino acid substitutions, insertions, or deletions. In some embodiments of the foregoing, the amino acid sequence optionally includes 1, 2, 3, 4, 5, 6, 7, 8, 9, or up to 10 amino acid substitutions, or 1, 2, 3, 4, or 5 substitutions. In some embodiments, the substitutions comprise non-conservative substitutions. In some embodiments, the substitutions comprise conservative substitutions.

In some embodiments, the recombinant factor VIII polypeptide comprises an amino acid sequence comprising residues 20-1457 of an even-numbered SEQ ID NO. of SEQ ID NOs: 4-316, or an amino acid sequence comprising an even-numbered SEQ ID NO. of SEQ ID NOs: 4-316. In some embodiments, the recombinant factor VIII polypeptide comprises an amino acid sequence comprising residues 20-1457 of an even-numbered SEQ ID NO. of SEQ ID NOs: 318-410 or 412-666, or an amino acid sequence comprising an even-numbered SEQ ID NO. of SEQ ID NOs: 318-410 or 412-666. In some embodiments, the recombinant factor VIII polypeptide comprises an amino acid sequence comprising residues 20-1457 of an even-numbered SEQ ID NO. of SEQ ID NOs: 668-700 or 702-780, or an amino acid sequence comprising an even-numbered SEQ ID NO. of SEQ ID NOs: 668-700 or 702-780. In some embodiments, the recombinant factor VIII polypeptide comprises an amino acid sequence comprising residues 20-1457 of an even-numbered SEQ ID NO. of SEQ ID NOs: 782-818 or 820-934, or an amino acid sequence comprising an even-numbered SEQ ID NO. of SEQ ID NOs: 782-818 or 820-934. In some embodiments, the recombinant factor VIII polypeptide comprises an amino acid sequence comprising residues 20-1457 of an even-numbered SEQ ID NO. of SEQ ID NOs: 936-1028 or 1030-1250, or an amino acid sequence comprising an even-numbered SEQ ID NO. of SEQ ID NOs: 936-1028 or 1030-1250. In some embodiments, the recombinant factor VIII polypeptide comprises an amino acid sequence comprising residues 20-1457 of an even-numbered SEQ ID NO. of SEQ ID NOs: 1252-1408 or 1410-1588, or an amino acid sequence comprising an even-numbered SEQ ID NO. of SEQ ID NOs: 1252-1408 or 1410-1588. In some embodiments, the recombinant factor VIII polypeptide comprises an amino acid sequence comprising residues 20-1457 of an even-numbered SEQ ID NO. of SEQ ID NOs: 1590-1648 or 1650-2068, or an amino acid sequence comprising an even-numbered SEQ ID NO. of SEQ ID NOs: 1590-1648 or 1650-2068. In some embodiments, the recombinant factor VIII polypeptide comprises an amino acid sequence comprising residues 20-1457 of an even-numbered SEQ ID NO. of SEQ ID NOs: 2070-2346, or an amino acid sequence comprising an even-numbered SEQ ID NO. of SEQ ID NOs: 2070-2346. In some embodiments, the recombinant factor VIII polypeptide comprises an amino acid sequence comprising residues 20-1457 of an even-numbered SEQ ID NO. of SEQ ID NOs: 2360-2870, or an amino acid sequence comprising an even-numbered SEQ ID NO. of SEQ ID NOs: 2350-2870. In some embodiments, the amino acid sequence optionally includes 1, 2, 3, 4, 5, 6, 7, 8, 9, or up to 10 amino acid substitutions, insertions or deletions. In some embodiments, the amino acid sequence of the recombinant factor VIII polypeptide optionally includes 1, 2, 3, 4, or 5 amino acid substitutions, insertions, or deletions. In some embodiments of the foregoing, the amino acid sequence optionally includes 1, 2, 3, 4, 5, 6, 7, 8, 9, or up to 10 amino acid substitutions, or 1, 2, 3, 4, or 5 amino acid substitutions. In some embodiments, the substitutions comprise non-conservative substitutions. In some embodiments, the substitutions comprise conservative substitutions.

In some embodiments, the recombinant factor VIII polypeptide comprises an amino acid sequence comprising residues 20-1457 of SEQ ID NO: 252, 504, 706, 840, 1200, 1514, or 2072, or an amino acid sequence comprising SEQ ID NO: 252, 504, 706, 840, 1200, 1514, or 2072. In some embodiments, the amino acid sequence optionally includes 1, 2, 3, 4, 5, 6, 7, 8, 9, or up to 10 amino acid substitutions, insertions or deletions. In some embodiments, optionally the amino acid sequence of the recombinant factor VIII polypeptide includes 1, 2, 3, 4, or 5 amino acid substitutions, insertions, or deletions. In some embodiments of the foregoing, optionally the amino acid sequence includes 1, 2, 3, 4, 5, 6, 7, 8, 9, or up to 10 amino acid substitutions, or 1, 2, 3, 4, or 5 amino acid substitutions. In some embodiments, the substitutions comprise non-conservative substitutions. In some embodiments, the substitutions comprise conservative substitutions.

In some embodiments, the recombinant factor VIII polypeptide described herein has clotting activity. In some embodiments, the recombinant factor VIII polypeptide has an improved property compared to a reference factor VIII polypeptide, wherein the reference factor VIII polypeptide has a sequence corresponding to residues 20-1457 of SEQ ID NO: 2, 252, 504, 706, 840, 1200, 1514, or 2072, or a sequence corresponding to SEQ ID NO: 2, 252, 504, 706, 840, 1200, 1514, or 2072.

In some embodiments, the recombinant factor VIII polypeptide has increased activity compared to the reference factor VIII polypeptide having a sequence corresponding to residues 20-1457 of SEQ ID NO: 2, 252, 504, 706, 840, 1200, 1514, or 2072, or a sequence corresponding to SEQ ID NO: 2, 252, 504, 706, 840, 1200, 1514, or 2072.

In some embodiments, the increased activity is clotting activity, particularly increased potency, compared to the reference factor VIII polypeptide having a sequence corresponding to residues 20-1457 of SEQ ID NO: 2, 252, 504, 706, 840, 1200, 1514, or 2072, or a sequence corresponding to SEQ ID NO: 2, 252, 504, 706, 840, 1200, 1514, or 2072.

In some embodiments, the recombinant factor VIII polypeptide has increased stability to serum and/or plasma exposure, particularly serum or plasma from a hemophilia A patient, compared to the reference factor VIII polypeptide having a sequence corresponding to residues 20-1457 of SEQ ID NO: 2, 252, 504, 706, 840, 1200, 1514, or 2072, or a sequence corresponding to SEQ ID NO: 2, 252, 504, 706, 840, 1200, 1514, or 2072.

In some embodiments, the recombinant factor VIII polypeptide is more stable at basic pH, e.g., about pH 7.4, than the reference factor VIII polypeptide having a sequence corresponding to residues 20-1457 of SEQ ID NO: 2, 252, 504, 706, 840, 1200, 1514, or 2072, or a sequence corresponding to SEQ ID NO: 2, 252, 504, 706, 840, 1200, 1514, or 2072.

In some embodiments, the recombinant factor VIII polypeptide has increased thermostability compared to the reference factor VIII polypeptide having a sequence corresponding to residues 20-1457 of SEQ ID NO: 2, 252, 504, 706, 840, 1200, 1514, or 2072, or a sequence corresponding to SEQ ID NO: 2, 252, 504, 706, 840, 1200, 1514, or 2072.

In some embodiments, the recombinant factor VIII polypeptide is characterized by increased expression and/or secretion from cells that are transfected with an expression vector as compared to a reference factor VIII polypeptide having a sequence corresponding to residues 20-1457 of SEQ ID NO: 2, 252, 504, 706, 840, 1200, 1514, or 2072, or a sequence corresponding to SEQ ID NO: 2, 252, 504, 706, 840, 1200, 1514, or 2072.

In some embodiments, the recombinant factor VIII polypeptide is characterized by increased transduction and/or secretion from cells that are transduced with a gene therapy composition (e.g., AAV, mRNA, or lipid nanoparticle) as compared to a reference factor VIII polypeptide having a sequence corresponding to residues 20-1457 of SEQ ID NO: 2, 252, 504, 706, 840, 1200, 1514, or 2072, or a sequence corresponding to SEQ ID NO: 2, 252, 504, 706, 840, 1200, 1514, or 2072.

In some embodiments, the improved property is in comparison to a reference factor VIII polypeptide having the sequence corresponding to residues 20-1457 of SEQ ID NO: 2, or the sequence corresponding to SEQ ID NO: 2.

In some embodiments, the recombinant factor VIII polypeptide of the present disclosure exhibits at least one improved property selected from: i) increased activity/potency; ii) increased clotting activity/potency; iii) increased stability to plasma or serum; iv) increased thermostability; v) reduced immunogenicity; vi) increased expression and/or secretion from cells that are transfected with an expression vector; vii) increased transduction and/or secretion from cells that are transduced with a gene therapy vector (e.g., AAV, mRNA, or lipid nanoparticle), or a combination of any of i), ii), iii), iv), v), vi, and/or vii), as compared to a reference factor VIII polypeptide having a sequence corresponding to residues 20-1457 of SEQ ID NO: 2, 252, 504, 706, 840, 1200, 1514, or 2072, or a sequence corresponding to SEQ ID NO: 2, 252, 504, 706, 840, 1200, 1514, or 2072. In some embodiments, the reference recombinant factor VIII polypeptide has the sequence corresponding to residues 20-1457 of SEQ ID NO: 2, or the sequence corresponding to SEQ ID NO: 2.

In some embodiments, the recombinant factor VIII polypeptide is a “deimmunized” recombinant factor VIII polypeptide (e.g., as presented in the Examples in Tables 12.1, 12.2, 12.3, 12.4, 12.5, 12.6, 12.7, 12.8, and 15.2). In some embodiments, the recombinant factor VIII polypeptide is characterized by a reduction of Total Immunogenicity Score (TIS) and/or Immunogenic Hit Count (IHC) as compared to a reference factor VIII polypeptide, wherein the reference factor VIII polypeptide has a sequence corresponding to SEQ ID NO: 2, 252, 504, 706, 840, 1200, 1514, or 2072, such as provided in Tables 12.1, 12.2, 12.3, 12.4, 12.5, 12.6, 12.7, 12.8, and 15.2 of the Examples.

In some embodiments, a “deimmunized” recombinant factor VIII polypeptide comprises an amino acid sequence comprising residues 20-1457 or residues 1-1457 of a sequence selected from SEQ ID NOs: 4, 6, 8, 10, 12, 14, 16, 18, 20, 26, 28, 32, 34, 36, 38, 40, 42, 44, 46, 48, 50, 52, 54, 56, 60, 64, 66, 68, 72, 74, 76, 78, 82, 84, 86, 88, 90, 92, 94, 96, 98, 100, 102, 104, 106, 108, 110, 112, 114, 116, 118, 120, 122, 130, 132, 134, 136, 138, 140, 142, 146, 148, 150, 152, 154, 156, 158, 160, 162, 164, 166, 168, 170, 172, 174, 176, 178, 180, 182, 184, 186, 188, 190, 192, 194, 196, 198, 200, 202, 204, 206, 208, 210, 212, 214, 216, 218, 220, 222, 224, 226, 228, 230, 232, 234, 236, 238, 242, 244, 248, 252, 260, 262, 266, 270, 274, 278, 280, 282, 284, 286, 288, 290, 292, 296, 298, 300, 302, 310, 312, 320, 322, 324, 326, 328, 330, 332, 334, 336, 338, 340, 342, 344, 346, 348, 350, 352, 354, 356, 358, 360, 362, 364, 366, 368, 370, 372, 374, 376, 378, 380, 382, 384, 386, 388, 390, 392, 394, 396, 398, 400, 402, 404, 406, 408, 410, 412, 414, 416, 418, 420, 424, 426, 428, 430, 432, 434, 436, 438, 442, 444, 446, 448, 452, 454, 456, 458, 460, 462, 464, 466, 468, 470, 472, 474, 476, 478, 480, 482, 484, 486, 488, 490, 492, 498, 500, 502, 504, 506, 508, 510, 512, 514, 516, 518, 520, 522, 524, 526, 530, 532, 534, 536, 538, 540, 542, 544, 546, 548, 550, 552, 554, 556, 558, 560, 562, 564, 566, 568, 570, 574, 578, 580, 582, 584, 586, 588, 590, 592, 594, 596, 598, 600, 602, 604, 606, 608, 610, 612, 614, 618, 620, 622, 624, 626, 628, 630, 632, 634, 636, 638, 640, 642, 644, 646, 648, 650, 652, 654, 656, 658, 660, 662, 664, 666, 668, 670, 672, 674, 676, 678, 680, 682, 684, 686, 688, 690, 692, 694, 696, 698, 700, 702, 704, 706, 708, 710, 712, 714, 716, 718, 720, 722, 724, 728, 730, 732, 734, 736, 738, 740, 742, 744, 746, 748, 750, 752, 754, 756, 758, 760, 762, 764, 766, 768, 770, 772, 774, 776, 778, 780, 782, 784, 786, 788, 790, 792, 794, 796, 798, 800, 802, 804, 806, 808, 810, 812, 814, 816, 818, 820, 822, 824, 826, 828, 830, 832, 834, 836, 838, 840, 842, 844, 846, 848, 850, 852, 854, 856, 858, 860, 862, 864, 868, 876, 878, 880, 882, 884, 886, 890, 892, 896, 900, 904, 906, 908, 910, 912, 914, 922, 932, 934, 936, 938, 940, 942, 944, 946, 948, 952, 956, 958, 960, 962, 964, 966, 970, 974, 976, 978, 980, 982, 986, 988, 990, 996, 998, 1002, 1006, 1008, 1012, 1014, 1018, 1020, 1022, 1024, 1026, 1028, 1030, 1032, 1034, 1036, 1038, 1040, 1042, 1044, 1046, 1048, 1050, 1052, 1054, 1056, 1058, 1064, 1066, 1068, 1070, 1072, 1074, 1076, 1078, 1080, 1082, 1084, 1086, 1088, 1090, 1092, 1096, 1098, 1100, 1102, 1104, 1108, 1110, 1112, 1114, 1116, 1120, 1124, 1126, 1128, 1130, 1132, 1134, 1136, 1138, 1140, 1142, 1144, 1146, 1148, 1150, 1152, 1154, 1156, 1158, 1160, 1162, 1164, 1166, 1168, 1170, 1172, 1174, 1176, 1178, 1180, 1182, 1184, 1186, 1188, 1190, 1192, 1194, 1196, 1198, 1200, 1202, 1204, 1206, 1208, 1210, 1212, 1214, 1216, 1218, 1220, 1222, 1224, 1226, 1228, 1232, 1234, 1236, 1238, 1240, 1242, 1244, 1246, 1248, 1250, 1252, 1254, 1256, 1258, 1260, 1262, 1264, 1266, 1268, 1270, 1272, 1274, 1276, 1278, 1280, 1286, 1300, 1306, 1310, 1314, 1316, 1320, 1322, 1324, 1326, 1328, 1330, 1332, 1334, 1336, 1344, 1352, 1356, 1368, 1370, 1372, 1380, 1384, 1386, 1388, 1390, 1392, 1394, 1400, 1406, 1410, 1412, 1414, 1416, 1418, 1420, 1422, 1424, 1426, 1428, 1430, 1432, 1434, 1436, 1438, 1440, 1442, 1444, 1446, 1448, 1450, 1452, 1454, 1456, 1458, 1460, 1462, 1464, 1466, 1468, 1470, 1472, 1474, 1476, 1478, 1480, 1482, 1484, 1486, 1488, 1490, 1492, 1494, 1496, 1498, 1500, 1502, 1504, 1506, 1508, 1510, 1512, 1514, 1516, 1518, 1520, 1522, 1524, 1526, 1528, 1530, 1532, 1534, 1536, 1540, 1550, 1552, 1554, 1564, 1568, 1570, 1584, 1590, 1592, 1594, 1596, 1598, 1600, 1602, 1604, 1606, 1608, 1610, 1612, 1614, 1616, 1618, 1620, 1622, 1624, 1626, 1628, 1630, 1632, 1634, 1636, 1638, 1640, 1642, 1644, 1646, 1648, 1650, 1654, 1658, 1660, 1662, 1664, 1666, 1670, 1676, 1690, 1694, 1698, 1706, 1710, 1712, 1714, 1720, 1724, 1726, 1728, 1732, 1734, 1740, 1746, 1748, 1750, 1752, 1754, 1756, 1760, 1762, 1772, 1778, 1782, 1784, 1786, 1788, 1792, 1794, 1796, 1798, 1806, 1810, 1814, 1820, 1826, 1846, 1854, 1858, 1860, 1868, 1870, 1872, 1878, 1880, 1882, 1898, 1906, 1908, 1910, 1920, 1924, 1926, 1930, 1932, 1940, 1942, 1950, 1954, 1956, 1958, 1960, 1962, 1964, 1966, 1970, 1972, 1974, 1976, 1978, 1980, 1982, 1984, 1986, 1990, 1992, 1994, 1998, 2000, 2002, 2004, 2006, 2008, 2010, 2012, 2014, 2016, 2018, 2020, 2022, 2024, 2026, 2028, 2030, 2032, 2034, 2038, 2040, 2042, 2044, 2046, 2050, 2052, 2054, 2056, 2058, 2060, 2064, 2066, 2068, 2070, 2098, 2102, 2104, 2106, 2122, 2138, 2142, 2190, 2204, 2206, 2210, 2212, 2214, 2216, 2220, 2226, 2228, 2234, 2236, 2242, 2250, 2252, 2254, 2266, 2268, 2272, 2276, 2278, 2280, 2282, 2284, 2288, 2290, 2302, 2306, 2308, 2310, 2314, 2316, 2318, 2320, 2322, 2324, 2326, 2330, 2334, 2336, 2338, 2340, 2370, 2372, 2374, 2380, 2382, 2386, 2390, 2392, 2394, 2396, 2398, 2402, 2406, 2408, 2412, 2420, 2422, 2432, 2436, 2440, 2446, 2448, 2450, 2454, 2456, 2460, 2464, 2466, 2468, 2472, 2476, 2478, 2484, 2486, 2488, 2494, 2496, 2498, 2500, 2504, 2506, 2510, 2518, 2520, 2526, 2530, 2534, 2542, 2544, 2554, 2556, 2560, 2562, 2564, 2570, 2574, 2576, 2578, 2586, 2588, 2590, 2592, 2594, 2598, 2600, 2602, 2604, 2610, 2614, 2618, 2622, 2628, 2632, 2638, 2644, 2646, 2648, 2650, 2652, 2654, 2656, 2662, 2664, 2672, 2674, 2676, 2682, 2684, 2688, 2690, 2694, 2702, 2704, 2708, 2710, 2712, 2714, 2728, 2734, 2736, 2738, 2742, 2748, 2750, 2754, 2760, 2762, 2764, 2766, 2768, 2770, 2774, 2776, 2778, 2780, 2782, 2784, 2790, 2794, 2798, 2800, 2802, 2804, 2806, 2808, 2810, 2818, 2820, 2824, 2826, 2828, 2830, 2832, 2834, 2836, 2840, 2842, 2844, 2848, 2850, 2852, 2854, 2856, 2858, 2860, 2862, 2864, 2866, 2868, or 2870.

In some embodiments, for any of the recombinant factor VIII polypeptide disclosed herein, the recombinant factor VIII polypeptide is purified or is a purified preparation.

In some embodiments, the recombinant factor VIII polypeptide comprises a full length recombinant factor VIII polypeptide of any of the recombinant factor VIII polypeptide described herein. In some embodiments, the recombinant factor VIII polypeptide comprises a “pre-pro-protein” or “pre-pro-polypeptide” of any of the recombinant factor VIII polypeptide described herein. In some embodiments, the signal sequence of the full length recombinant factor VIII polypeptide or “pre-pro-protein” or “pre-pro-polypeptide” thereof can comprise any compatible signal sequence, including synthetic, mouse (e.g., IgG), or human signal sequence. In some embodiments, the signal sequence present in the exemplary recombinant factor VIII polypeptide is replaced with a compatible signal sequence, e.g., synthetic or mouse. In some embodiments, the signal sequence comprises the signal sequence in the naturally occurring human factor VIII polypeptide.

In some embodiments, the recombinant factor VIII polypeptide is processed or cleaved to a pro-polypeptide or pro-protein form, for example, lacking the signal sequence. In some embodiments, the recombinant factor VIII polypeptide is processed or cleaved to a mature form of the recombinant factor VIII polypeptide, such as described herein. In some embodiments, the mature form of the recombinant factor VIII polypeptide comprises the active form of the recombinant factor VIII polypeptide, particularly the polypeptides (e.g., equivalent form for the light and heavy chain of factor VIII) that interact with FIX (see, e.g., Orlova et al., Acta Naturae, 2013, 5(2): 19-39:3983-3996; Lenting et al., Blood, 1998, 92(11); incorporated herein by reference).

Thus, for each and every recombinant factor VIII polypeptide described herein, the present disclosure provides a pre-pro-polypeptide, pro-polypeptide, or mature, biologically active form of the recombinant factor VIII polypeptide, including each recombinant factor VIII polypeptides disclosed in Tables 4.1, 4.2, 5.1, 5.2, 6.1, 6.2, 7.1, 7.2, 8.1, 8.2, 9.1, 9.2, 10.1, 10.2, 11.1, and 15.1.

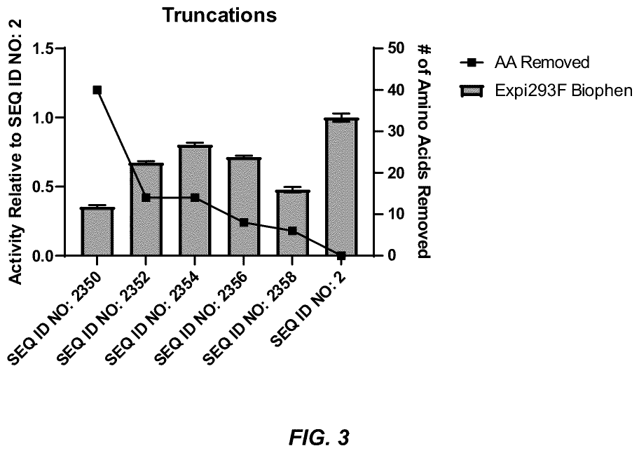

In some embodiments, the present disclosure further provides a functional or biologically active fragment of a recombinant factor VIII polypeptide described herein. In some embodiments, functional or biologically active fragments have at least 80%, 81%, 82%, 83%, 84%, 85%, 86%, 87%, 88%, 89%, 90%, 91%, 92%, 93%, 94%, 95%, 96%, 97%, 98%, or 99% of the activity of the recombinant factor polypeptide from which is was derived (i.e., the parent engineered factor VIII). In some embodiments, a functional or biologically active fragment comprises at least 80%, 81%, 82%, 83%, 84%, 85%, 86%, 87%, 88%, 89%, 90%, 91%, 92%, 93%, 94%, 95%, 96%, 97%, 98%, or 99% of the parent sequence of the recombinant factor VIII polypeptide. In some embodiments the functional fragment is truncated by less than 5, less than 10, less than 15, less than 10, less than 25, less than 30, less than 35, less than 40, less than 45, and less than 50 amino acids. Exemplary biologically active or functional fragments of the recombinant factor VIII polypeptide include amino acid sequences comprising SEQ ID NO: 2350, 2352, 2354, 2356, or 2358, whose activities are shown in FIG. 3.

In some embodiments, the functional or biologically active fragment of the recombinant factor VIII polypeptide described herein include at least a mutation or mutation set in the amino acid sequence of the recombinant factor VIII described herein. Accordingly, in some embodiments, the functional or biologically active fragments of the recombinant factor VIII displays the enhanced or improved property associated with the mutation or mutation set in the parent recombinant factor VIII polypeptide.

In some further embodiments, as discussed herein, a functional fragment or a biologically active fragment encompasses biologically processed polypeptides of a recombinant factor VIII polypeptide, such as functional or biologically active fragments produced upon expression in a mammalian cell or in a patient, or when a recombinant factor VIII polypeptide in administered to a patient. In some embodiments, a biologically processed functional or biologically active fragments of a recombinant factor VIII polypeptide resulting from cleavage or cleavages occurring between residues 760 to 773, and in some embodiments, additional cleavage or cleavages occurring between residues 391 and 392, wherein the positions are relative to the reference sequence of SEQ ID NO: 2, 252, 504, 706, 840, 1200, 1514, or 2072. In some embodiments, the functional or biologically active fragment is derived from a recombinant factor VIII polypeptide presented in Tables 4.1, 4.2, 5.1, 5.2, 6.1, 6.2, 7.1, 7.2, 8.1, 8.2, 9.1, 9.2, 10.1, 10.2, 11.1, and 15.1. In some embodiments, the functional or biologically active fragment is derived from a recombinant factor VIII polypeptide comprising an amino acid sequence of an even-numbered SEQ ID NO. selected from SEQ ID NOs: 4-2346 and 2350-2870.

### Polynucleotides Encoding Recombinant Polypeptides, Expression Vectors, and Host Cells

In another aspect, the present disclosure provides polynucleotides encoding the engineered factor VIII polypeptides described herein. In some embodiments, the polynucleotides are operatively linked to one or more heterologous or homologous regulatory sequences that control gene expression to create a recombinant polynucleotide capable of expressing the polypeptide. Expression constructs containing a heterologous polynucleotide encoding the recombinant factor VIII polypeptides can be introduced into appropriate host cells to express the corresponding recombinant factor VIII polypeptide.

As will be apparent to the skilled artisan, availability of a protein sequence and the knowledge of the codons corresponding to the various amino acids provide a description of all the polynucleotides capable of encoding the subject polypeptides. The degeneracy of the genetic code, where the same amino acids are encoded by alternative or synonymous codons, allows an extremely large number of nucleic acids to be made, all of which encode the recombinant factor VIII polypeptide. Thus, having knowledge of a particular amino acid sequence, those skilled in the art could make any number of different nucleic acids by simply modifying the sequence of one or more codons in a way which does not change the amino acid sequence of the protein. In this regard, the present invention specifically contemplates each and every possible variation of polynucleotides that could be made encoding the polypeptides described herein by selecting combinations based on the possible codon choices, and all such variations are to be considered specifically disclosed for any polypeptide described herein, including the variants provided in Tables 4.1, 4.2, 5.1, 5.2, 6.1, 6.2, 7.1, 7.2, 8.1, 8.2, 9.1, 9.2, 10.1, 10.2, 11.1, and 15.1.

In some embodiments, the codons are preferably selected to fit the host cell in which the protein is being produced. For example, preferred codons used in bacteria are used for expression in bacteria, preferred codons used in fungi are typically used for expression in fungi, and preferred codons used in mammals are used for expression in mammals and mammalian cells. In some embodiments, codon optimized polynucleotides encoding the engineered factor polypeptides contain preferred codons at about 40%, 50%, 60%, 70%, 80%, or greater than 90% of codon positions of the full length coding region. In some embodiments, the present invention provides recombinant polynucleotide sequences in which the codons are optimized for expression in human cells or tissues.

As discussed above, it is to be understood that the present disclosure provides recombinant polynucleotides encoding each and every recombinant factor VIII polypeptide, or a biologically or functionally active fragment thereof, described herein.

Accordingly, by way of example and not limitation, in some embodiments, a recombinant polynucleotide comprises a polynucleotide sequence encoding the recombinant factor VIII, or biologically active recombinant factor VIII fragments thereof, comprising an amino acid sequence having at least 70%, 75%, 80%, 81%, 82%, 83%, 84%, 85%, 86%, 87%, 88%, 89%, 90%, 91%, 92%, 93%, 94%, 95%, 96%, 97%, 98%, or 99% or more sequence identity to a reference sequence corresponding to residues 20-1457 of SEQ ID NO: 2, 252, 504, 706, 840, 1200, 1514, or 2072, or to a reference sequence corresponding to SEQ ID NO: 2, 252, 504, 706, 840, 1200, 1514, or 2072, wherein the amino acid sequence comprises one or more substitutions relative to the reference sequence corresponding to residues 20-1457 of SEQ ID NO: 2, 252, 504, 706, 840, 1200, 1514, or 2072, or relative to the reference sequence corresponding to SEQ ID NO: 2, 252, 504, 706, 840, 1200, 1514, or 2072.

In some embodiments, the recombinant polynucleotide comprises a polynucleotide sequence encoding the recombinant factor VIII polypeptide comprising an amino acid sequence having at least 70%, 75%, 80%, 81%, 82%, 83%, 84%, 85%, 86%, 87%, 88%, 89%, 90%, 91%, 92%, 93%, 94%, 95%, 96%, 97%, 98%, or 99% or more sequence identity to the reference sequence corresponding to residues 20-1457 of SEQ ID NO: 2, or to the reference sequence corresponding to SEQ ID NO: 2, wherein the amino acid sequence comprises one or more substitutions relative to the reference sequence corresponding to residues 20-1457 of SEQ ID NO: 2, or relative to the reference sequence corresponding to SEQ ID NO: 2.

In some embodiments, the recombinant polynucleotide comprises a polynucleotide sequence encoding the recombinant factor VIII polypeptide comprising an amino acid sequence having at least 70%, 75%, 80%, 81%, 82%, 83%, 84%, 85%, 86%, 87%, 88%, 89%, 90%, 91%, 92%, 93%, 94%, 95%, 96%, 97%, 98%, or 99% or more sequence identity to the reference sequence corresponding to residues 20-1457 of SEQ ID NO: 252, 504, 706, 840, 1200, 1514, or 2072, or to the reference sequence corresponding to SEQ ID NO: 252, 504, 706, 840, 1200, 1514, or 2072, wherein the amino acid sequence comprises one or more substitutions relative to the reference sequence corresponding to residues 20-1457 of SEQ ID NO: 2, or relative to the reference sequence corresponding to SEQ ID NO: 2.

In some embodiments, the recombinant polynucleotide comprises a polynucleotide sequence encoding a recombinant factor VIII polypeptide comprising an amino acid sequence having at least 70%, 75%, 80%, 81%, 82%, 83%, 84%, 85%, 86%, 87%, 88%, 89%, 90%, 91%, 92%, 93%, 94%, 95%, 96%, 97%, 98%, or 99% or more sequence identity to the reference sequence corresponding to residues 20-1457 of an even-numbered SEQ ID NO. of SEQ ID NOs. 4-2346 and 2360-2870, or to the reference sequence corresponding to an even-numbered SEQ ID NO. of SEQ ID NOs. 4-2346 and 2350-2870, wherein the amino acid sequence comprises one or more substitutions relative to the reference sequence corresponding to residues 20-1457 of SEQ ID NO: 2 or relative to the reference sequence corresponding to SEQ ID NO: 2.

In some embodiments, the recombinant polynucleotide comprises a polynucleotide sequence encoding the recombinant factor VIII polypeptide comprising an amino acid sequence comprising at least a substitution at position 13, 14, 15, 16, 17, 18, 19, 20, 21, 24, 37, 56, 57, 58, 59, 61, 63, 64, 65, 85, 96, 104, 110, 119, 127, 132, 158, 160, 164, 167, 168, 171, 175, 178, 186, 187, 205, 210, 211, 212, 215, 225, 246, 257, 260, 265, 281, 292, 294, 301, 303, 304, 306, 308, 310, 311, 312, 318, 320, 321, 325, 328, 332, 334, 335, 344, 346, 347, 349, 351, 353, 354, 355, 356, 358, 359, 362, 370, 387, 388, 390, 392, 393, 397, 407, 428, 429, 433, 435, 436, 481, 487, 488, 489, 491, 498, 499, 503, 514, 527, 533, 534, 535, 536, 538, 543, 550, 551, 555, 558, 569, 580, 581, 584, 610, 611, 612, 613, 614, 615, 616, 617, 618, 619, 620, 621, 622, 643, 646, 650, 665, 667, 676, 680, 681, 682, 683, 684, 686, 693, 696, 698, 700, 706, 707, 708, 712, 716, 724, 726, 729, 731, 732, 734, 735, 736, 737, 738, 739, 740, 744, 746, 747, 748, 749, 750, 751, 752, 753, 754, 755, 756, 757, 759, 770, 773, 775, 785, 786, 787, 790, 791, 792, 793, 794, 795, 797, 798, 799, 800, 801, 802, 805, 806, 807, 808, 809, 810, 811, 812, 815, 816, 817, 818, 819, 821, 822, 824, 825, 826, 828, 830, 831, 836, 837, 838, 839, 840, 841, 844, 866, 873, 879, 892, 896, 897, 903, 907, 914, 916, 917, 918, 919, 921, 922, 923, 924, 926, 929, 938, 939, 945, 948, 951, 968, 999, 1037, 1040, 1041, 1042, 1043, 1049, 1050, 1051, 1053, 1054, 1076, 1078, 1080, 1084, 1088, 1089, 1092, 1093, 1094, 1095, 1098, 1099, 1100, 1101, 1104, 1108, 1109, 1113, 1114, 1116, 1127, 1129, 1138, 1145, 1148, 1151, 1156, 1158, 1160, 1186, 1192, 1193, 1194, 1205, 1210, 1218, 1220, 1221, 1222, 1224, 1229, 1245, 1248, 1250, 1259, 1264, 1269, 1270, 1274, 1281, 1283, 1292, 1297, 1339, 1352, 1373, 1377, 1378, 1380, 1389, 1414, 1422, 1427, 1428, 1439, 1440, 1442, or 1452, or combinations thereof, wherein the amino acid positions are relative to the reference sequence of SEQ ID NO: 2.

In some embodiments, the recombinant polynucleotide comprises a polynucleotide sequence encoding the recombinant factor VIII polypeptide comprising an amino acid sequence comprising at least a substitution or amino acid residue, 13G/Y, 14G/M/V, 15G/S, 16A/D/G/P/V, 17S/V, 18L/R, 19G, 20L, 21M, 24V, 37H, 56E/P, 57A/L/S/T/V, 58K, 59D/G/L/M/Q/R/S/V/W, 61Q/S, 63C/E/F/G/H/I/K/L/M/N/Q/R/S/T/W/Y, 64D/G, 65A, 85Q, 96I/L/R/V/W, 104S, 110H, 119G/V, 127V, 132P/R, 158G, 160E/T, 164E, 167G/H/R, 168G/R, 171A/D/E/F/H/K/N/M/P/Q/R/S/T/V/W/Y, 175A/G/H, 178C/M/Q/V/W, 186A, 187C/G, 205R, 210P, 211G/K/P/Q/R, 212A/F/G/K/L/M/N/P/R/S/V/Y, 215E/P/S, 225R, 246D, 257I, 260E/K/T, 265R/S/W, 281A/S, 292D, 294G, 301K/M, 303G/T, 304A/E/G, 306A/D/G/P/S/T, 308P, 310G/V, 311E/G, 312L, 318C/G/K/M/Q/R/T/V, 320G/I/L/P/Q/R/S/T/V, 321Y, 325Y, 328G/Q/S, 332A/F/G/H/N/P/Q/R/T/V/W/Y, 334G, 335E/P, 344A/E/L, 346R/V, 347N, 349G, 351S, 353S, 354R, 355C, 356L/N, 358K/S, 359E/G, 362E/G/N/P/T/V/W, 370A, 387A/G/I/L/Q/R/S/T/V/W, 388A/P, 390A/E/G/L/M/P/S/T/V, 392A, 393A/F/G/H/M/Q/R/W/Y, 397G/N, 407V, 428F, 429L/S, 433A/G, 435Q, 436D/G, 481V, 487N, 488G, 489E/F/Q/R, 491E, 498A, 499C/L, 503S, 514L, 527A, 533R/V/Y, 534F/I/L/M, 535C/G/N, 536A/G/K/L/M/P/R/W, 538V, 543G/L/P/W, 550G, 551W, 555E/G/R/T, 558A/E/G/L/P/Q/R/S/T/V/W, 569T, 580S/Y, 581K/V, 584A, 610H/K/L/R/S/T, 611D/E, 612G/S/T, 613S, 614A/F/H/I/N/T, 615D/N, 616D/E/G/Q/W, 617D/E/G/N, 618C/E/G/P/Y, 619E, 620N/P, 621K/M/R/T/W, 622D, 643N, 646I, 650M, 665G, 667I, 676Q, 680L/R, 681A/C/E/F/G/L/N/R/Q/S/T/W, 682R, 683W, 684G/K, 686W, 693T, 696E, 698D/S/W/Y, 700A/E/Q/T/V, 706A/G/M/Y, 707H, 708A/K/T/V, 712G/L/M/Q/S/T/Y, 716K/L/W, 724I, 726A/L, 729T, 731I/L/R, 732C/D/E/G/L/R/W/K/M/Q/S/T/V/Y, 734I/R/S, 735D/S/V, 736F/G, 737F/G/K/L/M/N/S/V, 738A/D/E/F/H/K/N/P/R/S/T, 739G/P/R, 740G, 744A/R, 746P, 747D, 748F/G/K/N/S/V, 749A/C/D/F/G/I/K/L/M/P/Q/R/S/T/V/Y, 750A/D/G/K/M/N/P/Q/R/S/T/V/W, 751A/D/E/Y, 752D/E/F/G/L/M/Q/R/S/V, 753D, 754W, 755G/L/P/Q/S/Y, 756G/P/R/T, 757A/G/K/T, 759S, 770W, 773H, 775E/G/K/N/P/S/V, 785V, 786C/D/L, 787W, 790A/G/I/M/P/W, 791M/T, 792H/W/Y, 793G, 794R, 795C/N/S/T, 797C/G/P/Q/T/W/Y, 798C/R/T, 799A/E/G/M/P/Q/R/S/V, 800C/G/I/L/M/N/P/Q/R/T/V/W/Y, 801A/G/I/R, 802S/V, 805C/G/H/K/L/M/P/Q/R/S/V/W, 806E/G/R/S, 807C/L/P/R, 808K/S, 809A/C/G/I/K/L/N/Q/R/S/T/V/W, 810V, 811F, 812G/I/V, 815I, 816V/W, 817A/D, 818A/E/G/P/S/V/W, 819V, 821Q, 822S/T, 824W, 825L, 826G, 828K, 830M/V, 831W, 836G/R/V/W, 837G, 838G/T, 839A/G/K/P, 840G, 841W, 844H, 866A, 873L/Q/T, 879Q, 892T, 896L/V, 897G/K/R, 903E, 907D, 914V, 916C/E/I/K/L/M/Q/T, 917G, 918M/S, 919G, 921R/W, 922W, 923M, 924I/M/P/Q/W, 926T/V, 929A/Q/V, 938R/V, 939E/S, 945K, 948S, 951V, 968A/P, 999R, 1037H/M/N/P, 1040E, 1041G/H/N, 1042A/E/T, 1043E/R, 1049G/W, 1050E/P/Q/S/T, 1051E/H/K/R/S/W, 1053A/R, 1054C, 1076G/S, 1078S, 1080G/V, 1084L/N, 1088G/S, 1089F/S, 1092G, 1093A/E/G/M/N/P/Q/R/S/T/V/W/Y, 1094S, 1095G, 1098G, 1099G, 1100I/S, 1101G, 1104C/E, 1108M/S/V/W, 1109G, 1113F, 1114A/M, 1116A/G/I/L/R, 1127L/V, 1129P, 1138Q, 1145R, 1148A/C/E/L/M/N/Q/R/S, 1151S, 1156D/G/N/R, 1158N, 1160R, 1186E, 1192D, 1193A/E/G/I/L, 1194P, 1205L, 1210M/T/V/W, 1218A/G/L/M/P, 1220D/E/L/T, 1221G/S/T/V, 1222F, 1224P, 1229D, 1245R, 1248A/C/D/E/F/G/H/I/K/M/P/Q/R/S/T/W/Y, 1250A/P/S/T, 1259T, 1264L/R/W, 1269F, 1270V, 1274N/V, 1281H/K/P/Q/R/S, 1283E/H/L/S/T, 1292Y, 1297A, 1339C, 1352E, 1373S, 1377A/C/D/E/G/H/I/M/P/R/S/V/T/Y, 1378A/C/D/E, 1380A/G/W, 1389W, 1414A/C/D/E/G/H/N/P/T/W, 1422I, 1427Y, 1428G, 1439A/E/H/L/S/T, 1440A/C/E/G/S/T, 1442T, or 1452L, or combinations thereof, wherein the amino acid positions are relative to the reference sequence corresponding to SEQ ID NO: 2.

In some embodiments, the recombinant polynucleotide comprises a polynucleotide sequence encoding the recombinant factor VIII polypeptide comprising an amino acid sequence comprising at least a substitution at amino acid position 63, 171, 212, 260, 306, 317, 320, 332, 387, 390, 393, 534, 558, 681, 732, 738, 749, 750, 775, 1148, 1248, 1377, or 1439, or combinations thereof, wherein the amino acid positions are relative to the reference sequence corresponding to SEQ ID NO: 2.

In some embodiments, the recombinant polynucleotide comprises a polynucleotide sequence encoding the recombinant factor VIII polypeptide comprising an amino acid sequence comprising at least a substitution or an amino acid residue 63C/E/F/G/H/I/K/L/M/N/Q/R/S/T/W/Y, 171A/D/E/F/H/K/N/M/P/Q/R/S/T/V/W/Y, 212A/F/G/L/K/M/N/P/R/S/V/Y, 260E/K/T, 306A/D/G/P/S/T, 318C/G/K/L/M/Q/R/T/V, 320G/I/L/P/Q/R/S/T/V, 332A/F/G/H/N/P/Q/R/T/V/W/Y, 387A/G/I/L/Q/R/S/T/V/W, 390A/E/G/L/M/P/S/T/V, 393A/F/G/H/M/Q/R/W/Y, 499C/L, 534I/F/L/M, 558A/E/G/L/P/Q/R/S/T/V/W, 681A/C/E/F/G/L/N/R/Q/S/T/W, 700A/E/Q/T/V, 732C/D/E/G/L/R/W/K/M/Q/S/T/V/Y, 738A/D/E/F/H/K/N/P/R/S/T/Y, 749A/C/D/F/G/I/K/L/M/P/Q/R/S/T/V/Y, 750A/D/G/K/M/N/P/Q/R/S/T/V/W, 752D/E/F/G/L/M/Q/R/S/V, 775E/G/K/N/P/S/V, 800C/G/I/L/M/N/P/Q/R/T/V/W/Y, 805C/G/H/K/L/M/P/Q/R/S/V/W, 809A/C/G/I/K/L/N/Q/R/S/T/V/W, 1093A/E/G/M/N/P/Q/R/S/T/V/W/Y, 1148A/C/E/L/M/N/Q/R/S, 1248A/C/D/E/F/G/H/I/K/M/P/Q/R/S/T/W/Y, 1377A/C/D/E/G/H/I/M/P/R/S/V/T/Y, 1414A/C/D/E/G/H/N/P/T/W, or 1439A/E/H/L/S/T, or combinations thereof, wherein the amino acid positions are relative to the reference sequence corresponding to SEQ ID NO: 2.

In some embodiments, the recombinant polynucleotide comprises a polynucleotide sequence encoding the recombinant factor VIII polypeptide comprising an amino acid sequence comprising at least a substitution or substitution set at amino acid position(s) 1148, 85/708/1148, 798, 706, 171, 335, 63, 344, 428, 879, 59, 429, 205, 1264, 797, 1248, 873, 792, 708, 57, 96, 362, 1292, 346, 433, 65, 795, 684/798, 353, 332, 1259, 356, 436, 1245, 1145, 716, 56, 773, 734, 358, 729, 665, 359, 61, 1250, 726, 37/96/171/429/708/799/1148, 171/429/732/1148, 96/171, 171/919/1151, 171/792/797/799/1148, 171/569/1148, 96/171/429/792/797/799, 171/429/708/1422, 171/429, 321/362/732/797/799, 171/1100, 708/1148/1186, 429/724/732/1148, 171/362/795/797/799, 171/429/732, 96/171/643/797/799, 171/792/797/799, 503, 171/732, 1148/1193, 96/171/362/429/732/795/797, 334, 732, 351, 1377, 999, 306, 349, 257, 646, 435, or 58, wherein the positions are relative to the reference sequence corresponding to SEQ ID NO: 2.

In some embodiments, the recombinant polynucleotide comprises a polynucleotide sequence encoding the recombinant factor VIII polypeptide comprising an amino acid sequence comprising a substitution at an amino acid position set forth in Tables 4.1, 4.2, 5.1, 5.2, 6.1, 6.2, 7.1, 7.2, 8.1, 8.2, 9.1, 9.2, 10.1, 10.2, 11.1, and 15.1, wherein the amino acid positions are relative to the reference sequence corresponding to SEQ ID NO: 2.

In some embodiments, the recombinant polynucleotide comprises a polynucleotide sequence encoding the recombinant factor VIII polypeptide comprising an amino acid sequence comprising at least one substitution set forth in Tables 4.1, 4.2, 5.1, 5.2, 6.1, 6.2, 7.1, 7.2, 8.1, 8.2, 9.1, 9.2, 10.1, 10.2, 11.1, and 15.1, wherein the amino acid positions are relative to the reference sequence corresponding to SEQ ID NO: 2.

In some embodiments, the recombinant polynucleotide comprises a polynucleotide sequence encoding the recombinant factor VIII polypeptide comprising an amino acid sequence comprising at least a substitution or substitution set at the position(s) set forth in Tables 4.1, 4.2, 5.1, 5.2, 6.1, 6.2, 7.1, 7.2, 8.1, 8.2, 9.1, 9.2, 10.1, 10.2, 11.1, and 15.1, wherein the amino acid positions are relative to the reference sequence corresponding to SEQ ID NO: 2.

In some embodiments, the recombinant polynucleotide comprises a polynucleotide sequence encoding the recombinant factor VIII polypeptide comprising an amino acid sequence comprising at least a substitution or substitution set of a variant set forth in Tables 4.1, 4.2, 5.1, 5.2, 6.1, 6.2, 7.1, 7.2, 8.1, 8.2, 9.1, 9.2, 10.1, 10.2, 11.1, and 15.1, wherein the amino acid positions are relative to the reference sequence corresponding to SEQ ID NO: 2.

In some embodiments, the recombinant polynucleotide comprises a polynucleotide sequence encoding the recombinant factor VIII polypeptide comprising an amino acid sequence having at least 70%, 75%, 80%, 81%, 82%, 83%, 84%, 85%, 86%, 87%, 88%, 89%, 90%, 91%, 92%, 93%, 94%, 95%, 96%, 97%, 98%, or 99% or more sequence identity to the reference sequence corresponding to residues 20-1457 of SEQ ID NO: 252, 504, 706, 840, 1200, 1514, or 2072, or to the reference sequence corresponding to SEQ ID NO: 252, 504, 706, 840, 1200, 1514, or 2072.

In some embodiments, the recombinant polynucleotide comprises a polynucleotide sequence encoding the recombinant factor VIII polypeptide comprising an amino acid sequence having at least 70%, 75%, 80%, 81%, 82%, 83%, 84%, 85%, 86%, 87%, 88%, 89%, 90%, 91%, 92%, 93%, 94%, 95%, 96%, 97%, 98%, or 99% or more sequence identity to the reference sequence corresponding to residues 20-1457 of an even-numbered SEQ ID NO. of SEQ ID NOs. 4-2346 and 2360-2870, or to the reference sequence corresponding to an even-numbered SEQ ID NO. of SEQ ID NOs. 4-2346 and 2350-2870.

In some embodiments, the recombinant polynucleotide comprises a polynucleotide sequence encoding the recombinant factor VIII polypeptide comprising an amino acid sequence having at least 75%, 80%, 81%, 82%, 83%, 84%, 85%, 86%, 87%, 88%, 89%, 90%, 91%, 92%, 93%, 94%, 95%, 96%, 97%, 98%, or 99% or more sequence identity to the reference sequence corresponding to residues 20-1457 of SEQ ID NO: 252, 504, 706, 840, 1200, 1514, or 2072, or to the reference sequence corresponding to SEQ ID NO: 252, 504, 706, 840, 1200, 1514, or 2072, wherein the amino acid sequence comprises one or more substitutions relative to the reference sequence corresponding to residues 20-1457 of SEQ ID NO: 252, 504, 706, 840, 1200, 1514, or 2072, or relative to the reference sequence corresponding to SEQ ID NO: 252, 504, 706, 840, 1200, 1514, or 2072.

In some embodiments, the recombinant polynucleotide comprises a polynucleotide sequence encoding the recombinant factor VIII polypeptide comprising an amino acid sequence comprising at least a substitution at amino acid position 13, 14, 15, 16, 17, 18, 19, 20, 21, 24, 37, 56, 57, 58, 59, 61, 63, 64, 65, 85, 96, 104, 110, 119, 127, 132, 158, 160, 164, 167, 168, 171, 175, 178, 186, 187, 205, 210, 211, 212, 215, 225, 246, 257, 260, 265, 281, 292, 294, 301, 303, 304, 306, 308, 310, 311, 312, 318, 320, 321, 325, 328, 332, 334, 335, 344, 346, 347, 349, 351, 353, 354, 355, 356, 358, 359, 362, 370, 387, 388, 390, 392, 393, 397, 407, 428, 429, 433, 435, 436, 481, 487, 488, 489, 491, 498, 499, 503, 514, 527, 533, 534, 535, 536, 538, 543, 550, 551, 555, 558, 569, 580, 581, 584, 610, 611, 612, 613, 614, 615, 616, 617, 618, 619, 620, 621, 622, 643, 646, 650, 665, 667, 676, 680, 681, 682, 683, 684, 686, 693, 696, 698, 700, 706, 707, 708, 712, 716, 724, 726, 729, 731, 732, 734, 735, 736, 737, 738, 739, 740, 744, 746, 747, 748, 749, 750, 751, 752, 753, 754, 755, 756, 757, 759, 770, 773, 775, 785, 786, 787, 790, 791, 792, 793, 794, 795, 797, 798, 799, 800, 801, 802, 805, 806, 807, 808, 809, 810, 811, 812, 815, 816, 817, 818, 819, 821, 822, 824, 825, 826, 828, 830, 831, 836, 837, 838, 839, 840, 841, 844, 866, 873, 879, 892, 896, 897, 903, 907, 914, 916, 917, 918, 919, 921, 922, 923, 924, 926, 929, 938, 939, 945, 948, 951, 968, 999, 1037, 1040, 1041, 1042, 1043, 1049, 1050, 1051, 1053, 1054, 1076, 1078, 1080, 1084, 1088, 1089, 1092, 1093, 1094, 1095, 1098, 1099, 1100, 1101, 1104, 1108, 1109, 1113, 1114, 1116, 1127, 1129, 1138, 1145, 1148, 1151, 1156, 1158, 1160, 1186, 1192, 1193, 1194, 1205, 1210, 1218, 1220, 1221, 1222, 1224, 1229, 1245, 1248, 1250, 1259, 1264, 1269, 1270, 1274, 1281, 1283, 1292, 1297, 1339, 1352, 1373, 1377, 1378, 1380, 1389, 1414, 1422, 1427, 1428, 1439, 1440, 1442, or 1452, or combinations thereof, wherein the amino acid positions are relative to the reference sequence corresponding to SEQ ID NO: 252, 504, 706, 840, 1200, 1514, or 2072.

In some embodiments, the recombinant polynucleotide comprises a polynucleotide sequence encoding the recombinant factor VIII polypeptide comprising an amino acid sequence comprising at least a substitution or an amino acid residue 13G/Y, 14G/M/V, 15G/S, 16A/D/G/P/V, 17S/V, 18L/R, 19G, 20L, 21M, 24V, 37H, 56E/P, 57A/L/S/T/V, 58K, 59D/G/L/M/Q/R/S/V/W, 61Q/S, 63C/E/F/G/H/I/K/L/M/N/Q/R/S/T/W/Y, 64D/G, 65A, 85Q, 96I/L/R/V/W, 104S, 110H, 119G/V, 127V, 132P/R, 158G, 160E/T, 164E, 167G/H/R, 168G/R, 171A/D/E/F/H/K/N/M/P/Q/R/S/T/V/W/Y, 175A/G/H, 178C/M/Q/V/W, 186A, 187C/G, 205R, 210P, 211G/K/P/Q/R, 212A/F/G/L/K/M/N/P/R/S/V/Y, 215E/P/S, 225R, 246D, 257I, 260E/K/T, 265R/S/W, 281A/S, 292D, 294G, 301K/M, 303G/T, 304A/E/G, 306A/D/G/P/S/T, 308P, 310G/V, 311E/G, 312L, 318C/G/K/L/M/Q/R/T/V, 320G/I/L/P/Q/R/S/T/V, 321Y, 325Y, 328G/Q/S, 332A/F/G/H/N/P/Q/R/T/V/W/Y, 334G, 335E/P, 344A/E/L, 346R/V, 347N, 349G, 351S, 353S, 354R, 355C, 356L/N, 358K/S, 359E/G, 362E/G/N/P/T/V/W, 370A, 387A/G/I/L/Q/R/S/T/V/W, 388A/P, 390A/E/G/L/M/P/S/T/V, 392A, 393A/F/G/H/M/Q/R/W/Y, 397G/N, 407V, 428F, 429L/S, 433A/G, 435Q, 436D/G, 481V, 487N, 488G, 489E/F/Q/R, 491E, 498A, 499C/L, 503S, 514L, 527A, 533R/V/Y, 534I/F/L/M, 535C/G/N, 536A/G/K/L/M/P/R/W, 538V, 543G/L/P/W, 550G, 551W, 555E/G/R/T, 558A/E/G/L/P/Q/R/S/T/V/W, 569T, 580S/Y, 581K/V, 584A, 610H/K/L/R/S/T, 611D/E, 612G/S/T, 613S, 614A/F/H/I/N/T, 615D/N, 616D/E/G/Q/W, 617D/E/G/N, 618C/E/G/P/Y, 619E, 620N/P, 621K/M/R/T/W, 622D, 643N, 646I, 650M, 665G, 667I, 676Q, 680L/R, 681A/C/E/F/G/L/N/R/Q/S/T/W, 682R, 683W, 684G/K, 686W, 693T, 696E, 698D/S/W/Y, 700A/E/Q/T/V, 706A/G/M/Y, 707H, 708A/K/T/V, 712G/L/M/Q/S/T/Y, 716K/L/W, 724I, 726A/L, 729T, 731I/L/R, 732C/D/E/G/L/R/W/K/M/Q/S/T/V/Y, 734I/R/S, 735D/S/V, 736F/G, 737F/G/K/L/M/N/S/V, 738A/D/E/F/H/K/N/P/R/S/T/Y, 739G/P/R, 740G, 744A/R, 746P, 747D, 748F/G/K/N/S/V, 749A/C/D/F/G/I/K/L/M/P/Q/R/S/T/V/Y, 750A/D/G/K/M/N/P/Q/R/S/T/V/W, 751A/D/E/Y, 752D/E/F/G/L/M/Q/R/S/V, 753D, 754W, 755G/L/P/Q/S/Y, 756G/P/R/T, 757A/G/K/T, 759S, 770W, 773H, 775E/G/K/N/P/S/V, 785V, 786C/D/L, 787W, 790A/G/I/M/P/W, 791M/T, 792H/W/Y, 793G, 794R, 795C/N/S/T, 797C/G/P/Q/T/W/Y, 798C/R/T, 799A/E/G/M/P/Q/R/S/V, 800C/G/I/L/M/N/P/Q/R/T/V/W/Y, 801A/G/I/R, 802S/V, 805C/G/H/K/L/M/P/Q/R/S/V/W, 806E/G/R/S, 807C/L/P/R, 808K/S, 809A/C/G/I/K/L/N/Q/R/S/T/V/W, 810V, 811F, 812G/I/V, 815I, 816V/W, 817A/D, 818A/E/G/P/S/V/W, 819V, 821Q, 822S/T, 824W, 825L, 826G, 828K, 830M/V, 831W, 836G/R/V/W, 837G, 838G/T, 839A/G/K/P, 840G, 841W, 844H, 866A, 873L/Q/T, 879Q, 892T, 896L/V, 897G/K/R, 903E, 907D, 914V, 916C/E/I/K/L/M/Q/T, 917G, 918M/S, 919G, 921R/W, 922W, 923M, 924I/M/P/Q/W, 926T/V, 929A/Q/V, 938R/V, 939E/S, 945K, 948S, 951V, 968A/P, 999R, 1037H/M/N/P, 1040E, 1041G/H/N, 1042A/E/T, 1043E/R, 1049G/W, 1050E/P/Q/S/T, 1051E/H/K/R/S/W, 1053A/R, 1054C, 1076G/S, 1078S, 1080G/V, 1084L/N, 1088G/S, 1089F/S, 1092G, 1093A/E/G/M/N/P/Q/R/S/T/V/W/Y, 1094S, 1095G, 1098G, 1099G, 1100I/S, 1101G, 1104C/E, 1108M/S/V/W, 1109G, 1113F, 1114A/M, 1116A/G/I/L/R, 1127L/V, 1129P, 1138Q, 1145R, 1148A/C/E/L/M/N/Q/R/S, 1151S, 1156D/G/N/R, 1158N, 1160R, 1186E, 1192D, 1193A/E/G/I/L, 1194P, 1205L, 1210M/T/V/W, 1218A/G/L/M/P, 1220D/E/L/T, 1221G/S/T/V, 1222F, 1224P, 1229D, 1245R, 1248A/C/D/E/F/G/H/I/K/M/P/Q/R/S/T/W/Y, 1250A/P/S/T, 1259T, 1264L/R/W, 1269F, 1270V, 1274N/V, 1281H/K/P/Q/R/S, 1283E/H/L/S/T, 1292Y, 1297A, 1339C, 1352E, 1373S, 1377A/C/D/E/G/H/I/M/P/R/S/V/T/Y, 1378A/C/D/E, 1380A/G/W, 1389W, 1414A/C/D/E/G/H/N/P/T/W, 1422I, 1427Y, 1428G, 1439A/E/H/L/S/T, 1440A/C/E/G/S/T, 1442T, or 1452L, or combinations thereof, wherein the amino acid positions are relative to the reference sequence corresponding to SEQ ID NO: 252, 504, 706, 840, 1200, 1514, or 2072.

In some embodiments, the recombinant polynucleotide comprises a polynucleotide sequence encoding the recombinant factor VIII polypeptide comprising an amino acid sequence comprising at least a substitution or substitution set at amino acid position 63, 171, 212, 260, 306, 317, 320, 332, 387, 390, 393, 534, 558, 681, 732, 738, 749, 750, 775, 1148, 1248, 1377, or 1439, or combinations thereof, wherein the amino acid positions are relative to the reference sequence corresponding to SEQ ID NO: 252, 504, 706, 840, 1200, 1514, or 2072.

In some embodiments, the recombinant polynucleotide comprises a polynucleotide sequence encoding the recombinant factor VIII polypeptide comprising an amino acid sequence having at least 70%, 75%, 80%, 81%, 82%, 83%, 84%, 85%, 86%, 87%, 88%, 89%, 90%, 91%, 92%, 93%, 94%, 95%, 96%, 97%, 98%, or 99% or more sequence identity to the reference sequence corresponding to residues 20-1457 of SEQ ID NO: 252, or to the reference sequence corresponding to SEQ ID NO: 252, wherein the amino acid sequence comprises one or more substitutions relative to the reference sequence corresponding to residues 20-1457 of SEQ ID NO: 252, or relative to the reference sequence corresponding to SEQ ID NO: 252.

In some embodiments, the recombinant polynucleotide comprises a polynucleotide sequence encoding the recombinant factor VIII polypeptide comprising an amino acid sequence comprising at least a substitution or substitution set at amino acid position(s) 1380, 388, 1043, 610, 1274, 1220, 1442, 1193, 621, 1054, 755, 1041, 748, 1049, 1221, 1051, 612, 1037, 616, 387, 489, 750, 1224, 618, 1050, 1053, 896, 897, 749, 260, 481, 613, 756, 63, 265, 752, 1218, 1378, 614, 1440, 1439, 1205, 1222, 1389, 1269, 390, 397/1218, 757, 1373, 346, 63/1248, 349, 306/355/1422, 1422, 306/346/708, 1148, 171/346/1248, 63/797/799/1248/1422, 171/799, 362, 1248, 171/306/346/999/1248, 171/1148, 734/799/1248/1422, 63/732, 63/171/306/346/732, 171, 171/1422, 63/362/732/799/1248, 999/1148/1422, 346/349, 306/349, 63/171/1148/1151, 306, 344/349/354/1250, 1250, 63/306/344/349/354/503/1248, 59/63/332/349/503/706/708, 63/306/344/349/354/706/1248, 59/63, 332/706/708/1248/1250, 503, 59/63/349/354, 59/349/708, 59/306/344/1248, 344/1248/1250, 708, 63/344/349, 59, 63/344/349/354, 706, 59/306/770, 63/347/354/1248, or 354/503, wherein the amino acid positions are relative to the reference sequence corresponding to SEQ ID NO: 252.

In some embodiments, the recombinant polynucleotide comprises a polynucleotide sequence encoding the recombinant factor VIII polypeptide comprising an amino acid sequence having at least 70%, 75%, 80%, 81%, 82%, 83%, 84%, 85%, 86%, 87%, 88%, 89%, 90%, 91%, 92%, 93%, 94%, 95%, 96%, 97%, 98%, or 99% or more sequence identity to the reference sequence corresponding to residues 20-1457 of SEQ ID NO: 504, or to the reference sequence corresponding to SEQ ID NO: 504, wherein the amino acid sequence comprises one or more substitutions relative to the reference sequence corresponding to residues 20-1457 of SEQ ID NO: 504, or relative to the reference sequence corresponding to SEQ ID NO: 504.

In some embodiments, the recombinant polynucleotide comprises a polynucleotide sequence encoding the recombinant factor VIII polypeptide comprising an amino acid sequence comprising at least a substitution or substitution set at amino acid position(s) 750, 1051, 260, 756, 1193, 332, 897, 387, 1037, 306/749, 1270, 489, 487, 488, 903, 171/1248/1440, 260/1148/1378, 171/260/387/750/1248, 171/260, 171/260/387/750/1051/1148, 171, 750/1051/1148, 171/260/756, 171/1248, 171/1148, 59, 1440, 332/750/1248, 756/1248, 1248, 171/387/1248/1440, 750/1440, 59/171/750, 171/1193/1248, 750/1148/1193/1440, 1148, 171/387/1051, 748, 306, 306/387/390/749/1220, 390, 306/390/1037, 306/612/749, 387/390/749, 63/390/503/749, 1220, 63/306, 749, 306/390/749/1037, 306/749/1220, 306/387/390/612/749/1037, or 328, wherein the amino acid positions are relative to the reference sequence corresponding to SEQ ID NO: 504.

In some embodiments, the recombinant polynucleotide comprises a polynucleotide sequence encoding the recombinant factor VIII polypeptide comprising an amino acid sequence having at least 70%, 75%, 80%, 81%, 82%, 83%, 84%, 85%, 86%, 87%, 88%, 89%, 90%, 91%, 92%, 93%, 94%, 95%, 96%, 97%, 98%, or 99% or more sequence identity to the reference sequence corresponding to residues 20-1457 of SEQ ID NO: 706, or to the reference sequence corresponding to SEQ ID NO: 706, wherein the amino acid sequence comprises one or more substitutions relative to the reference sequence corresponding to residues 20-1457 of SEQ ID NO: 706, or relative to the reference sequence corresponding to SEQ ID NO: 706.

In some embodiments, the recombinant polynucleotide comprises a polynucleotide sequence encoding the recombinant factor VIII polypeptide comprising an amino acid sequence comprising at least a substitution or substitution set at amino acid position(s) 1051, 1221, 1218, 387, 749/1218, 332, 897, 696, 816, 824, 826, 393, 825, 698, 332/387/390/1148, 328/387/749, 749, 306/328/332/390/1051/1193/1218, 63/306/332/1051/1148, 63/306/332/387/1051, 387/390/1051, 186/306/328/332/390/749/1051/1148/1193, 387/390/756/1051, 306/390/1051, 306/332/390/749, 306/387/390/1148, 306/328/332/387/390/749/1051/1148/1193, 332/387/390/749/1148, 387/390/749, 306/387/749/1051/1148, 387/749, 306/945/1051/1193/1221, 306/390/749/1051, 63/306/332/390, 306/387/390/749/1148/1193/1221, 306/332/387/390/749/1218, 63/306/332/390/1051/1148/1193/1218, 924, 755, 752, 751, 923, 797, 754, 793, 753, 922, 896, 938, 775, 732, 810, 747, 393/407, 819, 787, 802, 811, 392, or 397, wherein the amino acid positions are relative to the reference sequence corresponding to SEQ ID NO: 706.

In some embodiments, the recombinant polynucleotide comprises a polynucleotide sequence encoding the recombinant factor VIII polypeptide comprising an amino acid sequence having at least 70%, 75%, 80%, 81%, 82%, 83%, 84%, 85%, 86%, 87%, 88%, 89%, 90%, 91%, 92%, 93%, 94%, 95%, 96%, 97%, 98%, or 99% or more sequence identity to the reference sequence corresponding to residues 20-1457 of SEQ ID NO: 840, or to the reference sequence corresponding to SEQ ID NO: 840, wherein the amino acid sequence comprises one or more substitutions relative to the reference sequence corresponding to residues 20-1457 of SEQ ID NOs: 840, or relative to the reference sequence corresponding to SEQ ID NO: 840.

In some embodiments, the recombinant polynucleotide comprises a polynucleotide sequence encoding the recombinant factor VIII polypeptide comprising an amino acid sequence comprising at least a substitution or substitution set at amino acid position(s) 620, 491, 1229, 615, 1283, 822, 328, 212, 818, 555/684, 828, 550, 830, 839, 13, 14, 17, 836, 1210, 1281, 64, 682, 318, 20, 21, 325, 533, 19, 817, 831, 225, 1156, 393/747, 753, 393/732/751, 63/797, 622, 617, 907, 1040, 611, 488, 1192, 1194, 619, 16, 802, 211, 555, 536, 527, 841, 320, 837, 558, 914, 312, 681, 838, 15, 1427, 18, 683, 24, 534, 840, 1160, 551, 393, 393/747/752/811/819/923/1148, 393/751/775, 63/393/732/775/1148, 393/732/1148, 775/1148, 393/752/753/775/793/797, 393/923, 393/923/1148, 393/747/751, 393/747/753/775/1148, 752/923/1051, 63/775, 63/393/752/775/793/1051/1053, 752/753/775/793/819, 63/1051, 63/747/752/753/775/811/819/923/1051, 393/752/787, 751, 63/1148, 63/747/752/753/819/923/1148, 1148, 747/752/753/793/811, 63, 797/1148, 751/752/753/1051, 63/732/1051, 752/797/815/923/1051/1148, or 747/753, wherein the amino acid positions are relative to the reference sequence corresponding to SEQ ID NO: 840.

In some embodiments, the recombinant polynucleotide comprises a polynucleotide sequence encoding the recombinant factor VIII polypeptide comprising an amino acid sequence having at least 70%, 75%, 80%, 81%, 82%, 83%, 84%, 85%, 86%, 87%, 88%, 89%, 90%, 91%, 92%, 93%, 94%, 95%, 96%, 97%, 98%, or 99% or more sequence identity to the reference sequence corresponding to residues 20-1457 of SEQ ID NO: 1200, or to the reference sequence corresponding to SEQ ID NO: 1200, wherein the amino acid sequence comprises one or more substitutions relative to the reference sequence corresponding to residues 20-1457 of SEQ ID NOs: 1200, or relative to the reference sequence corresponding to SEQ ID NO: 1200.

In some embodiments, the recombinant polynucleotide comprises a polynucleotide sequence encoding the recombinant factor VIII polypeptide comprising an amino acid sequence comprising at least a substitution or substitution set at amino acid position(s) 681, 622, 617, 558, 536, 211, 320, 839, 797, 533, 751, 303, 951, 164, 818, 807, 799, 735, 794, 926, 755, 921, 801, 1094, 968, 929, 924, 698, 168, 581, 916, 1428, 805, 1114, 311, 737, 693, 809, 757, 1099, 676, 740, 791, 211/536/622/681, 536/558, 211/536/555/617/732, 211/681/732, 211/536/555, 555, 211/536, 211/212, 211/536/617/681/732, 211/536/558/617/681, 555/732, 536/555/558/617/732, 536/617/681/732, 536/555, 536/622/681, 732, 212/320/533/617/681, 211/312/533/534/617/619/681/839, 212, 211/212/320/619/681/839, 797/839, 212/312/320/533/534/617/681, 558/619/681, 320/558/681, 212/320/534/619/681/797/839, 320/558/619/732/839, 211/212/312/534/558/619/681/732/839, 320/534/558/619/732/797, 558/617/619/681, 211/534/558/611/681, 211/320/617/619/681, 558/617/681/797, 320/534/619/732, 211/619/681, 211/212/534/558/619/732/839, 212/320/619/681/797/839, 212/320/611/839, 320/558/619/681, 312/533/617/681/797/839, 558/611/681, 312, 211/320/558/619/681/732/797/839, 320/558/732/797, 533/558/611/619/716/732/839, 312/681, 320/534/619/681, 211/320/533/536/619, 558/617/681/839, 212/320/534/558/681, 533/534/681/839, 212/534/619/681, 617/619/681, 211/212/320/558/617/681, 212/534/558/681/797, 212/320/534, 211/312/619, 212/312/619/839, 210/212/320/558/681, 212/312/534/681/797/839, 211/320/558/839, 294, 759, 538, or 1109, wherein the amino acid positions are relative to the reference sequence corresponding to SEQ ID NO: 1200.

In some embodiments, the recombinant polynucleotide comprises a polynucleotide sequence encoding the recombinant factor VIII polypeptide comprising an amino acid sequence having at least 70%, 75%, 80%, 81%, 82%, 83%, 84%, 85%, 86%, 87%, 88%, 89%, 90%, 91%, 92%, 93%, 94%, 95%, 96%, 97%, 98%, or 99% or more sequence identity to the reference sequence corresponding to residues 20-1457 of SEQ ID NO: 1514, or to the reference sequence corresponding to SEQ ID NO: 1514, wherein the amino acid sequence comprises one or more substitutions relative to the reference sequence corresponding to residues 20-1457 of SEQ ID NOs: 1514, or relative to the reference sequence corresponding to SEQ ID NO: 1514.

In some embodiments, the recombinant polynucleotide comprises a polynucleotide sequence encoding the recombinant factor VIII polypeptide comprising an amino acid sequence having at least a substitution or substitution set at amino acid position(s) 1042, 1041, 1051, 1283, 1281, 916, 187, 215, 178, 1050, 1210, 104, 615, 610, 536, 618, 621, 614, 110, 301, 1078, 812, 786, 127, 175, 1093, 1088, 650, 535, 806, 738, 700, 132, 1076, 1084, 498, 756, 1108, 746, 160, 543, 310, 584, 918, 1452, 790, 292, 800, 167, 892, 304, 1116, 752, 281, 917, 712, 1080, 806/1352, 1095, 744, 1098, 948, 119, 785, 1113, 667, 158, 580, 707, 731, 1100, 1089, 1092, 939, 1104, 736, 1297, 1127, 686, 1129, 1138, 786/821, 680, 1101, 808, 739, 308, 536/555/619/732/799/805, 555/619/801, 555/619/799, 619/799/801/805, 536/555/619/732/799, 799/805, 801, 246/619/799/801, 536/555/619, 619/839, 619/799/805, 536/801/805, 555/619/799/805, 619/801/805, 555/619, 536/619/801, 801/805, 536/619/799/805, 555/619/732/799/801/805, 749, 616, 611, 370/614, 1156, 1158, 555/681/737/799, 536/619/809/1109, 536/555/619/737/799/809/839/916/921, 916/1109, 536/619/681/799/916/1109, 555/619/681/807/1109, 555/619/681/737/799, 619/681/799/916/1109, 619/681, 555/921, 619/681/916, 555/619/681/799, 555, 732, 619/799, 619, 681, 555/916, 839, or 799, wherein the amino acid positions are relative to the reference sequence corresponding to SEQ ID NO: 1514.

In some embodiments, the recombinant polynucleotide comprises a polynucleotide sequence encoding the recombinant factor VIII polypeptide comprising an amino acid sequence having at least 70%, 75%, 80%, 81%, 82%, 83%, 84%, 85%, 86%, 87%, 88%, 89%, 90%, 91%, 92%, 93%, 94%, 95%, 96%, 97%, 98%, or 99% or more sequence identity to the reference sequence corresponding to residues 20-1457 of SEQ ID NO: 2072, or to the reference sequence corresponding to SEQ ID NO: 2072, wherein the amino acid sequence comprises one or more substitutions relative to the reference sequence corresponding to residues 20-1457 of SEQ ID NOs: 2072, or relative to the reference sequence corresponding to SEQ ID NO: 2072.

In some embodiments, the recombinant polynucleotide comprises a polynucleotide sequence encoding the recombinant factor VIII polypeptide comprising an amino acid sequence comprising at least a substitution or substitution set at amino acid position(s) 167, 167/738/1076/1093, 167/738/752/800/805/808/809/1116, 167/800/809/1076, 167/318, 167/318/800/805/808/809, 167/318/681, 167/318/681/700/805/808/809/1076/1116, 167/318/700, 167, 167/738/1076/1093, 167/738/752/800/805/808/809/1116, 167/800/809/1076, 167/318, 167/318/800/805/808/809, 167/318/681, 167/318/681/700/805/808/809/1076/1116, 167/318/700, 167/318/700/1076, 167/318/700/738/739/1076, 167/681/738/800/808/809, 167/178/700/752/1093, 167/700, 167/700/738/739/1093, 167/700/738/752/1093, 167/700/738/752/805/808/809/1093/1116, 808/809, 1076, 738, 738/808/809, 738/1076, 738/739, 738/739/809, 738/739/809/1093/1116, 738/739/1093, 738/739/752/1093, 738/739/799/800/1093, 738/739/1116, 738/800, 738/1093, 738/752, 738/752/1093, 738/752/799, 738/799/809/1093, 738/799/1093, 739, 739/800/809, 800, 800/805/809, 809, 809/1093, 809/1093/1116, 809/1116, 318, 318/1076, 318/1076/1116, 318/739/805/808/809/1076, 318/499/700/738/809, 318/1093, 318/752/1093, 318/1116, 318/543/739, 318/543/681/752/1116, 318/543/1093, 318/700/738/1093/1414, 681, 681/738, 681/738/739/1093, 681/738/739/752/799/800/809/1116/1339, 681/738/739/1116, 681/738/800, 681/800/805/809, 681/809/1093, 681/752/1093, 681/752/1093, 681/700, 681/700/738/739/1093/1116, 681/700/809, 681/700/1093, 681/700/752/808/809/1093/1116, 681/700/752/1093, 681/700/844, 681/700/805/809, 1093, 1093/1116, 752, 752/1076, 752/800/809, 752/800/1093, 752/809, 752/866, 752/1093, 752/1116, 178/318/739/752/809/1076/1116, 178/318/800/809, 178/318/681/800/808/809/1076, 178/681, 178/681/738/1116, 178/752/809, 178/700/800/805/808/809, 178/805/809/866, 1116, 543, 543/738/739/809, 543/738/809/1093, 543/739/1093, 543/800/1093/1116, 543/809/1093, 543/809/1093/1116, 543/681/738/739/752/809/1093, 543/681/738/739/752/1093, 543/681/739/752/800/805/809/1093, 543/681/700/738/739/800/1093/1116, 543/681/700/738/739/805/808/809, 543/681/700/752, 543/681/700/752/809/1093, 543/1093, 543/700, 543/700/738/739/752/800/1093/1116, 543/700/809, 543/700/1093, 543/700/752, 543/700/752/800/1093, 543/700/752/809/1116, 543/700/752/1093, 700, 700/738/739/800/1093, 700/738/739/799/1093, 700/738/800/1093, 700/738/1116, 700/809, 700/809/1093, 700/1093, 700/1093/1116, 700/752, 700/752/808/809/1093/1116, 700/752/809/1093, 700/752/799/1116, 700/752/805/809/1093, 700/799/800, 700/1116, 700/805/808/809/1076, 514/543/681/805/808/809, 805/808/809, or 805/809/1116, wherein the amino acid positions are relative to the reference sequence corresponding to SEQ ID NO: 2072.

In some embodiments, the recombinant polynucleotide comprises a polynucleotide sequence encoding the recombinant factor VIII polypeptide comprising an amino acid sequence comprising a substitution at an amino position set forth in Tables 4.1, 4.2, 5.1, 5.2, 6.1, 6.2, 7.1, 7.2, 8.1, 8.2, 9.1, 9.2, 10.1, 10.2, 11.1, and 15.1, wherein the amino acid positions are relative to the reference sequence corresponding to SEQ ID NO: 2, 252, 504, 706, 840, 1200, 1514, or 2072.

In some embodiments, the recombinant polynucleotide comprises a polynucleotide sequence encoding the recombinant factor VIII polypeptide comprising an amino acid sequence comprising at least one substitution set forth in Tables 4.1, 4.2, 5.1, 5.2, 6.1, 6.2, 7.1, 7.2, 8.1, 8.2, 9.1, 9.2, 10.1, 10.2, 11.1, and 15.1, wherein the amino acid positions are relative to the reference sequence corresponding to SEQ ID NO: 2, 252, 504, 706, 840, 1200, 1514, or 2072.

In some embodiments, the recombinant polynucleotide comprises a polynucleotide sequence encoding the recombinant factor VIII polypeptide comprising an amino acid sequence comprising at least a substitution or substitution set forth in Tables 4.1, 4.2, 5.1, 5.2, 6.1, 6.2, 7.1, 7.2, 8.1, 8.2, 9.1, 9.2, 10.1, 10.2, 11.1, and 15.1, wherein the amino acid positions are relative to the reference sequence corresponding to SEQ ID NO: 2, 252, 504, 706, 840, 1200, 1514, or 2072.

In some embodiments, the recombinant polynucleotide comprises a polynucleotide sequence encoding the recombinant factor VIII polypeptide comprising an amino acid sequence comprising at least a substitution or substitution of a variant set forth in Tables 4.1, 4.2, 5.1, 5.2, 6.1, 6.2, 7.1, 7.2, 8.1, 8.2, 9.1, 9.2, 10.1, 10.2, 11.1, and 15.1, wherein the amino acid positions are relative to the reference sequence corresponding to SEQ ID NO: 2, 252, 504, 706, 840, 1200, 1514, or 2072.

In some embodiments, the recombinant polynucleotide comprises a polynucleotide sequence encoding the recombinant factor VIII polypeptide comprising an amino acid sequence comprising residues 20-1457 of the sequence of an even numbered SEQ ID NO. set forth in Tables 4.1, 4.2, 5.1, 5.2, 6.1, 6.2, 7.1, 7.2, 8.1, 8.2, 9.1, 9.2, 10.1, 10.2, 11.1, and 15.1, or comprising the sequence of an even numbered SEQ ID NO. set forth in Tables 4.1, 4.2, 5.1, 5.2, 6.1, 6.2, 7.1, 7.2, 8.1, 8.2, 9.1, 9.2, 10.1, 10.2, 11.1, and 15.1.

In some embodiments, the recombinant polynucleotide comprises a polynucleotide sequence encoding the recombinant factor VIII polypeptide comprising an amino acid sequence comprising residues 20-1457 of an even-numbered SEQ ID NO. of SEQ ID NOs: 4-2346 and 2360-2870, or comprising an even-numbered SEQ ID NO. of SEQ ID NOs: 4-2346 and 2350-2870.

In some embodiments, the recombinant polynucleotide comprises a polynucleotide sequence encoding the recombinant factor VIII polypeptide comprising an amino acid sequence comprising residues 20-1457 of SEQ ID NO: 252, 504, 706, 840, 1200, 1514, or 2072, or comprising SEQ ID NO: 252, 504, 706, 840, 1200, 1514, or 2072.

In some embodiments, the recombinant polynucleotide comprises a polynucleotide sequence encoding the full length polypeptide, or the pre-pro-protein or the pre-pro-polypeptide of the recombinant factor VIII polypeptide described herein.

In some embodiments, the recombinant polynucleotide comprises a polynucleotide sequence encoding the pro-protein or pro-polypeptide, or the mature form of the recombinant factor VIII polypeptide described herein.

In some embodiments, the recombinant polynucleotide comprises a polynucleotide sequence encoding the biologically or functionally active fragment of a recombinant VIII polypeptide described herein. In some embodiments, the recombinant polynucleotide comprises a polynucleotide sequence encoding the biologically or functionally active fragment having a cleavage or cleavages between amino acid residues 760 to 773, and the polypeptide being devoid of some or all of an amino acid sequence corresponding to amino acids 760-773, wherein the positions are relative to SEQ ID NO: 2, 252, 504, 706, 840, 1200, 1514, or 2072. In some embodiments, the recombinant polynucleotide comprises a polynucleotide sequence encoding the biologically or functionally active fragment having a cleavage between amino acid residues 391 and 392, wherein the positions are relative to the reference sequence of SEQ ID NO: 2, 252, 504, 706, 840, 1200, 1514, or 2072.

In some embodiments, the recombinant polynucleotide comprises a polynucleotide sequence having at least 70%, 75%, 80%, 81%, 82%, 83%, 84%, 85%, 86%, 87%, 88%, 89%, 90%, 91%, 92%, 93%, 94%, 95%, 96%, 97%, 98%, or 99% or more sequence identity to a reference polynucleotide sequence corresponding to nucleotide residues 58-4371 of an odd-numbered SEQ ID NO. of SEQ ID NOs: 3-2345 and 2359-2869, or to a reference polynucleotide sequence corresponding to an odd-numbered SEQ ID NO. of SEQ ID NOs: 3-2345 and 2349-2869, wherein the recombinant polynucleotide encodes a recombinant factor VIII polypeptide.

In some embodiments, the recombinant polynucleotide comprises a polynucleotide sequence having at least 70%, 75%, 80%, 81%, 82%, 83%, 84%, 85%, 86%, 87%, 88%, 89%, 90%, 91%, 92%, 93%, 94%, 95%, 96%, 97%, 98%, or 99% or more sequence identity to a reference polynucleotide sequence corresponding to nucleotide residues 58-4371 of SEQ ID NO: 1, 251, 503, 705, 839, 1199, 1513, or 2071, or to a reference polynucleotide sequence corresponding to SEQ ID NO: 1, 251, 503, 705, 839, 1199, 1513, or 2071, wherein the recombinant polynucleotide encodes a recombinant factor VIII polypeptide.

In some embodiments, the recombinant polynucleotide comprises a polynucleotide sequence encoding a recombinant factor VIII polypeptide described herein, wherein the polynucleotide sequence is codon-optimized for expression of the encoded recombinant factor VIII polypeptide. In some embodiments, the polynucleotide sequence is codon optimized for expression in prokaryotic cells or eukaryotic cells. In some embodiments, the polynucleotide sequence is codon optimized for expression in mammalian cells. In some embodiments, the polynucleotide sequence is codon optimized for expression in human cells.

In some embodiments, the recombinant polynucleotide comprises a polynucleotide sequence comprising nucleotide residues 58-4371 of an odd-numbered SEQ ID NO. of SEQ ID NOs: 3-2345 and 2359-2869, or comprising an odd-numbered SEQ ID NO. of SEQ ID NOs: 3-2345 and 2349-2869.

In some embodiments, the recombinant polynucleotide comprises a polynucleotide sequence comprising nucleotide residues 58-4371 of SEQ ID NO: 1, 251, 503, 705, 839, 1199, 1513, or 2071, or comprising SEQ ID NO. 1, 251, 503, 705, 839, 1199, 1513, or 2071.

In some embodiments, the present disclosure provides a recombinant polynucleotide capable of hybridizing under highly stringent conditions to a reference polynucleotide encoding a recombinant factor VIII described herein, e.g., a recombinant polynucleotide provided in Tables 4.1, 4.2, 5.1, 5.2, 6.1, 6.2, 7.1, 7.2, 8.1, 8.2, 9.1, 9.2, 10.1, 10.2, 11.1, and 15.1, or a reverse complement thereof. In some embodiments, the present disclosure provides a recombinant polynucleotide capable of hybridizing under highly stringent conditions to a reverse complement of a reference polynucleotide encoding a recombinant factor VIII polypeptide described herein, wherein the recombinant polynucleotide hybridizing under stringent conditions encodes a recombinant factor VIII polypeptide comprising an amino acid sequence having one or more residue differences as compared to SEQ ID NO: 2, 252, 504, 706, 840, 1200, 1514, or 2072 at residue positions selected from any positions as set forth in Tables 4.1, 4.2, 5.1, 5.2, 6.1, 6.2, 7.1, 7.2, 8.1, 8.2, 9.1, 9.2, 10.1, 10.2, 11.1, and 15.1.

In some embodiments, the recombinant polynucleotide that hybridizes under highly stringent conditions comprises a polynucleotide sequence having at least 70%, 75%, 80%, 81%, 82%, 83%, 84%, 85%, 86%, 87%, 88%, 89%, 90%, 91%, 92%, 93%, 94%, 95%, 96%, 97%, 98%, 99% or more sequence identity to a reference polynucleotide sequence corresponding to nucleotide residues nucleotide residues 58-4371 of SEQ ID NO: 1, 251, 503, 705, 839, 1199, 1513, or 2071, or to a reference sequence corresponding to SEQ ID NO: SEQ ID NO: 1, 251, 503, 705, 839, 1199, 1513, or 2071. In some additional embodiments, the recombinant polynucleotide hybridizing under highly stringent conditions comprises a polynucleotide sequence having at least 75%, 80%, 81%, 82%, 83%, 84%, 85%, 86%, 87%, 88%, 89%, 90%, 91%, 92%, 93%, 94%, 95%, 96%, 97%, 98%, 99% or more sequence identity to at least one polynucleotide reference sequence corresponding to nucleotide residues 58-4371 of a polynucleotide sequence provided in Tables 4.1, 4.2, 5.1, 5.2, 6.1, 6.2, 7.1, 7.2, 8.1, 8.2, 9.1, 9.2, 10.1, 10.2, 11.1, and 15.1. or a polynucleotide sequence provided in Tables 4.1, 4.2, 5.1, 5.2, 6.1, 6.2, 7.1, 7.2, 8.1, 8.2, 9.1, 9.2, 10.1, 10.2, 11.1, and 15.1. wherein the recombinant polynucleotide hybridizing under stringent conditions encodes a recombinant factor VIII polypeptide.

In some embodiments, an isolated polynucleotide encoding any of the recombinant factor VIII polypeptides provided herein is manipulated in a variety of ways to provide for expression of the polypeptide. In some embodiments, the polynucleotides encoding the polypeptides are provided as expression vectors where one or more control sequences is present to regulate the expression of the polynucleotides and/or polypeptides. Manipulation of the isolated polynucleotide prior to its insertion into a vector may be desirable or necessary depending on the expression vector. The techniques for modifying polynucleotides and nucleic acid sequences utilizing recombinant DNA methods are well known in the art.

In some embodiments, the control sequences include among others, promoter sequences, Kozak sequence, leader sequences, polyadenylation sequences, pro-peptide sequences, signal peptide sequences, DNA based regulatory elements for gene therapy retention and transcription terminators. As known in the art, suitable promoters can be selected based on the host cells used. Exemplary promoters for filamentous fungal host cells, include promoters obtained from the genes for Aspergillus oryzae TAKA amylase, Rhizomucor miehei aspartic proteinase, Aspergillus niger neutral alpha-amylase, Aspergillus niger acid stable alpha-amylase, Aspergillus niger or Aspergillus awamori glucoamylase (FVIIIA), Rhizomucor miehei lipase, Aspergillus oryzae alkaline protease, Aspergillus oryzae triose phosphate isomerase, Aspergillus nidulans acetamidase, and Fusarium oxysporum trypsin-like protease (See e.g., WO 96/00787), as well as the NA2-tpi promoter (a hybrid of the promoters from the genes for Aspergillus niger neutral alpha-amylase and Aspergillus oryzae triose phosphate isomerase), and mutant, truncated, and hybrid promoters thereof. Exemplary yeast cell promoters can be from the genes for Saccharomyces cerevisiae enolase (ENO-1), Saccharomyces cerevisiae galactokinase (GAL1), Saccharomyces cerevisiae alcohol dehydrogenase/glyceraldehyde-3-phosphate dehydrogenase (ADH2/GAP), and Saccharomyces cerevisiae 3-phosphoglycerate kinase. Other useful promoters for yeast host cells are known in the art (see e.g., Romanos et al., 1992, Yeast 8:423-488). Exemplary promoters for use in mammalian cells include, but are not limited to those from cytomegalovirus (CMV), chicken @-actin promoter fused with the CMV enhancer, Simian vacuolating virus 40 (SV40), from Homo sapiens phosphoglycerate kinase, beta actin, elongation factor-la or glyceraldehyde-3-phosphate dehydrogenase, or from Gallus @-actin.

In some embodiments, the control sequence is a suitable transcription terminator sequence, a sequence recognized by a host cell to terminate transcription. The terminator sequence is operably linked to the 3′ terminus of the nucleic acid sequence encoding the polypeptide. Any terminator which is functional in the host cell of choice finds use in the present invention. For example, exemplary transcription terminators for filamentous fungal host cells can be obtained from the genes for Aspergillus oryzae TAKA amylase, Aspergillus niger glucoamylase, Aspergillus nidulans anthranilate synthase, Aspergillus niger alpha-glucosidase, and Fusarium oxysporum trypsin-like protease. Exemplary terminators for yeast host cells can be obtained from the genes for Saccharomyces cerevisiae enolase, Saccharomyces cerevisiae cytochrome C (CYC1), and Saccharomyces cerevisiae glyceraldehyde-3-phosphate dehydrogenase. Other useful terminators for yeast host cells are known in the art (See e.g., Romanos et al., supra). Exemplary terminators for mammalian cells include, but are not limited to those from cytomegalovirus (CMV), Simian vacuolating virus 40 (SV40), from Homo sapiens growth hormone hGH, from bovine growth hormone BGH, and from human or rabbit beta globulin.

In some embodiments, the control sequence is a suitable leader sequence, 5′-cap modification, 5′ UTR, etc. In some embodiments, these regulatory sequence elements mediate binding to molecules involved in mRNA trafficking and translation, inhibit 5′-exonucleolytic degradation and confer resistance to de-capping. The leader sequence is operably linked to the 5′ terminus of the nucleic acid sequence encoding the polypeptide. Any leader sequence that is functional in the host cell of choice may be used. Exemplary leaders for filamentous fungal host cells are obtained from the genes for Aspergillus oryzae TAKA amylase and Aspergillus nidulans triose phosphate isomerase. Suitable leaders for yeast host cells include, but are not limited to those obtained from the genes for Saccharomyces cerevisiae enolase (ENO-1), Saccharomyces cerevisiae 3-phosphoglycerate kinase, Saccharomyces cerevisiae alpha-factor, and Saccharomyces cerevisiae alcohol dehydrogenase/glyceraldehyde-3-phosphate dehydrogenase (ADH2/GAP). Suitable leaders for mammalian host cells include but are not limited to the 5′-UTR element present in orthopoxvirus mRNA.

In some embodiments, the control sequence comprises a 3′ untranslated nucleic acid region and polyadenylation tail nucleic acid sequence, sequences operably linked to the 3′ terminus of the protein coding nucleic acid sequence which mediate binding to proteins involved in mRNA trafficking and translation and mRNA half-life. Any polyadenylation sequence and 3′ UTR which is functional in the host cell of choice may be used in the present invention. Exemplary polyadenylation sequences for filamentous fungal host cells include, but are not limited to those from the genes for Aspergillus oryzae TAKA amylase, Aspergillus niger glucoamylase, Aspergillus nidulans anthranilate synthase, Fusarium oxysporum trypsin-like protease, and Aspergillus niger alpha-glucosidase. Useful polyadenylation sequences for yeast host cells are also known in the art (See e.g., Guo and Sherman, Mol. Cell. Biol., 1995, 15:5983-5990). Useful polyadenylation and 3′ UTR sequences for mammalian host cells include, but are not limited to, the 3′-UTRs of α- and β-globin mRNAs that harbor several sequence elements that increase the stability and translation of mRNA.

In some embodiments, the control sequence is a signal peptide coding region that codes for an amino acid sequence linked to the amino terminus of a polypeptide and directs the encoded polypeptide into the cell's secretory pathway. The 5′ end of the coding sequence of the nucleic acid sequence may inherently contain a signal peptide coding region naturally linked in translation reading frame with the segment of the coding region that encodes the secreted polypeptide. Alternatively, the 5′ end of the coding sequence may contain a signal peptide coding region that is foreign to the coding sequence. Any signal peptide coding region that directs the expressed polypeptide into the secretory pathway of a host cell of choice finds use for expression of the recombinant factor VIII polypeptides provided herein. Effective signal peptide coding regions for filamentous fungal host cells include, but are not limited to the signal peptide coding regions obtained from the genes for Aspergillus oryzae TAKA amylase, Aspergillus niger neutral amylase, Aspergillus niger glucoamylase, Rhizomucor miehei aspartic proteinase, Humicola insolens cellulase, and Humicola lanuginosa lipase. Useful signal peptides for yeast host cells include, but are not limited to those from the genes for Saccharomyces cerevisiae alpha-factor and Saccharomyces cerevisiae invertase. Useful signal peptides for mammalian host cells include but are not limited to those from the genes for immunoglobulin gamma (IgG). In some embodiments, the signal peptide is a signal peptide of a protein expressed in human cells.

In another aspect, the present disclosure also provides a recombinant expression vector comprising a polynucleotide encoding a recombinant factor VIII polypeptide, and one or more expression regulating regions such as a promoter and a terminator, a replication origin, etc., depending on the type of hosts into which they are to be introduced. In some embodiments, the various nucleic acid and control sequences described above are joined together to produce a recombinant expression vector which includes one or more convenient restriction sites to allow for insertion or substitution of the nucleic acid sequence encoding the recombinant factor VIII polypeptide at such sites. Alternatively, the polynucleotide sequence(s) of the present disclosure are expressed by inserting the polynucleotide sequence or a nucleic acid construct comprising the polynucleotide sequence into an appropriate vector for expression. In creating the expression vector, the coding sequence is located in the vector so that the coding sequence is operably linked with the appropriate control sequences for expression.

The recombinant expression vector may be any vector (e.g., a plasmid or virus including but not limited to adenovirus (AV), adeno-associated virus (AAV), lentivirus (LV), and non-viral vectors, such as liposomes), that can be conveniently subjected to recombinant DNA procedures and can result in the expression of the recombinant factor VIII polynucleotide sequence. The choice of the vector will typically depend on the compatibility of the vector with the host cell into which the vector is to be introduced. The vectors may be linear or closed circular plasmids.

In some embodiments, the expression vector is an autonomously replicating vector (i.e., a vector that exists as an extra-chromosomal entity, the replication of which is independent of chromosomal replication, such as a plasmid, an extra-chromosomal element, a minichromosome, or an artificial chromosome). The vector may contain any means for assuring self-replication. In some alternative embodiments, the vector may be one which, when introduced into the host cell, is integrated into the genome and replicated together with the chromosome(s) into which it has been integrated. Furthermore, a single vector or plasmid or two or more vectors or plasmids which together contain the total DNA to be introduced into the genome of the host cell, or a transposon may be used.

In some embodiments, the expression vector preferably contains one or more selectable markers, which permit easy selection of transformed cells. A “selectable marker” is a gene the product of which provides for biocide or viral resistance, resistance to heavy metals, prototrophy to auxotrophs, and the like. Suitable markers for yeast host cells include, but are not limited to ADE2, HIS3, LEU2, LYS2, MET3, TRP1, and URA3. Selectable markers for use in a filamentous fungal host cell include, but are not limited to, amdS (acetamidase), argB (ornithine carbamoyltransferases), bar (phosphinothricin acetyltransferase), hph (hygromycinphosphotransferase), niaD (nitrate reductase), pyrG (orotidine-5′-phosphate decarboxylase), sC (sulfate adenyltransferase), and trpC (anthranilate synthase), as well as equivalents thereof.

In another aspect, the present invention provides a host cell comprising a polynucleotide encoding at least one recombinant factor VIII polypeptide described herein, the polynucleotide being operatively linked to one or more control sequences for expression of the recombinant factor VIII polypeptides(s) in the host cell. Host cells for use in expressing the polypeptides encoded by the expression vectors of the present invention are well known in the art and include but are not limited to, bacterial cells, fungal cells, such as yeast cells (e.g., Saccharomyces cerevisiae and Pichia pastoris, e.g., ATCC Accession No. 201178); insect cells (e.g., Drosophila S2 and Spodoptera Sf9 cells), plant cells, animal cells (e.g., CHO, CHO-K1, COS, and BHK), and human cells (e.g., HEK293T, human fibroblast, THP-1, Jurkat and Bowes melanoma cell lines). In some embodiments, the host cell is a human cell obtained or derived from a subject with hemophilia A.

Accordingly, in another aspect, the present disclosure provides a method for producing the recombinant factor VIII polypeptides, where the method comprises culturing a host cell capable of expressing a polynucleotide encoding the recombinant factor VIII polypeptide under conditions suitable for expression of the polypeptide. In some embodiments, the method further comprises the step of isolating the recombinant factor VIII polypeptide, such as from the culture media or cells. In some embodiments, the method further comprises the step of purifying the expressed recombinant factor VIII polypeptide, as described herein.

Appropriate culture media and growth conditions for the above-described host cells are well known in the art. Polynucleotides for expression of the recombinant factor VIII polypeptides may be introduced into cells by various methods known in the art. Techniques include, among others, electroporation, biolistic particle bombardment, liposome mediated transfection, calcium chloride transfection, and protoplast fusion.

The recombinant factor VIII polypeptides with the properties disclosed herein can be obtained by subjecting the polynucleotide encoding the naturally occurring or recombinant factor VIII polypeptide to mutagenesis and/or directed evolution methods known in the art, and as described herein. An exemplary directed evolution technique is mutagenesis and/or DNA shuffling (See e.g., Stemmer, Proc. Natl. Acad. Sci. USA, 1994, 91:10747-10751; WO 95/22625; WO 97/0078; WO 97/35966; WO 98/27230; WO 00/42651; WO 01/75767 and U.S. Pat. No. 6,537,746). Other directed evolution procedures that can be used include, among others, staggered extension process (StEP), in vitro recombination (See e.g., Zhao et al., Nat. Biotechnol., 1998, 16:258-261), mutagenic PCR (See e.g., Caldwell et al., PCR Methods Appl., 1994, 3:S136-S140), and cassette mutagenesis (see e.g., Black et al., Proc. Natl. Acad. Sci. USA, 1996, 93:3525-3529).

For example, mutagenesis and directed evolution methods can be readily applied to polynucleotides to generate variant libraries that can be expressed, screened, and assayed. Mutagenesis and directed evolution methods are well known in the art (See e.g., U.S. Pat. Nos. 5,605,793, 5,811,238, 5,830,721, 5,834,252, 5,837,458, 5,928,905, 6,096,548, 6,117,679, 6,132,970, 6,165,793, 6,180,406, 6,251,674, 6,277,638, 6,287,861, 6,287,862, 6,291,242, 6,297,053, 6,303,344, 6,309,883, 6,319,713, 6,319,714, 6,323,030, 6,326,204, 6,335,160, 6,335,198, 6,344,356, 6,352,859, 6,355,484, 6,358,740, 6,358,742, 6,365,377, 6,365,408, 6,368,861, 6,372,497, 6,376,246, 6,379,964, 6,387,702, 6,391,552, 6,391,640, 6,395,547, 6,406,855, 6,406,910, 6,413,745, 6,413,774, 6,420,175, 6,423,542, 6,426,224, 6,436,675, 6,444,468, 6,455,253, 6,479,652, 6,482,647, 6,489,146, 6,506,602, 6,506,603, 6,519,065, 6,521,453, 6,528,311, 6,537,746, 6,573,098, 6,576,467, 6,579,678, 6,586,182, 6,602,986, 6,613,514, 6,653,072, 6,716,631, 6,946,296, 6,961,664, 6,995,017, 7,024,312, 7,058,515, 7,105,297, 7,148,054, 7,288,375, 7,421,347, 7,430,477, 7,534,564, 7,620,500, 7,620,502, 7,629,170, 7,702,464, 7,747,391, 7,747,393, 7,751,986, 7,776,598, 7,783,428, 7,795,030, 7,853,410, 7,868,138, 7,873,499, 7,904,249, 7,957,912, 8,383,346, 8,504,498, 8,849,575, 8,876,066, 8,768,871, 9,593,326, and all related non-US counterparts; Ling et al., Anal. Biochem., 1997, 254(2):157-78; Dale et al., Meth. Mol. Biol., 1996, 57:369-74; Smith, Ann. Rev. Genet, 1985, 19:423-462; Botstein et al., Science, 1985, 229:1193-1201; Carter, Biochem. J., 1986, 237:1-7; Kramer et al., 1984, Cell, 38:879-887; Wells et al., Gene, 1985, 34:315-323; Minshull et al., Curr. Op. Chem. Biol., 1999, 3:284-290; Christians et al., Nat. Biotechnol., 1999, 17:259-264; Crameri et al., Nature, 1998, 391:288-291; Crameri, et al., Nat. Biotechnol., 1997, 15:436-438; Zhang et al., Proc. Nat. Acad. Sci. USA, 1997, 94:4504-4509; Crameri et al., Nat. Biotechnol., 1996, 14:315-319; Stemmer, Nature, 1994, 370:389-391; Stemmer, Proc. Nat. Acad. Sci. USA, 1994, 91:10747-10751; U.S. patent publication Nos. 2008/0220990, US 2009/0312196, US2014/0005057, US2014/0214391, US2014/0221216; US2015/0050658, US2015/0133307, US2015/0134315 and all related non-US counterparts; WO 95/22625, WO 97/0078, WO 97/35966, WO 98/27230, WO 00/42651, WO 01/75767, and WO 2009/152336; all of which are incorporated herein by reference).

In some embodiments, the protein variants obtained following mutagenesis treatment are screened by subjecting the protein variants to a defined temperature, pH, or other assay conditions, and measuring the amount of protein activity remaining after the treatments or other assay conditions. DNA containing the polynucleotide encoding the factor VIII polypeptide is then isolated from the host cell, sequenced to identify the nucleotide sequence changes (if any), and used to express the protein in a different or the same host cell. Measuring protein activity from the expression libraries can be performed using any suitable method known in the art, such as provided in the Examples.

For engineered polypeptides of known sequence, the polynucleotides encoding the polypeptide can be prepared by standard solid-phase methods, according to known synthetic methods. In some embodiments, polynucleotide fragments can be individually synthesized, then joined (e.g., by enzymatic or chemical litigation methods, or polymerase mediated methods) to form any desired continuous sequence. For example, polynucleotides and oligonucleotides disclosed herein can be prepared by chemical synthesis using the classical phosphoramidite method (See e.g., Beaucage et al., Tetra. Lett., 1981, 22:1859-69; and Matthes et al., EMBO J., 1984, 3:801-05), as it is typically practiced in automated synthetic methods.

Accordingly, in some embodiments, a method for preparing the recombinant factor VIII polypeptide can comprise: (a) synthesizing a polynucleotide encoding a polypeptide comprising an amino acid sequence selected from the amino acid sequence of any variant provided in Tables 4.1, 4.2, 5.1, 5.2, 6.1, 6.2, 7.1, 7.2, 8.1, 8.2, 9.1, 9.2, 10.1, 10.2, 11.1, and 15.1, as well as SEQ ID NOs: 2-2346 and 2350-2870, and (b) expressing the recombinant factor VIII polypeptide encoded by the polynucleotide. In some embodiments of the method, the amino acid sequence encoded by the polynucleotide can optionally have one or several (e.g., up to 3, 4, 5, or up to 10) amino acid residue deletions, insertions and/or substitutions. In some embodiments, the amino acid sequence has optionally 1-2, 1-3, 1-4, 1-5, 1-6, 1-7, 1-8, 1-9, 1-10, 1-15, 1-20, 1-21, 1-22, 1-23, 1-24, 1-25, 1-30, 1-35, 1-40, 1-45, or 1-50 amino acid residue deletions, insertions and/or substitutions. In some embodiments, the amino acid sequence has optionally 1, 2, 3, 4, 5, 6, 7, 8, 9, 10, 11, 12, 13, 14, 15, 16, 17, 18, 19, 20, 21, 22, 23, 24, 25, 30, 30, 35, 40, 45, or 50 amino acid residue deletions, insertions and/or substitutions. In some embodiments, the amino acid sequence has optionally 1, 2, 3, 4, 5, 6, 7, 8, 9, 10, 11, 12, 13, 14, 15, 16, 18, 20, 21, 22, 23, 24, or 25 amino acid residue deletions, insertions and/or substitutions. In some embodiments, the substitutions can be conservative or non-conservative substitutions.

The expressed recombinant factor VIII polypeptide can be assessed for any desired improved property (e.g., activity/potency, selectivity, stability, serum/plasma stability, basic pH tolerance, protease sensitivity, etc.), using any suitable assay known in the art, including but not limited to the assays and conditions described herein.

In some embodiments, any of the recombinant factor VIII polypeptides expressed in a host cell are recovered from the cells and/or the culture medium using any one or more of the well-known techniques for protein purification, including, among others, lysozyme or detergent treatment, sonication, filtration, salting-out, ultra-centrifugation, and chromatography.

Chromatographic techniques for isolation of the recombinant factor VIII polypeptides include, among others, reverse phase chromatography high performance liquid chromatography, ion exchange chromatography, hydrophobic interaction chromatography, gel electrophoresis, and affinity chromatography. Conditions for purifying a particular polypeptide depends, in part, on factors such as net charge, hydrophobicity, hydrophilicity, molecular weight, molecular shape, etc., and will be apparent to those having skill in the art. In some embodiments, affinity techniques may be used to isolate the improved variant factor VIII polypeptides. In some embodiments utilizing affinity chromatography purification, any antibody which specifically binds the variant factor VIII polypeptide finds use. In some embodiments utilizing affinity chromatography purification, proteins that bind to the glycans covalently attached to factor VIII find use. In still other embodiments utilizing affinity-chromatography purifications, any small molecule that binds to the factor VIII active site finds use. For the production of antibodies, various host animals, including but not limited to rabbits, mice, rats, etc., are immunized by injection with a factor VIII polypeptide (e.g., a factor VIII variant), or a fragment thereof. In some embodiments, the factor VIII polypeptide or fragment is attached to a suitable carrier, such as BSA, by means of a side chain functional group or linkers attached to a side chain functional group.

In some embodiments, once the recombinant factor VIII polypeptides are recovered or isolated from the recombinant host cells or cell culture medium, they are further purified by any suitable method(s) known in the art. In some additional embodiments, the purified factor VIII polypeptides are combined with other ingredients and compounds to provide compositions and formulations comprising the recombinant factor VIII polypeptide as appropriate for different applications and uses (e.g., pharmaceutical compositions).

In some additional embodiments, the purified recombinant factor VIII polypeptides, or the formulated recombinant factor VIII polypeptides are lyophilized. In some embodiments, the recombinant factor VIII polypeptides are directly produced within a body (i.e., cells within a body, such as a human or another animal) and are not purified. However, in some alternative embodiments, the recombinant factor VIII polypeptides are produced within a body (i.e., cells within a body, such as a human or another animal) and are collected from the body using methods known in the art. In some additional embodiments, these collected recombinant factor VIII polypeptides are purified.

### Pharmaceutical Compositions

In a further aspect, the present disclosure provides various compositions and formats comprising a recombinant factor VIII polypeptide, including but not limited to those described below. In some embodiments, the present disclosure provides recombinant polypeptides or recombinant polynucleotides suitable for use in pharmaceutical and other compositions.

Depending on the mode of administration, a composition comprising a therapeutically effective amount of a recombinant factor VIII described herein is in the form of a solid, semi-solid, or liquid. In some embodiments, the composition includes other pharmaceutically acceptable components such as diluents, buffers, excipients, salts, emulsifiers, preservatives, stabilizers, fillers, and other ingredients. Details on techniques for formulation and administration are well known in the art and described in the literature. In some embodiments, these compositions are produced directly in the human body after introduction as gene therapy.

In some embodiments, the present disclosure provides a pharmaceutical composition comprising a recombinant factor VIII polypeptide described herein. In some embodiments, the pharmaceutical composition further comprises a pharmaceutically acceptable carrier or excipient. Any suitable form for use in delivering the recombinant factor VIII polypeptides find use in the present disclosure, including but not limited to pills, tablets, gel tabs, capsules, lozenges, dragees, powders, soft gels, sol-gels, gels, emulsions, implants, patches, sprays, ointments, liniments, creams, pastes, jellies, paints, aerosols, chewing gums, demulcents, sticks, solutions, suspensions (including but not limited to oil-based suspensions, oil-in water emulsions, etc.), slurries, syrups, controlled release formulations, etc.

In some embodiments, the recombinant factor VIII polypeptides are provided in a form suitable for injection or infusion (i.e., in an injectable formulation). In some embodiments, the polynucleotide sequences for the recombinant factor VIII polypeptide is provided in a form suitable for injection or infusion.

In some embodiments, the recombinant factor VIII polypeptides are provided in biocompatible matrices such as sol-gels, including silica-based (e.g., oxysilane) sol-gels. In some embodiments, the recombinant factor VIII polypeptides are encapsulated. In some alternative embodiments, the recombinant factor VIII polypeptides are encapsulated in nanostructures (e.g., nanotubes, nanotubules, nanocapsules, or microcapsules, microspheres, liposomes, etc.).

In some embodiments, a composition comprising the recombinant factor VIII polypeptides of the present disclosure may include one or more commonly used carrier compounds, including but not limited to sugars (e.g., lactose, sucrose, mannitol, and/or sorbitol), starches, cellulose (e.g., methyl cellulose, hydroxypropylmethyl cellulose, sodium carboxy-methylcellulose), gums (e.g., arabic, tragacanth, guar, etc.), and/or proteins (e.g., gelatin, collagen, etc.).

In some embodiments, the recombinant factor VIII polypeptides are chemically modified by glycosylation, chemical crosslinking reagents, pegylation (i.e., modified with polyethylene glycol (PEG) or activated PEG, etc.) or other compounds (see e.g., Ikeda, Amino Acids, 2005, 29:283-287; U.S. Pat. Nos. 7,531,341, 7,534,595, and 7,560,263; US Pat. Publication Nos. 2013/0039898, 2012/0177722, etc.).

It is intended that the recombinant factor VIII polypeptides be administered by any suitable means known in the art, including but not limited to parenteral, oral, topical, transdermal, intranasal, intraocular, intrathecal, via implants, etc. It is not intended that the present invention be limited to any particular delivery method and/or mechanism.

In some embodiments, the pharmaceutical composition comprises a recombinant polynucleotide encoding a recombinant factor VIII polynucleotide described herein, such as for gene therapy. In some embodiments, a gene therapy composition comprises a recombinant polynucleotide encoding any of the recombinant factor VIII polypeptides described herein. In some embodiments, the gene therapy composition comprises a viral vector comprising the recombinant polynucleotide encoding a recombinant factor VIII polypeptide. In some embodiments, viral delivery vectors, include but are not limited to adenovirus (AV), adeno-associated virus (AAV), lentivirus (LV), retrovirus, or herpes virus vectors. In some embodiments, the gene therapy compositions comprise non-viral delivery compositions, where the recombinant polynucleotide is formulated with liposomes, cationic polymers, dendrimers, or conjugated to cell-penetrating peptides. In some embodiments, the recombinant factor VIII polypeptides are provided for delivery to cells or tissues via mRNA therapy, such as by formulating a polyribonucleotide (e.g., mRNA) in an encapsulated delivery vehicle (e.g., liposomes).

In some additional embodiments, the recombinant factor VIII polypeptides are provided for delivery to cells or tissues via cell therapy, where the polynucleotide sequence encoding the recombinant factor VIII polypeptides is introduced into exogenous cell and that cell (or cells) are introduced into a recipient or subject (e.g., a patient exhibiting deficiency in factor VIII). In some embodiments, the cell is an stem cell, particularly a human stem cell.

### Uses and Methods

In another aspect, the recombinant factor VIII polypeptides disclosed herein are used for therapeutic purposes. In some embodiments, the recombinant factor VIII polypeptides and/or recombinant polynucleotides encoding the recombinant factor VIII polypeptides are used in the treatment of conditions or diseases associated with a deficiency in factor VIII.

In some embodiments, a method for treating and/or preventing the symptoms of deficiency in factor VIII in a subject comprises administering to a subject having a deficiency in factor VIII a therapeutically effective amount of a recombinant factor VIII polypeptide described herein, or a pharmaceutical composition thereof. In some embodiments, administration of the recombinant factor VIII is provided as enzyme replacement therapy.

In some embodiments, a method for treating and/or preventing the symptoms of deficiency in factor VIII in a subject comprises providing to a subject having a deficiency in factor VIII a recombinant polynucleotide encoding a recombinant factor VIII polypeptide, such as the gene therapy composition comprising the recombinant polypeptide described above. In some embodiments, the recombinant polynucleotides are provided in a viral vector. In some embodiments, the viral vectors are expression vectors that provide for expression of the encoded recombinant factor VIII polypeptide when the vector is administered to a patient or inserted in a mammalian cell. In some embodiments, the viral vector is based on adenovirus, adeno-associated virus, lentivirus, retrovirus, or herpes virus vectors. In some embodiments, the recombinant polynucleotide is formulated in non-viral delivery compositions, as described above. In some embodiments, the recombinant polynucleotide is provided as gene replacement therapy, for example using CRISPR technology.

In some embodiments, the condition or disease associated with a deficiency in factor VIII and treated with the recombinant factor VIII polypeptide or a polynucleotide encoding the recombinant factor VIII polypeptide is hemophilia A. In some embodiments, the hemophilia is acquired hemophilia A. In some embodiments, the hemophilia A is from a genetic defect in the gene encoding factor VIII. In some embodiments, the symptoms of deficiency in factor VIII are ameliorated by the treatment.

### EXAMPLES

The following Examples, including experiments and results achieved, are provided for illustrative purposes only and are not to be construed as limiting the present invention.

In the experimental disclosure below, the following abbreviations apply where applicable: ppm (parts per million); M (molar); mM (millimolar), uM and μM (micromolar); nM (nanomolar); mol (moles); gm and g (gram); mg (milligrams); ug and μg (micrograms); L and l (liter); ml and mL (milliliter); cm (centimeters); mm (millimeters); um and μm (micrometers); sec. (seconds); min(s) (minute(s)); h(s) and hr(s) (hour(s)); U (units); MW (molecular weight); rpm (rotations per minute); ° C. (degrees Centigrade); CDS (coding sequence); DNA (deoxyribonucleic acid); RNA (ribonucleic acid); FIOPC (fold improvements over positive control).

### Example 1

### FVIII Gene Acquisition and Construction of Expression Vectors

Synthetic genes (SEQ ID NO: 1) coding for the WT human FVIII sequence with B-domain deleted (SEQ ID NO: 2) were constructed as previously described (See e.g. U.S. Patent Publication. No. 2017/0360900). Oligonucleotides BamHI-pcDNA-FVIII-F and XhoI-pcDNA-FVIII-R were used to amplify a fragment for the coding sequence for the FVIII variants. The PCR product was ligated into the BamHI/XhoI linearized mammalian expression vector pcDNA3.1(+) (Invitrogen), or a vector containing a CMV promoter and BGH-pA (bovine growth hormone polyadenylation) sequence. Directed evolution techniques generally known by those skilled in the art were used to generate gene variants derived from SEQ ID NO: 2 within this plasmid construct (see e.g., U.S. Pat. No. 8,383,346 and International patent publications WO2010/144103).

Secreted expression of a mammalian cell codon-optimized human FVIII was achieved using the native FVIII signal peptide. Clones were expressed primarily from a pDH.1(+) vector in HEK293T cells, Expi293F™ (ThermoFisher Scientific) cells, and HepG2 cells. This approach provided supernatants with measurable activity in the chromogenic assay (See e.g., Aniara Diagnostica BIOPHEN™ FVIII:C) and the activated partial thromboplastin time (APTT) assay, using plasma from hemophilia A patients.

In some embodiments, to identify mutational diversity with similar stability and improved blood clotting function compared to SEQ ID NO: 2, a combinatorial library was constructed generating FVIII variants derived from SEQ ID NO:2. Equivalent volumes of supernatant were screened in an unchallenged condition (no incubation, pH 7.4) or following a one-hour to seventeen-hour incubation in a human plasma (physiological pH 7.1-8.2) environment at 37° C. to 55° C. FVIII variants with improved activity due to increased FVIII expression or FVIII specific activity were identified based on their fold improvement over the parent FVIII. FVIII variants with increased stability were identified by dividing the fold-improvement observed under challenged conditions by the fold-improvement observed under unchallenged conditions. This approach reduces the bias towards selecting variants based on increased expression but without changes in stability. Composite activity scores (the product of fold-improvements for all three conditions) and stability (the product of stability scores) were used to rank mutations in improved variants for inclusion in subsequent FVIII libraries. In additional embodiments, the additional methods and sequences described in the Examples were used.

### Example 2

### High-Throughput Growth

### High-Throughput (HTP) Growth of FVIII and FVIII Variants

Expi293F™ (ThermoFisher Scientific) cells or HepG2 cells were transfected with pcDNA 3.1(+) having a CMV promoter and BGH-pA containing vectors encoding wild type FVIII with B-domain deleted, or variants, using the lipofection method with ExpiFectamine™ 293 Transfection Kit (ThermoFisher Scientific) or LIPOFECTAMINE® 3000 Reagent (ThermoFisher Scientific). Expi293F™ (ThermoFisher Scientific) cells were cultured in growth medium (Expi293™ Expression Medium, ThermoFisher Scientific). Immediately before transfection, cells were transferred to NUNC™ 96-Well DeepWell™ plates (ThermoFisher Scientific) at densities of 10⁸ cells/well/400 μL in growth medium, and incubated at 37° C., 8% CO₂ in an incubator. HepG2 cells were cultured in growth medium (EMEM with 10% fetal bovine serum, both from Corning). 24 hours before transfection, cells were seeded to NUNC® Edge 2.0 96-well plate (ThermoFisher Scientific) at densities of 10⁴ cells/well/250 μL in growth medium, and incubated at 37° C., 5% CO₂ in an incubator. Cells were incubated for 24-96 hours at specific conditions to allow for expression and secretion of FVIII variants. Conditioned media (50-100 μL) from the transfections were transferred into Corning 96-well solid black plates (Corning) for activity and/or stability analysis.

### Example 3

### In Vitro Characterization of Variants in HTP

### HTP-Analysis of Supernatants (Chromogenic Assay)

FVIII variant activity was determined by measuring the release of paranitroaniline (pNa) from a synthetic peptide substrate. For an unchallenged assay, 25 μL of Expi293F or HepG2 conditioned media produced as described above were warmed to 37° C. and mixed with 25 μL of “Reagent 1” (BIOPHEN™ FVIII:C, Aniara) and 25 μL of “Reagent 2” (BIOPHEN™ FVIII:C, Aniara) in a 96-well, black, opaque bottom microtiter plate. The reactions were mixed briefly and incubated at 37° C. for 5 minutes. 25 μL of “Reagent 3” (BIOPHEN™ FVIII:C, Aniara) was then added, and the reactions were mixed briefly and incubated at 37° C. for 5 minutes prior to quenching with 25 μL pf 2% citric acid. Release of pNA was analyzed using an EnVision® 2105 Multimode Plate Reader (PerkinElmer) monitoring absorbance at 405 nm. “Reagent 1”, “Reagent 2”, and “Reagent 3” are components of a chromogenic assay (BIOPHEN™ FVIII:C) supplied by Aniara and used as recommended by the supplier.

### HTP-Analysis of Supernatants (Clotting Assay)

FVIII variant activity was determined by measuring the clotting rate of Hemophilia A patient plasma (George King Bio-Medical, Inc.) using the activated partial thromboplastin time assay. For an unchallenged assay, 25 μL of Expi293F or HepG2 conditioned media produced as described above were warmed to 37° C. and mixed with 30 μL of Hemophilia A patient plasma (George King Bio-Medical, Inc.) and 50 μL of aPTT-XL reagent (Pacific Hemostasis) in a 96-well, black, opaque bottom microtiter plate. The reactions were mixed briefly and incubated at 37° C. for 3 minutes. 10 μL of 200 μM calcium chloride (Sigma Aldrich) was then added, and the reactions were mixed briefly. Clotting was analyzed using an EnVision® 2105 Multimode Plate Reader (PerkinElmer) monitoring absorbance at 405 nm over time for 5-10 minutes.

HTP-Analysis of Supernatants Pretreated with Plasma

FVIII variants were challenged with Hemophilia A patient plasma (George King Bio-Medical, Inc.) to simulate the conditions that the variants may encounter within the bloodstream. First, 50 μL of Expi293F or HepG2 conditioned media produced as described above and 50 μL of Hemophilia A patient plasma (George King Bio-Medical, Inc.) were added to the wells of a 96-well round bottom microtiter plate. The plates were sealed with a PlateLoc® thermal microplate sealer (Agilent), and incubated at 37-55° C. for 1-24 h. The samples were then analyzed by either the chromogenic or clotting assays described above.

### Example 4

### FVIII Variants Derived from SEQ ID NO: 2

In this Example, experiments conducted to determine FVIII variant activity are described. These variants were tested using the chromogenic assays, as described in Example 3. Results are shown in Tables 4.1 and 4.2 for variants expressed using Expi293F cells.

TABLE 4.1
Activity of FVIII Variants Relative to SEQ ID NO: 2

| | Amino Acid | Unchallenged Chromogenic |
|---|---|---|
| SEQ ID | Differences | Activity FIOPC |
| NO: | (Relative to | Relative to |
| (nt/aa) | SEQ ID NO: 2) | SEQ ID NO: 2 |
| 1/2 | | + |
| 3/4 | T1148N | + |
| 5/6 | P85Q/I708T/T1148N | ++ |
| 7/8 | K798T | + |
| 9/10 | L706G | + |
| 11/12 | L706A | + |
| 13/14 | L171Q | + |
| 15/16 | Q335E | + |
| 17/18 | V63R | ++ |
| 19/20 | K344A | ++ |
| 21/22 | S428F | ++ |
| 23/24 | E879Q | ++ |
| 25/26 | F59L | + |
| 27/28 | Q429S | + |
| 29/30 | K205R | ++ |
| 31/32 | I1264W | + |
| 33/34 | M797P | + |
| 35/36 | L1248S | + |
| 37/38 | I1264R | + |
| 39/40 | Y873T | + |
| 41/42 | T792Y | + |
| 43/44 | I708K | + |
| 45/46 | K344L | ++ |
| 47/48 | F57V | + |
| 49/50 | Q96L | + |
| 51/52 | A362W | + |
| 53/54 | V63G | ++ |
| 55/56 | Y873L | + |
| 57/58 | M1292Y | ++ |
| 59/60 | T1148Q | ++ |
| 61/62 | D346V | ++ |
| 63/64 | M797G | + |
| 65/66 | L706Y | + |
| 67/68 | L1248R | ++ |
| 69/70 | N433A | ++ |
| 71/72 | Y65A | + |
| 73/74 | F59W | + |
| 75/76 | Y873Q | + |
| 77/78 | V795C | + |
| 79/80 | E684K/K798R | ++ |
| 81/82 | F57L | + |
| 83/84 | Q353S | + |
| 85/86 | S332Q | + |
| 87/88 | I708A | + |
| 89/90 | G1259T | + |
| 91/92 | V63M | + |
| 93/94 | M356L | ++ |
| 95/96 | L1248G | ++ |
| 97/98 | L1248P | ++ |
| 99/100 | Q436G | + |
| 101/102 | F57A | + |
| 103/104 | F59S | ++ |
| 105/106 | T1245R | + |
| 107/108 | F59R | + |
| 109/110 | Q96W | + |
| 111/112 | M797Y | + |
| 113/114 | K1145R | + |
| 115/116 | Q436D | + |
| 117/118 | F716L | + |
| 119/120 | S56E | + |
| 121/122 | R773H | + |
| 123/124 | T734I | ++ |
| 125/126 | N358S | ++ |
| 127/128 | Q335P | + |
| 129/130 | F59Q | ++ |
| 131/132 | S729T | + |
| 133/134 | F716K | ++ |
| 135/136 | T665G | + |
| 137/138 | A362T | + |
| 139/140 | F57T | + |
| 141/142 | N359G | + |
| 143/144 | T61S | ++ |
| 145/146 | V1250A | + |
| 147/148 | A362V | + |
| 149/150 | N359E | + |
| 151/152 | F716W | + |
| 153/154 | S332W | ++ |
| 155/156 | I708V | + |
| 157/158 | V63H | ++ |
| 159/160 | M797C | + |
| 161/162 | T61Q | + |
| 163/164 | M356N | ++ |
| 165/166 | V1250S | + |
| 167/168 | K798C | + |
| 169/170 | N433G | ++ |
| 171/172 | V795N | ++ |
| 173/174 | Q96I | + |
| 175/176 | S56P | + |
| 177/178 | V63N | + |
| 179/180 | T792W | + |
| 181/182 | I1264L | + |
| 183/184 | V1250P | + |
| 185/186 | S332P | + |
| 187/188 | F59V | + |
| 189/190 | K726L | ++ |

All activities were determined relative to the reference polypeptide of SEQ ID NO: 2. Levels of increased activity are defined as follows: “+” 0.9 to 1.1, “++” >1.1

TABLE 4.2
Activity of FVIII Variants Relative to SEQ ID NO: 2

| | | Unchallenged |
|---|---|---|
| | Amino Acid | Chromogenic |
| SEQ ID | Differences | Activity FIOPC |
| NO: | (Relative to | Relative to |
| (nt/aa) | SEQ ID NO: 2) | SEQ ID NO: 2 |
| 1/2 | | + |
| 191/192 | Q37H/Q96R/L171P/Q429L/I708T/ | ++ |
| | K799R/T1148N | |
| 193/194 | L171P | ++ |
| 195/196 | L171P/Q429L/K732R/T1148N | ++ |
| 197/198 | Q96R/L171P | ++ |
| 199/200 | L171P/E919G/G1151S | +++ |
| 201/202 | L171P/T792Y/M797Q/K799R/T1148N | ++ |
| 203/204 | L171P/P569T/T1148N | ++ |
| 205/206 | Q96R/L171P/Q429L/T792Y/M797T/ | ++ |
| | K799R | |
| 207/208 | L171P/Q429L/I708T/L1422I | +++ |
| 209/210 | L171P/Q429L | ++ |
| 211/212 | D321Y/A362P/K732E/M797T/K799R | ++ |
| 213/214 | L171P/L1100I | ++ |
| 215/216 | I708T/T1148N/A1186E | +++++ |
| 217/218 | Q429L/L724I/K732E/T1148N | ++ |
| 219/220 | L171P/A362E/V795T/M797Q/K799M | ++ |
| 221/222 | L171P/Q429L/K732E | ++ |
| 223/224 | Q96R/L171P/S643N/M797T/K799R | ++ |
| 225/226 | L171P/T792Y/M797T/K799M | ++ |
| 227/228 | R503S | ++ |
| 229/230 | L171P/K732E | ++ |
| 231/232 | T1148N/F1193L | ++ |
| 233/234 | Q96R/L171P/A362E/Q429L/K732R/ | ++ |
| | V795T/M797Q | |
| 235/236 | K726A | + |
| 237/238 | F59M | + |
| 239/240 | T734R | ++ |
| 241/242 | A362N | + |
| 243/244 | H334G | ++ |
| 245/246 | K732G | ++ |
| 247/248 | V63Q | ++ |
| 249/250 | E351S | ++ |
| 251/252 | L1377D | +++++ |
| 253/254 | V795S | ++ |
| 255/256 | N358K | ++ |
| 257/258 | Q999R | ++ |
| 259/260 | V1250T | ++ |
| 261/262 | E306G | ++ |
| 263/264 | P349G | ++ |
| 265/266 | L1377G | +++ |
| 267/268 | S332F | ++ |
| 269/270 | L1377E | +++++ |
| 271/272 | V257I | ++ |
| 273/274 | L646I | ++ |
| 275/276 | D346R | ++ |
| 277/278 | P435Q | ++ |
| 279/280 | Q96V | ++ |
| 281/282 | L1377R | ++ |
| 283/284 | L1377S | +++ |
| 285/286 | F59G | ++ |
| 287/288 | L171M | ++ |
| 289/290 | T792H | + |
| 291/292 | L1248D | ++ |
| 293/294 | E306S | ++ |
| 295/296 | L706M | ++ |
| 297/298 | L171H | +++ |
| 299/300 | L1377A | ++ |
| 301/302 | L1377V | ++ |
| 303/304 | T734S | ++ |
| 305/306 | T1148R | ++ |
| 307/308 | F57S | ++ |
| 309/310 | F59D | ++ |
| 311/312 | K344E | + |
| 313/314 | A362G | ++ |
| 315/316 | P58K | ++ |

All activities were determined relative to the reference polypeptide of SEQ ID NO: 2. Levels of increased activity are defined as follows: “+” 0.9 to 1.1, “++” >1.1, “+++” >2, “++++” >3.5, “+++++” >5

### Example 5

### FVII Variants Derived from SEQ ID NO: 252

In this Example, experiments conducted to determine FVIII variant activity are described. These variants were tested using the chromogenic assays, as described in Example 3. Results are shown in Tables 5.1 and 5.2 for variants expressed using Expi293F cells.

TABLE 5.1
Activity of FVIII Variants Relative to SEQ ID NO: 252

| | | | Unchallenged |
|---|---|---|---|
| | Amino Acid | Amino Acid | Chromogenic |
| SEQ ID | Differences | Differences | Activity FIOPC |
| NO: | (Relative to | (Relative to | Relative to |
| (nt/aa) | SEQ ID NO: 252) | SEQ ID NO: 2) | SEQ ID NO: 252 |
| 251/252 | | L1377D | + |
| 317/318 | M1380W | L1377D/M1380W | +++ |
| 319/320 | I388A | I388A/L1377D | + |
| 321/322 | F1043E | F1043E/L1377D | + |
| 323/324 | F1043R | F1043R/L1377D | + |
| 325/326 | I610R | I610R/L1377D | + |
| 327/328 | I1274N | I1274N/L1377D | + |
| 329/330 | S1220T | S1220T/L1377D | + |
| 331/332 | I1442T | L1377D/I1442T | + |
| 333/334 | F1193L | F1193L/L1377D | + |
| 335/336 | Q621T | Q621T/L1377D | + |
| 337/338 | L1054C | L1054C/L1377D | + |
| 339/340 | A755Q | A755Q/L1377D | + |
| 341/342 | Y1041G | Y1041G/L1377D | + |
| 343/344 | Y748S | Y748S/L1377D | + |
| 345/346 | Y1049G | Y1049G/L1377D | + |
| 347/348 | L1221V | L1221V/L1377D | + |
| 349/350 | M1051E | M1051E/L1377D | + |
| 351/352 | R612G | R612G/L1377D | + |
| 353/354 | F1037H | F1037H/L1377D | + |
| 355/356 | N616E | N616E/L1377D | + |
| 357/358 | Y1041H | Y1041H/L1377D | + |
| 359/360 | F387S | F387S/L1377D | + |
| 361/362 | S489R | S489R/L1377D | + |
| 363/364 | Q621M | Q621M/L1377D | + |
| 365/366 | S489Q | S489Q/L1377D | + |
| 367/368 | L750G | L750G/L1377D | ++ |
| 369/370 | S1224P | S1224P/L1377D | + |
| 371/372 | A618C | A618C/L1377D | + |
| 373/374 | N616G | N616G/L1377D | + |
| 375/376 | R612T | R612T/L1377D | + |
| 377/378 | I1050Q | I1050Q/L1377D | + |
| 379/380 | T1053A | T1053A/L1377D | + |
| 381/382 | N616W | N616W/L1377D | + |
| 383/384 | I896V | I896V/L1377D | + |
| 385/386 | S489F | S489F/L1377D | + |
| 387/388 | I1050P | I1050P/L1377D | + |
| 389/390 | M897G | M897G/L1377D | + |
| 391/392 | L749T | L749T/L1377D | + |
| 393/394 | I1274V | I1274V/L1377D | + |
| 395/396 | S1220L | S1220L/L1377D | + |
| 397/398 | S260E | S260E/L1377D | + |
| 399/400 | L481V | L481V/L1377D | +++ |
| 401/402 | F613S | F613S/L1377D | + |
| 403/404 | F387G | F387G/L1377D | + |
| 405/406 | Q621R | Q621R/L1377D | + |
| 407/408 | I756R | I756R/L1377D | + |
| 409/410 | V63R | V63R/L1377D | + |

All activities were determined relative to the reference polypeptide of SEQ ID NO: 252. Levels of increased activity are defined as follows: “+” 0.9 to 1.1, “++” >1.1, “+++” >2

TABLE 5.2
Activity of FVIII Variants Relative to SEQ ID NO: 252

| | Amino Acid | Amino Acid | (Expi293F) Unchallenged |
|---|---|---|---|
| SEQ ID | Differences | Differences | Chromogenic Activity |
| NO: | (Relative to | (Relative to | FIOPC Relative to |
| (nt/aa) | SEQ ID NO: 252) | SEQ ID NO: 2) | SEQ ID NO: 252 |
| 251/252 | | L1377D | |
| 411/412 | I265S | I265S/L1377D | + |
| 413/414 | L1221T | L1221T/L1377D | + |
| 415/416 | M1380G | L1377D/M1380G | ++ |
| 417/418 | K752G | K752G/L1377D | + |
| 419/420 | Y748K | Y748K/L1377D | ++ |
| 421/422 | A755L | A755L/L1377D | ++ |
| 423/424 | F1218A | F1218A/L1377D | ++ |
| 425/426 | Y748N | Y748N/L1377D | + |
| 427/428 | T1378E | L1377D/T1378E | ++ |
| 429/430 | L614I | L614I/L1377D | + |
| 431/432 | L749F | L749F/L1377D | ++ |
| 433/434 | F387V | F387V/L1377D | + |
| 435/436 | L750T | L750T/L1377D | + |
| 437/438 | F1193G | F1193G/L1377D | ++ |
| 439/440 | L749V | L749V/L1377D | ++ |
| 441/442 | T1378A | T1378A/L1377D | + |
| 443/444 | L750V | L750V/L1377D | ++ |
| 445/446 | F387T | F387T/L1377D | ++ |
| 447/448 | F387Q | F387Q/L1377D | ++ |
| 449/450 | L750A | L750A/L1377D | ++ |
| 451/452 | F1037N | F1037N/L1377D | ++ |
| 453/454 | H1440A | H1440A/L1377D | ++ |
| 455/456 | M1051S | M1051S/L1377D | +++ |
| 457/458 | L1221G | L1221G/L1377D | ++ |
| 459/460 | T1378D | L1377D/T1378D | ++ |
| 461/462 | H1440S | L1377D/H1440S | +++ |
| 463/464 | F387R | F387R/L1377D | + |
| 465/466 | F1193A | F1193A/L1377D | ++ |
| 467/468 | F387I | F387I/L1377D | ++ |
| 469/470 | F1218M | F1218M/L1377D | ++ |
| 471/472 | I265W | I265W/L1377D | ++ |
| 473/474 | H1440E | L1377D/H1440E | ++ |
| 475/476 | V1439L | L1377D/V1439L | + |
| 477/478 | H1440T | L1377D/H1440T | +++ |
| 479/480 | R612S | R612S/L1377D | + |
| 481/482 | Y1049W | Y1049W/L1377D | + |
| 483/484 | L1221S | L1221S/L1377D | +++ |
| 485/486 | S1220D | S1220D/L1377D | ++ |
| 487/488 | L749G | L749G/L1377D | ++ |
| 489/490 | L749S | L749S/L1377D | + |
| 491/492 | F1218G | F1218G/L1377D | ++ |
| 493/494 | I1205L | I1205L/L1377D | ++ |
| 495/496 | Y1222F | Y1222F/L1377D | ++ |
| 497/498 | F1193E | F1193E/L1377D | ++ |
| 499/500 | I1050S | I1050S/L1377D | ++ |
| 501/502 | S1389W | L1377D/S1389W | + |
| 503/504 | V1439E | L1377D/V1439E | +++ |
| 505/506 | S260T | S260T/L1377D | ++ |
| 507/508 | F1037P | F1037P/L1377D | + |
| 509/510 | M1051K | M1051K/L1377D | +++ |
| 511/512 | I1269F | I1269F/L1377D | + |
| 513/514 | I265R | I265R/L1377D | ++ |
| 515/516 | M1051R | M1051R/L1377D | ++ |
| 517/518 | F1218L | F1218L/L1377D | ++ |
| 519/520 | M897R | M897R/L1377D | ++ |
| 521/522 | I388P | I388P/L1377D | + |
| 523/524 | Y748G | Y748G/L1377D | ++ |
| 525/526 | I756P | I756P/L1377D | ++ |
| 527/528 | Y748F | Y748F/L1377D | + |
| 529/530 | I390A | I390A/L1377D | ++ |
| 531/532 | I610L | I610L/L1377D | + |
| 533/534 | A755G | A755G/L1377D | ++ |
| 535/536 | L749R | L749R/L1377D | ++ |
| 537/538 | Y748V | Y748V/L1377D | + |
| 539/540 | M1380A | L1377D/M1380A | + |
| 541/542 | H1440C | L1377D/H1440C | + |
| 543/544 | H397N/F1218M | H397N/F1218M/L1377D | ++ |
| 545/546 | F1037M | F1037M/L1377D | + |
| 547/548 | F1218P | F1218P/L1377D | + |
| 549/550 | E757K | E757K/L1377D | ++ |
| 551/552 | V1373S | V1373S/L1377D | ++ |
| 553/554 | H1440G | L1377D/H1440G | ++ |
| 555/556 | F1193I | F1193I/L1377D | ++ |
| 557/558 | V1439T | L1377D/V1439T | ++ |
| 559/560 | L750D | L750D/L1377D | ++ |
| 561/562 | I390G | I390G/L1377D | ++ |
| 563/564 | I756G | I756G/L1377D | + |
| 565/566 | S1220E | S1220E/L1377D | ++ |
| 567/568 | T1378C | L1377D/T1378C | + |
| 569/570 | A618G | A618G/L1377D | + |
| 571/572 | D346R | D346R/L1377D | ++ |
| 573/574 | V63Q/L1248D | V63Q/L1248D/L1377D | ++ |
| 575/576 | P349G | P349G/L1377D | ++ |
| 577/578 | E306G/R355C/L1422I | E306G/R355C/L1377D/L1422I | ++ |
| 579/580 | L1422I | L1377D/L1422I | ++ |
| 581/582 | E306G/D346R/I708T | E306G/D346R/I708T/L1377D | ++ |
| 583/584 | T1148N | T1148N/L1377D | +++ |
| 585/586 | L171P/D346R/L1248D | L171P/D346R/L1248D/L1377D | +++ |
| 587/588 | V63Q/M797T/K799R/L1248D/ | V63Q/M797T/K799R/L1248D/ | ++ |
| | L1422I | L1377D/L1422I | |
| 589/590 | L171P/K799R | L171P/K799R/L1377D | ++ |
| 591/592 | A362P | A362P/L1377D | ++ |
| 593/594 | L1248D | L1248D/L1377D | ++ |
| 595/596 | V63Q | V63Q/L1377D | ++ |
| 597/598 | L171P/E306G/D346R/Q999R/ | L171P/E306G/D346R/Q999R/ | +++ |
| | L1248D | L1248D/L1377D | |
| 599/600 | L171P/T1148N | L171P/T1148N/L1377D | +++ |
| 601/602 | T734S/K799R/L1248D/L1422I | T734S/K799R/L1248D/L1377D/ | ++ |
| | | L1422I | |
| 603/604 | V63Q/K732E | V63Q/K732E/L1377D | +++ |
| 605/606 | V63Q/L171P/E306G/D346R/ | V63Q/L171P/E306G/D346R/ | ++ |
| | K732G | K732G/L1377D | |
| 607/608 | L171P | L171P/L1377D | ++ |
| 609/610 | L171P/L1422I | L171P/L1377D/L1422I | ++ |
| 611/612 | V63Q/A362P/K732G/K799R/ | V63Q/A362P/K732G/K799R/ | ++ |
| | L1248D | L1248D/L1377D | |
| 613/614 | Q999R/T1148N/L1422I | Q999R/T1148N/L1377D/L1422I | + |
| 615/616 | D346R/P349G | D346R/P349G/L1377D | +++ |
| 617/618 | E306G/P349G | E306G/P349G/L1377D | ++ |
| 619/620 | V63Q/L171P/T1148N/G1151S | V63Q/L171P/T1148N/L1377D/ | ++ |
| | | G1151S | |
| 621/622 | E306G | E306G/L1377D | + |
| 623/624 | K344E/P349G/L354R/V1250T | K344E/P349G/L354R/V1250T/ | ++ |
| | | L1377D | |
| 625/626 | V63G | V63G/L1377D | + |
| 627/628 | V1250T | V1250T/L1377D | + |
| 629/630 | V63R/E306G/K344E/P349G/ | V63R/E306G/K344E/P349G/ | +++ |
| | L354R/R503S/L1248R | L354R/R503S/L1248R/L1377D | |
| 631/632 | F59D/V63S/S332P/P349G/ | F59D/V63S/S332P/P349G/R503S/ | ++ |
| | R503S/L706M/I708K | L706M/I708K/L1377D | |
| 633/634 | V63S/E306G/K344A/P349G/ | V63S/E306G/K344A/P349G/ | ++ |
| | L354R/L706M/L1248R | L354R/L706M/L1248R/L1377D | |
| 635/636 | L1248R | L1248R/L1377D | ++ |
| 637/638 | F59D/V63G | F59D/V63G/L1377D | ++ |
| 639/640 | S332P/L706M/I708K/L1248R/ | S332P/L706M/I708K/L1248R/ | ++ |
| | V1250T | V1250T/L1377D | |
| 641/642 | R503S | R503S/L1377D | ++ |
| 643/644 | F59D/V63R/P349G/L354R | F59D/V63R/P349G/L354R/L1377D | +++ |
| 645/646 | F59D/P349G/I708K | F59D/P349G/I708K/L1377D | +++ |
| 647/648 | F59D/E306G/K344E/L1248R | F59D/E306G/K344E/L1248R/L1377D | +++ |
| 649/650 | K344E/L1248R/V1250T | K344E/L1248R/V1250T/L1377D | ++ |
| 651/652 | I708K | I708K/L1377D | ++ |
| 653/654 | V63R/K344E/P349G | V63R/K344E/P349G/L1377D | ++ |
| 655/656 | F59D | F59D/L1377D | + |
| 657/658 | V63S/K344A/P349G/L354R | V63S/K344A/P349G/L354R/L1377D | ++ |
| 659/660 | L706M | L706M/L1377D | + |
| 661/662 | F59D/E306G/R770W | F59D/E306G/R770W/L1377D | + |
| 663/664 | V63G/S347N/L354R/L1248R | V63G/S347N/L354R/L1248R/L1377D | ++ |
| 665/666 | L354R/R503S | L354R/R503S/L1377D | +++ |

All activities were determined relative to the reference polypeptide of SEQ ID NO: 252. Levels of increased activity are defined as follows: “+” 0.9 to 1.1, “++” >1.1, “+++” >2

### Example 6

### FVIII Variants Derived from SEQ ID NO: 504

In this Example, experiments conducted to determine FVIII variant activity are described. These variants were tested using the chromogenic and clotting assays, as described in Example 3. Results are shown in Tables 6.1 and 6.2 for variants expressed using Expi293F cells or HepG2 cells.

TABLE 6.1
Activity of FVIII Variants Relative to SEQ ID NO: 504

| | Amino Acid | Amino Acid | (Expi293F) Unchallenged |
|---|---|---|---|
| | Differences | Differences | Chromogenic Activity |
| SEQ ID | (Relative to SEQ | (Relative to | FIOPC Relative to SEQ ID |
| NO: (nt/aa) | ID NO: 504) | SEQ ID NO: 2) | NO: 504 |
| 503/504 | | L1377D/V1439E | + |
| 667/668 | L750D | L750D/L1377D/V1439E | ++ |
| 669/670 | M1051K | M1051K/L1377D/V1439E | ++ |
| 671/672 | M1051S | M1051S/L1377D/V1439E | ++ |
| 673/674 | S260T | S260T/L1377D/V1439E | ++ |
| 675/676 | I756P | I756P/L1377D/V1439E | + |
| 677/678 | F1193A | F1193A/L1377D/V1439E | ++ |
| 679/680 | S332P | S332P/L1377D/V1439E | + |
| 681/682 | M897R | M897R/L1377D/V1439E | ++ |
| 683/684 | F387Q | F387Q/L1377D/V1439E | + |
| 685/686 | F1037N | F1037N/L1377D/V1439E | + |
| 687/688 | F387R | F387R/L1377D/V1439E | ++ |
| 689/690 | E306G/L749F | E306G/L749F/L1377D/V1439E | ++ |
| 691/692 | I1270V | I1270V/L1377D/V1439E | + |
| 693/694 | S489E | S489E/L1377D/V1439E | ++ |
| 695/696 | Q487N | Q487N/L1377D/V1439E | ++ |
| 697/698 | A488G | A488G/L1377D/V1439E | ++ |
| 699/700 | Q903E | Q903E/L1377D/V1439E | + |

All activities were determined relative to the reference polypeptide of SEQ ID NO: 504. Levels of increased activity are defined as follows: “+” 0.9 to 1.1, “++” >1.1

TABLE 6.2
Activity of FVIII Variants Relative to SEQ ID NO: 504

| | | | (Expi293F) | (HepG2) | (Expi293F) |
|---|---|---|---|---|---|
| | | | Unchallenged | Unchallenged | Unchallenged |
| | | | Chromogenic | Chromogenic | Clotting |
| | Amino Acid | Amino Acid | Activity | Activity | Activity |
| SEQ ID | Differences | Differences | FIOPC | FIOPC | FIOPC |
| NO: | (Relative to | (Relative to | Relative to | Relative to | Relative to |
| (nt/aa) | SEQ ID NO: 504) | SEQ ID NO: 2) | SEQ ID NO:504 | SEQ ID NO: 504 | SEQ ID NO: 504 |
| 503/504 | | L1377D/V1439E | + | + | + |
| 701/702 | L171P/L1248D/H1440T | L171P/L1248D/ | +++ | +++ | ++ |
| | | L1377D/V1439E/H1440T | | | |
| 703/704 | S260T/T1148N/T1378D | S260T/T1148N/L1377D/ | +++ | + | + |
| | | T1378D/V1439E | | | |
| 705/706 | L171P/S260T/F387I/ | L171P/S260T/F387I/ | +++ | +++ | +++ |
| | L750D/L1248D | L750D/L1248D/L1377D/ | | | |
| | | V1439E | | | |
| 707/708 | L171P/S260T | L171P/S260T/L1377D/ | +++ | +++ | ++ |
| | | V1439E | | | |
| 709/710 | L171P/S260T/F387I/ | L171P/S260T/F387I/ | +++ | +++ | +++ |
| | L750D/M1051S/T1148N | L750D/M1051S/T1148N/ | | | |
| | | L1377D/V1439E | | | |
| 711/712 | L171P | L171P/L1377D/V1439E | +++ | +++ | ++ |
| 713/714 | L750D/M1051S/T1148N | L750D/M1051S/T1148N/ | +++ | ++ | ++ |
| | | L1377D/V1439E | | | |
| 715/716 | L171P/S260T/I756P | L171P/S260T/I756P/ | +++ | +++ | ++ |
| | | L1377D/V1439E | | | |
| 717/718 | L171P/L1248D | L171P/L1248D/L1377D/ | +++ | +++ | ++ |
| | | V1439E | | | |
| 719/720 | L171P/T1148N | L171P/T1148N/L1377D/ | ++ | +++ | ++ |
| | | V1439E | | | |
| 721/722 | F387I | F387I/L1377D/V1439E | ++ | + | + |
| 723/724 | F59D | F59D/L1377D/V1439E | ++ | + | ++ |
| 725/726 | H1440S | L1377D/V1439E/H1440S | ++ | + | + |
| 727/728 | S332P/L750D/L1248D | S332P/L750D/L1248D/ | +++ | ++ | +++ |
| | | L1377D/V1439E | | | |
| 729/730 | I756P/L1248D | I756P/L1248D/L1377D/ | ++ | + | ++ |
| | | V1439E | | | |
| 731/732 | L1248D | L1248D/L1377D/V1439E | ++ | | ++ |
| 733/734 | L171P/F387I/L1248D/ | L171P/F387I/L1248D/ | +++ | ++ | ++ |
| | H1440S | L1377D/H1440S/V1439E | | | |
| 735/736 | L750D/H1440S | L750D/L1377D/V1439E/ | ++ | ++ | ++ |
| | | H1440S | | | |
| 737/738 | F59D/L171P/L750D | F59D/L171P/L750D/ | +++ | +++ | +++ |
| | | L1377D/V1439E | | | |
| 739/740 | L171P/F1193A/L1248D | L171P/F1193A/L1248D/ | +++ | +++ | ++ |
| | | L1377D/V1439E | | | |
| 741/742 | L750D/T1148N/ | L750D/T1148N/F1193A/ | +++ | ++ | ++ |
| | F1193A/H1440S | L1377D/V1439E/H1440S | | | |
| 743/744 | T1148N | T1148N/L1377D/V1439E | ++ | ++ | ++ |
| 745/746 | L171P/F387I/M1051K | L171P/F387I/M1051K/ | ++ | +++ | ++ |
| | | L1377D/V1439E | | | |
| 747/748 | Y748G | Y748G/L1377D/V1439E | ++ | + | + |
| 749/750 | E306G | E306G/L1377D/V1439E | | | ++ |
| 751/752 | E306G/F387R/I390A/ | E306G/F387R/I390A/ | +++ | +++ | ++ |
| | L749F/S1220D | L749F/S1220D/L1377D/ | | | |
| | | V1439E | | | |
| 753/754 | I390A | I390A/L1377D/V1439E | ++ | +++ | ++ |
| 755/756 | E306G/I390A/F1037N | E306G/I390A/F1037N/ | ++ | ++ | ++ |
| | | L1377D/V1439E | | | |
| 757/758 | E306G/R612S/L749F | E306G/R612S/L749F/ | ++ | + | ++ |
| | | L1377D/V1439E | | | |
| 759/760 | F387V/I390A/L749F | F387V/I390A/L749F/ | +++ | ++ | ++ |
| | | L1377D/V1439E | | | |
| 761/762 | V63S/I390A/R503S/ | V63S/I390A/R503S/ | ++ | ++ | ++ |
| | L749F | L749F/L1377D/V1439E | | | |
| 763/764 | F387R/I390A/L749R | F387R/I390A/L749R/ | ++ | ++ | ++ |
| | | L1377D/V1439E | | | |
| 765/766 | S1220D | S1220D/L1377D/V1439E | ++ | | |
| 767/768 | V63S/E306G | V63S/E306G/L1377D/ | ++ | ++ | ++ |
| | | V1439E | | | |
| 769/770 | L749F | L749F/L1377D/V1439E | | ++ | ++ |
| 771/772 | E306G/I390A/L749F/ | E306G/I390A/L749F/ | ++ | ++ | ++ |
| | F1037N | F1037N/L1377D/V1439E | | | |
| 773/774 | F387V | F387V/L1377D/V1439E | ++ | ++ | + |
| 775/776 | E306G/L749F/S1220D | E306G/L749F/S1220D/ | ++ | ++ | + |
| | | L1377D/V1439E | | | |
| 777/778 | E306G/F387V/I390A/ | E306G/F387V/I390A/ | ++ | ++ | ++ |
| | R612S/L749F/ | R612S/L749F/F1037N/ | | | |
| | F1037N | L1377D/V1439E | | | |
| 779/780 | F328S | F328S/L1377D/V1439E | ++ | ++ | + |

All activities were determined relative to the reference polypeptide of SEQ ID NO: 504. Level of increased activity are defined as follows: “+” 0.9 to 1.1, “++” >1.1, “+++” >2

### Example 7

### FVIII Variants Derived from SEQ ID NO: 706

In this Example, experiments conducted to determine FVIII variant activity are described. These variants were tested using the chromogenic and clotting assays, as described in Example 3. Results are shown in Tables 7.1 and 7.2 for variants expressed using Expi293F cells or HepG2 cells.

TABLE 7.1
Activity of FVIII Variants Relative to SEQ ID NO: 706

| | | | (Expi293F) |
|---|---|---|---|
| | Amino Acid | | Unchallenged |
| | Differences | Amino Acid | Chromogenic |
| | (Relative to | Differences | Activity FIOPC |
| SEQ ID | SEQ ID NO: | (Relative to | Relative to |
| NO: (nt/aa) | 706) | SEQ ID NO: 2) | SEQ ID NO: 706 |
| 705/706 | | L171P/S260T/F387I/L750D/L1248D/ | + |
| | | L1377D/V1439E | |
| 781/782 | M1051K | L171P/S260T/F387I/L750D/M1051K/ | ++ |
| | | L1248D/L1377D/V1439E | |
| 783/784 | L1221G | L171P/S260T/F387I/L750D/L1221G/ | + |
| | | L1248D/L1377D/V1439E | |
| 785/786 | F1218G | L171P/S260T/F387I/L750D/F1218G/ | + |
| | | L1248D/L1377D/V1439E | |
| 787/788 | I387R | L171P/S260T/F387R/L750D/L1248D/ | + |
| | | L1377D/V1439E | |
| 789/790 | M1051R | L171P/S260T/F387I/L750D/M1051R/ | ++ |
| | | L1248D/L1377D/V1439E | |
| 791/792 | L749F/ | L171P/S260T/F387I/L749F/L750D/ | ++ |
| | F1218G | F1218G/L1248D/L1377D/V1439E | |
| 793/794 | I387T | L171P/S260T/F387T/L750D/L1248D/ | + |
| | | L1377D/V1439E | |
| 795/796 | S332P | L171P/S260T/S332P/F387I/L750D/ | + |
| | | L1248D/L1377D/V1439E | |
| 797/798 | M897K | L171P/S260T/F387I/L750D/M897K/ | + |
| | | L1248D/L1377D/V1439E | |
| 799/800 | T696E | L171P/S260T/F387I/T696E/L750D/ | + |
| | | L1248D/L1377D/V1439E | |
| 801/802 | F816W | L171P/S260T/F387I/L750D/F816W/ | + |
| | | L1248D/L1377D/V1439E | |
| 803/804 | F824W | L171P/S260T/F387I/L750D/F824W/ | + |
| | | L1248D/L1377D/V1439E | |
| 805/806 | A826G | L171P/S260T/F387I/L750D/A826G/ | + |
| | | L1248D/L1377D/V1439E | |
| 807/808 | V393W | L171P/S260T/F387I/V393W/L750D/ | + |
| | | L1248D/L1377D/V1439E | |
| 809/810 | I825L | L171P/S260T/F387I/L750D/I825L/ | + |
| | | L1248D/L1377D/V1439E | |
| 811/812 | M897R | L171P/S260T/F387I/L750D/M897R/ | + |
| | | L1248D/L1377D/V1439E | |
| 813/814 | F816V | L171P/S260T/F387I/L750D/F816V/ | ++ |
| | | L1248D/L1377D/V1439E | |
| 815/816 | F698W | L171P/S260T/F387I/F698W/L750D/ | ++ |
| | | L1248D/L1377D/V1439E | |
| 817/818 | V393Q | L171P/S260T/F387I/V393Q/L750D/ | + |
| | | L1248D/L1377D/V1439E | |

All activities were determined relative to the reference polypeptide of SEQ ID NO: 706. Levels of increased activity are defined as follows: “+” 0.9 to 1.1, “++” >1.1

TABLE 7.2
Activity of FVIII Variants Relative to SEQ ID NO: 706

| | | | (Expi293F) | (HepG2) | (Expi293F) |
|---|---|---|---|---|---|
| | | | Unchallenged | Unchallenged | Unchallenged |
| | | | Chromogenic | Chromogenic | Clotting |
| | Amino Acid | Amino Acid | Activity | Activity | Activity |
| SEQ ID | Differences | Differences | FIOPC | FIOPC | FIOPC |
| NO: | (Relative to | (Relative to | Relative to | Relative to | Relative to |
| (nt/aa) | SEQ ID NO: 706) | SEQ ID NO: 2) | SEQ ID NO: 706 | SEQ ID NO: 706 | SEQ ID NO: 706 |
| 705/706 | | L171P/S260T/F387I/L750D/ | + | + | + |
| | | L1248D/L1377D/V1439E | | | |
| 819/820 | S332P/I387R/ | L171P/S260T/S332P/F387R/ | +++ | +++ | +++ |
| | I390A/T1148N | I390A/L750D/T1148N/L1248D/ | | | |
| | | L1377D/V1439E | | | |
| 821/822 | F328S/I387R/ | L171P/S260T/F328S/F387R/ | + | + | ++ |
| | L749F | L749F/L750D/L1248D/L1377D/ | | | |
| | | V1439E | | | |
| 823/824 | L749F | L171P/S260T/F387I/L749F/ | + | + | + |
| | | L750D/L1248D/L1377D/V1439E | | | |
| 825/826 | E306G/F328S/ | L171P/S260T/E306G/F328S/ | +++ | +++ | + |
| | S332P/I390A/ | S332P/F387I/I390A/L750D/ | | | |
| | M1051R/F1193A/ | M1051R/F1193A/F1218G/L1248D/ | | | |
| | F1218G | L1377D/V1439E | | | |
| 827/828 | V63Q/E306G/ | V63Q/L171P/S260T/E306G/ | +++ | +++ | +++ |
| | S332P/M1051R/ | S332P/F387I/L750D/M1051R/ | | | |
| | T1148N | T1148N/L1248D/L1377D/V1439E | | | |
| 829/830 | V63Q/E306G/ | V63Q/L171P/S260T/E306G/ | ++ | +++ | ++ |
| | S332P/I387T/ | S332P/F387T/L750D/M1051K/ | | | |
| | M1051K | L1248D/L1377D/V1439E | | | |
| 831/832 | I387T/I390A/ | L171P/S260T/F387T/I390A/ | ++ | +++ | ++ |
| | M1051R | L750D/M1051R/L1248D/L1377D/ | | | |
| | | V1439E | | | |
| 833/834 | D186A/E306G/ | L171P/D186A/S260T/E306G/ | +++ | ++ | +++ |
| | F328S/S332P/ | F328S/S332P/F387I/I390A/ | | | |
| | I390A/L749F/ | L749F/L750D/M1051K/T1148N/ | | | |
| | M1051K/T1148N/ | F1193A/L1248D/L1377D/V1439E | | | |
| | F1193A | | | | |
| 835/836 | I387T/I390A/ | L171P/S260T/F387T/I390A/ | ++ | ++ | ++ |
| | I756P/M1051R | L750D/I756P/M1051R/L1248D/ | | | |
| | | L1377D/V1439E | | | |
| 837/838 | E306G/I390A/ | L171P/S260T/E306G/F387I/ | +++ | ++ | +++ |
| | M1051R | I390A//L750D/M1051R/ | | | |
| | | L1248D/L1377D/V1439E | | | |
| 839/840 | E306G/S332P/ | L171P/S260T/E306G/S332P/ | +++ | +++ | +++ |
| | I390A/L749F | F387I/I390A/L749F/L750D/ | | | |
| | | L1248D/L1377D/V1439E | | | |
| 841/842 | E306G/I387R/ | L171P/S260T/E306G/F387R/ | ++ | ++ | +++ |
| | I390A/T1148N | I390A/L750D/T1148N/L1248D/ | | | |
| | | L1377D/V1439E | | | |
| 843/844 | E306G/F328S/ | L171P/S260T/E306G/F328S/ | +++ | +++ | +++ |
| | S332P/I387T/ | S332P/F387T/I390A/L749F/ | | | |
| | I390A/L749F/ | L750D/M1051R/T1148N/F1193A/ | | | |
| | M1051R/T1148N/ | L1248D/L1377D/V1439E | | | |
| | F1193A | | | | |
| 845/846 | S332P/I387R/ | L171P/S260T/S332P/F387R/ | +++ | ++ | +++ |
| | I390A/L749F/ | I390A/L749F/L750D/T1148N/ | | | |
| | T1148N | L1248D/L1377D/V1439E | | | |
| 847/848 | I387R/I390A/ | L171P/S260T/F387R/I390A/ | ++ | ++ | ++ |
| | L749F | L749F/L750D/L1248D/L1377D/ | | | |
| | | V1439E | | | |
| 849/850 | E306G/I387T/ | L171P/S260T/E306G/F387T/ | ++ | ++ | ++ |
| | L749F/M1051K/ | L749F/L750D/M1051K/T1148N/ | | | |
| | T1148N | L1248D/L1377D/V1439E | | | |
| 851/852 | I387R/L749F | L171P/S260T/F387R/L749F/ | ++ | ++ | ++ |
| | | L750D/L1248D/L1377D/V1439E | | | |
| 853/854 | E306G/Q945K/ | L171P/S260T/E306G/F387I/ | ++ | + | + |
| | M1051R/F1193A/ | L750D/Q945K/M1051R/F1193A/ | | | |
| | L1221G | L1221G/L1248D/L1377D/V1439E | | | |
| 855/856 | E306G/I390A/ | L171P/S260T/E306G/F387I/ | ++ | + | ++ |
| | L749F/M1051K | I390A/L749F/L750D/M1051K/ | | | |
| | | L1248D/L1377D/V1439E | | | |
| 857/858 | V63Q/E306G/ | V63Q/L171P/S260T/E306G/ | ++ | ++ | +++ |
| | S332P/I390A | S332P/F387I/I390A/L750D/ | | | |
| | | L1248D/L1377D/V1439E | | | |
| 859/860 | E306G/I387T/ | L171P/S260T/E306G/F387T/ | +++ | ++ | + |
| | I390A/L749F/ | I390A/L749F/L750D/T1148N/ | | | |
| | T1148N/F1193A/ | F1193A/L1221G/L1248D/L1377D/ | | | |
| | L1221G | V1439E | | | |
| 861/862 | E306G/S332P/ | L171P/S260T/E306G/S332P/ | +++ | +++ | ++ |
| | I387R/I390A/ | F387R//I390A/L749F/ | | | |
| | L749F/F1218M | L750D/F1218M/L1248D/L1377D/ | | | |
| | | V1439E | | | |
| 863/864 | V63Q/E306G/ | V63Q/L171P/S260T/E306G/ | +++ | +++ | + |
| | S332P/I390A/ | S332P/F387I/I390A/L750D/ | | | |
| | M1051R/T1148N/ | M1051R/T1148N/F1193A/F1218G/ | | | |
| | F1193A/F1218G | L1248D/L1377D/V1439E | | | |
| 865/866 | G924I | L171P/S260T/F387I/L750D/ | + | ++ | + |
| | | G924I/L1248D/L1377D/V1439E | | | |
| 867/868 | A755S | L171P/S260T/F387I/L750D/ | + | + | + |
| | | A755S/L1248D/L1377D/V1439E | | | |
| 869/870 | K752F | L171P/S260T/F387I/L750D/ | + | + | ++ |
| | | K752F/L1248D/L1377D/V1439E | | | |
| 871/872 | S751Y | L171P/S260T/F387I/L750D/ | ++ | ++ | ++ |
| | | S751Y/L1248D/L1377D/V1439E | | | |
| 873/874 | Q923M | L171P/S260T/F387I/L750D/ | + | + | + |
| | | Q923M/L1248D/L1377D/V1439E | | | |
| 875/876 | M797Y | L171P/S260T/F387I/L750D/ | + | + | + |
| | | M797Y/L1248D/L1377D/V1439E | | | |
| 877/878 | N754W | L171P/S260T/F387I/L750D/ | + | + | + |
| | | N754W/L1248D/L1377D/V1439E | | | |
| 879/880 | I793G | L171P/S260T/F387I/L750D/ | + | + | + |
| | | 1793G/L1248D/L1377D/V1439E | | | |
| 881/882 | N753D | L171P/S260T/F387I/L750D/ | ++ | + | +++ |
| | | N753D/L1248D/L1377D/V1439E | | | |
| 883/884 | R922W | L171P/S260T/F387I/L750D/ | + | + | + |
| | | R922W/L1248D/L1377D/V1439E | | | |
| 885/886 | I896L | L171P/S260T/F387I/L750D/ | + | + | + |
| | | 1896L/L1248D/L1377D/V1439E | | | |
| 887/888 | K938R | L171P/S260T/F387I/L750D/ | + | + | + |
| | | K938R/L1248D/L1377D/V1439E | | | |
| 889/890 | I775G | L171P/S260T/F387I/L750D/ | ++ | ++ | ++ |
| | | I775G/L1248D/L1377D/V1439E | | | |
| 891/892 | S751E | L171P/S260T/F387I/L750D/ | + | + | + |
| | | S751E/L1248D/L1377D/V1439E | | | |
| 893/894 | K732W | L171P/S260T/F387I/L750D/ | + | + | ++ |
| | | K732W/L1248D/L1377D/V1439E | | | |
| 895/896 | K752G | L171P/S260T/F387I/L750D/ | + | + | ++ |
| | | K752G/L1248D/L1377D/V1439E | | | |
| 897/898 | N810V | L171P/S260T/F387I/L750D/ | + | + | + |
| | | N810V/L1248D/L1377D/V1439E | | | |
| 899/900 | K938V | L171P/S260T/F387I/L750D/ | + | + | + |
| | | K938V/L1248D/L1377D/V1439E | | | |
| 901/902 | A747D | L171P/S260T/F387I/L750D/ | ++ | + | ++ |
| | | A747D/L1248D/L1377D/V1439E | | | |
| 903/904 | A755G | L171P/S260T/F387I/L750D/ | + | + | + |
| | | A755G/L1248D/L1377D/V1439E | | | |
| 905/906 | V393G/A407V | L171P/S260T/F387I/V393G/ | ++ | ++ | + |
| | | A407V/L750D/L1248D/L1377D/ | | | |
| | | V1439E | | | |
| 907/908 | M797W | L171P/S260T/F387I/L750D/ | + | + | ++ |
| | | M797W/L1248D/L1377D/V1439E | | | |
| 909/910 | K819V | L171P/S260T/F387I/L750D/ | + | + | + |
| | | K819V/L1248D/L1377D/V1439E | | | |
| 911/912 | V393G | L171P/S260T/F387I/V393G/ | ++ | +++ | + |
| | | L750D/L1248D/L1377D/V1439E | | | |
| 913/914 | I787W | L171P/S260T/F387I/L750D/ | + | ++ | ++ |
| | | I787W/L1248D/L1377D/V1439E | | | |
| 915/916 | V393R | L171P/S260T/F387I/V393R/ | ++ | +++ | + |
| | | L750D/L1248D/L1377D/V1439E | | | |
| 917/918 | F802V | L171P/S260T/F387I/L750D/ | + | ++ | + |
| | | F802V/L1248D/L1377D/V1439E | | | |
| 919/920 | I775E | L171P/S260T/F387I/L750D/ | ++ | + | + |
| | | I775E/L1248D/L1377D/V1439E | | | |
| 921/922 | G924P | L171P/S260T/F387I/L750D/ | + | + | + |
| | | G924P/L1248D/L1377D/V1439E | | | |
| 923/924 | K732G | L171P/S260T/F387I/K732G/ | + | + | + |
| | | L750D/L1248D/L1377D/V1439E | | | |
| 925/926 | Q811F | L171P/S260T/F387I/L750D/ | + | + | ++ |
| | | Q811F/L1248D/L1377D/V1439E | | | |
| 927/928 | F802S | L171P/S260T/F387I/L750D/ | + | ++ | + |
| | | F802S/L1248D/L1377D/V1439E | | | |
| 929/930 | K752L | L171P/S260T/F387I/L750D/ | + | + | +++ |
| | | K752L/L1248D/L1377D/V1439E | | | |
| 931/932 | S392A | L171P/S260T/F387I/S392A/ | + | + | + |
| | | L750D/L1248D/L1377D/V1439E | | | |
| 933/934 | H397G | L171P/S260T/F387I/H397G/ | + | ++ | + |
| | | L750D/L1248D/L1377D/V1439E | | | |

All activities were determined relative to the reference polypeptide of SEQ ID NO: 706. Levels of increased activity are defined as follows: “+” 0.9 to 1.1, “++” >1.1, “+++” >2

### Example 8

### FVIII Variants Derived from SEQ ID NO: 840

In this Example, experiments conducted to determine FVIII variant activity by assaying the protein activity after a challenge is described. These variants were tested for FVIII activity after no incubation and after incubation in Hemophilia A patient plasma (George King Bio-Medical, Inc.), as described in Example 3. Variants were tested using the chromogenic and clotting assays, as described in Example 3. Results are shown in Tables 8.1 and 8.2 for variants expressed using Expi293F cells or HepG2 cells.

TABLE 8.1
Activity of FVIII Variants Relative to SEQ ID NO: 840

| | | | (Expi293F) | (Expi293F) |
|---|---|---|---|---|
| | | | Unchallenged | Activity after |
| | | | Chromogenic | Plasma |
| | Amino Acid | | Activity | Challenge |
| | Differences | Amino Acid | FIOPC | FIOPC |
| SEQ | (Relative to | Differences | Relative to | Relative to |
| ID NO: | SEQ ID NO: | (Relative to | SEQ ID NO: | SEQ ID NO: |
| (nt/aa) | 840) | SEQ ID NO: 2) | 840 | 840 |
| 839/840 | | L171P/S260T/E306G/S332P/F387I/I390A/ | + | + |
| | | L749F/L750D/L1248D/L1377D/V1439E | | |
| 935/936 | V620N | L171P/S260T/E306G/S332P/F387I/I390A/ | + | + |
| | | V620N/L749F/L750D/L1248D/L1377D/V1439E | | |
| 937/938 | P491E | L171P/S260T/E306G/S332P/F387I/I390A/ | ++ | + |
| | | P491E/L749F/L750D/L1248D/L1377D/V1439E | | |
| 939/940 | M1229D | L171P/S260T/E306G/S332P/F387I/I390A/ | + | + |
| | | L749F/L750D/M1229D/L1248D/L1377D/V1439E | | |
| 941/942 | P615N | L171P/S260T/E306G/S332P/F387I/I390A/ | ++ | |
| | | P615N/L749F/L750D/L1248D/L1377D/V1439E | | |
| 943/944 | I1283S | L171P/S260T/E306G/S332P/F387I/I390A/ | ++ | |
| | | L749F/L750D/L1248D/I1283S/L1377D/V1439E | | |
| 945/946 | H822S | L171P/S260T/E306G/S332P/F387I/I390A/ | ++ | |
| | | L749F/L750D/H822S/L1248D/L1377D/V1439E | | |
| 947/948 | F328G | L171P/S260T/E306G/F328G/S332P/F387I/ | ++ | + |
| | | I390A/L749F/L750D/L1248D/L1377D/V1439E | | |
| 949/950 | H212L | L171P/H212L/S260T/E306G/S332P/F387I/ | +++ | ++ |
| | | I390A/L749F/L750D/L1248D/L1377D/V1439E | | |
| 951/952 | K818W | L171P/S260T/E306G/S332P/F387I/I390A/ | ++ | + |
| | | L749F/L750D/K818W/L1248D/L1377D/V1439E | | |
| 953/954 | I1283L | L171P/S260T/E306G/S332P/F387I/I390A/ | + | ++ |
| | | L749F/L750D/L1248D/I1283L/L1377D/V1439E | | |
| 955/956 | F555G/ | L171P/S260T/E306G/S332P/F387I/I390A/ | ++ | + |
| | E684G | F555G/E684G/L749F/L750D/L1248D/L1377D/ | | |
| | | V1439E | | |
| 957/958 | V828K | L171P/S260T/E306G/S332P/F387I/I390A/ | ++ | ++ |
| | | L749F/L750D/V828K/L1248D/L1377D/V1439E | | |
| 959/960 | R550G | L171P/S260T/E306G/S332P/F387I/I390A/ | + | + |
| | | R550G/L749F/L750D/L1248D/L1377D/V1439E | | |
| 961/962 | R830M | L171P/S260T/E306G/S332P/F387I/I390A/ | ++ | + |
| | | L749F/L750D/R830M/L1248D/L1377D/V1439E | | |
| 963/964 | S839A | L171P/S260T/E306G/S332P/F387I/I390A/ | + | + |
| | | L749F/L750D/S839A/L1248D/L1377D/V1439E | | |
| 965/966 | H822T | L171P/S260T/E306G/S332P/F387I/I390A/ | + | + |
| | | L749F/L750D/H822T/L1248D/L1377D/V1439E | | |
| 967/968 | L13Y | L13Y/L171P/S260T/E306G/S332P/F387I/ | ++ | ++ |
| | | I390A/L749F/L750D/L1248D/L1377D/V1439E | | |
| 969/970 | L14G | L14G/L171P/S260T/E306G/S332P/F387I/ | + | + |
| | | I390A/L749F/L750D/L1248D/L1377D/V1439E | | |
| 971/972 | C17V | C17V/L171P/S260T/E306G/S332P/F387I/ | ++ | |
| | | I390A/L749F/L750D/L1248D/L1377D/V1439E | | |
| 973/974 | M836V | L171P/S260T/E306G/S332P/F387I/I390A/ | + | ++ |
| | | L749F/L750D/M836V/L1248D/L1377D/V1439E | | |
| 975/976 | S839P | L171P/S260T/E306G/S332P/F387I/I390A/ | + | + |
| | | L749F/L750D/S839P/L1248D/L1377D/V1439E | | |
| 977/978 | K1210W | L171P/S260T/E306G/S332P/F387I/I390A/ | + | + |
| | | L749F/L750D/K1210W/L1248D/L1377D/V1439E | | |
| 979/980 | Y1281R | L171P/S260T/E306G/S332P/F387I/I390A/ | ++ | |
| | | L749F/L750D/L1248D/Y1281R/L1377D/V1439E | | |
| 981/982 | V64D | V64D/L171P/S260T/E306G/S332P/F387I/ | ++ | |
| | | I390A/L749F/L750D/L1248D/L1377D/V1439E | | |
| 983/984 | H212G | L171P/H212G/S260T/E306G/S332P/F387I/ | ++ | ++ |
| | | I390A/L749F/L750D/L1248D/L1377D/V1439E | | |
| 985/986 | V682R | L171P/S260T/E306G/S332P/F387I/I390A/ | ++ | |
| | | V682R/L749F/L750D/L1248D/L1377D/V1439E | | |
| 987/988 | L14M | L14M/L171P/S260T/E306G/S332P/F387I/ | ++ | |
| | | I390A/L749F/L750D/L1248D/L1377D/V1439E | | |
| 989/990 | L318C | L171P/S260T/E306G/L318C/S332P/F387I/ | | ++ |
| | | I390A/L749F/L750D/L1248D/L1377D/V1439E | | |
| 991/992 | C17S | C17S/L171P/S260T/E306G/S332P/F387I/ | ++ | ++ |
| | | I390A/L749F/L750D/L1248D/L1377D/V1439E | | |
| 993/994 | A20L | A20L/L171P/S260T/E306G/S332P/F387I/ | ++ | ++ |
| | | I390A/L749F/L750D/L1248D/L1377D/V1439E | | |
| 995/996 | K818G | L171P/S260T/E306G/S332P/F387I/I390A/ | + | + |
| | | L749F/L750D/K818G/L1248D/L1377D/V1439E | | |
| 997/998 | M836G | L171P/S260T/E306G/S332P/F387I/I390A/ | + | + |
| | | L749F/L750D/M836G/L1248D/L1377D/V1439E | | |
| 999/1000 | T21M | T21M/L171P/S260T/E306G/S332P/F387I/ | ++ | ++ |
| | | I390A/L749F/L750D/L1248D/L1377D/V1439E | | |
| 1001/1002 | M836R | L171P/S260T/E306G/S332P/F387I/I390A/ | + | ++ |
| | | L749F/L750D/M836R/L1248D/L1377D/V1439E | | |
| 1003/1004 | F325Y | L171P/S260T/E306G/F325Y/S332P/F387I/ | ++ | ++ |
| | | I390A/L749F/L750D/L1248D/L1377D/V1439E | | |
| 1005/1006 | T533V | L171P/S260T/E306G/S332P/F387I/I390A/ | ++ | |
| | | T533V/L749F/L750D/L1248D/L1377D/V1439E | | |
| 1007/1008 | S19G | S19G/L171P/S260T/E306G/S332P/F387I/ | ++ | |
| | | I390A/L749F/L750D/L1248D/L1377D/V1439E | | |
| 1009/1010 | L14V | L14V/L171P/S260T/E306G/S332P/F387I/ | ++ | |
| | | I390A/L749F/L750D/L1248D/L1377D/V1439E | | |
| 1011/1012 | Q817D | L171P/S260T/E306G/S332P/F387I/I390A/ | + | + |
| | | L749F/L750D/Q817D/L1248D/L1377D/V1439E | | |
| 1013/1014 | L831W | L171P/S260T/E306G/S332P/F387I/I390A/ | + | + |
| | | L749F/L750D/L831W/L1248D/L1377D/V1439E | | |
| 1015/1016 | K225R | L171P/K225R/S260T/E306G/S332P/F387I/ | ++ | ++ |
| | | I390A/L749F/L750D/L1248D/L1377D/V1439E | | |
| 1017/1018 | H1156R | L171P/S260T/E306G/S332P/F387I/I390A/ | + | ++ |
| | | L749F/L750D/H1156R/L1248D/L1377D/V1439E | | |
| 1019/1020 | R830V | L171P/S260T/E306G/S332P/F387I/I390A/ | ++ | ++ |
| | | L749F/L750D/R830V/L1248D/L1377D/V1439E | | |
| 1021/1022 | V393G/ | L171P/S260T/E306G/S332P/F387I/I390A/ | ++ | ++ |
| | A747D | V393G/A747D/L749F/L750D/L1248D/L1377D/ | | |
| | | V1439E | | |
| 1023/1024 | N753D | L171P/S260T/E306G/S332P/F387I/I390A/ | ++ | |
| | | L749F/L750D/N753D/L1248D/L1377D/V1439E | | |
| 1025/1026 | V393G/ | L171P/S260T/E306G/S332P/F387I/I390A/ | ++ | ++ |
| | K732W/ | V393G/K732W/L749F/L750D/S751Y/L1248D/ | | |
| | S751Y | L1377D/V1439E | | |
| 1027/1028 | V63Q/ | V63Q/L171P/S260T/E306G/S332P/F387I/ | ++ | + |
| | M797W | I390A/L749F/L750D/M797W/L1248D/L1377D/ | | |
| | | V1439E | | |

All activities were determined relative to the reference polypeptide of SEQ ID NO: 840. Levels of increased activity are defined as follows: “+” 0.9 to 1.1, “++” >1.1, “+++” >2

TABLE 8.2
Activity of FVIII Variants Relative to SEQ ID NO: 840

| | | | | (Expi293F) | | |
|---|---|---|---|---|---|---|
| | | | (Expi293F) | Chromogenic | (HepG2) | (Expi293F) |
| | | | Unchallenged | Activity | Unchallenged | Unchallenged |
| | | | Chromogenic | after Plasma | Chromogenic | Clotting |
| | | | Activity | Challenge | Activity | Activity |
| | Amino Acid | Amino Acid | FIOPC | FIOPC | FIOPC | FIOPC |
| SEQ ID | Differences | Differences | Relative to | Relative to | Relative to | Relative to |
| NO: | (Relative to | (Relative to | SEQ ID NO: | SEQ ID NO: | SEQ ID NO: | SEQ ID NO: |
| (nt/aa) | SEQ ID NO: 840) | SEQ ID NO: 2) | 840 | 840 | 840 | 840 |
| 839/840 | | L171P/S260T/E306G/S332P/F387I/I390A/L749F/L750D/ | + | + | + | + |
| | | L1248D/L1377D/V1439E | | | | |
| 1029/1030 | L622D | L171P/S260T/E306G/S332P/F387I/I390A/L622D/L749F/ | ++ | + | + | + |
| | | L750D/L1248D/L1377D/V1439E | | | | |
| 1031/1032 | P617E | L171P/S260T/E306G/S332P/F387I/I390A/P617E/L749F/ | ++ | ++ | + | ++ |
| | | L750D/L1248D/L1377D/V1439E | | | | |
| 1033/1034 | P617N | L171P/S260T/E306G/S332P/F387I/I390A/P617N/L749F/ | ++ | | + | + |
| | | L750D/L1248D/L1377D/V1439E | | | | |
| 1035/1036 | P907D | L171P/S260T/E306G/S332P/F387I/I390A/L749F/L750D/ | ++ | + | | + |
| | | P907D/L1248D/L1377D/V1439E | | | | |
| 1037/1038 | N1040E | L171P/S260T/E306G/S332P/F387I/I390A/L749F/L750D/ | ++ | +++ | | ++ |
| | | N1040E/L1248D/L1377D/V1439E | | | | |
| 1039/1040 | P617D | L171P/S260T/E306G/S332P/F387I/I390A/P617D/L749F/ | ++ | ++ | + | + |
| | | L750D/L1248D/L1377D/V1439E | | | | |
| 1041/1042 | Q611E | L171P/S260T/E306G/S332P/F387I/I390A/Q611E/L749F/ | ++ | ++ | ++ | + |
| | | L750D/L1248D/L1377D/V1439E | | | | |
| 1043/1044 | V620P | L171P/S260T/E306G/S332P/F387I/I390A/V620P/L749F/ | ++ | ++ | | + |
| | | L750D/L1248D/L1377D/V1439E | | | | |
| 1045/1046 | P617G | L171P/S260T/E306G/S332P/F387I/I390A/P617G/L749F/ | ++ | ++ | + | ++ |
| | | L750D/L1248D/L1377D/V1439E | | | | |
| 1047/1048 | A488G | L171P/S260T/E306G/S332P/F387I/I390A/A488G/L749F/ | | | + | + |
| | | L750D/L1248D/L1377D/V1439E | | | | |
| 1049/1050 | P1192D | L171P/S260T/E306G/S332P/F387I/I390A/L749F/L750D/ | ++ | ++ | | + |
| | | P1192D/L1248D/L1377D/V1439E | | | | |
| 1051/1052 | S1194P | L171P/S260T/E306G/S332P/F387I/I390A/L749F/L750D/ | ++ | ++ | | + |
| | | S1194P/L1248D/L1377D/V1439E | | | | |
| 1053/1054 | G619E | L171P/S260T/E306G/S332P/F387I/I390A/G619E/L749F/ | ++ | ++ | ++ | + |
| | | L750D/L1248D/L1377D/V1439E | | | | |
| 1055/1056 | P615D | L171P/S260T/E306G/S332P/F387I/I390A/P615D/L749F/ | + | + | + | + |
| | | L750D/L1248D/L1377D/V1439E | | | | |
| 1057/1058 | F16A | F16A/L171P/S260T/E306G/S332P/F387I/I390A/L749F/ | ++ | ++ | ++ | + |
| | | L750D/L1248D/L1377D/V1439E | | | | |
| 1059/1060 | F802S | L171P/S260T/E306G/S332P/F387I/I390A/L749F/L750D/ | ++ | + | ++ | + |
| | | F802S/L1248D/L1377D/V1439E | | | | |
| 1061/1062 | K818V | L171P/S260T/E306G/S332P/F387I/I390A/L749F/L750D/ | + | ++ | + | + |
| | | K818V/L1248D/L1377D/V1439E | | | | |
| 1063/1064 | L211K | L171P/L211K/S260T/E306G/S332P/F387I/I390A/L749F/ | + | ++ | + | ++ |
| | | L750D/L1248D/L1377D/V1439E | | | | |
| 1065/1066 | M836W | L171P/S260T/E306G/S332P/F387I/I390A/L749F/M836W/ | ++ | ++ | | ++ |
| | | L750D/L1248D/L1377D/V1439E | | | | |
| 1067/1068 | F555R | L171P/S260T/E306G/S332P/F387I/I390A/F555R/L749F/ | ++ | ++ | | ++ |
| | | L750D/L1248D/L1377D/V1439E | | | | |
| 1069/1070 | V536A | L171P/S260T/E306G/S332P/F387I/I390A/V536A/L749F/ | + | ++ | + | + |
| | | L750D/L1248D/L1377D/V1439E | | | | |
| 1071/1072 | I527A | L171P/S260T/E306G/S332P/F387I/I390A/I527A/L749F/ | | + | | + |
| | | L750D/L1248D/L1377D/V1439E | | | | |
| 1073/1074 | F555E | L171P/S260T/E306G/S332P/F387I/I390A/F555E/L749F/ | ++ | ++ | + | ++ |
| | | L750D/L1248D/L1377D/V1439E | | | | |
| 1075/1076 | H841W | L171P/S260T/E306G/S332P/F387I/I390A/L749F/L750D/ | + | ++ | | + |
| | | H841W/L1248D/L1377D/V1439E | | | | |
| 1077/1078 | M320Q | L171P/S260T/E306G/M320Q/S332P/F387I/I390A/L749F/ | + | ++ | + | + |
| | | L750D/L1248D/L1377D/V1439E | | | | |
| 1079/1080 | S837G | L171P/S260T/E306G/S332P/F387I/I390A/L749F/L750D/ | ++ | | ++ | + |
| | | S837G/L1248D/L1377D/V1439E | | | | |
| 1081/1082 | M558R | L171P/S260T/E306G/S332P/F387I/I390A/M558R/L749F/ | + | + | | |
| | | L750D/L1248D/L1377D/V1439E | | | | |
| 1083/1084 | F555G | L171P/S260T/E306G/S332P/F387I/I390A/F555G/L749F/ | + | ++ | + | + |
| | | L750D/L1248D/L1377D/V1439E | | | | |
| 1085/1086 | L211R | L171P/L211R/S260T/E306G/S332P/F387I/I390A/L749F/ | + | + | + | + |
| | | L750D/L1248D/L1377D/V1439E | | | | |
| 1087/1088 | V64G | V64G/L171P/S260T/E306G/S332P/F387I/I390A/L749F/ | ++ | + | + | + |
| | | L750D/L1248D/L1377D/V1439E | | | | |
| 1089/1090 | M320R | L171P/S260T/E306G/M320R/S332P/F387I/I390A/L749F/ | ++ | +++ | ++ | ++ |
| | | L750D/L1248D/L1377D/V1439E | | | | |
| 1091/1092 | T533Y | L171P/S260T/E306G/S332P/F387I/I390A/T533Y/L749F/ | + | | ++ | ++ |
| | | L750D/L1248D/L1377D/V1439E | | | | |
| 1093/1094 | L914V | L171P/S260T/E306G/S332P/F387I/I390A/L749F/L750D/ | ++ | + | ++ | ++ |
| | | L914V/L1248D/L1377D/V1439E | | | | |
| 1095/1096 | F16V | F16V/L171P/S260T/E306G/S332P/F387I/I390A/L749F/ | ++ | + | ++ | ++ |
| | | L750D/L1248D/L1377D/V1439E | | | | |
| 1097/1098 | I1283E | L171P/S260T/E306G/S332P/F387I/I390A/L749F/L750D/ | ++ | | ++ | + |
| | | L1248D/I1283E/L1377D/V1439E | | | | |
| 1099/1100 | V536M | L171P/S260T/E306G/S332P/F387I/I390A/V536M/L749F/ | + | | ++ | + |
| | | L750D/L1248D/L1377D/V1439E | | | | |
| 1101/1102 | F312L | L171P/S260T/E306G/F312L/S332P/F387I/I390A/L749F/ | ++ | ++ | ++ | ++ |
| | | L750D/L1248D/L1377D/V1439E | | | | |
| 1103/1104 | M681N | L171P/S260T/E306G/S332P/F387I/I390A/M681N/L749F/ | ++ | +++ | + | ++ |
| | | L750D/L1248D/L1377D/V1439E | | | | |
| 1105/1106 | S838T | L171P/S260T/E306G/S332P/F387I/I390A/L749F/L750D/ | ++ | + | ++ | + |
| | | S838T/L1248D/L1377D/V1439E | | | | |
| 1107/1108 | V536R | L171P/S260T/E306G/S332P/F387I/I390A/V536R/L749F/ | + | + | ++ | + |
| | | L750D/L1248D/L1377D/V1439E | | | | |
| 1109/1110 | R15G | R15G/L171P/S260T/E306G/S332P/F387I/I390A/L749F/ | ++ | + | ++ | + |
| | | L750D/L1248D/L1377D/V1439E | | | | |
| 1111/1112 | V536P | L171P/S260T/E306G/S332P/F387I/I390A/V536P/L749F/ | ++ | ++ | | ++ |
| | | L750D/L1248D/L1377D/V1439E | | | | |
| 1113/1114 | M558W | L171P/S260T/E306G/S332P/F387I/I390A/M558W/L749F/ | ++ | ++ | ++ | + |
| | | L750D/L1248D/L1377D/V1439E | | | | |
| 1115/1116 | M558E | L171P/S260T/E306G/S332P/F387I/I390A/M558E/L749F/ | ++ | | ++ | + |
| | | L750D/L1248D/L1377D/V1439E | | | | |
| 1117/1118 | L1427Y | L171P/S260T/E306G/S332P/F387I/I390A/L749F/L750D/ | ++ | ++ | ++ | ++ |
| | | L1248D/L1377D/L1427Y/V1439E | | | | |
| 1119/1120 | S839G | L171P/S260T/E306G/S332P/F387I/I390A/L749F/L750D/ | | | ++ | + |
| | | S839G/L1248D/L1377D/V1439E | | | | |
| 1121/1122 | F18L | F18L/L171P/S260T/E306G/S332P/F387I/I390A/L749F/ | + | ++ | + | + |
| | | L750D/L1248D/L1377D/V1439E | | | | |
| 1123/1124 | L13G | L13G/L171P/S260T/E306G/S332P/F387I/I390A/L749F/ | ++ | ++ | + | + |
| | | L750D/L1248D/L1377D/V1439E | | | | |
| 1125/1126 | K1210M | L171P/S260T/E306G/S332P/F387I/I390A/L749F/L750D/ | + | | ++ | + |
| | | K1210M/L1248D/L1377D/V1439E | | | | |
| 1127/1128 | H1156G | L171P/S260T/E306G/S332P/F387I/I390A/L749F/L750D/ | + | ++ | ++ | + |
| | | H1156G/L1248D/L1377D/V1439E | | | | |
| 1129/1130 | M558L | L171P/S260T/E306G/S332P/F387I/I390A/M558L/L749F/ | + | ++ | | |
| | | L750D/L1248D/L1377D/V1439E | | | | |
| 1131/1132 | S839K | L171P/S260T/E306G/S332P/F387I/I390A/L749F/L750D/ | + | ++ | ++ | ++ |
| | | S839K/L1248D/L1377D/V1439E | | | | |
| 1133/1134 | L211P | L171P/L211P/S260T/E306G/S332P/F387I/I390A/L749F/ | + | | + | + |
| | | L750D/L1248D/L1377D/V1439E | | | | |
| 1135/1136 | L211G | L171P/L211G/S260T/E306G/S332P/F387I/I390A/L749F/ | + | | + | + |
| | | L750D/L1248D/L1377D/V1439E | | | | |
| 1137/1138 | L211Q | L171P/L211Q/S260T/E306G/S332P/F387I/I390A/L749F/ | ++ | ++ | + | ++ |
| | | L750D/L1248D/L1377D/V1439E | | | | |
| 1139/1140 | F16D | F16D/L171P/S260T/E306G/S332P/F387I/I390A/L749F/ | ++ | + | + | ++ |
| | | L750D/L1248D/L1377D/V1439E | | | | |
| 1141/1142 | M320G | L171P/S260T/E306G/M320G/S332P/F387I/I390A/L749F/ | + | + | | |
| | | L750D/L1248D/L1377D/V1439E | | | | |
| 1143/1144 | Y683W | L171P/S260T/E306G/S332P/F387I/I390A/Y683W/L749F/ | ++ | ++ | | + |
| | | L750D/L1248D/L1377D/V1439E | | | | |
| 1145/1146 | Y24V | Y24V/L171P/S260T/E306G/S332P/F387I/I390A/L749F/ | ++ | ++ | | + |
| | | L750D/L1248D/L1377D/V1439E | | | | |
| 1147/1148 | V534I | L171P/S260T/E306G/S332P/F387I/I390A/V534I/L749F/ | ++ | ++ | + | ++ |
| | | L750D/L1248D/L1377D/V1439E | | | | |
| 1149/1150 | M681A | L171P/S260T/E306G/S332P/F387I/I390A/M681A/L749F/ | ++ | ++ | + | + |
| | | L750D/L1248D/L1377D/V1439E | | | | |
| 1151/1152 | P840G | L171P/S260T/E306G/S332P/F387I/I390A/L749F/L750D/ | + | + | | + |
| | | P840G/L1248D/L1377D/V1439E | | | | |
| 1153/1154 | F1160R | L171P/S260T/E306G/S332P/F387I/I390A/L749F/L750D/ | ++ | + | | + |
| | | F1160R/L1248D/L1377D/V1439E | | | | |
| 1155/1156 | M681C | L171P/S260T/E306G/S332P/F387I/I390A/M681C/L749F/ | + | ++ | ++ | + |
| | | L750D/L1248D/L1377D/V1439E | | | | |
| 1157/1158 | F328Q | L171P/S260T/E306G/F328Q/S332P/F387I/I390A/L749F/ | | | + | + |
| | | L750D/L1248D/L1377D/V1439E | | | | |
| 1159/1160 | R15S | R15S/L171P/S260T/E306G/S332P/F387I/I390A/L749F/ | + | + | + | + |
| | | L750D/L1248D/L1377D/V1439E | | | | |
| 1161/1162 | V536K | L171P/S260T/E306G/S332P/F387I/I390A/V536K/L749F/ | + | + | + | + |
| | | L750D/L1248D/L1377D/V1439E | | | | |
| 1163/1164 | V536G | L171P/S260T/E306G/S332P/F387I/I390A/V536G/L749F/ | ++ | ++ | | + |
| | | L750D/L1248D/L1377D/V1439E | | | | |
| 1165/1166 | T533R | L171P/S260T/E306G/S332P/F387I/I390A/T533R/L749F/ | + | ++ | + | ++ |
| | | L750D/L1248D/L1377D/V1439E | | | | |
| 1167/1168 | M681E | L171P/S260T/E306G/S332P/F387I/I390A/M681E/L749F/ | ++ | | ++ | ++ |
| | | L750D/L1248D/L1377D/V1439E | | | | |
| 1169/1170 | V536W | L171P/S260T/E306G/S332P/F387I/I390A/V536W/L749F/ | ++ | ++ | | ++ |
| | | L750D/L1248D/L1377D/V1439E | | | | |
| 1171/1172 | H1156N | L171P/S260T/E306G/S332P/F387I/I390A/L749F/L750D/ | + | + | ++ | ++ |
| | | H1156N/L1248D/L1377D/V1439E | | | | |
| 1173/1174 | F16P | F16P/L171P/S260T/E306G/S332P/F387I/I390A/L749F/ | ++ | + | + | + |
| | | L750D/L1248D/L1377D/V1439E | | | | |
| 1175/1176 | Q817A | L171P/S260T/E306G/S332P/F387I/I390A/L749F/L750D/ | ++ | ++ | | ++ |
| | | Q817A/L1248D/L1377D/V1439E | | | | |
| 1177/1178 | F18R | F18R/L171P/S260T/E306G/S332P/F387I/I390A/L749F/ | ++ | ++ | | + |
| | | L750D/L1248D/L1377D/V1439E | | | | |
| 1179/1180 | Y551W | L171P/S260T/E306G/S332P/F387I/I390A/Y551W/L749F/ | ++ | + | + | + |
| | | L750D/L1248D/L1377D/V1439E | | | | |
| 1181/1182 | F555T | L171P/S260T/E306G/S332P/F387I/I390A/F555T/L749F/ | + | ++ | | + |
| | | L750D/L1248D/L1377D/V1439E | | | | |
| 1183/1184 | M681G | L171P/S260T/E306G/S332P/F387I/I390A/M681G/L749F/ | ++ | +++ | ++ | + |
| | | L750D/L1248D/L1377D/V1439E | | | | |
| 1185/1186 | H212A | L171P/H212A/S260T/E306G/S332P/F387I/I390A/L749F/ | + | ++ | + | + |
| | | L750D/L1248D/L1377D/V1439E | | | | |
| 1187/1188 | F16G | F16G/L171P/S260T/E306G/S332P/F387I/I390A/L749F/ | ++ | + | ++ | + |
| | | L750D/L1248D/L1377D/V1439E | | | | |
| 1189/1190 | S838G | L171P/S260T/E306G/S332P/F387I/I390A/L749F/L750D/ | + | + | | + |
| | | S838G/L1248D/L1377D/V1439E | | | | |
| 1191/1192 | M681R | L171P/S260T/E306G/S332P/F387I/I390A/M681R/L749F/ | + | + | + | + |
| | | L750D/L1248D/L1377D/V1439E | | | | |
| 1193/1194 | V393G | L171P/S260T/E306G/S332P/F387I/I390A/V393G/L749F/ | ++ | ++ | ++ | |
| | | L750D/L1248D/L1377D/V1439E | | | | |
| 1195/1196 | V393G/A747D/K752G/ | L171P/S260T/E306G/S332P/F387I/I390A/V393G/A747D/ | ++ | ++ | ++ | ++ |
| | Q811F/K819V/ | L749F/L750D/K752G/Q811F/K819V/Q923M/T1148N | | | | |
| | Q923M/T1148N | L1248D/L1377D/V1439E | | | | |
| 1197/1198 | V393G/S751Y/ | L171P/S260T/E306G/S332P/F387I/I390A/V393G/L749F/ | ++ | +++ | + | + |
| | I775G | L750D/S751Y/I775G/L1248D/L1377D/V1439E | | | | |
| 1199/1200 | V63Q/V393G/K732W/ | V63Q/L171P/S260T/E306G/S332P/F387I/I390A/V393G/ | ++ | +++ | ++ | ++ |
| | I775G/T1148N | K732W/L749F/L750D/I775G/T1148N/L1248D/L1377D/ | | | | |
| | | V1439E | | | | |
| 1201/1202 | V393G/K732W/ | L171P/S260T/E306G/S332P/F387I/I390A/V393G/K732W/ | ++ | ++ | ++ | ++ |
| | T1148N | L749F/L750D/T1148N/L1248D/L1377D/V1439E | | | | |
| 1203/1204 | I775G/T1148N | L171P/S260T/E306G/S332P/F387I/I390A/L749F/L750D/ | ++ | +++ | ++ | ++ |
| | | I775G/T1148N/L1248D/L1377D/V1439E | | | | |
| 1205/1206 | V393G/K752L/N753D/ | L171P/S260T/E306G/S332P/F387I/I390A/V393G/L749F/ | ++ | +++ | + | ++ |
| | I775G/I793G/ | L750D/K752L/N753D/I775G/I793G/M797W/L1248D/ | | | | |
| | M797W | L1377D/V1439E | | | | |
| 1207/1208 | V393G/Q923M | L171P/S260T/E306G/S332P/F387I/I390A/V393G/L749F/ | ++ | ++ | ++ | + |
| | | L750D/Q923M/L1248D/L1377D/V1439E | | | | |
| 1209/1210 | V393G/Q923M/ | L171P/S260T/E306G/S332P/F387I/I390A/V393G/L749F/ | ++ | +++ | ++ | + |
| | T1148N | L750D/Q923M/T1148N/L1248D/L1377D/V1439E | | | | |
| 1211/1212 | V393G/A747D/ | L171P/S260T/E306G/S332P/F387I/I390A/V393G/A747D/ | ++ | ++ | ++ | ++ |
| | S751Y | L749F/L750D/S751Y/L1248D/L1377D/V1439E | | | | |
| 1213/1214 | V393G/A747D/N753D/ | L171P/S260T/E306G/S332P/F387I/I390A/V393G/A747D/ | ++ | +++ | ++ | ++ |
| | I775G/T1148N | L749F/L750D/N753D/I775G/T1148N/L1248D/L1377D/ | | | | |
| | | V1439E | | | | |
| 1215/1216 | K752L/Q923M/ | L171P/S260T/E306G/S332P/F387I/I390A/L749F/L750D/ | ++ | ++ | ++ | + |
| | M1051R | K752L/Q923M/M1051R/L1248D/L1377D/V1439E | | | | |
| 1217/1218 | V63Q/I775G | V63Q/L171P/S260T/E306G/S332P/F387I/I390A/L749F/ | ++ | +++ | ++ | ++ |
| | | L750D/I775G/L1248D/L1377D/V1439E | | | | |
| 1219/1220 | V63Q/V393G/K752L/ | V63Q/L171P/S260T/E306G/S332P/F387I/I390A/V393G/ | ++ | ++ | + | + |
| | I775G/I793G/ | L749F/L750D/K752L/I775G/I793G/M1051R/T1053R | | | | |
| | M1051R/T1053R | L1248D/L1377D/V1439E | | | | |
| 1221/1222 | K752G/N753D/I775G/ | L171P/S260T/E306G/S332P/F387I/I390A/L749F/L750D/ | ++ | ++ | ++ | ++ |
| | I793G/K819V | K752G/N753D/I775G/I793G/K819V/L1248D/L1377D/ | | | | |
| | | V1439E | | | | |
| 1223/1224 | V63Q/M1051R | V63Q/L171P/S260T/E306G/S332P/F387I/I390A/L749F/ | ++ | +++ | ++ | ++ |
| | | L750D/M1051R/L1248D/L1377D/V1439E | | | | |
| 1225/1226 | V63Q/A747D/K752G/ | V63Q/L171P/S260T/E306G/S332P/F387I/I390A/A747D/ | ++ | +++ | ++ | ++ |
| | N753D/I775G/Q811F/ | L749F/L750D/K752G/N753D/I775G/Q811F/K819V/ | | | | |
| | K819V/Q923M/ | Q923M/M1051R L1248D/L1377D/V1439E | | | | |
| | M1051R | | | | | |
| 1227/1228 | V393G/K752G/ | L171P/S260T/E306G/S332P/F387I/I390A/V393G/L749F/ | + | ++ | ++ | ++ |
| | I787W | L750D/K752G/I787W/L1248D/L1377D/V1439E | | | | |
| 1229/1230 | S751Y | L171P/S260T/E306G/S332P/F387I/I390A/L749F/L750D/ | + | + | + | ++ |
| | | S751Y/L1248D/L1377D/V1439E | | | | |
| 1231/1232 | V63Q/T1148N | V63Q/L171P/S260T/E306G/S332P/F387I/I390A/L749F/ | ++ | ++ | ++ | ++ |
| | | L750D/T1148N/L1248D/L1377D/V1439E | | | | |
| 1233/1234 | V63H/A747D/K752G/ | V63H/L171P/S260T/E306G/S332P/F387I/I390A/A747D/ | ++ | + | ++ | ++ |
| | N753D/K819V/ | L749F/L750D/K752G/N753D/K819V/Q923M/T1148N | | | | |
| | Q923M/T1148N | L1248D/L1377D/V1439E | | | | |
| 1235/1236 | T1148N | L171P/S260T/E306G/S332P/F387I/I390A/L749F/L750D/ | + | ++ | + | ++ |
| | | T1148N/L1248D/L1377D/V1439E | | | | |
| 1237/1238 | A747D/K752L/ | L171P/S260T/E306G/S332P/F387I/I390A/A747D/L749F/ | ++ | ++ | + | ++ |
| | N753D/I793G/ | L750D/K752L/N753D/I793G/Q811F/L1248D/L1377 | | | | |
| | Q811F | V1439E | | | | |
| 1239/1240 | V63Q | V63Q/L171P/S260T/E306G/S332P/F387I/I390A/L749F/ | ++ | +++ | ++ | ++ |
| | | L750D/L1248D/L1377D/V1439E | | | | |
| 1241/1242 | M797W/T1148N | L171P/S260T/E306G/S332P/F387I/I390A/L749F/L750D/ | + | + | ++ | + |
| | | M797W/T1148N/L1248D/L1377D/V1439E | | | | |
| 1243/1244 | S751Y/K752L/ | L171P/S260T/E306G/S332P/F387I/I390A/L749F/L750D/ | ++ | +++ | ++ | ++ |
| | N753D/M1051R | S751Y/K752L/N753D/M1051R/L1248D/L1377D/V1439E | | | | |
| 1245/1246 | V63Q/K732W/M1051R | V63Q/L171P/S260T/E306G/S332P/F387I/I390A/K732W/ | ++ | ++ | ++ | + |
| | | L749F/L750D/M1051R/L1248D/L1377D/V1439E | | | | |
| 1247/1248 | K752G/M797W/S815I/ | L171P/S260T/E306G/S332P/F387I/I390A/L749F/L750D/ | ++ | +++ | ++ | ++ |
| | Q923M/M1051R/ | K752G/M797W/S815I/Q923M/M1051R/T1148N | | | | |
| | T1148N | L1248D/L1377D/V1439E | | | | |
| 1249/1250 | A747D/N753D | L171P/S260T/E306G/S332P/F387I/I390A/A747D/L749F/ | ++ | ++ | ++ | ++ |
| | | L750D/N753D/L1248D/L1377D/V1439E | | | | |

All activities were determined relative to the reference polypeptide of SEQ ID NO: 840. Levels of increased activity are defined as follows:
“+” 0.9 to 1.1,
“++” >1.1,
“+++” >2

### Example 9

### FVIII Variants Derived from SEQ ID NO: 1200

In this Example, experiments conducted to determine FVIII variant activity by assaying the protein activity after a challenge is described. These variants were tested for FVIII activity after no incubation and after incubation in Hemophilia A patient plasma (George King Bio-Medical, Inc.), as described in Example 3. Variants were tested using the chromogenic and clotting assays, as described in Example 3. Results are shown in Tables 9.1 and 9.2 for variants expressed using Expi293F cells or HepG2 cells.

TABLE 9.1
Activity of FVII Variants Relative to SEQ ID NO: 1200

| | | | | (Expi293F) |
|---|---|---|---|---|
| | | | (Expi293F) | Chromogenic |
| | | | Unchallenged | Activity after |
| | | | Chromogenic | Plasma |
| | Amino Acid | | Activity | Challenge |
| | Differences | Amino Acid | FIOPC | FIOPC |
| SEQ ID | (Relative to | Differences | Relative to | Relative to |
| NO: | SEQ ID NO: | (Relative to | SEQ ID NO: | SEQ ID NO: |
| (nt/aa) | 1200) | SEQ ID NO: 2) | 1200 | 1200 |
| 1199/1200 | | V63Q/L171P/S260T/E306G/S332P/ | + | + |
| | | F387I/I390A/V393G/K732W/L749F/ | | |
| | | L750D/I775G/T1148N/L1248D/L1377D/ | | |
| | | V1439E | | |
| 1251/1252 | M681A | V63Q/L171P/S260T/E306G/S332P/ | + | +++ |
| | | F387I/I390A/V393G/M681A/K732W/ | | |
| | | L749F/L750D/I775G/T1148N/L1248D/ | | |
| | | L1377D/V1439E | | |
| 1253/1254 | L622D | V63Q/L171P/S260T/E306G/S332P/ | + | + |
| | | F387I/I390A/V393G/L622D/K732W/ | | |
| | | L749F/L750D/I775G/T1148N/L1248D/ | | |
| | | L1377D/V1439E | | |
| 1255/1256 | P617G | V63Q/L171P/S260T/E306G/S332P/ | ++ | + |
| | | F387I/I390A/V393G/P617G/K732W/ | | |
| | | L749F/L750D/I775G/T1148N/L1248D/ | | |
| | | L1377D/V1439E | | |
| 1257/1258 | M558L | V63Q/L171P/S260T/E306G/S332P/ | + | +++ |
| | | F387I/I390A/V393G/M558L/K732W/ | | |
| | | L749F/L750D/I775G/T1148N/L1248D/ | | |
| | | L1377D/V1439E | | |
| 1259/1260 | V536K | V63Q/L171P/S260T/E306G/S332P/ | + | +++ |
| | | F387I/I390A/V393G/V536K/K732W/ | | |
| | | L749F/L750D/I775G/T1148N/L1248D/ | | |
| | | L1377D/V1439E | | |
| 1261/1262 | M681G | V63Q/L171P/S260T/E306G/S332P/ | + | +++ |
| | | F387I/I390A/V393G/M681G/K732W/ | | |
| | | L749F/L750D/I775G/T1148N/L1248D/ | | |
| | | L1377D/V1439E | | |
| 1263/1264 | L211Q | V63Q/L171P/L211Q/S260T/E306G/ | + | + |
| | | S332P/F387I/I390A/V393G/K732W/ | | |
| | | L749F/L750D/I775G/T1148N/L1248D/ | | |
| | | L1377D/V1439E | | |
| 1265/1266 | M320R | V63Q/L171P/S260T/E306G/M320R/ | +++ | +++ |
| | | S332P/F387I/I390A/V393G/K732W/ | | |
| | | L749F/L750D/I775G/T1148N/L1248D/ | | |
| | | L1377D/V1439E | | |
| 1267/1268 | M558W | V63Q/L171P/S260T/E306G/S332P/ | + | +++ |
| | | F387I/I390A/V393G/M558W/K732W/ | | |
| | | L749F/L750D/I775G/T1148N/L1248D/ | | |
| | | L1377D/V1439E | | |
| 1269/1270 | S839K | V63Q/L171P/S260T/E306G/S332P/ | +++ | +++ |
| | | F387I/I390A/V393G/K732W/L749F/ | | |
| | | L750D/I775G/S839K/T1148N/L1248D/ | | |
| | | L1377D/V1439E | | |
| 1271/1272 | L211K | V63Q/L171P/L211K/S260T/E306G/ | ++ | + |
| | | S332P/F387I/I390A/V393G/K732W/ | | |
| | | L749F/L750D/I775G/T1148N/L1248D/ | | |
| | | L1377D/V1439E | | |
| 1273/1274 | M797W | V63Q/L171P/S260T/E306G/S332P/ | + | +++ |
| | | F387I/I390A/V393G/K732W/L749F/ | | |
| | | L750D/I775G/M797W/T1148N/L1248D/ | | |
| | | L1377D/V1439E | | |
| 1275/1276 | T533R | V63Q/L171P/S260T/E306G/S332P/ | + | ++ |
| | | F387I/I390A/V393G/T533R/K732W/ | | |
| | | L749F/L750D/I775G/T1148N/L1248D/ | | |
| | | L1377D/V1439E | | |
| 1277/1278 | S751A | V63Q/L171P/S260T/E306G/S332P/ | + | ++ |
| | | F387I/I390A/V393G/K732W/L749F/ | | |
| | | L750D/S751A/I775G/T1148N/L1248D/ | | |
| | | L1377D/V1439E | | |
| 1279/1280 | A303T | V63Q/L171P/S260T/A303T/E306G/ | +++ | + |
| | | S332P/F387I/I390A/V393G/K732W/ | | |
| | | L749F/L750D/I775G/T1148N/L1248D/ | | |
| | | L1377D/V1439E | | |
| 1281/1282 | T951V | V63Q/L171P/S260T/E306G/S332P/ | ++ | + |
| | | F387I/I390A/V393G/K732W/L749F/ | | |
| | | L750D/T951V/I775G/T1148N/L1248D/ | | |
| | | L1377D/V1439E | | |
| 1283/1284 | G164E | V63Q/G164E/L171P/S260T/E306G/ | ++ | +++ |
| | | S332P/F387I/I390A/V393G/K732W/ | | |
| | | L749F/L750D/I775G/T1148N/L1248D/ | | |
| | | L1377D/V1439E | | |
| 1285/1286 | K818G | V63Q/L171P/S260T/E306G/S332P/ | + | ++ |
| | | F387I/I390A/V393G/K732W/L749F/ | | |
| | | L750D/I775G/K818G/T1148N/L1248D/ | | |
| | | L1377D/V1439E | | |
| 1287/1288 | E807L | V63Q/L171P/S260T/E306G/S332P/ | +++ | + |
| | | F387I/I390A/V393G/K732W/L749F/ | | |
| | | L750D/I775G/E807L/T1148N/L1248D/ | | |
| | | L1377D/V1439E | | |
| 1289/1290 | K799A | V63Q/L171P/S260T/E306G/S332P/ | ++ | + |
| | | F387I/I390A/V393G/K732W/L749F/ | | |
| | | L750D/I775G/K799A/T1148N/L1248D/ | | |
| | | L1377D/V1439E | | |
| 1291/1292 | G735S | V63Q/L171P/S260T/E306G/S332P/ | ++ | ++ |
| | | F387I/I390A/V393G/K732W/G735S/ | | |
| | | L749F/L750D/I775G/T1148N/L1248D/ | | |
| | | L1377D/V1439E | | |
| 1293/1294 | S794R | V63Q/L171P/S260T/E306G/S332P/ | + | ++ |
| | | F387I/I390A/V393G/K732W/L749F/ | | |
| | | L750D/I775G/S794R/T1148N/L1248D/ | | |
| | | L1377D/V1439E | | |
| 1295/1296 | E926V | V63Q/L171P/S260T/E306G/S332P/ | + | ++ |
| | | F387I/I390A/V393G/K732W/L749F/ | | |
| | | L750D/I775G/E926V/T1148N/L1248D/ | | |
| | | L1377D/V1439E | | |
| 1297/1298 | A755P | V63Q/L171P/S260T/E306G/S332P/ | + | +++ |
| | | F387I/I390A/V393G/K732W/L749F/ | | |
| | | L750D/A755P/I775G/T1148N/L1248D/ | | |
| | | L1377D/V1439E | | |
| 1299/1300 | Q921W | V63Q/L171P/S260T/E306G/S332P/ | +++ | + |
| | | F387I/I390A/V393G/K732W/L749F/ | | |
| | | L750D/I775G/Q921W/T1148N/L1248D/ | | |
| | | L1377D/V1439E | | |
| 1301/1302 | D801A | V63Q/L171P/S260T/E306G/S332P/ | + | ++ |
| | | F387I/I390A/V393G/K732W/L749F/ | | |
| | | L750D/I775G/D801A/T1148N/L1248D/ | | |
| | | L1377D/V1439E | | |
| 1303/1304 | E1094S | V63Q/L171P/S260T/E306G/S332P/ | + | ++ |
| | | F387I/I390A/V393G/K732W/L749F/ | | |
| | | L750D/I775G/E1094S/T1148N/L1248D/ | | |
| | | L1377D/V1439E | | |
| 1305/1306 | L968A | V63Q/L171P/S260T/E306G/S332P/ | ++ | + |
| | | F387I/I390A/V393G/K732W/L749F/ | | |
| | | L750D/I775G/L968A/T1148N/L1248D/ | | |
| | | L1377D/V1439E | | |
| 1307/1308 | K929Q | V63Q/L171P/S260T/E306G/S332P/ | ++ | ++ |
| | | F387I/I390A/V393G/K732W/L749F/ | | |
| | | L750D/I775G/K929Q/T1148N/L1248D/ | | |
| | | L1377D/V1439E | | |
| 1309/1310 | G924W | V63Q/L171P/S260T/E306G/S332P/ | ++ | +++ |
| | | F387I/I390A/V393G/K732W/L749F/ | | |
| | | L750D/I775G/G924W/T1148N/L1248D/ | | |
| | | L1377D/V1439E | | |
| 1311/1312 | E807P | V63Q/L171P/S260T/E306G/S332P/ | + | ++ |
| | | F387I/I390A/V393G/K732W/L749F/ | | |
| | | L750D/I775G/E807P/T1148N/L1248D/ | | |
| | | L1377D/V1439E | | |
| 1313/1314 | F698D | V63Q/L171P/S260T/E306G/S332P/ | + | +++ |
| | | F387I/I390A/V393G/F698D/K732W/ | | |
| | | L749F/L750D/I775G/T1148N/L1248D/ | | |
| | | L1377D/V1439E | | |
| 1315/1316 | S168G | V63Q/S168G/L171P/S260T/E306G/ | + | ++ |
| | | S332P/F387I/I390A/V393G/K732W/ | | |
| | | L749F/L750D/I775G/T1148N/L1248D/ | | |
| | | L1377D/V1439E | | |
| 1317/1318 | K929V | V63Q/L171P/S260T/E306G/S332P/ | ++ | + |
| | | F387I/I390A/V393G/K732W/L749F/ | | |
| | | L750D/I775G/K929V/T1148N/L1248D/ | | |
| | | L1377D/V1439E | | |
| 1319/1320 | K818A | V63Q/L171P/S260T/E306G/S332P/ | + | + |
| | | F387I/I390A/V393G/K732W/L749F/ | | |
| | | L750D/I775G/K818A/T1148N/L1248D/ | | |
| | | L1377D/V1439E | | |
| 1321/1322 | K818S | V63Q/L171P/S260T/E306G/S332P/ | + | + |
| | | F387I/I390A/V393G/K732W/L749F/ | | |
| | | L750D/I775G/K818S/T1148N/L1248D/ | | |
| | | L1377D/V1439E | | |
| 1323/1324 | R581K | V63Q/L171P/S260T/E306G/S332P/ | + | + |
| | | F387I/I390A/V393G/R581K/K732W/ | | |
| | | L749F/L750D/I775G/T1148N/L1248D/ | | |
| | | L1377D/V1439E | | |
| 1325/1326 | K818P | V63Q/L171P/S260T/E306G/S332P/ | + | +++ |
| | | F387I/I390A/V393G/K732W/L749F/ | | |
| | | L750D/I775G/K818P/T1148N/L1248D/ | | |
| | | L1377D/V1439E | | |
| 1327/1328 | S916K | V63Q/L171P/S260T/E306G/S332P/ | + | + |
| | | F387I/I390A/V393G/K732W/L749F/ | | |
| | | L750D/I775G/S916K/T1148N/L1248D/ | | |
| | | L1377D/V1439E | | |
| 1329/1330 | F698S | V63Q/L171P/S260T/E306G/S332P/ | + | + |
| | | F387I/I390A/V393G/F698S/K732W/ | | |
| | | L749F/L750D/I775G/T1148N/L1248D/ | | |
| | | L1377D/V1439E | | |
| 1331/1332 | A303G | V63Q/L171P/S260T/A303G/E306G/ | +++ | + |
| | | S332P/F387I/I390A/V393G/K732W/ | | |
| | | L749F/L750D/I775G/T1148N/L1248D/ | | |
| | | L1377D/V1439E | | |
| 1333/1334 | S916C | V63Q/L171P/S260T/E306G/S332P/ | + | + |
| | | F387I/I390A/V393G/K732W/L749F/ | | |
| | | L750D/I775G/S916C/T1148N/L1248D/ | | |
| | | L1377D/V1439E | | |
| 1335/1336 | T1428G | V63Q/L171P/S260T/E306G/S332P/ | + | ++ |
| | | F387I/I390A/V393G/K732W/L749F/ | | |
| | | L750D/I775G/T1148N/L1248D/L1377D/ | | |
| | | T1428G/V1439E | | |
| 1337/1338 | Y805L | V63Q/L171P/S260T/E306G/S332P/ | +++ | ++ |
| | | F387I/I390A/V393G/K732W/L749F/ | | |
| | | L750D/I775G/Y805L/T1148N/L1248D/ | | |
| | | L1377D/V1439E | | |
| 1339/1340 | G924M | V63Q/L171P/S260T/E306G/S332P/ | ++ | + |
| | | F387I/I390A/V393G/K732W/L749F/ | | |
| | | L750D/I775G/G924M/T1148N/L1248D/ | | |
| | | L1377D/V1439E | | |
| 1341/1342 | L1114M | V63Q/L171P/S260T/E306G/S332P/ | ++ | + |
| | | F387I/I390A/V393G/K732W/L749F/ | | |
| | | L750D/I775G/L1114M/T1148N/L1248D/ | | |
| | | L1377D/V1439E | | |
| 1343/1344 | T311G | V63Q/L171P/S260T/E306G/T311G/ | +++ | + |
| | | S332P/F387I/I390A/V393G/K732W/ | | |
| | | L749F/L750D/I775G/T1148N/L1248D/ | | |
| | | L1377D/V1439E | | |
| 1345/1346 | Y737N | V63Q/L171P/S260T/E306G/S332P/ | + | + |
| | | F387I/I390A/V393G/K732W/Y737N/ | | |
| | | L749F/L750D/I775G/T1148N/L1248D/ | | |
| | | L1377D/V1439E | | |
| 1347/1348 | Y805V | V63Q/L171P/S260T/E306G/S332P/ | ++ | ++ |
| | | F387I/I390A/V393G/K732W/L749F/ | | |
| | | L750D/I775G/Y805V/T1148N/L1248D/ | | |
| | | L1377D/V1439E | | |
| 1349/1350 | D801I | V63Q/L171P/S260T/E306G/S332P/ | + | ++ |
| | | F387I/I390A/V393G/K732W/L749F/ | | |
| | | L750D/I775G/D801I/T1148N/L1248D/ | | |
| | | L1377D/V1439E | | |
| 1351/1352 | S751D | V63Q/L171P/S260T/E306G/S332P/ | + | + |
| | | F387I/I390A/V393G/K732W/L749F/ | | |
| | | L750D/S751D/I775G/T1148N/L1248D/ | | |
| | | L1377D/V1439E | | |
| 1353/1354 | S693T | V63Q/L171P/S260T/E306G/S332P/ | ++ | + |
| | | F387I/I390A/V393G/S693T/K732W/ | | |
| | | L749F/L750D/I775G/T1148N/L1248D/ | | |
| | | L1377D/V1439E | | |
| 1355/1356 | K818E | V63Q/L171P/S260T/E306G/S332P/ | + | + |
| | | F387I/I390A/V393G/K732W/L749F/ | | |
| | | L750D/I775G/K818E/T1148N/L1248D/ | | |
| | | L1377D/V1439E | | |
| 1357/1358 | K929A | V63Q/L171P/S260T/E306G/S332P/ | ++ | + |
| | | F387I/I390A/V393G/K732W/L749F/ | | |
| | | L750D/I775G/K929A/T1148N/L1248D/ | | |
| | | L1377D/V1439E | | |
| 1359/1360 | T311E | V63Q/L171P/S260T/E306G/T311E/ | +++ | + |
| | | S332P/F387I/I390A/V393G/K732W/ | | |
| | | L749F/L750D/I775G/T1148N/L1248D/ | | |
| | | L1377D/V1439E | | |
| 1361/1362 | Y737L | V63Q/L171P/S260T/E306G/S332P/ | + | ++ |
| | | F387I/I390A/V393G/K732W/Y737L/ | | |
| | | L749F/L750D/I775G/T1148N/L1248D/ | | |
| | | L1377D/V1439E | | |
| 1363/1364 | E809W | V63Q/L171P/S260T/E306G/S332P/ | +++ | + |
| | | F387I/I390A/V393G/K732W/L749F/ | | |
| | | L750D/I775G/E809W/T1148N/L1248D/ | | |
| | | L1377D/V1439E | | |
| 1365/1366 | E757A | V63Q/L171P/S260T/E306G/S332P/ | +++ | + |
| | | F387I/I390A/V393G/K732W/L749F/ | | |
| | | L750D/E757A/I775G/T1148N/L1248D/ | | |
| | | L1377D/V1439E | | |
| 1367/1368 | A1099G | V63Q/L171P/S260T/E306G/S332P/ | + | + |
| | | F387I/I390A/V393G/K732W/L749F/ | | |
| | | L750D/I775G/A1099G/T1148N/L1248D/ | | |
| | | L1377D/V1439E | | |
| 1369/1370 | K799G | V63Q/L171P/S260T/E306G/S332P/ | + | ++ |
| | | F387I/I390A/V393G/K732W/L749F/ | | |
| | | L750D/I775G/K799G/T1148N/L1248D/ | | |
| | | L1377D/V1439E | | |
| 1371/1372 | S916I | V63Q/L171P/S260T/E306G/S332P/ | + | + |
| | | F387I/I390A/V393G/K732W/L749F/ | | |
| | | L750D/I775G/S916I/T1148N/L1248D/ | | |
| | | L1377D/V1439E | | |
| 1373/1374 | Y805G | V63Q/L171P/S260T/E306G/S332P/ | +++ | + |
| | | F387I/I390A/V393G/K732W/L749F/ | | |
| | | L750D/I775G/Y805G/T1148N/L1248D/ | | |
| | | L1377D/V1439E | | |
| 1375/1376 | Y737F | V63Q/L171P/S260T/E306G/S332P/ | + | ++ |
| | | F387I/I390A/V393G/K732W/Y737F/ | | |
| | | L749F/L750D/I775G/T1148N/L1248D/ | | |
| | | L1377D/V1439E | | |
| 1377/1378 | Y737M | V63Q/L171P/S260T/E306G/S332P/ | ++ | ++ |
| | | F387I/I390A/V393G/K732W/Y737M/ | | |
| | | L749F/L750D/I775G/T1148N/L1248D/ | | |
| | | L1377D/V1439E | | |
| 1379/1380 | K799P | V63Q/L171P/S260T/E306G/S332P/ | ++ | + |
| | | F387I/I390A/V393G/K732W/L749F/ | | |
| | | L750D/I775G/K799P/T1148N/L1248D/ | | |
| | | L1377D/V1439E | | |
| 1381/1382 | E807C | V63Q/L171P/S260T/E306G/S332P/ | ++ | + |
| | | F387I/I390A/V393G/K732W/L749F/ | | |
| | | L750D/I775G/E807C/T1148N/L1248D/ | | |
| | | L1377D/V1439E | | |
| 1383/1384 | S916E | V63Q/L171P/S260T/E306G/S332P/ | ++ | + |
| | | F387I/I390A/V393G/K732W/L749F/ | | |
| | | L750D/I775G/S916E/T1148N/L1248D/ | | |
| | | L1377D/V1439E | | |
| 1385/1386 | T676Q | V63Q/L171P/S260T/E306G/S332P/ | ++ | + |
| | | F387I/I390A/V393G/T676Q/K732W/ | | |
| | | L749F/L750D/I775G/T1148N/L1248D/ | | |
| | | L1377D/V1439E | | |
| 1387/1388 | R581V | V63Q/L171P/S260T/E306G/S332P/ | ++ | ++ |
| | | F387I/I390A/V393G/R581V/K732W/ | | |
| | | L749F/L750D/I775G/T1148N/L1248D/ | | |
| | | L1377D/V1439E | | |
| 1389/1390 | L968P | V63Q/L171P/S260T/E306G/S332P/ | ++ | +++ |
| | | F387I/I390A/V393G/K732W/L749F/ | | |
| | | L750D/I775G/L968P/T1148N/L1248D/ | | |
| | | L1377D/V1439E | | |
| 1391/1392 | A755Y | V63Q/L171P/S260T/E306G/S332P/ | ++ | ++ |
| | | F387I/I390A/V393G/K732W/L749F/ | | |
| | | L750D/A755Y/I775G/T1148N/L1248D/ | | |
| | | L1377D/V1439E | | |
| 1393/1394 | S916Q | V63Q/L171P/S260T/E306G/S332P/ | +++ | +++ |
| | | F387I/I390A/V393G/K732W/L749F/ | | |
| | | L750D/I775G/S916Q/T1148N/L1248D/ | | |
| | | L1377D/V1439E | | |
| 1395/1396 | G735D | V63Q/L171P/S260T/E306G/S332P/ | +++ | + |
| | | F387I/I390A/V393G/K732W/G735D/ | | |
| | | L749F/L750D/I775G/T1148N/L1248D/ | | |
| | | L1377D/V1439E | | |
| 1397/1398 | D740G | V63Q/L171P/S260T/E306G/S332P/ | +++ | +++ |
| | | F387I/I390A/V393G/K732W/D740G/ | | |
| | | L749F/L750D/I775G/T1148N/L1248D/ | | |
| | | L1377D/V1439E | | |
| 1399/1400 | F698Y | V63Q/L171P/S260T/E306G/S332P/ | + | + |
| | | F387I/I390A/V393G/K732W/F698Y/ | | |
| | | L749F/L750D/I775G/T1148N/L1248D/ | | |
| | | L1377D/V1439E | | |
| 1401/1402 | E757G | V63Q/L171P/S260T/E306G/S332P/ | ++ | ++ |
| | | F387I/I390A/V393G/K732W/L749F/ | | |
| | | L750D/E757G/I775G/T1148N/L1248D/ | | |
| | | L1377D/V1439E | | |
| 1403/1404 | D791T | V63Q/L171P/S260T/E306G/S332P/ | +++ | + |
| | | F387I/I390A/V393G/K732W/L749F/ | | |
| | | L750D/I775G/D791T/T1148N/L1248D/ | | |
| | | L1377D/V1439E | | |
| 1405/1406 | L1114A | V63Q/L171P/S260T/E306G/S332P/ | + | ++ |
| | | F387I/I390A/V393G/K732W/L749F/ | | |
| | | L750D/I775G/L1114A/T1148N/L1248D/ | | |
| | | L1377D/V1439E | | |
| 1407/1408 | G735V | V63Q/L171P/S260T/E306G/S332P/ | + | ++ |
| | | F387I/I390A/V393G/K732W/G735V/ | | |
| | | L749F/L750D/I775G/T1148N/L1248D/ | | |
| | | L1377D/V1439E | | |

All activities were determined relative to the reference polypeptide of SEQ ID NO: 1200. Levels of increased activity are defined as follows: “+” 0.9 to 1.1, “++” >1.1, “+++” >2

TABLE 9.2
Activity of FVIII Variants Relative to SEQ ID NO: 1200

| | | | | (Expi293F) | | (HepG2) | |
|---|---|---|---|---|---|---|---|
| | | | (Expi293F) | Chromogenic | (HepG2) | Chromogenic | (Expi293F) |
| | | | Unchallenged | Activity | Unchallenged | Activity after | Unchallenged |
| | | | Chromogenic | after Plasma | Chromogenic | Plasma | Clotting |
| | Amino Acid | | Activity | Challenge | Activity | Challenge | Activity |
| | Differences | Amino Acid | FIOPC | FIOPC | FIOPC | FIOPC | FIOPC |
| SEQ ID | (Relative to | Differences | Relative to | Relative to | Relative to | Relative to | Relative to |
| NO: | SEQ ID NO: | (Relative to | SEQ ID NO: | SEQ ID NO: | SEQ ID NO: | SEQ ID NO: | SEQ ID NO: |
| (nt/aa) | 1200) | SEQ ID NO: 2) | 1200 | 1200 | 1200 | 1200 | 1200 |
| 1199/1200 | | V63Q/L171P/S260T/E306G/S332P/ | + | + | + | + | + |
| | | F387I/I390A/V393G/K732W/L749F/ | | | | | |
| | | L750D/I775G/T1148N/L1248D/ | | | | | |
| | | L1377D/V1439E | | | | | |
| 1409/1410 | V536P | V63Q/L171P/S260T/E306G/S332P/ | ++ | + | ++ | ++ | + |
| | | F387I/I390A/V393G/V536P/K732W/ | | | | | |
| | | L749F/L750D/I775G/T1148N/ | | | | | |
| | | L1248D/L1377D/V1439E | | | | | |
| 1411/1412 | L211R/V536K/ | V63Q/L171P/L211R/S260T/E306G/ | ++ | ++ | + | ++ | + |
| | L622D/M681A | S332P/F387I/I390A/V393G/V536K/ | | | | | |
| | | L622D/M681A/K732W/L749F/L750D/ | | | | | |
| | | I775G/T1148N/L1248D/L1377D/ | | | | | |
| | | V1439E | | | | | |
| 1413/1414 | V536K/M558L | V63Q/L171P/S260T/E306G/S332P/ | ++ | + | ++ | + | + |
| | | F387I/I390A/V393G/V536K/M558L/ | | | | | |
| | | K732W/L749F/L750D/I775G/ | | | | | |
| | | T1148N/L1248D/L1377D/V1439E | | | | | |
| 1415/1416 | L211R/V536R/ | V63Q/L171P/L211R/S260T/E306G/ | ++ | ++ | ++ | ++ | + |
| | F555G/P617G/ | S332P/F387I/I390A/V393G/V536R/ | | | | | |
| | W732K | F555G/P617G/L749F/L750D/I775G/ | | | | | |
| | | T1148N/L1248D/L1377D/V1439E | | | | | |
| 1417/1418 | L211R/M681R/ | V63Q/L171P/L211R/S260T/E306G/ | ++ | | ++ | + | + |
| | W732K | S332P/F387I/I390A/V393G/M681R/ | | | | | |
| | | L749F/L750D/I775G/T1148N/ | | | | | |
| | | L1248D/L1377D/V1439E | | | | | |
| 1419/1420 | L211R/V536K/ | V63Q/L171P/L211R/S260T/E306G/ | ++ | ++ | + | + | + |
| | F555E | S332P/F387I/I390A/V393G/V536K/ | | | | | |
| | | F555E/K732W/L749F/L750D/I775G/ | | | | | |
| | | T1148N/L1248D/L1377D/V1439E | | | | | |
| 1421/1422 | V536R | V63Q/L171P/S260T/E306G/S332P/ | ++ | ++ | + | + | + |
| | | F387I/I390A/V393G/V536R/K732W/ | | | | | |
| | | L749F/L750D/I775G/T1148N/ | | | | | |
| | | L1248D/L1377D/V1439E | | | | | |
| 1423/1424 | F555E | V63Q/L171P/S260T/E306G/S332P/ | ++ | ++ | ++ | ++ | ++ |
| | | F387I/I390A/V393G/F555E/K732W/ | | | | | |
| | | L749F/L750D/I775G/T1148N/ | | | | | |
| | | L1248D/L1377D/V1439E | | | | | |
| 1425/1426 | L211R/V536P | V63Q/L171P/L211R/S260T/E306G/ | ++ | ++++ | ++ | ++ | + |
| | | S332P/F387I/I390A/V393G/V536P/ | | | | | |
| | | K732W/L749F/L750D/I775G/ | | | | | |
| | | T1148N/L1248D/L1377D/V1439E | | | | | |
| 1427/1428 | L211R/H212P | V63Q/L171P/L211R/H212P/S260T/ | ++ | + | ++ | ++ | + |
| | | E306G/S332P/F387I/I390A/V393G/ | | | | | |
| | | K732W/L749F/L750D/I775G/ | | | | | |
| | | T1148N/L1248D/L1377D/V1439E | | | | | |
| 1429/1430 | L211R/V536K | V63Q/L171P/L211R/S260T/E306G/ | + | + | + | ++ | + |
| | | S332P/F387I/I390A/V393G/V536K/ | | | | | |
| | | K732W/L749F/L750D/I775G/ | | | | | |
| | | T1148N/L1248D/L1377D/V1439E | | | | | |
| 1431/1432 | L211R/V536K/ | V63Q/L171P/L211R/S260T/E306G/ | ++ | + | + | ++ | + |
| | P617G/M681A/ | S332P/F387I/I390A/V393G/ | | | | | |
| | W732K | V536K/P617G/M681A/L749F/ | | | | | |
| | | L750D/I775G/T1148N/L1248D/ | | | | | |
| | | L1377D/V1439E | | | | | |
| 1433/1434 | L211R | V63Q/L171P/L211R/S260T/E306G/ | ++ | ++ | ++ | ++ | ++ |
| | | S332P/F387I/I390A/V393G/K732W/ | | | | | |
| | | L749F/L750D/I775G/T1148N/ | | | | | |
| | | L1248D/L1377D/V1439E | | | | | |
| 1435/1436 | L211R/V536P/ | V63Q/L171P/L211R/S260T/E306G/ | ++ | + | ++ | ++ | ++ |
| | M558L/P617G/ | S332P/F387I/I390A/V393G/V536P/ | | | | | |
| | M681A | M558L/P617G/M681A/K732W/ | | | | | |
| | | L749F/L750D/I775G/T1148N/L1248D/ | | | | | |
| | | L1377D/V1439E | | | | | |
| 1437/1438 | F555G/W732K | V63Q/L171P/S260T/E306G/S332P/ | ++ | + | ++ | ++ | + |
| | | F3871/I390A/V393G/F555G/L749F/ | | | | | |
| | | L750D/I775G/T1148N/L1248D/ | | | | | |
| | | L1377D/V1439E | | | | | |
| 1439/1440 | V536P/F555E/ | V63Q/L171P/S260T/E306G/S332P/ | ++ | +++ | ++ | ++ | + |
| | M558L/P617G/ | F387I/I390A/V393G/V536P/F555E/ | | | | | |
| | W732K | M558L/P617G/L749F/L750D/I775G/ | | | | | |
| | | T1148N/L1248D/L1377D/V1439E | | | | | |
| 1441/1442 | V536K/P617G/ | V63Q/L171P/S260T/E306G/S332P/ | ++ | ++ | + | ++ | + |
| | M681A/W732K | F387I/I390A/V393G/V536K/P617G/ | | | | | |
| | | M681A/L749F/L750D/I775G/ | | | | | |
| | | NT1148/L1248D/L1377D/V1439E | | | | | |
| 1443/1444 | V536K/F555G | V63Q/L171P/S260T/E306G/S332P/ | ++ | + | ++ | ++ | + |
| | | F387I/I390A/V393G/V536K/F555G/ | | | | | |
| | | K732W/L749F/L750D/I775G/T1148N/ | | | | | |
| | | L1248D/L1377D/V1439E | | | | | |
| 1445/1446 | V536P/L622D/ | V63Q/L171P/S260T/E306G/S332P/ | ++ | ++ | | + | + |
| | M681A | F3871/I390A/V393G/V536P/L622D/ | | | | | |
| | | M681A/K732W/L749F/L750D/I775G/ | | | | | |
| | | T1148N/L1248D/L1377D/V1439E | | | | | |
| 1447/1448 | W732K | V63Q/L171P/S260T/E306G/S332P/ | ++ | | ++ | ++ | + |
| | | F387I/I390A/V393G/L749F/L750D/ | | | | | |
| | | I775G/T1148N/L1248D/L1377D/ | | | | | |
| | | V1439E | | | | | |
| 1449/1450 | H212A/M320Q/ | V63Q/L171P/H212A/S260T/E306G/ | ++ | ++ | ++ | ++ | + |
| | T533R/P617E/ | M320Q/S332P/F387I/I390A/V393G/ | | | | | |
| | M681G | T533R/P617E/M681G/K732W/L749F/ | | | | | |
| | | L750D/I775G/T1148N/L1248D/ | | | | | |
| | | L1377D/V1439E | | | | | |
| 1451/1452 | L211K/F312L/ | V63Q/L171P/L211K/S260T/E306G/ | ++ | + | ++ | ++ | ++ |
| | T533R/V534I/ | F312L/S332P/F387I/I390A/V393G/ | | | | | |
| | P617D/G619E/ | T533R/V534I/P617D/G619E/M681N/ | | | | | |
| | M681N/S839K | K732W/L749F/L750D/I775G/S839K/ | | | | | |
| | | T1148N/L1248D/L1377D/V1439E | | | | | |
| 1453/1454 | H212A | V63Q/L171P/H212A/S260T/E306G/ | + | + | + | + | + |
| | | S332P/F387I/I390A/V393G/K732W/ | | | | | |
| | | L749F/L750D/I775G/T1148N/ | | | | | |
| | | L1248D/L1377D/V1439E | | | | | |
| 1455/1456 | L211K/H212A/ | V63Q/L171P/L211K/H212A/S260T/ | +++ | ++ | ++ | ++ | ++ |
| | M320Q/G619E/ | E306G/M320Q/S332P/F387I/I390A/ | | | | | |
| | M681G/S839K | V393G/G619E/M681G/K732W/ | | | | | |
| | | L749F/L750D/I775G/S839K/T1148N/ | | | | | |
| | | L1248D/L1377D/V1439E | | | | | |
| 1457/1458 | M797W/S839K | V63Q/L171P/S260T/E306G/S332P/ | + | ++ | + | + | + |
| | | F387I/I390A/V393G/K732W/L749F/ | | | | | |
| | | L750D/I775G/M797W/S839K/ | | | | | |
| | | T1148N/L1248D/L1377D/V1439E | | | | | |
| 1459/1460 | H212A/F312L/ | V63Q/L171P/H212A/S260T/E306G/ | ++ | +++ | ++ | ++ | + |
| | M320Q/T533R/ | F312L/M320Q/S332P/F387I/I390A/ | | | | | |
| | V534I/P617E/ | V393G/T533R/V534I/P617E/M681G/ | | | | | |
| | M681G | K732W/L749F/L750D/I775G/T1148N/ | | | | | |
| | | L1248D/L1377D/V1439E | | | | | |
| 1461/1462 | M558W/G619E/ | V63Q/L171P/S260T/E306G/S332P/ | ++ | ++ | ++ | +++ | ++ |
| | M681G | F387I/I390A/V393G/M558W/G619E/ | | | | | |
| | | M681G/K732W/L749F/L750D/I775G/ | | | | | |
| | | T1148N/L1248D/L1377D/V1439E | | | | | |
| 1463/1464 | M320Q/M558W/ | V63Q/L171P/S260T/E306G/M320Q/ | ++ | ++ | ++ | ++ | + |
| | M681G | S332P/F387I/I390A/V393G/ | | | | | |
| | | M558W/M681G/K732W/L749F/L750D/ | | | | | |
| | | I775G/T1148N/L1248D/L1377D/ | | | | | |
| | | V1439E | | | | | |
| 1465/1466 | H212A/M320Q/ | V63Q/L171P/H212A/S260T/E306G/ | ++ | ++ | ++ | ++ | + |
| | V534I/G619E/ | M320Q/S332P/F387I/I390A/V393G/ | | | | | |
| | M681G/M797W/ | V534I/G619E/M681G/K732W/L749F/ | | | | | |
| | S839K | L750D/I775G/M797W/S839K/T1148N/ | | | | | |
| | | L1248D/L1377D/V1439E | | | | | |
| 1467/1468 | M320R/M558W/ | V63Q/L171P/S260T/E306G/M320R/ | ++ | ++++ | + | ++ | + |
| | G619E/W732K/ | S332P/F387I/I390A/V393G/M558W/ | | | | | |
| | S839K | G619E/L749F/L750D/I775G/S839K/ | | | | | |
| | | T1148N/L1248D/L1377D/V1439E | | | | | |
| 1469/1470 | L211K/H212A/ | V63Q/L171P/L211K/H212A/S260T/ | +++ | ++++ | ++ | ++ | + |
| | F312L/V534I/ | E306G/F312L/S332P/F387I/I390A/ | | | | | |
| | M558W/G619E/ | V393G/V534I/M558W/G619E/M681N/ | | | | | |
| | M681N/W732K/ | L749F/L750D/I775G/ | | | | | |
| | S839K | S839K/T1148N/L1248D/L1377D/ | | | | | |
| | | V1439E | | | | | |
| 1471/1472 | M320R/V534I/ | V63Q/L171P/S260T/E306G/M320R/ | +++ | ++ | ++ | ++ | ++ |
| | M558R/G619E/ | S332P/F387I/I390A/V393G/V534I/ | | | | | |
| | W732K/M797W | M558R/G619E/L749F/L750D/I775G/ | | | | | |
| | | M797W/T1148N/L1248D/L1377D/ | | | | | |
| | | V1439E | | | | | |
| 1473/1474 | M558W/P617D/ | V63Q/L171P/S260T/E306G/S332P/ | ++ | ++ | ++ | ++ | ++ |
| | G619E/M681N | F387I/I390A/V393G/M558W/P617D/ | | | | | |
| | | G619E/M681N/K732W/L749F/L750D/ | | | | | |
| | | I775G/T1148N/L1248D/L1377D/ | | | | | |
| | | V1439E | | | | | |
| 1475/1476 | L211Q/V534I/ | V63Q/L171P/L211Q/S260T/E306G/ | ++ | ++ | ++ | ++ | ++ |
| | M558W/Q611E/ | S332P/F387I/I390A/V393G/V534I/ | | | | | |
| | M681N | M558W/Q611E/M681N/K732W/L749F/ | | | | | |
| | | L750D/I775G/T1148N/L1248D/ | | | | | |
| | | L1377D/V1439E | | | | | |
| 1477/1478 | L211K/M320R/ | V63Q/L171P/L211K/S260T/E306G/ | +++ | ++ | ++ | +++ | ++ |
| | P617E/G619E/ | M320R/S332P/F387I/I390A/V393G/ | | | | | |
| | M681G | P617E/G619E/M681G/K732W/L749F/ | | | | | |
| | | L750D/I775G/T1148N/L1248D/ | | | | | |
| | | L1377D/V1439E | | | | | |
| 1479/1480 | M558W/P617D/ | V63Q/L171P/S260T/E306G/S332P/ | ++ | ++ | ++ | ++ | ++ |
| | M681G/M797W | F387I/I390A/V393G/M558W/P617D/ | | | | | |
| | | M681G/K732W/L749F/L750D/I775G/ | | | | | |
| | | M797W/T1148N/L1248D/L1377D/ | | | | | |
| | | V1439E | | | | | |
| 1481/1482 | M320R/V534I/ | V63Q/L171P/S260T/E306G/M320R/ | +++ | +++ | ++ | ++ | ++ |
| | G619E/W732K | S332P/F387I/I390A/V393G/ | | | | | |
| | | V534I/G619E//L749F/L750D/I775G/ | | | | | |
| | | T1148N/L1248D/L1377D/V1439E | | | | | |
| 1483/1484 | L211K/G619E/ | V63Q/L171P/L211K/S260T/E306G/ | +++ | ++ | ++ | ++ | ++ |
| | M681N | S332P/F387I/I390A/V393G/ | | | | | |
| | | G619E/M681N/K732W/L749F/L750D/ | | | | | |
| | | I775G/T1148N/L1248D/L1377D/ | | | | | |
| | | V1439E | | | | | |
| 1485/1486 | L211K/H212A/ | V63Q/L171P/L211K/H212A/S260T/ | ++ | +++ | ++ | ++ | + |
| | V534I/M558W/ | E306G/S332P/F387I/I390A/V393G/ | | | | | |
| | G619E/W732K/ | V534I/M558W/G619E/L749F/L750D/ | | | | | |
| | S839K | I775G/S839K/T1148N/L1248D/ | | | | | |
| | | L1377D/V1439E | | | | | |
| 1487/1488 | H212A/M320R/ | V63Q/L171P/H212A/S260T/E306G/ | +++ | +++++ | +++ | ++ | ++ |
| | G619E/M681N/ | M320R/S332P/F387I/I390A/V393G/ | | | | | |
| | M797W/S839K | G619E/M681N/K732W/L749F/L750D/ | | | | | |
| | | I775G/M797W/S839K/T1148N/ | | | | | |
| | | L1248D/L1377D/V1439E | | | | | |
| 1489/1490 | H212A/M320R/ | V63Q/L171P/H212A/S260T/E306G/ | ++ | +++ | ++ | ++ | + |
| | Q611E/S839K | M320R/S332P/F387I/I390A/V393G/ | | | | | |
| | | Q611E/K732W/L749F/L750D/I775G/ | | | | | |
| | | S839K/T1148N/L1248D/L1377D/ | | | | | |
| | | V1439E | | | | | |
| 1491/1492 | M320Q/M558W/ | V63Q/L171P/S260T/E306G/M320Q/ | +++ | +++ | +++ | +++ | ++ |
| | G619E/M681N | S332P/F387I/I390A/V393G/M558W/ | | | | | |
| | | G619E/M681N/K732W/L749F/L750D/ | | | | | |
| | | I775G/T1148N/L1248D/L1377D/ | | | | | |
| | | V1439E | | | | | |
| 1493/1494 | F312L/T533R/ | V63Q/L171P/S260T/E306G/F312L/ | ++ | +++ | ++ | ++ | ++ |
| | P617D/M681N/ | S332P/F387I/I390A/V393G/T533R/ | | | | | |
| | M797W/S839K | P617D/M681N/K732W/L749F/L750D/ | | | | | |
| | | I775G/M797W/S839K/T1148N/ | | | | | |
| | | L1248D/L1377D/V1439E | | | | | |
| 1495/1496 | M558W/Q611E/ | V63Q/L171P/S260T/E306G/S332P/ | ++ | +++ | + | ++ | ++ |
| | M681N | F387I/I390A/V393G/M558W/Q611E/ | | | | | |
| | | M681N/K732W/L749F/L750D/I775G/ | | | | | |
| | | T1148N/L1248D/L1377D/V1439E | | | | | |
| 1497/1498 | F312L | V63Q/L171P/S260T/E306G/F312L/ | ++ | ++ | ++ | ++ | + |
| | | S332P/F387I/I390A/V393G/K732W/ | | | | | |
| | | L749F/L750D/I775G/T1148N/ | | | | | |
| | | L1248D/L1377D/V1439E | | | | | |
| 1499/1500 | L211Q/M320R/ | V63Q/L171P/L211Q/S260T/E306G/ | ++ | ++ | ++ | ++ | + |
| | M558W/G619E/ | M320R/S332P/F387I/I390A/V393G/ | | | | | |
| | M681G/W732K/ | M558W/G619E/M681G/L749F/L750D/ | | | | | |
| | M797W/S839K | I775G/M797W/S839K/T1148N/ | | | | | |
| | | L1248D/L1377D/V1439E | | | | | |
| 1501/1502 | M320Q/M558W/ | V63Q/L171P/S260T/E306G/M320Q/ | ++ | + | ++ | ++ | + |
| | W732K/M797W | S332P/F387I/I390A/V393G/M558W/ | | | | | |
| | | L749F/L750D/I775G/M797W/T1148N/ | | | | | |
| | | L1248D/L1377D/V1439E | | | | | |
| 1503/1504 | T533R/M558W/ | V63Q/L171P/S260T/E306G/S332P/ | ++ | + | ++ | ++ | + |
| | Q611E/G619E/ | F387I/I390A/V393G/T533R/M558W/ | | | | | |
| | F716L/W732K/ | Q611E/G619E/F716L//L749F/L750D/ | | | | | |
| | S839K | I775G/S839K/T1148N/L1248D/ | | | | | |
| | | L1377D/V1439E | | | | | |
| 1505/1506 | F312L/M681G | V63Q/L171P/S260T/E306G/F312L/ | +++ | ++ | ++ | +++ | ++ |
| | | S332P/F387I/I390A/V393G/M681G/ | | | | | |
| | | K732W/L749F/L750D/I775G/T1148N/ | | | | | |
| | | L1248D/L1377D/V1439E | | | | | |
| 1507/1508 | M320Q/V534I/ | V63Q/L171P/S260T/E306G/M320Q/ | ++ | +++ | ++ | ++ | ++ |
| | G619E/M681N | S332P/F387I/I390A/V393G/V534I/ | | | | | |
| | | G619E/M681N/K732W/L749F/L750D/ | | | | | |
| | | I775G/T1148N/L1248D/L1377D/ | | | | | |
| | | V1439E | | | | | |
| 1509/1510 | L211K/M320Q/ | V63Q/L171P/L211K/S260T/E306G/ | + | ++ | + | + | + |
| | T533R/V536L/ | M320Q/S332P/F387I/I390A/V393G/ | | | | | |
| | G619E | T533R/V536L/G619E/K732W/L749F/ | | | | | |
| | | L750D/I775G/T1148N/L1248D/ | | | | | |
| | | L1377D/V1439E | | | | | |
| 1511/1512 | M558W/P617E/ | V63Q/L171P/S260T/E306G/S332P/ | ++ | ++ | ++ | ++ | ++ |
| | M681N/S839K | F387I/I390A/V393G/M558W/P617E/ | | | | | |
| | | M681N/K732W/L749F/L750D/I775G/ | | | | | |
| | | S839K/T1148N/L1248D/L1377D/ | | | | | |
| | | V1439E | | | | | |
| 1513/1514 | H212A/M320R/ | V63Q/L171P/H212A/S260T/E306G/ | +++ | ++++ | ++ | ++ | + |
| | V534I/M558W/ | M320R/S332P/F387I/I390A/V393G/ | | | | | |
| | M681G | V534I/M558W/M681G/K732W/L749F/ | | | | | |
| | | L750D/I775G/T1148N/L1248D/ | | | | | |
| | | L1377D/V1439E | | | | | |
| 1515/1516 | T533R/V534I/ | V63Q/L171P/S260T/E306G/S332P/ | ++ | ++ | + | ++ | + |
| | M681N/S839K | F387I/I390A/V393G/T533R/V534I/ | | | | | |
| | | M681N/K732W/L749F/L750D/I775G/ | | | | | |
| | | S839K/T1148N/L1248D/L1377D/ | | | | | |
| | | V1439E | | | | | |
| 1517/1518 | H212A/V534I/ | V63Q/L171P/H212A/S260T/E306G/ | +++ | +++ | ++ | +++ | ++ |
| | G619E/M681G | S332P/F387I/I390A/V393G/V534I/ | | | | | |
| | | G619E/M681G/K732W/L749F/L750D/ | | | | | |
| | | I775G/T1148N/L1248D/L1377D/ | | | | | |
| | | V1439E | | | | | |
| 1519/1520 | P617E/G619E/ | V63Q/L171P/S260T/E306G/S332P/ | ++ | ++ | + | ++ | ++ |
| | M681G | F387I/I390A/V393G/P617E/G619E/ | | | | | |
| | | M681G/K732W/L749F/L750D/I775G/ | | | | | |
| | | T1148N/L1248D/L1377D/V1439E | | | | | |
| 1521/1522 | L211Q/H212A/ | V63Q/L171P/L211Q/H212A/S260T/ | +++ | +++ | ++ | ++ | ++ |
| | M320R/M558W/ | E306G/M320R/S332P/F387I/I390A/ | | | | | |
| | P617E/M681N | V393G/M558W/P617E/M681N/K732W/ | | | | | |
| | | L749F/L750D/I775G/T1148N/ | | | | | |
| | | L1248D/L1377D/V1439E | | | | | |
| 1523/1524 | H212A/V534I/ | V63Q/L171P/H212A/S260T/E306G/ | ++ | ++ | ++ | ++ | ++ |
| | M558W/M681N/ | S332P/F387I/I390A/V393G/V534I/ | | | | | |
| | M797W | M558W/M681N/K732W/L749F/L750D/ | | | | | |
| | | I775G/M797W/T1148N/L1248D/ | | | | | |
| | | L1377D/V1439E | | | | | |
| 1525/1526 | H212A/M320R/ | V63Q/L171P/H212A/S260T/E306G/ | +++ | +++ | ++ | ++ | ++ |
| | V534I | M320R/S332P/F387I/I390A/V393G/ | | | | | |
| | | V534I/K732W/L749F/L750D/I775G/ | | | | | |
| | | T1148N/L1248D/L1377D/V1439E | | | | | |
| 1527/1528 | L211K/F312L/ | V63Q/L171P/L211K/S260T/E306G/ | ++ | ++ | + | + | ++ |
| | G619E | F312L/S332P/F387I/I390A/V393G/ | | | | | |
| | | G619E/K732W/L749F/L750D/I775G/ | | | | | |
| | | T1148N/L1248D/L1377D/V1439E | | | | | |
| 1529/1530 | H212A/F312L/ | V63Q/L171P/H212A/S260T/E306G/ | ++ | ++ | ++ | ++ | + |
| | G619E/S839K | F312L/S332P/F387I/I390A/V393G/ | | | | | |
| | | G619E/K732W/L749F/L750D/I775G/ | | | | | |
| | | S839K/T1148N/L1248D/L1377D/ | | | | | |
| | | V1439E | | | | | |
| 1531/1532 | T210P/H212A/ | V63Q/L171P/T210P/H212A/S260T/ | ++ | +++ | ++ | ++ | + |
| | M320R/M558W/ | E306G/M320R/S332P/F387I/I390A/ | | | | | |
| | M681G | V393G/M558W/M681G/K732W/L749F/ | | | | | |
| | | L750D/I775G/T1148N/L1248D/ | | | | | |
| | | L1377D/V1439E | | | | | |
| 1533/1534 | H212A/F312L/ | V63Q/L171P/H212A/S260T/E306G/ | +++ | ++ | ++ | +++ | ++ |
| | V534I/M681N/ | F312L/S332P/F387I/I390A/V393G/ | | | | | |
| | M797W/S839K | V534I/M681N/K732W/L749F/L750D/ | | | | | |
| | | I775G/M797W/S839K/T1148N/ | | | | | |
| | | L1248D/L1377D/V1439E | | | | | |
| 1535/1536 | L211K/M320R/ | V63Q/L171P/L211K/S260T/E306G/ | ++ | ++ | ++ | ++ | ++ |
| | M558W/S839K | M320R/S332P/F387I/I390A/V393G/ | | | | | |
| | | M558W/K732W/L749F/L750D/I775G/ | | | | | |
| | | S839K/T1148N/L1248D/L1377D/ | | | | | |
| | | V1439E | | | | | |
| 1537/1538 | G924Q | V63Q/L171P/S260T/E306G/S332P/ | + | + | ++ | ++ | + |
| | | F387I/I390A/V393G/K732W/L749F/ | | | | | |
| | | L750D/I775G/G924Q/T1148N/L1248D/ | | | | | |
| | | L1377D/V1439E | | | | | |
| 1539/1540 | S916L | V63Q/L171P/S260T/E306G/S332P/ | + | + | + | + | ++ |
| | | F387I/I390A/V393G/K732W/L749F/ | | | | | |
| | | L750D/I775G/S916L/T1148N/L1248D/ | | | | | |
| | | L1377D/V1439E | | | | | |
| 1541/1542 | E757T | V63Q/L171P/S260T/E306G/S332P/ | + | ++ | ++ | + | |
| | | F387I/I390A/V393G/K732W/L749F/ | | | | | |
| | | L750D/E757T/I775G/T1148N/L1248D/ | | | | | |
| | | L1377D/V1439E | | | | | |
| 1543/1544 | Y737K | V63Q/L171P/S260T/E306G/S332P/ | ++ | ++ | ++ | ++ | |
| | | F3871/I390A/V393G/K732W/Y737K/ | | | | | |
| | | L749F/L750D/I775G/T1148N/L1248D/ | | | | | |
| | | L1377D/V1439E | | | | | |
| 1545/1546 | K818V | V63Q/L171P/S260T/E306G/S332P/ | ++ | | ++ | ++ | + |
| | | F3871/I390A/V393G/K732W/L749F/ | | | | | |
| | | L750D/I775G/K818V/T1148N/L1248D/ | | | | | |
| | | L1377D/V1439E | | | | | |
| 1547/1548 | D801G | V63Q/L171P/S260T/E306G/S332P/ | ++ | ++ | ++ | ++ | ++ |
| | | F387I/I390A/V393G/K732W/L749F/ | | | | | |
| | | L750D/I775G/D801G/T1148N/L1248D/ | | | | | |
| | | L1377D/V1439E | | | | | |
| 1549/1550 | T294G | V63Q/L171P/S260T/T294G/E306G/ | ++ | ++ | | + | + |
| | | S332P/F387I/I390A/V393G/K732W/ | | | | | |
| | | L749F/L750D/I775G/T1148N/L1248D/ | | | | | |
| | | L1377D/V1439E | | | | | |
| 1551/1552 | K799S | V63Q/L171P/S260T/E306G/S332P/ | ++ | | ++ | ++ | ++ |
| | | F387I/I390A/V393G/K732W/L749F/ | | | | | |
| | | L750D/I775G/K799S/T1148N/L1248D/ | | | | | |
| | | L1377D/V1439E | | | | | |
| 1553/1554 | K799E | V63Q/L171P/S260T/E306G/S332P/ | + | | + | + | ++ |
| | | F387I/I390A/V393G/K732W/L749F/ | | | | | |
| | | L750D/I775G/K799E/T1148N/L1248D/ | | | | | |
| | | L1377D/V1439E | | | | | |
| 1555/1556 | E809G | V63Q/L171P/S260T/E306G/S332P/ | + | + | + | ++ | + |
| | | F387I/I390A/V393G/K732W/L749F/ | | | | | |
| | | L750D/I775G/E809G/T1148N/L1248D/ | | | | | |
| | | L1377D/V1439E | | | | | |
| 1557/1558 | D791M | V63Q/L171P/S260T/E306G/S332P/ | + | ++ | ++ | + | + |
| | | F387I/I390A/V393G/K732W/L749F/ | | | | | |
| | | L750D/I775G/D791M/T1148N/L1248D/ | | | | | |
| | | L1377D/V1439E | | | | | |
| 1559/1560 | Y737V | V63Q/L171P/S260T/E306G/S332P/ | ++ | ++ | ++ | ++ | + |
| | | F387I/I390A/V393G/K732W/Y737V/ | | | | | |
| | | L749F/L750D/I775G/T1148N/L1248D/ | | | | | |
| | | L1377D/V1439E | | | | | |
| 1561/1562 | Y805Q | V63Q/L171P/S260T/E306G/S332P/ | ++ | +++ | ++ | ++ | + |
| | | F387I/I390A/V393G/K732W/L749F/ | | | | | |
| | | L750D/I775G/Y805Q/T1148N/L1248D/ | | | | | |
| | | L1377D/V1439E | | | | | |
| 1563/1564 | K799Q | V63Q/L171P/S260T/E306G/S332P/ | + | + | + | ++ | + |
| | | F3871/I390A/V393G/K732W/L749F/ | | | | | |
| | | L750D/I775G/K799Q/T1148N/L1248D/ | | | | | |
| | | L1377D/V1439E | | | | | |
| 1565/1566 | Y737S | V63Q/L171P/S260T/E306G/S332P/ | + | ++ | + | + | + |
| | | F3871/I390A/V393G/K732W/Y737S/ | | | | | |
| | | L749F/L750D/I775G/T1148N/L1248D/ | | | | | |
| | | L1377D/V1439E | | | | | |
| 1567/1568 | S916M | V63Q/L171P/S260T/E306G/S332P/ | ++ | + | + | + | ++ |
| | | F387I/I390A/V393G/K732W/L749F/ | | | | | |
| | | L750D/I775G/S916M/T1148N/L1248D/ | | | | | |
| | | L1377D/V1439E | | | | | |
| 1569/1570 | R759S | V63Q/L171P/S260T/E306G/S332P/ | + | | | + | ++ |
| | | F387I/I390A/V393G/K732W/L749F/ | | | | | |
| | | L750D/R759S/I775G/T1148N/L1248D/ | | | | | |
| | | L1377D/V1439E | | | | | |
| 1571/1572 | K799V | V63Q/L171P/S260T/E306G/S332P/ | ++ | + | ++ | ++ | ++ |
| | | F3871/I390A/V393G/K732W/L749F/ | | | | | |
| | | L750D/I775G/K799V/T1148N/L1248D/ | | | | | |
| | | L1377D/V1439E | | | | | |
| 1573/1574 | D538V | V63Q/L171P/S260T/E306G/S332P/ | ++ | + | + | ++ | + |
| | | F387I/I390A/V393G/D538V/K732W/ | | | | | |
| | | L749F/L750D/I775G/T1148N/L1248D/ | | | | | |
| | | L1377D/V1439E | | | | | |
| 1575/1576 | E1109G | V63Q/L171P/S260T/E306G/S332P/ | ++ | ++ | + | + | ++ |
| | | F387I/I390A/V393G/K732W/L749F/ | | | | | |
| | | L750D/I775G/E1109G/T1148N/L1248D/ | | | | | |
| | | L1377D/V1439E | | | | | |
| 1577/1578 | S168R | V63Q/S168R/L171P/S260T/E306G/ | + | ++ | | + | ++ |
| | | S332P/F387I/I390A/V393G/K732W/ | | | | | |
| | | L749F/L750D/I775G/T1148N/L1248D/ | | | | | |
| | | L1377D/V1439E | | | | | |
| 1579/1580 | Q921R | V63Q/L171P/S260T/E306G/S332P/ | ++ | + | ++ | ++ | + |
| | | F3871/I390A/V393G/K732W/L749F/ | | | | | |
| | | L750D/I775G/Q921R/T1148N/L1248D/ | | | | | |
| | | L1377D/V1439E | | | | | |
| 1581/1582 | E926T | V63Q/L171P/S260T/E306G/S332P/ | ++ | | ++ | + | ++ |
| | | F387I/I390A/V393G/K732W/L749F/ | | | | | |
| | | L750D/I775G/E926T/T1148N/L1248D/ | | | | | |
| | | L1377D/V1439E | | | | | |
| 1583/1584 | S916T | V63Q/L171P/S260T/E306G/S332P/ | ++ | +++ | + | + | + |
| | | F387I/I390A/V393G/K732W/L749F/ | | | | | |
| | | L750D/I775G/S916T/T1148N/L1248D/ | | | | | |
| | | L1377D/V1439E | | | | | |
| 1585/1586 | Y737G | V63Q/L171P/S260T/E306G/S332P/ | ++ | + | ++ | + | + |
| | | F387I/I390A/V393G/K732W/Y737G/ | | | | | |
| | | L749F/L750D/I775G/T1148N/L1248D/ | | | | | |
| | | L1377D/V1439E | | | | | |
| 1587/1588 | D801R | V63Q/L171P/S260T/E306G/S332P/ | + | ++ | + | + | + |
| | | F387I/I390A/V393G/K732W/L749F/ | | | | | |
| | | L750D/I775G/D801R/T1148N/L1248D/ | | | | | |
| | | L1377D/V1439E | | | | | |

All activities were determined relative to the reference polypeptide of SEQ ID NO: 1200. Levels of increased activity are defined as follows:
“+” 0.9 to 1.1,
“++” >1.1,
“+++” >2,
“++++” >3.5,
”+++++” >5

### Example 10

### FVIII Variants Derived from SEQ ID NO: 1514

In this Example, experiments conducted to determine FVIII variant activity by assaying the protein activity after a challenge is described. These variants were tested for FVIII activity after no incubation and after incubation in Hemophilia A patient plasma (George King Bio-Medical, Inc.), as described in Example 3. Variants were tested using the chromogenic and clotting assays, as described in Example 3. Results are shown in Tables 10.1 and 10.2 for variants expressed using Expi293F cells or HepG2 cells.

TABLE 10.1
Activity of FVIII Variants Relative to SEQ ID NO: 1514

| | | | (Expi293F) | (Expi293F) |
|---|---|---|---|---|
| | | | Unchallenged | Chromogenic |
| | | | Chromogenic | Activity after |
| | Amino Acid | | Activity | Plasma |
| | Differences | Amino Acid | FIOPC | Challenge |
| SEQ ID | (Relative to | Differences | Relative to | FIOPC Relative |
| NO: | SEQ ID | (Relative to | SEQ ID NO: | to SEQ ID NO: |
| (nt/aa) | NO: 1514) | SEQ ID NO: 2) | 1514 | 1514 |
| 1513/1514 | | V63Q/L171P/H212A/S260T/E306G/ | + | + |
| | | M320R/S332P/F387I/I390A/V393G/ | | |
| | | V534I/M558W/M681G/K732W/L749F/ | | |
| | | L750D/I775G/T1148N/L1248D/L1377D/ | | |
| | | V1439E | | |
| 1589/1590 | R1042E | V63Q/L171P/H212A/S260T/E306G/ | + | + |
| | | M320R/S332P/F387I/I390A/V393G/ | | |
| | | V534I/M558W/M681G/K732W/L749F/ | | |
| | | L750D/I775G/R1042E/T1148N/L1248D/ | | |
| | | L1377D/V1439E | | |
| 1591/1592 | Y1041N | V63Q/L171P/H212A/S260T/E306G/ | + | + |
| | | M320R/S332P/F387I/I390A/V393G/ | | |
| | | V534I/M558W/M681G/K732W/L749F/ | | |
| | | L750D/I775G/Y1041N/T1148N/L1248D/ | | |
| | | L1377D/V1439E | | |
| 1593/1594 | M1051H | V63Q/L171P/H212A/S260T/E306G/ | + | + |
| | | M320R/S332P/F387I/I390A/V393G/ | | |
| | | V534I/M558W/M681G/K732W/L749F/ | | |
| | | L750D/I775G/M1051H/T1148N/L1248D/ | | |
| | | L1377D/V1439E | | |
| 1595/1596 | I1283H | V63Q/L171P/H212A/S260T/E306G/ | | + |
| | | M320R/S332P/F387I/I390A/V393G/ | | |
| | | V534I/M558W/M681G/K732W/L749F/ | | |
| | | L750D/I775G/T1148N/L1248D/I1283H/ | | |
| | | L1377D/V1439E | | |
| 1597/1598 | I1283T | V63Q/L171P/H212A/S260T/E306G/ | | + |
| | | M320R/S332P/F387I/I390A/V393G/ | | |
| | | V534I/M558W/M681G/K732W/L749F/ | | |
| | | L750D/I775G/T1148N/L1248D/I1283T/ | | |
| | | L1377D/V1439E | | |
| 1599/1600 | Y1281Q | V63Q/L171P/H212A/S260T/E306G/ | | + |
| | | M320R/S332P/F387I/I390A/V393G/ | | |
| | | V534I/M558W/M681G/K732W/L749F/ | | |
| | | L750D/I775G/T1148N/L1248D/Y1281Q/ | | |
| | | L1377D/V1439E | | |
| 1601/1602 | Y1281P | V63Q/L171P/H212A/S260T/E306G/ | + | + |
| | | M320R/S332P/F387I/I390A/V393G/ | | |
| | | V534I/M558W/M681G/K732W/L749F/ | | |
| | | L750D/I775G/T1148N/L1248D/Y1281P/ | | |
| | | L1377D/V1439E | | |
| 1603/1604 | S916E | V63Q/L171P/H212A/S260T/E306G/ | + | + |
| | | M320R/S332P/F387I/I390A/V393G/ | | |
| | | V534I/M558W/M681G/K732W/L749F/ | | |
| | | L750D/I775G/S916E/T1148N/L1248D/ | | |
| | | L1377D/V1439E | | |
| 1605/1606 | M1051W | V63Q/L171P/H212A/S260T/E306G/ | ++ | ++ |
| | | M320R/S332P/F387I/I390A/V393G/ | | |
| | | V534I/M558W/M681G/K732W/L749F/ | | |
| | | L750D/I775G/M1051W/T1148N/L1248D/ | | |
| | | L1377D/V1439E | | |
| 1607/1608 | L187C | V63Q/L171P/L187C/H212A/S260T/ | + | + |
| | | E306G/M320R/S332P/F387I/I390A/ | | |
| | | V393G/V534I/M558W/M681G/K732W/ | | |
| | | 9L74F/L750D/I775G/T1148N/L1248D/ | | |
| | | L1377D/V1439E | | |
| 1609/1610 | I215E | V63Q/L171P/H212A/I215E/S260T/ | | + |
| | | E306G/M320R/S332P/F387I/I390A/ | | |
| | | V393G/V534I/M558W/M681G/K732W/ | | |
| | | L749F/L750D/I775G/T1148N/L1248D/ | | |
| | | L1377D/V1439E | | |
| 1611/1612 | I215P | V63Q/L171P/H212A/I215P/S260T/ | + | + |
| | | E306G/M320R/S332P/F387I/I390A/ | | |
| | | V393G/V534I/M558W/M681G/K732W/ | | |
| | | L749F/L750D/I775G/T1148N/L1248D/ | | |
| | | L1377D/V1439E | | |
| 1613/1614 | I215S | V63Q/L171P/H212A/I215S/S260T/ | | + |
| | | E306G/M320R/S332P/F387I/I390A/ | | |
| | | V393G/V534I/M558W/M681G/K732W/ | | |
| | | L749F/L750D/I775G/T1148N/L1248D/ | | |
| | | L1377D/V1439E | | |
| 1615/1616 | L178C | V63Q/L171P/L178C/H212A/S260T/ | ++ | ++ |
| | | E306G/M320R/S332P/F387I/I390A/ | | |
| | | V393G/V534I/M558W/M681G/K732W/ | | |
| | | L749F/L750D/I775G/T1148N/L1248D/ | | |
| | | L1377D/V1439E | | |
| 1617/1618 | I1050T | V63Q/L171P/H212A/S260T/E306G/ | ++ | + |
| | | 320R/S332P/F387I/I390A/V393G/ | | |
| | | V534I/M558W/M681G/K732W/L749F/ | | |
| | | L750D/I775G/I1050T/T1148N/L1248D/ | | |
| | | L1377D/V1439E | | |
| 1619/1620 | K1210T | V63Q/L171P/H212A/S260T/E306G/ | ++ | + |
| | | 320R/S332P/F387I/I390A/V393G/ | | |
| | | V534I/M558W/M681G/K732W/L749F/ | | |
| | | L750D/I775G/T1148N/K1210T/L1248D/ | | |
| | | L1377D/V1439E | | |
| 1621/1622 | Y1281K | V63Q/L171P/H212A/S260T/E306G/ | ++ | + |
| | | 320R/S332P/F387I/I390A/V393G/ | | |
| | | V534I/M558W/M681G/K732W/L749F/ | | |
| | | L750D/I775G/T1148N/L1248D/Y1281K/ | | |
| | | L1377D/V1439E | | |
| 1623/1624 | R1042A | V63Q/L171P/H212A/S260T/E306G/ | + | + |
| | | 320R/S332P/F387I/I390A/V393G/ | | |
| | | V534I/M558W/M681G/K732W/L749F/ | | |
| | | L750D/I775G/R1042A/T1148N/L1248D/ | | |
| | | L1377D/V1439E | | |
| 1625/1626 | V104S | V63Q/V104S/L171P/H212A/S260T/ | + | + |
| | | E306G/M320R/S332P/F387I/I390A/ | | |
| | | V393G/V534I/M558W/M681G/K732W/ | | |
| | | L749F/L750D/I775G/T1148N/L1248D/ | | |
| | | L1377D/V1439E | | |
| 1627/1628 | P615D | V63Q/L171P/H212A/S260T/E306G/ | + | + |
| | | 320R/S332P/F387I/I390A/V393G/ | | |
| | | V534I/M558W/P615D/M681G/K732W/ | | |
| | | L749F/L750D/I775G/T1148N/L1248D/ | | |
| | | L1377D/V1439E | | |
| 1629/1630 | I610S | V63Q/L171P/H212A/S260T/E306G/ | + | ++ |
| | | 320R/S332P/F387I/I390A/V393G/ | | |
| | | V534I/M558W/I610S/M681G/K732W/ | | |
| | | L749F/L750D/I775G/T1148N/L1248D/ | | |
| | | L1377D/V1439E | | |
| 1631/1632 | I610K | V63Q/L171P/H212A/S260T/E306G/ | | + |
| | | 320R/S332P/F387I/I390A/V393G/ | | |
| | | V534I/M558W/I610K/M681G/K732W/ | | |
| | | L749F/L750D/I775G/T1148N/L1248D/ | | |
| | | L1377D/V1439E | | |
| 1633/1634 | I610T | V63Q/L171P/H212A/S260T/E306G/ | + | ++ |
| | | 320R/S332P/F387I/I390A/V393G/ | | |
| | | V534I/M558W/I610T/M681G/K732W/ | | |
| | | L749F/L750D/I775G/T1148N/L1248D/ | | |
| | | L1377D/V1439E | | |
| 1635/1636 | V536P | V63Q/L171P/H212A/S260T/E306G/ | + | ++ |
| | | 320R/S332P/F387I/I390A/V393G/ | | |
| | | V534I/V536P/M558W/M681G/K732W/ | | |
| | | L749F/L750D/I775G/T1148N/L1248D/ | | |
| | | L1377D/V1439E | | |
| 1637/1638 | A618Y | V63Q/L171P/H212A/S260T/E306G/ | ++ | + |
| | | 320R/S332P/F387I/I390A/V393G/ | | |
| | | V534I/M558W/A618Y/M681G/K732W/ | | |
| | | L749F/L750D/I775G/T1148N/L1248D/ | | |
| | | L1377D/V1439E | | |
| 1639/1640 | Q621W | V63Q/L171P/H212A/S260T/E306G/ | + | + |
| | | 320R/S332P/F387I/I390A/V393G/ | | |
| | | V534I/M558W/Q621W/M681G/K732W/ | | |
| | | L749F/L750D/I775G/T1148N/L1248D/ | | |
| | | L1377D/V1439E | | |
| 1641/1642 | A618P | V63Q/L171P/H212A/S260T/E306G/ | ++ | ++ |
| | | 320R/S332P/F387I/I390A/V393G/ | | |
| | | V534I/M558W/A618P/M681G/K732W/ | | |
| | | L749F/L750D/I775G/T1148N/L1248D/ | | |
| | | L1377D/V1439E | | |
| 1643/1644 | L614F | V63Q/L171P/H212A/S260T/E306G/ | | + |
| | | 320R/S332P/F387I/I390A/V393G/ | | |
| | | V534I/M558W/L614F/M681G/K732W/ | | |
| | | L749F/L750D/I775G/T1148N/L1248D/ | | |
| | | L1377D/V1439E | | |
| 1645/1646 | M110H | V63Q/M110H/L171P/H212A/S260T/ | | + |
| | | E306G/M320R/S332P/F387I/I390A/ | | |
| | | V393G/V534I/M558W/M681G/K732W/ | | |
| | | L749F/L750D/I775G/T1148N/L1248D/ | | |
| | | L1377D/V1439E | | |
| 1647/1648 | R301K | V63Q/L171P/H212A/S260T/R301K/ | | + |
| | | E306G/M320R/S332P/F387I/I390A/ | | |
| | | V393G/V534I/M558W/M681G/K732W/ | | |
| | | L749F/L750D/I775G/T1148N/L1248D/ | | |
| | | L1377D/V1439E | | |

All activities were determined relative to the reference polypoptide of SEQ ID NO: 1514. Levels of increased activity are defined as follows: “+” 0.9 to 1.1, “++” >1.1

TABLE 10.2
Activity of FVIII Variants Relative to SEQ ID NO: 1514

| | | | | | | | | | (HepG2) | |
|---|---|---|---|---|---|---|---|---|---|---|
| | | | | | (Expi293F) | (Expi293F) | | | Chromo | (HepG2) |
| | | | | | Chromogenic | Clotting | | | genic | Clotting |
| | Amino Acid | | (Expi293F) | (Expi293F) | Activity | Activity | (HepG2) | (HepG2) | Activity | Activity |
| | Differences | Amino Acid | Unchallenged | Unchallenged | after | after | Unchallenged | Unchallenged | after | after |
| SEQ ID | (Relative to | Differences | Chromogenic | Clotting | Plasma | Plasma | Chromogenic | Clotting | Plasma | Plasma |
| NO: | SEQ ID | (Relative to | Activity | Activity | Challenge | Challenge | Activity | Activity | Challenge | Challenge |
| (nt/aa) | NO: 1514) | SEQ ID NO: 2) | FIOPC | FIOPC | FIOPC | FIOPC | FIOPC | FIOPC | FIOPC | FIOPC |
| 1513/1514 | | V63Q/L171P/H212A/S260T/E306G/ | + | + | + | + | + | + | + | + |
| | | M320R/S332P/F387I/I390A/V393G/ | | | | | | | | |
| | | V534I/M558W/M681G/K732W/ | | | | | | | | |
| | | L749F/L750D/I775G/T1148N/ | | | | | | | | |
| | | L1248D/L1377D/V1439E | | | | | | | | |
| 1649/1650 | I1078S | V63Q/L171P/H212A/S260T/E306G/ | ++ | + | ++ | ++ | + | + | | |
| | | M320R/S332P/F387I/I390A/V393G/ | | | | | | | | |
| | | V534I/M558W/M681G/K732W/ | | | | | | | | |
| | | L749F/L750D/I775G/I1078S/ | | | | | | | | |
| | | T1148N/L1248D/L1377D/V1439E | | | | | | | | |
| 1651/1652 | S812G | V63Q/L171P/H212A/S260T/E306G/ | ++ | + | ++ | ++ | | + | | |
| | | M320R/S332P/F387I/I390A/V393G/ | | | | | | | | |
| | | V534I/M558W/M681G/K732W/ | | | | | | | | |
| | | L749F/L750D/I775G/S812G/ | | | | | | | | |
| | | T1148N/L1248D/L1377D/V1439E | | | | | | | | |
| 1653/1654 | E786C | V63Q/L171P/H212A/S260T/E306G/ | ++ | + | + | | ++ | ++ | | |
| | | M320R/S332P/F387I/I390A/V393G/ | | | | | | | | |
| | | V534I/M558W/M681G/K732W/ | | | | | | | | |
| | | L749F/L750D/I775G/E786C/ | | | | | | | | |
| | | T1148N/L1248D/L1377D/V1439E | | | | | | | | |
| 1655/1656 | A127V | V63Q/A127V/L171P/H212A/S260T/ | + | + | ++ | ++ | + | + | + | + |
| | | E306G/M320R/S332P/F387I/I390A/ | | | | | | | | |
| | | V393G/V534I/M558W/M681G/ | | | | | | | | |
| | | K732W/L749F/L750D/I775G/ | | | | | | | | |
| | | T1148N/L1248D/L1377D/V1439E | | | | | | | | |
| 1657/1658 | Y175G | V63Q/L171P/Y175G/H212A/S260T/ | | | | | | + | | |
| | | E306G/M320R/S332P/F387I/I390A/ | | | | | | | | |
| | | V393G/V534I/M558W/M681G/ | | | | | | | | |
| | | K732W/L749F/L750D/I775G/ | | | | | | | | |
| | | T1148N/L1248D/L1377D/V1439E | | | | | | | | |
| 1659/1660 | K1093W | V63Q/L171P/H212A/S260T/E306G/ | ++ | ++ | ++ | ++ | + | ++ | + | ++ |
| | | M320R/S332P/F387I/I390A/V393G/ | | | | | | | | |
| | | V534I/M558W/M681G/K732W/ | | | | | | | | |
| | | L749F/L750D/I775G/K1093W/ | | | | | | | | |
| | | T1148N/L1248D/L1377D/V1439E | | | | | | | | |
| 1661/1662 | F1088G | V63Q/L171P/H212A/S260T/E306G/ | ++ | + | ++ | ++ | | ++ | | + |
| | | M320R/S332P/F387I/I390A/V393G/ | | | | | | | | |
| | | V534I/M558W/M681G/K732W/ | | | | | | | | |
| | | L749F/L750D/I775G/F1088G/T1148N/ | | | | | | | | |
| | | L1248D/L1377D/V1439E | | | | | | | | |
| 1663/1664 | K1093T | V63Q/L171P/H212A/S260T/E306G/ | ++ | ++ | + | ++ | ++ | ++ | + | + |
| | | M320R/S332P/F387I/I390A/V393G/ | | | | | | | | |
| | | V534I/M558W/M681G/K732W/ | | | | | | | | |
| | | L749F/L750D/I775G/K1093T/T1148N/ | | | | | | | | |
| | | L1248D/L1377D/V1439E | | | | | | | | |
| 1665/1666 | L650M | V63Q/L171P/H212A/S260T/E306G/ | | + | + | ++ | ++ | ++ | ++ | |
| | | M320R/S332P/F387I/I390A/V393G/ | | | | | | | | |
| | | V534I/M558W/L650M/M681G/ | | | | | | | | |
| | | K732W/L749F/L750D/I775G/T1148N/ | | | | | | | | |
| | | L1248D/L1377D/V1439E | | | | | | | | |
| 1667/1668 | R301M | V63Q/L171P/H212A/S260T/R301M/ | + | + | + | | | | | + |
| | | E306G/M320R/S332P/F387I/I390A/ | | | | | | | | |
| | | V393G/V534I/M558W/M681G/ | | | | | | | | |
| | | K732W/L749F/L750D/I775G/T1148N/ | | | | | | | | |
| | | L1248D/L1377D/V1439E | | | | | | | | |
| 1669/1670 | T535N | V63Q/L171P/H212A/S260T/E306G/ | ++ | + | ++ | ++ | | + | | |
| | | M320R/S332P/F387I/I390A/V393G/ | | | | | | | | |
| | | V534I/T535N/M558W/M681G/ | | | | | | | | |
| | | K732W/L749F/L750D/I775G/T1148N/ | | | | | | | | |
| | | L1248D/L1377D/V1439E | | | | | | | | |
| 1671/1672 | D806R | V63Q/L171P/H212A/S260T/E306G/ | ++ | + | ++ | ++ | ++ | ++ | + | |
| | | M320R/S332P/F387I/I390A/V393G/ | | | | | | | | |
| | | V534I/M558W/M681G/K732W/ | | | | | | | | |
| | | L749F/L750D/I775G/D806R/T1148N/ | | | | | | | | |
| | | L1248D/L1377D/V1439E | | | | | | | | |
| 1673/1674 | Y738E | V63Q/L171P/H212A/S260T/E306G/ | ++ | + | + | + | ++ | ++ | ++ | |
| | | M320R/S332P/F387I/I390A/V393G/ | | | | | | | | |
| | | V534I/M558W/M681G/K732W/ | | | | | | | | |
| | | Y738E/L749F/L750D/I775G/T1148N/ | | | | | | | | |
| | | L1248D/L1377D/V1439E | | | | | | | | |
| 1675/1676 | S700E | V63Q/L171P/H212A/S260T/E306G/ | ++ | ++ | + | ++ | ++ | ++ | ++ | |
| | | M320R/S332P/F387I/I390A/V393G/ | | | | | | | | |
| | | V534I/M558W/M681G/S700E/ | | | | | | | | |
| | | K732W/L749F/L750D/I775G/T1148N/ | | | | | | | | |
| | | L1248D/L1377D/V1439E | | | | | | | | |
| 1677/1678 | E132R | V63Q/E132R/L171P/H212A/S260T/ | | | + | + | ++ | + | ++ | |
| | | E306G/M320R/S332P/F387I/I390A/ | | | | | | | | |
| | | V393G/V534I/M558W/M681G/ | | | | | | | | |
| | | K732W/L749F/L750D/I775G/T1148N/ | | | | | | | | |
| | | L1248D/L1377D/V1439E | | | | | | | | |
| 1679/1680 | E1076G | V63Q/L171P/H212A/S260T/E306G/ | + | + | ++ | ++ | ++ | + | | + |
| | | M320R/S332P/F387I/I390A/V393G/ | | | | | | | | |
| | | V534I/M558W/M681G/K732W/ | | | | | | | | |
| | | L749F/L750D/I775G/E1076G/T1148N/ | | | | | | | | |
| | | L1248D/L1377D/V1439E | | | | | | | | |
| 1681/1682 | S1084L | V63Q/L171P/H212A/S260T/E306G/ | | + | ++ | | | | | + |
| | | M320R/S332P/F387I/I390A/V393G/ | | | | | | | | |
| | | V534I/M558W/M681G/K732W/ | | | | | | | | |
| | | L749F/L750D/I775G/S1084L/T1148N/ | | | | | | | | |
| | | L1248D/L1377D/V1439E | | | | | | | | |
| 1683/1684 | G498A | V63Q/L171P/H212A/S260T/E306G/ | + | + | ++ | | | + | | |
| | | M320R/S332P/F387I/I390A/V393G/ | | | | | | | | |
| | | G498A/V534I/M558W/M681G/ | | | | | | | | |
| | | K732W/L749F/L750D/I775G/T1148N/ | | | | | | | | |
| | | L1248D/L1377D/V1439E | | | | | | | | |
| 1685/1686 | E786L | V63Q/L171P/H212A/S260T/E306G/ | + | + | ++ | ++ | | + | + | + |
| | | M320R/S332P/F387I/I390A/V393G/ | | | | | | | | |
| | | V534I/M558W/M681G/K732W/ | | | | | | | | |
| | | L749F/L750D/I775G/E786L/T1148N/ | | | | | | | | |
| | | L1248D/L1377D/V1439E | | | | | | | | |
| 1687/1688 | Y738F | V63Q/L171P/H212A/S260T/E306G/ | + | | + | + | ++ | | + | |
| | | M320R/S332P/F387I/I390A/V393G/ | | | | | | | | |
| | | V534I/M558W/M681G/K732W/ | | | | | | | | |
| | | Y738F/L749F/L750D/I775G/T1148N/ | | | | | | | | |
| | | L1248D/L1377D/V1439E | | | | | | | | |
| 1689/1690 | T535G | V63Q/L171P/H212A/S260T/E306G/ | ++ | ++ | + | ++ | ++ | + | | ++ |
| | | M320R/S332P/F387I/I390A/V393G/ | | | | | | | | |
| | | V534I/T535G/M558W/M681G/ | | | | | | | | |
| | | K732W/L749F/L750D/I775G/T1148N/ | | | | | | | | |
| | | L1248D/L1377D/V1439E | | | | | | | | |
| 1691/1692 | D806E | V63Q/L171P/H212A/S260T/E306G/ | + | + | + | | + | | | + |
| | | M320R/S332P/F387I/I390A/V393G/ | | | | | | | | |
| | | V534I/M558W/M681G/K732W/ | | | | | | | | |
| | | L749F/L750D/I775G/D806E/T1148N/ | | | | | | | | |
| | | L1248D/L1377D/V1439E | | | | | | | | |
| 1693/1694 | I756G | V63Q/L171P/H212A/S260T/E306G/ | ++ | + | ++ | | ++ | + | + | |
| | | M320R/S332P/F387I/I390A/V393G/ | | | | | | | | |
| | | V534I/M558W/M681G/K732W/ | | | | | | | | |
| | | L749F/L750D/I756G/I775G/T1148N/ | | | | | | | | |
| | | L1248D/L1377D/V1439E | | | | | | | | |
| 1695/1696 | F1108S | V63Q/L171P/H212A/S260T/E306G/ | ++ | + | ++ | ++ | ++ | + | + | + |
| | | M320R/S332P/F387I/I390A/V393G/ | | | | | | | | |
| | | V534I/M558W/M681G/K732W/ | | | | | | | | |
| | | L749F/L750D/I775G/F1108S/T1148N/ | | | | | | | | |
| | | L1248D/L1377D/V1439E | | | | | | | | |
| 1697/1698 | K1093V | V63Q/L171P/H212A/S260T/E306G/ | ++ | ++ | + | ++ | | ++ | | + |
| | | M320R/S332P/F387I/I390A/V393G/ | | | | | | | | |
| | | V534I/M558W/M681G/K732W/ | | | | | | | | |
| | | L749F/L750D/I775G/K1093V/ | | | | | | | | |
| | | T1148N/L1248D/L1377D/V1439E | | | | | | | | |
| 1699/1700 | S746P | V63Q/L171P/H212A/S260T/E306G/ | ++ | | + | + | | | | |
| | | M320R/S332P/F387I/I390A/V393G/ | | | | | | | | |
| | | V534I/M558W/M681G/K732W/ | | | | | | | | |
| | | S746P/L749F/L750D/I775G/T1148N/ | | | | | | | | |
| | | L1248D/L1377D/V1439E | | | | | | | | |
| 1701/1702 | L160E | V63Q/L160E/L171P/H212A/S260T/ | ++ | + | + | + | + | + | + | + |
| | | E306G/M320R/S332P/F387I/I390A/ | | | | | | | | |
| | | V393G/V534I/M558W/M681G/ | | | | | | | | |
| | | K732W/L749F/L750D/I775G/T1148N/ | | | | | | | | |
| | | L1248D/L1377D/V1439E | | | | | | | | |
| 1703/1704 | L160T | V63Q/L160T/L171P/H212A/S260T/ | + | + | + | + | + | + | + | |
| | | E306G/M320R/S332P/F387I/I390A/ | | | | | | | | |
| | | V393G/V534I/M558W/M681G/ | | | | | | | | |
| | | K732W/L749F/L750D/I775G/T1148N/ | | | | | | | | |
| | | L1248D/L1377D/V1439E | | | | | | | | |
| 1705/1706 | L178V | V63Q/L171P/L178V/H212A/S260T/ | + | + | + | ++ | +++ | + | + | + |
| | | E306G/M320R/S332P/F387I/I390A/ | | | | | | | | |
| | | V393G/V534I/M558W/M681G/ | | | | | | | | |
| | | K732W/L749F/L750D/I775G/T1148N/ | | | | | | | | |
| | | L1248D/L1377D/V1439E | | | | | | | | |
| 1707/1708 | S543L | V63Q/L171P/H212A/S260T/E306G/ | + | + | ++ | | +++ | | + | + |
| | | M320R/S332P/F387I/I390A/V393G/ | | | | | | | | |
| | | V534I/S543L/M558W/M681G/ | | | | | | | | |
| | | K732W/L749F/L750D/I775G/T1148N/ | | | | | | | | |
| | | L1248D/L1377D/V1439E | | | | | | | | |
| 1709/1710 | I310G | V63Q/L171P/H212A/S260T/E306G/ | ++ | ++ | ++ | ++ | | + | | |
| | | I310G/M320R/S332P/F387I/I390A/ | | | | | | | | |
| | | V393G/V534I/M558W/M681G/ | | | | | | | | |
| | | K732W/L749F/L750D/I775G/T1148N/ | | | | | | | | |
| | | L1248D/L1377D/V1439E | | | | | | | | |
| 1711/1712 | S700Q | V63Q/L171P/H212A/S260T/E306G/ | ++ | + | ++ | + | ++ | ++ | ++ | |
| | | M320R/S332P/F387I/I390A/V393G/ | | | | | | | | |
| | | V534I/M558W/M681G/S700Q/ | | | | | | | | |
| | | K732W/L749F/L750D/I775G/T1148N/ | | | | | | | | |
| | | L1248D/L1377D/V1439E | | | | | | | | |
| 1713/1714 | K1093A | V63Q/L171P/H212A/S260T/E306G/ | ++ | ++ | +++ | ++ | + | ++ | ++ | |
| | | M320R/S332P/F387I/I390A/V393G/ | | | | | | | | |
| | | V534I/M558W/M681G/K732W/ | | | | | | | | |
| | | L749F/L750D/I775G/K1093A/ | | | | | | | | |
| | | T1148N/L1248D/L1377D/V1439E | | | | | | | | |
| 1715/1716 | Q584A | V63Q/L171P/H212A/S260T/E306G/ | | + | | + | | | | |
| | | M320R/S332P/F387I/I390A/V393G/ | | | | | | | | |
| | | V534I/M558W/Q584A/M681G/ | | | | | | | | |
| | | K732W/L749F/L750D/I775G/T1148N/ | | | | | | | | |
| | | L1248D/L1377D/V1439E | | | | | | | | |
| 1717/1718 | E918S | V63Q/L171P/H212A/S260T/E306G/ | ++ | ++ | +++ | | ++ | ++ | ++ | |
| | | M320R/S332P/F387I/I390A/V393G/ | | | | | | | | |
| | | V534I/M558W/M681G/K732W/ | | | | | | | | |
| | | L749F/L750D/I775G/E918S/T1148N/ | | | | | | | | |
| | | L1248D/L1377D/V1439E | | | | | | | | |
| 1719/1720 | E1452L | V63Q/L171P/H212A/S260T/E306G/ | + | + | + | + | + | + | + | |
| | | M320R/S332P/F387I/I390A/V393G/ | | | | | | | | |
| | | V534I/M558W/M681G/K732W/ | | | | | | | | |
| | | L749F/L750D/I775G/T1148N/L1248D/ | | | | | | | | |
| | | L1377D/V1439E/E1452L | | | | | | | | |
| 1721/1722 | Y738P | V63Q/L171P/H212A/S260T/E306G/ | | | + | | | + | | |
| | | M320R/S332P/F387I/I390A/V393G/ | | | | | | | | |
| | | V534I/M558W/M681G/K732W/ | | | | | | | | |
| | | Y738P/L749F/L750D/I775G/T1148N/ | | | | | | | | |
| | | L1248D/L1377D/V1439E | | | | | | | | |
| 1723/1724 | D790W | V63Q/L171P/H212A/S260T/E306G/ | + | + | ++ | | ++ | + | | |
| | | M320R/S332P/F387I/I390A/V393G/ | | | | | | | | |
| | | V534I/M558W/M681G/K732W/ | | | | | | | | |
| | | L749F/L750D/I775G/D790W/T1148N/ | | | | | | | | |
| | | L1248D/L1377D/V1439E | | | | | | | | |
| 1725/1726 | D790I | V63Q/L171P/H212A/S260T/E306G/ | ++ | + | ++ | + | + | + | | |
| | | M320R/S332P/F387I/I390A/V393G/ | | | | | | | | |
| | | V534I/M558W/M681G/K732W/ | | | | | | | | |
| | | L749F/L750D/I775G/D790I/T1148N/ | | | | | | | | |
| | | L1248D/L1377D/V1439E | | | | | | | | |
| 1727/1728 | G292D | V63Q/L171P/H212A/S260T/G292D/ | + | + | ++ | | ++ | + | + | |
| | | E306G/M320R/S332P/F387I/I390A/ | | | | | | | | |
| | | V393G/V534I/M558W/M681G/ | | | | | | | | |
| | | K732W/L749F/L750D/I775G/T1148N/ | | | | | | | | |
| | | L1248D/L1377D/V1439E | | | | | | | | |
| 1729/1730 | E800M | V63Q/L171P/H212A/S260T/E306G/ | + | + | ++ | | + | + | + | |
| | | M320R/S332P/F387I/I390A/V393G/ | | | | | | | | |
| | | V534I/M558W/M681G/K732W/ | | | | | | | | |
| | | L749F/L750D/I775G/E800M/T1148N/ | | | | | | | | |
| | | L1248D/L1377D/V1439E | | | | | | | | |
| 1731/1732 | F1108M | V63Q/L171P/H212A/S260T/E306G/ | + | + | ++ | ++ | ++ | + | ++ | + |
| | | M320R/S332P/F387I/I390A/V393G/ | | | | | | | | |
| | | V534I/M558W/M681G/K732W/ | | | | | | | | |
| | | L749F/L750D/I775G/F1108M/ | | | | | | | | |
| | | T1148N/L1248D/L1377D/V1439E | | | | | | | | |
| 1733/1734 | F1088S | V63Q/L171P/H212A/S260T/E306G/ | | + | | + | | + | | |
| | | M320R/S332P/F387I/I390A/V393G/ | | | | | | | | |
| | | V534I/M558W/M681G/K732W/ | | | | | | | | |
| | | L749F/L750D/I775G/F1088S/T1148N/ | | | | | | | | |
| | | L1248D/L1377D/V1439E | | | | | | | | |
| 1735/1736 | A167G | V63Q/A167G/L171P/H212A/S260T/ | + | + | + | + | ++ | + | + | + |
| | | E306G/M320R/S332P/F387I/I390A/ | | | | | | | | |
| | | V393G/V534I/M558W/M681G/ | | | | | | | | |
| | | K732W/L749F/L750D/I775G/T1148N/ | | | | | | | | |
| | | L1248D/L1377D/V1439E | | | | | | | | |
| 1737/1738 | V892T | V63Q/L171P/H212A/S260T/E306G/ | | + | + | + | | + | + | + |
| | | M320R/S332P/F387I/I390A/V393G/ | | | | | | | | |
| | | V534I/M558W/M681G/K732W/ | | | | | | | | |
| | | L749F/L750D/I775G/V892T/T1148N/ | | | | | | | | |
| | | L1248D/L1377D/V1439E | | | | | | | | |
| 1739/1740 | D790P | V63Q/L171P/H212A/S260T/E306G/ | ++ | + | ++ | | | + | + | |
| | | M320R/S332P/F387I/I390A/V393G/ | | | | | | | | |
| | | V534I/M558W/M681G/K732W/ | | | | | | | | |
| | | L749F/L750D/I775G/D790P/T1148N/ | | | | | | | | |
| | | L1248D/L1377D/V1439E | | | | | | | | |
| 1741/1742 | E800R | V63Q/L171P/H212A/S260T/E306G/ | + | + | ++ | + | | + | | + |
| | | M320R/S332P/F387I/I390A/V393G/ | | | | | | | | |
| | | V534I/M558W/M681G/K732W/ | | | | | | | | |
| | | L749F/L750D/I775G/E800R/T1148N/ | | | | | | | | |
| | | L1248D/L1377D/V1439E | | | | | | | | |
| 1743/1744 | Y738K | V63Q/L171P/H212A/S260T/E306G/ | ++ | | + | + | +++ | + | + | + |
| | | M320R/S332P/F387I/I390A/V393G/ | | | | | | | | |
| | | V534I/M558W/M681G/K732W/ | | | | | | | | |
| | | Y738K/L749F/L750D/I775G/T1148N/ | | | | | | | | |
| | | L1248D/L1377D/V1439E | | | | | | | | |
| 1745/1746 | S304G | V63Q/L171P/H212A/S260T/S304G/ | + | + | + | ++ | | | | |
| | | E306G/M320R/S332P/F387I/I390A/ | | | | | | | | |
| | | V393G/V534I/M558W/M681G/ | | | | | | | | |
| | | K732W/L749F/L750D/I775G/T1148N/ | | | | | | | | |
| | | L1248D/L1377D/V1439E | | | | | | | | |
| 1747/1748 | S1116G | V63Q/L171P/H212A/S260T/E306G/ | + | + | + | ++ | ++ | + | | |
| | | M320R/S332P/F387I/I390A/V393G/ | | | | | | | | |
| | | V534I/M558W/M681G/K732W/ | | | | | | | | |
| | | L749F/L750D/I775G/S1116G/T1148N/ | | | | | | | | |
| | | L1248D/L1377D/V1439E | | | | | | | | |
| 1749/1750 | L178Q | V63Q/L171P/L178Q/H212A/S260T/ | ++ | + | + | | ++++ | ++ | ++ | ++ |
| | | E306G/M320R/S332P/F387I/I390A/ | | | | | | | | |
| | | V393G/V534I/M558W/M681G/ | | | | | | | | |
| | | K732W/L749F/L750D/I775G/T1148N/ | | | | | | | | |
| | | L1248D/L1377D/V1439E | | | | | | | | |
| 1751/1752 | S1116A | V63Q/L171P/H212A/S260T/E306G/ | + | + | + | + | | + | | |
| | | M320R/S332P/F387I/I390A/V393G/ | | | | | | | | |
| | | V534I/M558W/M681G/K732W/ | | | | | | | | |
| | | L749F/L750D/I775G/S1116A/T1148N/ | | | | | | | | |
| | | L1248D/L1377D/V1439E | | | | | | | | |
| 1753/1754 | K752G | V63Q/L171P/H212A/S260T/E306G/ | + | ++ | ++ | | ++ | ++ | + | + |
| | | M320R/S332P/F387I/I390A/V393G/ | | | | | | | | |
| | | V534I/M558W/M681G/K732W/ | | | | | | | | |
| | | L749F/L750D/K752G/I775G/T1148N/ | | | | | | | | |
| | | L1248D/L1377D/V1439E | | | | | | | | |
| 1755/1756 | F1108W | V63Q/L171P/H212A/S260T/E306G/ | + | + | | + | ++ | + | | |
| | | M320R/S332P/F387I/I390A/V393G/ | | | | | | | | |
| | | V534I/M558W/M681G/K732W/ | | | | | | | | |
| | | L749F/L750D/I775G/F1108W/T1148N/ | | | | | | | | |
| | | L1248D/L1377D/V1439E | | | | | | | | |
| 1757/1758 | Y738D | V63Q/L171P/H212A/S260T/E306G/ | ++ | + | ++ | | + | + | + | ++ |
| | | M320R/S332P/F387I/I390A/V393G/ | | | | | | | | |
| | | V534I/M558W/M681G/K732W/ | | | | | | | | |
| | | Y738D/L749F/L750D/I775G/T1148N/ | | | | | | | | |
| | | L1248D/L1377D/V1439E | | | | | | | | |
| 1759/1760 | T281A | V63Q/L171P/H212A/S260T/T281A/ | | + | | ++ | + | + | + | + |
| | | E306G/M320R/S332P/F387I/I390A/ | | | | | | | | |
| | | V393G/V534I/M558W/M681G/ | | | | | | | | |
| | | K732W/L749F/L750D/I775G/T1148N/ | | | | | | | | |
| | | L1248D/L1377D/V1439E | | | | | | | | |
| 1761/1762 | Y917G | V63Q/L171P/H212A/S260T/E306G/ | + | + | ++ | + | ++ | + | + | + |
| | | M320R/S332P/F387I/I390A/V393G/ | | | | | | | | |
| | | V534I/M558W/M681G/K732W/ | | | | | | | | |
| | | L749F/L750D/I775G/Y917G/T1148N/ | | | | | | | | |
| | | L1248D/L1377D/V1439E | | | | | | | | |
| 1763/1764 | H712M | V63Q/L171P/H212A/S260T/E306G/ | + | + | + | ++ | ++ | + | ++ | |
| | | M320R/S332P/F387I/I390A/V393G/ | | | | | | | | |
| | | V534I/M558W/M681G/H712M/ | | | | | | | | |
| | | K732W/L749F/L750D/I775G/T1148N/ | | | | | | | | |
| | | L1248D/L1377D/V1439E | | | | | | | | |
| 1765/1766 | S1080V | V63Q/L171P/H212A/S260T/E306G/ | ++ | + | + | ++ | + | + | | + |
| | | M320R/S332P/F387I/I390A/V393G/ | | | | | | | | |
| | | V534I/M558W/M681G/K732W/ | | | | | | | | |
| | | L749F/L750D/I775G/S1080V/T1148N/ | | | | | | | | |
| | | L1248D/L1377D/V1439E | | | | | | | | |
| 1767/1768 | D806G/ | V63Q/L171P/H212A/S260T/E306G/ | ++ | + | ++ | ++ | + | | | + |
| | K1352E | M320R/S332P/F387I/I390A/V393G/ | | | | | | | | |
| | | V534I/M558W/M681G/K732W/ | | | | | | | | |
| | | L749F/L750D/I775G/D806G/T1148N/ | | | | | | | | |
| | | L1248D/K1352E/L1377D/ | | | | | | | | |
| | | V1439E | | | | | | | | |
| 1769/1770 | E1095G | V63Q/L171P/H212A/S260T/E306G/ | + | + | | | + | ++ | | + |
| | | M320R/S332P/F387I/I390A/V393G/ | | | | | | | | |
| | | V534I/M558W/M681G/K732W/ | | | | | | | | |
| | | L749F/L750D/I775G/E1095G/T1148N/ | | | | | | | | |
| | | L1248D/L1377D/V1439E | | | | | | | | |
| 1771/1772 | K1093G | V63Q/L171P/H212A/S260T/E306G/ | ++ | ++ | ++ | ++ | ++ | ++ | ++ | ++ |
| | | M320R/S332P/F387I/I390A/V393G/ | | | | | | | | |
| | | V534I/M558W/M681G/K732W/ | | | | | | | | |
| | | L749F/L750D/I775G/K1093G/ | | | | | | | | |
| | | T1148N/L1248D/L1377D/V1439E | | | | | | | | |
| 1773/1774 | D744A | V63Q/L171P/H212A/S260T/E306G/ | ++ | | + | | | | | |
| | | M320R/S332P/F387I/I390A/V393G/ | | | | | | | | |
| | | V534I/M558W/M681G/K732W/ | | | | | | | | |
| | | D744A/L749F/L750D/I775G/T1148N/ | | | | | | | | |
| | | L1248D/L1377D/V1439E | | | | | | | | |
| 1775/1776 | S543G | V63Q/L171P/H212A/S260T/E306G/ | ++ | + | + | ++ | ++ | ++ | | |
| | | M320R/S332P/F387I/I390A/V393G/ | | | | | | | | |
| | | V534I/S543G/M558W/M681G/ | | | | | | | | |
| | | K732W/L749F/L750D/I775G/T1148N/ | | | | | | | | |
| | | L1248D/L1377D/V1439E | | | | | | | | |
| 1777/1778 | M1098G | V63Q/L171P/H212A/S260T/E306G/ | + | + | | | + | + | + | + |
| | | M320R/S332P/F387I/I390A/V393G/ | | | | | | | | |
| | | V534I/M558W/M681G/K732W/ | | | | | | | | |
| | | L749F/L750D/I775G/M1098G/ | | | | | | | | |
| | | T1148N/L1248D/L1377D/V1439E | | | | | | | | |
| 1779/1780 | T281S | V63Q/L171P/H212A/S260T/T281S/E306G/ | ++ | + | + | + | + | + | | |
| | | M320R/S332P/F387I/I390A/ | | | | | | | | |
| | | V393G/V534I/M558W/M681G/K732W/ | | | | | | | | |
| | | L749F/L750D/I775G/T1148N/ | | | | | | | | |
| | | L1248D/L1377D/V1439E | | | | | | | | |
| 1781/1782 | I756T | V63Q/L171P/H212A/S260T/E306G/ | ++ | + | ++ | | ++ | + | + | ++ |
| | | M320R/S332P/F387I/I390A/V393G/ | | | | | | | | |
| | | V534I/M558W/M681G/K732W/ | | | | | | | | |
| | | L749F/L750D/I756T/I775G/T1148N/ | | | | | | | | |
| | | L1248D/L1377D/V1439E | | | | | | | | |
| 1783/1784 | M948S | V63Q/L171P/H212A/S260T/E306G/ | + | + | | | ++ | + | | + |
| | | M320R/S332P/F387I/I390A/V393G/ | | | | | | | | |
| | | V534I/M558W/M681G/K732W/ | | | | | | | | |
| | | L749F/L750D/I775G/M948S/T1148N/ | | | | | | | | |
| | | L1248D/L1377D/V1439E | | | | | | | | |
| 1785/1786 | A119V | V63Q/A119V/L171P/H212A/S260T/ | | + | | | | + | | |
| | | E306G/M320R/S332P/F387I/I390A/ | | | | | | | | |
| | | V393G/V534I/M558W/M681G/K732W/ | | | | | | | | |
| | | L749F/L750D/I775G/T1148N/ | | | | | | | | |
| | | L1248D/L1377D/V1439E | | | | | | | | |
| 1787/1788 | S1116L | V63Q/L171P/H212A/S260T/E306G/ | ++ | + | + | + | + | + | | + |
| | | M320R/S332P/F387I/I390A/V393G/ | | | | | | | | |
| | | V534I/M558W/M681G/K732W/ | | | | | | | | |
| | | L749F/L750D/I775G/S1116L/T1148N/ | | | | | | | | |
| | | L1248D/L1377D/V1439E | | | | | | | | |
| 1789/1790 | E785V | V63Q/L171P/H212A/S260T/E306G/ | ++ | + | + | + | + | + | + | + |
| | | M320R/S332P/F387I/I390A/V393G/ | | | | | | | | |
| | | V534I/M558W/M681G/K732W/ | | | | | | | | |
| | | L749F/L750D/I775G/E785V/T1148N/ | | | | | | | | |
| | | L1248D/L1377D/V1439E | | | | | | | | |
| 1791/1792 | M1113F | V63Q/L171P/H212A/S260T/E306G/ | + | + | + | | | + | | + |
| | | M320R/S332P/F387I/I390A/V393G/ | | | | | | | | |
| | | V534I/M558W/M681G/K732W/ | | | | | | | | |
| | | L749F/L750D/I775G/M1113F/T1148N/ | | | | | | | | |
| | | L1248D/L1377D/V1439E | | | | | | | | |
| 1793/1794 | S700A | V63Q/L171P/H212A/S260T/E306G/ | ++ | + | + | + | | + | | + |
| | | M320R/S332P/F387I/I390A/V393G/ | | | | | | | | |
| | | V534I/M558W/M681G/S700A/ | | | | | | | | |
| | | K732W/L749F/L750D/I775G/T1148N/ | | | | | | | | |
| | | L1248D/L1377D/V1439E | | | | | | | | |
| 1795/1796 | E786D | V63Q/L171P/H212A/S260T/E306G/ | + | + | + | ++ | | + | | + |
| | | M320R/S332P/F387I/I390A/V393G/ | | | | | | | | |
| | | V534I/M558W/M681G/K732W/ | | | | | | | | |
| | | L749F/L750D/I775G/E786D/T1148N/ | | | | | | | | |
| | | L1248D/L1377D/V1439E | | | | | | | | |
| 1797/1798 | Y175A | V63Q/L171P/Y175A/H212A/S260T/ | | + | | + | | | | + |
| | | E306G/M320R/S332P/F387I/I390A/ | | | | | | | | |
| | | V393G/V534I/M558W/M681G/K732W/ | | | | | | | | |
| | | L749F/L750D/I775G/T1148N/ | | | | | | | | |
| | | L1248D/L1377D/V1439E | | | | | | | | |
| 1799/1800 | D790M | V63Q/L171P/H212A/S260T/E306G/ | ++ | + | ++ | | + | + | + | + |
| | | M320R/S332P/F387I/I390A/V393G/ | | | | | | | | |
| | | V534I/M558W/M681G/K732W/ | | | | | | | | |
| | | L749F/L750D/I775G/D790M/T1148N/ | | | | | | | | |
| | | L1248D/L1377D/V1439E | | | | | | | | |
| 1801/1802 | F667I | V63Q/L171P/H212A/S260T/E306G/ | + | + | + | + | + | + | + | + |
| | | M320R/S332P/F387I/I390A/V393G/ | | | | | | | | |
| | | V534I/M558W/F667I/M681G/K732W/ | | | | | | | | |
| | | L749F/L750D/I775G/T1148N/ | | | | | | | | |
| | | L1248D/L1377D/V1439E | | | | | | | | |
| 1803/1804 | Q158G | V63Q/Q158G/L171P/H212A/S260T/ | + | + | | ++ | | + | | |
| | | E306G/M320R/S332P/F387I/I390A/ | | | | | | | | |
| | | V393G/V534I/M558W/M681G/K732W/ | | | | | | | | |
| | | L749F/L750D/I775G/T1148N/ | | | | | | | | |
| | | L1248D/L1377D/V1439E | | | | | | | | |
| 1805/1806 | Q580S | V63Q/L171P/H212A/S260T/E306G/ | ++ | + | + | ++ | + | + | + | + |
| | | M320R/S332P/F387I/I390A/V393G/ | | | | | | | | |
| | | V534I/M558W/Q580S/M681G/ | | | | | | | | |
| | | K732W/L749F/L750D/I775G/T1148N/ | | | | | | | | |
| | | L1248D/L1377D/V1439E | | | | | | | | |
| 1807/1808 | W707H | V63Q/L171P/H212A/S260T/E306G/ | ++ | + | + | | | + | | + |
| | | M320R/S332P/F387I/I390A/V393G/ | | | | | | | | |
| | | V534I/M558W/M681G/W707H/ | | | | | | | | |
| | | K732W/L749F/L750D/I775G/T1148N/ | | | | | | | | |
| | | L1248D/L1377D/V1439E | | | | | | | | |
| 1809/1810 | D731I | V63Q/L171P/H212A/S260T/E306G/ | + | + | | | | + | | |
| | | M320R/S332P/F387I/I390A/V393G/ | | | | | | | | |
| | | V534I/M558W/M681G/D731I/ | | | | | | | | |
| | | K732W/L749F/L750D/I775G/T1148N/ | | | | | | | | |
| | | L1248D/L1377D/V1439E | | | | | | | | |
| 1811/1812 | E918M | V63Q/L171P/H212A/S260T/E306G/ | ++ | + | | + | | + | | + |
| | | M320R/S332P/F387I/I390A/V393G/ | | | | | | | | |
| | | V534I/M558W/M681G/K732W/ | | | | | | | | |
| | | L749F/L750D/I775G/E918M/T1148N/ | | | | | | | | |
| | | L1248D/L1377D/V1439E | | | | | | | | |
| 1813/1814 | L1100S | V63Q/L171P/H212A/S260T/E306G/ | ++ | + | | + | + | + | + | + |
| | | M320R/S332P/F387I/I390A/V393G/ | | | | | | | | |
| | | V534I/M558W/M681G/K732W/ | | | | | | | | |
| | | L749F/L750D/I775G/L1100S/T1148N/ | | | | | | | | |
| | | L1248D/L1377D/V1439E | | | | | | | | |
| 1815/1816 | T1089S | V63Q/L171P/H212A/S260T/E306G/ | + | + | | + | + | + | + | |
| | | M320R/S332P/F387I/I390A/V393G/ | | | | | | | | |
| | | V534I/M558W/M681G/K732W/ | | | | | | | | |
| | | L749F/L750D/I775G/T1089S/T1148N/ | | | | | | | | |
| | | L1248D/L1377D/V1439E | | | | | | | | |
| 1817/1818 | H712Y | V63Q/L171P/H212A/S260T/E306G/ | + | + | + | + | + | + | + | + |
| | | M320R/S332P/F387I/I390A/V393G/ | | | | | | | | |
| | | V534I/M558W/M681G/H712Y/ | | | | | | | | |
| | | K732W/L749F/L750D/I775G/T1148N/ | | | | | | | | |
| | | L1248D/L1377D/V1439E | | | | | | | | |
| 1819/1820 | Y175H | V63Q/L171P/Y175H/H212A/S260T/ | + | + | + | | + | + | | |
| | | E306G/M320R/S332P/F387I/I390A/ | | | | | | | | |
| | | V393G/V534I/M558W/M681G/K732W/ | | | | | | | | |
| | | L749F/L750D/I775G/T1148N/ | | | | | | | | |
| | | L1248D/L1377D/V1439E | | | | | | | | |
| 1821/1822 | K1092G | V63Q/L171P/H212A/S260T/E306G/ | | + | ++ | | | + | | + |
| | | M320R/S332P/F387I/I390A/V393G/ | | | | | | | | |
| | | V534I/M558W/M681G/K732W/ | | | | | | | | |
| | | L749F/L750D/I775G/K1092G/T1148N/ | | | | | | | | |
| | | L1248D/L1377D/V1439E | | | | | | | | |
| 1823/1824 | T939E | V63Q/L171P/H212A/S260T/E306G/ | | | + | | | | | |
| | | M320R/S332P/F387I/I390A/V393G/ | | | | | | | | |
| | | V534I/M558W/M681G/K732W/ | | | | | | | | |
| | | L749F/L750D/I775G/T939E/T1148N/ | | | | | | | | |
| | | L1248D/L1377D/V1439E | | | | | | | | |
| 1825/1826 | Y1104E | V63Q/L171P/H212A/S260T/E306G/ | + | + | + | | | + | | |
| | | M320R/S332P/F387I/I390A/V393G/ | | | | | | | | |
| | | V534I/M558W/M681G/K732W/ | | | | | | | | |
| | | L749F/L750D/I775G/Y1104E/T1148N/ | | | | | | | | |
| | | L1248D/L1377D/V1439E | | | | | | | | |
| 1827/1828 | E800G | V63Q/L171P/H212A/S260T/E306G/ | ++ | + | ++ | ++ | + | + | + | + |
| | | M320R/S332P/F387I/I390A/V393G/ | | | | | | | | |
| | | V534I/M558W/M681G/K732W/ | | | | | | | | |
| | | L749F/L750D/I775G/E800G/T1148N/ | | | | | | | | |
| | | L1248D/L1377D/V1439E | | | | | | | | |
| 1829/1830 | D736F | V63Q/L171P/H212A/S260T/E306G/ | + | + | ++ | | + | | | |
| | | M320R/S332P/F387I/I390A/V393G/ | | | | | | | | |
| | | V534I/M558W/M681G/K732W/ | | | | | | | | |
| | | D736F/L749F/L750D/I775G/T1148N/ | | | | | | | | |
| | | L1248D/L1377D/V1439E | | | | | | | | |
| 1831/1832 | S543W | V63Q/L171P/H212A/S260T/E306G/ | | + | + | | ++ | + | + | |
| | | M320R/S332P/F387I/I390A/V393G/ | | | | | | | | |
| | | V534I/S543W/M558W/M681G/ | | | | | | | | |
| | | K732W/L749F/L750D/I775G/T1148N/ | | | | | | | | |
| | | L1248D/L1377D/V1439E | | | | | | | | |
| 1833/1834 | N1297A | V63Q/L171P/H212A/S260T/E306G/ | ++ | + | + | | ++ | ++ | + | ++ |
| | | M320R/S332P/F387I/I390A/V393G/ | | | | | | | | |
| | | V534I/M558W/M681G/K732W/ | | | | | | | | |
| | | L749F/L750D/I775G/T1148N/L1248D/ | | | | | | | | |
| | | N1297A/L1377D/V1439E | | | | | | | | |
| 1835/1836 | H712L | V63Q/L171P/H212A/S260T/E306G/ | + | + | + | | + | + | + | + |
| | | M320R/S332P/F387I/I390A/V393G/ | | | | | | | | |
| | | V534I/M558W/M681G/H712L/ | | | | | | | | |
| | | K732W/L749F/L750D/I775G/T1148N/ | | | | | | | | |
| | | L1248D/L1377D/V1439E | | | | | | | | |
| 1837/1838 | I1127V | V63Q/L171P/H212A/S260T/E306G/ | ++ | + | ++ | | | + | + | |
| | | M320R/S332P/F387I/I390A/V393G/ | | | | | | | | |
| | | V534I/M558W/M681G/K732W/ | | | | | | | | |
| | | L749F/L750D/I775G/I1127V/T1148N/ | | | | | | | | |
| | | L1248D/L1377D/V1439E | | | | | | | | |
| 1839/1840 | T939S | V63Q/L171P/H212A/S260T/E306G/ | + | + | + | + | + | + | + | + |
| | | M320R/S332P/F387I/I390A/V393G/ | | | | | | | | |
| | | V534I/M558W/M681G/K732W/ | | | | | | | | |
| | | L749F/L750D/I775G/T939S/T1148N/ | | | | | | | | |
| | | L1248D/L1377D/V1439E | | | | | | | | |
| 1841/1842 | L178M | V63Q/L171P/L178M/H212A/S260T/ | + | + | ++ | + | ++ | | ++ | |
| | | E306G/M320R/S332P/F387I/I390A/ | | | | | | | | |
| | | V393G/V534I/M558W/M681G/K732W/ | | | | | | | | |
| | | L749F/L750D/I775G/T1148N/ | | | | | | | | |
| | | L1248D/L1377D/V1439E | | | | | | | | |
| 1843/1844 | D731R | V63Q/L171P/H212A/S260T/E306G/ | ++ | + | + | | | + | | + |
| | | M320R/S332P/F387I/I390A/V393G/ | | | | | | | | |
| | | V534I/M558W/M681G/D731R/ | | | | | | | | |
| | | K732W/L749F/L750D/I775G/T1148N/ | | | | | | | | |
| | | L1248D/L1377D/V1439E | | | | | | | | |
| 1845/1846 | T686W | V63Q/L171P/H212A/S260T/E306G/ | + | + | | + | ++ | + | | |
| | | M320R/S332P/F387I/I390A/V393G/ | | | | | | | | |
| | | V534I/M558W/M681G/T686W/ | | | | | | | | |
| | | K732W/L749F/L750D/I775G/T1148N/ | | | | | | | | |
| | | L1248D/L1377D/V1439E | | | | | | | | |
| 1847/1848 | S543P | V63Q/L171P/H212A/S260T/E306G/ | ++ | + | + | ++ | +++ | ++ | ++ | ++ |
| | | M320R/S332P/F387I/I390A/V393G/ | | | | | | | | |
| | | V534I/S543P/M558W/M681G/ | | | | | | | | |
| | | K732W/L749F/L750D/I775G/T1148N/ | | | | | | | | |
| | | L1248D/L1377D/V1439E | | | | | | | | |
| 1849/1850 | D736G | V63Q/L171P/H212A/S260T/E306G/ | + | | + | | | | | |
| | | M320R/S332P/F387I/I390A/V393G/ | | | | | | | | |
| | | V534I/M558W/M681G/K732W/ | | | | | | | | |
| | | D736G/L749F/L750D/I775G/T1148N/ | | | | | | | | |
| | | L1248D/L1377D/V1439E | | | | | | | | |
| 1851/1852 | E1129P | V63Q/L171P/H212A/S260T/E306G/ | + | + | ++ | ++ | + | + | + | + |
| | | M320R/S332P/F387I/I390A/V393G/ | | | | | | | | |
| | | V534I/M558W/M681G/K732W/ | | | | | | | | |
| | | L749F/L750D/I775G/T1148N/E1129P/ | | | | | | | | |
| | | L1248D/L1377D/V1439E | | | | | | | | |
| 1853/1854 | L1138Q | V63Q/L171P/H212A/S260T/E306G/ | + | + | + | | + | + | | + |
| | | M320R/S332P/F387I/I390A/V393G/ | | | | | | | | |
| | | V534I/M558W/M681G/K732W/ | | | | | | | | |
| | | L749F/L750D/I775G/L1138Q/T1148N/ | | | | | | | | |
| | | L1248D/L1377D/V1439E | | | | | | | | |
| 1855/1856 | S812V | V63Q/L171P/H212A/S260T/E306G/ | ++ | + | | ++ | | + | + | ++ |
| | | M320R/S332P/F387I/I390A/V393G/ | | | | | | | | |
| | | V534I/M558W/M681G/K732W/ | | | | | | | | |
| | | L749F/L750D/I775G/S812V/T1148N/ | | | | | | | | |
| | | L1248D/L1377D/V1439E | | | | | | | | |
| 1857/1858 | E786D/ | V63Q/L171P/H212A/S260T/E306G/ | + | + | | + | | + | | + |
| | R821Q | M320R/S332P/F387I/I390A/V393G/ | | | | | | | | |
| | | V534I/M558W/M681G/K732W/ | | | | | | | | |
| | | L749F/L750D/I775G/E786D/R821Q/ | | | | | | | | |
| | | T1148N/L1248D/L1377D/V1439E | | | | | | | | |
| 1859/1860 | T535C | V63Q/L171P/H212A/S260T/E306G/ | | + | | | | + | | + |
| | | M320R/S332P/F387I/I390A/V393G/ | | | | | | | | |
| | | V534I/T535C/M558W/M681G/ | | | | | | | | |
| | | K732W/L749F/L750D/I775G/T1148N/ | | | | | | | | |
| | | L1248D/L1377D/V1439E | | | | | | | | |
| 1861/1862 | K680R | V63Q/L171P/H212A/S260T/E306G/ | + | + | + | ++ | | + | + | |
| | | M320R/S332P/F387I/I390A/V393G/ | | | | | | | | |
| | | V534I/M558W/K680R/M681G/ | | | | | | | | |
| | | K732W/L749F/L750D/I775G/T1148N/ | | | | | | | | |
| | | L1248D/L1377D/V1439E | | | | | | | | |
| 1863/1864 | S1116I | V63Q/L171P/H212A/S260T/E306G/ | + | + | + | + | ++ | ++ | ++ | |
| | | M320R/S332P/F387I/I390A/V393G/ | | | | | | | | |
| | | V534I/M558W/M681G/K732W/ | | | | | | | | |
| | | L749F/L750D/I775G/S1116I/T1148N/ | | | | | | | | |
| | | L1248D/L1377D/V1439E | | | | | | | | |
| 1865/1866 | T1089F | V63Q/L171P/H212A/S260T/E306G/ | + | + | + | + | | + | | + |
| | | M320R/S332P/F387I/I390A/V393G/ | | | | | | | | |
| | | V534I/M558W/M681G/K732W/ | | | | | | | | |
| | | L749F/L750D/I775G/T1089F/T1148N/ | | | | | | | | |
| | | L1248D/L1377D/V1439E | | | | | | | | |
| 1867/1868 | S304E | V63Q/L171P/H212A/S260T/S304E/ | ++ | + | ++ | + | | + | + | + |
| | | E306G/M320R/S332P/F3871/I390A/ | | | | | | | | |
| | | V393G/V534I/M558W/M681G/K732W/ | | | | | | | | |
| | | L749F/L750D/I775G/T1148N/ | | | | | | | | |
| | | L1248D/L1377D/V1439E | | | | | | | | |
| 1869/1870 | L178W | V63Q/L171P/L178W/H212A/S260T/ | + | + | | | | | | |
| | | E306G/M320R/S332P/F387I/I390A/ | | | | | | | | |
| | | V393G/V534I/M558W/M681G/K732W/ | | | | | | | | |
| | | L749F/L750D/I775G/T1148N/ | | | | | | | | |
| | | L1248D/L1377D/V1439E | | | | | | | | |
| 1871/1872 | Y1104C | V63Q/L171P/H212A/S260T/E306G/ | | | | | | + | | + |
| | | M320R/S332P/F387I/I390A/V393G/ | | | | | | | | |
| | | V534I/M558W/M681G/K732W/ | | | | | | | | |
| | | L749F/L750D/I775G/Y1104C/T1148N/ | | | | | | | | |
| | | L1248D/L1377D/V1439E | | | | | | | | |
| 1873/1874 | D744R | V63Q/L171P/H212A/S260T/E306G/ | + | | ++ | | + | | | |
| | | M320R/S332P/F387I/I390A/V393G/ | | | | | | | | |
| | | V534I/M558W/M681G/K732W/ | | | | | | | | |
| | | D744R/L749F/L750D/I775G/T1148N/ | | | | | | | | |
| | | L1248D/L1377D/V1439E | | | | | | | | |
| 1875/1876 | Y738R | V63Q/L171P/H212A/S260T/E306G/ | | | + | | ++ | | | |
| | | M320R/S332P/F387I/I390A/V393G/ | | | | | | | | |
| | | V534I/M558W/M681G/K732W/ | | | | | | | | |
| | | Y738R/L749F/L750D/I775G/T1148N/ | | | | | | | | |
| | | L1248D/L1377D/V1439E | | | | | | | | |
| 1877/1878 | K1093S | V63Q/L171P/H212A/S260T/E306G/ | ++ | ++ | ++ | ++ | + | ++ | ++ | + |
| | | M320R/S332P/F387I/I390A/V393G/ | | | | | | | | |
| | | V534I/M558W/M681G/K732W/ | | | | | | | | |
| | | L749F/L750D/I775G/K1093S/T1148N/ | | | | | | | | |
| | | L1248D/L1377D/V1439E | | | | | | | | |
| 1879/1880 | H712S | V63Q/L171P/H212A/S260T/E306G/ | ++ | + | ++ | ++ | | + | + | + |
| | | M320R/S332P/F387I/I390A/V393G/ | | | | | | | | |
| | | V534I/M558W/M681G/H712S/ | | | | | | | | |
| | | K732W/L749F/L750D/I775G/T1148N/ | | | | | | | | |
| | | L1248D/L1377D/V1439E | | | | | | | | |
| 1881/1882 | L187G | V63Q/L171P/L187G/H212A/S260T/ | | + | ++ | | | + | | |
| | | E306G/M320R/S332P/F387I/I390A/ | | | | | | | | |
| | | V393G/V534I/M558W/M681G/K732W/ | | | | | | | | |
| | | L749F/L750D/I775G/T1148N/ | | | | | | | | |
| | | L1248D/L1377D/V1439E | | | | | | | | |
| 1883/1884 | E132P | V63Q/L171P/H212A/S260T/E306G/ | + | | | + | ++ | | | |
| | | E132P/M320R/S332P/F387I/I390A/ | | | | | | | | |
| | | V393G/V534I/M558W/M681G/K732W/ | | | | | | | | |
| | | L749F/L750D/I775G/T1148N/ | | | | | | | | |
| | | L1248D/L1377D/V1439E | | | | | | | | |
| 1885/1886 | S812I | V63Q/L171P/H212A/S260T/E306G/ | + | + | ++ | | | | | |
| | | M320R/S332P/F387I/I390A/V393G/ | | | | | | | | |
| | | V534I/M558W/M681G/K732W/ | | | | | | | | |
| | | L749F/L750D/I775G/S812I/T1148N/ | | | | | | | | |
| | | L1248D/L1377D/V1439E | | | | | | | | |
| 1887/1888 | S1080G | V63Q/L171P/H212A/S260T/E306G/ | ++ | + | ++ | | + | + | + | |
| | | M320R/S332P/F387I/I390A/V393G/ | | | | | | | | |
| | | V534I/M558W/M681G/K732W/ | | | | | | | | |
| | | L749F/L750D/I775G/S1080G/T1148N/ | | | | | | | | |
| | | L1248D/L1377D/V1439E | | | | | | | | |
| 1889/1890 | E800C | V63Q/L171P/H212A/S260T/E306G/ | + | + | | ++ | | + | | + |
| | | M320R/S332P/F387I/I390A/V393G/ | | | | | | | | |
| | | V534I/M558W/M681G/K732W/ | | | | | | | | |
| | | L749F/L750D/I775G/E800C/T1148N/ | | | | | | | | |
| | | L1248D/L1377D/V1439E | | | | | | | | |
| 1891/1892 | Y1101G | V63Q/L171P/H212A/S260T/E306G/ | ++ | ++ | ++ | | + | ++ | + | |
| | | M320R/S332P/F387I/I390A/V393G/ | | | | | | | | |
| | | V534I/M558W/M681G/K732W/ | | | | | | | | |
| | | L749F/L750D/I775G/Y1101G/T1148N/ | | | | | | | | |
| | | L1248D/L1377D/V1439E | | | | | | | | |
| 1893/1894 | D808K | V63Q/L171P/H212A/S260T/E306G/ | + | ++ | + | ++ | + | + | | ++ |
| | | M320R/S332P/F387I/I390A/V393G/ | | | | | | | | |
| | | V534I/M558W/M681G/K732W/ | | | | | | | | |
| | | L749F/L750D/I775G/D808K/T1148N/ | | | | | | | | |
| | | L1248D/L1377D/V1439E | | | | | | | | |
| 1895/1896 | D808S | V63Q/L171P/H212A/S260T/E306G/ | ++ | + | ++ | + | + | + | + | + |
| | | M320R/S332P/F387I/I390A/V393G/ | | | | | | | | |
| | | V534I/M558W/M681G/K732W/ | | | | | | | | |
| | | L749F/L750D/I775G/D808S/T1148N/ | | | | | | | | |
| | | L1248D/L1377D/V1439E | | | | | | | | |
| 1897/1898 | D790A | V63Q/L171P/H212A/S260T/E306G/ | ++ | + | | | | + | | + |
| | | M320R/S332P/F387I/I390A/V393G/ | | | | | | | | |
| | | V534I/M558W/M681G/K732W/ | | | | | | | | |
| | | L749F/L750D/I775G/D790A/T1148N/ | | | | | | | | |
| | | L1248D/L1377D/V1439E | | | | | | | | |
| 1899/1900 | E800V | V63Q/L171P/H212A/S260T/E306G/ | ++ | + | ++ | +++ | ++ | ++ | ++ | + |
| | | M320R/S332P/F387I/I390A/V393G/ | | | | | | | | |
| | | V534I/M558W/M681G/K732W/ | | | | | | | | |
| | | L749F/L750D/I775G/E800V/T1148N/ | | | | | | | | |
| | | L1248D/L1377D/V1439E | | | | | | | | |
| 1901/1902 | K680L | V63Q/L171P/H212A/S260T/E306G/ | + | + | + | | | + | | ++ |
| | | M320R/S332P/F387I/I390A/V393G/ | | | | | | | | |
| | | V534I/M558W/K680L/M681G/ | | | | | | | | |
| | | K732W/L749F/L750D/I775G/T1148N/ | | | | | | | | |
| | | L1248D/L1377D/V1439E | | | | | | | | |
| 1903/1904 | A167R | V63Q/A167R/L171P/H212A/S260T/ | ++ | + | + | ++ | + | + | + | + |
| | | E306G/M320R/S332P/F387I/I390A/ | | | | | | | | |
| | | V393G/V534I/M558W/M681G/K732W/ | | | | | | | | |
| | | L749F/L750D/I775G/T1148N/ | | | | | | | | |
| | | L1248D/L1377D/V1439E | | | | | | | | |
| 1905/1906 | K752E | V63Q/L171P/H212A/S260T/E306G/ | ++ | ++ | ++ | ++ | + | ++ | ++ | |
| | | M320R/S332P/F387I/I390A/V393G/ | | | | | | | | |
| | | V534I/M558W/M681G/K732W/ | | | | | | | | |
| | | L749F/L750D/K752E/I775G/T1148N/ | | | | | | | | |
| | | L1248D/L1377D/V1439E | | | | | | | | |
| 1907/1908 | K1093N | V63Q/L171P/H212A/S260T/E306G/ | + | ++ | + | ++ | | ++ | + | + |
| | | M320R/S332P/F387I/I390A/V393G/ | | | | | | | | |
| | | V534I/M558W/M681G/K732W/ | | | | | | | | |
| | | L749F/L750D/I775G/K1093N/T1148N/ | | | | | | | | |
| | | L1248D/L1377D/V1439E | | | | | | | | |
| 1909/1910 | A119G | V63Q/A119G/L171P/H212A/S260T/ | ++ | + | ++ | | ++ | ++ | + | |
| | | E306G/M320R/S332P/F387I/I390A/ | | | | | | | | |
| | | V393G/V534I/M558W/M681G/K732W/ | | | | | | | | |
| | | L749F/L750D/I775G/T1148N/ | | | | | | | | |
| | | L1248D/L1377D/V1439E | | | | | | | | |
| 1911/1912 | E739G | V63Q/L171P/H212A/S260T/E306G/ | ++ | + | ++ | ++ | ++++ | + | ++ | ++ |
| | | M320R/S332P/F387I/I390A/V393G/ | | | | | | | | |
| | | V534I/M558W/M681G/K732W/ | | | | | | | | |
| | | E739G/L749F/L750D/I775G/T1148N/ | | | | | | | | |
| | | L1248D/L1377D/V1439E | | | | | | | | |
| 1913/1914 | S1116R | V63Q/L171P/H212A/S260T/E306G/ | + | + | ++ | + | + | + | + | + |
| | | M320R/S332P/F387I/I390A/V393G/ | | | | | | | | |
| | | V534I/M558W/M681G/K732W/ | | | | | | | | |
| | | L749F/L750D/I775G/S1116R/T1148N/ | | | | | | | | |
| | | L1248D/L1377D/V1439E | | | | | | | | |
| 1915/1916 | Q580Y | V63Q/L171P/H212A/S260T/E306G/ | + | + | ++ | ++ | | + | | + |
| | | M320R/S332P/F387I/I390A/V393G/ | | | | | | | | |
| | | V534I/M558W/Q580Y/M681G/ | | | | | | | | |
| | | K732W/L749F/L750D/I775G/T1148N/ | | | | | | | | |
| | | L1248D/L1377D/V1439E | | | | | | | | |
| 1917/1918 | S304A | V63Q/L171P/H212A/S260T/S304A/ | + | + | + | | ++ | ++ | + | |
| | | E306G/M320R/S332P/F387I/I390A/ | | | | | | | | |
| | | V393G/V534I/M558W/M681G/ | | | | | | | | |
| | | K732W/L749F/L750D/I775G/T1148N/ | | | | | | | | |
| | | L1248D/L1377D/V1439E | | | | | | | | |
| 1919/1920 | H712Q | V63Q/L171P/H212A/S260T/E306G/ | ++ | + | + | | ++ | ++ | ++ | |
| | | M320R/S332P/F387I/I390A/V393G/ | | | | | | | | |
| | | V534I/M558W/M681G/H712Q/ | | | | | | | | |
| | | K732W/L749F/L750D/I775G/T1148N/ | | | | | | | | |
| | | L1248D/L1377D/V1439E | | | | | | | | |
| 1921/1922 | D806S | V63Q/L171P/H212A/S260T/E306G/ | + | + | ++ | ++ | + | + | | |
| | | M320R/S332P/F387I/I390A/V393G/ | | | | | | | | |
| | | V534I/M558W/M681G/K732W/ | | | | | | | | |
| | | L749F/L750D/I775G/D806S/T1148N/ | | | | | | | | |
| | | L1248D/L1377D/V1439E | | | | | | | | |
| 1923/1924 | H712G | V63Q/L171P/H212A/S260T/E306G/ | + | + | + | + | + | ++ | + | |
| | | M320R/S332P/F387I/I390A/V393G/ | | | | | | | | |
| | | V534I/M558W/M681G/H712G/ | | | | | | | | |
| | | K732W/L749F/L750D/I775G/T1148N/ | | | | | | | | |
| | | L1248D/L1377D/V1439E | | | | | | | | |
| 1925/1926 | D790G | V63Q/L171P/H212A/S260T/E306G/ | + | + | + | + | | + | | |
| | | M320R/S332P/F387I/I390A/V393G/ | | | | | | | | |
| | | V534I/M558W/M681G/K732W/ | | | | | | | | |
| | | L749F/L750D/I775G/D790G/T1148N/ | | | | | | | | |
| | | L1248D/L1377D/V1439E | | | | | | | | |
| 1927/1928 | F1108V | V63Q/L171P/H212A/S260T/E306G/ | + | + | + | ++ | | + | + | ++ |
| | | M320R/S332P/F387I/I390A/V393G/ | | | | | | | | |
| | | V534I/M558W/M681G/K732W/ | | | | | | | | |
| | | L749F/L750D/I775G/F1108V/T1148N/ | | | | | | | | |
| | | L1248D/L1377D/V1439E | | | | | | | | |
| 1929/1930 | D731L | V63Q/L171P/H212A/S260T/E306G/ | + | + | + | ++ | ++ | + | + | + |
| | | M320R/S332P/F387I/I390A/V393G/ | | | | | | | | |
| | | V534I/M558W/M681G/D731L/ | | | | | | | | |
| | | K732W/L749F/L750D/I775G/T1148N/ | | | | | | | | |
| | | L1248D/L1377D/V1439E | | | | | | | | |
| 1931/1932 | S1084N | V63Q/L171P/H212A/S260T/E306G/ | ++ | + | | ++ | + | + | | + |
| | | M320R/S332P/F387I/I390A/V393G/ | | | | | | | | |
| | | V534I/M558W/M681G/K732W/ | | | | | | | | |
| | | L749F/L750D/I775G/S1084N/T1148N/ | | | | | | | | |
| | | L1248D/L1377D/V1439E | | | | | | | | |
| 1933/1934 | I1127L | V63Q/L171P/H212A/S260T/E306G/ | + | + | ++ | ++ | | + | | + |
| | | M320R/S332P/F387I/I390A/V393G/ | | | | | | | | |
| | | V534I/M558W/M681G/K732W/ | | | | | | | | |
| | | L749F/L750D/I775G/I1127L/T1148N/ | | | | | | | | |
| | | L1248D/L1377D/V1439E | | | | | | | | |
| 1935/1936 | S308P | V63Q/L171P/H212A/S260T/E306G/ | + | + | + | + | | + | + | + |
| | | S308P/M320R/S332P/F387I/I390A/ | | | | | | | | |
| | | V393G/V534I/M558W/M681G/K732W/ | | | | | | | | |
| | | L749F/L750D/I775G/T1148N/ | | | | | | | | |
| | | L1248D/L1377D/V1439E | | | | | | | | |
| 1937/1938 | E1076S | V63Q/L171P/H212A/S260T/E306G/ | ++ | + | ++ | ++ | ++ | ++ | ++ | ++ |
| | | M320R/S332P/F387I/I390A/V393G/ | | | | | | | | |
| | | V534I/M558W/M681G/K732W/ | | | | | | | | |
| | | L749F/L750D/I775G/E1076S/T1148N/ | | | | | | | | |
| | | L1248D/L1377D/V1439E | | | | | | | | |
| 1939/1940 | K1093P | V63Q/L171P/H212A/S260T/E306G/ | + | ++ | ++ | + | | + | | + |
| | | M320R/S332P/F387I/I390A/V393G/ | | | | | | | | |
| | | V534I/M558W/M681G/K732W/ | | | | | | | | |
| | | L749F/L750D/I775G/K1093P/T1148N/ | | | | | | | | |
| | | L1248D/L1377D/V1439E | | | | | | | | |
| 1941/1942 | K1093Y | V63Q/L171P/H212A/S260T/E306G/ | + | ++ | + | ++ | + | ++ | + | ++ |
| | | M320R/S332P/F387I/I390A/V393G/ | | | | | | | | |
| | | V534I/M558W/M681G/K732W/ | | | | | | | | |
| | | L749F/L750D/I775G/K1093Y/T1148N/ | | | | | | | | |
| | | L1248D/L1377D/V1439E | | | | | | | | |
| 1943/1944 | E739R | V63Q/L171P/H212A/S260T/E306G/ | | | + | | + | | | |
| | | M320R/S332P/F387I/I390A/V393G/ | | | | | | | | |
| | | V534I/M558W/M681G/K732W/ | | | | | | | | |
| | | E739R/L749F/L750D/I775G/T1148N/ | | | | | | | | |
| | | L1248D/L1377D/V1439E | | | | | | | | |
| 1945/1946 | E739P | V63Q/L171P/H212A/S260T/E306G/ | + | + | + | + | + | + | + | + |
| | | M320R/S332P/F387I/I390A/V393G/ | | | | | | | | |
| | | V534I/M558W/M681G/K732W/ | | | | | | | | |
| | | E739P/L749F/L750D/I775G/T1148N/ | | | | | | | | |
| | | L1248D/L1377D/V1439E | | | | | | | | |
| 1947/1948 | I310V | V63Q/L171P/H212A/S260T/E306G/ | ++ | + | ++ | ++ | + | + | | |
| | | I310V/M320R/S332P/F387I/I390A/ | | | | | | | | |
| | | V393G/V534I/M558W/M681G/K732W/ | | | | | | | | |
| | | L749F/L750D/I775G/T1148N/ | | | | | | | | |
| | | L1248D/L1377D/V1439E | | | | | | | | |
| 1949/1950 | H712T | V63Q/L171P/H212A/S260T/E306G/ | | + | | | | | | |
| | | M320R/S332P/F387I/I390A/V393G/ | | | | | | | | |
| | | V534I/M558W/M681G/H712T/ | | | | | | | | |
| | | K732W/L749F/L750D/I775G/T1148N/ | | | | | | | | |
| | | L1248D/L1377D/V1439E | | | | | | | | |
| 1951/1952 | A167H | V63Q/A167H/L171P/H212A/S260T/ | + | + | + | + | + | + | + | |
| | | E306G/M320R/S332P/F387I/I390A/ | | | | | | | | |
| | | V393G/V534I/M558W/M681G/K732W/ | | | | | | | | |
| | | L749F/L750D/I775G/T1148N/ | | | | | | | | |
| | | L1248D/L1377D/V1439E | | | | | | | | |
| 1953/1954 | K1093R | V63Q/L171P/H212A/S260T/E306G/ | + | + | + | ++ | + | + | + | |
| | | M320R/S332P/F387I/I390A/V393G/ | | | | | | | | |
| | | V534I/M558W/M681G/K732W/ | | | | | | | | |
| | | L749F/L750D/I775G/K1093R/T1148N/ | | | | | | | | |
| | | L1248D/L1377D/V1439E | | | | | | | | |
| 1955/1956 | V536P/F555E/ | V63Q/L171P/H212A/S260T/E306G/ | ++ | + | ++++ | | + | + | ++ | |
| | G619E/ | M320R/S332P/F387I/I390A/V393G/ | | | | | | | | |
| | W732K/K799S/ | V534I/V536P/F555E/M558W/G619E/ | | | | | | | | |
| | Y805Q | M681G/L749F/L750D/I775G/ | | | | | | | | |
| | | K799S/Y805Q/T1148N/L1248D/ | | | | | | | | |
| | | L1377D/V1439E | | | | | | | | |
| 1957/1958 | F555E/G619E/ | V63Q/L171P/H212A/S260T/E306G/ | ++ | ++ | ++ | ++ | + | ++ | + | |
| | D801G | M320R/S332P/F387I/I390A/V393G/ | | | | | | | | |
| | | V534I/F555E/M558W/G619E/M681G/ | | | | | | | | |
| | | K732W/L749F/L750D/I775G/ | | | | | | | | |
| | | D801G/T1148N/L1248D/L1377D/ | | | | | | | | |
| | | V1439E | | | | | | | | |
| 1959/1960 | F555E/G619E/ | V63Q/L171P/H212A/S260T/E306G/ | ++ | ++ | ++ | ++ | ++ | ++ | ++ | + |
| | K799S | M320R/S332P/F387I/I390A/V393G/ | | | | | | | | |
| | | V534I/F555E/M558W/G619E/M681G/ | | | | | | | | |
| | | K732W/L749F/L750D/I775G/ | | | | | | | | |
| | | K799S/T1148N/L1248D/L1377D/ | | | | | | | | |
| | | V1439E | | | | | | | | |
| 1961/1962 | G619E/K799S/ | V63Q/L171P/H212A/S260T/E306G/ | ++ | ++ | ++ | ++ | ++ | + | ++ | |
| | D801G/ | M320R/S332P/F387I/I390A/V393G/ | | | | | | | | |
| | Y805Q | V534I/M558W/G619E/M681G/ | | | | | | | | |
| | | K732W/L749F/L750D/I775G/K799S/ | | | | | | | | |
| | | D801G/Y805Q/T1148N/L1248D/ | | | | | | | | |
| | | L1377D/V1439E | | | | | | | | |
| 1963/1964 | V536P/F555E/ | V63Q/L171P/H212A/S260T/E306G/ | ++ | + | ++ | | ++ | + | + | + |
| | G619E/ | M320R/S332P/F387I/I390A/V393G/ | | | | | | | | |
| | W732K/K799S | V534I/V536P/F555E/M558W/G619E/ | | | | | | | | |
| | | M681G/L749F/L750D/I775G/ | | | | | | | | |
| | | K799S/T1148N/L1248D/L1377D/ | | | | | | | | |
| | | V1439E | | | | | | | | |
| 1965/1966 | K799S/ | V63Q/L171P/H212A/S260T/E306G/ | ++ | + | ++ | + | ++ | + | + | |
| | Y805Q | M320R/S332P/F387I/I390A/V393G/ | | | | | | | | |
| | | V534I/M558W/M681G/K732W/ | | | | | | | | |
| | | L749F/L750D/I775G/K799S/Y805Q/ | | | | | | | | |
| | | T1148N/L1248D/L1377D/V1439E | | | | | | | | |
| | | V63Q/ | | | | | | | | |
| 1967/1968 | D801G | L171P/H212A/S260T/E306G/ | ++ | + | ++ | + | + | + | | |
| | | M320R/S332P/F387I/I390A/V393G/ | | | | | | | | |
| | | V534I/M558W/M681G/K732W/ | | | | | | | | |
| | | L749F/L750D/I775G/D801G/T1148N/ | | | | | | | | |
| | | L1248D/L1377D/V1439E | | | | | | | | |
| 1969/1970 | A246D/G619E/ | V63Q/L171P/H212A/A246D/S260T/ | ++ | ++ | ++ | ++ | | + | | + |
| | K799S/D801G | E306G/M320R/S332P/F387I/I390A/ | | | | | | | | |
| | | V393G/V534I/M558W/G619E/M681G/ | | | | | | | | |
| | | K732W/L749F/L750D/I775G/ | | | | | | | | |
| | | K799S/D801G/T1148N/L1248D/ | | | | | | | | |
| | | L1377D/V1439E | | | | | | | | |
| 1971/1972 | V536P/F555E/ | V63Q/L171P/H212A/S260T/E306G/ | ++ | ++ | ++ | ++ | ++ | + | + | + |
| | G619E | M320R/S332P/F387I/I390A/V393G/ | | | | | | | | |
| | | V534I/V536P/F555E/M558W/G619E/ | | | | | | | | |
| | | M681G/K732W/L749F/L750D/ | | | | | | | | |
| | | I775G/T1148N/L1248D/L1377D/ | | | | | | | | |
| | | V1439E | | | | | | | | |
| 1973/1974 | G619E/S839K | V63Q/L171P/H212A/S260T/E306G/ | ++ | + | ++ | | + | | ++ | + |
| | | M320R/S332P/F387I/I390A/V393G/ | | | | | | | | |
| | | V534I/M558W/G619E/M681G/ | | | | | | | | |
| | | K732W/L749F/L750D/I775G/S839K/ | | | | | | | | |
| | | T1148N/L1248D/L1377D/V1439E | | | | | | | | |
| 1975/1976 | G619E/K799S/ | V63Q/L171P/H212A/S260T/E306G/ | ++ | ++ | ++ | ++ | + | + | | |
| | Y805Q | M320R/S332P/F387I/I390A/V393G/ | | | | | | | | |
| | | V534I/M558W/G619E/M681G/ | | | | | | | | |
| | | K732W/L749F/L750D/I775G/K799S/ | | | | | | | | |
| | | Y805Q/T1148N/L1248D/L1377D/ | | | | | | | | |
| | | V1439E | | | | | | | | |
| 1977/1978 | V536P/D801G/ | V63Q/L171P/H212A/S260T/E306G/ | ++ | + | ++ | ++ | + | + | + | + |
| | Y805Q | M320R/S332P/F387I/I390A/V393G/ | | | | | | | | |
| | | V534I/V536P/M558W/M681G/ | | | | | | | | |
| | | K732W/L749F/L750D/I775G/D801G/ | | | | | | | | |
| | | Y805Q/T1148N/L1248D/L1377D/ | | | | | | | | |
| | | V1439E | | | | | | | | |
| 1979/1980 | F555E/G619E/ | V63Q/L171P/H212A/S260T/E306G/ | ++ | ++ | ++ | ++ | + | + | + | + |
| | K799S/ | M320R/S332P/F387I/I390A/V393G/ | | | | | | | | |
| | Y805Q | V534I/F555E/M558W/G619E/M681G/ | | | | | | | | |
| | | K732W/L749F/L750D/I775G/ | | | | | | | | |
| | | K799S/Y805Q/T1148N/L1248D/ | | | | | | | | |
| | | L1377D/V1439E | | | | | | | | |
| 1981/1982 | G619E/D801G/ | V63Q/L171P/H212A/S260T/E306G/ | ++ | ++ | ++ | ++ | + | + | | + |
| | Y805Q | M320R/S332P/F387I/I390A/V393G/ | | | | | | | | |
| | | V534I/M558W/G619E/M681G/ | | | | | | | | |
| | | K732W/L749F/L750D/I775G/D801G/ | | | | | | | | |
| | | Y805Q/T1148N/L1248D/L1377D/ | | | | | | | | |
| | | V1439E | | | | | | | | |
| 1983/1984 | F555E/ | V63Q/L171P/H212A/S260T/E306G/ | ++ | + | + | ++ | + | + | | + |
| | G619E | M320R/S332P/F387I/I390A/V393G/ | | | | | | | | |
| | | V534I/F555E/M558W/G619E/M681G/ | | | | | | | | |
| | | K732W/L749F/L750D/I775G/ | | | | | | | | |
| | | T1148N/L1248D/L1377D/V1439E | | | | | | | | |
| 1985/1986 | V536P/G619E/ | V63Q/L171P/H212A/S260T/E306G/ | ++ | ++ | ++ | ++ | + | + | + | + |
| | D801G | M320R/S332P/F387I/I390A/V393G/ | | | | | | | | |
| | | V534I/V536P/M558W/G619E/M681G/ | | | | | | | | |
| | | K732W/L749F/L750D/I775G/ | | | | | | | | |
| | | D801G/T1148N/L1248D/L1377D/ | | | | | | | | |
| | | V1439E | | | | | | | | |
| 1987/1988 | D801G/ | V63Q/L171P/H212A/S260T/E306G/ | + | + | ++ | + | ++ | + | + | + |
| | Y805Q | M320R/S332P/F387I/I390A/V393G/ | | | | | | | | |
| | | V534I/M558W/M681G/K732W/ | | | | | | | | |
| | | L749F/L750D/I775G/D801G/Y805Q/ | | | | | | | | |
| | | T1148N/L1248D/L1377D/V1439E | | | | | | | | |
| 1989/1990 | V536P/G619E/ | V63Q/L171P/H212A/S260T/E306G/ | ++ | ++ | ++ | ++ | + | + | + | |
| | K799S/ | M320R/S332P/F387I/I390A/V393G/ | | | | | | | | |
| | Y805Q | V534I/V536P/M558W/G619E/M681G/ | | | | | | | | |
| | | K732W/L749F/L750D/I775G/ | | | | | | | | |
| | | K799S/Y805Q/T1148N/L1248D/ | | | | | | | | |
| | | L1377D/V1439E | | | | | | | | |
| 1991/1992 | F555E/G619E/ | V63Q/L171P/H212A/S260T/E306G/ | ++ | + | ++ | + | ++ | + | + | |
| | W732K/K799S/ | M320R/S332P/F387I/I390A/V393G/ | | | | | | | | |
| | D801G/ | V534I/F555E/M558W/G619E/M681G/ | | | | | | | | |
| | Y805Q | L749F/L750D/I775G/K799S/ | | | | | | | | |
| | | D801G/Y805Q/T1148N/L1248D/ | | | | | | | | |
| | | L1377D/V1439E | | | | | | | | |
| 1993/1994 | Y1281H | V63Q/L171P/H212A/S260T/E306G/ | | + | | | + | + | | |
| | | M320R/S332P/F387I/I390A/V393G/ | | | | | | | | |
| | | V534I/M558W/M681G/K732W/ | | | | | | | | |
| | | L749F/L750D/I775G/T1148N/L1248D/ | | | | | | | | |
| | | Y1281H/L1377D/V1439E | | | | | | | | |
| 1995/1996 | F749L | V63Q/L171P/H212A/S260T/E306G/ | + | | ++ | | + | | ++ | |
| | | M320R/S332P/F387I/I390A/V393G/ | | | | | | | | |
| | | V534I/M558W/M681G/K732W/ | | | | | | | | |
| | | L750D/I775G/T1148N/L1248D/L1377D/ | | | | | | | | |
| | | V1439E | | | | | | | | |
| 1997/1998 | L614H | V63Q/L171P/H212A/S260T/E306G/ | | + | | + | | | | |
| | | M320R/S332P/F387I/I390A/V393G/ | | | | | | | | |
| | | V534I/M558W/L614H/M681G/ | | | | | | | | |
| | | K732W/L749F/L750D/I775G/T1148N/ | | | | | | | | |
| | | L1248D/L1377D/V1439E | | | | | | | | |
| 1999/2000 | N616D | V63Q/L171P/H212A/S260T/E306G/ | + | + | + | + | + | + | + | + |
| | | M320R/S332P/F387I/I390A/V393G/ | | | | | | | | |
| | | V534I/M558W/N616D/M681G/ | | | | | | | | |
| | | K732W/L749F/L750D/I775G/T1148N/ | | | | | | | | |
| | | L1248D/L1377D/V1439E | | | | | | | | |
| 2001/2002 | Q621K | V63Q/L171P/H212A/S260T/E306G/ | + | + | | + | ++ | ++ | + | |
| | | M320R/S332P/F387I/I390A/V393G/ | | | | | | | | |
| | | V534I/M558W/Q621K/M681G/ | | | | | | | | |
| | | K732W/L749F/L750D/I775G/T1148N/ | | | | | | | | |
| | | L1248D/L1377D/V1439E | | | | | | | | |
| 2003/2004 | I1050E | V63Q/L171P/H212A/S260T/E306G/ | ++ | + | + | | + | + | | + |
| | | M320R/S332P/F387I/I390A/V393G/ | | | | | | | | |
| | | V534I/M558W/M681G/K732W/ | | | | | | | | |
| | | L749F/L750D/I775G/I1050E/T1148N/ | | | | | | | | |
| | | L1248D/L1377D/V1439E | | | | | | | | |
| 2005/2006 | I610H | V63Q/L171P/H212A/S260T/E306G/ | + | + | | | | + | | |
| | | M320R/S332P/F387I/I390A/V393G/ | | | | | | | | |
| | | V534I/M558W/I610H/M681G/ | | | | | | | | |
| | | K732W/L749F/L750D/I775G/T1148N/ | | | | | | | | |
| | | L1248D/L1377D/V1439E | | | | | | | | |
| 2007/2008 | Q611D | V63Q/L171P/H212A/S260T/E306G/ | + | + | + | + | | + | | |
| | | M320R/S332P/F387I/I390A/V393G/ | | | | | | | | |
| | | V534I/M558W/Q611D/M681G/ | | | | | | | | |
| | | K732W/L749F/L750D/I775G/T1148N/ | | | | | | | | |
| | | L1248D/L1377D/V1439E | | | | | | | | |
| 2009/2010 | R1042T | V63Q/L171P/H212A/S260T/E306G/ | + | | | | + | | + | |
| | | M320R/S332P/F387I/I390A/V393G/ | | | | | | | | |
| | | V534I/M558W/M681G/K732W/ | | | | | | | | |
| | | L749F/L750D/I775G/R1042T/T1148N/ | | | | | | | | |
| | | L1248D/L1377D/V1439E | | | | | | | | |
| 2011/2012 | N616Q | V63Q/L171P/H212A/S260T/E306G/ | ++ | + | + | | ++ | + | | |
| | | M320R/S332P/F387I/I390A/V393G/ | | | | | | | | |
| | | V534I/M558W/N616Q/M681G/ | | | | | | | | |
| | | K732W/L749F/L750D/I775G/T1148N/ | | | | | | | | |
| | | L1248D/L1377D/V1439E | | | | | | | | |
| 2013/2014 | L614N | V63Q/L171P/H212A/S260T/E306G/ | + | + | | ++ | + | + | | + |
| | | M320R/S332P/F387I/I390A/V393G/ | | | | | | | | |
| | | V534I/M558W/L614N/M681G/ | | | | | | | | |
| | | K732W/L749F/L750D/I775G/T1148N/ | | | | | | | | |
| | | L1248D/L1377D/V1439E | | | | | | | | |
| 2015/2016 | T370A/ | V63Q/L171P/H212A/S260T/E306G/ | + | + | ++ | ++ | + | + | | |
| | L614A | M320R/S332P/T370A/F387I/I390A/ | | | | | | | | |
| | | V393G/V534I/M558W/L614A/ | | | | | | | | |
| | | M681G/K732W/L749F/L750D/I775G/ | | | | | | | | |
| | | T1148N/L1248D/L1377D/V1439E | | | | | | | | |
| 2017/2018 | H1156D | V63Q/L171P/H212A/S260T/E306G/ | + | + | + | | | + | + | + |
| | | M320R/S332P/F387I/I390A/V393G/ | | | | | | | | |
| | | V534I/M558W/M681G/K732W/ | | | | | | | | |
| | | L749F/L750D/I775G/T1148N/H1156D/ | | | | | | | | |
| | | L1248D/L1377D/V1439E | | | | | | | | |
| 2019/2020 | K1210V | V63Q/L171P/H212A/S260T/E306G/ | + | + | + | + | ++ | + | + | |
| | | M320R/S332P/F387I/I390A/V393G/ | | | | | | | | |
| | | V534I/M558W/M681G/K732W/ | | | | | | | | |
| | | L749F/L750D/I775G/T1148N/K1210V/ | | | | | | | | |
| | | L1248D/L1377D/V1439E | | | | | | | | |
| 2021/2022 | L614T | V63Q/L171P/H212A/S260T/E306G/ | ++ | + | + | | | + | + | + |
| | | M320R/S332P/F387I/I390A/V393G/ | | | | | | | | |
| | | V534I/M558W/L614T/M681G/ | | | | | | | | |
| | | K732W/L749F/L750D/I775G/T1148N/ | | | | | | | | |
| | | L1248D/L1377D/V1439E | | | | | | | | |
| 2023/2024 | R1158N | V63Q/L171P/H212A/S260T/E306G/ | + | + | | + | | | | |
| | | M320R/S332P/F387I/I390A/V393G/ | | | | | | | | |
| | | V534I/M558W/M681G/K732W/ | | | | | | | | |
| | | L749F/L750D/I775G/T1148N/R1158N/ | | | | | | | | |
| | | L1248D/L1377D/V1439E | | | | | | | | |
| 2025/2026 | Y1281S | V63Q/L171P/H212A/S260T/E306G/ | + | + | + | + | + | + | | + |
| | | M320R/S332P/F387I/I390A/V393G/ | | | | | | | | |
| | | V534I/M558W/M681G/K732W/ | | | | | | | | |
| | | L749F/L750D/I775G/T1148N/L1248D/ | | | | | | | | |
| | | Y1281S/L1377D/V1439E | | | | | | | | |
| 2027/2028 | A618E | V63Q/L171P/H212A/S260T/E306G/ | ++ | + | + | + | + | + | + | + |
| | | M320R/S332P/F387I/I390A/V393G/ | | | | | | | | |
| | | V534I/M558W/A618E/M681G/ | | | | | | | | |
| | | K732W/L749F/L750D/I775G/T1148N/ | | | | | | | | |
| | | L1248D/L1377D/V1439E | | | | | | | | |
| 2029/2030 | F555E/G681N/ | V63Q/L171P/H212A/S260T/E306G/ | + | | | + | ++ | + | + | |
| | Y737K/ | M320R/S332P/F387I/I390A/V393G/ | | | | | | | | |
| | K799Q | V534I/F555E/M558W/M681N/ | | | | | | | | |
| | | K732W/Y737K/L749F/L750D/I775G/ | | | | | | | | |
| | | K799Q/T1148N/L1248D/L1377D/ | | | | | | | | |
| | | V1439E | | | | | | | | |
| 2031/2032 | V536P/G619E/ | V63Q/L171P/H212A/S260T/E306G/ | ++ | ++ | ++ | ++ | ++ | ++ | + | |
| | E809G/ | M320R/S332P/F387I/I390A/V393G/ | | | | | | | | |
| | E1109G | V534I/V536P/M558W/G619E/M681G/ | | | | | | | | |
| | | K732W/L749F/L750D/I775G/ | | | | | | | | |
| | | E809G/E1109G/T1148N/L1248D/ | | | | | | | | |
| | | L1377D/V1439E | | | | | | | | |
| 2033/2034 | V536K/F555G/ | V63Q/L171P/H212A/S260T/E306G/ | ++ | | ++ | | | | | |
| | G619E/Y737K/ | M320R/S332P/F387I/I390A/V393G/ | | | | | | | | |
| | K799Q/ | V534I/V536K/F555G/M558W/ | | | | | | | | |
| | E809G/S839K/ | G619E/M681G/K732W/Y737K/L749F/ | | | | | | | | |
| | S916T/Q921R | L750D/I775G/K799Q/E809G/ | | | | | | | | |
| | | S839K/S916T/Q921R/T1148N/L1248D/ | | | | | | | | |
| | | L1377D/V1439E | | | | | | | | |
| 2035/2036 | S916T/ | V63Q/L171P/H212A/S260T/E306G/ | + | + | ++ | ++ | + | + | + | |
| | E1109G | M320R/S332P/F387I/I390A/V393G/ | | | | | | | | |
| | | V534I/M558W/M681G/K732W/ | | | | | | | | |
| | | L749F/L750D/I775G/S916T/E1109G/ | | | | | | | | |
| | | T1148N/L1248D/L1377D/V1439E | | | | | | | | |
| 2037/2038 | V536R/G619E/ | V63Q/L171P/H212A/S260T/E306G/ | ++ | + | ++ | ++ | | + | | |
| | G681N/K799Q/ | M320R/S332P/F387I/I390A/V393G/ | | | | | | | | |
| | S916T/ | V534I/V536R/M558W/G619E/ | | | | | | | | |
| | E1109G | M681N/K732W/L749F/L750D/I775G/ | | | | | | | | |
| | | K799Q/S916T/E1109G/T1148N/ | | | | | | | | |
| | | L1248D/L1377D/V1439E | | | | | | | | |
| 2039/2040 | F555G/G619E/ | V63Q/L171P/H212A/S260T/E306G/ | ++ | ++ | ++ | ++ | + | ++ | + | ++ |
| | G681N/E807R/ | M320R/S332P/F387I/I390A/V393G/ | | | | | | | | |
| | E1109G | V534I/F555G/M558W/G619E/M681N/ | | | | | | | | |
| | | K732W/L749F/L750D/I775G/ | | | | | | | | |
| | | E807R/E1109G/T1148N/L1248D/ | | | | | | | | |
| | | L1377D/V1439E | | | | | | | | |
| 2041/2042 | F555E/G619E/ | V63Q/L171P/H212A/S260T/E306G/ | ++ | | ++ | | + | | + | |
| | G681N/Y737K/ | M320R/S332P/F387I/I390A/V393G/ | | | | | | | | |
| | K799Q | V534I/F555E/M558W/G619E/M681N/ | | | | | | | | |
| | | L749F/L750D/I775G/K799Q/ | | | | | | | | |
| | | T1148N/L1248D/L1377D/V1439E | | | | | | | | |
| 2043/2044 | G619E/G681N/ | V63Q/L171P/H212A/S260T/E306G/ | ++ | ++ | ++ | ++ | + | ++ | + | ++ |
| | K799S/S916T/ | M320R/S332P/F387I/I390A/V393G/ | | | | | | | | |
| | E1109G | V534I/M558W/G619E/M681N/ | | | | | | | | |
| | | K732W/L749F/L750D/I775G/K799S/ | | | | | | | | |
| | | S916T/E1109G/T1148N/L1248D/ | | | | | | | | |
| | | L1377D/V1439E | | | | | | | | |
| 2045/2046 | G619E/G681N | V63Q/L171P/H212A/S260T/E306G/ | ++ | ++ | ++ | +++ | + | ++ | + | ++ |
| | | M320R/S332P/F387I/I390A/V393G/ | | | | | | | | |
| | | V534I/M558W/G619E/M681N/ | | | | | | | | |
| | | K732W/L749F/L750D/I775G/T1148N/ | | | | | | | | |
| | | L1248D/L1377D/V1439E | | | | | | | | |
| 2047/2048 | F555G/Q921R | V63Q/L171P/H212A/S260T/E306G/ | + | + | ++ | + | | + | + | |
| | | M320R/S332P/F387I/I390A/V393G/ | | | | | | | | |
| | | V534I/F555G/M558W/M681G/ | | | | | | | | |
| | | K732W/L749F/L750D/I775G/Q921R/ | | | | | | | | |
| | | T1148N/L1248D/L1377D/V1439E | | | | | | | | |
| | | V63Q/ | | | | | | | | |
| 2049/2050 | G619E/G681N/ | L171P/H212A/S260T/E306G/ | ++ | ++ | ++ | ++ | + | ++ | | + |
| | S916T | M320R/S332P/F387I/I390A/V393G/ | | | | | | | | |
| | | V534I/M558W/G619E/M681N/ | | | | | | | | |
| | | K732W/L749F/L750D/I775G/S916T/ | | | | | | | | |
| | | T1148N/L1248D/L1377D/V1439E | | | | | | | | |
| | | V63Q/ | | | | | | | | |
| 2051/2052 | F555G/G619E/ | L171P/H212A/S260T/E306G/ | + | ++ | ++ | ++ | + | ++ | + | + |
| | G681N/K799S | M320R/S332P/F387I/I390A/V393G/ | | | | | | | | |
| | | V534I/F555G/M558W/G619E/M681N/ | | | | | | | | |
| | | K732W/L749F/L750D/I775G/ | | | | | | | | |
| | | K799S/T1148N/L1248D/L1377D/ | | | | | | | | |
| | | V1439E | | | | | | | | |
| 2053/2054 | F555E | V63Q/L171P/H212A/S260T/E306G/ | + | + | + | | | + | | |
| | | M320R/S332P/F387I/I390A/V393G/ | | | | | | | | |
| | | V534I/F555E/M558W/M681G/ | | | | | | | | |
| | | K732W/L749F/L750D/I775G/T1148N/ | | | | | | | | |
| | | L1248D/L1377D/V1439E | | | | | | | | |
| 2055/2056 | W732K | V63Q/L171P/H212A/S260T/E306G/ | | + | + | | | + | | |
| | | M320R/S332P/F387I/I390A/V393G/ | | | | | | | | |
| | | V534I/M558W/M681G/L749F/ | | | | | | | | |
| | | L750D/I775G/T1148N/L1248D/L1377D/ | | | | | | | | |
| | | V1439E | | | | | | | | |
| 2057/2058 | G619E/ | V63Q/L171P/H212A/S260T/E306G/ | ++ | ++ | ++ | | ++ | + | + | + |
| | K799S | M320R/S332P/F387I/I390A/V393G/ | | | | | | | | |
| | | V534I/M558W/G619E/M681G/ | | | | | | | | |
| | | K732W/L749F/L750D/I775G/K799S/ | | | | | | | | |
| | | T1148N/L1248D/L1377D/V1439E | | | | | | | | |
| 2059/2060 | G619E | V63Q/L171P/H212A/S260T/E306G/ | + | + | | + | ++ | ++ | | |
| | | M320R/S332P/F387I/I390A/V393G/ | | | | | | | | |
| | | V534I/M558W/G619E/M681G/ | | | | | | | | |
| | | K732W/L749F/L750D/I775G/T1148N/ | | | | | | | | |
| | | L1248D/L1377D/V1439E | | | | | | | | |
| 2061/2062 | G681N | V63Q/L171P/H212A/S260T/E306G/ | + | ++ | ++ | ++ | + | + | + | |
| | | M320R/S332P/F387I/I390A/V393G/ | | | | | | | | |
| | | V534I/M558W/M681N/K732W/ | | | | | | | | |
| | | L749F/L750D/I775G/T1148N/L1248D/ | | | | | | | | |
| | | L1377D/V1439E | | | | | | | | |
| 2063/2064 | F555G/S916T | V63Q/L171P/H212A/S260T/E306G/ | + | + | ++ | + | | + | + | |
| | | M320R/S332P/F387I/I390A/V393G/ | | | | | | | | |
| | | V534I/F555G/M558W/M681G/ | | | | | | | | |
| | | K732W/L749F/L750D/I775G/S916T/ | | | | | | | | |
| | | T1148N/L1248D/L1377D/V1439E | | | | | | | | |
| 2065/2066 | S839K | V63Q/L171P/H212A/S260T/E306G/ | + | + | + | | ++ | | | + |
| | | M320R/S332P/F387I/I390A/V393G/ | | | | | | | | |
| | | V534I/M558W/M681G/K732W/ | | | | | | | | |
| | | L749F/L750D/I775G/S839K/T1148N/ | | | | | | | | |
| | | L1248D/L1377D/V1439E | | | | | | | | |
| 2067/2068 | K799Q | V63Q/L171P/H212A/S260T/E306G/ | + | + | + | ++ | + | + | | + |
| | | M320R/S332P/F387I/I390A/V393G/ | | | | | | | | |
| | | V534I/M558W/M681G/K732W/ | | | | | | | | |
| | | L749F/L750D/I775G/K799Q/T1148N/ | | | | | | | | |
| | | L1248D/L1377D/V1439E | | | | | | | | |

All activities were determined relative to the reference polypeptide of SEQ ID NO: 1514. Levels of increased activity are defined as follows:
“+” 0.9 to 1.1,
“++” >1.1,
“+++” >2,
“++++” >2.5

### Example 11

### FVIII Variants Derived from SEQ ID NO: 2072

In this Example, experiments conducted to determine FVIII variant activity by assaying the protein activity after a challenge is described. These variants were tested for FVIII activity after no incubation and after incubation in Hemophilia A patient plasma (George King Bio-Medical, Inc.), as described in Example 3. Variants were tested using the chromogenic and clotting assays, as described in Example 3. Results are shown in Table 11.1 for variants expressed using Expi293F cells or HepG2 cells.

TABLE 11.1
Activity of FVIII Variants Relative to SEQ ID NO: 2072

| | | | FIOP | FIOP inv | FIOP | FIOP | FIOP inv | FIOP |
|---|---|---|---|---|---|---|---|---|
| | | | Unchallenged | Unchallenged | Challenged | Unchallenged | Unchallenged | Challenged |
| | Amino Acid | | Biophen | APTT | Biophen | Biophen | APTT | Biophen |
| | Differences | Amino Acid | (Expi) | (Expi) | (Expi) | (HepG2) | (HepG2) | (HepG2) |
| | (Relative to | Differences | Relative to | Relative to | Relative to | Relative to | Relative to | Relative to |
| SEQ ID | SEQ ID NO: | (Relative to | SEQ ID NO | SEQ ID NO | SEQ ID NO | SEQ ID NO | SEQ ID NO | SEQ ID NO |
| NO: (nt/aa) | 2072) | SEQ ID NO: 2) | 2072 | 2072 | 2072 | 2072 | 2072 | 2072 |
| 2073/2074 | A167G | V63Q/A167G/L171P/H212A/S260T/ | +++ | ++ | + | + | + | |
| | | E306G/L318G/M320R/S332P/ | | | | | | |
| | | F387I/I390A/V393G/V534I/M558W/ | | | | | | |
| | | M681G/K732W/Y738E/L749F/ | | | | | | |
| | | L750D/I775G/T1148N/L1248D/ | | | | | | |
| | | L1377D/V1439E | | | | | | |
| 2075/2076 | A167G/E738Y/ | V63Q/A167G/L171P/H212A/S260T/ | ++ | ++ | + | + | + | ++ |
| | E1076S/K1093G | E306G/L318G/M320R/S332P/ | | | | | | |
| | | F387I/I390A/V393G/V534I/M558W/ | | | | | | |
| | | M681G/K732W/L749F/L750D/ | | | | | | |
| | | I775G/E1076S/K1093G/T1148N/ | | | | | | |
| | | L1248D/L1377D/V1439E | | | | | | |
| 2077/2078 | A167G/E738Y/ | V63Q/A167G/L171P/H212A/S260T/ | ++ | ++ | + | + | + | ++ |
| | K752E/E800V/Y805Q/ | E306G/L318G/M320R/S332P/ | | | | | | |
| | D808S/E809G/ | F387I/I390A/V393G/V534I/M558W/ | | | | | | |
| | S1116I | M681G/K732W/L749F/L750D/ | | | | | | |
| | | K752E/I775G/E800V/Y805Q/ | | | | | | |
| | | D808S/E809G/S1116I/T1148N/L1248D/ | | | | | | |
| | | L1377D/V1439E | | | | | | |
| 2079/2080 | A167G/E800V/ | V63Q/A167G/L171P/H212A/S260T/ | ++ | ++ | + | + | + | + |
| | E809G/E1076S | E306G/L318G/M320R/S332P/ | | | | | | |
| | | F387I/I390A/V393G/V534I/M558W/ | | | | | | |
| | | M681G/K732W/Y738E/L749F/ | | | | | | |
| | | L750D/I775G/E800V/E809G/E1076S/ | | | | | | |
| | | T1148N/L1248D/L1377D/ | | | | | | |
| | | V1439E | | | | | | |
| 2081/2082 | A167G/G318L | V63Q/A167G/L171P/H212A/S260T/ | +++ | + | ++ | ++ | ++ | ++ |
| | | E306G/M320R/S332P/F387I/I390A/ | | | | | | |
| | | V393G/V534I/M558W/M681G/ | | | | | | |
| | | K732W/Y738E/L749F/L750D/ | | | | | | |
| | | I775G/T1148N/L1248D/L1377D/ | | | | | | |
| | | V1439E | | | | | | |
| 2083/2084 | A167G/G318L/ | V63Q/A167G/L171P/H212A/S260T/ | +++ | ++ | + | ++ | ++ | ++ |
| | E800V/Y805Q/ | E306G/M320R/S332P/F387I/I390A/ | | | | | | |
| | D808S/E809G | V393G/V534I/M558W/M681G/ | | | | | | |
| | | K732W/Y738E/L749F/L750D/ | | | | | | |
| | | I775G/E800V/Y805Q/D808S/E809G/ | | | | | | |
| | | T1148N/L1248D/L1377D/ | | | | | | |
| | | V1439E | | | | | | |
| 2085/2086 | A167G/G318L/ | V63Q/A167G/L171P/H212A/S260T/ | ++ | + | + | ++ | ++ | ++ |
| | G681N | E306G/M320R/S332P/F387I/I390A/ | | | | | | |
| | | V393G/V534I/M558W/M681N/ | | | | | | |
| | | K732W/Y738E/L749F/L750D/ | | | | | | |
| | | I775G/T1148N/L1248D/L1377D/ | | | | | | |
| | | V1439E | | | | | | |
| 2087/2088 | A167G/G318L/ | V63Q/A167G/L171P/H212A/S260T/ | ++ | ++ | + | ++ | ++ | ++ |
| | G681N/S700E/ | E306G/M320R/S332P/F387I/I390A/ | | | | | | |
| | Y805Q/D808S/ | V393G/V534I/M558W/M681N/ | | | | | | |
| | E809G/E1076S/ | S700E/K732W/Y738E/L749F/ | | | | | | |
| | S1116I | L750D/I775G/Y805Q/D808S/E809G/ | | | | | | |
| | | E1076S/S1116I/T1148N/L1248D/ | | | | | | |
| | | L1377D/V1439E | | | | | | |
| 2089/2090 | A167G/G318L/ | V63Q/A167G/L171P/H212A/S260T/ | ++ | ++ | + | ++ | ++ | ++ |
| | S700E | E306G/M320R/S332P/F387I/I390A/ | | | | | | |
| | | V393G/V534I/M558W/M681G/ | | | | | | |
| | | S700E/K732W/Y738E/L749F/ | | | | | | |
| | | L750D/I775G/T1148N/L1248D/ | | | | | | |
| | | L1377D/V1439E | | | | | | |
| 2091/2092 | A167G/G318L/ | V63Q/A167G/L171P/H212A/S260T/ | ++ | + | ++ | ++ | ++ | ++ |
| | S700E/E1076S | E306G/M320R/S332P/F387I/I390A/ | | | | | | |
| | | V393G/V534I/M558W/M681G/ | | | | | | |
| | | S700E/K732W/Y738E/L749F/ | | | | | | |
| | | L750D/I775G/E1076S/T1148N/ | | | | | | |
| | | L1248D/L1377D/V1439E | | | | | | |
| 2093/2094 | A167G/G318L/ | V63Q/A167G/L171P/H212A/S260T/ | ++ | + | + | ++ | ++ | ++ |
| | S700E/E738Y/ | E306G/M320R/S332P/F387I/I390A/ | | | | | | |
| | E739G/E1076S | V393G/V534I/M558W/M681G/ | | | | | | |
| | | S700E/K732W/E739G/L749F/ | | | | | | |
| | | L750D/I775G/E1076S/T1148N/ | | | | | | |
| | | L1248D/L1377D/V1439E | | | | | | |
| 2095/2096 | A167G/G681N/ | V63Q/A167G/L171P/H212A/S260T/ | ++ | ++ | + | + | + | ++ |
| | E738Y/E800V/ | E306G/L318G/M320R/S332P/ | | | | | | |
| | D808S/E809G | F387I/I390A/V393G/V534I/M558W/ | | | | | | |
| | | M681N/K732W/L749F/L750D/ | | | | | | |
| | | I775G/E800V/D808S/E809G/T1148N/ | | | | | | |
| | | L1248D/L1377D/V1439E | | | | | | |
| 2097/2098 | A167G/L178Q/ | V63Q/A167G/L171P/L178Q/H212A/ | ++ | ++ | + | ++ | ++ | ++ |
| | S700E/K752E/ | S260T/E306G/L318G/M320R/ | | | | | | |
| | K1093G | S332P/F387I/I390A/V393G/V534I/ | | | | | | |
| | | M558W/M681G/S700E/K732W/ | | | | | | |
| | | Y738E/L749F/L750D/K752E/I775G/ | | | | | | |
| | | K1093G/T1148N/L1248D/ | | | | | | |
| | | L1377D/V1439E | | | | | | |
| 2099/2100 | A167G/S700E | V63Q/A167G/L171P/H212A/S260T/ | +++ | + | + | + | + | ++ |
| | | E306G/L318G/M320R/S332P/ | | | | | | |
| | | F387I/I390A/V393G/V534I/M558W/ | | | | | | |
| | | M681G/S700E/K732W/Y738E/ | | | | | | |
| | | L749F/L750D/I775G/T1148N/ | | | | | | |
| | | L1248D/L1377D/V1439E | | | | | | |
| 2101/2102 | A167G/S700E/ | V63Q/A167G/L171P/H212A/S260T/ | + | ++ | + | + | + | ++ |
| | E738Y/E739G/ | E306G/L318G/M320R/S332P/ | | | | | | |
| | K1093G | F387I/I390A/V393G/V534I/M558W/ | | | | | | |
| | | M681G/S700E/K732W/E739G/ | | | | | | |
| | | L749F/L750D/I775G/K1093G/ | | | | | | |
| | | T1148N/L1248D/L1377D/V1439E | | | | | | |
| 2103/2104 | A167G/S700E/ | V63Q/A167G/L171P/H212A/S260T/ | + | ++ | + | + | ++ | +++ |
| | E738Y/K752E/ | E306G/L318G/M320R/S332P/ | | | | | | |
| | K1093G | F387I/I390A/V393G/V534I/M558W/ | | | | | | |
| | | M681G/S700E/K732W/L749F/ | | | | | | |
| | | L750D/K752E/I775G/K1093G/ | | | | | | |
| | | T1148N/L1248D/L1377D/V1439E | | | | | | |
| 2105/2106 | A167G/S700E/ | V63Q/A167G/L171P/H212A/S260T/ | + | ++ | + | + | + | ++ |
| | E738Y/K752E/ | E306G/L318G/M320R/S332P/ | | | | | | |
| | Y805Q/D808S/ | F387I/I390A/V393G/V534I/M558W/ | | | | | | |
| | E809G/K1093A/ | M681G/S700E/K732W/L749F/ | | | | | | |
| | S1116I | L750D/K752E/I775G/Y805Q/D808S/ | | | | | | |
| | | E809G/K1093A/S1116I/T1148N/ | | | | | | |
| | | L1248D/L1377D/V1439E | | | | | | |
| 2107/2108 | D808S/E809G | V63Q/L171P/H212A/S260T/E306G/ | ++ | ++ | ++ | + | + | ++ |
| | | L318G/M320R/S332P/F387I/I390A/ | | | | | | |
| | | V393G/V534I/M558W/M681G/ | | | | | | |
| | | K732W/Y738E/L749F/L750D/ | | | | | | |
| | | I775G/D808S/E809G/T1148N/ | | | | | | |
| | | L1248D/L1377D/V1439E | | | | | | |
| 2109/2110 | E1076S | V63Q/L171P/H212A/S260T/E306G/ | + | + | + | + | + | + |
| | | L318G/M320R/S332P/F387I/I390A/ | | | | | | |
| | | V393G/V534I/M558W/M681G/ | | | | | | |
| | | K732W/Y738E/L749F/L750D/ | | | | | | |
| | | I775G/E1076S/T1148N/L1248D/ | | | | | | |
| | | L1377D/V1439E | | | | | | |
| 2111/2112 | E738Y | V63Q/L171P/H212A/S260T/E306G/ | ++ | ++ | + | ++ | + | ++ |
| | | L318G/M320R/S332P/F387I/I390A/ | | | | | | |
| | | V393G/V534I/M558W/M681G/ | | | | | | |
| | | K732W/L749F/L750D/I775G/ | | | | | | |
| | | T1148N/L1248D/L1377D/V1439E | | | | | | |
| 2113/2114 | E738Y/D808S/ | V63Q/L171P/H212A/S260T/E306G/ | ++ | ++ | + | ++ | + | ++ |
| | E809G | L318G/M320R/S332P/F387I/I390A/ | | | | | | |
| | | V393G/V534I/M558W/M681G/ | | | | | | |
| | | K732W/L749F/L750D/I775G/ | | | | | | |
| | | D808S/E809G/T1148N/L1248D/ | | | | | | |
| | | L1377D/V1439E | | | | | | |
| 2115/2116 | E738Y/E1076S | V63Q/L171P/H212A/S260T/E306G/ | ++ | + | + | + | + | ++ |
| | | L318G/M320R/S332P/F387I/I390A/ | | | | | | |
| | | V393G/V534I/M558W/M681G/ | | | | | | |
| | | K732W/L749F/L750D/I775G/ | | | | | | |
| | | E1076S/T1148N/L1248D/L1377D/ | | | | | | |
| | | V1439E | | | | | | |
| 2117/2118 | E738Y/E739G | V63Q/L171P/H212A/S260T/E306G/ | ++ | + | + | ++ | + | + |
| | | L318G/M320R/S332P/F387I/I390A/ | | | | | | |
| | | V393G/V534I/M558W/M681G/ | | | | | | |
| | | K732W/E739G/L749F/L750D/ | | | | | | |
| | | I775G/T1148N/L1248D/L1377D/ | | | | | | |
| | | V1439E | | | | | | |
| 2119/2120 | E738Y/E739G/ | V63Q/L171P/H212A/S260T/E306G/ | ++ | + | + | ++ | ++ | + |
| | E809G | L318G/M320R/S332P/F387I/I390A/ | | | | | | |
| | | V393G/V534I/M558W/M681G/ | | | | | | |
| | | K732W/E739G/L749F/L750D/ | | | | | | |
| | | I775G/E809G/T1148N/L1248D/ | | | | | | |
| | | L1377D/V1439E | | | | | | |
| 2121/2122 | E738Y/E739G/ | V63Q/L171P/H212A/S260T/E306G/ | + | + | + | ++ | ++ | ++ |
| | E809G/K1093G/ | L318G/M320R/S332P/F387I/I390A/ | | | | | | |
| | S1116I | V393G/V534I/M558W/M681G/ | | | | | | |
| | | K732W/E739G/L749F/L750D/ | | | | | | |
| | | I775G/E809G/K1093G/S1116I/ | | | | | | |
| | | T1148N/L1248D/L1377D/V1439E | | | | | | |
| 2123/2124 | E738Y/E739G/ | V63Q/L171P/H212A/S260T/E306G/ | +++ | ++ | + | ++ | + | ++ |
| | K1093G | L318G/M320R/S332P/F387I/I390A/ | | | | | | |
| | | V393G/V534I/M558W/M681G/ | | | | | | |
| | | K732W/E739G/L749F/L750D/ | | | | | | |
| | | I775G/K1093G/T1148N/L1248D/ | | | | | | |
| | | L1377D/V1439E | | | | | | |
| 2125/2126 | E738Y/E739G/ | V63Q/L171P/H212A/S260T/E306G/ | ++ | ++ | ++ | ++ | ++ | ++ |
| | K752E/K1093G | L318G/M320R/S332P/F387I/I390A/ | | | | | | |
| | | V393G/V534I/M558W/M681G/ | | | | | | |
| | | K732W/E739G/L749F/L750D/ | | | | | | |
| | | K752E/I775G/K1093G/T1148N/ | | | | | | |
| | | L1248D/L1377D/V1439E | | | | | | |
| 2127/2128 | E738Y/E739G/ | V63Q/L171P/H212A/S260T/E306G/ | ++ | + | + | ++ | ++ | |
| | K799S/E800V/ | L318G/M320R/S332P/F387I/I390A/ | | | | | | |
| | K1093G | V393G/V534I/M558W/M681G/ | | | | | | |
| | | K732W/E739G/L749F/L750D/ | | | | | | |
| | | I775G/K799S/E800V/K1093G/ | | | | | | |
| | | T1148N/L1248D/L1377D/V1439E | | | | | | |
| 2129/2130 | E738Y/E739G/ | V63Q/L171P/H212A/S260T/E306G/ | ++ | + | + | + | + | |
| | S1116I | L318G/M320R/S332P/F387I/I390A/ | | | | | | |
| | | V393G/V534I/M558W/M681G/ | | | | | | |
| | | K732W/E739G/L749F/L750D/ | | | | | | |
| | | I775G/S1116I/T1148N/L1248D/ | | | | | | |
| | | L1377D/V1439E | | | | | | |
| 2131/2132 | E738Y/E800V | V63Q/L171P/H212A/S260T/E306G/ | ++ | + | + | + | + | |
| | | L318G/M320R/S332P/F387I/I390A/ | | | | | | |
| | | V393G/V534I/M558W/M681G/ | | | | | | |
| | | K732W/L749F/L750D/I775G/ | | | | | | |
| | | E800V/T1148N/L1248D/L1377D/ | | | | | | |
| | | V1439E | | | | | | |
| 2133/2134 | E738Y/K1093G | V63Q/L171P/H212A/S260T/E306G/ | ++ | ++ | + | ++ | ++ | |
| | | L318G/M320R/S332P/F387I/I390A/ | | | | | | |
| | | V393G/V534I/M558W/M681G/ | | | | | | |
| | | K732W/L749F/L750D/I775G/ | | | | | | |
| | | K1093G/T1148N/L1248D/L1377D/ | | | | | | |
| | | V1439E | | | | | | |
| 2135/2136 | E738Y/K752E | V63Q/L171P/H212A/S260T/E306G/ | + | ++ | + | ++ | ++ | ++ |
| | | L318G/M320R/S332P/F387I/I390A/ | | | | | | |
| | | V393G/V534I/M558W/M681G/ | | | | | | |
| | | K732W/L749F/L750D/K752E/ | | | | | | |
| | | I775G/T1148N/L1248D/L1377D/ | | | | | | |
| | | V1439E | | | | | | |
| 2137/2138 | E738Y/K752E/ | V63Q/L171P/H212A/S260T/E306G/ | +++ | ++ | + | ++ | ++ | |
| | K1093G | L318G/M320R/S332P/F387I/I390A/ | | | | | | |
| | | V393G/V534I/M558W/M681G/ | | | | | | |
| | | K732W/L749F/L750D/K752E/ | | | | | | |
| | | I775G/K1093G/T1148N/L1248D/ | | | | | | |
| | | L1377D/V1439E | | | | | | |
| 2139/2140 | E738Y/K752E/ | V63Q/L171P/H212A/S260T/E306G/ | ++ | + | + | + | + | |
| | K799S | L318G/M320R/S332P/F387I/I390A/ | | | | | | |
| | | V393G/V534I/M558W/M681G/ | | | | | | |
| | | K732W/L749F/L750D/K752E/ | | | | | | |
| | | I775G/K799S/T1148N/L1248D/ | | | | | | |
| | | L1377D/V1439E | | | | | | |
| 2141/2142 | E738Y/K799S/ | V63Q/L171P/H212A/S260T/E306G/ | + | + | + | ++ | ++ | ++ |
| | E809G/K1093G | L318G/M320R/S332P/F387I/I390A/ | | | | | | |
| | | V393G/V534I/M558W/M681G/ | | | | | | |
| | | K732W/L749F/L750D/I775G/ | | | | | | |
| | | K799S/E809G/K1093G/T1148N/ | | | | | | |
| | | L1248D/L1377D/V1439E | | | | | | |
| 2143/2144 | E738Y/K799S/ | V63Q/L171P/H212A/S260T/E306G/ | ++ | + | ++ | + | ++ | ++ |
| | K1093G | L318G/M320R/S332P/F387I/I390A/ | | | | | | |
| | | V393G/V534I/M558W/M681G/ | | | | | | |
| | | K732W/L749F/L750D/I775G/ | | | | | | |
| | | K799S/K1093G/T1148N/L1248D/ | | | | | | |
| | | L1377D/V1439E | | | | | | |
| 2145/2146 | E739G | V63Q/L171P/H212A/S260T/E306G/ | ++ | + | + | + | + | ++ |
| | | L318G/M320R/S332P/F387I/I390A/ | | | | | | |
| | | V393G/V534I/M558W/M681G/ | | | | | | |
| | | K732W/Y738E/E739G/L749F/ | | | | | | |
| | | L750D/I775G/T1148N/L1248D/ | | | | | | |
| | | L1377D/V1439E | | | | | | |
| 2147/2148 | E739G/E800V/ | V63Q/L171P/H212A/S260T/E306G/ | ++ | + | + | + | + | + |
| | E809G | L318G/M320R/S332P/F387I/I390A/ | | | | | | |
| | | V393G/V534I/M558W/M681G/ | | | | | | |
| | | K732W/Y738E/E739G/L749F/ | | | | | | |
| | | L750D/I775G/E800V/E809G/T1148N/ | | | | | | |
| | | L1248D/L1377D/V1439E | | | | | | |
| 2149/2150 | E800V | V63Q/L171P/H212A/S260T/E306G/ | ++ | + | + | + | + | |
| | | L318G/M320R/S332P/F387I/I390A/ | | | | | | |
| | | V393G/V534I/M558W/M681G/ | | | | | | |
| | | K732W/Y738E/L749F/L750D/ | | | | | | |
| | | I775G/E800V/T1148N/L1248D/ | | | | | | |
| | | L1377D/V1439E | | | | | | |
| 2151/2152 | E800V/Y805Q/ | V63Q/L171P/H212A/S260T/E306G/ | ++ | ++ | + | + | ++ | ++ |
| | E809G | L318G/M320R/S332P/F387I/I390A/ | | | | | | |
| | | V393G/V534I/M558W/M681G/ | | | | | | |
| | | K732W/Y738E/L749F/L750D/ | | | | | | |
| | | I775G/E800V/Y805Q/E809G/ | | | | | | |
| | | T1148N/L1248D/L1377D/V1439E | | | | | | |
| 2153/2154 | E809G | V63Q/L171P/H212A/S260T/E306G/ | ++++ | + | + | ++ | + | + |
| | | L318G/M320R/S332P/F387I/I390A/ | | | | | | |
| | | V393G/V534I/M558W/M681G/ | | | | | | |
| | | K732W/Y738E/L749F/L750D/ | | | | | | |
| | | I775G/E809G/T1148N/L1248D/ | | | | | | |
| | | L1377D/V1439E | | | | | | |
| 2155/2156 | E809G/K1093G | V63Q/L171P/H212A/S260T/E306G/ | ++ | + | ++ | ++ | + | |
| | | L318G/M320R/S332P/F387I/I390A/ | | | | | | |
| | | V393G/V534I/M558W/M681G/ | | | | | | |
| | | K732W/Y738E/L749F/L750D/ | | | | | | |
| | | I775G/E809G/K1093G/T1148N/ | | | | | | |
| | | L1248D/L1377D/V1439E | | | | | | |
| 2157/2158 | E809G/K1093G/ | V63Q/L171P/H212A/S260T/E306G/ | ++ | + | ++ | + | + | ++ |
| | S1116I | L318G/M320R/S332P/F387I/I390A/ | | | | | | |
| | | V393G/V534I/M558W/M681G/ | | | | | | |
| | | K732W/Y738E/L749F/L750D/ | | | | | | |
| | | I775G/E809G/K1093G/S1116I/ | | | | | | |
| | | T1148N/L1248D/L1377D/V1439E | | | | | | |
| 2159/2160 | E809G/S1116I | V63Q/L171P/H212A/S260T/E306G/ | ++ | + | + | ++ | + | ++ |
| | | L318G/M320R/S332P/F387I/I390A/ | | | | | | |
| | | V393G/V534I/M558W/M681G/ | | | | | | |
| | | K732W/Y738E/L749F/L750D/ | | | | | | |
| | | I775G/E809G/S1116I/T1148N/ | | | | | | |
| | | L1248D/L1377D/V1439E | | | | | | |
| 2161/2162 | G318L | V63Q/L171P/H212A/S260T/E306G/ | +++ | + | ++ | ++ | ++ | ++ |
| | | M320R/S332P/F387I/I390A/ | | | | | | |
| | | V393G/V534I/M558W/M681G/K732W/ | | | | | | |
| | | Y738E/L749F/L750D/I775G/ | | | | | | |
| | | T1148N/L1248D/L1377D/V1439E | | | | | | |
| 2163/2164 | G318L/E1076S | V63Q/L171P/H212A/S260T/E306G/ | +++ | ++ | + | ++ | ++ | ++ |
| | | M320R/S332P/F387I/I390A/ | | | | | | |
| | | V393G/V534I/M558W/M681G/K732W/ | | | | | | |
| | | Y738E/L749F/L750D/I775G/ | | | | | | |
| | | E1076S/T1148N/L1248D/L1377D/ | | | | | | |
| | | V1439E | | | | | | |
| 2165/2166 | G318L/E1076S/ | V63Q/L171P/H212A/S260T/E306G/ | ++ | + | + | ++ | ++ | ++ |
| | S1116I | M320R/S332P/F387I/I390A/ | | | | | | |
| | | V393G/V534I/M558W/M681G/K732W/ | | | | | | |
| | | Y738E/L749F/L750D/I775G/ | | | | | | |
| | | E1076S/S1116I/T1148N/L1248D/ | | | | | | |
| | | L1377D/V1439E | | | | | | |
| 2167/2168 | G318L/E739G/ | V63Q/L171P/H212A/S260T/E306G/ | ++ | ++ | ++ | ++ | ++ | ++ |
| | Y805Q/D808S/ | M320R/S332P/F387I/I390A/ | | | | | | |
| | E809G/E1076S | V393G/V534I/M558W/M681G/K732W/ | | | | | | |
| | | Y738E/E739G/L749F/L750D/ | | | | | | |
| | | I775G/Y805Q/D808S/E809G/ | | | | | | |
| | | E1076S/T1148N/L1248D/L1377D/ | | | | | | |
| | | V1439E | | | | | | |
| 2169/2170 | G318L/I499L/S700E/ | V63Q/L171P/H212A/S260T/E306G/ | ++++ | ++ | + | ++ | ++ | +++ |
| | E738Y/E809G | M320R/S332P/F387I/I390A/ | | | | | | |
| | | V393G/I499L/V534I/M558W/M681G/ | | | | | | |
| | | S700E/K732W/L749F/L750D/ | | | | | | |
| | | I775G/E809G/T1148N/L1248D/ | | | | | | |
| | | L1377D/V1439E | | | | | | |
| 2171/2172 | G318L/K1093A | V63Q/L171P/H212A/S260T/E306G/ | ++ | ++ | ++ | ++ | ++ | ++ |
| | | M320R/S332P/F387I/I390A/ | | | | | | |
| | | V393G/V534I/M558W/M681G/K732W/ | | | | | | |
| | | Y738E/L749F/L750D/I775G/ | | | | | | |
| | | K1093A/T1148N/L1248D/L1377D/ | | | | | | |
| | | V1439E | | | | | | |
| 2173/2174 | G318L/K752E/ | V63Q/L171P/H212A/S260T/E306G/ | ++ | ++ | ++ | ++ | ++ | +++ |
| | K1093A | M320R/S332P/F387I/I390A/ | | | | | | |
| | | V393G/V534I/M558W/M681G/K732W/ | | | | | | |
| | | Y738E/L749F/L750D/K752E/ | | | | | | |
| | | I775G/K1093A/T1148N/L1248D/ | | | | | | |
| | | L1377D/V1439E | | | | | | |
| 2175/2176 | G318L/S1116I | V63Q/L171P/H212A/S260T/E306G/ | ++ | ++ | + | ++ | ++ | ++ |
| | | M320R/S332P/F387I/I390A/ | | | | | | |
| | | V393G/V534I/M558W/M681G/K732W/ | | | | | | |
| | | Y738E/L749F/L750D/I775G/ | | | | | | |
| | | S1116I/T1148N/L1248D/L1377D/ | | | | | | |
| | | V1439E | | | | | | |
| 2177/2178 | G318L/S543P/ | V63Q/L171P/H212A/S260T/E306G/ | ++ | + | + | ++ | ++ | ++ |
| | E739G | M320R/S332P/F387I/I390A/ | | | | | | |
| | | V393G/V534I/S543P/M558W/M681G/ | | | | | | |
| | | K732W/Y738E/E739G/L749F/ | | | | | | |
| | | L750D/I775G/T1148N/L1248D/ | | | | | | |
| | | L1377D/V1439E | | | | | | |
| 2179/2180 | G318L/S543P/ | V63Q/L171P/H212A/S260T/E306G/ | ++ | ++ | + | ++ | ++ | +++ |
| | G68IN/K752E/ | M320R/S332P/F387I/I390A/ | | | | | | |
| | S1116I | V393G/V534I/S543P/M558W/M681N/ | | | | | | |
| | | K732W/Y738E/L749F/L750D/ | | | | | | |
| | | K752E/I775G/S1116I/T1148N/ | | | | | | |
| | | L1248D/L1377D/V1439E | | | | | | |
| 2181/2182 | G318L/S543P/ | V63Q/L171P/H212A/S260T/E306G/ | ++ | + | + | ++ | ++ | ++ |
| | K1093A | M320R/S332P/F387I/I390A/ | | | | | | |
| | | V393G/V534I/S543P/M558W/M681G/ | | | | | | |
| | | K732W/Y738E/L749F/L750D/ | | | | | | |
| | | I775G/K1093A/T1148N/L1248D/ | | | | | | |
| | | L1377D/V1439E | | | | | | |
| 2183/2184 | G318L/S700E/E738Y/ | V63Q/L171P/H212A/S260T/E306G/ | +++ | ++ | + | ++ | ++ | ++++ |
| | K1093A/S1414N | M320R/S332P/F387I/I390A/ | | | | | | |
| | | V393G/V534I/M558W/M681G/S700E/ | | | | | | |
| | | K732W/L749F/L750D/I775G/ | | | | | | |
| | | K1093A/T1148N/L1248D/L1377D/ | | | | | | |
| | | S1414N/V1439E | | | | | | |
| 2185/2186 | G681N | V63Q/L171P/H212A/S260T/E306G/ | ++ | ++ | + | ++ | + | ++ |
| | | L318G/M320R/S332P/F387I/I390A/ | | | | | | |
| | | V393G/V534I/M558W/M681N/ | | | | | | |
| | | K732W/Y738E/L749F/L750D/ | | | | | | |
| | | I775G/T1148N/L1248D/L1377D/ | | | | | | |
| | | V1439E | | | | | | |
| 2187/2188 | G681N/E738Y | V63Q/L171P/H212A/S260T/E306G/ | ++ | ++ | + | + | + | ++ |
| | | L318G/M320R/S332P/F387I/I390A/ | | | | | | |
| | | V393G/V534I/M558W/M681N/ | | | | | | |
| | | K732W/L749F/L750D/I775G/ | | | | | | |
| | | T1148N/L1248D/L1377D/V1439E | | | | | | |
| 2189/2190 | G681N/E738Y/ | V63Q/L171P/H212A/S260T/E306G/ | ++ | + | ++ | + | ++ | ++ |
| | E739G/K1093G | L318G/M320R/S332P/F387I/I390A/ | | | | | | |
| | | V393G/V534I/M558W/M681N/ | | | | | | |
| | | K732W/E739G/L749F/L750D/ | | | | | | |
| | | I775G/K1093G/T1148N/L1248D/ | | | | | | |
| | | L1377D/V1439E | | | | | | |
| 2191/2192 | G681N/E738Y/E739G/ | V63Q/L171P/H212A/S260T/E306G/ | ++ | ++ | + | + | + | |
| | K752E/K799S/ | L318G/M320R/S332P/F387I/I390A/ | | | | | | |
| | E800V/E809G/ | V393G/V534I/M558W/M681N/ | | | | | | |
| | S1116I/G1339C | K732W/E739G/L749F/L750D/ | | | | | | |
| | | K752E/I775G/K799S/E800V/E809G/ | | | | | | |
| | | S1116I/T1148N/L1248D/G1339C/ | | | | | | |
| | | L1377D/V1439E | | | | | | |
| 2193/2194 | G681N/E738Y/ | V63Q/L171P/H212A/S260T/E306G/ | +++ | ++ | + | ++ | + | ++ |
| | E739G/S1116I | L318G/M320R/S332P/F387I/I390A/ | | | | | | |
| | | V393G/V534I/M558W/M681N/ | | | | | | |
| | | K732W/E739G/L749F/L750D/ | | | | | | |
| | | I775G/S1116I/T1148N/L1248D/ | | | | | | |
| | | L1377D/V1439E | | | | | | |
| 2195/2196 | G681N/E738Y/ | V63Q/L171P/H212A/S260T/E306G/ | ++ | ++ | + | ++ | + | |
| | E800V | L318G/M320R/S332P/F387I/I390A/ | | | | | | |
| | | V393G/V534I/M558W/M681N/ | | | | | | |
| | | K732W/L749F/L750D/I775G/ | | | | | | |
| | | E800V/T1148N/L1248D/L1377D/ | | | | | | |
| | | V1439E | | | | | | |
| 2197/2198 | G681N/E800V/ | V63Q/L171P/H212A/S260T/E306G/ | ++++ | ++ | + | ++ | ++ | ++ |
| | Y805Q/E809G | L318G/M320R/S332P/F387I/I390A/ | | | | | | |
| | | V393G/V534I/M558W/M681N/ | | | | | | |
| | | K732W/Y738E/L749F/L750D/ | | | | | | |
| | | I775G/E800V/Y805Q/E809G/ | | | | | | |
| | | T1148N/L1248D/L1377D/V1439E | | | | | | |
| 2199/2200 | G681N/E809G/ | V63Q/L171P/H212A/S260T/E306G/ | ++ | ++ | ++ | + | ++ | |
| | K1093G | L318G/M320R/S332P/F387I/I390A/ | | | | | | |
| | | V393G/V534I/M558W/M681N/ | | | | | | |
| | | K732W/Y738E/L749F/L750D/ | | | | | | |
| | | I775G/E809G/K1093G/T1148N/ | | | | | | |
| | | L1248D/L1377D/V1439E | | | | | | |
| 2201/2202 | G681N/K752E/ | V63Q/L171P/H212A/S260T/E306G/ | ++ | ++ | + | + | ++ | +++ |
| | K1093A | L318G/M320R/S332P/F387I/I390A/ | | | | | | |
| | | V393G/V534I/M558W/M681N/ | | | | | | |
| | | K732W/Y738E/L749F/L750D/ | | | | | | |
| | | K752E/I775G/K1093A/T1148N/ | | | | | | |
| | | L1248D/L1377D/V1439E | | | | | | |
| 2203/2204 | G681N/K752E/ | V63Q/L171P/H212A/S260T/E306G/ | ++ | ++ | + | ++ | ++ | +++ |
| | K1093G | L318G/M320R/S332P/F387I/I390A/ | | | | | | |
| | | V393G/V534I/M558W/M681N/ | | | | | | |
| | | K732W/Y738E/L749F/L750D/ | | | | | | |
| | | K752E/I775G/K1093G/T1148N/ | | | | | | |
| | | L1248D/L1377D/V1439E | | | | | | |
| 2205/2206 | G681N/S700E | V63Q/L171P/H212A/S260T/E306G/ | ++ | ++ | + | ++ | ++ | ++ |
| | | L318G/M320R/S332P/F387I/I390A/ | | | | | | |
| | | V393G/V534I/M558W/M681N/ | | | | | | |
| | | S700E/K732W/Y738E/L749F/ | | | | | | |
| | | L750D/I775G/T1148N/L1248D/ | | | | | | |
| | | L1377D/V1439E | | | | | | |
| 2207/2208 | G681N/S700E/ | V63Q/L171P/H212A/S260T/E306G/ | ++ | ++ | + | ++ | + | ++ |
| | E738Y/E739G/ | L318G/M320R/S332P/F387I/I390A/ | | | | | | |
| | K1093G/S1116I | V393G/V534I/M558W/M681N/ | | | | | | |
| | | S700E/K732W/E739G/L749F/ | | | | | | |
| | | L750D/I775G/K1093G/S1116I/ | | | | | | |
| | | T1148N/L1248D/L1377D/V1439E | | | | | | |
| 2209/2210 | G681N/S700E/ | V63Q/L171P/H212A/S260T/E306G/ | + | + | + | ++ | + | ++ |
| | E809G | L318G/M320R/S332P/F387I/I390A/ | | | | | | |
| | | V393G/V534I/M558W/M681N/ | | | | | | |
| | | S700E/K732W/Y738E/L749F/ | | | | | | |
| | | L750D/I775G/E809G/T1148N/ | | | | | | |
| | | L1248D/L1377D/V1439E | | | | | | |
| 2211/2212 | G681N/S700E/ | V63Q/L171P/H212A/S260T/E306G/ | ++ | ++ | + | ++ | ++ | ++ |
| | K1093G | L318G/M320R/S332P/F387I/I390A/ | | | | | | |
| | | V393G/V534I/M558W/M681N/ | | | | | | |
| | | S700E/K732W/Y738E/L749F/ | | | | | | |
| | | L750D/I775G/K1093G/T1148N/ | | | | | | |
| | | L1248D/L1377D/V1439E | | | | | | |
| 2213/2214 | G681N/S700E/ | V63Q/L171P/H212A/S260T/E306G/ | + | ++ | ++ | + | ++ | |
| | K752E/D808S/ | L318G/M320R/S332P/F387I/I390A/ | | | | | | |
| | E809G/K1093G/ | V393G/V534I/M558W/M681N/ | | | | | | |
| | S1116I | S700E/K732W/Y738E/L749F/ | | | | | | |
| | | L750D/K752E/I775G/D808S/E809G/ | | | | | | |
| | | K1093G/S1116I/T1148N/L1248D/ | | | | | | |
| | | L1377D/V1439E | | | | | | |
| 2215/2216 | G681N/S700E/ | V63Q/L171P/H212A/S260T/E306G/ | +++ | ++ | ++ | ++ | ++ | |
| | K752E/K1093G | L318G/M320R/S332P/F387I/I390A/ | | | | | | |
| | | V393G/V534I/M558W/M681N/ | | | | | | |
| | | S700E/K732W/Y738E/L749F/ | | | | | | |
| | | L750D/K752E/I775G/K1093G/ | | | | | | |
| | | T1148N/L1248D/L1377D/V1439E | | | | | | |
| 2217/2218 | G681N/S700E/ | V63Q/L171P/H212A/S260T/E306G/ | + | + | + | + | + | ++ |
| | R844H | L318G/M320R/S332P/F387I/I390A/ | | | | | | |
| | | V393G/V534I/M558W/M681N/ | | | | | | |
| | | S700E/K732W/Y738E/L749F/ | | | | | | |
| | | L750D/I775G/R844H/T1148N/ | | | | | | |
| | | L1248D/L1377D/V1439E | | | | | | |
| 2219/2220 | G681N/S700E/ | V63Q/L171P/H212A/S260T/E306G/ | ++ | ++ | ++ | ++ | + | ++ |
| | Y805Q/E809G | L318G/M320R/S332P/F387I/I390A/ | | | | | | |
| | | V393G/V534I/M558W/M681N/ | | | | | | |
| | | S700E/K732W/Y738E/L749F/ | | | | | | |
| | | L750D/I775G/Y805Q/E809G/T1148N/ | | | | | | |
| | | L1248D/L1377D/V1439E | | | | | | |
| 2221/2222 | K1093A | V63Q/L171P/H212A/S260T/E306G/ | ++ | ++ | + | + | ++ | |
| | | L318G/M320R/S332P/F387I/I390A/ | | | | | | |
| | | V393G/V534I/M558W/M681G/ | | | | | | |
| | | K732W/Y738E/L749F/L750D/ | | | | | | |
| | | I775G/K1093A/T1148N/L1248D/ | | | | | | |
| | | L1377D/V1439E | | | | | | |
| 2223/2224 | K1093G | V63Q/L171P/H212A/S260T/E306G/ | ++ | + | + | ++ | + | + |
| | | L318G/M320R/S332P/F387I/I390A/ | | | | | | |
| | | V393G/V534I/M558W/M681G/ | | | | | | |
| | | K732W/Y738E/L749F/L750D/ | | | | | | |
| | | I775G/K1093G/T1148N/L1248D/ | | | | | | |
| | | L1377D/V1439E | | | | | | |
| 2225/2226 | K1093G/S1116I | V63Q/L171P/H212A/S260T/E306G/ | ++ | ++ | + | ++ | ++ | ++ |
| | | L318G/M320R/S332P/F387I/I390A/ | | | | | | |
| | | V393G/V534I/M558W/M681G/ | | | | | | |
| | | K732W/Y738E/L749F/L750D/ | | | | | | |
| | | I775G/K1093G/S1116I/T1148N/ | | | | | | |
| | | L1248D/L1377D/V1439E | | | | | | |
| 2227/2228 | K752E | V63Q/L171P/H212A/S260T/E306G/ | ++ | + | ++ | ++ | + | |
| | | L318G/M320R/S332P/F387I/I390A/ | | | | | | |
| | | V393G/V534I/M558W/M681G/ | | | | | | |
| | | K732W/Y738E/L749F/L750D/ | | | | | | |
| | | K752E/I775G/T1148N/L1248D/ | | | | | | |
| | | L1377D/V1439E | | | | | | |
| 2229/2230 | K752E/E1076S | V63Q/L171P/H212A/S260T/E306G/ | ++ | ++ | + | + | ++ | ++ |
| | | L318G/M320R/S332P/F387I/I390A/ | | | | | | |
| | | V393G/V534I/M558W/M681G/ | | | | | | |
| | | K732W/Y738E/L749F/L750D/ | | | | | | |
| | | K752E/I775G/E1076S/T1148N/ | | | | | | |
| | | L1248D/L1377D/V1439E | | | | | | |
| 2231/2232 | K752E/E800V/ | V63Q/L171P/H212A/S260T/E306G/ | ++ | ++ | + | + | + | ++ |
| | E809G | L318G/M320R/S332P/F387I/I390A/ | | | | | | |
| | | V393G/V534I/M558W/M681G/ | | | | | | |
| | | K732W/Y738E/L749F/L750D/ | | | | | | |
| | | K752E/I775G/E800V/E809G/T1148N/ | | | | | | |
| | | L1248D/L1377D/V1439E | | | | | | |
| 2233/2234 | K752E/E800V/ | V63Q/L171P/H212A/S260T/E306G/ | ++ | ++ | + | ++ | ++ | ++ |
| | K1093G | L318G/M320R/S332P/F387I/I390A/ | | | | | | |
| | | V393G/V534I/M558W/M681G/ | | | | | | |
| | | K732W/Y738E/L749F/L750D/ | | | | | | |
| | | K752E/I775G/E800V/K1093G/ | | | | | | |
| | | T1148N/L1248D/L1377D/V1439E | | | | | | |
| 2235/2236 | K752E/E809G | V63Q/L171P/H212A/S260T/E306G/ | + | + | + | + | + | ++ |
| | | L318G/M320R/S332P/F387I/I390A/ | | | | | | |
| | | V393G/V534I/M558W/M681G/ | | | | | | |
| | | K732W/Y738E/L749F/L750D/ | | | | | | |
| | | K752E/I775G/E809G/T1148N/ | | | | | | |
| | | L1248D/L1377D/V1439E | | | | | | |
| 2237/2238 | K752E/G866A | V63Q/L171P/H212A/S260T/E306G/ | ++ | ++ | + | + | + | |
| | | L318G/M320R/S332P/F387I/I390A/ | | | | | | |
| | | V393G/V534I/M558W/M681G/ | | | | | | |
| | | K732W/Y738E/L749F/L750D/ | | | | | | |
| | | K752E/I775G/G866A/T1148N/ | | | | | | |
| | | L1248D/L1377D/V1439E | | | | | | |
| 2239/2240 | K752E/K1093G | V63Q/L171P/H212A/S260T/E306G/ | + | ++ | ++ | ++ | ++ | ++ |
| | | L318G/M320R/S332P/F387I/I390A/ | | | | | | |
| | | V393G/V534I/M558W/M681G/ | | | | | | |
| | | K732W/Y738E/L749F/L750D/ | | | | | | |
| | | K752E/I775G/K1093G/T1148N/ | | | | | | |
| | | L1248D/L1377D/V1439E | | | | | | |
| 2241/2242 | K752E/S1116I | V63Q/L171P/H212A/S260T/E306G/ | + | + | + | + | + | ++ |
| | | L318G/M320R/S332P/F387I/I390A/ | | | | | | |
| | | V393G/V534I/M558W/M681G/ | | | | | | |
| | | K732W/Y738E/L749F/L750D/ | | | | | | |
| | | K752E/I775G/S1116I/T1148N/ | | | | | | |
| | | L1248D/L1377D/V1439E | | | | | | |
| 2243/2244 | L178Q/G318L/E739G/ | V63Q/L171P/L178Q/H212A/S260T/ | ++ | ++ | + | ++ | ++ | ++ |
| | K752E/E809G/ | E306G/M320R/S332P/F387I/I390A/ | | | | | | |
| | E1076S/S1116I | V393G/V534I/M558W/M681G/ | | | | | | |
| | | K732W/Y738E/E739G/L749F/ | | | | | | |
| | | L750D/K752E/I775G/E809G/E1076S/ | | | | | | |
| | | S1116I/T1148N/L1248D/L1377D/ | | | | | | |
| | | V1439E | | | | | | |
| 2245/2246 | L178Q/G318L/ | V63Q/L171P/L178Q/H212A/S260T/ | ++ | + | + | ++ | ++ | ++ |
| | E800V/E809G | E306G/M320R/S332P/F387I/I390A/ | | | | | | |
| | | V393G/V534I/M558W/M681G/ | | | | | | |
| | | K732W/Y738E/L749F/L750D/ | | | | | | |
| | | I775G/E800V/E809G/T1148N/ | | | | | | |
| | | L1248D/L1377D/V1439E | | | | | | |
| 2247/2248 | L178Q/G318L/G681N/ | V63Q/L171P/L178Q/H212A/S260T/ | ++ | ++ | + | ++ | ++ | ++ |
| | E800V/D808S/ | E306G/M320R/S332P/F387I/I390A/ | | | | | | |
| | E809G/E1076S | V393G/V534I/M558W/M681N/ | | | | | | |
| | | K732W/Y738E/L749F/L750D/ | | | | | | |
| | | I775G/E800V/D808S/E809G/E1076S/ | | | | | | |
| | | T1148N/L1248D/L1377D/ | | | | | | |
| | | V1439E | | | | | | |
| 2249/2250 | L178Q/G681N | V63Q/L171P/L178Q/H212A/S260T/ | ++ | ++ | + | ++ | ++ | ++ |
| | | E306G/L318G/M320R/S332P/ | | | | | | |
| | | F387I/I390A/V393G/V534I/M558W/ | | | | | | |
| | | M681N/K732W/Y738E/L749F/ | | | | | | |
| | | L750D/I775G/T1148N/L1248D/ | | | | | | |
| | | L1377D/V1439E | | | | | | |
| 2251/2252 | L178Q/G681N/ | V63Q/L171P/L178Q/H212A/S260T/ | ++ | ++ | + | ++ | ++ | ++ |
| | E738Y/S1116I | E306G/L318G/M320R/S332P/ | | | | | | |
| | | F387I/I390A/V393G/V534I/M558W/ | | | | | | |
| | | M681N/K732W/L749F/L750D/ | | | | | | |
| | | I775G/S1116I/T1148N/L1248D/ | | | | | | |
| | | L1377D/V1439E | | | | | | |
| 2253/2254 | L178Q/K752E/ | V63Q/L171P/L178Q/H212A/S260T/ | + | ++ | + | ++ | + | ++ |
| | E809G | E306G/L318G/M320R/S332P/ | | | | | | |
| | | F387I/I390A/V393G/V534I/M558W/ | | | | | | |
| | | M681G/K732W/Y738E/L749F/ | | | | | | |
| | | L750D/K752E/I775G/E809G/T1148N/ | | | | | | |
| | | L1248D/L1377D/V1439E | | | | | | |
| 2255/2256 | L178Q/S700E/E800V/ | V63Q/L171P/L178Q/H212A/S260T/ | ++ | ++ | + | ++ | + | ++ |
| | Y805Q/D808S/ | E306G/L318G/M320R/S332P/ | | | | | | |
| | E809G | F387I/I390A/V393G/V534I/M558W/ | | | | | | |
| | | M681G/S700E/K732W/Y738E/ | | | | | | |
| | | L749F/L750D/I775G/E800V/Y805Q/ | | | | | | |
| | | D808S/E809G/T1148N/L1248D/ | | | | | | |
| | | L1377D/V1439E | | | | | | |
| 2257/2258 | L178Q/Y805Q/ | V63Q/L171P/L178Q/H212A/S260T/ | ++ | + | + | ++ | ++ | + |
| | E809G/G866A | E306G/L318G/M320R/S332P/ | | | | | | |
| | | F387I/I390A/V393G/V534I/M558W/ | | | | | | |
| | | M681G/K732W/Y738E/L749F/ | | | | | | |
| | | L750D/I775G/Y805Q/E809G/G866A/ | | | | | | |
| | | T1148N/L1248D/L1377D/ | | | | | | |
| | | V1439E | | | | | | |
| 2259/2260 | S1116I | V63Q/L171P/H212A/S260T/E306G/ | ++ | + | + | + | + | |
| | | L318G/M320R/S332P/F387I/I390A/ | | | | | | |
| | | V393G/V534I/M558W/M681G/ | | | | | | |
| | | K732W/Y738E/L749F/L750D/ | | | | | | |
| | | I775G/S1116I/T1148N/L1248D/ | | | | | | |
| | | L1377D/V1439E | | | | | | |
| 2261/2262 | S543P | V63Q/L171P/H212A/S260T/E306G/ | ++ | + | + | + | + | + |
| | | L318G/M320R/S332P/F387I/I390A/ | | | | | | |
| | | V393G/V534I/S543P/M558W/ | | | | | | |
| | | M681G/K732W/Y738E/L749F/ | | | | | | |
| | | L750D/I775G/T1148N/L1248D/ | | | | | | |
| | | L1377D/V1439E | | | | | | |
| 2263/2264 | S543P/E738Y/E739G/ | V63Q/L171P/H212A/S260T/E306G/ | ++ | ++ | + | ++ | + | |
| | E809G | L318G/M320R/S332P/F387I/I390A/ | | | | | | |
| | | V393G/V534I/S543P/M558W/ | | | | | | |
| | | M681G/K732W/E739G/L749F/ | | | | | | |
| | | L750D/I775G/E809G/T1148N/L1248D/ | | | | | | |
| | | L1377D/V1439E | | | | | | |
| 2265/2266 | S543P/E738Y/E809G/ | V63Q/L171P/H212A/S260T/E306G/ | ++ | + | + | + | + | ++ |
| | K1093G | L318G/M320R/S332P/F387I/I390A/ | | | | | | |
| | | V393G/V534I/S543P/M558W/ | | | | | | |
| | | M681G/K732W/L749F/L750D/ | | | | | | |
| | | I775G/E809G/K1093G/T1148N/ | | | | | | |
| | | L1248D/L1377D/V1439E | | | | | | |
| 2267/2268 | S543P/E739G/ | V63Q/L171P/H212A/S260T/E306G/ | ++ | + | + | + | ++ | + |
| | K1093A | L318G/M320R/S332P/F387I/I390A/ | | | | | | |
| | | V393G/V534I/S543P/M558W/ | | | | | | |
| | | M681G/K732W/Y738E/E739G/ | | | | | | |
| | | L749F/L750D/I775G/K1093A/ | | | | | | |
| | | T1148N/L1248D/L1377D/V1439E | | | | | | |
| 2269/2270 | S543P/E800V/ | V63Q/L171P/H212A/S260T/E306G/ | ++ | ++ | + | + | + | |
| | K1093G/S1116I | L318G/M320R/S332P/F387I/I390A/ | | | | | | |
| | | V393G/V534I/S543P/M558W/ | | | | | | |
| | | M681G/K732W/Y738E/L749F/ | | | | | | |
| | | L750D/I775G/E800V/K1093G/S1116I/ | | | | | | |
| | | T1148N/L1248D/L1377D/ | | | | | | |
| | | V1439E | | | | | | |
| 2271/2272 | S543P/E809G/ | V63Q/L171P/H212A/S260T/E306G/ | + | + | + | ++ | ++ | ++ |
| | K1093G | L318G/M320R/S332P/F387I/I390A/ | | | | | | |
| | | V393G/V534I/S543P/M558W/ | | | | | | |
| | | M681G/K732W/Y738E/L749F/ | | | | | | |
| | | L750D/I775G/E809G/K1093G/T1148N/ | | | | | | |
| | | L1248D/L1377D/V1439E | | | | | | |
| 2273/2274 | S543P/E809G/ | V63Q/L171P/H212A/S260T/E306G/ | + | ++ | + | ++ | + | |
| | K1093G/S1116I | L318G/M320R/S332P/F387I/I390A/ | | | | | | |
| | | V393G/V534I/S543P/M558W/ | | | | | | |
| | | M681G/K732W/Y738E/L749F/ | | | | | | |
| | | L750D/I775G/E809G/K1093G/S1116I/ | | | | | | |
| | | T1148N/L1248D/L1377D/ | | | | | | |
| | | V1439E | | | | | | |
| 2275/2276 | S543P/G681N/E738Y/ | V63Q/L171P/H212A/S260T/E306G/ | + | ++ | + | ++ | + | ++ |
| | E739G/K752E/ | L318G/M320R/S332P/F387I/I390A/ | | | | | | |
| | E809G/K1093G | V393G/V534I/S543P/M558W/ | | | | | | |
| | | M681N/K732W/E739G/L749F/ | | | | | | |
| | | L750D/K752E/I775G/E809G/K1093G/ | | | | | | |
| | | T1148N/L1248D/L1377D/ | | | | | | |
| | | V1439E | | | | | | |
| 2277/2278 | S543P/G681N/E738Y/ | V63Q/L171P/H212A/S260T/E306G/ | ++ | ++ | + | ++ | ++ | |
| | E739G/K752E/ | L318G/M320R/S332P/F387I/I390A/ | | | | | | |
| | K1093G | V393G/V534I/S543P/M558W/ | | | | | | |
| | | M681N/K732W/E739G/L749F/ | | | | | | |
| | | L750D/K752E/I775G/K1093G/T1148N/ | | | | | | |
| | | L1248D/L1377D/V1439E | | | | | | |
| 2279/2280 | S543P/G681N/E739G/ | V63Q/L171P/H212A/S260T/E306G/ | +++ | ++ | + | ++ | ++ | |
| | K752E/E800V/ | L318G/M320R/S332P/F387I/I390A/ | | | | | | |
| | Y805Q/E809G/ | V393G/V534I/S543P/M558W/ | | | | | | |
| | K1093G | M681N/K732W/Y738E/E739G/ | | | | | | |
| | | L749F/L750D/K752E/I775G/E800V/ | | | | | | |
| | | Y805Q/E809G/K1093G/T1148N/ | | | | | | |
| | | L1248D/L1377D/V1439E | | | | | | |
| 2281/2282 | S543P/G681N/S700E/ | V63Q/L171P/H212A/S260T/E306G/ | + | ++ | + | + | + | + |
| | E738Y/E739G/ | L318G/M320R/S332P/F387I/I390A/ | | | | | | |
| | E800V/K1093G/ | V393G/V534I/S543P/M558W/ | | | | | | |
| | S1116I | M681N/S700E/K732W/E739G/ | | | | | | |
| | | L749F/L750D/I775G/E800V/K1093G/ | | | | | | |
| | | S1116I/T1148N/L1248D/L1377D/ | | | | | | |
| | | V1439E | | | | | | |
| 2283/2284 | S543P/G681N/S700E/ | V63Q/L171P/H212A/S260T/E306G/ | +++ | ++ | + | + | + | + |
| | E738Y/E739G/ | L318G/M320R/S332P/F387I/I390A/ | | | | | | |
| | Y805Q/D808S/ | V393G/V534I/S543P/M558W/ | | | | | | |
| | E809G | M681N/S700E/K732W/E739G/ | | | | | | |
| | | L749F/L750D/I775G/Y805Q/D808S/ | | | | | | |
| | | E809G/T1148N/L1248D/L1377D/ | | | | | | |
| | | V1439E | | | | | | |
| 2285/2286 | S543P/G681N/S700E/ | V63Q/L171P/H212A/S260T/E306G/ | + | ++ | + | + | + | ++ |
| | K752E | L318G/M320R/S332P/F387I/I390A/ | | | | | | |
| | | V393G/V534I/S543P/M558W/ | | | | | | |
| | | M681N/S700E/K732W/Y738E/ | | | | | | |
| | | L749F/L750D/K752E/I775G/T1148N/ | | | | | | |
| | | L1248D/L1377D/V1439E | | | | | | |
| 2287/2288 | S543P/G681N/S700E/ | V63Q/L171P/H212A/S260T/E306G/ | + | + | + | + | + | ++ |
| | K752E/E809G/ | L318G/M320R/S332P/F387I/I390A/ | | | | | | |
| | K1093G | V393G/V534I/S543P/M558W/ | | | | | | |
| | | M681N/S700E/K732W/Y738E/ | | | | | | |
| | | L749F/L750D/K752E/I775G/E809G/ | | | | | | |
| | | K1093G/T1148N/L1248D/L1377D/ | | | | | | |
| | | V1439E | | | | | | |
| 2289/2290 | S543P/K1093G | V63Q/L171P/H212A/S260T/E306G/ | ++ | + | + | ++ | ++ | ++ |
| | | L318G/M320R/S332P/F387I/I390A/ | | | | | | |
| | | V393G/V534I/S543P/M558W/ | | | | | | |
| | | M681G/K732W/Y738E/L749F/ | | | | | | |
| | | L750D/I775G/K1093G/T1148N/ | | | | | | |
| | | L1248D/L1377D/V1439E | | | | | | |
| 2291/2292 | S543P/S700E | V63Q/L171P/H212A/S260T/E306G/ | +++ | ++ | + | ++ | + | |
| | | L318G/M320R/S332P/F387I/I390A/ | | | | | | |
| | | V393G/V534I/S543P/M558W/ | | | | | | |
| | | M681G/S700E/K732W/Y738E/ | | | | | | |
| | | L749F/L750D/I775G/T1148N/L1248D/ | | | | | | |
| | | L1377D/V1439E | | | | | | |
| 2293/2294 | S543P/S700E/E738Y | V63Q/L171P/H212A/S260T/E306G/ | ++ | ++ | + | + | + | ++ |
| | E739G/K752E/ | L318G/M320R/S332P/F387I/I390A/ | | | | | | |
| | E800V/K1093G/ | V393G/V534I/S543P/M558W/ | | | | | | |
| | S1116I | M681G/S700E/K732W/E739G/ | | | | | | |
| | | L749F/L750D/K752E/I775G/E800V/ | | | | | | |
| | | K1093G/S1116I/T1148N/L1248D/ | | | | | | |
| | | L1377D/V1439E | | | | | | |
| 2295/2296 | S543P/S700E/ | V63Q/L171P/H212A/S260T/E306G/ | + | ++ | + | ++ | ++ | |
| | E809G | L318G/M320R/S332P/F387I/I390A/ | | | | | | |
| | | V393G/V534I/S543P/M558W/ | | | | | | |
| | | M681G/S700E/K732W/Y738E/ | | | | | | |
| | | L749F/L750D/I775G/E809G/T1148N/ | | | | | | |
| | | L1248D/L1377D/V1439E | | | | | | |
| 2297/2298 | S543P/S700E/ | V63Q/L171P/H212A/S260T/E306G/ | + | ++ | + | ++ | + | ++ |
| | K1093G | L318G/M320R/S332P/F387I/I390A/ | | | | | | |
| | | V393G/V534I/S543P/M558W/ | | | | | | |
| | | M681G/S700E/K732W/Y738E/ | | | | | | |
| | | L749F/L750D/I775G/K1093G/T1148N/ | | | | | | |
| | | L1248D/L1377D/V1439E | | | | | | |
| 2299/2300 | S543P/S700E/ | V63Q/L171P/H212A/S260T/E306G/ | + | ++ | + | + | + | ++ |
| | K752E | L318G/M320R/S332P/F387I/I390A/ | | | | | | |
| | | V393G/V534I/S543P/M558W/ | | | | | | |
| | | M681G/S700E/K732W/Y738E/ | | | | | | |
| | | L749F/L750D/K752E/I775G/T1148N/ | | | | | | |
| | | L1248D/L1377D/V1439E | | | | | | |
| 2301/2302 | S543P/S700E/K752E/ | V63Q/L171P/H212A/S260T/E306G/ | + | ++ | + | + | + | ++ |
| | E800V/K1093G | L318G/M320R/S332P/F387I/I390A/ | | | | | | |
| | | V393G/V534I/S543P/M558W/ | | | | | | |
| | | M681G/S700E/K732W/Y738E/ | | | | | | |
| | | L749F/L750D/K752E/I775G/E800V/ | | | | | | |
| | | K1093G/T1148N/L1248D/L1377D/ | | | | | | |
| | | V1439E | | | | | | |
| 2303/2304 | S543P/S700E/K752E/ | V63Q/L171P/H212A/S260T/E306G/ | ++ | ++ | + | + | + | ++ |
| | E809G/S1116I | L318G/M320R/S332P/F387I/I390A/ | | | | | | |
| | | V393G/V534I/S543P/M558W/ | | | | | | |
| | | M681G/S700E/K732W/Y738E/ | | | | | | |
| | | L749F/L750D/K752E/I775G/E809G/ | | | | | | |
| | | S1116I/T1148N/L1248D/L1377D/ | | | | | | |
| | | V1439E | | | | | | |
| 2305/2306 | S543P/S700E/ | V63Q/L171P/H212A/S260T/E306G/ | + | ++ | + | ++ | + | +++ |
| | K752E/K1093G | L318G/M320R/S332P/F387I/I390A/ | | | | | | |
| | | V393G/V534I/S543P/M558W/ | | | | | | |
| | | M681G/S700E/K732W/Y738E/ | | | | | | |
| | | L749F/L750D/K752E/I775G/K1093G/ | | | | | | |
| | | T1148N/L1248D/L1377D/ | | | | | | |
| | | V1439E | | | | | | |
| 2307/2308 | S700E | V63Q/L171P/H212A/S260T/E306G/ | +++ | + | + | + | + | |
| | | L318G/M320R/S332P/F387I/I390A/ | | | | | | |
| | | V393G/V534I/M558W/M681G/ | | | | | | |
| | | S700E/K732W/Y738E/L749F/ | | | | | | |
| | | L750D/I775G/T1148N/L1248D/ | | | | | | |
| | | L1377D/V1439E | | | | | | |
| 2309/2310 | S700E/E738Y/E739G/ | V63Q/L171P/H212A/S260T/E306G/ | +++ | ++ | + | ++ | ++ | |
| | E800V/K1093G | L318G/M320R/S332P/F387I/I390A/ | | | | | | |
| | | V393G/V534I/M558W/M681G/ | | | | | | |
| | | S700E/K732W/E739G/L749F/ | | | | | | |
| | | L750D/I775G/E800V/K1093G/ | | | | | | |
| | | T1148N/L1248D/L1377D/V1439E | | | | | | |
| 2311/2312 | S700E/E738Y/E739G/ | V63Q/L171P/H212A/S260T/E306G/ | +++ | + | + | + | + | |
| | K799S/K1093G | L318G/M320R/S332P/F387I/I390A/ | | | | | | |
| | | V393G/V534I/M558W/M681G/ | | | | | | |
| | | S700E/K732W/E739G/L749F/ | | | | | | |
| | | L750D/I775G/K799S/K1093G/ | | | | | | |
| | | T1148N/L1248D/L1377D/V1439E | | | | | | |
| 2313/2314 | S700E/E738Y/E800V/ | V63Q/L171P/H212A/S260T/E306G/ | +++ | ++ | + | ++ | ++ | +++ |
| | K1093G | L318G/M320R/S332P/F387I/I390A/ | | | | | | |
| | | V393G/V534I/M558W/M681G/ | | | | | | |
| | | S700E/K732W/L749F/L750D/ | | | | | | |
| | | I775G/E800V/K1093G/T1148N/ | | | | | | |
| | | L1248D/L1377D/V1439E | | | | | | |
| 2315/2316 | S700E/E738Y/ | V63Q/L171P/H212A/S260T/E306G/ | + | + | + | + | + | ++ |
| | S1116I | L318G/M320R/S332P/F387I/I390A/ | | | | | | |
| | | V393G/V534I/M558W/M681G/ | | | | | | |
| | | S700E/K732W/L749F/L750D/ | | | | | | |
| | | I775G/S1116I/T1148N/L1248D/ | | | | | | |
| | | L1377D/V1439E | | | | | | |
| 2317/2318 | S700E/E809G | V63Q/L171P/H212A/S260T/E306G/ | ++ | ++ | + | ++ | ++ | ++ |
| | | L318G/M320R/S332P/F387I/I390A/ | | | | | | |
| | | V393G/V534I/M558W/M681G/ | | | | | | |
| | | S700E/K732W/Y738E/L749F/ | | | | | | |
| | | L750D/I775G/E809G/T1148N/ | | | | | | |
| | | L1248D/L1377D/V1439E | | | | | | |
| 2319/2320 | S700E/E809G/ | V63Q/L171P/H212A/S260T/E306G/ | ++ | ++ | + | ++ | ++ | ++ |
| | K1093G | L318G/M320R/S332P/F387I/I390A/ | | | | | | |
| | | V393G/V534I/M558W/M681G/ | | | | | | |
| | | S700E/K732W/Y738E/L749F/ | | | | | | |
| | | L750D/I775G/E809G/K1093G/ | | | | | | |
| | | T1148N/L1248D/L1377D/V1439E | | | | | | |
| 2321/2322 | S700E/K1093G | V63Q/L171P/H212A/S260T/E306G/ | ++ | ++ | + | ++ | ++ | ++ |
| | | L318G/M320R/S332P/F387I/I390A/ | | | | | | |
| | | V393G/V534I/M558W/M681G/ | | | | | | |
| | | S700E/K732W/Y738E/L749F/ | | | | | | |
| | | L750D/I775G/K1093G/T1148N/ | | | | | | |
| | | L1248D/L1377D/V1439E | | | | | | |
| 2323/2324 | S700E/K1093G/ | V63Q/L171P/H212A/S260T/E306G/ | ++ | + | + | ++ | ++ | ++ |
| | S1116I | L318G/M320R/S332P/F387I/I390A/ | | | | | | |
| | | V393G/V534I/M558W/M681G/ | | | | | | |
| | | S700E/K732W/Y738E/L749F/ | | | | | | |
| | | L750D/I775G/K1093G/S1116I/ | | | | | | |
| | | T1148N/L1248D/L1377D/V1439E | | | | | | |
| 2325/2326 | S700E/K752E | V63Q/L171P/H212A/S260T/E306G/ | ++ | ++ | + | + | + | |
| | | L318G/M320R/S332P/F387I/I390A/ | | | | | | |
| | | V393G/V534I/M558W/M681G/ | | | | | | |
| | | S700E/K732W/Y738E/L749F/ | | | | | | |
| | | L750D/K752E/I775G/T1148N/ | | | | | | |
| | | L1248D/L1377D/V1439E | | | | | | |
| 2327/2328 | S700E/K752E/D808S/ | V63Q/L171P/H212A/S260T/E306G/ | ++ | ++ | + | ++ | + | ++ |
| | E809G/K1093G/ | L318G/M320R/S332P/F387I/I390A/ | | | | | | |
| | S1116I | V393G/V534I/M558W/M681G/ | | | | | | |
| | | S700E/K732W/Y738E/L749F/ | | | | | | |
| | | L750D/K752E/I775G/D808S/E809G/ | | | | | | |
| | | K1093G/S1116I/T1148N/L1248D/ | | | | | | |
| | | L1377D/V1439E | | | | | | |
| 2329/2330 | S700E/K752E/ | V63Q/L171P/H212A/S260T/E306G/ | ++ | ++ | ++ | + | + | |
| | E809G/K1093G | L318G/M320R/S332P/F387I/I390A/ | | | | | | |
| | | V393G/V534I/M558W/M681G/ | | | | | | |
| | | S700E/K732W/Y738E/L749F/ | | | | | | |
| | | L750D/K752E/I775G/E809G/K1093G/ | | | | | | |
| | | T1148N/L1248D/L1377D/ | | | | | | |
| | | V1439E | | | | | | |
| 2331/2332 | S700E/K752E/ | V63Q/L171P/H212A/S260T/E306G/ | + | ++ | + | + | + | ++ |
| | K799S/S1116I | L318G/M320R/S332P/F387I/I390A/ | | | | | | |
| | | V393G/V534I/M558W/M681G/ | | | | | | |
| | | S700E/K732W/Y738E/L749F/ | | | | | | |
| | | L750D/K752E/I775G/K799S/S1116I/ | | | | | | |
| | | T1148N/L1248D/L1377D/ | | | | | | |
| | | V1439E | | | | | | |
| 2333/2334 | S700E/K752E/ | V63Q/L171P/H212A/S260T/E306G/ | + | ++ | ++ | + | + | ++ |
| | Y805Q/E809G/ | L318G/M320R/S332P/F387I/I390A/ | | | | | | |
| | K1093A | V393G/V534I/M558W/M681G/ | | | | | | |
| | | S700E/K732W/Y738E/L749F/ | | | | | | |
| | | L750D/K752E/I775G/Y805Q/E809G/ | | | | | | |
| | | K1093A/T1148N/L1248D/ | | | | | | |
| | | L1377D/V1439E | | | | | | |
| 2335/2336 | S700E/K799S/ | V63Q/L171P/H212A/S260T/E306G/ | + | ++ | + | ++ | + | ++ |
| | E800V | L318G/M320R/S332P/F387I/I390A/ | | | | | | |
| | | V393G/V534I/M558W/M681G/ | | | | | | |
| | | S700E/K732W/Y738E/L749F/ | | | | | | |
| | | L750D/I775G/K799S/E800V/T1148N/ | | | | | | |
| | | L1248D/L1377D/V1439E | | | | | | |
| 2337/2338 | S700E/S1116I | V63Q/L171P/H212A/S260T/E306G/ | ++ | + | + | + | + | |
| | | L318G/M320R/S332P/F387I/I390A/ | | | | | | |
| | | V393G/V534I/M558W/M681G/ | | | | | | |
| | | S700E/K732W/Y738E/L749F/ | | | | | | |
| | | L750D/I775G/S1116I/T1148N/ | | | | | | |
| | | L1248D/L1377D/V1439E | | | | | | |
| 2339/2340 | S700E/Y805Q/D808S/ | V63Q/L171P/H212A/S260T/E306G/ | ++ | ++ | ++ | ++ | + | ++ |
| | E809G/E1076S | L318G/M320R/S332P/F387I/I390A/ | | | | | | |
| | | V393G/V534I/M558W/M681G/ | | | | | | |
| | | S700E/K732W/Y738E/L749F/ | | | | | | |
| | | L750D/I775G/Y805Q/D808S/E809G/ | | | | | | |
| | | E1076S/T1148N/L1248D/L1377D/ | | | | | | |
| | | V1439E | | | | | | |
| 2341/2342 | V514L/S543P/G681N/ | V63Q/L171P/H212A/S260T/E306G/ | + | + | + | ++ | + | ++ |
| | Y805Q/D808S/ | L318G/M320R/S332P/F387I/I390A/ | | | | | | |
| | E809G | V393G/V514L/V534I/S543P/ | | | | | | |
| | | M558W/M681N/K732W/Y738E/ | | | | | | |
| | | L749F/L750D/I775G/Y805Q/D808S/ | | | | | | |
| | | E809G/T1148N/L1248D/L1377D/ | | | | | | |
| | | V1439E | | | | | | |
| 2343/2344 | Y805Q/D808S/ | V63Q/L171P/H212A/S260T/E306G/ | ++ | + | + | + | + | + |
| | E809G | L318G/M320R/S332P/F387I/I390A/ | | | | | | |
| | | V393G/V534I/M558W/M681G/ | | | | | | |
| | | K732W/Y738E/L749F/L750D/ | | | | | | |
| | | I775G/Y805Q/D808S/E809G/T1148N/ | | | | | | |
| | | L1248D/L1377D/V1439E | | | | | | |
| 2345/2346 | Y805Q/E809G/ | V63Q/L171P/H212A/S260T/E306G/ | + | ++ | + | ++ | + | |
| | S1116I | L318G/M320R/S332P/F387I/I390A/ | | | | | | |
| | | V393G/V534I/M558W/M681G/ | | | | | | |
| | | K732W/Y738E/L749F/L750D/ | | | | | | |
| | | I775G/Y805Q/E809G/S1116I/ | | | | | | |
| | | T1148N/L1248D/L1377D/V1439E | | | | | | |

All activities were determined relative to the reference polypeptide of SEQ ID NO: 2072. Levels of increased activity are defined as follows:
“+” 0.9 to 1.1,
“++” >1.1,
“+++” >2,
“++++” >2.5

### Example 12

### Identification of Active FVIII Variants with Reduced Immunogenicity

Putative T-cell epitopes in a B-domain deleted version of wild type human FVIII were identified using the Immune Epitope Database (IEDB; Immune Epitope Database and Analysis Resource website) tools, as known in the art and proprietary statistical analysis tools (See e.g., iedb.org and Vita et al., Nucl. Acids Res., 2010, 38 (Database issue): D854-62, Epub 2009 Nov. 11). The B-domain deleted version of wild type human FVIII (SEQ ID NO: 2) was parsed into all possible 15-mer analysis frames, with each frame overlapping the last by 14 amino acids. The 15-mer analysis frames were evaluated for immunogenic potential by scoring their predicted binding to eight common class II HLA-DR alleles (DRB1*0101, DRB1*0301, DRB1*0401, DRB1*0701, DRB1*0801, DRB1*1101, DRB1*1301, and DRB1*1501), using methods recommended on the IEDB website. Potential T-cell epitope clusters contained within the polypeptide (i.e., sub-regions contained within human FVIII which have an unusually high potential for immunogenicity) were identified using statistical analysis tools, as known in the art. The identified T-cell epitope clusters were screened against the IEDB database of known epitopes.

Active FVIII variants identified in screens from Example 3 for their levels of predicted immunogenicity by evaluating their binding to the eight common Class II HLA-DR alleles. The total immunogenicity score and immunogenic hit count were calculated for each variant. The total immunogenicity score (TIS) reflects the overall predicted immunogenicity of the variant (i.e., a higher score indicates a higher level of predicted immunogenicity). The immunogenic “hit count” (IHC) indicates the number of 15-mer analysis frames with an unusually high potential for immunogenicity (i.e., a higher score indicates a higher potential for immunogenicity). Mutations resulting in a reduced total immunogenicity score and/or an immunogenic hit count compared to the reference sequence were considered to be potential “deimmunizing mutations” and are shown in Tables 12.1, 12.2, 12.3, 12.4, 12.5, 12.6, 12.7, 12.8, and 15.2. A collection of the most deimmunizing mutations were recombined to generate a number of variants that were active and predicted to be significantly less immunogenic than B-domain deleted version of WT human FVIII (SEQ ID NO: 2).

TABLE 12.1
Reduction of Total Immunogenicity Score (TIS) and Immunogenic
Hit Count (IHC) for FVIII Variants Relative to SEQ ID NO: 2

| SEQ ID | | TIS reduction | IHC reduction |
|---|---|---|---|
| NO: | Amino Acid Differences | Relative to | Relative to |
| (nt/aa) | (Relative to SEQ ID NO: 2) | SEQ ID NO: 2 | SEQ ID NO: 2 |
| 1/2 | | | |
| 191/192 | Q37H/Q96R/L171P/Q429L/I708T/K799R/T1148N | ++ | + |
| 193/194 | L171P | + | + |
| 195/196 | L171P/Q429L/K732R/T1148N | + | + |
| 197/198 | Q96R/L171P | ++ | + |
| 199/200 | L171P/E919G/G1151S | + | |
| 201/202 | L171P/T792Y/M797Q/K799R/T1148N | +++ | + |
| 203/204 | L171P/P569T/T1148N | +++ | + |
| 205/206 | Q96R/L171P/Q429L/T792Y/M797T/K799R | | + |
| 207/208 | L171P/Q429L/I708T/L1422I | ++ | + |
| 209/210 | L171P/Q429L | | + |
| 3/4 | T1148N | + | + |
| 211/212 | D321Y/A362P/K732E/M797T/K799R | + | |
| 213/214 | L171P/L1100I | ++ | + |
| 215/216 | I708T/T1148N/A1186E | +++ | + |
| 217/218 | Q429L/L724I/K732E/T1148N | | + |
| 219/220 | L171P/A362E/V795T/M797Q/K799M | ++ | + |
| 221/222 | L171P/Q429L/K732E | | + |
| 223/224 | Q96R/L171P/S643N/M797T/K799R | ++ | + |
| 225/226 | L171P/T792Y/M797T/K799M | + | + |
| 227/228 | R503S | ++ | + |
| 229/230 | L171P/K732E | + | + |
| 231/232 | T1148N/F1193L | + | + |
| 5/6 | P85Q/1708T/T1148N | ++ | + |
| 233/234 | Q96R/L171P/A362E/Q429L/K732R/V795T/M797Q | + | + |
| 7/8 | K798T | + | |
| 9/10 | L706G | ++ | ++ |
| 11/12 | L706A | + | + |
| 13/14 | L171Q | + | |
| 15/16 | Q335E | + | |
| 17/18 | V63R | ++ | |
| 235/236 | K726A | + | |
| 19/20 | K344A | + | |
| 237/238 | F59M | + | |
| 241/242 | A362N | + | |
| 243/244 | H334G | + | |
| 25/26 | F59L | + | |
| 247/248 | V63Q | +++ | ++ |
| 251/252 | L1377D | +++ | + |
| 27/28 | Q429S | + | |
| 31/32 | I1264W | + | |
| 259/260 | V1250T | ++ | + |
| 261/262 | E306G | + | |
| 265/266 | L1377G | ++ | + |
| 269/270 | L1377E | +++ | + |
| 273/274 | L646I | + | |
| 33/34 | M797P | + | |
| 35/36 | L1248S | + | + |
| 37/38 | I1264R | + | |
| 39/40 | Y873T | + | |
| 41/42 | T792Y | + | |
| 43/44 | I708K | + | + |
| 45/46 | K344L | + | |
| 47/48 | F57V | + | |
| 49/50 | Q96L | + | |
| 277/278 | P435Q | + | |
| 51/52 | A362W | ++ | |
| 53/54 | V63G | +++ | ++ |
| 55/56 | Y873L | + | |
| 59/60 | T1148Q | + | |
| 63/64 | M797G | + | |
| 65/66 | L706Y | ++ | ++ |
| 67/68 | L1248R | ++ | + |
| 71/72 | Y65A | + | + |
| 73/74 | F59W | + | |
| 75/76 | Y873Q | + | |
| 77/78 | V795C | + | |
| 279/280 | Q96V | + | |
| 81/82 | F57L | + | |
| 83/84 | Q353S | + | |
| 85/86 | S332Q | + | + |
| 87/88 | I708A | + | |
| 89/90 | G1259T | + | + |
| 91/92 | V63M | + | |
| 93/94 | M356L | ++ | |
| 95/96 | L1248G | + | + |
| 97/98 | L1248P | ++ | + |
| 99/100 | Q436G | + | |
| 101/102 | F57A | + | |
| 103/104 | F59S | + | + |
| 105/106 | T1245R | + | |
| 107/108 | F59R | + | |
| 109/110 | Q96W | + | |
| 111/112 | M797Y | + | |
| 113/114 | K1145R | + | |
| 281/282 | L1377R | + | |
| 115/116 | Q436D | + | |
| 117/118 | F716L | + | |
| 119/120 | S56E | + | |
| 283/284 | L1377S | + | + |
| 285/286 | F59G | + | |
| 287/288 | L171M | + | |
| 121/122 | R773H | + | |
| 289/290 | T792H | + | |
| 129/130 | F59Q | + | + |
| 131/132 | S729T | + | |
| 133/134 | F716K | + | |
| 135/136 | T665G | + | + |
| 137/138 | A362T | ++ | |
| 139/140 | F57T | + | |
| 141/142 | N359G | + | |
| 291/292 | L1248D | + | + |
| 145/146 | V1250A | ++ | + |
| 147/148 | A362V | + | |
| 149/150 | N359E | ++ | |
| 151/152 | F716W | + | |
| 153/154 | S332W | + | ++ |
| 155/156 | I708V | + | |
| 157/158 | V63H | ++ | ++ |
| 159/160 | M797C | + | |
| 295/296 | L706M | + | ++ |
| 161/162 | T61Q | + | + |
| 163/164 | M356N | ++ | |
| 297/298 | L171H | + | |
| 165/166 | V1250S | + | + |
| 167/168 | K798C | + | |
| 169/170 | N433G | + | |
| 299/300 | L1377A | ++ | |
| 301/302 | L1377V | + | |
| 171/172 | V795N | + | |
| 173/174 | Q96I | + | |
| 175/176 | S56P | + | |
| 177/178 | V63N | ++ | ++ |
| 179/180 | T792W | ++ | |
| 181/182 | I1264L | + | |
| 183/184 | V1250P | + | + |
| 185/186 | S332P | + | + |
| 187/188 | F59V | + | |
| 309/310 | F59D | + | + |
| 189/190 | K726L | + | |
| 311/312 | K344E | + | |

TIS reduction was measured as a reduction in the number of counts compared to the reference polypeptide of SEQ ID NO: 2 and are defined as follows: “+” 1-10, “++” >10, “+++” >20.
IHC reduction was measured as a reduction in the number of counts compared to the reference polypeptide of SEQ ID NO: 2 and are defined as follows: “+” 1-3, “++” >3, “+++” >5

TABLE 12.2
Reduction of Total Immunogenicity Score (TIS) and Immunogenic
Hit Count (IHC) for FVIII Variants Relative to SEQ ID NO: 252

| SEQ ID | | TIS reduction | IHC reduction |
|---|---|---|---|
| NO: | Amino Acid Differences | Relative to | Relative to |
| (nt/aa) | (Relative to SEQ ID NO: 252) | SEQ ID NO: 252 | SEQ ID NO: 252 |
| 251/252 | | | |
| 411/412 | I265S | ++ | + |
| 413/414 | L1221T | + | ++ |
| 415/416 | M1380G | + | |
| 417/418 | K752G | ++ | + |
| 419/420 | Y748K | +++ | +++ |
| 423/424 | F1218A | ++ | ++ |
| 425/426 | Y748N | +++ | +++ |
| 427/428 | T1378E | + | |
| 429/430 | L614I | + | + |
| 431/432 | L749F | + | + |
| 433/434 | F387V | ++ | + |
| 435/436 | L750T | + | + |
| 437/438 | F1193G | ++ | + |
| 441/442 | T1378A | + | |
| 443/444 | L750V | | + |
| 445/446 | F387T | +++ | +++ |
| 447/448 | F387Q | +++ | +++ |
| 451/452 | F1037N | ++ | + |
| 453/454 | H1440A | + | |
| 455/456 | M1051S | + | + |
| 457/458 | L1221G | ++ | ++ |
| 459/460 | T1378D | + | |
| 461/462 | H1440S | + | + |
| 463/464 | F387R | ++ | +++ |
| 465/466 | F1193A | + | + |
| 467/468 | F387I | + | + |
| 469/470 | F1218M | ++ | ++ |
| 319/320 | I388A | ++ | ++ |
| 471/472 | I265W | +++ | + |
| 473/474 | H1440E | +++ | +++ |
| 475/476 | V1439L | + | + |
| 477/478 | H1440T | ++ | +++ |
| 479/480 | R612S | | + |
| 481/482 | Y1049W | + | ++ |
| 321/322 | F1043E | +++ | +++ |
| 323/324 | F1043R | ++ | ++ |
| 483/484 | L1221S | + | ++ |
| 325/326 | I610R | ++ | + |
| 327/328 | I1274N | +++ | +++ |
| 329/330 | S1220T | + | + |
| 331/332 | I1442T | + | + |
| 485/486 | S1220D | ++ | ++ |
| 333/334 | F1193L | | + |
| 335/336 | Q621T | + | + |
| 337/338 | L1054C | + | + |
| 339/340 | A755Q | + | + |
| 487/488 | L749G | +++ | +++ |
| 341/342 | Y1041G | ++ | ++ |
| 343/344 | Y748S | +++ | +++ |
| 345/346 | Y1049G | +++ | +++ |
| 347/348 | L1221V | + | ++ |
| 489/490 | L749S | ++ | ++ |
| 491/492 | F1218G | ++ | ++ |
| 497/498 | F1193E | ++ | + |
| 499/500 | I1050S | ++ | ++ |
| 501/502 | S1389W | + | |
| 349/350 | M1051E | +++ | +++ |
| 503/504 | V1439E | +++ | +++ |
| 505/506 | S260T | + | + |
| 507/508 | F1037P | ++ | + |
| 351/352 | R612G | + | + |
| 353/354 | F1037H | ++ | + |
| 355/356 | N616E | ++ | + |
| 509/510 | M1051K | ++ | ++ |
| 511/512 | I1269F | + | + |
| 513/514 | I265R | + | + |
| 357/358 | Y1041H | ++ | +++ |
| 515/516 | M1051R | ++ | ++ |
| 517/518 | F1218L | + | ++ |
| 359/360 | F387S | +++ | +++ |
| 361/362 | S489R | ++ | + |
| 363/364 | Q621M | + | + |
| 365/366 | S489Q | ++ | + |
| 367/368 | L750G | + | + |
| 519/520 | M897R | + | + |
| 521/522 | I388P | +++ | +++ |
| 369/370 | S1224P | + | + |
| 523/524 | Y748G | +++ | +++ |
| 371/372 | A618C | + | + |
| 525/526 | I756P | ++ | +++ |
| 373/374 | N616G | ++ | + |
| 375/376 | R612T | | + |
| 529/530 | I390A | ++ | + |
| 377/378 | I1050Q | ++ | ++ |
| 531/532 | I610L | | + |
| 533/534 | A755G | + | + |
| 535/536 | L749R | + | + |
| 379/380 | T1053A | + | + |
| 381/382 | N616W | + | + |
| 537/538 | Y748V | + | + |
| 383/384 | I896V | + | + |
| 539/540 | M1380A | + | |
| 541/542 | H1440C | ++ | +++ |
| 385/386 | S489F | + | + |
| 543/544 | H397N/F1218M | ++ | ++ |
| 387/388 | I1050P | ++ | ++ |
| 389/390 | M897G | ++ | + |
| 545/546 | F1037M | + | + |
| 547/548 | F1218P | ++ | ++ |
| 549/550 | E757K | + | + |
| 391/392 | L749T | ++ | + |
| 551/552 | V1373S | + | |
| 553/554 | H1440G | ++ | +++ |
| 555/556 | F1193I | + | + |
| 557/558 | V1439T | ++ | +++ |
| 393/394 | I1274V | + | + |
| 395/396 | S1220L | + | + |
| 559/560 | L750D | +++ | +++ |
| 397/398 | S260E | ++ | + |
| 561/562 | I390G | +++ | + |
| 563/564 | I756G | ++ | ++ |
| 399/400 | L481V | + | |
| 565/566 | S1220E | ++ | ++ |
| 401/402 | F613S | + | + |
| 403/404 | F387G | +++ | +++ |
| 405/406 | Q621R | + | + |
| 567/568 | T1378C | + | |
| 407/408 | I756R | ++ | + |
| 569/570 | A618G | ++ | + |
| 573/574 | V63Q/L1248D | +++ | +++ |
| 577/578 | E306G/R355C/L1422I | +++ | |
| 579/580 | L1422I | + | |
| 581/582 | E306G/D346R/I708T | ++ | |
| 583/584 | T1148N | + | + |
| 585/586 | L171P/D346R/L1248D | ++ | + |
| 587/588 | V63Q/M797T/K799R/L1248D/L1422I | +++ | +++ |
| 589/590 | L171P/K799R | + | + |
| 591/592 | A362P | + | |
| 593/594 | L1248D | + | + |
| 595/596 | V63Q | +++ | ++ |
| 597/598 | L171P/E306G/D346R/Q999R/L1248D | ++ | + |
| 599/600 | L171P/T1148N | ++ | + |
| 601/602 | T734S/K799R/L1248D/L1422I | ++ | + |
| 603/604 | V63Q/K732E | +++ | ++ |
| 605/606 | V63Q/L171P/E306G/D346R/K732G | +++ | +++ |
| 607/608 | L171P | + | + |
| 609/610 | L171P/L1422I | ++ | + |
| 611/612 | V63Q/A362P/K732G/K799R/L1248D | +++ | +++ |
| 613/614 | Q999R/T1148N/L1422I | ++ | + |
| 617/618 | E306G/P349G | + | |
| 619/620 | V63Q/L171P/T1148N/G1151S | +++ | +++ |
| 621/622 | E306G | + | |
| 623/624 | K344E/P349G/L354R/V1250T | +++ | + |
| 625/626 | V63G | +++ | ++ |
| 627/628 | V1250T | ++ | + |
| 629/630 | V63R/E306G/K344E/P349G/L354R/R503S/L1248R | ++++ | ++++ |
| 631/632 | F59D/V63S/S332P/P349G/R503S/L706M/I708K | ++++ | ++++ |
| 633/634 | V63S/E306G/K344A/P349G/L354R/L706M/L1248R | ++++ | ++++ |
| 635/636 | L1248R | ++ | + |
| 637/638 | F59D/V63G | +++ | ++ |
| 639/640 | S332P/L706M/I708K/L1248R/V1250T | +++ | ++++ |
| 641/642 | R503S | ++ | + |
| 643/644 | F59D/V63R/P349G/L354R | +++ | ++ |
| 645/646 | F59D/P349G/I708K | ++ | + |
| 647/648 | F59D/E306G/K344E/L1248R | +++ | + |
| 409/410 | V63R | ++ | ++ |
| 649/650 | K344E/L1248R/V1250T | +++ | + |
| 651/652 | I708K | + | + |
| 653/654 | V63R/K344E/P349G | +++ | ++ |
| 655/656 | F59D | + | + |
| 657/658 | V63S/K344A/P349G/L354R | +++ | ++ |
| 659/660 | L706M | + | ++ |
| 661/662 | F59D/E306G/R770W | ++ | + |
| 663/664 | V63G/S347N/L354R/L1248R | ++++ | +++ |
| 665/666 | L354R/R503S | +++ | + |

TIS reduction was measured as a reduction in the number of counts compared to the reference polypeptide of SEQ ID NO: 252 and are defined as follows: “+” 1-10, “++” >10, “+++” >20, “++++” >50, “+++++”.
IHC reduction was measured as a reduction in the number of counts compared to the reference polypeptide of SEQ ID NO: 252 and are defined as follows: “+” 1-3, “++” >3, “+++” >5, “++++” >8

TABLE 12.3
Reduction of Total Immunogenicity Score (TIS) and Immunogenic
Hit Count (IHC) for FVIII Variants Relative to SEQ ID NO: 504

| SEQ ID | | TIS reduction | IHC reduction |
|---|---|---|---|
| NO: | Amino Acid Differences | Relative to | Relative to |
| (nt/aa) | (Relative to SEQ ID NO: 504) | SEQ ID NO: 504 | SEQ ID NO: 504 |
| 503/504 | | | |
| 701/702 | L171P/L1248D/H1440T | ++ | + |
| 703/704 | S260T/T1148N/T1378D | ++ | ++ |
| 705/706 | L171P/S260T/F387I/L750D/L1248D | ++++ | ++++ |
| 707/708 | L171P/S260T | + | ++ |
| 709/710 | L171P/S260T/F387I/L750D/M1051S/T1148N | ++++ | +++++ |
| 711/712 | L171P | + | + |
| 667/668 | L750D | +++ | +++ |
| 713/714 | L750D/M1051S/T1148N | +++ | ++++ |
| 715/716 | L171P/S260T/I756P | +++ | ++++ |
| 669/670 | M1051K | ++ | ++ |
| 717/718 | L171P/L1248D | ++ | + |
| 671/672 | M1051S | + | + |
| 719/720 | L171P/T1148N | ++ | + |
| 721/722 | F387I | + | + |
| 723/724 | F59D | + | + |
| 727/728 | S332P/L750D/L1248D | +++ | ++++ |
| 729/730 | I756P/L1248D | +++ | +++ |
| 673/674 | S260T | + | + |
| 731/732 | L1248D | + | + |
| 675/676 | I756P | ++ | +++ |
| 733/734 | L171P/F387I/L1248D/H1440S | +++ | + |
| 677/678 | F1193A | + | + |
| 735/736 | L750D/H1440S | +++ | +++ |
| 737/738 | F59D/L171P/L750D | +++ | ++++ |
| 739/740 | L171P/F1193A/L1248D | ++ | ++ |
| 679/680 | S332P | + | + |
| 741/742 | L750D/T1148N/F1193A/H1440S | +++ | ++++ |
| 743/744 | T1148N | + | + |
| 745/746 | L171P/F387I/M1051K | +++ | +++ |
| 747/748 | Y748G | +++ | +++ |
| 681/682 | M897R | + | + |
| 683/684 | F387Q | +++ | +++ |
| 749/750 | E306G | + | |
| 751/752 | E306G/F387R/I390A/L749F/S1220D | ++++ | ++++ |
| 685/686 | F1037N | ++ | + |
| 753/754 | I390A | ++ | + |
| 755/756 | E306G/I390A/F1037N | +++ | + |
| 757/758 | E306G/R612S/L749F | + | + |
| 759/760 | F387V/1390A/L749F | +++ | +++ |
| 761/762 | V63S/I390A/R503S/L749F | ++++ | ++++ |
| 763/764 | F387R/I390A/L749R | +++ | +++ |
| 765/766 | S1220D | ++ | ++ |
| 687/688 | F387R | ++ | +++ |
| 767/768 | V63S/E306G | +++ | ++ |
| 689/690 | E306G/L749F | + | + |
| 769/770 | L749F | + | + |
| 771/772 | E306G/I390A/L749F/F1037N | +++ | + |
| 773/774 | F387V | ++ | + |
| 775/776 | E306G/L749F/S1220D | +++ | ++ |
| 777/778 | E306G/F387V/I390A/R612S/L749F/F1037N | ++++ | ++++ |
| 691/692 | I1270V | + | + |
| 693/694 | S489E | +++ | + |
| 779/780 | F328S | ++ | +++ |
| 695/696 | Q487N | ++ | + |
| 697/698 | A488G | ++ | + |
| 699/700 | Q903E | ++ | +++ |

TIS reduction was measured as a reduction in the number of counts compared to the reference polypeptide of SEQ ID NO: 504 and are defined as follows: “+” 1-10, “++” >10, “+++” >20, “++++” >50, “+++++” >75.
IHC reduction was measured as a reduction in the number of counts compared to the reference polypeptide of SEQ ID NO: 504 and are defined as follows: “+” 1-3, “++” >3, “+++” >5, “++++” >8, “+++++” >15

TABLE 12.4
Reduction of Total Immunogenicity Score (TIS) and Immunogenic
Hit Count (IHC) for FVIII Variants Relative to SEQ ID NO: 706

| SEQ ID | | TIS reduction | IHC reduction |
|---|---|---|---|
| NO: | Amino Acid Differences | Relative to | Relative to |
| (nt/aa) | (Relative to SEQ ID NO: 706) | SEQ ID NO: 706 | SEQ ID NO: 706 |
| 705/706 | | | |
| 819/820 | S332P/I387R/I390A/T1148N | +++ | +++ |
| 821/822 | F328S/I387R/L749F | +++ | ++++ |
| 781/782 | M1051K | ++ | ++ |
| 823/824 | L749F | + | |
| 783/784 | L1221G | ++ | ++ |
| 825/826 | E306G/F328S/S332P/I390A/M1051R/F1193A/F1218G | ++++ | ++++ |
| 827/828 | V63Q/E306G/S332P/M1051R/T1148N | ++++ | ++++ |
| 829/830 | V63Q/E306G/S332P/I387T/M1051K | ++++ | +++++ |
| 831/832 | I387T/I390A/M1051R | +++ | ++++ |
| 833/834 | D186A/E306G/F328S/S332P/I390A/L749F/M1051K/T1148N/ | ++++ | ++++ |
| | F1193A | | |
| 835/836 | I387T/I390A/I756P/M1051R | ++++ | ++++ |
| 837/838 | E306G/I390A/M1051R | +++ | +++ |
| 839/840 | E306G/S332P/I390A/L749F | +++ | ++ |
| 785/786 | F1218G | ++ | ++ |
| 841/842 | E306G/I387R/I390A/T1148N | +++ | +++ |
| 843/844 | E306G/F328S/S332P/I387T/I390A/L749F/M1051R/T1148N/ | +++++ | ++++ |
| | F1193A | | |
| 845/846 | S332P/I387R/I390A/L749F/T1148N | +++ | +++ |
| 847/848 | I387R/I390A/L749F | +++ | ++ |
| 787/788 | I387R | ++ | ++ |
| 849/850 | E306G/I387T/L749F/M1051K/T1148N | +++ | ++++ |
| 789/790 | M1051R | ++ | ++ |
| 851/852 | I387R/L749F | ++ | ++ |
| 853/854 | E306G/Q945K/M1051R/F1193A/L1221G | +++ | ++ |
| 855/856 | E306G/I390A/L749F/M1051K | +++ | +++ |
| 857/858 | V63Q/E306G/S332P/I390A | +++ | ++++ |
| 791/792 | L749F/F1218G | ++ | ++ |
| 859/860 | E306G/I387T/I390A/L749F/T1148N/F1193A/L1221G | ++++ | ++++ |
| 861/862 | E306G/S332P/I387R/I390A/L749F/F1218M | ++++ | ++++ |
| 793/794 | I387T | ++ | ++ |
| 863/864 | V63Q/E306G/S332P/I390A/M1051R/T1148N/F1193A/ | +++++ | +++++ |
| | F1218G | | |
| 795/796 | S332P | + | + |
| 867/868 | A755S | + | |
| 875/876 | M797Y | + | |
| 797/798 | M897K | + | + |
| 877/878 | N754W | + | |
| 879/880 | I793G | + | |
| 881/882 | N753D | + | |
| 883/884 | R922W | + | |
| 885/886 | I896L | + | |
| 799/800 | T696E | + | + |
| 889/890 | I775G | + | |
| 801/802 | F816W | + | + |
| 803/804 | F824W | ++ | +++ |
| 805/806 | A826G | ++ | +++ |
| 891/892 | S751E | + | |
| 807/808 | V393W | ++ | + |
| 809/810 | I825L | + | + |
| 811/812 | M897R | + | + |
| 895/896 | K752G | + | |
| 899/900 | K938V | + | |
| 903/904 | A755G | + | |
| 905/906 | V393G/A407V | +++ | ++ |
| 813/814 | F816V | + | + |
| 907/908 | M797W | + | |
| 909/910 | K819V | + | + |
| 911/912 | V393G | +++ | ++ |
| 913/914 | I787W | + | |
| 921/922 | G924P | + | |
| 815/816 | F698W | + | ++ |
| 931/932 | S392A | + | + |
| 933/934 | H397G | + | |
| 817/818 | V393Q | + | + |

TIS reduction was measured as a reduction in the number of counts compared to the reference polypeptide of SEQ ID NO: 706 and are defined as follows: “+” 1-10, “++” >10, “+++” >20, “++++” >50, “+++++” >75.
IHC reduction was measured as a reduction in the number of counts compared to the reference polypeptide of SEQ ID NO: 706 and are defined as follows: “+” 1-3, “++” >3, “+++” >5, “++++” >8, “+++++” >15

TABLE 12.5
Reduction of Total Immunogenicity Score (TIS) and Immunogenic
Hit Count (IHC) for FVIII Variants Relative to SEQ ID NO: 840

| | Amino Acid | | |
|---|---|---|---|
| | Differences | TIS reduction | IHC reduction |
| SEQ ID | (Relative to | Relative to | Relative to |
| NO: (nt/aa) | SEQ ID NO: 840) | SEQ ID NO: 840 | SEQ ID NO: 840 |
| 839/840 | | | |
| 1029/1030 | L622D | + | + |
| 1031/1032 | P617E | + | + |
| 1033/1034 | P617N | + | + |
| 935/936 | V620N | + | + |
| 1035/1036 | P907D | + | + |
| 1037/1038 | N1040E | + | + |
| 1039/1040 | P617D | + | + |
| 1041/1042 | Q611E | + | + |
| 937/938 | P491E | + | + |
| 1043/1044 | V620P | + | + |
| 1045/1046 | P617G | + | + |
| 1047/1048 | A488G | ++ | + |
| 1049/1050 | P1192D | + | + |
| 1051/1052 | S1194P | + | + |
| 1053/1054 | G619E | + | + |
| 939/940 | M1229D | +++ | ++ |
| 1055/1056 | P615D | ++ | + |
| 941/942 | P615N | + | + |
| 1057/1058 | F16A | + | + |
| 943/944 | I1283S | +++ | ++ |
| 945/946 | H822S | + | |
| 1063/1064 | L211K | ++ | + |
| 1065/1066 | M836W | + | + |
| 947/948 | F328G | +++ | ++ |
| 1067/1068 | F555R | + | + |
| 951/952 | K818W | + | + |
| 1069/1070 | V536A | + | + |
| 955/956 | F555G/E684G | ++ | + |
| 1071/1072 | I527A | + | + |
| 1073/1074 | F555E | ++ | + |
| 957/958 | V828K | | + |
| 1075/1076 | H841W | | + |
| 959/960 | R550G | + | + |
| 1077/1078 | M320Q | + | + |
| 1079/1080 | S837G | ++ | + |
| 1081/1082 | M558R | ++ | ++ |
| 1083/1084 | F555G | ++ | ++ |
| 1085/1086 | L211R | ++ | + |
| 961/962 | R830M | | + |
| 963/964 | S839A | + | + |
| 965/966 | H822T | + | + |
| 1087/1088 | V64G | + | ++ |
| 1089/1090 | M320R | | + |
| 1091/1092 | T533Y | ++ | ++ |
| 1095/1096 | F16V | + | |
| 1097/1098 | I1283E | +++ | +++ |
| 1099/1100 | V536M | + | + |
| 969/970 | L14G | ++ | ++ |
| 1101/1102 | F312L | | + |
| 1103/1104 | M681N | +++ | ++ |
| 1107/1108 | V536R | + | + |
| 1109/1110 | R15G | + | + |
| 1111/1112 | V536P | ++ | ++ |
| 973/974 | M836V | + | + |
| 1113/1114 | M558W | ++ | ++ |
| 1115/1116 | M558E | +++ | ++ |
| 975/976 | S839P | + | + |
| 977/978 | K1210W | +++ | ++ |
| 979/980 | Y1281R | ++ | ++ |
| 981/982 | V64D | +++ | ++ |
| 985/986 | V682R | + | + |
| 987/988 | L14M | + | |
| 989/990 | L318C | +++ | +++ |
| 1119/1120 | S839G | ++ | + |
| 1123/1124 | L13G | ++ | + |
| 1125/1126 | K1210M | + | + |
| 1127/1128 | H1156G | + | + |
| 1129/1130 | M558L | + | + |
| 1131/1132 | S839K | ++ | + |
| 1133/1134 | L211P | ++ | + |
| 1135/1136 | L211G | ++ | + |
| 1137/1138 | L211Q | ++ | + |
| 1139/1140 | F16D | +++ | ++++ |
| 995/996 | K818G | + | + |
| 1141/1142 | M320G | ++ | + |
| 997/998 | M836G | ++ | + |
| 1143/1144 | Y683W | + | + |
| 1145/1146 | Y24V | + | |
| 1147/1148 | V534I | + | + |
| 1149/1150 | M681A | +++ | ++ |
| 1001/1002 | M836R | + | + |
| 1151/1152 | P840G | | + |
| 1153/1154 | F1160R | +++ | + |
| 1155/1156 | M681C | +++ | ++ |
| 1157/1158 | F328Q | ++ | ++ |
| 1159/1160 | R15S | ++ | + |
| 1005/1006 | T533V | + | + |
| 1007/1008 | S19G | ++ | +++ |
| 1161/1162 | V536K | + | + |
| 1163/1164 | V536G | + | ++ |
| 1165/1166 | T533R | + | ++ |
| 1167/1168 | M681E | +++ | ++ |
| 1011/1012 | Q817D | ++ | + |
| 1013/1014 | L831W | + | +++ |
| 1169/1170 | V536W | ++ | ++ |
| 1171/1172 | H1156N | + | + |
| 1173/1174 | F16P | ++ | +++ |
| 1175/1176 | Q817A | + | + |
| 1177/1178 | F18R | + | + |
| 1179/1180 | Y551W | + | |
| 1181/1182 | F555T | ++ | ++ |
| 1017/1018 | H1156R | | + |
| 1183/1184 | M681G | +++ | ++ |
| 1185/1186 | H212A | + | + |
| 1187/1188 | F16G | ++ | ++ |
| 1189/1190 | S838G | + | + |
| 1019/1020 | R830V | | ++ |
| 1191/1192 | M681R | + | + |
| 1021/1022 | V393G/A747D | ++ | + |
| 1193/1194 | V393G | ++ | + |
| 1195/1196 | V393G/A747D/K752G/Q811F/K819V/Q923M/ | +++ | +++ |
| | T1148N | ++ | + |
| 1197/1198 | V393G/S751Y/I775G | | |
| 1023/1024 | N753D | ++ | |
| 1199/1200 | V63Q/V393G/K732W/I775G/T1148N | +++ | ++++ |
| 1201/1202 | V393G/K732W/T1148N | +++ | ++ |
| 1203/1204 | I775G/T1148N | + | + |
| 1205/1206 | V393G/K752L/N753D/I775G/I793G/M797W | +++ | + |
| 1207/1208 | V393G/Q923M | ++ | + |
| 1209/1210 | V393G/Q923M/T1148N | +++ | ++ |
| 1211/1212 | V393G/A747D/S751Y | ++ | + |
| 1025/1026 | V393G/K732W/S751Y | ++ | + |
| 1213/1214 | V393G/A747D/N753D/I775G/T1148N | +++ | ++ |
| 1215/1216 | K752L/Q923M/M1051R | ++ | ++ |
| 1217/1218 | V63Q/I775G | +++ | ++ |
| 1219/1220 | V63Q/V393G/K752L/I775G/I793G/M1051R/T1053R | ++++ | ++++ |
| 1221/1222 | K752G/N753D/I775G/I793G/K819V | +++ | + |
| 1027/1028 | V63Q/M797W | +++ | ++ |
| 1223/1224 | V63Q/M1051R | +++ | ++++ |
| 1225/1226 | V63Q/A747D/K752G/N753D/I775G/Q811F/K819V/ | ++++ | ++++ |
| | Q923M/M1051R | | |
| 1227/1228 | V393G/K752G/I787W | +++ | + |
| 1231/1232 | V63Q/T1148N | +++ | +++ |
| 1233/1234 | V63H/A747D/K752G/N753D/K819V/Q923M/ | +++ | ++++ |
| | T1148N | | |
| 1235/1236 | T1148N | + | + |
| 1237/1238 | A747D/K752L/N753D/I793G/Q811F | ++ | |
| 1239/1240 | V63Q | +++ | ++ |
| 1241/1242 | M797W/T1148N | ++ | + |
| 1243/1244 | S751Y/K752L/N753D/M1051R | +++ | ++ |
| 1245/1246 | V63Q/K732W/M1051R | +++ | ++++ |
| 1247/1248 | K752G/M797W/S815I/Q923M/M1051R/T1148N | +++ | ++ |
| 1249/1250 | A747D/N753D | ++ | |

TIS reduction was measured as a reduction in the number of counts compared to the reference polypeptide of SEQ ID NO: 840 and are defined as follows: “+” 1-10, “++” >10, “+++” >20, “++++” >50.
IHC reduction was measured as a reduction in the number of counts compared to the reference polypeptide of SEQ ID NO: 840 and are defined as follows: “+” 1-3, “++” >3, “+++” >5, “++++” >8

TABLE 12.6
Reduction of Total Immunogenicity Score (TIS) and Immunogenic
Hit Count (IHC) for FVIII Variants Relative to SEQ ID NO: 1200

| SEQ ID | | TIS reduction | IHC reduction |
|---|---|---|---|
| NO: | Amino Acid Differences | Relative to | Relative to |
| (nt/aa) | (Relative to SEQ ID NO: 1200) | SEQ ID NO: 1200 | SEQ ID NO: 1200 |
| 1199/1200 | | | |
| 1409/1410 | V536P | ++ | ++ |
| 1411/1412 | L211R/V536K/L622D/M681A | +++ | ++++ |
| 1413/1414 | V536K/M558L | ++ | ++ |
| 1415/1416 | L211R/V536R/F555G/P617G/W732K | +++ | ++++ |
| 1417/1418 | L211R/M681R/W732K | +++ | ++ |
| 1419/1420 | L211R/V536K/F555E | +++ | +++ |
| 1421/1422 | V536R | + | + |
| 1423/1424 | F555E | ++ | ++ |
| 1425/1426 | L211R/V536P | +++ | ++ |
| 1427/1428 | L211R/H212P | ++ | + |
| 1251/1252 | M681A | +++ | ++ |
| 1429/1430 | L211R/V536K | +++ | ++ |
| 1431/1432 | L211R/V536K/P617G/M681A/W732K | +++ | ++++ |
| 1433/1434 | L211R | ++ | + |
| 1435/1436 | L211R/V536P/M558L/P617G/M681A | ++++ | ++++ |
| 1437/1438 | F555G/W732K | ++ | ++ |
| 1439/1440 | V536P/F555E/M558L/P617G/W732K | +++ | ++++ |
| 1253/1254 | L622D | + | + |
| 1255/1256 | P617G | + | + |
| 1441/1442 | V536K/P617G/M681A/W732K | +++ | +++ |
| 1443/1444 | V536K/F555G | +++ | +++ |
| 1257/1258 | M558L | + | + |
| 1445/1446 | V536P/L622D/M681A | +++ | ++++ |
| 1447/1448 | W732K | + | |
| 1259/1260 | V536K | + | + |
| 1449/1450 | H212A/M320Q/T533R/P617E/M681G | +++ | ++++ |
| 1451/1452 | L211K/F312L/T533R/V534I/P617D/G619E/ | ++++ | +++++ |
| | M681N/S839K | | |
| 1453/1454 | H212A | + | + |
| 1455/1456 | L211K/H212A/M320Q/G619E/M681G/S839K | +++ | ++++ |
| 1457/1458 | M797W/S839K | ++ | + |
| 1459/1460 | H212A/F312L/M320Q/T533R/V534I/P617E/ | +++ | +++++ |
| | M681G | | |
| 1461/1462 | M558W/G619E/M681G | +++ | ++++ |
| 1261/1262 | M681G | +++ | ++ |
| 1463/1464 | M320Q/M558W/M681G | +++ | ++++ |
| 1465/1466 | H212A/M320Q/V534I/G619E/M681G/M797W/ | ++++ | ++++ |
| | S839K | | |
| 1467/1468 | M320R/M558W/G619E/W732K/S839K | +++ | ++++ |
| 1469/1470 | L211K/H212A/F312L/V534I/M558W/G619E/ | ++++ | ++++ |
| | M681N/W732K/S839K | | |
| 1471/1472 | M320R/V534I/M558R/G619E/W732K/M797W | +++ | +++ |
| 1473/1474 | M558W/P617D/G619E/M681N | +++ | ++++ |
| 1475/1476 | L211Q/V534I/M558W/Q611E/M681N | ++++ | ++++ |
| 1477/1478 | L211K/M320R/P617E/G619E/M681G | +++ | ++++ |
| 1479/1480 | M558W/P617D/M681G/M797W | +++ | ++++ |
| 1481/1482 | M320R/V534I/G619E/W732K | ++ | ++ |
| 1483/1484 | L211K/G619E/M681N | +++ | +++ |
| 1263/1264 | L211Q | ++ | + |
| 1485/1486 | L211K/H212A/V534I/M558W/G619E/W732K/ | ++++ | ++++ |
| | S839K | | |
| 1487/1488 | H212A/M320R/G619E/M681N/M797W/S839K | +++ | ++++ |
| 1489/1490 | H212A/M320R/Q611E/S839K | ++ | ++++ |
| 1265/1266 | M320R | | + |
| 1491/1492 | M320Q/M558W/G619E/M681N | +++ | ++++ |
| 1493/1494 | F312L/T533R/P617D/M681N/M797W/S839K | +++ | ++++ |
| 1495/1496 | M558W/Q611E/M681N | +++ | ++++ |
| 1497/1498 | F312L | | + |
| 1499/1500 | L211Q/M320R/M558W/G619E/M681G/W732K/ | ++++ | ++++ |
| | M797W/S839K | | |
| 1501/1502 | M320Q/M558W/W732K/M797W | +++ | ++ |
| 1503/1504 | T533R/M558W/Q611E/G619E/F716L/W732K/ | +++ | ++++ |
| | S839K | | |
| 1505/1506 | F312L/M681G | ++ | ++ |
| 1267/1268 | M558W | ++ | ++ |
| 1507/1508 | M320Q/V534I/G619E/M681N | +++ | +++ |
| 1509/1510 | L211K/M320Q/T533R/V536L/G619E | +++ | +++ |
| 1269/1270 | S839K | ++ | + |
| 1511/1512 | M558W/P617E/M681N/S839K | ++++ | ++++ |
| 1271/1272 | L211K | ++ | + |
| 1513/1514 | H212A/M320R/V534I/M558W/M681G | +++ | ++++ |
| 1515/1516 | T533R/V534I/M681N/S839K | +++ | ++++ |
| 1517/1518 | H212A/V534I/G619E/M681G | +++ | +++ |
| 1519/1520 | P617E/G619E/M681G | +++ | +++ |
| 1521/1522 | L211Q/H212A/M320R/M558W/P617E/M681N | ++++ | ++++ |
| 1523/1524 | H212A/V534I/M558W/M681N/M797W | +++ | ++++ |
| 1525/1526 | H212A/M320R/V534I | + | ++ |
| 1527/1528 | L211K/F312L/G619E | ++ | + |
| 1529/1530 | H212A/F312L/G619E/S839K | ++ | +++ |
| 1531/1532 | T210P/H212A/M320R/M558W/M681G | ++++ | ++++ |
| 1273/1274 | M797W | + | |
| 1275/1276 | T533R | + | ++ |
| 1533/1534 | H212A/F312L/V534I/M681N/M797W/S839K | +++ | ++++ |
| 1535/1536 | L211K/M320R/M558W/S839K | +++ | ++++ |
| 1277/1278 | S751A | + | |
| 1279/1280 | A303T | + | + |
| 1539/1540 | S916L | + | |
| 1549/1550 | T294G | + | + |
| 1285/1286 | K818G | + | + |
| 1551/1552 | K799S | + | |
| 1553/1554 | K799E | + | |
| 1299/1300 | Q921W | + | |
| 1563/1564 | K799Q | + | |
| 1567/1568 | S916M | + | |
| 1305/1306 | L968A | + | |
| 1309/1310 | G924W | + | |
| 1569/1570 | R759S | + | |
| 1313/1314 | F698D | +++ | ++ |
| 1315/1316 | S168G | + | |
| 1319/1320 | K818A | + | + |
| 1321/1322 | K818S | + | + |
| 1323/1324 | R581K | + | |
| 1325/1326 | K818P | + | + |
| 1327/1328 | S916K | + | + |
| 1329/1330 | F698S | +++ | ++ |
| 1331/1332 | A303G | + | + |
| 1333/1334 | S916C | ++ | ++ |
| 1335/1336 | T1428G | + | + |
| 1343/1344 | T311G | + | |
| 1351/1352 | S751D | + | |
| 1355/1356 | K818E | ++ | + |
| 1367/1368 | A1099G | + | |
| 1369/1370 | K799G | + | |
| 1371/1372 | S916I | + | + |
| 1379/1380 | K799P | + | |
| 1383/1384 | S916E | +++ | ++ |
| 1583/1584 | S916T | + | |
| 1385/1386 | T676Q | + | + |
| 1387/1388 | R581V | + | |
| 1389/1390 | L968P | ++ | |
| 1391/1392 | A755Y | + | |
| 1393/1394 | S916Q | ++ | + |
| 1399/1400 | F698Y | + | ++ |
| 1405/1406 | L1114A | + | |

TIS reduction was measured as a reduction in the number of counts compared to the reference polypeptide of SEQ ID NO: 1200 and are defined as follows: “+” 1-10, “++” >10, “+++” >20, “++++” >50.
IHC reduction was measured as a reduction in the number of counts compared to the reference polypeptide of SEQ ID NO: 1200 and are defined as follows: “+” 1-3, “++” >3, “+++” >5, “++++” >8, “+++++” >15

TABLE 12.7
Reduction of Total Immunogenicity Score (TIS) and Immunogenic
Hit Count (IHC) for FVIII Variants Relative to SEQ ID NO: 1514

| | Amino Acid | | |
|---|---|---|---|
| SEQ ID | Differences | TIS reduction | IHC reduction |
| NO: | (Relative to | Relative to | Relative to |
| (nt/aa) | SEQ ID NO: 1514) | SEQ ID NO: 1514 | SEQ ID NO: 1514 |
| 1513/1514 | | | |
| 1609/1610 | I215E | ++++ | + |
| 2069/2070 | L318G | ++++ | + |
| 1611/1612 | I215P | ++++ | + |
| 2033/2034 | V536K/F555G/G619E/Y737K/K799Q/E809G/ | +++ | + |
| | S839K/S916T/Q921R | | |
| 2003/2004 | I1050E | +++ | + |
| 1593/1594 | M1051H | +++ | + |
| 1595/1596 | I1283H | +++ | + |
| 1597/1598 | I1283T | +++ | + |
| 1613/1614 | I215S | +++ | + |
| 1599/1600 | Y1281Q | +++ | + |
| 1601/1602 | Y1281P | +++ | + |
| 1603/1604 | S916E | +++ | + |
| 1857/1858 | E786D/R821Q | +++ | + |
| 1589/1590 | R1042E | +++ | + |
| 1615/1616 | L178C | +++ | + |
| 1869/1870 | L178W | +++ | + |
| 1955/1956 | V536P/F555E/G619E/W732K/K799S/Y805Q | +++ | + |
| 1963/1964 | V536P/F555E/G619E/W732K/K799S | +++ | + |
| 1617/1618 | I1050T | ++ | + |
| 1619/1620 | K1210T | ++ | + |
| 1999/2000 | N616D | ++ | + |
| 2005/2006 | I610H | ++ | + |
| 2025/2026 | Y1281S | ++ | + |
| 1591/1592 | Y1041N | ++ | + |
| 1657/1658 | Y175G | ++ | + |
| 1971/1972 | V536P/F555E/G619E | ++ | + |
| 2007/2008 | Q611D | ++ | + |
| 2027/2028 | A618E | ++ | + |
| 1605/1606 | M1051W | ++ | + |
| 1627/1628 | P615D | ++ | + |
| 1825/1826 | Y1104E | ++ | |
| 1989/1990 | V536P/G619E/K799S/Y805Q | ++ | + |
| 1621/1622 | Y1281K | ++ | + |
| 1853/1854 | L1138Q | ++ | |
| 1973/1974 | G619E/S839K | ++ | + |
| 1985/1986 | V536P/G619E/D801G | ++ | + |
| 1993/1994 | Y1281H | ++ | + |
| 1623/1624 | R1042A | ++ | + |
| 1749/1750 | L178Q | ++ | + |
| 1797/1798 | Y175A | ++ | + |
| 1939/1940 | K1093P | ++ | |
| 1629/1630 | I610S | ++ | + |
| 1659/1660 | K1093W | ++ | |
| 1661/1662 | F1088G | ++ | |
| 1663/1664 | K1093T | ++ | |
| 2009/2010 | R1042T | ++ | + |
| 1607/1608 | L187C | ++ | + |
| 1631/1632 | I610K | ++ | + |
| 1633/1634 | I610T | ++ | + |
| 1675/1676 | S700E | ++ | + |
| 1697/1698 | K1093V | ++ | |
| 1907/1908 | K1093N | ++ | |
| 2023/2024 | R1158N | ++ | + |
| 1625/1626 | V104S | ++ | + |
| 1739/1740 | D790P | ++ | |
| 1881/1882 | L187G | ++ | + |
| 1635/1636 | V536P | ++ | + |
| 1637/1638 | A618Y | ++ | + |
| 1645/1646 | M110H | ++ | + |
| 1733/1734 | F1088S | ++ | |
| 1871/1872 | Y1104C | ++ | |
| 1925/1926 | D790G | ++ | |
| 1977/1978 | V536P/D801G/Y805Q | ++ | + |
| 2015/2016 | T370A/L614A | ++ | + |
| 2019/2020 | K1210V | ++ | + |
| 2021/2022 | L614T | ++ | + |
| 2065/2066 | S839K | ++ | + |
| 1639/1640 | Q621W | + | + |
| 1819/1820 | Y175H | + | + |
| 1877/1878 | K1093S | + | |
| 1991/1992 | F555E/G619E/W732K/K799S/D801G/Y805Q | + | + |
| 2031/2032 | V536P/G619E/E809G/E1109G | + | + |
| 2037/2038 | V536R/G619E/G681N/K799Q/S916T/E1109G | + | + |
| 2049/2050 | G619E/G681N/S916T | + | + |
| 1693/1694 | I756G | + | |
| 1777/1778 | M1098G | + | |
| 2001/2002 | Q621K | + | + |
| 1669/1670 | T535N | + | + |
| 1723/1724 | D790W | + | |
| 1771/1772 | K1093G | + | |
| 1781/1782 | I756T | + | |
| 1959/1960 | F555E/G619E/K799S | + | + |
| 1969/1970 | A246D/G619E/K799S/D801G | + | + |
| 1979/1980 | F555E/G619E/K799S/Y805Q | + | + |
| 2011/2012 | N616Q | + | + |
| 2041/2042 | F555E/G619E/G681N/Y737K/K799Q | + | + |
| 2051/2052 | F555G/G619E/G681N/K799S | + | + |
| 1653/1654 | E786C | + | |
| 1805/1806 | Q580S | + | |
| 1909/1910 | A119G | + | |
| 1919/1920 | H712Q | + | |
| 1923/1924 | H712G | + | |
| 1957/1958 | F555E/G619E/D801G | + | + |
| 1983/1984 | F555E/G619E | + | + |
| 2063/2064 | F555G/S916T | + | |
| 1705/1706 | L178V | + | + |
| 1713/1714 | K1093A | + | |
| 1719/1720 | E1452L | + | |
| 1745/1746 | S304G | + | + |
| 1761/1762 | Y917G | + | |
| 1793/1794 | S700A | + | + |
| 1961/1962 | G619E/K799S/D801G/Y805Q | + | + |
| 1975/1976 | G619E/K799S/Y805Q | + | + |
| 1997/1998 | L614H | + | + |
| 2017/2018 | H1156D | + | + |
| 2057/2058 | G619E/K799S | + | + |
| 1641/1642 | A618P | + | + |
| 1689/1690 | T535G | + | + |
| 1753/1754 | K752G | + | |
| 1755/1756 | F1108W | + | |
| 1783/1784 | M948S | + | |
| 1785/1786 | A119V | + | |
| 1791/1792 | M1113F | + | |
| 1795/1796 | E786D | + | |
| 1931/1932 | S1084N | + | |
| 1981/1982 | G619E/D801G/Y805Q | + | + |
| 2043/2044 | G619E/G681N/K799S/S916T/E1109G | + | + |
| 2045/2046 | G619E/G681N | + | + |
| 2059/2060 | G619E | + | + |
| 1643/1644 | L614F | + | + |
| 1727/1728 | G292D | + | + |
| 1747/1748 | S1116G | + | |
| 1787/1788 | S1116L | + | |
| 1813/1814 | L1100S | + | |
| 1905/1906 | K752E | + | |
| 2013/2014 | L614N | + | + |
| 1647/1648 | R301K | + | + |
| 1867/1868 | S304E | + | + |
| 1941/1942 | K1093Y | + | |
| 2029/2030 | F555E/G681N/Y737K/K799Q | + | |
| 1709/1710 | I310G | + | |
| 1711/1712 | S700Q | + | + |
| 1759/1760 | T281A | + | |
| 1809/1810 | D731I | + | |
| 1879/1880 | H712S | + | |
| 1929/1930 | D731L | + | |
| 1949/1950 | H712T | + | |
| 2053/2054 | F555E | + | |
| 2055/2056 | W732K | + | |
| 1649/1650 | I1078S | + | |
| 1665/1666 | L650M | + | |
| 1725/1726 | D790I | + | |
| 1731/1732 | F1108M | + | |
| 1751/1752 | S1116A | + | |
| 1845/1846 | T686W | + | |
| 1859/1860 | T535C | + | + |
| 1897/1898 | D790A | + | |
| 1953/1954 | K1093R | + | |
| 1965/1966 | K799S/Y805Q | + | |
| 2039/2040 | F555G/G619E/G681N/E807R/E1109G | + | + |
| 2067/2068 | K799Q | + | |

TIS reduction was measured as a reduction in the number of counts compared to the reference polypeptide of SEQ ID NO: 1514 and are defined as follows: “+” 1-10, “++” >10, “+++” >20, “++++” >30.
IHC reduction was measured as a reduction in the number of counts compared to the reference polypeptide of SEQ ID NO: 1514 and are defined as follows: “+” 1-3, “++” >3, “+++” >5

TABLE 12.8
Reduction of Total Immunogenicity Score (TIS) and Immungenic Hit
Count (IHC) for FVIII Variants Relative to SEQ ID NO: 2072

| | Amino Acid | | IHC |
|---|---|---|---|
| SEQ ID | Differences | TIS reduction (rel | reduction (Rel |
| NO: | (Relative to | to SEQ ID NO | to SEQ ID |
| (nt/aa) | SEQ ID NO: 2072) | 2072 = 1998) | NO 2072) |
| 2097/2098 | A167G/L178Q/S700E/K752E/K1093G | +++ | +++ |
| 2101/2102 | A167G/S700E/E738Y/E739G/K1093G | ++ | ++ |
| 2103/2104 | A167G/S700E/E738Y/K752E/K1093G | +++ | ++ |
| 2105/2106 | A167G/S700E/E738Y/K752E/Y805Q/D808S/E809G/ | +++ | ++ |
| | K1093A/S1116I | | |
| 2121/2122 | E738Y/E739G/E809G/K1093G/S1116I | ++ | |
| 2137/2138 | E738Y/K752E/K1093G | ++ | |
| 2141/2142 | E738Y/K799S/E809G/K1093G | ++ | |
| 2189/2190 | G681N/E738Y/E739G/K1093G | ++ | |
| 2203/2204 | G681N/K752E/K1093G | ++ | |
| 2205/2206 | G681N/S700E | ++ | ++ |
| 2209/2210 | G681N/S700E/E809G | ++ | ++ |
| 2211/2212 | G681N/S700E/K1093G | +++ | ++ |
| 2213/2214 | G681N/S700E/K752E/D808S/E809G/K1093G/S1116I | +++ | ++ |
| 2215/2216 | G681N/S700E/K752E/K1093G | +++ | ++ |
| 2219/2220 | G681N/S700E/Y805Q/E809G | ++ | ++ |
| 2225/2226 | K1093G/S1116I | ++ | |
| 2227/2228 | K752E | + | |
| 2233/2234 | K752E/E800V/K1093G | + | |
| 2235/2236 | K752E/E809G | + | |
| 2241/2242 | K752E/S1116I | + | |
| 2249/2250 | L178Q/G681N | ++ | + |
| 2251/2252 | L178Q/G681N/E738Y/S1116I | ++ | + |
| 2253/2254 | L178Q/K752E/E809G | ++ | + |
| 2265/2266 | S543P/E738Y/E809G/K1093G | ++ | |
| 2267/2268 | S543P/E739G/K1093A | ++ | |
| 2271/2272 | S543P/E809G/K1093G | ++ | |
| 2275/2276 | S543P/G681N/E738Y/E739G/K752E/E809G/K1093G | ++ | |
| 2277/2278 | S543P/G681N/E738Y/E739G/K752E/K1093G | ++ | |
| 2279/2280 | S543P/G681N/E739G/K752E/E800V/Y805Q/E809G/ | + | |
| | K1093G | | |
| 2281/2282 | S543P/G681N/S700E/E738Y/E739G/E800V/K1093G/ | ++ | ++ |
| | S1116I | | |
| 2283/2284 | S543P/G681N/S700E/E738Y/E739G/Y805Q/D808S/ | ++ | ++ |
| | E809G | | |
| 2287/2288 | S543P/G681N/S700E/K752E/E809G/K1093G | +++ | ++ |
| 2289/2290 | S543P/K1093G | ++ | |
| 2301/2302 | S543P/S700E/K752E/E800V/K1093G | ++ | ++ |
| 2305/2306 | S543P/S700E/K752E/K1093G | +++ | ++ |
| 2307/2308 | S700E | ++ | ++ |
| 2309/2310 | S700E/E738Y/E739G/E800V/K1093G | ++ | ++ |
| 2313/2314 | S700E/E738Y/E800V/K1093G | ++ | ++ |
| 2315/2316 | S700E/E738Y/S1116I | ++ | ++ |
| 2317/2318 | S700E/E809G | ++ | ++ |
| 2319/2320 | S700E/E809G/K1093G | +++ | ++ |
| 2321/2322 | S700E/K1093G | +++ | ++ |
| 2323/2324 | S700E/K1093G/S1116I | +++ | ++ |
| 2325/2326 | S700E/K752E | ++ | ++ |
| 2329/2330 | S700E/K752E/E809G/K1093G | +++ | ++ |
| 2333/2334 | S700E/K752E/Y805Q/E809G/K1093A | +++ | ++ |
| 2335/2336 | S700E/K799S/E800V | ++ | ++ |
| 2337/2338 | S700E/S1116I | ++ | ++ |
| 2339/2340 | S700E/Y805Q/D808S/E809G/E1076S | ++ | + |

TIS reduction was measured as a reduction in the number of counts compared to the reference polypeptide of SEQ ID NO: 2072 and are defined as follows: “+” 1-4, “++” 5-20, “+++” >20.
IHC reduction was measured as a reduction in the number of counts compared to the reference polypeptide of SEQ ID NO: 2072 and are defined as follows: “+” 4, “++” 5, “+++” >5

### Example 13

### Peptide T Cell Assay

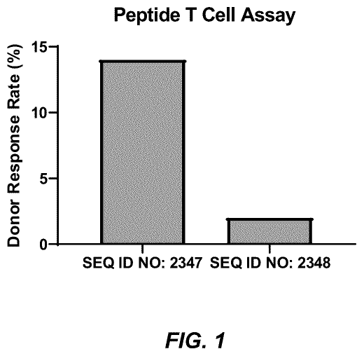

In this Example, peptides were designed based on the wild-type B-domain deleted human factor VIII with or without mutations thought to alter the immunogenicity of the protein. Specifically, two polypeptides (SEQ ID NO: 2347 and SEQ ID NO: 2348) were prepared by Abzena Ltd. (Cambridge, United Kingdom). The sequence of the peptide of SEQ ID NO: 2347 has no amino acid changes compared to the sequence in SEQ ID NO: 2 while the sequence of the peptide of SEQ ID NO: 2348 contains a L→G substitution at amino acid position 12 of the peptide, which is equivalent to a L318G substitution relative to SEQ ID NO: 2. These peptides were analyzed for the presence of CD4⁺ T cell epitopes using EpiScreen™ T cell epitope matching technology, using peripheral blood mononuclear cells (PBMCs) from 50 healthy human donors. PBMCs were adjusted to a cell density of 2.5-3.0E6 cells/mL using AIM V© culture medium (Invitrogen, Paisley, UK). 5 μM of the peptides, or control medium, was added to the PBMCs in a flat bottomed 96-well plate. For each peptide-donor pair, 6 reactions were performed. Cultures were incubated for 6 days before adding 0.75 μCi of tritium-labeled thymidine (Perkin Elmer©, Beaconsfield, UK) to each well. Cultures were incubated another 18 hours before harvesting the cells onto filter mats using a TomTec Mach III cell harvester (Receptor Tech. Ltd., Warwick, UK). Counts per minute (CPM) for each well were determined Meltilex™ (Perkin Elmer®, Beaconsfield, UK) scintillation counting on a Microplate Beta Counter (Perkin Elmer®, Beaconsfield, UK) in paralux, low background counting mode. A Student's t-test comparing the response of the wells receiving peptide to the wells receiving medium was performed to determine the significance of the response (p<0.05). Positive responses were those whose mean CPM value of an individual peptide-donor pair divided by the average CPM value of the medium controls was above 1.90. The results for this experiment are shown in FIG. 1.

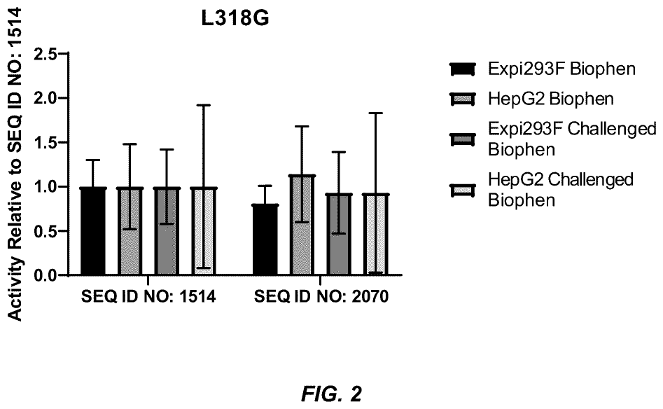

Similar to Example 1, directed evolution techniques were used to generate a gene variant containing the mutation tested using the Abzena, Ltd. Peptide T Cell Assay (SEQ ID NO: 2069 (nucleotide sequence); SEQ ID NO: 2070 (amino acid sequence)) derived from SEQ ID NO: 1514 within the same plasmid construct (See e.g., U.S. Pat. No. 8,383,346 and WO2010/144103). This variant (SEQ ID NO: 2070) was produced using the methods described in Example 2 and tested for FVIII activity after no incubation and after incubation in Hemophilia A patient plasma (George King Bio-Medical, Inc.), as described in Example 3. The variant was tested using the chromogenic assay, as described in Example 3. The results are shown in FIG. 2.

### Example 14

### Activity of Truncated FVIII Variants Derived from SEQ ID NO: 2

Similar to Example 1, directed evolution techniques generally known by those skilled in the art were used to generate gene variants derived from SEQ ID NO: 2 containing truncated versions of factor VIII within the same plasmid construct (See e.g., U.S. Pat. No. 8,383,346 and WO2010/144103). These variants were produced using the methods described in Example 2 and tested using the chromogenic assays, as described in Example 3. The results are shown in FIG. 3.

### Example 15

### Truncated FVIII Variants Derived from SEQ ID NO: 2

In this Example, experiments conducted to determine FVIII variant activity by assaying the protein activity are described. These variants were tested for FVIII activity using the chromogenic assay, as described in Example 3. Results are shown in Table 15.1 for variants expressed using Expi293F cells.

TABLE 15.1
Activity of FVIII Variants Relative to SEQ ID NO: 2

| | Amino Acid | |
|---|---|---|
| SEQ ID | Differences | Expi293F Biophen |
| NO: | (Relative to | FIOP (Primary) |
| (nt/aa) | SEQ ID NO: 2) | Relative to SEQ ID NO: 2 |
| 3/4 | T1148N | + |
| 13/14 | L171Q | +++ |
| 17/18 | V63R | ++ |
| 35/36 | L1248S | ++ |
| 67/68 | L1248R | ++ |
| 85/86 | S332Q | ++ |
| 95/96 | L1248G | +++ |
| 153/154 | S332W | + |
| 157/158 | V63H | ++ |
| 185/186 | S332P | ++ |
| 193/194 | L171P | +++ |
| 245/246 | K732G | ++ |
| 251/252 | L1377D | ++++ |
| 261/262 | E306G | +++ |
| 265/266 | L1377G | +++ |
| 267/268 | S332F | + |
| 269/270 | L1377E | ++++ |
| 281/282 | L1377R | +++ |
| 283/284 | L1377S | ++++ |
| 287/288 | L171M | +++ |
| 291/292 | L1248D | ++ |
| 293/294 | E306S | + |
| 297/298 | L171H | +++ |
| 299/300 | L1377A | +++ |
| 301/302 | L1377V | +++ |
| 305/306 | T1148R | ++ |
| 2359/2360 | E306T | + |
| 2361/2362 | E809L | + |
| 2363/2364 | E800L | + |
| 2365/2366 | F387A | + |
| 2367/2368 | Y738A | + |
| 2369/2370 | I499C | + |
| 2371/2372 | M681R | + |
| 2373/2374 | K1093E | + |
| 2375/2376 | T1148A | + |
| 2377/2378 | V63W | + |
| 2379/2380 | K752Q | + |
| 2381/2382 | I390E | + |
| 2383/2384 | V534M | + |
| 2385/2386 | L749P | + |
| 2387/2388 | K1093M | + |
| 2389/2390 | V1439S | + |
| 2391/2392 | S260K | + |
| 2393/2394 | M558V | + |
| 2395/2396 | S332N | + |
| 2397/2398 | I390T | + |
| 2399/2400 | Y738D | + |
| 2401/2402 | S700V | + |
| 2403/2404 | S1414P | + |
| 2405/2406 | K1093P | + |
| 2407/2408 | L749D | + |
| 2409/2410 | H212Y | + |
| 2411/2412 | M681C | + |
| 2413/2414 | Y805R | + |
| 2415/2416 | T1148S | + |
| 2417/2418 | E800R | + |
| 2419/2420 | S332V | + |
| 2421/2422 | L171K | + |
| 2423/2424 | E809Q | + |
| 2425/2426 | M681L | + |
| 2427/2428 | E800G | + |
| 2429/2430 | Y738T | + |
| 2431/2432 | M681E | + |
| 2433/2434 | K732C | + |
| 2435/2436 | L749C | + |
| 2437/2438 | S1414T | + |
| 2439/2440 | V63C | + |
| 2441/2442 | L171F | + |
| 2443/2444 | L318M | + |
| 2445/2446 | M681T | + |
| 2447/2448 | V534F | + |
| 2449/2450 | L750K | + |
| 2451/2452 | Y738N | + |
| 2453/2454 | K1093V | + |
| 2455/2456 | L749Q | + |
| 2457/2458 | H212F | + |
| 2459/2460 | H212M | + |
| 2461/2462 | I775S | + |
| 2463/2464 | F387W | + |
| 2465/2466 | I499L | + |
| 2467/2468 | F387R | + |
| 2469/2470 | T1148L | + |
| 2471/2472 | V1439T | + |
| 2473/2474 | E800M | + |
| 2475/2476 | L750R | + |
| 2477/2478 | V1439A | + |
| 2479/2480 | V63I | + |
| 2481/2482 | V393H | + |
| 2483/2484 | M558R | + |
| 2485/2486 | V63S | + |
| 2487/2488 | M558T | + |
| 2489/2490 | S332A | + |
| 2491/2492 | Y805C | + |
| 2493/2494 | L749F | + |
| 2495/2496 | H212A | + |
| 2497/2498 | L749A | + |
| 2499/2500 | K752L | + |
| 2501/2502 | L1248I | + |
| 2503/2504 | M681A | + |
| 2505/2506 | L171R | + |
| 2507/2508 | I775N | + |
| 2509/2510 | F387L | + |
| 2511/2512 | E809A | + |
| 2513/2514 | Y805G | + |
| 2515/2516 | K752R | + |
| 2517/2518 | M320I | + |
| 2519/2520 | V63E | + |
| 2521/2522 | Y805H | + |
| 2523/2524 | L749Y | + |
| 2525/2526 | S332G | + |
| 2527/2528 | Y805K | + |
| 2529/2530 | M558S | + |
| 2531/2532 | E800V | + |
| 2533/2534 | L1377M | + |
| 2535/2536 | E800W | + |
| 2537/2538 | E809C | + |
| 2539/2540 | Y738E | + |
| 2541/2542 | V1439H | + |
| 2543/2544 | L318V | + |
| 2545/2546 | E800Y | + |
| 2547/2548 | H212V | + |
| 2549/2550 | H212N | + |
| 2551/2552 | E800Q | + |
| 2553/2554 | H212K | + |
| 2555/2556 | L749M | + |
| 2557/2558 | S1414H | + |
| 2559/2560 | L1248C | ++ |
| 2561/2562 | L749K | ++ |
| 2563/2564 | M681Q | ++ |
| 2565/2566 | V393Y | ++ |
| 2567/2568 | Y738H | ++ |
| 2569/2570 | L750S | ++ |
| 2571/2572 | K732L | ++ |
| 2573/2574 | K752F | ++ |
| 2575/2576 | K1093S | ++ |
| 2577/2578 | L750T | ++ |
| 2579/2580 | E800I | ++ |
| 2581/2582 | E809R | ++ |
| 2583/2584 | S1414W | ++ |
| 2585/2586 | M558L | ++ |
| 2587/2588 | L318Q | ++ |
| 2589/2590 | V393Q | ++ |
| 2591/2592 | T1148E | ++ |
| 2593/2594 | L1248F | ++ |
| 2595/2596 | H212R | ++ |
| 2597/2598 | H212P | ++ |
| 2599/2600 | M681N | ++ |
| 2601/2602 | L749T | ++ |
| 2603/2604 | L171N | ++ |
| 2605/2606 | E800C | ++ |
| 2607/2608 | E809K | ++ |
| 2609/2610 | S260T | ++ |
| 2611/2612 | S332R | ++ |
| 2613/2614 | L750N | ++ |
| 2615/2616 | V534L | ++ |
| 2617/2618 | I390P | ++ |
| 2619/2620 | Y805S | ++ |
| 2621/2622 | M320Q | ++ |
| 2623/2624 | K732T | ++ |
| 2625/2626 | Y738S | ++ |
| 2627/2628 | V63K | ++ |
| 2629/2630 | E800N | ++ |
| 2631/2632 | L1248M | ++ |
| 2633/2634 | Y805L | ++ |
| 2635/2636 | E306A | ++ |
| 2637/2638 | S1414C | ++ |
| 2639/2640 | Y805P | ++ |
| 2641/2642 | H212L | ++ |
| 2643/2644 | L750G | ++ |
| 2645/2646 | K752M | ++ |
| 2647/2648 | S700E | ++ |
| 2649/2650 | S332Y | ++ |
| 2651/2652 | I390V | ++ |
| 2653/2654 | M681G | ++ |
| 2655/2656 | M558Q | ++ |
| 2657/2658 | V63F | ++ |
| 2659/2660 | E809W | ++ |
| 2661/2662 | M558E | ++ |
| 2663/2664 | M558G | ++ |
| 2665/2666 | L749I | ++ |
| 2667/2668 | K732Q | ++ |
| 2669/2670 | Y805M | ++ |
| 2671/2672 | I775K | ++ |
| 2673/2674 | F387T | ++ |
| 2675/2676 | L750Q | ++ |
| 2677/2678 | I390L | ++ |
| 2679/2680 | K732V | ++ |
| 2681/2682 | K752D | ++ |
| 2683/2684 | K752S | ++ |
| 2685/2686 | K732D | ++ |
| 2687/2688 | T1148C | ++ |
| 2689/2690 | M558P | ++ |
| 2691/2692 | E809I | ++ |
| 2693/2694 | L750W | ++ |
| 2695/2696 | S332H | ++ |
| 2697/2698 | E800P | ++ |
| 2699/2700 | K732R | ++ |
| 2701/2702 | L1248W | ++ |
| 2703/2704 | K752E | ++ |
| 2705/2706 | V63Y | ++ |
| 2707/2708 | V63T | ++ |
| 2709/2710 | L749G | ++ |
| 2711/2712 | I775G | ++ |
| 2713/2714 | K1093T | ++ |
| 2715/2716 | E306P | ++ |
| 2717/2718 | V393R | ++ |
| 2719/2720 | E809N | ++ |
| 2721/2722 | E809S | ++ |
| 2723/2724 | M681F | ++ |
| 2725/2726 | K732S | ++ |
| 2727/2728 | L750V | ++ |
| 2729/2730 | S1414A | ++ |
| 2731/2732 | E809T | ++ |
| 2733/2734 | K1093Q | ++ |
| 2735/2736 | L1248E | ++ |
| 2737/2738 | L171W | ++ |
| 2739/2740 | K732Y | ++ |
| 2741/2742 | L171T | ++ |
| 2743/2744 | Y805W | ++ |
| 2745/2746 | E809G | ++ |
| 2747/2748 | F387V | ++ |
| 2749/2750 | H212S | ++ |
| 2751/2752 | T1148M | ++ |
| 2753/2754 | I775P | ++ |
| 2755/2756 | K732M | ++ |
| 2757/2758 | V63L | ++ |
| 2759/2760 | L750P | ++ |
| 2761/2762 | V393F | ++ |
| 2763/2764 | M320P | ++ |
| 2765/2766 | V393G | ++ |
| 2767/2768 | S700T | ++ |
| 2769/2770 | K752V | ++ |
| 2771/2772 | E809V | ++ |
| 2773/2774 | K1093A | ++ |
| 2775/2776 | M320G | ++ |
| 2777/2778 | I390M | ++ |
| 2779/2780 | L1377I | ++ |
| 2781/2782 | M681W | ++ |
| 2783/2784 | S1414G | ++ |
| 2785/2786 | L750M | ++ |
| 2787/2788 | E800T | ++ |
| 2789/2790 | L318T | ++ |
| 2791/2792 | L171Y | ++ |
| 2793/2794 | M558W | ++ |
| 2795/2796 | Y805Q | ++ |
| 2797/2798 | M558A | ++ |
| 2799/2800 | M320S | ++ |
| 2801/2802 | L750D | ++ |
| 2803/2804 | I775V | ++ |
| 2805/2806 | S700A | ++ |
| 2807/2808 | L1377Y | ++ |
| 2809/2810 | M320L | +++ |
| 2811/2812 | L318R | +++ |
| 2813/2814 | V393A | +++ |
| 2815/2816 | L171V | +++ |
| 2817/2818 | V1439E | +++ |
| 2819/2820 | L1248Y | +++ |
| 2821/2822 | S332T | +++ |
| 2823/2824 | L1248Q | +++ |
| 2825/2826 | L1377C | +++ |
| 2827/2828 | L1248H | +++ |
| 2829/2830 | M681S | +++ |
| 2831/2832 | I390G | +++ |
| 2833/2834 | L171A | +++ |
| 2835/2836 | L1248T | +++ |
| 2837/2838 | V393M | +++ |
| 2839/2840 | L171S | +++ |
| 2841/2842 | E306D | +++ |
| 2843/2844 | L1248A | +++ |
| 2845/2846 | S1414E | +++ |
| 2847/2848 | S1414D | +++ |
| 2849/2850 | L1248K | +++ |
| 2851/2852 | I390S | +++ |
| 2853/2854 | L318K | +++ |
| 2855/2856 | M320T | +++ |
| 2857/2858 | L171D | +++ |
| 2859/2860 | L171E | +++ |
| 2861/2862 | M320V | +++ |
| 2863/2864 | L1377P | +++ |
| 2865/2866 | M320R | +++ |
| 2867/2868 | L1377T | +++ |
| 2869/2870 | L1377H | +++ |

All activities were determined relative to the reference polypeptide of SEQ ID NO: 2. Levels of increased activity are defined as follows: “+” 0.9 to 1.1, “++” >1.1, “+++” >1.5, “++++” >3.5

Active FVIII variants identified in screens from this Example were assessed for their levels of predicted immunogenicity by evaluating their binding to the eight common Class 11 HILA-DR alleles, as in Example 12. The total immunogenicity score and immunogenic hit count were calculated for each variant. The total immunogenicity score (TIS) reflects the overall predicted immunogenicity of the variant (i.e., a higher score indicates a higher level of predicted immunogenicity). The immunogenic “hit count” (IHC) indicates the number of 15-mer analysis frames with an unusually high potential for immunogenicity (i.e., a higher score indicates a higher potential for immunogenicity). Mutations resulting in a reduced total immunogenicity score and/or an immunogenic hit count compared to the reference sequence were considered to be potential “deimmunizing mutations” and are shown in Table 15.2.

TABLE 15.2
Reduction of Total Immunogenicity Score (TIS) and Immungenic
Hit Count (IHC) for FVIII Variants Relative to SEQ ID NO: 2

| | Amino Acid | | |
|---|---|---|---|
| | Differences | TIS reduction | IHC reduction |
| SEQ ID NO: | (Relative to | Relative to | Relative to |
| (nt/aa) | SEQ ID NO: 2) | SEQ ID NO: 2 | SEQ ID NO: 2 |
| 2365/2366 | F387A | +++ | + |
| 2369/2370 | I499C | ++ | |
| 2371/2372 | M681R | + | + |
| 2373/2374 | K1093E | ++ | |
| 2379/2380 | K752Q | ++ | + |
| 2381/2382 | I390E | +++ | + |
| 2385/2386 | L749P | ++ | + |
| 2389/2390 | V1439S | ++ | + |
| 2391/2392 | S260K | + | + |
| 2393/2394 | M558V | ++ | + |
| 2395/2396 | S332N | + | + |
| 2397/2398 | I390T | ++ | + |
| 2401/2402 | S700V | | + |
| 2405/2406 | K1093P | ++ | |
| 2407/2408 | L749D | +++ | + |
| 2411/2412 | M681C | +++ | + |
| 2419/2420 | S332V | | + |
| 2421/2422 | L171K | + | |
| 2431/2432 | M681E | +++ | + |
| 2435/2436 | L749C | +++ | + |
| 2439/2440 | V63C | +++ | + |
| 2445/2446 | M681T | +++ | + |
| 2447/2448 | V534F | + | + |
| 2449/2450 | L750K | + | + |
| 2453/2454 | K1093V | ++ | |
| 2455/2456 | L749Q | ++ | + |
| 2459/2460 | H212M | + | |
| 2463/2464 | F387W | + | + |
| 2465/2466 | 1499L | + | |
| 2467/2468 | F387R | ++ | + |
| 2471/2472 | V1439T | ++ | + |
| 2475/2476 | L750R | + | |
| 2477/2478 | V1439A | + | + |
| 2483/2484 | M558R | ++ | + |
| 2485/2486 | V63S | +++ | + |
| 2487/2488 | M558T | +++ | + |
| 2493/2494 | L749F | + | + |
| 2495/2496 | H212A | + | + |
| 2497/2498 | L749A | + | + |
| 2499/2500 | K752L | + | + |
| 2503/2504 | M681A | +++ | + |
| 2505/2506 | L171R | + | |
| 2509/2510 | F387L | + | + |
| 2517/2518 | M320I | + | + |
| 2519/2520 | V63E | +++ | + |
| 2525/2526 | S332G | + | + |
| 2529/2530 | M558S | ++ | + |
| 2533/2534 | L1377M | + | + |
| 2541/2542 | V1439H | + | + |
| 2543/2544 | L318V | ++ | + |
| 2553/2554 | H212K | + | |
| 2555/2556 | L749M | | + |
| 2559/2560 | L1248C | + | + |
| 2561/2562 | L749K | + | + |
| 2563/2564 | M681Q | +++ | + |
| 2569/2570 | L750S | ++ | + |
| 2573/2574 | K752F | + | + |
| 2575/2576 | K1093S | + | |
| 2577/2578 | L750T | + | + |
| 2585/2586 | M558L | + | + |
| 2587/2588 | L318Q | +++ | + |
| 2589/2590 | V393Q | + | |
| 2591/2592 | T1148E | + | + |
| 2593/2594 | L1248F | + | + |
| 2597/2598 | H212P | + | + |
| 2599/2600 | M681N | +++ | + |
| 2601/2602 | L749T | ++ | + |
| 2603/2604 | L171N | ++ | + |
| 2609/2610 | S260T | + | + |
| 2613/2614 | L750N | ++ | + |
| 2617/2618 | I390P | +++ | + |
| 2621/2622 | M320Q | + | + |
| 2627/2628 | V63K | ++ | + |
| 2631/2632 | L1248M | + | + |
| 2637/2638 | S1414C | + | |
| 2643/2644 | L750G | + | + |
| 2645/2646 | K752M | + | |
| 2647/2648 | S700E | ++ | + |
| 2649/2650 | S332Y | + | + |
| 2651/2652 | I390V | + | + |
| 2653/2654 | M681G | +++ | + |
| 2655/2656 | M558Q | +++ | + |
| 2661/2662 | M558E | +++ | + |
| 2663/2664 | M558G | +++ | + |
| 2671/2672 | I775K | + | |
| 2673/2674 | F387T | +++ | + |
| 2675/2676 | L750Q | + | + |
| 2681/2682 | K752D | ++ | + |
| 2683/2684 | K752S | + | + |
| 2687/2688 | T1148C | + | + |
| 2689/2690 | M558P | +++ | + |
| 2693/2694 | L750W | + | + |
| 2701/2702 | L1248W | | + |
| 2703/2704 | K752E | ++ | + |
| 2707/2708 | V63T | +++ | + |
| 2709/2710 | L749G | +++ | + |
| 2711/2712 | I775G | + | |
| 2713/2714 | K1093T | ++ | |
| 2727/2728 | L750V | | + |
| 2733/2734 | K1093Q | ++ | |
| 2735/2736 | L1248E | + | + |
| 2737/2738 | L171W | + | |
| 2741/2742 | L171T | + | |
| 2747/2748 | F387V | ++ | + |
| 2749/2750 | H212S | + | + |
| 2753/2754 | I775P | + | |
| 2759/2760 | L750P | ++ | + |
| 2761/2762 | V393F | + | |
| 2763/2764 | M320P | ++ | + |
| 2765/2766 | V393G | +++ | + |
| 2767/2768 | S700T | + | + |
| 2769/2770 | K752V | ++ | + |
| 2773/2774 | K1093A | + | |
| 2775/2776 | M320G | ++ | + |
| 2777/2778 | I390M | + | |
| 2779/2780 | L1377I | + | + |
| 2781/2782 | M681W | + | + |
| 2783/2784 | S1414G | + | |
| 2789/2790 | L318T | ++ | |
| 2793/2794 | M558W | ++ | + |
| 2797/2798 | M558A | +++ | + |
| 2799/2800 | M320S | + | |
| 2801/2802 | L750D | +++ | + |
| 2803/2804 | I775V | + | |
| 2805/2806 | S700A | + | + |
| 2807/2808 | L1377Y | ++ | + |
| 2809/2810 | M320L | + | + |
| 2817/2818 | V1439E | +++ | + |
| 2819/2820 | L1248Y | + | + |
| 2823/2824 | L1248Q | + | + |
| 2825/2826 | L1377C | ++ | + |
| 2827/2828 | L1248H | + | + |
| 2829/2830 | M681S | +++ | + |
| 2831/2832 | I390G | +++ | + |
| 2833/2834 | L171A | + | + |
| 2835/2836 | L1248T | + | + |
| 2839/2840 | L171S | + | + |
| 2841/2842 | E306D | + | |
| 2843/2844 | L1248A | + | + |
| 2847/2848 | S1414D | + | |
| 2849/2850 | L1248K | ++ | + |
| 2851/2852 | I390S | + | + |
| 2853/2854 | L318K | ++ | + |
| 2855/2856 | M320T | ++ | + |
| 2857/2858 | L171D | ++ | + |
| 2859/2860 | L171E | ++ | + |
| 2861/2862 | M320V | + | + |
| 2863/2864 | L1377P | ++ | + |
| 2865/2866 | M320R | | + |
| 2867/2868 | L1377T | ++ | + |
| 2869/2870 | L1377H | ++ | + |

TIS reduction was measured as a reduction in the number of counts compared to the reference polypeptide of SEQ ID NO: 2 and are defined as follows: “+” 1-10, “++” >10, “+++” >20.
IHC reduction was measured as a reduction in the number of counts compared to the reference polypeptide of SEQ ID NO: 2 and are defined as follows: “+” 1-3, “++” >3, “+++” >5

### Example 16

### In Vitro Characterization of FVIII Variants

In this Example, experiments conducted to characterize FVIII variants produced as indicated herein are described.

Production of Factor VIII in Expi293F™ Cells or HepG2 cells

Expi293F™ (ThermoFisher Scientific) cells or HepG2 cells were transfected with pcDNA 3.1(+) or a CMV promoter and BGH-pA containing vectors encoding wild type FVIII with B-domain deleted, or variants, using the lipofection method with ExpiFectamine™ 293 Transfection Kit (ThermoFisher Scientific) or LIPOFECTAMINE® 3000 Reagent (ThermoFisher Scientific). Expi293F™ (ThermoFisher Scientific) cells were cultured in growth medium (Expi293™ Expression Medium; ThermoFisher Scientific). HepG2 cells were cultured in growth medium (EMEM with 10% fetal bovine serum, both from Corning). Expi293F™ cells or HepG2 cells were transfected with plasmid DNA and grown in suspension for 3-7 days. Conditioned media was then harvested, clarified by centrifugation and filtration, and stored at −80° C. until characterization.

Analysis of FVIII Variant Expression from Expi293F™ Cells or HepG2 Cells (ELISA)

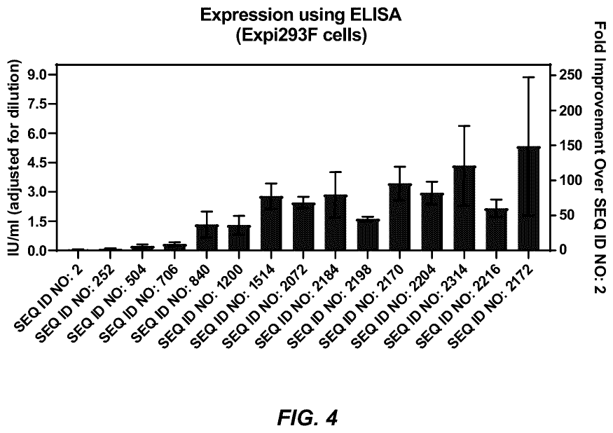

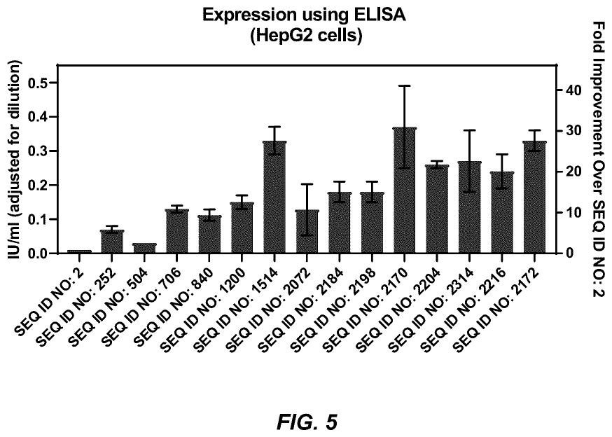

FVIII variant expression in conditioned media was analyzed using human factor VIII ELISA kit (Ab272771, Abcam) according to the company's standard operating protocol. The colorimetric assay measures total human FVIII in the 0.0002 IU/ml-0.84 IU/ml range. FVIII variants above 0.84 IU/ml were diluted in tris-BSA buffer (A221406-RUO, Aniara Diagnostica) in a 96-well microtiter plate. The absorbance of all wells was measured at 450 nm using the EnVision Multimode Plate Reader. The results from this analysis are presented in FIG. 4 & FIG. 5, plotting mean±standard deviation of triplicates.

### Analysis of FVIII Variant Activity (Chromogenic Assay)

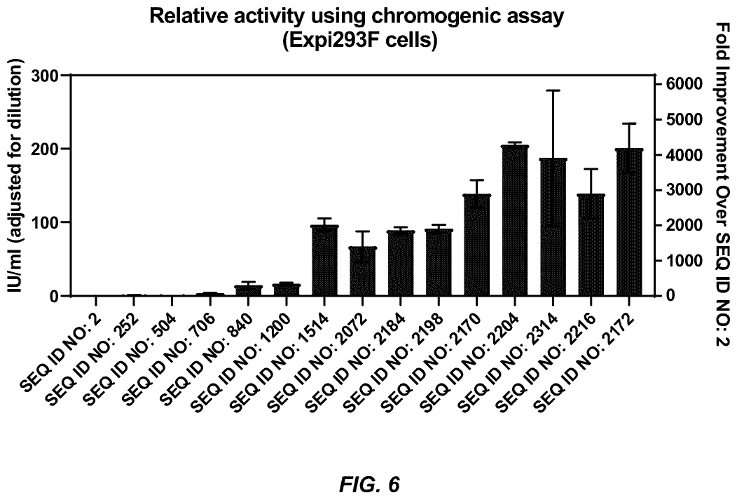

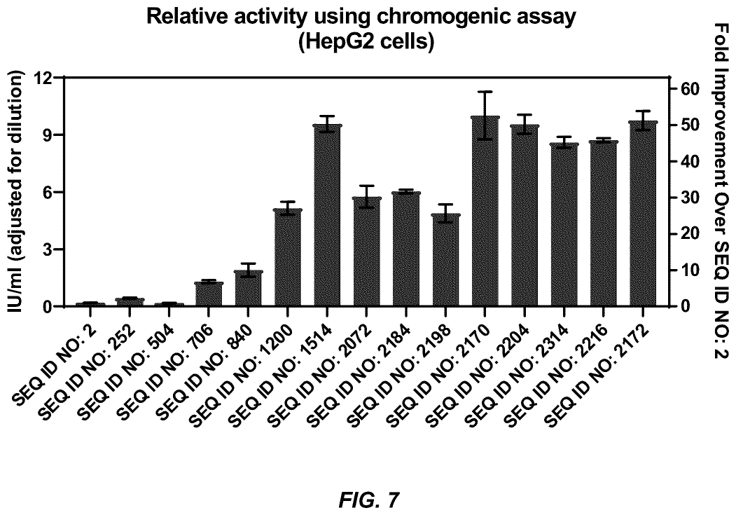

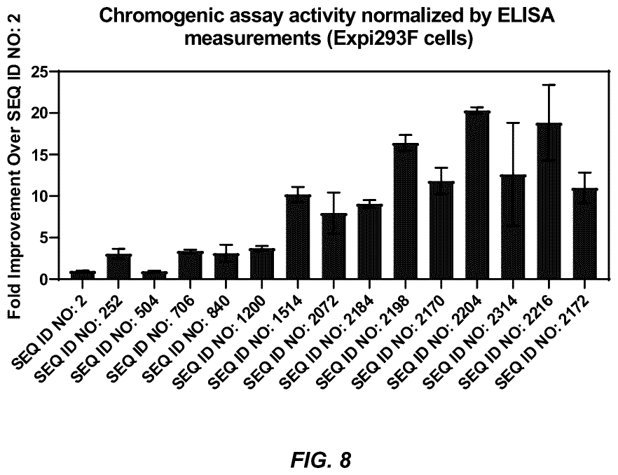

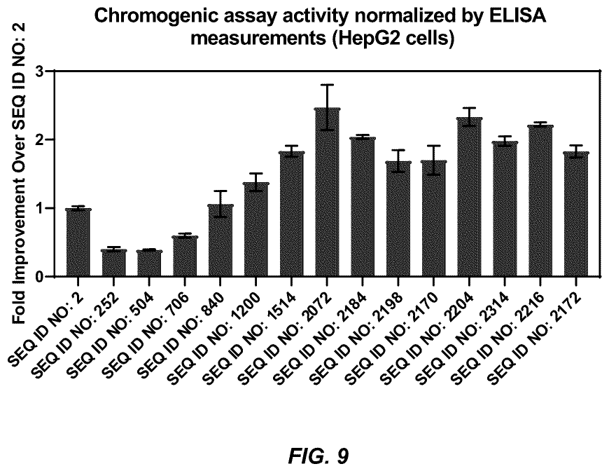

FVIII variant activity was determined by measuring the release of paranitroaniline (pNa) from a synthetic peptide substrate. For an unchallenged assay, 25 μL of diluted or undiluted Expi293F or HepG2 conditioned media produced as described above were warmed to 37° C. and mixed with 25 μL of “Reagent 1” (BIOPHEN™ FVIII:C, Aniara) and 25 μL of “Reagent 2” (BIOPHEN™ FVIII:C, Aniara) in a 96-well, black, opaque bottom microtiter plate. The reactions were mixed briefly and incubated at 37° C. for 5 minutes. 25 μL of “Reagent 3” (BIOPHEN™ FVIII:C, Aniara) was then added, and the reactions were mixed briefly and incubated at 37° C. for 5 minutes prior to quenching with 25 μL pf 2% citric acid. Release of pNA was analyzed using an EnVision® 2105 Multimode Plate Reader (PerkinElmer) monitoring absorbance at 405 nm. “Reagent 1”, “Reagent 2”, and “Reagent 3” are components of a chromogenic assay (BIOPHEN™ FVIII:C) supplied by Aniara and used as recommended by the supplier. The results from this analysis are presented in FIG. 6 & FIG. 7 (before normalization) and FIG. 8 & FIG. 9 (after normalization using the protein concentration determined by ELISA above), plotting mean±standard deviation of triplicates.

### Analysis of FVIII Variant Activity (Clotting Assay)

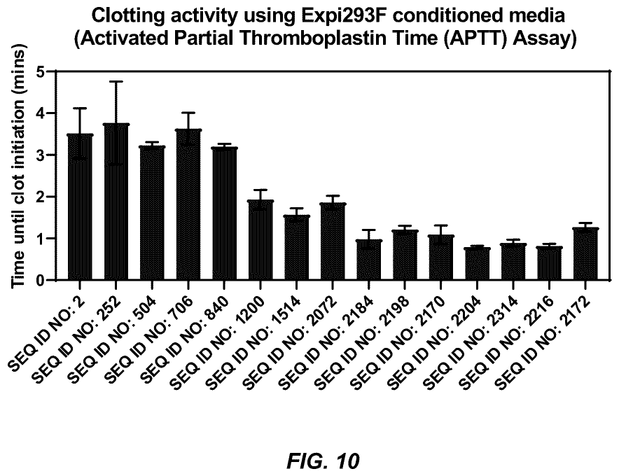

FVIII variant activity was determined by measuring the clotting rate of Hemophilia A patient plasma (George King Bio-Medical, Inc.) using the activated partial thromboplastin time assay. For an unchallenged assay, 25 μL of diluted or undiluted Expi293F or HepG2 conditioned media produced as described above were warmed to 37° C. and mixed with 30 μL of Hemophilia A patient plasma (George King Bio-Medical, Inc.) and 50 μL of aPTT-XL reagent (Pacific Hemostasis) in a 96-well, black, opaque bottom microtiter plate. The reactions were mixed briefly and incubated at 37° C. for 3 minutes. 10 μL of 200 μM calcium chloride (Sigma Aldrich) was then added, and the reactions were mixed briefly. Clotting was analyzed using an EnVision® 2105 Multimode Plate Reader (PerkinElmer) monitoring absorbance at 405 nm over time for 5-10 minutes. Analyses were performed using an R script to extrapolate the clot initiation time from the collected data points. The results from this analysis are presented in FIG. 10, plotting mean±standard deviation of triplicates.

### Analysis of FVIII Variant Stability in Hemophilia A Patient Plasma

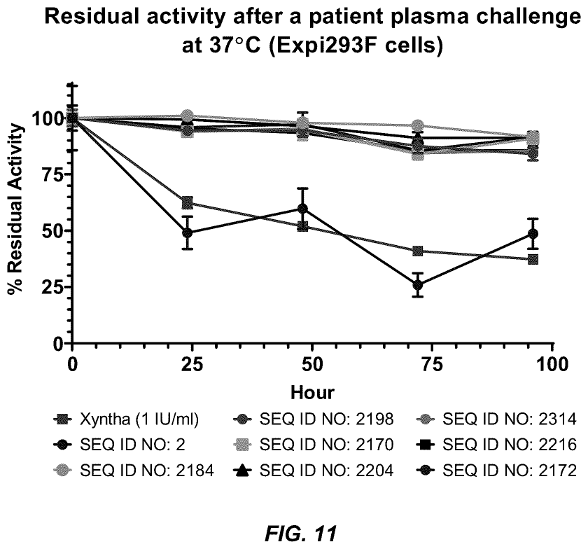

FVIII variants were challenged with Hemophilia A patient plasma (George King Bio-Medical, Inc.) to simulate the conditions that the variants may encounter within the bloodstream. First, 50 μL of Expi293F or HepG2 conditioned media produced as described above and 50 μL of Hemophilia A patient plasma (George King Bio-Medical, Inc.) were added to the wells of a 96-well round bottom microtiter plate. The plates were sealed with a PlateLoc® thermal microplate sealer (Agilent), and incubated at 37-55° C. for 1-96 h. The samples were then analyzed by the chromogenic assay described above. The results from this analysis are presented in FIG. 11, plotting mean±standard deviation of triplicates.

While the invention has been described with reference to the specific embodiments, various changes can be made and equivalents can be substituted to adapt to a particular situation, material, composition of matter, process, process step or steps, thereby achieving benefits of the invention without departing from the scope of what is claimed.

For all purposes, each and every publication and patent document cited in this disclosure is incorporated herein by reference as if each such publication or document was specifically and individually indicated to be incorporated herein by reference. Citation of publications and patent documents is not intended as an indication that any such document is pertinent prior art, nor does it constitute an admission as to its contents or date.

1. A recombinant factor VIII polypeptide, or a biologically active fragment thereof, comprising an amino acid sequence having at least 70%, 75%, 80%, 81%, 82%, 83%, 84%, 85%, 86%, 87%, 88%, 89%, 90%, 91%, 92%, 93%, 94%, 95%, 96%, 97%, 98%, or 99% or more sequence identity to a reference sequence corresponding to residues 20-1457 of SEQ ID NO: 2, 252, 504, 706, 840, 1200, 1514, or 2072, or to a reference sequence corresponding to SEQ ID NO: 2, 252, 504, 706, 840, 1200, 1514, or 2072, wherein the amino acid sequence comprises one or more substitutions relative to the reference sequence corresponding to residues 20-1457 of SEQ ID NO: 2, 252, 504, 706, 840, 1200, 1514, or 2072, or relative to the reference sequence corresponding to SEQ ID NO: 2, 252, 504, 706, 840, 1200, 1514, or 2072.

2. The recombinant factor VIII polypeptide of claim 1, comprising an amino acid sequence having at least 70%, 75%, 80%, 81%, 82%, 83%, 84%, 85%, 86%, 87%, 88%, 89%, 90%, 91%, 92%, 93%, 94%, 95%, 96%, 97%, 98%, or 99% or more sequence identity to the reference sequence corresponding to residues 20-1457 of SEQ ID NO: 2 or to the reference sequence corresponding to SEQ ID NO: 2, wherein the amino acid sequence comprises one or more substitutions relative to the reference sequence corresponding to residues 20-1457 of SEQ ID NO: 2, or relative to the reference sequence corresponding to SEQ ID NO: 2.

3. The recombinant factor VIII polypeptide of claim 1, comprising an amino acid sequence having at least 70%, 75%, 80%, 81%, 82%, 83%, 84%, 85%, 86%, 87%, 88%, 89%, 90%, 91%, 92%, 93%, 94%, 95%, 96%, 97%, 98%, or 99% or more sequence identity to the reference sequence corresponding to residues 20-1457 of SEQ ID NO: 252, 504, 706, 840, 1200, 1514, or 2072, or to the reference sequence corresponding to SEQ ID NO: 2, 252, 504, 706, 840, 1200, 1514, or 2072, wherein the amino acid sequence comprises one or more substitutions relative to the reference sequence corresponding to residues 20-1457 of SEQ ID NO: 2, or relative to the reference sequence corresponding to SEQ ID NO: 2.

4. The recombinant factor VIII polypeptide of claim 1, comprising an amino acid sequence having at least 70%, 75%, 80%, 81%, 82%, 83%, 84%, 85%, 86%, 87%, 88%, 89%, 90%, 91%, 92%, 93%, 94%, 95%, 96%, 97%, 98%, or 99% or more sequence identity to a reference sequence corresponding to residues 20-1457 of an even-numbered SEQ ID NO. of SEQ ID NOs. 4-2346 and 2360-2870, or to a reference sequence corresponding to an even-numbered SEQ ID NO. of SEQ ID NOs. 4-2346 and 2350-2870, wherein the amino acid sequence comprises one or more substitutions relative to the reference sequence corresponding to residues 20-1457 of SEQ ID NO: 2 or relative to the reference sequence corresponding to SEQ ID NO: 2.

5. The recombinant factor VIII polypeptide of claim 1, wherein the amino acid sequence of the recombinant factor VIII polypeptide comprises at least a substitution at amino acid position 13, 14, 15, 16, 17, 18, 19, 20, 21, 24, 37, 56, 57, 58, 59, 61, 63, 64, 65, 85, 96, 104, 110, 119, 127, 132, 158, 160, 164, 167, 168, 171, 175, 178, 186, 187, 205, 210, 211, 212, 215, 225, 246, 257, 260, 265, 281, 292, 294, 301, 303, 304, 306, 308, 310, 311, 312, 318, 320, 321, 325, 328, 332, 334, 335, 344, 346, 347, 349, 351, 353, 354, 355, 356, 358, 359, 362, 370, 387, 388, 390, 392, 393, 397, 407, 428, 429, 433, 435, 436, 481, 487, 488, 489, 491, 498, 499, 503, 514, 527, 533, 534, 535, 536, 538, 543, 550, 551, 555, 558, 569, 580, 581, 584, 610, 611, 612, 613, 614, 615, 616, 617, 618, 619, 620, 621, 622, 643, 646, 650, 665, 667, 676, 680, 681, 682, 683, 684, 686, 693, 696, 698, 700, 706, 707, 708, 712, 716, 724, 726, 729, 731, 732, 734, 735, 736, 737, 738, 739, 740, 744, 746, 747, 748, 749, 750, 751, 752, 753, 754, 755, 756, 757, 759, 770, 773, 775, 785, 786, 787, 790, 791, 792, 793, 794, 795, 797, 798, 799, 800, 801, 802, 805, 806, 807, 808, 809, 810, 811, 812, 815, 816, 817, 818, 819, 821, 822, 824, 825, 826, 828, 830, 831, 836, 837, 838, 839, 840, 841, 844, 866, 873, 879, 892, 896, 897, 903, 907, 914, 916, 917, 918, 919, 921, 922, 923, 924, 926, 929, 938, 939, 945, 948, 951, 968, 999, 1037, 1040, 1041, 1042, 1043, 1049, 1050, 1051, 1053, 1054, 1076, 1078, 1080, 1084, 1088, 1089, 1092, 1093, 1094, 1095, 1098, 1099, 1100, 1101, 1104, 1108, 1109, 1113, 1114, 1116, 1127, 1129, 1138, 1145, 1148, 1151, 1156, 1158, 1160, 1186, 1192, 1193, 1194, 1205, 1210, 1218, 1220, 1221, 1222, 1224, 1229, 1245, 1248, 1250, 1259, 1264, 1269, 1270, 1274, 1281, 1283, 1292, 1297, 1339, 1352, 1373, 1377, 1378, 1380, 1389, 1414, 1422, 1427, 1428, 1439, 1440, 1442, or 1452, or combinations thereof, wherein the amino acid positions are relative to the reference sequence corresponding to SEQ ID NO: 2.

6. The recombinant factor VIII polypeptide of claim 1, wherein the amino acid sequence of the recombinant factor VIII polypeptide comprises at least a substitution or amino acid residue 13G/Y, 14G/M/V, 15G/S, 16A/D/G/P/V, 17S/V, 18L/R, 19G, 20L, 21M, 24V, 37H, 56E/P, 57A/L/S/T/V, 58K, 59D/G/L/M/Q/R/S/V/W, 61Q/S, 63C/E/F/G/H/I/K/L/M/N/Q/R/S/T/W/Y, 64D/G, 65A, 85Q, 96I/L/R/V/W, 104S, 110H, 119G/V, 127V, 132P/R, 158G, 160E/T, 164E, 167G/H/R, 168G/R, 171A/D/E/F/H/K/N/M/P/Q/R/S/T/V/W/Y, 175A/G/H, 178C/M/Q/V/W, 186A, 187C/G, 205R, 210P, 211G/K/P/Q/R, 212A/F/G/K/L/M/N/P/R/S/V/Y, 215E/P/S, 225R, 246D, 257I, 260E/K/T, 265R/S/W, 281A/S, 292D, 294G, 301K/M, 303G/T, 304A/E/G, 306A/D/G/P/S/T, 308P, 310G/V, 311E/G, 312L, 318C/G/K/M/Q/R/T/V, 320G/I/L/P/Q/R/S/T/V, 321Y, 325Y, 328G/Q/S, 332A/F/G/H/N/P/Q/R/T/V/W/Y, 334G, 335E/P, 344A/E/L, 346R/V, 347N, 349G, 351S, 353S, 354R, 355C, 356L/N, 358K/S, 359E/G, 362E/G/N/P/T/V/W, 370A, 387A/G/I/L/Q/R/S/T/V/W, 388A/P, 390A/E/G/L/M/P/S/T/V, 392A, 393A/F/G/H/M/Q/R/W/Y, 397G/N, 407V, 428F, 429L/S, 433A/G, 435Q, 436D/G, 481V, 487N, 488G, 489E/F/Q/R, 491E, 498A, 499C/L, 503S, 514L, 527A, 533R/V/Y, 534F/I/L/M, 535C/G/N, 536A/G/K/L/M/P/R/W, 538V, 543G/L/P/W, 550G, 551W, 555E/G/R/T, 558A/E/G/L/P/Q/R/S/T/V/W, 569T, 580S/Y, 581K/V, 584A, 610H/K/L/R/S/T, 611D/E, 612G/S/T, 613S, 614A/F/H/I/N/T, 615D/N, 616D/E/G/Q/W, 617D/E/G/N, 618C/E/G/P/Y, 619E, 620N/P, 621K/M/R/T/W, 622D, 643N, 646I, 650M, 665G, 667I, 676Q, 680L/R, 681A/C/E/F/G/L/N/R/Q/S/T/W, 682R, 683W, 684G/K, 686W, 693T, 696E, 698D/S/W/Y, 700A/E/Q/T/V, 706A/G/M/Y, 707H, 708A/K/T/V, 712G/L/M/Q/S/T/Y, 716K/L/W, 724I, 726A/L, 729T, 731I/L/R, 732C/D/E/G/L/R/W/K/M/Q/S/T/V/Y, 734I/R/S, 735D/S/V, 736F/G, 737F/G/K/L/M/N/S/V, 738A/D/E/F/H/K/N/P/R/S/T, 739G/P/R, 740G, 744A/R, 746P, 747D, 748F/G/K/N/S/V, 749A/C/D/F/G/I/K/L/M/P/Q/R/S/T/V/Y, 750A/D/G/K/M/N/P/Q/R/S/T/V/W, 751A/D/E/Y, 752D/E/F/G/L/M/Q/R/S/V, 753D, 754W, 755G/L/P/Q/S/Y, 756G/P/R/T, 757A/G/K/T, 759S, 770W, 773H, 775E/G/K/N/P/S/V, 785V, 786C/D/L, 787W, 790A/G/I/M/P/W, 791M/T, 792H/W/Y, 793G, 794R, 795C/N/S/T, 797C/G/P/Q/T/W/Y, 798C/R/T, 799A/E/G/M/P/Q/R/S/V, 800C/G/I/L/M/N/P/Q/R/T/V/W/Y, 801A/G/I/R, 802S/V, 805C/G/H/K/L/M/P/Q/R/S/V/W, 806E/G/R/S, 807C/L/P/R, 808K/S, 809A/C/G/I/K/L/N/Q/R/S/T/V/W, 810V, 811F, 812G/I/V, 815I, 816V/W, 817A/D, 818A/E/G/P/S/V/W, 819V, 821Q, 822S/T, 824W, 825L, 826G, 828K, 830M/V, 831W, 836G/R/V/W, 837G, 838G/T, 839A/G/K/P, 840G, 841W, 844H, 866A, 873L/Q/T, 879Q, 892T, 896L/V, 897G/K/R, 903E, 907D, 914V, 916C/E/I/K/L/M/Q/T, 917G, 918M/S, 919G, 921R/W, 922W, 923M, 924I/M/P/Q/W, 926T/V, 929A/Q/V, 938R/V, 939E/S, 945K, 948S, 951V, 968A/P, 999R, 1037H/M/N/P, 1040E, 1041G/H/N, 1042A/E/T, 1043E/R, 1049G/W, 1050E/P/Q/S/T, 1051E/H/K/R/S/W, 1053A/R, 1054C, 1076G/S, 1078S, 1080G/V, 1084L/N, 1088G/S, 1089F/S, 1092G, 1093A/E/G/M/N/P/Q/R/S/T/V/W/Y, 1094S, 1095G, 1098G, 1099G, 1100I/S, 1101G, 1104C/E, 1108M/S/V/W, 1109G, 1113F, 1114A/M, 1116A/G/I/L/R, 1127L/V, 1129P, 1138Q, 1145R, 1148A/C/E/L/M/N/Q/R/S, 1151S, 1156D/G/N/R, 1158N, 1160R, 1186E, 1192D, 1193A/E/G/I/L, 1194P, 1205L, 1210M/TN/VW, 1218A/G/L/M/P, 1220D/E/L/T, 1221G/S/T/V, 1222F, 1224P, 1229D, 1245R, 1248A/C/D/E/F/G/H/I/K/M/P/Q/R/S/T/W/Y, 1250A/P/S/T, 1259T, 1264L/R/W, 1269F, 1270V, 1274N/V, 1281H/K/P/Q/R/S, 1283E/H/L/S/T, 1292Y, 1297A, 1339C, 1352E, 1373S, 1377A/C/D/E/G/H/I/M/P/R/S/V/T/Y, 1378A/C/D/E, 1380A/G/W, 1389W, 1414A/C/D/E/G/H/N/P/T/W, 1422I, 1427Y, 1428G, 1439A/E/H/L/S/T, 1440A/C/E/G/S/T, 1442T, or 1452L, or combinations thereof, wherein the amino acid positions are relative to the reference sequence corresponding to SEQ ID NO: 2.

7-9. (canceled)

10. The recombinant factor VIII polypeptide of claim 1, wherein the amino acid sequence of the recombinant factor VIII polypeptide comprises at least a substitution or substitution set at amino acid position(s) 1148, 85/708/1148, 798, 706, 171, 335, 63, 344, 428, 879, 59, 429, 205, 1264, 797, 1248, 873, 792, 708, 57, 96, 362, 1292, 346, 433, 65, 795, 684/798, 353, 332, 1259, 356, 436, 1245, 1145, 716, 56, 773, 734, 358, 729, 665, 359, 61, 1250, 726, 37/96/171/429/708/799/1148, 171/429/732/1148, 96/171, 171/919/1151, 171/792/797/799/1148, 171/569/1148, 96/171/429/792/797/799, 171/429/708/1422, 171/429, 321/362/732/797/799, 171/1100, 708/1148/1186, 429/724/732/1148, 171/362/795/797/799, 171/429/732, 96/171/643/797/799, 171/792/797/799, 503, 171/732, 1148/1193, 96/171/362/429/732/795/797, 334, 732, 351, 1377, 999, 306, 349, 257, 646, 435, or 58, wherein the positions are relative to the reference sequence corresponding to SEQ ID NO: 2.

11-13. (canceled)

14. The recombinant factor VIII polypeptide of claim 1, comprising an amino acid sequence having at least 70%, 75%, 80%, 81%, 82%, 83%, 84%, 85%, 86%, 87%, 88%, 89%, 90%, 91%, 92%, 93%, 94%, 95%, 96%, 97%, 98%, or 99% or more sequence identity to the reference sequence corresponding to residues 20-1457 of SEQ ID NO: 252, 504, 706, 840, 1200, 1514, or 2072, or to the reference sequence corresponding to SEQ ID NO: 252, 504, 706, 840, 1200, 1514, or 2072, wherein the amino acid sequence comprises one or more substitutions relative to the reference sequence corresponding to residues 20-1457 of SEQ ID NO: 252, 504, 706, 840, 1200, 1514, or 2072, or relative to the reference sequence corresponding to SEQ ID NO: 252, 504, 706, 840, 1200, 1514, or 2072.

15. The recombinant factor VIII polypeptide of claim 14, wherein the amino acid sequence of the recombinant factor VIII polypeptide comprises at least a substitution at amino acid position 13, 14, 15, 16, 17, 18, 19, 20, 21, 24, 37, 56, 57, 58, 59, 61, 63, 64, 65, 85, 96, 104, 110, 119, 127, 132, 158, 160, 164, 167, 168, 171, 175, 178, 186, 187, 205, 210, 211, 212, 215, 225, 246, 257, 260, 265, 281, 292, 294, 301, 303, 304, 306, 308, 310, 311, 312, 318, 320, 321, 325, 328, 332, 334, 335, 344, 346, 347, 349, 351, 353, 354, 355, 356, 358, 359, 362, 370, 387, 388, 390, 392, 393, 397, 407, 428, 429, 433, 435, 436, 481, 487, 488, 489, 491, 498, 499, 503, 514, 527, 533, 534, 535, 536, 538, 543, 550, 551, 555, 558, 569, 580, 581, 584, 610, 611, 612, 613, 614, 615, 616, 617, 618, 619, 620, 621, 622, 643, 646, 650, 665, 667, 676, 680, 681, 682, 683, 684, 686, 693, 696, 698, 700, 706, 707, 708, 712, 716, 724, 726, 729, 731, 732, 734, 735, 736, 737, 738, 739, 740, 744, 746, 747, 748, 749, 750, 751, 752, 753, 754, 755, 756, 757, 759, 770, 773, 775, 785, 786, 787, 790, 791, 792, 793, 794, 795, 797, 798, 799, 800, 801, 802, 805, 806, 807, 808, 809, 810, 811, 812, 815, 816, 817, 818, 819, 821, 822, 824, 825, 826, 828, 830, 831, 836, 837, 838, 839, 840, 841, 844, 866, 873, 879, 892, 896, 897, 903, 907, 914, 916, 917, 918, 919, 921, 922, 923, 924, 926, 929, 938, 939, 945, 948, 951, 968, 999, 1037, 1040, 1041, 1042, 1043, 1049, 1050, 1051, 1053, 1054, 1076, 1078, 1080, 1084, 1088, 1089, 1092, 1093, 1094, 1095, 1098, 1099, 1100, 1101, 1104, 1108, 1109, 1113, 1114, 1116, 1127, 1129, 1138, 1145, 1148, 1151, 1156, 1158, 1160, 1186, 1192, 1193, 1194, 1205, 1210, 1218, 1220, 1221, 1222, 1224, 1229, 1245, 1248, 1250, 1259, 1264, 1269, 1270, 1274, 1281, 1283, 1292, 1297, 1339, 1352, 1373, 1377, 1378, 1380, 1389, 1414, 1422, 1427, 1428, 1439, 1440, 1442, or 1452, or combinations thereof, wherein the amino acid positions are relative to the reference sequence corresponding to SEQ ID NO: 252, 504, 706, 840, 1200, 1514, or 2072.

16. The recombinant factor VIII polypeptide of claim 14, wherein the amino acid sequence of the recombinant factor VIII polypeptide comprises at least a substitution or amino acid residue 13G/Y, 14G/M/V, 15G/S, 16A/D/G/P/V, 17S/V, 18L/R, 19G, 20L, 21M, 24V, 37H, 56E/P, 57A/L/S/T/V, 58K, 59D/G/L/M/Q/R/S/V/W, 61Q/S, 63C/E/F/G/H/I/K/L/M/N/Q/R/S/T/W/Y, 64D/G, 65A, 85Q, 96I/L/R/V/W, 104S, 110H, 119G/V, 127V, 132P/R, 158G, 160E/T, 164E, 167G/H/R, 168G/R, 171A/D/E/F/H/K/N/M/P/Q/R/S/T/V/W/Y, 175A/G/H, 178C/M/Q/V/W, 186A, 187C/G, 205R, 210P, 211G/K/P/Q/R, 212A/F/G/L/K/M/N/P/R/S/V/Y, 215E/P/S, 225R, 246D, 257I, 260E/K/T, 265R/S/W, 281A/S, 292D, 294G, 301K/M, 303G/T, 304A/E/G, 306A/D/G/P/S/T, 308P, 310G/V, 311E/G, 312L, 318C/G/K/L/M/Q/R/T/V, 320G/I/L/P/Q/R/S/T/V, 321Y, 325Y, 328G/Q/S, 332A/F/G/H/N/P/Q/R/T/V/W/Y, 334G, 335E/P, 344A/E/L, 346R/V, 347N, 349G, 351S, 353S, 354R, 355C, 356L/N, 358K/S, 359E/G, 362E/G/N/P/T/V/W, 370A, 387A/G/I/L/Q/R/S/T/V/W, 388A/P, 390A/E/G/L/M/P/S/T/V, 392A, 393A/F/G/H/M/Q/R/W/Y, 397G/N, 407V, 428F, 429L/S, 433A/G, 435Q, 436D/G, 481V, 487N, 488G, 489E/F/Q/R, 491E, 498A, 499C/L, 503S, 514L, 527A, 533R/V/Y, 534I/F/L/M, 535C/G/N, 536A/G/K/L/M/P/R/W, 538V, 543G/L/P/W, 550G, 551W, 555E/G/R/T, 558A/E/G/L/P/Q/R/S/T/V/W, 569T, 580S1Y, 581K/V, 584A, 610H/K/L/R/S/T, 611D/E, 612G/S/T, 613S, 614A/F/H/I/N/T, 615D/N, 616D/E/G/Q/W, 617D/E/G/N, 618C/E/G/P/Y, 619E, 620N/P, 621K/M/R/T/W, 622D, 643N, 646I, 650M, 665G, 667I, 676Q, 680L/R, 681A/C/E/F/G/L/N/R/Q/S/T/W, 682R, 683W, 684G/K, 686W, 693T, 696E, 698D/S/W/Y, 700A/E/Q/T/V, 706A/G/M/Y, 707H, 708A/K/T/V, 712G/L/M/Q/S/T/Y, 716K/L/W, 724I, 726A/L, 729T, 731I/L/R, 732C/D/E/G/L/R/W/K/M/Q/S/T/V/Y, 734I/R/S, 735D/S/V, 736F/G, 737F/G/K/L/M/N/S/V, 738A/D/E/F/H/K/N/P/R/S/T/Y, 739G/P/R, 740G, 744A/R, 746P, 747D, 748F/G/K/N/S/V, 749A/C/D/F/G/I/K/L/M/P/Q/R/S/T/V/Y, 750A/D/G/K/M/N/P/Q/R/S/T/V/W, 751A/D/E/Y, 752D/E/F/G/L/M/Q/R/S/V, 753D, 754W, 755G/L/P/Q/S/Y, 756G/P/R/T, 757A/G/K/T, 759S, 770W, 773H, 775E/G/K/N/P/S/V, 785V, 786C/D/L, 787W, 790A/G/I/M/P/W, 791M/T, 792H/W/Y, 793G, 794R, 795C/N/S/T, 797C/G/P/Q/T/W/Y, 798C/R/T, 799A/E/G/M/P/Q/R/S/V, 800C/G/I/L/M/N/P/Q/R/T/V/W/Y, 801A/G/I/R, 802S/V, 805C/G/H/K/L/M/P/Q/R/S/V/W, 806E/G/R/S, 807C/L/P/R, 808K/S, 809A/C/G/I/K/L/N/Q/R/S/T/V/W, 810V, 811F, 812G/I/V, 815I, 816V/W, 817A/D, 818A/E/G/P/S/V/W, 819V, 821Q, 822S/T, 824W, 825L, 826G, 828K, 830M/V, 831W, 836G/R/V/W, 837G, 838G/T, 839A/G/K/P, 840G, 841W, 844H, 866A, 873L/Q/T, 879Q, 892T, 896L/V, 897G/K/R, 903E, 907D, 914V, 916C/E/I/K/L/M/Q/T, 917G, 918M/S, 919G, 921R/W, 922W, 923M, 924I/M/P/Q/W, 926T/V, 929A/Q/V, 938R/V, 939E/S, 945K, 948S, 951V, 968A/P, 999R, 1037H/M/N/P, 1040E, 1041G/H/N, 1042A/E/T, 1043E/R, 1049G/W, 1050E/P/Q/S/T, 1051E/H/K/R/S/W, 1053A/R, 1054C, 1076G/S, 1078S, 1080G/V, 1084L/N, 1088G/S, 1089F/S, 1092G, 1093A/E/G/M/N/P/Q/R/S/T/V/W/Y, 1094S, 1095G, 1098G, 1099G, 1100I/S, 1101G, 1104C/E, 1108M/S/V/W, 1109G, 1113F, 1114A/M, 1116A/G/I/L/R, 1127L/V, 1129P, 1138Q, 1145R, 1148A/C/E/L/M/N/Q/R/S, 1151S, 1156D/G/N/R, 1158N, 1160R, 1186E, 1192D, 1193A/E/G/I/L, 1194P, 1205L, 1210M/T/V/W, 1218A/G/L/M/P, 1220D/E/L/T, 1221G/S/T/V, 1222F, 1224P, 1229D, 1245R, 1248A/C/D/E/F/G/H/I/K/M/P/Q/R/S/T/W/Y, 1250A/P/S/T, 1259T, 1264L/R/W, 1269F, 1270V, 1274N/V, 1281H/K/P/Q/R/S, 1283E/H/L/S/T, 1292Y, 1297A, 1339C, 1352E, 1373S, 1377A/C/D/E/G/H/I/M/P/R/S/V/T/Y, 1378A/C/D/E, 1380A/G/W, 1389W, 1414A/C/D/E/G/H/N/P/T/W, 1422I, 1427Y, 1428G, 1439A/E/H/L/S/T, 1440A/C/E/G/S/T, or 1442T, 1452L, or combinations thereof, wherein the amino acid positions are relative to the reference sequence corresponding to SEQ ID NO: 252, 504, 706, 840, 1200, 1514, or 2072.

17-19. (canceled)

20. The recombinant factor VIII polypeptide of claim 14, comprising an amino acid sequence having at least 70%, 75%, 80%, 81%, 82%, 83%, 84%, 85%, 86%, 87%, 88%, 89%, 90%, 91%, 92%, 93%, 94%, 95%, 96%, 97%, 98%, or 99% or more sequence identity to the reference sequence corresponding to residues 20-1457 of SEQ ID NO: 252, or to the reference sequence corresponding to SEQ ID NO: 252, wherein the amino acid sequence comprises one or more substitutions relative to the reference sequence corresponding to residues 20-1457 of SEQ ID NO: 252, or relative to the reference sequence corresponding to SEQ ID NO: 252.

21. The recombinant factor VIII polypeptide of claim 20, wherein the amino acid sequence of the recombinant factor VIII polypeptide comprises at least a substitution or substitution set at amino acid position(s) 1380, 388, 1043, 610, 1274, 1220, 1442, 1193, 621, 1054, 755, 1041, 748, 1049, 1221, 1051, 612, 1037, 616, 387, 489, 750, 1224, 618, 1050, 1053, 896, 897, 749, 260, 481, 613, 756, 63, 265, 752, 1218, 1378, 614, 1440, 1439, 1205, 1222, 1389, 1269, 390, 397/1218, 757, 1373, 346, 63/1248, 349, 306/355/1422, 1422, 306/346/708, 1148, 171/346/1248, 63/797/799/1248/1422, 171/799, 362, 1248, 171/306/346/999/1248, 171/1148, 734/799/1248/1422, 63/732, 63/171/306/346/732, 171, 171/1422, 63/362/732/799/1248, 999/1148/1422, 346/349, 306/349, 63/171/1148/1151, 306, 344/349/354/1250, 1250, 63/306/344/349/354/503/1248, 59/63/332/349/503/706/708, 63/306/344/349/354/706/1248, 59/63, 332/706/708/1248/1250, 503, 59/63/349/354, 59/349/708, 59/306/344/1248, 344/1248/1250, 708, 63/344/349, 59, 63/344/349/354, 706, 59/306/770, 63/347/354/1248, or 354/503, wherein the amino acid positions are relative to the reference sequence corresponding to SEQ ID NO: 252.

22. (canceled)

23. The recombinant factor VIII polypeptide of claim 14, comprising an amino acid sequence having at least 70%, 75%, 80%, 81%, 82%, 83%, 84%, 85%, 86%, 87%, 88%, 89%, 90%, 91%, 92%, 93%, 94%, 95%, 96%, 97%, 98%, or 99% or more sequence identity to the reference sequence corresponding to residues 20-1457 of SEQ ID NO: 504, or to the reference sequence corresponding to SEQ ID NO: 504, wherein the amino acid sequence comprises one or more substitutions relative to the reference sequence corresponding to residues 20-1457 of SEQ ID NO: 504, or relative to the reference sequence corresponding to SEQ ID NO: 504.

24. The recombinant factor VIII polypeptide of claim 23, wherein the amino acid sequence of the recombinant factor VIII polypeptide comprises at least a substitution or substitution set at amino acid position(s) 750, 1051, 260, 756, 1193, 332, 897, 387, 1037, 306/749, 1270, 489, 487, 488, 903, 171/1248/1440, 260/1148/1378, 171/260/387/750/1248, 171/260, 171/260/387/750/1051/1148, 171, 750/1051/1148, 171/260/756, 171/1248, 171/1148, 59, 1440, 332/750/1248, 756/1248, 1248, 171/387/1248/1440, 750/1440, 59/171/750, 171/1193/1248, 750/1148/1193/1440, 1148, 171/387/1051, 748, 306, 306/387/390/749/1220, 390, 306/390/1037, 306/612/749, 387/390/749, 63/390/503/749, 1220, 63/306, 749, 306/390/749/1037, 306/749/1220, 306/387/390/612/749/1037, or 328, wherein the amino acid positions are relative to the reference sequence corresponding to SEQ ID NO: 504.

25. (canceled)

26. The recombinant factor VIII polypeptide of claim 14, comprising an amino acid sequence having at least 70%, 75%, 80%, 81%, 82%, 83%, 84%, 85%, 86%, 87%, 88%, 89%, 90%, 91%, 92%, 93%, 94%, 95%, 96%, 97%, 98%, or 99% or more sequence identity to the reference sequence corresponding to residues 20-1457 of SEQ ID NO: 706, or to the reference sequence corresponding to SEQ ID NO: 706, wherein the amino acid sequence comprises one or more substitutions relative to the reference sequence corresponding to residues 20-1457 of SEQ ID NO: 706, or relative to the reference sequence corresponding to SEQ ID NO: 706.

27. The recombinant factor VIII polypeptide of claim 26, wherein the amino acid sequence of the recombinant factor VIII polypeptide comprises at least a substitution or substitution set at amino acid position(s) 1051, 1221, 1218, 387, 749/1218, 332, 897, 696, 816, 824, 826, 393, 825, 698, 332/387/390/1148, 328/387/749, 749, 306/328/332/390/1051/1193/1218, 63/306/332/1051/1148, 63/306/332/387/1051, 387/390/1051, 186/306/328/332/390/749/1051/1148/1193, 387/390/756/1051, 306/390/1051, 306/332/390/749, 306/387/390/1148, 306/328/332/387/390/749/1051/1148/1193, 332/387/390/749/1148, 387/390/749, 306/387/749/1051/1148, 387/749, 306/945/1051/1193/1221, 306/390/749/1051, 63/306/332/390, 306/387/390/749/1148/1193/1221, 306/332/387/390/749/1218, 63/306/332/390/1051/1148/1193/1218, 924, 755, 752, 751, 923, 797, 754, 793, 753, 922, 896, 938, 775, 732, 810, 747, 393/407, 819, 787, 802, 811, 392, or 397, wherein the amino acid positions are relative to the reference sequence corresponding to SEQ ID NO: 706.

28. (canceled)

29. The recombinant factor VIII polypeptide of claim 14, comprising an amino acid sequence having at least 70%, 75%, 80%, 81%, 82%, 83%, 84%, 85%, 86%, 87%, 88%, 89%, 90%, 91%, 92%, 93%, 94%, 95%, 96%, 97%, 98%, or 99% or more sequence identity to the reference sequence corresponding to residues 20-1457 of SEQ ID NO: 840, or to the reference sequence corresponding to SEQ ID NO: 840, wherein the amino acid sequence comprises one or more substitutions relative to the reference sequence corresponding to residues 20-1457 of SEQ ID NOs: 840, or relative to the reference sequence corresponding to SEQ ID NO: 840.

30. The recombinant factor VIII polypeptide of claim 29, wherein the amino acid sequence of the recombinant factor VIII polypeptide comprises at least a substitution or substitution set at amino acid position(s) 620, 491, 1229, 615, 1283, 822, 328, 212, 818, 555/684, 828, 550, 830, 839, 13, 14, 17, 836, 1210, 1281, 64, 682, 318, 20, 21, 325, 533, 19, 817, 831, 225, 1156, 393/747, 753, 393/732/751, 63/797, 622, 617, 907, 1040, 611, 488, 1192, 1194, 619, 16, 802, 211, 555, 536, 527, 841, 320, 837, 558, 914, 312, 681, 838, 15, 1427, 18, 683, 24, 534, 840, 1160, 551, 393, 393/747/752/811/819/923/1148, 393/751/775, 63/393/732/775/1148, 393/732/1148, 775/1148, 393/752/753/775/793/797, 393/923, 393/923/1148, 393/747/751, 393/747/753/775/1148, 752/923/1051, 63/775, 63/393/752/775/793/1051/1053, 752/753/775/793/819, 63/1051, 63/747/752/753/775/811/819/923/1051, 393/752/787, 751, 63/1148, 63/747/752/753/819/923/1148, 1148, 747/752/753/793/811, 63, 797/1148, 751/752/753/1051, 63/732/1051, 752/797/815/923/1051/1148, or 747/753, wherein the amino acid positions are relative to the reference sequence corresponding to SEQ ID NO: 840.

31. (canceled)

32. The recombinant factor VIII polypeptide of claim 14, comprising an amino acid sequence having at least 70%, 75%, 80%, 81%, 82%, 83%, 84%, 85%, 86%, 87%, 88%, 89%, 90%, 91%, 92%, 93%, 94%, 95%, 96%, 97%, 98%, or 99% or more sequence identity to the reference sequence corresponding to residues 20-1457 of SEQ ID NO: 1200, or to the reference sequence corresponding to SEQ ID NO: 1200, wherein the amino acid sequence comprises one or more substitutions relative to the reference sequence corresponding to residues 20-1457 of SEQ ID NOs: 1200, or relative to the reference sequence corresponding to SEQ ID NO: 1200.

33. The recombinant factor VIII polypeptide of claim 32, wherein the amino acid sequence of the recombinant factor VIII polypeptide comprises at least a substitution or substitution set at amino acid position(s) 681, 622, 617, 558, 536, 211, 320, 839, 797, 533, 751, 303, 951, 164, 818, 807, 799, 735, 794, 926, 755, 921, 801, 1094, 968, 929, 924, 698, 168, 581, 916, 1428, 805, 1114, 311, 737, 693, 809, 757, 1099, 676, 740, 791, 211/536/622/681, 536/558, 211/536/555/617/732, 211/681/732, 211/536/555, 555, 211/536, 211/212, 211/536/617/681/732, 211/536/558/617/681, 555/732, 536/555/558/617/732, 536/617/681/732, 536/555, 536/622/681, 732, 212/320/533/617/681, 211/312/533/534/617/619/681/839, 212, 211/212/320/619/681/839, 797/839, 212/312/320/533/534/617/681, 558/619/681, 320/558/681, 212/320/534/619/681/797/839, 320/558/619/732/839, 211/212/312/534/558/619/681/732/839, 320/534/558/619/732/797, 558/617/619/681, 211/534/558/611/681, 211/320/617/619/681, 558/617/681/797, 320/534/619/732, 211/619/681, 211/212/534/558/619/732/839, 212/320/619/681/797/839, 212/320/611/839, 320/558/619/681, 312/533/617/681/797/839, 558/611/681, 312, 211/320/558/619/681/732/797/839, 320/558/732/797, 533/558/611/619/716/732/839, 312/681, 320/534/619/681, 211/320/533/536/619, 558/617/681/839, 212/320/534/558/681, 533/534/681/839, 212/534/619/681, 617/619/681, 211/212/320/558/617/681, 212/534/558/681/797, 212/320/534, 211/312/619, 212/312/619/839, 210/212/320/558/681, 212/312/534/681/797/839, 211/320/558/839, 294, 759, 538, or 1109, wherein the amino acid positions are relative to the reference sequence corresponding to SEQ ID NO: 1200.

34. (canceled)

35. The recombinant factor VIII polypeptide of claim 14, comprising an amino acid sequence having at least 70%, 75%, 80%, 81%, 82%, 83%, 84%, 85%, 86%, 87%, 88%, 89%, 90%, 91%, 92%, 93%, 94%, 95%, 96%, 97%, 98%, or 99% or more sequence identity to the reference sequence corresponding to residues 20-1457 of SEQ ID NO: 1514, or to the reference sequence corresponding to SEQ ID NO: 1514, wherein the amino acid sequence comprises one or more substitutions relative to the reference sequence corresponding to residues 20-1457 of SEQ ID NOs: 1514, or relative to the reference sequence corresponding to SEQ ID NO: 1514.

36. The recombinant factor VIII polypeptide of claim 35, wherein the amino acid sequence of the recombinant factor VIII polypeptide comprises at least a substitution or substitution set at amino acid position(s) 1042, 1041, 1051, 1283, 1281, 916, 187, 215, 178, 1050, 1210, 104, 615, 610, 536, 618, 621, 614, 110, 301, 1078, 812, 786, 127, 175, 1093, 1088, 650, 535, 806, 738, 700, 132, 1076, 1084, 498, 756, 1108, 746, 160, 543, 310, 584, 918, 1452, 790, 292, 800, 167, 892, 304, 1116, 752, 281, 917, 712, 1080, 806/1352, 1095, 744, 1098, 948, 119, 785, 1113, 667, 158, 580, 707, 731, 1100, 1089, 1092, 939, 1104, 736, 1297, 1127, 686, 1129, 1138, 786/821, 680, 1101, 808, 739, 308, 536/555/619/732/799/805, 555/619/801, 555/619/799, 619/799/801/805, 536/555/619/732/799, 799/805, 801, 246/619/799/801, 536/555/619, 619/839, 619/799/805, 536/801/805, 555/619/799/805, 619/801/805, 555/619, 536/619/801, 801/805, 536/619/799/805, 555/619/732/799/801/805, 749, 616, 611, 370/614, 1156, 1158, 555/681/737/799, 536/619/809/1109, 536/555/619/737/799/809/839/916/921, 916/1109, 536/619/681/799/916/1109, 555/619/681/807/1109, 555/619/681/737/799, 619/681/799/916/1109, 619/681, 555/921, 619/681/916, 555/619/681/799, 555, 732, 619/799, 619, 681, 555/916, 839, or 799, wherein the amino acid positions are relative to the reference sequence corresponding to SEQ ID NO: 1514.

37. (canceled)

38. The recombinant factor VIII polypeptide of claim 14, comprising an amino acid sequence having at least 70%, 75%, 80%, 81%, 82%, 83%, 84%, 85%, 86%, 87%, 88%, 89%, 90%, 91%, 92%, 93%, 94%, 95%, 96%, 97%, 98%, or 99% or more sequence identity to the reference sequence corresponding to residues 20-1457 of SEQ ID NO: 2072, or to the reference sequence corresponding to SEQ ID NO: 2072, wherein the amino acid sequence comprises one or more substitutions relative to the reference sequence corresponding to residues 20-1457 of SEQ ID NOs: 2072, or relative to the reference sequence corresponding to SEQ ID NO: 2072.

39. The recombinant factor VIII polypeptide of claim 38, wherein the amino acid sequence of the recombinant factor VIII comprises at least a substitution or substitution set at amino acid position(s) 167, 167/738/1076/1093, 167/738/752/800/805/808/809/1116, 167/800/809/1076, 167/318, 167/318/800/805/808/809, 167/318/681, 167/318/681/700/805/808/809/1076/1116, 167/318/700, 167, 167/738/1076/1093, 167/738/752/800/805/808/809/1116, 167/800/809/1076, 167/318, 167/318/800/805/808/809, 167/318/681, 167/318/681/700/805/808/809/1076/1116, 167/318/700, 167/318/700/1076, 167/318/700/738/739/1076, 167/681/738/800/808/809, 167/178/700/752/1093, 167/700, 167/700/738/739/1093, 167/700/738/752/1093, 167/700/738/752/805/808/809/1093/1116, 808/809, 1076, 738, 738/808/809, 738/1076, 738/739, 738/739/809, 738/739/809/1093/1116, 738/739/1093, 738/739/752/1093, 738/739/799/800/1093, 738/739/1116, 738/800, 738/1093, 738/752, 738/752/1093, 738/752/799, 738/799/809/1093, 738/799/1093, 739, 739/800/809, 800, 800/805/809, 809, 809/1093, 809/1093/1116, 809/1116, 318, 318/1076, 318/1076/1116, 318/739/805/808/809/1076, 318/499/700/738/809, 318/1093, 318/752/1093, 318/1116, 318/543/739, 318/543/681/752/1116, 318/543/1093, 318/700/738/1093/1414, 681, 681/738, 681/738/739/1093, 681/738/739/752/799/800/809/1116/1339, 681/738/739/1116, 681/738/800, 681/800/805/809, 681/809/1093, 681/752/1093, 681/752/1093, 681/700, 681/700/738/739/1093/1116, 681/700/809, 681/700/1093, 681/700/752/808/809/1093/1116, 681/700/752/1093, 681/700/844, 681/700/805/809, 1093, 1093/1116, 752, 752/1076, 752/800/809, 752/800/1093, 752/809, 752/866, 752/1093, 752/1116, 178/318/739/752/809/1076/1116, 178/318/800/809, 178/318/681/800/808/809/1076, 178/681, 178/681/738/1116, 178/752/809, 178/700/800/805/808/809, 178/805/809/866, 1116, 543, 543/738/739/809, 543/738/809/1093, 543/739/1093, 543/800/1093/1116, 543/809/1093, 543/809/1093/1116, 543/681/738/739/752/809/1093, 543/681/738/739/752/1093, 543/681/739/752/800/805/809/1093, 543/681/700/738/739/800/1093/1116, 543/681/700/738/739/805/808/809, 543/681/700/752, 543/681/700/752/809/1093, 543/1093, 543/700, 543/700/738/739/752/800/1093/1116, 543/700/809, 543/700/1093, 543/700/752, 543/700/752/800/1093, 543/700/752/809/1116, 543/700/752/1093, 700, 700/738/739/800/1093, 700/738/739/799/1093, 700/738/800/1093, 700/738/1116, 700/809, 700/809/1093, 700/1093, 700/1093/1116, 700/752, 700/752/808/809/1093/1116, 700/752/809/1093, 700/752/799/1116, 700/752/805/809/1093, 700/799/800, 700/1116, 700/805/808/809/1076, 514/543/681/805/808/809, 805/808/809, or 805/809/1116, wherein the amino acid positions are relative to the reference sequence corresponding to SEQ ID NO: 2072.

40-42. (canceled)

43. The recombinant factor VIII polypeptide of claim 1, wherein the amino acid sequence of the recombinant factor VIII polypeptide comprises residues 20-1457 of an even-numbered SEQ ID NO. of SEQ ID NOs: 4-2346 and 2360-2870, or comprises an even-numbered SEQ ID NO. of SEQ ID NOs: 4-2346 and 2350-2870, wherein the amino acid sequence optionally includes 1, 2, 3, 4, 5, 6, 7, 8, 9, or up to 10 amino acid substitutions.

44-49. (canceled)

50. The recombinant factor VIII polypeptide of claim 1, wherein the recombinant factor VIII polypeptide exhibits at least one improved property selected from: i) increased activity/potency; ii) increased clotting activity/potency; iii) increased stability to plasma or serum; iv) increased thermostability; v) reduced immunogenicity; vi) increased expression and/or secretion from cells that are transfected with an expression vector; vii) increased transduction and/or secretion from cells that are transduced with a gene therapy vector, or a combination of any of i), ii), iii), iv), v), vi, and/or vii), as compared to a reference factor VIII polypeptide having a sequence corresponding to SEQ ID NO: 2, 252, 504, 706, 840, 1200, 1514, or 2072.

51. (canceled)

52. The recombinant factor VIII polypeptide of claim 1, comprising a deimmunized recombinant factor VIII polypeptide.

53. (canceled)

54. The recombinant factor VIII polypeptide of claim 1, wherein the recombinant factor VIII polypeptide is purified.

55-58. (canceled)

59. A recombinant polynucleotide encoding at least one recombinant factor VIII polypeptide set forth in claim 1.

60-64. (canceled)

65. An expression vector comprising the recombinant polynucleotide of claim 1.

66. (canceled)

67. (canceled)

68. A host cell comprising the expression vector of claim 1.

69. (canceled)

70. (canceled)

71. A method of producing a factor VIII polypeptide, comprising culturing the host cell of claim 68, under conditions such that the factor VIII polypeptide encoded by the recombinant polynucleotide is produced.

72. (canceled)

73. (canceled)

74. A pharmaceutical composition comprising the recombinant factor VIII polypeptide of claim 1.

75. The pharmaceutical composition of claim 74, further comprising a pharmaceutically acceptable carrier and/or excipient.

76. (canceled)

77. A method for treating and/or preventing the symptoms of deficiency in factor VIII in a subject, comprising administering to a subject having a deficiency in factor VIII an effective amount of the recombinant factor VIII polypeptide of claim 1.

78. The method of claim 77, wherein the subject has hemophilia A.

79-82. (canceled)
